(19) United States
(12) Patent Application Publication
(10) Pub. No.: US 2024/0158412 A1
(43) Pub. Date: May 16, 2024

(54) UBIQUITIN-SPECIFIC PROTEASE 7 (USP7) INHIBITORS AND USES THEREOF

(71) Applicant: Faxian Therapeutics, LLC, New York, NY (US)

(72) Inventors: Xianhai HUANG, Warren, NJ (US); Takao SUZUKI, Shanghai (CN); Sarah BOYCE, Brooklyn, NY (US); Yan ZHANG, Roosevelt Island, NY (US); Zhaowu XU, Qidong (CN); Alexandre COTE, West New York, NJ (US); Jeremy GREENWOOD, Brooklyn, NY (US); Heidi KOLDSOE, Brooklyn, NY (US); Eric THERRIEN, Blainville, Quebec (CA); Michael TRZOSS, San Diego, CA (US); Jiayi XU, Marlboro, NJ (US)

(21) Appl. No.: 18/264,352

(22) PCT Filed: Feb. 7, 2022
(86) PCT No.: PCT/US2022/015503

§ 371 Date: Aug. 4, 2023

Related U.S. Application Data
(60) Provisional application No. 63/298,701, filed on Jan. 12, 2022.
(60) Provisional application No. 63/147,015, filed on Feb. 8, 2021.

(51) Int. Cl. C07D 495/04

(52) U.S. Cl. CPC C07D 495/04

(57) ABSTRACT

Described herein are compounds that are useful in treating a USP7-mediated disorder. In some embodiments, the USP7-mediated disorder is cancer.

### CROSS-REFERENCE

This application claims the benefit of U.S. Provisional Application Ser. No. 63/147,015 filed Feb. 8, 2021 and of U.S. Provisional Application Ser. No. 63/298,701 filed Jan. 12, 2022 which are hereby incorporated by reference in their entirety.

### FIELD OF THE INVENTION

Described herein are compounds, methods of making such compounds, pharmaceutical compositions and medicaments comprising such compounds, and methods of using such compounds for inhibiting Ubiquitin-Specific Protease 7 (USP7).

### BACKGROUND OF THE INVENTION

Ubiquitination is a critical post-translational modification of proteins. In this process, chains of ubiquitin (Ub), a small highly conserved regulatory protein, are covalently attached to protein substrates, marking them for degradation in the proteasome. The counteracting de-ubiquitination process removes Ubs from substrate proteins, and thus, rescues the proteins from proteasome-mediated degradation. The ubiquitin-proteasome system (UPS) regulates the degradation of majority proteins in cells. It includes the proteasome, Ubs, the ubiquitin-activating enzymes (E1), the ubiquitin conjugation enzymes (E2), the ubiquitin ligases (E3) and the de-ubiquitinating enzymes (DUB). (Kimura and Tanaka, J. Biochem. 2010, 147(6), 793-798.) Deregulation of UPS has been implicated in the pathogenesis of a wide range of diseases and disorders, including neurodegeneration, infection and immunity, genetic disorders and cancers. (Heideker, J. and Wetz, I. E., Biochem. J. 2015, 465 (1), 1-26; Hussain, S. et. al. Cell Cycle, 2009, 8(11), 1688-1697.)

Ubiquitin-specific proteases (USPs) are the largest family of DUBs. Members of this family are cysteine proteases, which are highly divergent but all contain a conserved catalytic domain. In contrast to other DUB classes, which are thought to generally regulate ubiquitin homeostasis or to be involved in pre-processing of linear ubiquitin chains, USPs remove ubiquitin from specific targets. Given this substrate specificity combined with the numerous roles ubiquitination has in the cell, USPs are important regulators of a multitude of pathways, ranging from preventing the proteolysis of ubiquitinated substrates, to controlling their cellular localization (Fraile, J. M. et. al., Oncogene, 2012, 31, 2373-2388).

USP7 is a USP-family DUB. It was originally identified as an enzyme that interacted with virally-encoded proteins of the herpes simplex virus and later the Epstein-Barr virus (Everett, R. D. et. al. EMBO J. 1997, 16, 1519-1530). In the past decades, USP7 has emerged as a potential therapeutic target due to its regulation of the p53 signaling pathway (Brook, C. L et. al. Oncogene, 2007, 26, 7262-7266; Kon N. et. al. Oncogene, 2010, 29, 1270-1279). p53 is a critical tumor suppressor, which is involved in maintaining cellular homeostasis and frequently mutated in most tumor types. Activation of p53 causes a dramatic reduction in cell proliferation and tumor development (Harris, S. L. and Levine, A. J., Oncogene, 2005, 24, 2899-2908). USP7 deubiquitinates MDM2, an E3 ligase that promotes the ubiquitination of p53. In cancer cells, USP7 is upregulated, and thus, leads to the suppression of p53. USP7 silencing has been shown to increase steady-state p53 levels by promoting MDM2 degradation.

A number of other USP7 targets involved in cancer have recently been identified. Studies suggest that USP7 regulates p16^{INK4a} tumor suppressor through the stabilization of BMI1 and MEL18. USP7 upregulation has been associated with drug resistance to HDAC inhibitors through the stabilization of DNA methyltransferase (DNMT1) (Du, Z. et. al., Sci. Signal., 2010, 3, 1-10.). Genetic mutations of NT5C2 and USP7 are associated with relapsed childhood acute lymphoblastic leukemia (ALL). (Meyer, A. J. et. al., Nat. Genet. 2013, 45, 290-294.) USP7 stabilizes the transcription factor FOXP3, enhancing production of regulatory T-cells (Treg) and suppressing tumor-infiltrating T effector cells. (van Loosdregt, J. et. al. Immunity, 2013, 39, 259-271).

In addition to regulating the protein stability of poly-ubiquitinated targets, USP7 also influences the nuclear accumulation of tumor suppressor PTEN (Song, M. S. et. al. Nature, 455, 813-817). Mono-ubiquitination of PTEN has been shown to affect its cytoplasmic/nuclear partitioning, where nuclear localization of PTEN is important for its tumor suppression activity.

Recent studies have provided an attractive rationale for targeting USP7 in human cancers. Inhibition of USP7 with small molecule inhibitors (Li, P. and Liu, H. M. Eur. J Med. Chem. 2020, 191, 112107), therefore has the potential to be a treatment for cancers and other disorders including neurodegenerative diseases, immunological disorders, cardiovascular diseases, and viral infections.

### BRIEF SUMMARY OF THE INVENTION

Disclosed herein is a compound of Formula (I), or a pharmaceutically acceptable salt, solvate, N-oxide, or stereoisomer thereof:

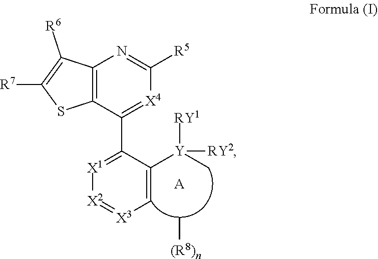

wherein:
Ring A is 5-, 6-, or 7-membered cycloalkyl or heterocycloalkyl;
Y is N; RY¹ is absent; and RY² is -L-R^{A};
or Y is C; RY¹ is hydrogen, deuterium, halogen, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; and RY² is -L-R^{A};
or Y is C and RY¹ and RY² are taken together with Y to form Ring B optionally substituted with one or more R^{B};
L is a bond or C₁-C₆alkylene optionally substituted with one or more deuterium, halogen, —CN, —OR^{b}, —NR^{c}R^{d}, —C(═O)R^{a}, —C(═O)OR^{b}, or —C(═O)NR^{c}R^{d};
R^{A} is —NR^{c}R^{d}, cycloalkyl, heterocycloalkyl, aryl, or heteroaryl; wherein the cycloalkyl, heterocycloalkyl, aryl, and heteroaryl is optionally substituted with one or more R^{Aa};
each R^{Aa} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, alkenyl, alkynyl, cycloalkyl, heterocycloalkyl, aryl, and heteroaryl is optionally substituted with one or more R^{Aaa};
or two R^{Aa} are taken together to form an aryl, heteroaryl, cycloalkyl, or heterocycloalkyl; wherein the aryl, heteroaryl, cycloalkyl, and heterocycloalkyl is optionally substituted with one or more R^{Aaa}; or two R^{Aa} on the same carbon are taken together to form an oxo;
Ring B is cycloalkyl or heterocycloalkyl;
each R^{B} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{B} on the same carbon are taken together to form an oxo;
X¹ is N or CR¹;
R¹ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more Ria;
X² is N or CR²;
R² is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
X³ is N or CR³;
R³ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{3a};
X⁴ is N or CR⁴;
R⁴ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{4a};
R⁵ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{5a};
R⁶ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{6a};
R⁷ is hydrogen, deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, alkenyl, alkynyl, cycloalkyl, heterocycloalkyl, aryl, and heteroaryl is optionally substituted with one or more R^{7a};
each R^{7a} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, alkenyl, alkynyl, cycloalkyl, heterocycloalkyl, aryl, and heteroaryl is optionally substituted with one or more R^{7aa};
or two R^{7a} are taken together to form an aryl, heteroaryl, cycloalkyl, or heterocycloalkyl; wherein the aryl, heteroaryl, cycloalkyl, and heterocycloalkyl is optionally substituted with one or more R^{7aa}. or two R^{7a} on the same carbon are taken together to form an oxo;
each R⁸ is independently hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{5a};
or two R⁸ are taken together to form a cycloalkyl or a heterocycloalkyl; each optionally substituted with one or more R^{5a};
or two R⁸ on the same carbon are taken together to form an oxo;
n is 1-4;
each R^{1a}, R^{2a}, R^{3a}, R^{4a}, R^{5a}, R^{6a}, and R^{8a} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{1a}, or two R^{2a}, or two R^{3a}, or two R^{4a}, or two R^{5a}, or two R^{6a}, or two R^{8a} are taken together to form an a cycloalkyl or a heterocycloalkyl;
or two R^{1a}, or two R^{2a}, or two R^{3a}, or two R^{4a}, or two R^{5a}, or two R^{6a}, or two R^{8a} on the same carbon are taken together to form an oxo;
each R^{7aa} and R^{Aaa} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{7aa} or two R^{Aaa} on the same carbon are taken together to form an oxo;
each R^{a} is independently C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
each R^{b} is independently hydrogen, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; and
each R^{c} and R^{d} is independently hydrogen, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl.

Also disclosed herein is a pharmaceutical composition comprising a compound disclosed herein, or a pharmaceutically acceptable salt, solvate, N-oxide, or stereoisomer thereof, and a pharmaceutically acceptable excipient. In some embodiments, the composition is for use in treating cancer modulated by ubiquitin specific protease 7 (USP7). In some embodiments, the composition is for use in treating solid tumor or blood cancer. In some embodiments, the solid tumor or blood cancer is ovarian cancer, breast cancer, lung cancer, pancreatic cancer, kidney cancer, melanoma, liver cancer, colon cancer, sarcoma, brain cancer, prostate cancer, leukemia, lymphoma, or multiple myeloma.

Also disclosed herein is a method of treating cancer comprising administering to a subject in need thereof a therapeutically effective amount of a compound disclosed herein, or a pharmaceutically acceptable salt, solvate, N-oxide, or stereoisomer thereof. In some embodiments, the cancer is a solid tumor. In some embodiments, the solid tumor is ovarian cancer, breast cancer, lung cancer, pancreatic cancer, kidney cancer, melanoma, liver cancer, colon cancer, sarcoma, brain cancer, or prostate cancer. In some embodiments, the cancer is a blood cancer. In some embodiments, the blood cancer is leukemia, lymphoma, or multiple myeloma. In some embodiments, the leukemia is acute myeloid leukemia (AML). In some embodiments, the blood cancer is a myeloproliferative neoplasm (MPN). In some embodiments, the MPN is chronic myeloid leukemia (CML), chronic neutrophilic leukemia (CNL), polycythemia vera (PV), primary myelofibrosis (PMF), essential thrombocythemia (ET), or chronic eosinophilic leukemia. In some embodiments, the method further comprises administering to the subject in need thereof an additional therapeutic agent. In some embodiments, the additional therapeutic agent is a histone deacetylase (HDAC) inhibitor, a proteasome inhibitor, a BET inhibitor, a B-cell lymphoma 2 (Bcl-2) inhibitor, or any combination thereof. In some embodiments, the additional therapeutic agent is an immunotherapy agent. In some embodiments, the immunotherapy agent is an immune checkpoint inhibitor. In some embodiments, the immune checkpoint inhibitor is a PD-1 inhibitor, a PD-L1 inhibitor, a PD-L2 inhibitor, a CTLA-4 inhibitor, a OX40 agonist, or a 4-1BB agonist.

### INCORPORATION BY REFERENCE

All publications, patents, and patent applications mentioned in this specification are herein incorporated by reference for the specific purposes identified herein.

### DETAILED DESCRIPTION OF THE INVENTION

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Oxo” refers to ═O.

“Alkyl” refers to an optionally substituted straight-chain, or optionally substituted branched-chain saturated hydrocarbon monoradical having from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to, ethenyl (—CH═CH₂), I-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen.

“Alkoxy” refers to a radical of the formula -Oalkyl where alkyl is as defined above. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₅ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Deuteroalkyl” refers to an alkyl radical, as defined above, that is substituted by one or more deuterium atoms. In some embodiments, the alkyl is substituted with one deuterium atom. In some embodiments, the alkyl is substituted with one, two, or three deuterium atoms. In some embodiments, the alkyl is substituted with one, two, three, four, five, or six deuterium atoms. Deuteroalkyl includes, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogen atoms. In some embodiments, the alkyl is substituted with one, two, or three halogen atoms. In some embodiments, the alkyl is substituted with one, two, three, four, five, or six halogen halogens. Haloalkyl includes, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl.

“Halo” or “halogen” refers to bromo, chloro, fluoro or iodo. In some embodiments, halogen is fluoro or chloro. In some embodiments, halogen is fluoro.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocycloalkyl” refers to a 3- to 24-membered partially or fully saturated ring comprising 2 to 23 carbon atoms and from one to 8 heteroatoms selected from the group consisting of nitrogen, oxygen, phosphorous and sulfur. In some embodiments, the heterocycloalkyl comprises 1 or 2 heteroatoms selected from nitrogen and oxygen. Unless stated otherwise specifically in the specification, the heterocycloalkyl may be a monocyclic, bicyclic (including spirocyclic), tricyclic or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). In some embodiments, the heterocycloalkyl is a 3- to 6-membered heterocycloalkyl. In some embodiments, the heterocycloalkyl is a 5- to 6-membered heterocycloalkyl. In some embodiments, the heterocycloalkyl is a 3- to 11-membered heterocycloalkyl. In some embodiments, the heterocycloalkyl is a 6- to 11-membered heterocycloalkyl. Examples of such heterocycloalkyl include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to, the monosaccharides, the disaccharides and the oligosaccharides. The term heterocycloalkyl also includes spirocycles. Examples of such heterocycloalkyl include, but are not limited to, 2-azaspiro[3.3]heptane, 1-azaspiro[3.3]heptane, 2-azaspiro[3.4]octane, 1-azaspiro[3.4]octane, 6-azaspiro[3.4]octane, 5-azaspiro[3.4]octane, 1-azaspiro[4.4]nonane, 2-azaspiro[4.4]nonane, 1-azaspiro[3.5]nonane, 2-azaspiro[3.5]nonane, 5-azaspiro[3.5]nonane, 6-azaspiro[3.5]nonane, 7-azaspiro[3.5]nonane, 1-azaspiro[4.5]decane, 2-azaspiro[4.5]decane, 6-azaspiro[4.5]decane, 7-azaspiro[4.5]decane, 8-azaspiro[4.5]decane, 1-azaspiro[5.5]undecane, 2-azaspiro[5.5]undecane, and 3-azaspiro[5.5]undecane. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heteroaryl” refers to a 5- to 14-membered ring system comprising hydrogen atoms, one to thirteen carbon atoms, one to six heteroatoms selected from the group consisting of nitrogen, oxygen, phosphorous and sulfur, and at least one aromatic ring. The heteroaryl may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease. Also, for purposes herein, “treatment,” “prevention,” “amelioration,” or “inhibition” encompass delaying the onset of the disorder, or a symptom or condition thereof.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

A “USP7 inhibitor” refers to a compound described herein that reduces the function or activity of USP7 when compared to a control, such as, for example, the absence of the compound or a compound with known inactivity. As used herein, the terms “USP7 inhibitor” and “USP7 antagonist” and all other related art-accepted terms, many of which are set forth herein, refer to a compound capable of reducing (e.g., reducing relative to the absence of the inhibitor), either directly or indirectly, the activity and/or function USP7 receptor in an in vitro assay, an in vivo model, and/or other means indicative of therapeutic efficacy. The terms also refer to a compound that exhibits at least some therapeutic benefit in a human subject.

The term “USP7-mediated disease or disorder” refers to a disease or disorder that is characterized by involvement of activity and/or function of USP7 through USP7-mediated pathways in a body.

Compounds

Described herein are compounds that are useful in treating a USP7-mediated disorder. In some embodiments, the USP7-mediated disorder is cancer.

Disclosed herein is a compound of Formula (I), or a pharmaceutically acceptable salt, solvate, N-oxide, or stereoisomer thereof:

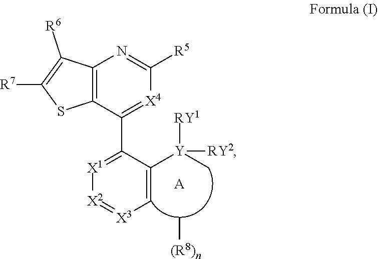

wherein:
Ring A is 5-, 6-, or 7-membered cycloalkyl or heterocycloalkyl;
Y is N; RY¹ is absent; and RY² is -L-R^{A};
or Y is C; RY¹ is hydrogen, deuterium, halogen, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; and RY² is -L-R^{A};
or Y is C and RY¹ and RY² are taken together with Y to form Ring B optionally substituted with one or more R^{B};
L is a bond or C₁-C₆alkylene optionally substituted with one or more deuterium, halogen, —CN, —OR^{b}, —NR^{c}R^{d}, —C(═O)R^{a}, —C(═O)OR^{b}, or —C(═O)NR^{c}R^{d};
R^{A} is —NR^{c}R^{d}, cycloalkyl, heterocycloalkyl, aryl, or heteroaryl; wherein the cycloalkyl, heterocycloalkyl, aryl, and heteroaryl is optionally substituted with one or more R^{Aa};
each R^{Aa} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, alkenyl, alkynyl, cycloalkyl, heterocycloalkyl, aryl, and heteroaryl is optionally substituted with one or more R^{Aaa};
or two R^{Aa} are taken together to form an aryl, heteroaryl, cycloalkyl, or heterocycloalkyl; wherein the aryl, heteroaryl, cycloalkyl, and heterocycloalkyl is optionally substituted with one or more R^{Aaa};
or two R^{Aa} on the same carbon are taken together to form an oxo;
Ring B is cycloalkyl or heterocycloalkyl;
each R^{B} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{B} on the same carbon are taken together to form an oxo;
X¹ is N or CR¹;
R¹ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{1a};
X² is N or CR²;
R² is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
X³ is N or CR³;
R³ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{3a};
X⁴ is N or CR⁴;
R⁴ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{4a};
R⁵ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{5a};
R⁶ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{6a};
R⁷ is hydrogen, deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, alkenyl, alkynyl, cycloalkyl, heterocycloalkyl, aryl, and heteroaryl is optionally substituted with one or more R^{7a};
each R^{7a} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, alkenyl, alkynyl, cycloalkyl, heterocycloalkyl, aryl, and heteroaryl is optionally substituted with one or more R^{7aa};
or two R^{7a} are taken together to form an aryl, heteroaryl, cycloalkyl, or heterocycloalkyl; wherein the aryl, heteroaryl, cycloalkyl, and heterocycloalkyl is optionally substituted with one or more R^{7aa}.
or two R^{7a} on the same carbon are taken together to form an oxo;
each R⁸ is independently hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{5a};
or two R₈ are taken together to form a cycloalkyl or a heterocycloalkyl; each optionally substituted with one or more R^{5a};
or two R⁸ on the same carbon are taken together to form an oxo;
n is 1-4;
each R^{1a}, R^{2a}, R^{3a}, R^{4a}, R^{5a}, R^{6a}, and R^{8a} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{1a}, or two R^{2a}, or two R^{3a}, or two R^{4a}, or two R^{5a}, or two R^{6a}, or two R^{8a} are taken together to form an a cycloalkyl or a heterocycloalkyl;
or two R^{1a}, or two R^{2a}, or two R^{3a}, or two R^{4a}, or two R^{5a}, or two R^{6a}, or two R^{8a} on the same carbon are taken together to form an oxo;
each R^{7aa} and R^{Aaa} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{7aa} or two R^{Aaa} on the same carbon are taken together to form an oxo;
each R^{a} is independently C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
each R^{b} is independently hydrogen, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; and
each R^{c} and R^{d} is independently hydrogen, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl.

In some embodiments of a compound of Formula (I), Y is N; RY¹ is absent; and RY² is -L-R^{A}.

In some embodiments of a compound of Formula (I), Y is C; RY¹ is hydrogen, deuterium, halogen, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; and RY² is -L-R^{A}. In some embodiments of a compound of Formula (I), Y is C; RY¹ is hydrogen or C₁-C₆alkyl; and RY² is -L-R^{A}. In some embodiments of a compound of Formula (I), Y is C; RY¹ is hydrogen; and RY² is -L-R^{A}.

In some embodiments of a compound of Formula (I), L is a bond. In some embodiments of a compound of Formula (I), L is C₁-C₆alkylene optionally substituted with one or more deuterium, halogen, —CN, —OR^{b}, —NR^{c}R^{d}, —C(═O)R^{a}, —C(═O)OR, or —C(═O)NR^{c}R^{d}. In some embodiments of a compound of Formula (I), L is CH₂.

In some embodiments of a compound of Formula (I), L is a bond and R^{A} is cycloalkyl, heterocycloalkyl, aryl, or heteroaryl; wherein the cycloalkyl, heterocycloalkyl, aryl, and heteroaryl is optionally substituted with one or more R^{Aa}.

In some embodiments of a compound of Formula (I), L is CH₂ and R^{A} is —NR^{c}R^{d}, cycloalkyl, heterocycloalkyl, aryl, or heteroaryl; wherein the cycloalkyl, heterocycloalkyl, aryl, and heteroaryl is optionally substituted with one or more R^{Aa}.

In some embodiments of a compound of Formula (I), Y is C and RY¹ and RY² are taken together with Y to form Ring B optionally substituted with one or more R^{B}. In some embodiments ofa compound of Formula (I), Ring B is cycloalkyl. In some embodiments of a compound of Formula (I), Ring B is heterocycloalkyl.

In some embodiments of a compound of Formula (I), Ring B is substituted with one, two, three, or four R^{B}. In some embodiments of a compound of Formula (I), Ring B is substituted with one, two, or three R^{B}. In some embodiments of a compound of Formula (I), Ring B is substituted with one or two R^{B}.

In some embodiments of a compound of Formula (I), each R^{B} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl or two R^{B} on the same carbon are taken together to form an oxo. In some embodiments of a compound of Formula (I), each R^{B} is independently halogen, —CN, —OR^{b}, —NR^{c}R^{d}, C₁-C₆alkyl, C₁-C₆haloalkyl, or C₁-C₆deuteroalkyl or two R^{B} on the same carbon are taken together to form an oxo.

In some embodiments of a compound of Formula (I), each R^{B} is independently deuterium, halogen, —CN, —OR^{b}, —NR^{c}R^{d}, —C(═O)R^{a}, —C(═O)OR, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl. In some embodiments of a compound of Formula (I), each R^{B} is independently halogen, —CN, —OR^{b}, —NR^{c}R^{d}, C₁-C₆alkyl, C₁-C₆haloalkyl, or C₁-C₆deuteroalkyl.

In some embodiments of a compound of Formula (I), Ring A is a 5- or 6-membered cycloalkyl or a 5- or 6-membered heterocycloalkyl.

In some embodiments of a compound of Formula (I), Ring A is a 5- or 6-membered heterocycloalkyl. In some embodiments of a compound of Formula (I), Ring A is 6-membered heterocycloalkyl. In some embodiments of a compound of Formula (I), Ring A is piperidine, piperazine, morpholine, or tetrahydropyran.

In some embodiments of a compound of Formula (I), Ring A is a 5- or 6-membered cycloalkyl. In some embodiments of a compound of Formula (I), Ring A is cyclohexyl.

In some embodiments, the compound of Formula (I), or a pharmaceutically acceptable salt, solvate, N-oxide, or stereoisomer thereof is of Formula (Ia):

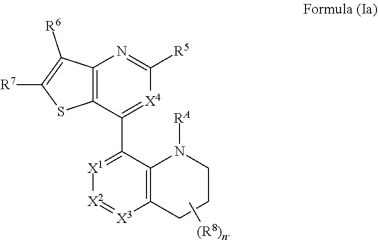

In some embodiments of a compound of Formula (I) or (Ia), X¹ is N. In some embodiments of a compound of Formula (I) or (Ia), X¹ is CR¹.

In some embodiments of a compound of Formula (I) or (Ia), X² is N. In some embodiments of a compound of Formula (I) or (Ia), X² is CR².

In some embodiments of a compound of Formula (I) or (Ia), X³ is N. In some embodiments of a compound of Formula (I) or (Ia), X³ is CR³.

In some embodiments of a compound of Formula (I) or (Ia), X⁴ is N. In some embodiments of a compound of Formula (I) or (Ia), X⁴ is CR⁴.

In some embodiments, the compound of Formula (I) or (Ia), or a pharmaceutically acceptable salt, solvate, N-oxide, or stereoisomer thereof is of Formula (Ib):

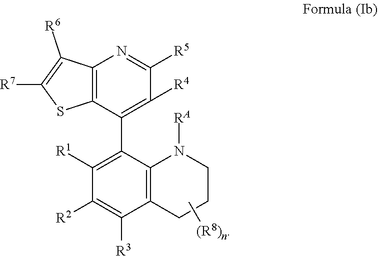

In some embodiments, the compound of Formula (I) or (Ia), or a pharmaceutically acceptable salt, solvate, N-oxide, or stereoisomer thereof is of Formula (Ic):

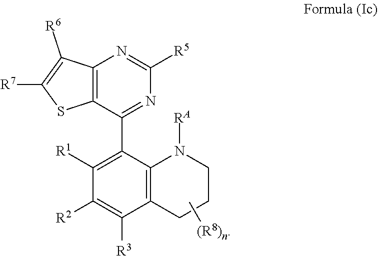

In some embodiments of a compound of Formula (I), (Ia), (Ib), or (Ic), R¹ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{1a}. In some embodiments of a compound of Formula (I), (Ia), (Ib), or (Ic), R¹ is hydrogen, deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; wherein the alkyl, cycloalkyl, and heterocycloalkyl is optionally substituted with one or more R^{1a}. In some embodiments of a compound of Formula (I), (Ia), (Ib), or (Ic), R¹ is hydrogen, deuterium, halogen, —CN, C₁-C₆alkyl, or C₁-C₆haloalkyl; wherein the alkyl is optionally substituted with one or more R^{1a}. In some embodiments of a compound of Formula (I), (Ia), (Ib), or (Ic), R¹ is hydrogen, halogen, —CN, C₁-C₆alkyl, or C₁-C₆haloalkyl. In some embodiments of a compound of Formula (I), (Ia), (Ib), or (Ic), R¹ is hydrogen or halogen. In some embodiments of a compound of Formula (I), (Ia), (Ib), or (Ic), R¹ is halogen. In some embodiments of a compound of Formula (I), (Ia), (Ib), or (Ic), R¹ is hydrogen.

In some embodiments of a compound of Formula (I), (Ia), (Ib), or (Ic), the alkyl, alkenyl, alkynyl, cycloalkyl, heterocycloalkyl, aryl, and heteroaryl of R¹ is optionally substituted with one, two, or three R^{1a}. In some embodiments of a compound of Formula (I), (Ia), (Ib), or (Ic), the alkyl, alkenyl, alkynyl, cycloalkyl, heterocycloalkyl, aryl, and heteroaryl of R¹ is optionally substituted with one or two R^{1a}. In some embodiments of a compound of Formula (I), (Ia), (Ib), or (Ic), the alkyl, alkenyl, alkynyl, cycloalkyl, heterocycloalkyl, aryl, and heteroaryl of R¹ is optionally substituted with one R^{1a}.

In some embodiments of a compound of Formula (I), (Ia), (Ib), or (Ic), each R^{1a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, or heteroaryl; or two R^{1a} are taken together to form an a cycloalkyl or a heterocycloalkyl; or two R^{1a} on the same carbon are taken together to form an oxo. In some embodiments of a compound of Formula (I), (Ia), (Ib), or (Ic), each R^{1a} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{1a} are taken together to form an a cycloalkyl or a heterocycloalkyl; or two R^{1a} on the same carbon are taken together to form an oxo. In some embodiments of a compound of Formula (I), (Ia), (Ib), or (Ic), each R^{1a} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{1a} on the same carbon are taken together to form an oxo. In some embodiments of a compound of Formula (I), (Ia), (Ib), or (Ic), each R^{1a} is independently deuterium, halogen, —CN, —OR^{b}, —NR^{c}R^{d}, C₁-C₆alkyl, or C₁-C₆haloalkyl; or two R^{1a} on the same carbon are taken together to form an oxo. In some embodiments of a compound of Formula (I), (Ia), (Ib), or (Ic), each R^{1a} is independently deuterium, halogen, —CN, —OR^{b}, —NR^{c}R^{d}, C₁-C₆alkyl, or C₁-C₆haloalkyl.

In some embodiments of a compound of Formula (I), (Ia), (Ib), or (Ic), R² is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{2a}. In some embodiments of a compound of Formula (I), (Ia), (Ib), or (Ic), R² is hydrogen, deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; wherein the alkyl, cycloalkyl, and heterocycloalkyl is optionally substituted with one or more R^{2a}. In some embodiments of a compound of Formula (I), (Ia), (Ib), or (Ic), R² is hydrogen, deuterium, halogen, —CN, C₁-C₆alkyl, or C₁-C₆haloalkyl; wherein the alkyl is optionally substituted with one or more R^{2a}. In some embodiments of a compound of Formula (I), (Ia), (Ib), or (Ic), R² is hydrogen, halogen, —CN, C₁-C₆alkyl, or C₁-C₆haloalkyl. In some embodiments of a compound of Formula (I), (Ia), (Ib), or (Ic), R² is hydrogen or halogen. In some embodiments of a compound of Formula (I), (Ia), (Ib), or (Ic), R² is halogen. In some embodiments of a compound of Formula (I), (Ia), (Ib), or (Ic), R² is hydrogen.

In some embodiments of a compound of Formula (I), (Ia), (Ib), or (Ic), the alkyl, alkenyl, alkynyl, cycloalkyl, heterocycloalkyl, aryl, and heteroaryl of R² is optionally substituted with one, two, or three R^{2a}. In some embodiments of a compound of Formula (I), (Ia), (Ib), or (Ic), the alkyl, alkenyl, alkynyl, cycloalkyl, heterocycloalkyl, aryl, and heteroaryl of R² is optionally substituted with one or two R^{2a}. In some embodiments of a compound of Formula (I), (Ia), (Ib), or (Ic), the alkyl, alkenyl, alkynyl, cycloalkyl, heterocycloalkyl, aryl, and heteroaryl of R² is optionally substituted with one R^{2a}.

In some embodiments of a compound of Formula (I), (Ia), (Ib), or (Ic), each R^{2a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, or heteroaryl; or two R^{2a} are taken together to form an a cycloalkyl or a heterocycloalkyl; or two R^{2a} on the same carbon are taken together to form an oxo. In some embodiments of a compound of Formula (I), (Ia), (Ib), or (Ic), each R^{2a} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{2a} are taken together to form an a cycloalkyl or a heterocycloalkyl; or two R^{2a} on the same carbon are taken together to form an oxo. In some embodiments of a compound of Formula (I), (Ia), (Ib), or (Ic), each R^{2a} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{2a} on the same carbon are taken together to form an oxo.

In some embodiments of a compound of Formula (I), (Ia), (Ib), or (Ic), each R^{2a} is independently deuterium, halogen, —CN, —OR^{b}, —NR^{c}R^{d}, C₁-C₆alkyl, or C₁-C₆haloalkyl; or two R^{2a} on the same carbon are taken together to form an oxo. In some embodiments of a compound of Formula (I), (Ia), (Ib), or (Ic), each R^{2a} is independently deuterium, halogen, —CN, —OR^{b}, —NR^{c}R^{d}, C₁-C₆alkyl, or C₁-C₆haloalkyl.

In some embodiments of a compound of Formula (I), (Ia), (Ib), or (Ic), R³ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{3a}. In some embodiments of a compound of Formula (I), (Ia), (Ib), or (Ic), R³ is hydrogen, deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; wherein the alkyl, cycloalkyl, and heterocycloalkyl is optionally substituted with one or more R^{3a}. In some embodiments of a compound of Formula (I), (Ia), (Ib), or (Ic), R³ is hydrogen, deuterium, halogen, —CN, C₁-C₆alkyl, or C₁-C₆haloalkyl; wherein the alkyl is optionally substituted with one or more R^{3a}. In some embodiments of a compound of Formula (I), (Ia), (Ib), or (Ic), R³ is hydrogen, halogen, —CN, C₁-C₆alkyl, or C₁-C₆haloalkyl. In some embodiments of a compound of Formula (I), (Ia), (Ib), or (Ic), R³ is hydrogen or halogen. In some embodiments of a compound of Formula (I), (Ia), (Ib), or (Ic), R³ is halogen. In some embodiments of a compound of Formula (I), (Ia), (Ib), or (Ic), R³ is hydrogen.

In some embodiments of a compound of Formula (I), (Ia), (Ib), or (Ic), the alkyl, alkenyl, alkynyl, cycloalkyl, heterocycloalkyl, aryl, and heteroaryl of R³ is optionally substituted with one, two, or three R^{3a}. In some embodiments of a compound of Formula (I), (Ia), (Ib), or (Ic), the alkyl, alkenyl, alkynyl, cycloalkyl, heterocycloalkyl, aryl, and heteroaryl of R³ is optionally substituted with one or two R^{3a}. In some embodiments of a compound of Formula (I), (Ia), (Ib), or (Ic), the alkyl, alkenyl, alkynyl, cycloalkyl, heterocycloalkyl, aryl, and heteroaryl of R³ is optionally substituted with one R^{3a}.

In some embodiments of a compound of Formula (I), (Ia), (Ib), or (Ic), each R^{3a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, or heteroaryl; or two R^{3a} are taken together to form an a cycloalkyl or a heterocycloalkyl; or two R^{3a} on the same carbon are taken together to form an oxo. In some embodiments of a compound of Formula (I), (Ia), (Ib), or (Ic), each R^{3a} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{3a} are taken together to form an a cycloalkyl or a heterocycloalkyl; or two R^{3a} on the same carbon are taken together to form an oxo. In some embodiments of a compound of Formula (I), (Ia), (Ib), or (Ic), each R^{3a} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{3a} on the same carbon are taken together to form an oxo. In some embodiments of a compound of Formula (I), (Ia), (Ib), or (Ic), each R^{3a} is independently deuterium, halogen, —CN, —OR^{b}, —NR^{c}R^{d}, C₁-C₆alkyl, or C₁-C₆haloalkyl; or two R^{3a} on the same carbon are taken together to form an oxo. In some embodiments of a compound of Formula (I), (Ia), (Ib), or (Ic), each R^{3a} is independently deuterium, halogen, —CN, —OR^{b}, —NR^{c}R^{d}, C₁-C₆alkyl, or C₁-C₆haloalkyl.

In some embodiments of a compound of Formula (I), (Ia), or (Ib), R⁴ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{4a}. In some embodiments of a compound of Formula (I), (Ia), or (Ib), R⁴ is hydrogen, deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; wherein the alkyl, cycloalkyl, and heterocycloalkyl is optionally substituted with one or more R^{4a}. In some embodiments of a compound of Formula (I), (Ia), or (Ib), R⁴ is hydrogen, deuterium, halogen, —CN, C₁-C₆alkyl, or C₁-C₆haloalkyl; wherein the alkyl is optionally substituted with one or more R^{4a}. In some embodiments of a compound of Formula (I), (Ia), or (Ib), R⁴ is hydrogen, halogen, —CN, C₁-C₆alkyl, or C₁-C₆haloalkyl. In some embodiments of a compound of Formula (I), (Ia), or (Ib), R⁴ is hydrogen or halogen. In some embodiments of a compound of Formula (I), (Ia), or (Ib), R⁴ is halogen. In some embodiments of a compound of Formula (I), (Ia), or (Ib), R⁴ is hydrogen.

In some embodiments of a compound of Formula (I), (Ia), or (Ib), the alkyl, alkenyl, alkynyl, cycloalkyl, heterocycloalkyl, aryl, and heteroaryl of R⁴ is optionally substituted with one, two, or three R^{4a}. In some embodiments of a compound of Formula (I), (Ia), or (Ib), the alkyl, alkenyl, alkynyl, cycloalkyl, heterocycloalkyl, aryl, and heteroaryl of R⁴ is optionally substituted with one or two R^{4a}. In some embodiments of a compound of Formula (I), (Ia), or (Ib), the alkyl, alkenyl, alkynyl, cycloalkyl, heterocycloalkyl, aryl, and heteroaryl of R⁴ is optionally substituted with one R^{4a}.

In some embodiments of a compound of Formula (I), (Ia), or (Ib), each R^{4a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, or heteroaryl; or two R^{4a} are taken together to form an a cycloalkyl or a heterocycloalkyl; or two R^{4a} on the same carbon are taken together to form an oxo. In some embodiments of a compound of Formula (I), (Ia), or (Ib), each R^{4a} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{4a} are taken together to form an a cycloalkyl or a heterocycloalkyl; or two R^{4a} on the same carbon are taken together to form an oxo. In some embodiments of a compound of Formula (I), (Ia), or (Ib), each R^{4a} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{4a} on the same carbon are taken together to form an oxo.

In some embodiments of a compound of Formula (I), (Ia), or (Ib), each R^{4a} is independently deuterium, halogen, —CN, —OR^{b}, —NR^{c}R_{d}, C₁-C₆alkyl, or C₁-C₆haloalkyl; or two R^{4a} on the same carbon are taken together to form an oxo. In some embodiments of a compound of Formula (I), (Ia), or (Ib), each R^{4a} is independently deuterium, halogen, —CN, —OR^{b}, —NR^{c}R^{d}, C₁-C₆alkyl, or C₁-C₆haloalkyl.

In some embodiments of a compound of Formula (I), (Ia), (Ib), or (Ic), R is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{5a}. In some embodiments of a compound of Formula (I), (Ia), (Ib), or (Ic), R is hydrogen, deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; wherein the alkyl, cycloalkyl, and heterocycloalkyl is optionally substituted with one or more R^{5a}. In some embodiments of a compound of Formula (I), (Ia), (Ib), or (Ic), R⁵ is hydrogen, deuterium, halogen, —CN, C₁-C₆alkyl, or C₁-C₆haloalkyl; wherein the alkyl is optionally substituted with one or more R^{5a}. In some embodiments of a compound of Formula (I), (Ia), (Ib), or (Ic), R⁵ is hydrogen, halogen, —CN, C₁-C₆alkyl, or C₁-C₆haloalkyl. In some embodiments of a compound of Formula (I), (Ia), (Ib), or (Ic), R is hydrogen or halogen. In some embodiments of a compound of Formula (I), (Ia), (Ib), or (Ic), R⁵ is halogen. In some embodiments of a compound of Formula (I), (Ia), (Ib), or (Ic), R⁵ is hydrogen.

In some embodiments of a compound of Formula (I), (Ia), (Ib), or (Ic), the alkyl, alkenyl, alkynyl, cycloalkyl, heterocycloalkyl, aryl, and heteroaryl of R⁵ is optionally substituted with one, two, or three R^{5a}. In some embodiments of a compound of Formula (I), (Ia), (Ib), or (Ic), the alkyl, alkenyl, alkynyl, cycloalkyl, heterocycloalkyl, aryl, and heteroaryl of R⁵ is optionally substituted with one or two R^{5a}. In some embodiments of a compound of Formula (I), (Ia), (Ib), or (Ic), the alkyl, alkenyl, alkynyl, cycloalkyl, heterocycloalkyl, aryl, and heteroaryl of R is optionally substituted with one R^{5a}.

In some embodiments of a compound of Formula (I), (Ia), (Ib), or (Ic), each R^{5a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, or heteroaryl; or two R^{5a} are taken together to form an a cycloalkyl or a heterocycloalkyl; or two R^{5a} on the same carbon are taken together to form an oxo. In some embodiments of a compound of Formula (I), (Ia), (Ib), or (Ic), each R^{5a} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{5a} are taken together to form an a cycloalkyl or a heterocycloalkyl; or two R^{5a} on the same carbon are taken together to form an oxo. In some embodiments of a compound of Formula (I), (Ia), (Ib), or (Ic), each R^{5a} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{5a} on the same carbon are taken together to form an oxo. In some embodiments of a compound of Formula (I), (Ia), (Ib), or (Ic), each R^{5a} is independently deuterium, halogen, —CN, —OR^{b}, —NR^{c}R^{d}, C₁-C₆alkyl, or C₁-C₆haloalkyl; or two R^{5a} on the same carbon are taken together to form an oxo. In some embodiments of a compound of Formula (I), (Ia), (Ib), or (Ic), each R^{5a} is independently deuterium, halogen, —CN, —OR^{b}, —NR^{c}R^{d}, C₁-C₆alkyl, or C₁-C₆haloalkyl.

In some embodiments of a compound of Formula (I), (Ia), (Ib), or (Ic), R⁶ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{6a}. In some embodiments of a compound of Formula (I), (Ia), (Ib), or (Ic), R⁶ is hydrogen, deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; wherein the alkyl, cycloalkyl, and heterocycloalkyl is optionally substituted with one or more R^{6a}. In some embodiments of a compound of Formula (I), (Ia), (Ib), or (Ic), R⁶ is hydrogen, deuterium, halogen, —CN, C₁-C₆alkyl, or C₁-C₆haloalkyl; wherein the alkyl is optionally substituted with one or more R^{6a}. In some embodiments of a compound of Formula (I), (Ia), (Ib), or (Ic), R⁶ is hydrogen, halogen, —CN, C₁-C₆alkyl, or C₁-C₆haloalkyl. In some embodiments of a compound of Formula (I), (Ia), (Ib), or (Ic), R⁶ is hydrogen or halogen. In some embodiments of a compound of Formula (I), (Ia), (Ib), or (Ic), R⁶ is halogen. In some embodiments of a compound of Formula (I), (Ia), (Ib), or (Ic), R⁶ is hydrogen.

In some embodiments of a compound of Formula (I), (Ia), (Ib), or (Ic), the alkyl, alkenyl, alkynyl, cycloalkyl, heterocycloalkyl, aryl, and heteroaryl of R⁶ is optionally substituted with one, two, or three R^{6a}. In some embodiments of a compound of Formula (I), (Ia), (Ib), or (Ic), the alkyl, alkenyl, alkynyl, cycloalkyl, heterocycloalkyl, aryl, and heteroaryl of R⁶ is optionally substituted with one or two R^{6a}. In some embodiments of a compound of Formula (I), (Ia), (Ib), or (Ic), the alkyl, alkenyl, alkynyl, cycloalkyl, heterocycloalkyl, aryl, and heteroaryl of R⁶ is optionally substituted with one R^{6a}.

In some embodiments of a compound of Formula (I), (Ia), (Ib), or (Ic), each R^{6a} is independently deuterium, halogen, —CN, —OR^{b}, —NRR, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, or heteroaryl; or two R^{6a} are taken together to form an a cycloalkyl or a heterocycloalkyl; or two R^{6a} on the same carbon are taken together to form an oxo. In some embodiments of a compound of Formula (I), (Ia), (Ib), or (Ic), each R^{6a} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{6a} are taken together to form an a cycloalkyl or a heterocycloalkyl; or two R^{6a} on the same carbon are taken together to form an oxo. In some embodiments of a compound of Formula (I), (Ia), (Ib), or (Ic), each R^{6a} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{6a} on the same carbon are taken together to form an oxo. In some embodiments of a compound of Formula (I), (Ia), (Ib), or (Ic), each R^{6a} is independently deuterium, halogen, —CN, —OR^{b}, —NR^{c}R^{d}, C₁-C₆alkyl, or C₁-C₆haloalkyl; or two R^{6a} on the same carbon are taken together to form an oxo. In some embodiments of a compound of Formula (I), (Ia), (Ib), or (Ic), each R^{6a} is independently deuterium, halogen, —CN, —OR^{b}, —NR^{c}R^{d}, C₁-C₆alkyl, or C₁-C₆haloalkyl.

In some embodiments of a compound of Formula (I), (Ia), (Ib), or (Ic), R⁷ is hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, cycloalkyl, heterocycloalkyl, aryl, and heteroaryl is optionally substituted with one or more R^{7a}. In some embodiments of a compound of Formula (I), (Ia), (Ib), or (Ic), R⁷ is —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, cycloalkyl, heterocycloalkyl, aryl, and heteroaryl is optionally substituted with one or more R^{ya}. In some embodiments of a compound of Formula (I), (Ia), (Ib), or (Ic), R⁷ is cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, cycloalkyl, heterocycloalkyl, aryl, and heteroaryl is optionally substituted with one or more R^{7a}. In some embodiments of a compound of Formula (I), (Ia), (Ib), or (Ic), R⁷ is (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, cycloalkyl, heterocycloalkyl, aryl, and heteroaryl is optionally substituted with one or more R^{7a}. In some embodiments of a compound of Formula (I), (Ia), (Ib), or (Ic), R⁷ is (C₁-C₆alkyl)heterocycloalkyl or (C₁-C₆alkyl)heteroaryl; wherein the alkyl, heterocycloalkyl, and heteroaryl is optionally substituted with one or more R^{7a}. In some embodiments of a compound of Formula (I), (Ia), (Ib), or (Ic), R⁷ is (C₁-C₆alkyl)heterocycloalkyl optionally substituted with one or more R^{7a}.

In some embodiments of a compound of Formula (I), (Ia), (Ib), or (Ic), the alkyl, alkenyl, alkynyl, cycloalkyl, heterocycloalkyl, aryl, and heteroaryl of R⁷ is optionally substituted with one, two, three, four, five, or six R^{7a}. In some embodiments of a compound of Formula (I), (Ia), (Ib), or (Ic), the alkyl, alkenyl, alkynyl, cycloalkyl, heterocycloalkyl, aryl, and heteroaryl of R⁷ is optionally substituted with one, two, three, four, or five R^{7a}. In some embodiments of a compound of Formula (I), (Ia), (Ib), or (Ic), the alkyl, alkenyl, alkynyl, cycloalkyl, heterocycloalkyl, aryl, and heteroaryl of R⁷ is optionally substituted with one, two, three, or four R^{7a}. In some embodiments of a compound of Formula (I), (Ia), (Ib), or (Ic), the alkyl, alkenyl, alkynyl, cycloalkyl, heterocycloalkyl, aryl, and heteroaryl of R⁷ is optionally substituted with one, two, or three R^{7a}. In some embodiments of a compound of Formula (I), (Ia), (Ib), or (Ic), the alkyl, alkenyl, alkynyl, cycloalkyl, heterocycloalkyl, aryl, and heteroaryl of R⁷ is optionally substituted with one or two R^{7a}. In some embodiments of a compound of Formula (I), (Ia), (Ib), or (Ic), the alkyl, alkenyl, alkynyl, cycloalkyl, heterocycloalkyl, aryl, and heteroaryl of R⁷ is optionally substituted with one R^{7a}.

In some embodiments of a compound of Formula (I), (Ia), (Ib), or (Ic), each R^{7a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, cycloalkyl, heterocycloalkyl, aryl, and heteroaryl is optionally substituted with one or more R^{7aa}; or two R^{7a} are taken together to form an aryl, heteroaryl, cycloalkyl, or heterocycloalkyl; wherein the aryl, heteroaryl, cycloalkyl, and heterocycloalkyl is optionally substituted with one or more R^{7aa}; or two R^{7a} on the same carbon are taken together to form an oxo. In some embodiments of a compound of Formula (I), (Ia), (Ib), or (Ic), each R^{7a} is independently halogen, —OR^{b}, —NR^{c}R^{d}, C₁-C₆alkyl, C₁-C₆haloalkyl, C₁-C₆heteroalkyl, C₁-C₆hydroxyalkyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, cycloalkyl, heterocycloalkyl, aryl, and heteroaryl is optionally substituted with one or more R^{7aa}; or two R^{7a} are taken together to form an aryl or cycloalkyl; wherein the aryl and cycloalkyl is optionally substituted with one or more R^{7aa}; or two R^{7a} on the same carbon are taken together to form an oxo. In some embodiments of a compound of Formula (I), (Ia), (Ib), or (Ic), each R^{7a} is independently halogen, —OR^{b}, —NR^{c}R^{d}, C₁-C₆alkyl, C₁-C₆haloalkyl, C₁-C₆heteroalkyl, C₁-C₆hydroxyalkyl, cycloalkyl, heterocycloalkyl, heteroaryl, (C₁-C₆alkyl)cycloalkyl, or (C₁-C₆alkyl)heteroaryl; wherein the alkyl, cycloalkyl, heterocycloalkyl, and heteroaryl is optionally substituted with one or more R^{7aa}; or two R^{7a} on the same carbon are taken together to form an oxo. In some embodiments of a compound of Formula (I), (Ia), (Ib), or (Ic), two R^{7a} on the same carbon are taken together to form an oxo.

In some embodiments of a compound of Formula (I), (Ia), (Ib), or (Ic), the alkyl, alkenyl, alkynyl, cycloalkyl, heterocycloalkyl, aryl, and heteroaryl of R^{7a} is optionally substituted with one, two, or three R^{7aa}. In some embodiments of a compound of Formula (I), (Ia), (Ib), or (Ic), the alkyl, alkenyl, alkynyl, cycloalkyl, heterocycloalkyl, aryl, and heteroaryl of R^{7a} is optionally substituted with one or two R^{7aa}. In some embodiments of a compound of Formula (I), (Ia), (Ib), or (Ic), the alkyl, alkenyl, alkynyl, cycloalkyl, heterocycloalkyl, aryl, and heteroaryl of R^{7a} is optionally substituted with one R^{7aa}.

In some embodiments of a compound of Formula (I), (Ia), (Ib), or (Ic), each R^{7aa} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, or heteroaryl; or two R^{7aa} on the same carbon are taken together to form an oxo. In some embodiments of a compound of Formula (I), (Ia), (Ib), or (Ic), each R^{7aa} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{7aa} on the same carbon are taken together to form an oxo. In some embodiments of a compound of Formula (I), (Ia), (Ib), or (Ic), each R^{7aa} is independently deuterium, halogen, —CN, —OR^{b}, —NR^{c}R^{d}, C₁-C₆alkyl, or C₁-C₆haloalkyl; or two R^{7aa} on the same carbon are taken together to form an oxo. In some embodiments of a compound of Formula (I), (Ia), (Ib), or (Ic), each R^{7aa} is independently halogen, —CN, —OR^{b}, —NR^{c}R^{d}, —C(═O)R^{a}, —C(═O)OR^{b}, —C(═O)NR^{c}R^{d}, C₁-C₆alkyl, or C₁-C₆haloalkyl. In some embodiments of a compound of Formula (I), (Ia), (Ib), or (Ic), each R^{7aa} is independently halogen, —CN, —OR^{b}, C₁-C₆alkyl, or C₁-C₆haloalkyl.

In some embodiments of a compound of Formula (I), (Ia), (Ib), or (Ic), R⁷ is

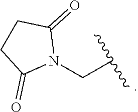 [SMILES: *CN1C(=O)CCC1=O]

In some embodiments of a compound of Formula (I), (Ia), (Ib), or (Ic), n is 1-3. In some embodiments of a compound of Formula (I), (Ia), (Ib), or (Ic), n is 1 or 2. In some embodiments of a compound of Formula (I), (Ia), (Ib), or (Ic), n is 1. In some embodiments of a compound of Formula (I), (Ia), (Ib), or (Ic), n is 2. In some embodiments of a compound of Formula (I), (Ia), (Ib), or (Ic), n is 3. In some embodiments of a compound of Formula (I), (Ia), (Ib), or (Ic), n is 4.

In some embodiments of a compound of Formula (I), (Ia), (Ib), or (Ic), each R⁸ is independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{8a}; or two R⁸ are taken together to form a cycloalkyl or a heterocycloalkyl; each optionally substituted with one or more R^{5a}; or two R⁸ on the same carbon are taken together to form an oxo. In some embodiments of a compound of Formula (I), (Ia), (Ib), or (Ic), each R⁸ is independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; wherein the alkyl is optionally substituted with one or more R^{5a}; or two R⁸ are taken together to form a cycloalkyl or a heterocycloalkyl; each optionally substituted with one or more R^{5a}. In some embodiments of a compound of Formula (I), (Ia), (Ib), or (Ic), each R⁸ is independently hydrogen, deuterium, halogen, —CN, —OR^{b}, —NR^{c}R^{d}, C₁-C₆alkyl, or C₁-C₆haloalkyl; wherein the alkyl is optionally substituted with one or more R^{5a}; or two R⁸ are taken together to form a cycloalkyl or a heterocycloalkyl; each optionally substituted with one or more R^{5a}. In some embodiments of a compound of Formula (I), (Ia), (Ib), or (Ic), each R⁸ is independently hydrogen, deuterium, halogen, C₁-C₆alkyl, or C₁-C₆haloalkyl; or two R⁸ are taken together to form a cycloalkyl or a heterocycloalkyl; or two R⁸ on the same carbon are taken together to form an oxo. In some embodiments of a compound of Formula (I), (Ia), (Ib), or (Ic), each R⁸ is independently hydrogen, deuterium, halogen, C₁-C₆alkyl, or C₁-C₆haloalkyl; or two R⁸ are taken together to form a cycloalkyl or a heterocycloalkyl. In some embodiments of a compound of Formula (I), (Ia), (Ib), or (Ic), two R⁸ are taken together to form a cycloalkyl.

In some embodiments of a compound of Formula (I), (Ia), (Ib), or (Ic), the alkyl, alkenyl, alkynyl, cycloalkyl, heterocycloalkyl, aryl, and heteroaryl of R⁸ is optionally substituted with one, two, or three R^{5a}. In some embodiments of a compound of Formula (I), (Ia), (Ib), or (Ic), the alkyl, alkenyl, alkynyl, cycloalkyl, heterocycloalkyl, aryl, and heteroaryl of R⁸ is optionally substituted with one or two R^{5a}. In some embodiments of a compound of Formula (I), (Ia), (Ib), or (Ic), the alkyl, alkenyl, alkynyl, cycloalkyl, heterocycloalkyl, aryl, and heteroaryl of R⁸ is optionally substituted with one R^{8a}.

In some embodiments of a compound of Formula (I), (Ia), (Ib), or (Ic), each R^{8a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, or heteroaryl; or two R^{8a} are taken together to form an a cycloalkyl or a heterocycloalkyl; or two R^{8a} on the same carbon are taken together to form an oxo. In some embodiments of a compound of Formula (I), (Ia), (Ib), or (Ic), each R^{8a} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{8a} are taken together to form an a cycloalkyl or a heterocycloalkyl; or two R^{8a} on the same carbon are taken together to form an oxo. In some embodiments of a compound of Formula (I), (Ia), (Ib), or (Ic), each R^{8a} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; or two R^{8a} on the same carbon are taken together to form an oxo. In some embodiments of a compound of Formula (I), (Ia), (Ib), or (Ic), each R^{8a} is independently deuterium, halogen, —CN, —OR^{b}, —NR^{c}R^{d}, C₁-C₆alkyl, or C₁-C₆haloalkyl; or two R^{8a} on the same carbon are taken together to form an oxo. In some embodiments of a compound of Formula (I), (Ia), (Ib), or (Ic), each R^{8a} is independently deuterium, halogen, —CN, —OR^{b}, —NR^{c}R^{d}, C₁-C₆alkyl, or C₁-C₆haloalkyl.

In some embodiments of a compound of Formula (I), (Ia), (Ib), or (Ic), R^{A} is NR^{c}R^{d}. In some embodiments of a compound of Formula (I), (Ia), (Ib), or (Ic), R^{A} is cycloalkyl, heterocycloalkyl, aryl, or heteroaryl; wherein the cycloalkyl, heterocycloalkyl, aryl, and heteroaryl is optionally substituted with one or more R^{Aa}. In some embodiments of a compound of Formula (I), (Ia), (Ib), or (Ic), R^{A} is cycloalkyl or heterocycloalkyl; wherein the cycloalkyl and heterocycloalkyl is optionally substituted with one or more R^{A}. In some embodiments of a compound of Formula (I), (Ia), (Ib), or (Ic), R^{A} is cycloalkyl optionally substituted with one or more R^{Aa}. In some embodiments of a compound of Formula (I), (Ia), (Ib), or (Ic), R^{A} is heterocycloalkyl optionally substituted with one or more R^{Aa}. In some embodiments of a compound of Formula (I), (Ia), (Ib), or (Ic), R^{A} is a bicyclic heterocycloalkyl optionally substituted with one or more R^{Aa}.

In some embodiments of a compound of Formula (I), (Ia), (Ib), or (Ic), the alkyl, alkenyl, alkynyl, cycloalkyl, heterocycloalkyl, aryl, and heteroaryl of R^{A} is optionally substituted with one, two, three, four, five, or six R^{Aa}. In some embodiments of a compound of Formula (I), (Ia), (Ib), or (Ic), the alkyl, alkenyl, alkynyl, cycloalkyl, heterocycloalkyl, aryl, and heteroaryl of R^{A} is optionally substituted with one, two, three, four, or five R^{Aa}. In some embodiments of a compound of Formula (I), (Ia), (Ib), or (Ic), the alkyl, alkenyl, alkynyl, cycloalkyl, heterocycloalkyl, aryl, and heteroaryl of R^{A} is optionally substituted with one, two, three, or four R^{Aa}. In some embodiments of a compound of Formula (I), (Ia), (Ib), or (Ic), the alkyl, alkenyl, alkynyl, cycloalkyl, heterocycloalkyl, aryl, and heteroaryl of R^{A} is optionally substituted with one, two, or three R^{Aa}. In some embodiments of a compound of Formula (I), (Ia), (Ib), or (Ic), the alkyl, alkenyl, alkynyl, cycloalkyl, heterocycloalkyl, aryl, and heteroaryl of R^{A} is optionally substituted with one or two R^{Aa}. In some embodiments of a compound of Formula (I), (Ia), (Ib), or (Ic), the alkyl, alkenyl, alkynyl, cycloalkyl, heterocycloalkyl, aryl, and heteroaryl of R^{A} is optionally substituted with one R^{A}.

In some embodiments of a compound of Formula (I), (Ia), (Ib), or (Ic), each R^{Aa} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, alkenyl, alkynyl, cycloalkyl, heterocycloalkyl, aryl, and heteroaryl is optionally substituted with one or more R^{Aaa}; or two R^{Aa} are taken together to form an aryl, heteroaryl, cycloalkyl, or heterocycloalkyl; wherein the aryl, heteroaryl, cycloalkyl, and heterocycloalkyl is optionally substituted with one or more R^{Aaa}; or two R^{Aa} on the same carbon are taken together to form an oxo.

In some embodiments of a compound of Formula (I), (Ia), (Ib), or (Ic), each R^{A} is independently halogen, —CN, —OR^{b}, —NR^{c}R^{d}, —C(═O)R^{a}, —C(═O)OR, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, cycloalkyl, heterocycloalkyl, aryl, and heteroaryl is optionally substituted with one or more R^{Aaa}. In some embodiments of a compound of Formula (I), (Ia), (Ib), or (Ic), each R^{Aa} is independently halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, or heterocycloalkyl; wherein the alkyl, cycloalkyl, and heterocycloalkyl is optionally substituted with one or more R^{Aaa}; or two R^{Aa} are taken together to form an aryl, heteroaryl, cycloalkyl, or heterocycloalkyl; wherein the aryl, heteroaryl, cycloalkyl, and heterocycloalkyl is optionally substituted with one or more R^{Aaa}; or two R^{Aa} on the same carbon are taken together to form an oxo. In some embodiments of a compound of Formula (I), (Ia), (Ib), or (Ic), each R^{Aa} is independently halogen, —CN, —OR^{b}, —NR^{c}R^{d}, —C(═O)R^{a}, —C(═O)OR, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, or heterocycloalkyl; wherein the alkyl, cycloalkyl, and heterocycloalkyl is optionally substituted with one or more R^{Aaa}. In some embodiments of a compound of Formula (I), (Ia), (Ib), or (Ic), each R^{Aa} is independently halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, cycloalkyl, or heterocycloalkyl; wherein the alkyl, cycloalkyl, and heterocycloalkyl is optionally substituted with one or more R^{Aaa}.

In some embodiments of a compound of Formula (I), (Ia), (Ib), or (Ic), the alkyl, alkenyl, alkynyl, cycloalkyl, heterocycloalkyl, aryl, and heteroaryl of R⁸ is optionally substituted with one, two, or three R^{Aaa}. In some embodiments of a compound of Formula (I), (Ia), (Ib), or (Ic), the alkyl, alkenyl, alkynyl, cycloalkyl, heterocycloalkyl, aryl, and heteroaryl of R^{A} is optionally substituted with one or two R^{Aaa}. In some embodiments of a compound of Formula (I), (Ia), (Ib), or (Ic), the alkyl, alkenyl, alkynyl, cycloalkyl, heterocycloalkyl, aryl, and heteroaryl of R^{Aa} is optionally substituted with one R^{Aaa}.

In some embodiments of a compound of Formula (I), (Ia), (Ib), or (Ic), each R^{Aaa} is independently deuterium, halogen, —CN, —OR^{b}, —NR^{c}R^{d}, —C(═O)R^{a}, —C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), (Ib), or (Ic), each R^{Aaa} is independently deuterium, halogen, —CN, —OR^{b}, —NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl. In some embodiments of a compound of Formula (I), (Ia), (Ib), or (Ic), each R^{Aaa} is independently deuterium, halogen, —CN, —OR^{b}, —NR^{c}R^{d}, C₁-C₆alkyl, or C₁-C₆haloalkyl. In some embodiments of a compound of Formula (I), (Ia), (Ib), or (Ic), each R^{Aaa} is independently halogen, —CN, —OR^{b}, C₁-C₆alkyl, or C₁-C₆haloalkyl.

In some embodiments of a compound of Formula (I), (Ia), (Ib), or (Ic), each R^{a} is independently C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), or (Ic), each R^{a} is independently C₁-C₆alkyl, C₁-C₆haloalkyl, cycloalkyl, heterocycloalkyl, aryl, or heteroaryl; wherein each alkyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), or (Ic), each R^{a} is independently C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; wherein each alkyl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), or (Ic), each R^{a} is independently C₁-C₆alkyl or C₁-C₆haloalkyl; wherein each alkyl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), or (Ic), each R^{a} is independently C₁-C₆alkyl, C₁-C₆haloalkyl, C₁-C₆deuteroalkyl, C₁-C₆heteroalkyl, C₁-C₆hydroxyalkyl, or C₁-C₆aminoalkyl. In some embodiments of a compound of Formula (I), (Ia), (Ib), or (Ic), each R^{a} is independently C₁-C₆alkyl or C₁-C₆haloalkyl. In some embodiments of a compound of Formula (I), (Ia), (Ib), or (Ic), each R^{a} is independently C₁-C₆alkyl.

In some embodiments of a compound of Formula (I), (Ia), (Ib), or (Ic), each R^{b} is independently hydrogen, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), or (Ic), each R^{b} is independently hydrogen, C₁-C₆alkyl, C₁-C₆haloalkyl, cycloalkyl, heterocycloalkyl, aryl, or heteroaryl; wherein each alkyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), or (Ic), each R^{b} is independently hydrogen, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; wherein each alkyl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), or (Ic), each R^{b} is independently hydrogen, C₁-C₆alkyl, C₁-C₆haloalkyl, aryl, or heteroaryl; wherein each alkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), or (Ic), each R^{b} is independently hydrogen, C₁-C₆alkyl, or C₁-C₆haloalkyl; wherein each alkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), or (Ic), each R^{b} is independently hydrogen, C₁-C₆alkyl, or C₁-C₆haloalkyl. In some embodiments of a compound of Formula (I), (Ia), (Ib), or (Ic), each R^{b} is independently hydrogen or C₁-C₆alkyl. In some embodiments of a compound of Formula (I), (Ia), (Ib), or (Ic), each R^{b} is independently hydrogen. In some embodiments of a compound of Formula (I), (Ia), (Ib), or (Ic), each R^{b} is independently C₁-C₆alkyl.

In some embodiments of a compound of Formula (I), (Ia), (Ib), or (Ic), each R^{c} and R^{d} is independently hydrogen, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), or (Ic), each R^{c} and R^{d} is independently hydrogen, C₁-C₆alkyl, C₁-C₆haloalkyl, cycloalkyl, heterocycloalkyl, aryl, or heteroaryl; wherein each alkyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), or (Ic), each R^{c} and R^{d} is independently hydrogen, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; wherein each alkyl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), or (Ic), each R^{c} and R^{d} is independently hydrogen, C₁-C₆alkyl, or C₁-C₆haloalkyl; wherein each alkyl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; or R and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), or (Ic), each R^{c} and R^{d} is independently hydrogen, C₁-C₆alkyl, or C₁-C₆haloalkyl; or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), or (Ic), each R and R^{d} is independently hydrogen, C₁-C₆alkyl, or C₁-C₆haloalkyl; or R^{c} and R^{d} are taken together with the nitrogen atom to which they are attached to form a heterocycloalkyl. In some embodiments of a compound of Formula (I), (Ia), (Ib), or (Ic), each R and R^{d} is independently hydrogen, or C₁-C₆alkyl; or R^{c} and R^{d} are taken together with the nitrogen atom to which they are attached to form a heterocycloalkyl. In some embodiments of a compound of Formula (I), (Ia), (Ib), or (Ic), each R^{c} and R^{d} is independently hydrogen; or R^{c} and R^{d} are taken together with the nitrogen atom to which they are attached to form a heterocycloalkyl. In some embodiments of a compound of Formula (I), (Ia), (Ib), or (Ic), each R^{c} and R^{d} is independently C₁-C₆alkyl; or R^{c} and R^{d} are taken together with the nitrogen atom to which they are attached to form a heterocycloalkyl.

In some embodiments of a compound of Formula (I), (Ia), (Ib), or (Ic), the alkyl, alkenyl, alkynyl, cycloalkyl, heterocycloalkyl, aryl, and heteroaryl of R^{a}, R^{b}, R^{c}, and R^{d} is optionally substituted with one, two, or three substituents. In some embodiments of a compound of Formula (I), (Ia), (Ib), or (Ic), the alkyl, alkenyl, alkynyl, cycloalkyl, heterocycloalkyl, aryl, and heteroaryl of R^{a}, R^{b}, R^{c}, and R^{d} is optionally substituted with one or two substituents. In some embodiments of a compound of Formula (I), (Ia), (Ib), or (Ic), the alkyl, alkenyl, alkynyl, cycloalkyl, heterocycloalkyl, aryl, and heteroaryl of R^{a}, R^{b}, R^{c}, and R^{d} is optionally substituted with one substituent.

Disclosed herein is a compound, or a pharmaceutically acceptable salt, solvate, N-oxide, or stereoisomer thereof as found in table 1.

TABLE 1

| Ex. # | Structure |
|---|---|
| 1 | 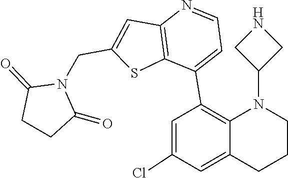 [SMILES: O=C1CCC(=O)N1Cc1cc2nccc(-c3cc(Cl)cc4c3N(C3CNC3)CCC4)c2s1] |
| 2 | 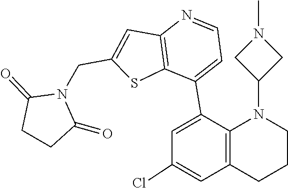 [SMILES: CN1CC(N2CCCc3cc(Cl)cc(-c4ccnc5cc(CN6C(=O)CCC6=O)sc45)c32)C1] |
| 3 | 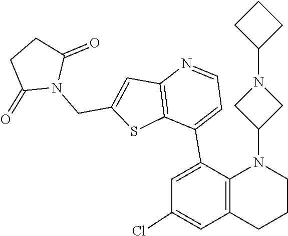 [SMILES: O=C1CCC(=O)N1Cc1cc2nccc(-c3cc(Cl)cc4c3N(C3CN(C5CCC5)C3)CCC4)c2s1] |
| 4 | 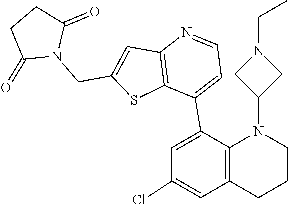 [SMILES: CCN1CC(N2CCCc3cc(Cl)cc(-c4ccnc5cc(CN6C(=O)CCC6=O)sc45)c32)C1] |
| 5 | 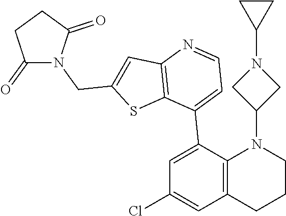 [SMILES: O=C1CCC(=O)N1Cc1cc2nccc(-c3cc(Cl)cc4c3N(C3CN(C5CC5)C3)CCC4)c2s1] |
| 6 | 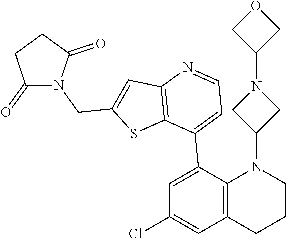 [SMILES: O=C1CCC(=O)N1Cc1cc2nccc(-c3cc(Cl)cc4c3N(C3CN(C5COC5)C3)CCC4)c2s1] |
| 7 | 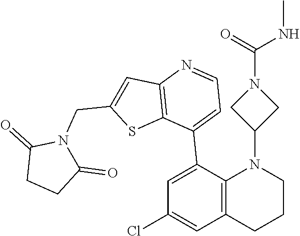 [SMILES: CNC(=O)N1CC(N2CCCc3cc(Cl)cc(-c4ccnc5cc(CN6C(=O)CCC6=O)sc45)c32)C1] |
| 8 | 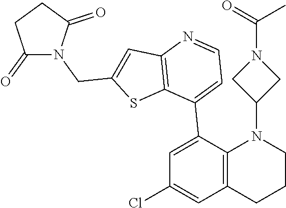 [SMILES: CC(=O)N1CC(N2CCCc3cc(Cl)cc(-c4ccnc5cc(CN6C(=O)CCC6=O)sc45)c32)C1] |
| 9 | 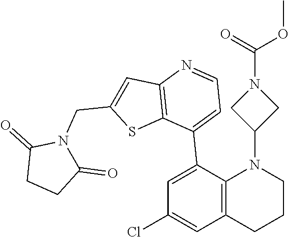 [SMILES: COC(=O)N1CC(N2CCCc3cc(Cl)cc(-c4ccnc5cc(CN6C(=O)CCC6=O)sc45)c32)C1] |
| 10 | 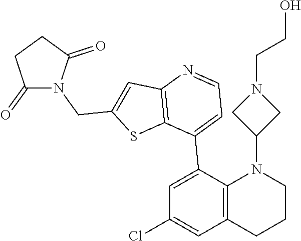 [SMILES: O=C1CCC(=O)N1Cc1cc2nccc(-c3cc(Cl)cc4c3N(C3CN(CCO)C3)CCC4)c2s1] |
| 11 | 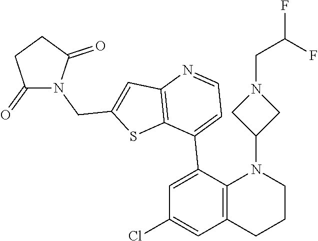 [SMILES: O=C1CCC(=O)N1Cc1cc2nccc(-c3cc(Cl)cc4c3N(C3CN(CC(F)F)C3)CCC4)c2s1] |
| 12 | 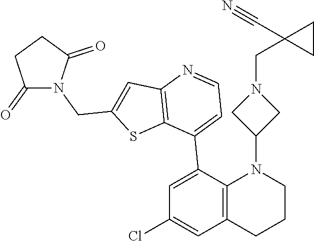 [SMILES: N#CC1(CN2CC(N3CCCc4cc(Cl)cc(-c5ccnc6cc(CN7C(=O)CCC7=O)sc56)c43)C2)CC1] |
| 13 | 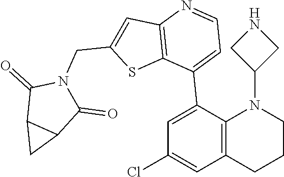 [SMILES: O=C1C2CC2C(=O)N1Cc1cc2nccc(-c3cc(Cl)cc4c3N(C3CNC3)CCC4)c2s1] |
| 14 | 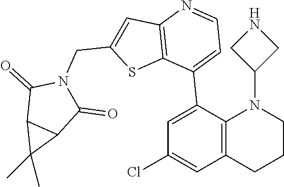 [SMILES: CC1(C)C2C(=O)N(Cc3cc4nccc(-c5cc(Cl)cc6c5N(C5CNC5)CCC6)c4s3)C(=O)C21] |
| 15 | 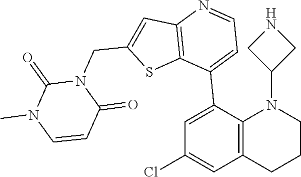 [SMILES: Cn1ccc(=O)n(Cc2cc3nccc(-c4cc(Cl)cc5c4N(C4CNC4)CCC5)c3s2)c1=O] |
| 16 | 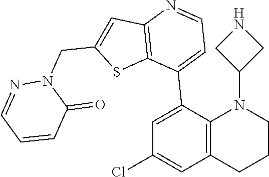 [SMILES: O=c1cccnn1Cc1cc2nccc(-c3cc(Cl)cc4c3N(C3CNC3)CCC4)c2s1] |
| 17 | 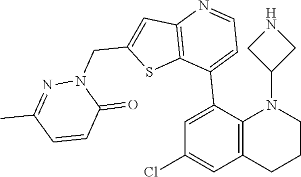 [SMILES: Cc1ccc(=O)n(Cc2cc3nccc(-c4cc(Cl)cc5c4N(C4CNC4)CCC5)c3s2)n1] |
| 18 | 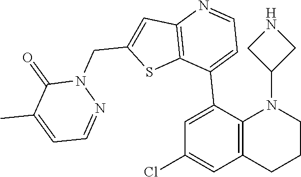 [SMILES: Cc1ccnn(Cc2cc3nccc(-c4cc(Cl)cc5c4N(C4CNC4)CCC5)c3s2)c1=O] |
| 19 | 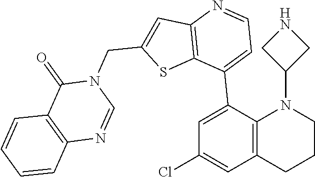 [SMILES: O=c1c2ccccc2ncn1Cc1cc2nccc(-c3cc(Cl)cc4c3N(C3CNC3)CCC4)c2s1] |
| 20 | 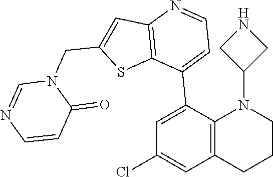 [SMILES: O=c1ccncn1Cc1cc2nccc(-c3cc(Cl)cc4c3N(C3CNC3)CCC4)c2s1] |
| 21 | 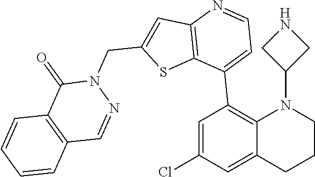 [SMILES: O=c1c2ccccc2cnn1Cc1cc2nccc(-c3cc(Cl)cc4c3N(C3CNC3)CCC4)c2s1] |
| 22 | 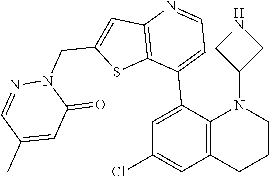 [SMILES: Cc1cnn(Cc2cc3nccc(-c4cc(Cl)cc5c4N(C4CNC4)CCC5)c3s2)c(=O)c1] |
| 23 | 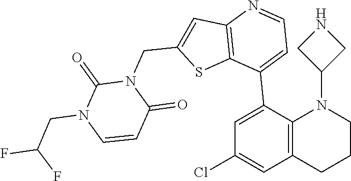 [SMILES: O=c1ccn(CC(F)F)c(=O)n1Cc1cc2nccc(-c3cc(Cl)cc4c3N(C3CNC3)CCC4)c2s1] |
| 24 | 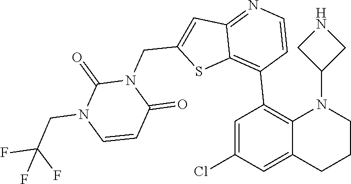 [SMILES: O=c1ccn(CC(F)(F)F)c(=O)n1Cc1cc2nccc(-c3cc(Cl)cc4c3N(C3CNC3)CCC4)c2s1] |
| 25 | 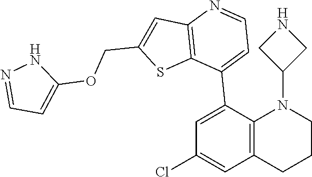 [SMILES: Clc1cc2c(c(-c3ccnc4cc(COc5ccn[nH]5)sc34)c1)N(C1CNC1)CCC2] |
| 26 | 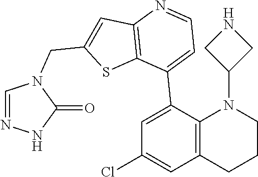 [SMILES: O=c1[nH]ncn1Cc1cc2nccc(-c3cc(Cl)cc4c3N(C3CNC3)CCC4)c2s1] |
| 27 | 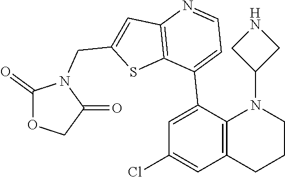 [SMILES: O=C1COC(=O)N1Cc1cc2nccc(-c3cc(Cl)cc4c3N(C3CNC3)CCC4)c2s1] |
| 28 | 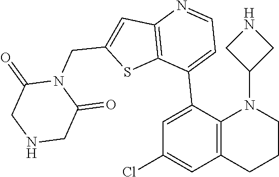 [SMILES: O=C1CNCC(=O)N1Cc1cc2nccc(-c3cc(Cl)cc4c3N(C3CNC3)CCC4)c2s1] |
| 29 | 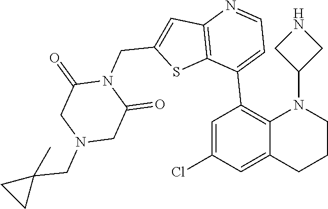 [SMILES: CC1(CN2CC(=O)N(Cc3cc4nccc(-c5cc(Cl)cc6c5N(C5CNC5)CCC6)c4s3)C(=O)C2)CC1] |
| 30 | 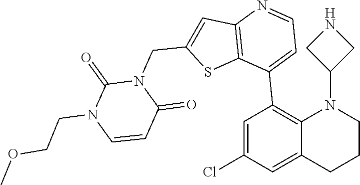 [SMILES: COCCn1ccc(=O)n(Cc2cc3nccc(-c4cc(Cl)cc5c4N(C4CNC4)CCC5)c3s2)c1=O] |
| 31 | 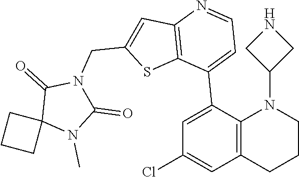 [SMILES: CN1C(=O)N(Cc2cc3nccc(-c4cc(Cl)cc5c4N(C4CNC4)CCC5)c3s2)C(=O)C12CCC2] |
| 32 | 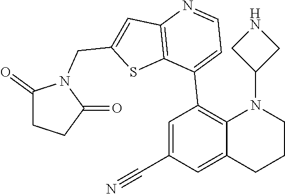 [SMILES: N#Cc1cc2c(c(-c3ccnc4cc(CN5C(=O)CCC5=O)sc34)c1)N(C1CNC1)CCC2] |
| 33 | 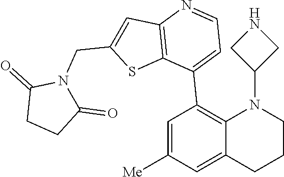 [SMILES: Cc1cc2c(c(-c3ccnc4cc(CN5C(=O)CCC5=O)sc34)c1)N(C1CNC1)CCC2] |
| 34 | 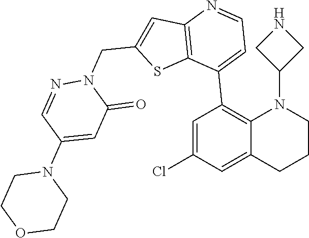 [SMILES: O=c1cc(N2CCOCC2)cnn1Cc1cc2nccc(-c3cc(Cl)cc4c3N(C3CNC3)CCC4)c2s1] |
| 35 | 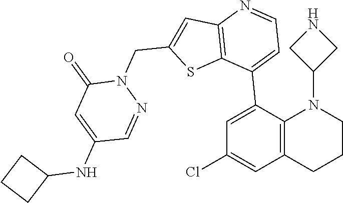 [SMILES: O=c1cc(NC2CCC2)cnn1Cc1cc2nccc(-c3cc(Cl)cc4c3N(C3CNC3)CCC4)c2s1] |
| 36 | 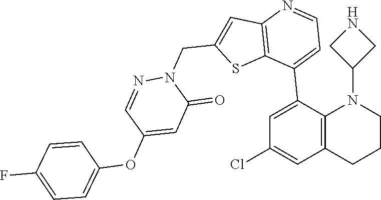 [SMILES: O=c1cc(Oc2ccc(F)cc2)cnn1Cc1cc2nccc(-c3cc(Cl)cc4c3N(C3CNC3)CCC4)c2s1] |
| 37 | 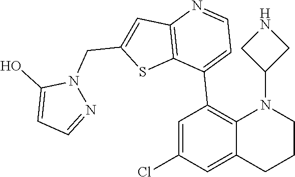 [SMILES: Oc1ccnn1Cc1cc2nccc(-c3cc(Cl)cc4c3N(C3CNC3)CCC4)c2s1] |
| 38 | 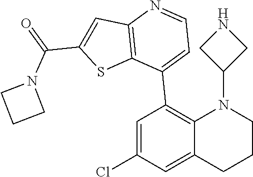 [SMILES: O=C(c1cc2nccc(-c3cc(Cl)cc4c3N(C3CNC3)CCC4)c2s1)N1CCC1] |
| 39 | 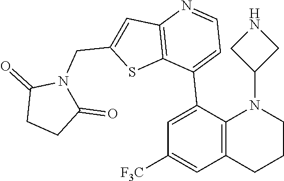 [SMILES: O=C1CCC(=O)N1Cc1cc2nccc(-c3cc(C(F)(F)F)cc4c3N(C3CNC3)CCC4)c2s1] |
| 40 | 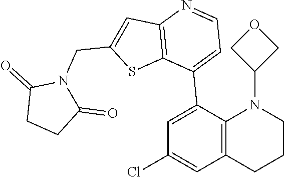 [SMILES: O=C1CCC(=O)N1Cc1cc2nccc(-c3cc(Cl)cc4c3N(C3COC3)CCC4)c2s1] |
| 41 | 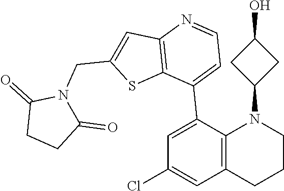 [SMILES: O=C1CCC(=O)N1Cc1cc2nccc(-c3cc(Cl)cc4c3N([C@H]3C[C@@H](O)C3)CCC4)c2s1] |
| 42a 42b | 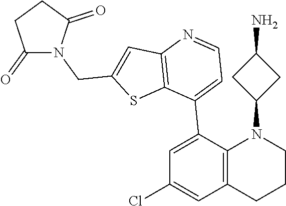 [SMILES: N[C@H]1C[C@@H](N2CCCc3cc(Cl)cc(-c4ccnc5cc(CN6C(=O)CCC6=O)sc45)c32)C1] |
| | 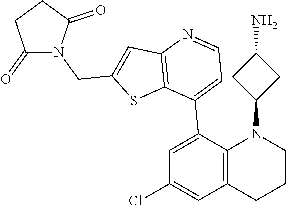 [SMILES: N[C@H]1C[C@H](N2CCCc3cc(Cl)cc(-c4ccnc5cc(CN6C(=O)CCC6=O)sc45)c32)C1] |
| 43a 43b | 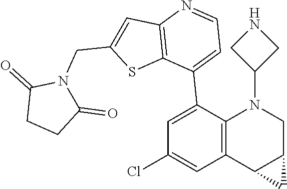 [SMILES: O=C1CCC(=O)N1Cc1cc2nccc(-c3cc(Cl)cc4c3N(C3CNC3)C[C@@H]3C[C@H]43)c2s1] |
| | 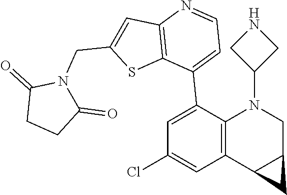 [SMILES: O=C1CCC(=O)N1Cc1cc2nccc(-c3cc(Cl)cc4c3N(C3CNC3)C[C@H]3C[C@@H]43)c2s1] |
| 44a 44b 44c 44d | 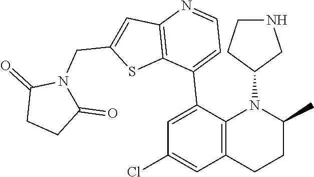 [SMILES: C[C@H]1CCc2cc(Cl)cc(-c3ccnc4cc(CN5C(=O)CCC5=O)sc34)c2N1[C@@H]1CCNC1] |
| | 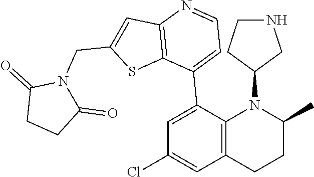 [SMILES: C[C@H]1CCc2cc(Cl)cc(-c3ccnc4cc(CN5C(=O)CCC5=O)sc34)c2N1[C@H]1CCNC1] |
| | 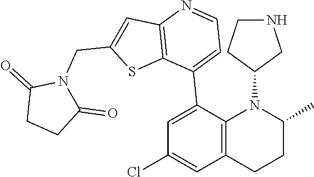 [SMILES: C[C@@H]1CCc2cc(Cl)cc(-c3ccnc4cc(CN5C(=O)CCC5=O)sc34)c2N1[C@@H]1CCNC1] |
| | 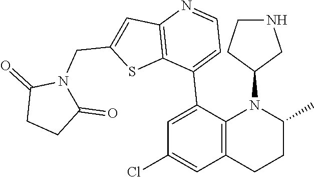 [SMILES: C[C@@H]1CCc2cc(Cl)cc(-c3ccnc4cc(CN5C(=O)CCC5=O)sc34)c2N1[C@H]1CCNC1] |
| 45 | 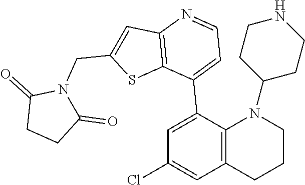 [SMILES: O=C1CCC(=O)N1Cc1cc2nccc(-c3cc(Cl)cc4c3N(C3CCNCC3)CCC4)c2s1] |
| 46 | 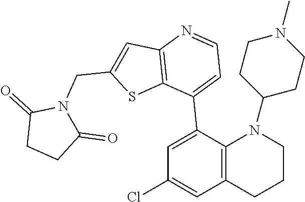 [SMILES: CN1CCC(N2CCCc3cc(Cl)cc(-c4ccnc5cc(CN6C(=O)CCC6=O)sc45)c32)CC1] |
| 47a 47b | 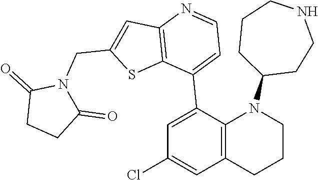 [SMILES: O=C1CCC(=O)N1Cc1cc2nccc(-c3cc(Cl)cc4c3N([C@H]3CCCNCC3)CCC4)c2s1] |
| | 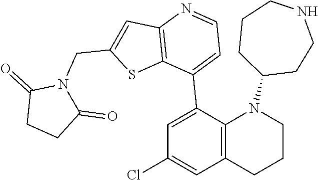 [SMILES: O=C1CCC(=O)N1Cc1cc2nccc(-c3cc(Cl)cc4c3N([C@@H]3CCCNCC3)CCC4)c2s1] |
| 48 | 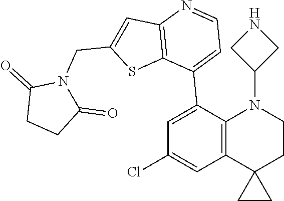 [SMILES: O=C1CCC(=O)N1Cc1cc2nccc(-c3cc(Cl)cc4c3N(C3CNC3)CCC43CC3)c2s1] |
| 49 | 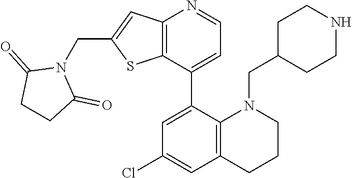 [SMILES: O=C1CCC(=O)N1Cc1cc2nccc(-c3cc(Cl)cc4c3N(CC3CCNCC3)CCC4)c2s1] |
| 50 | 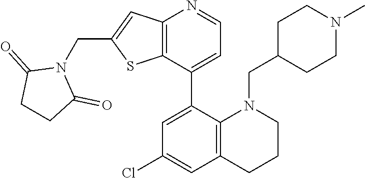 [SMILES: CN1CCC(CN2CCCc3cc(Cl)cc(-c4ccnc5cc(CN6C(=O)CCC6=O)sc45)c32)CC1] |
| 51a 51b | 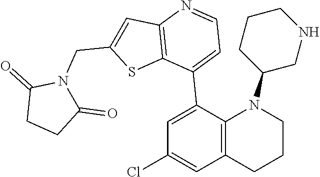 [SMILES: O=C1CCC(=O)N1Cc1cc2nccc(-c3cc(Cl)cc4c3N([C@H]3CCCNC3)CCC4)c2s1] |
| | 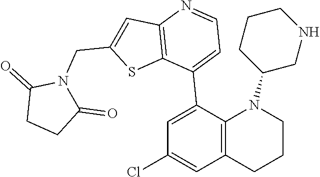 [SMILES: O=C1CCC(=O)N1Cc1cc2nccc(-c3cc(Cl)cc4c3N([C@@H]3CCCNC3)CCC4)c2s1] |
| 52a 52b | 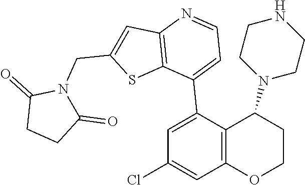 [SMILES: O=C1CCC(=O)N1Cc1cc2nccc(-c3cc(Cl)cc4c3[C@H](N3CCNCC3)CCO4)c2s1] |
| | 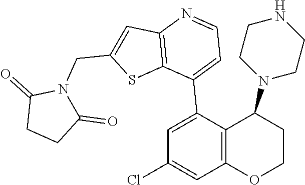 [SMILES: O=C1CCC(=O)N1Cc1cc2nccc(-c3cc(Cl)cc4c3[C@@H](N3CCNCC3)CCO4)c2s1] |
| 53 | 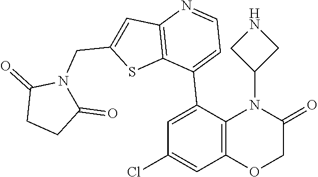 [SMILES: O=C1CCC(=O)N1Cc1cc2nccc(-c3cc(Cl)cc4c3N(C3CNC3)C(=O)CO4)c2s1] |
| 54 | 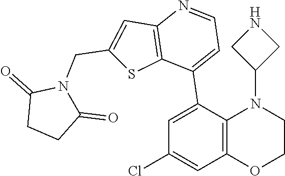 [SMILES: O=C1CCC(=O)N1Cc1cc2nccc(-c3cc(Cl)cc4c3N(C3CNC3)CCO4)c2s1] |
| 55 | 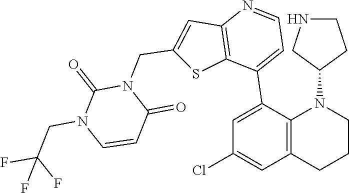 [SMILES: O=c1ccn(CC(F)(F)F)c(=O)n1Cc1cc2nccc(-c3cc(Cl)cc4c3N([C@H]3CCNC3)CCC4)c2s1] |
| 56 | 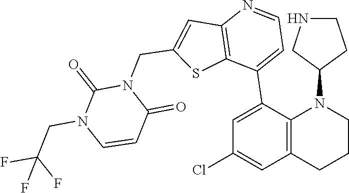 [SMILES: O=c1ccn(CC(F)(F)F)c(=O)n1Cc1cc2nccc(-c3cc(Cl)cc4c3N([C@@H]3CCNC3)CCC4)c2s1] |
| 57 | 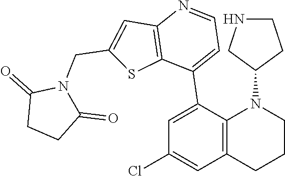 [SMILES: O=C1CCC(=O)N1Cc1cc2nccc(-c3cc(Cl)cc4c3N([C@H]3CCNC3)CCC4)c2s1] |
| 58 | 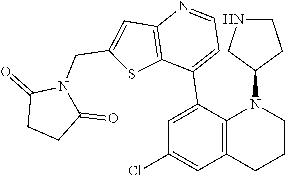 [SMILES: O=C1CCC(=O)N1Cc1cc2nccc(-c3cc(Cl)cc4c3N([C@@H]3CCNC3)CCC4)c2s1] |
| 59 | 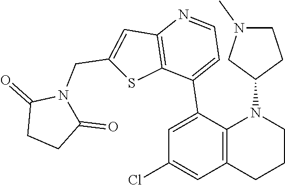 [SMILES: CN1CC[C@H](N2CCCc3cc(Cl)cc(-c4ccnc5cc(CN6C(=O)CCC6=O)sc45)c32)C1] |
| 60 | 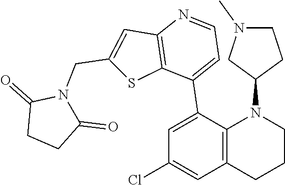 [SMILES: CN1CC[C@@H](N2CCCc3cc(Cl)cc(-c4ccnc5cc(CN6C(=O)CCC6=O)sc45)c32)C1] |
| 61 | 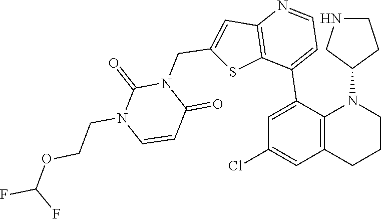 [SMILES: O=c1ccn(CCOC(F)F)c(=O)n1Cc1cc2nccc(-c3cc(Cl)cc4c3N([C@H]3CCNC3)CCC4)c2s1] |
| 62 | 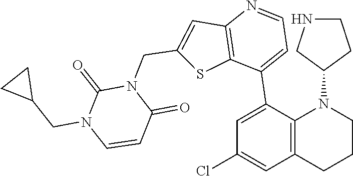 [SMILES: O=c1ccn(CC2CC2)c(=O)n1Cc1cc2nccc(-c3cc(Cl)cc4c3N([C@H]3CCNC3)CCC4)c2s1] |
| 63 | 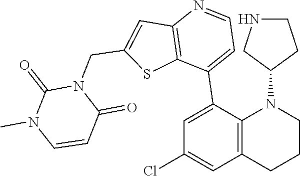 [SMILES: Cn1ccc(=O)n(Cc2cc3nccc(-c4cc(Cl)cc5c4N([C@H]4CCNC4)CCC5)c3s2)c1=O] |
| 64 | 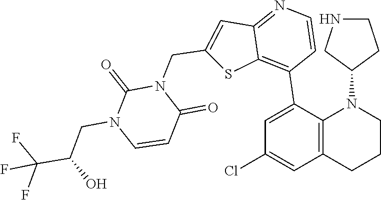 [SMILES: O=c1ccn(C[C@H](O)C(F)(F)F)c(=O)n1Cc1cc2nccc(-c3cc(Cl)cc4c3N([C@H]3CCNC3)CCC4)c2s1] |
| 65 | 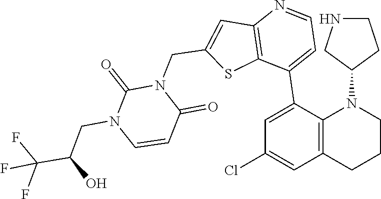 [SMILES: O=c1ccn(C[C@@H](O)C(F)(F)F)c(=O)n1Cc1cc2nccc(-c3cc(Cl)cc4c3N([C@H]3CCNC3)CCC4)c2s1] |
| 66 | 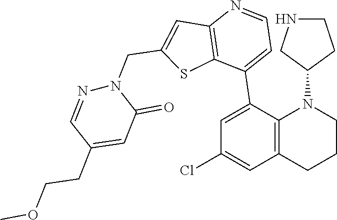 [SMILES: COCCc1cnn(Cc2cc3nccc(-c4cc(Cl)cc5c4N([C@H]4CCNC4)CCC5)c3s2)c(=O)c1] |
| 67 | 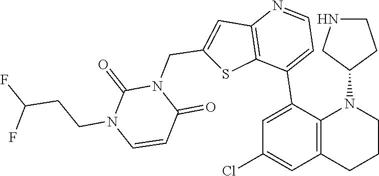 [SMILES: O=c1ccn(CCC(F)F)c(=O)n1Cc1cc2nccc(-c3cc(Cl)cc4c3N([C@H]3CCNC3)CCC4)c2s1] |
| 68 | 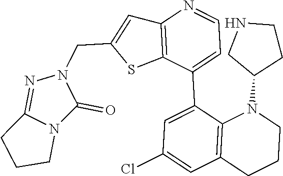 [SMILES: O=c1n(Cc2cc3nccc(-c4cc(Cl)cc5c4N([C@H]4CCNC4)CCC5)c3s2)nc2n1CCC2] |
| 69 | 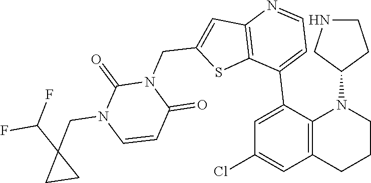 [SMILES: O=c1ccn(CC2(C(F)F)CC2)c(=O)n1Cc1cc2nccc(-c3cc(Cl)cc4c3N([C@H]3CCNC3)CCC4)c2s1] |
| 70 | 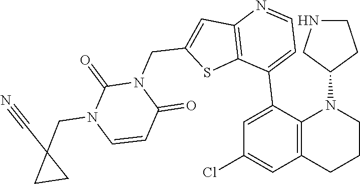 [SMILES: N#CC1(Cn2ccc(=O)n(Cc3cc4nccc(-c5cc(Cl)cc6c5N([C@H]5CCNC5)CCC6)c4s3)c2=O)CC1] |
| 71 | 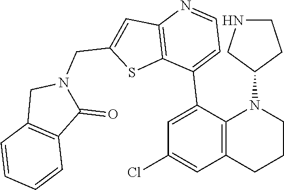 [SMILES: O=C1c2ccccc2CN1Cc1cc2nccc(-c3cc(Cl)cc4c3N([C@H]3CCNC3)CCC4)c2s1] |
| 72 | 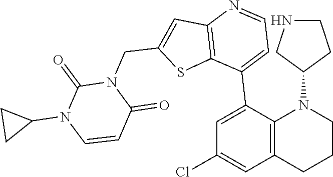 [SMILES: O=c1ccn(C2CC2)c(=O)n1Cc1cc2nccc(-c3cc(Cl)cc4c3N([C@H]3CCNC3)CCC4)c2s1] |
| 73 | 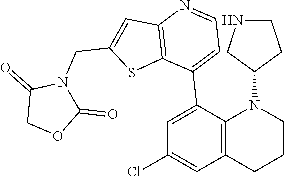 [SMILES: O=C1COC(=O)N1Cc1cc2nccc(-c3cc(Cl)cc4c3N([C@H]3CCNC3)CCC4)c2s1] |
| 74 | 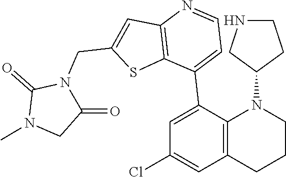 [SMILES: CN1CC(=O)N(Cc2cc3nccc(-c4cc(Cl)cc5c4N([C@H]4CCNC4)CCC5)c3s2)C1=O] |
| 75 | 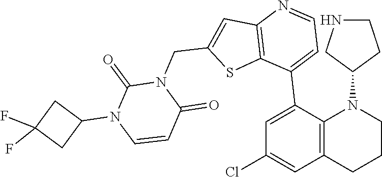 [SMILES: O=c1ccn(C2CC(F)(F)C2)c(=O)n1Cc1cc2nccc(-c3cc(Cl)cc4c3N([C@H]3CCNC3)CCC4)c2s1] |
| 76 | 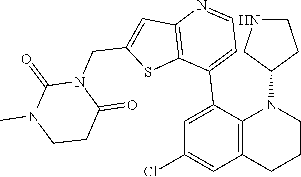 [SMILES: CN1CCC(=O)N(Cc2cc3nccc(-c4cc(Cl)cc5c4N([C@H]4CCNC4)CCC5)c3s2)C1=O] |
| 77 | 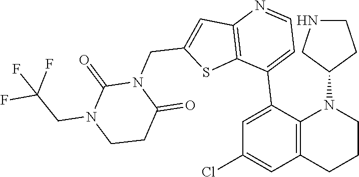 [SMILES: O=C1CCN(CC(F)(F)F)C(=O)N1Cc1cc2nccc(-c3cc(Cl)cc4c3N([C@H]3CCNC3)CCC4)c2s1] |
| 78 | 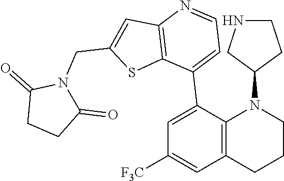 [SMILES: O=C1CCC(=O)N1Cc1cc2nccc(-c3cc(C(F)(F)F)cc4c3N([C@@H]3CCNC3)CCC4)c2s1] |
| 79 | 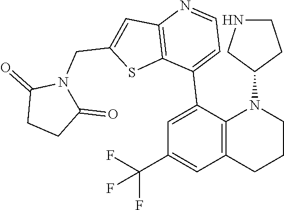 [SMILES: O=C1CCC(=O)N1Cc1cc2nccc(-c3cc(C(F)(F)F)cc4c3N([C@H]3CCNC3)CCC4)c2s1] |
| 80 | 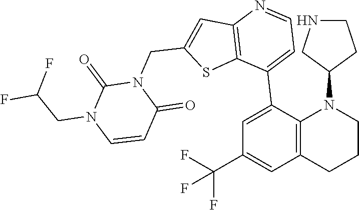 [SMILES: O=c1ccn(CC(F)F)c(=O)n1Cc1cc2nccc(-c3cc(C(F)(F)F)cc4c3N([C@@H]3CCNC3)CCC4)c2s1] |
| 81 | 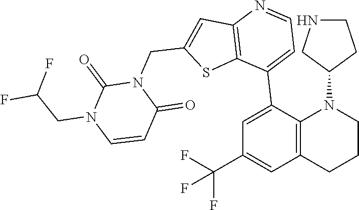 [SMILES: O=c1ccn(CC(F)F)c(=O)n1Cc1cc2nccc(-c3cc(C(F)(F)F)cc4c3N([C@H]3CCNC3)CCC4)c2s1] |
| 82 | 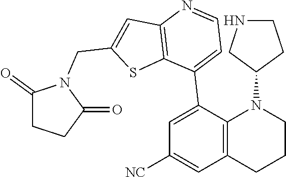 [SMILES: N#Cc1cc2c(c(-c3ccnc4cc(CN5C(=O)CCC5=O)sc34)c1)N([C@H]1CCNC1)CCC2] |
| 83 | 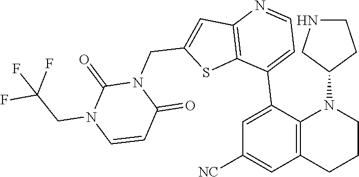 [SMILES: N#Cc1cc2c(c(-c3ccnc4cc(Cn5c(=O)ccn(CC(F)(F)F)c5=O)sc34)c1)N([C@H]1CCNC1)CCC2] |
| 84 | 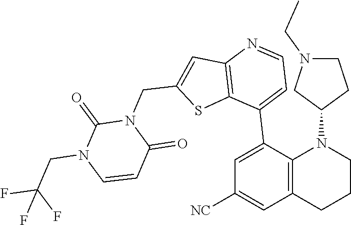 [SMILES: CCN1CC[C@H](N2CCCc3cc(C#N)cc(-c4ccnc5cc(Cn6c(=O)ccn(CC(F)(F)F)c6=O)sc45)c32)C1] |
| 85 | 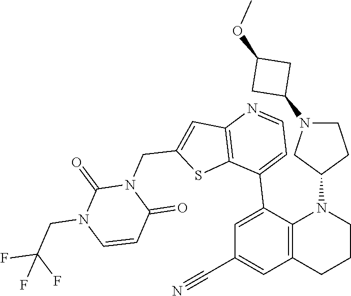 [SMILES: CO[C@H]1C[C@@H](N2CC[C@H](N3CCCc4cc(C#N)cc(-c5ccnc6cc(Cn7c(=O)ccn(CC(F)(F)F)c7=O)sc56)c43)C2)C1] |
| 86 | 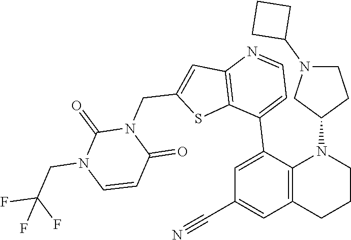 [SMILES: N#Cc1cc2c(c(-c3ccnc4cc(Cn5c(=O)ccn(CC(F)(F)F)c5=O)sc34)c1)N([C@H]1CCN(C3CCC3)C1)CCC2] |
| 87 | 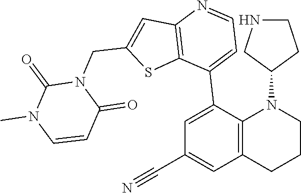 [SMILES: Cn1ccc(=O)n(Cc2cc3nccc(-c4cc(C#N)cc5c4N([C@H]4CCNC4)CCC5)c3s2)c1=O] |
| 88 | 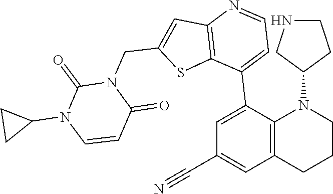 [SMILES: N#Cc1cc2c(c(-c3ccnc4cc(Cn5c(=O)ccn(C6CC6)c5=O)sc34)c1)N([C@H]1CCNC1)CCC2] |
| 89 | 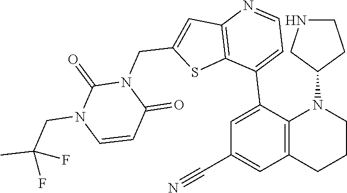 [SMILES: CC(F)(F)Cn1ccc(=O)n(Cc2cc3nccc(-c4cc(C#N)cc5c4N([C@H]4CCNC4)CCC5)c3s2)c1=O] |
| 90 | 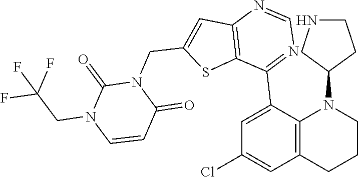 [SMILES: O=c1ccn(CC(F)(F)F)c(=O)n1Cc1cc2ncnc(-c3cc(Cl)cc4c3N([C@@H]3CCNC3)CCC4)c2s1] |
| 91 | 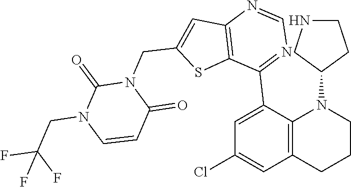 [SMILES: O=c1ccn(CC(F)(F)F)c(=O)n1Cc1cc2ncnc(-c3cc(Cl)cc4c3N([C@H]3CCNC3)CCC4)c2s1] |
| 92 | 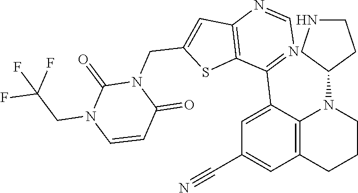 [SMILES: N#Cc1cc2c(c(-c3ncnc4cc(Cn5c(=O)ccn(CC(F)(F)F)c5=O)sc34)c1)N([C@H]1CCNC1)CCC2] |
| 93 | 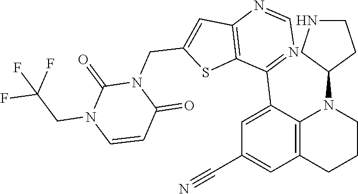 [SMILES: N#Cc1cc2c(c(-c3ncnc4cc(Cn5c(=O)ccn(CC(F)(F)F)c5=O)sc34)c1)N([C@@H]1CCNC1)CCC2] |
| 94 | 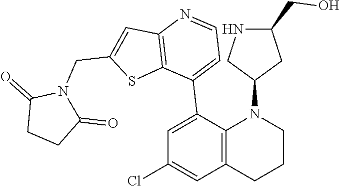 [SMILES: O=C1CCC(=O)N1Cc1cc2nccc(-c3cc(Cl)cc4c3N([C@H]3CN[C@@H](CO)C3)CCC4)c2s1] |
| 95a 95b | 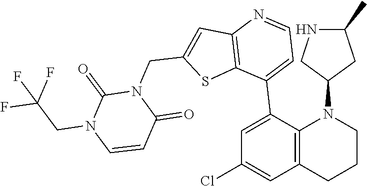 [SMILES: C[C@H]1C[C@@H](N2CCCc3cc(Cl)cc(-c4ccnc5cc(Cn6c(=O)ccn(CC(F)(F)F)c6=O)sc45)c32)CN1] |
| | 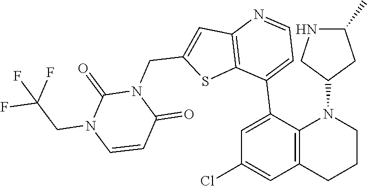 |
| 96a 96b | 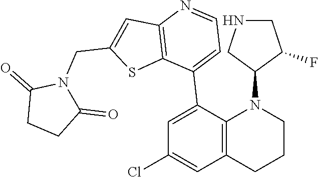 [SMILES: O=C1CCC(=O)N1Cc1cc2nccc(-c3cc(Cl)cc4c3N([C@H]3CNC[C@@H]3F)CCC4)c2s1] |
| | 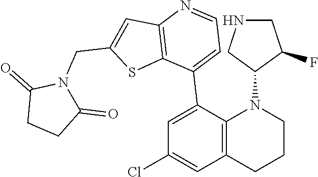 [SMILES: O=C1CCC(=O)N1Cc1cc2nccc(-c3cc(Cl)cc4c3N([C@@H]3CNC[C@H]3F)CCC4)c2s1] |
| 97a 97b | 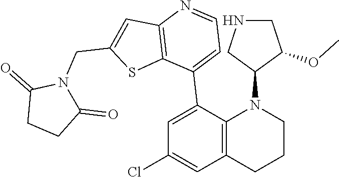 [SMILES: CO[C@H]1CNC[C@@H]1N1CCCc2cc(Cl)cc(-c3ccnc4cc(CN5C(=O)CCC5=O)sc34)c21] |
| | 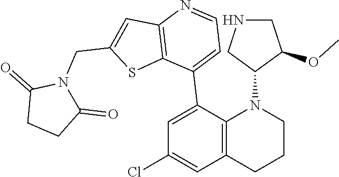 [SMILES: CO[C@@H]1CNC[C@H]1N1CCCc2cc(Cl)cc(-c3ccnc4cc(CN5C(=O)CCC5=O)sc34)c21] |
| 98 | 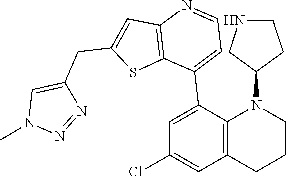 [SMILES: Cn1cc(Cc2cc3nccc(-c4cc(Cl)cc5c4N([C@@H]4CCNC4)CCC5)c3s2)nn1] |
| 99 | 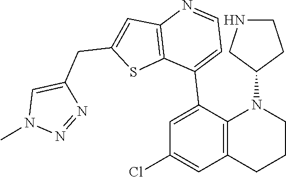 [SMILES: Cn1cc(Cc2cc3nccc(-c4cc(Cl)cc5c4N([C@H]4CCNC4)CCC5)c3s2)nn1] |
| 100 | 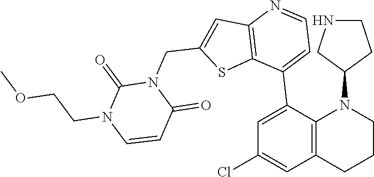 [SMILES: COCCn1ccc(=O)n(Cc2cc3nccc(-c4cc(Cl)cc5c4N([C@@H]4CCNC4)CCC5)c3s2)c1=O] |
| 101 | 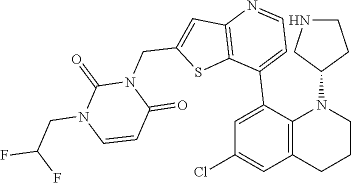 [SMILES: O=c1ccn(CC(F)F)c(=O)n1Cc1cc2nccc(-c3cc(Cl)cc4c3N([C@H]3CCNC3)CCC4)c2s1] |
| 102 | 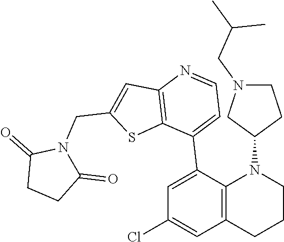 [SMILES: CC(C)CN1CC[C@H](N2CCCc3cc(Cl)cc(-c4ccnc5cc(CN6C(=O)CCC6=O)sc45)c32)C1] |
| 103 | 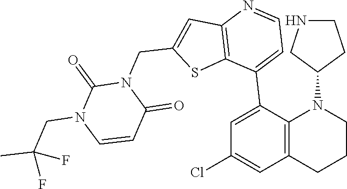 [SMILES: CC(F)(F)Cn1ccc(=O)n(Cc2cc3nccc(-c4cc(Cl)cc5c4N([C@H]4CCNC4)CCC5)c3s2)c1=O] |
| 104 | 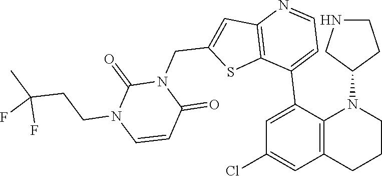 [SMILES: CC(F)(F)CCn1ccc(=O)n(Cc2cc3nccc(-c4cc(Cl)cc5c4N([C@H]4CCNC4)CCC5)c3s2)c1=O] |
| 105 | 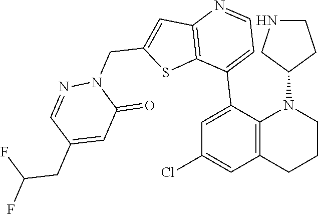 [SMILES: O=c1cc(CC(F)F)cnn1Cc1cc2nccc(-c3cc(Cl)cc4c3N([C@H]3CCNC3)CCC4)c2s1] |
| 106 | 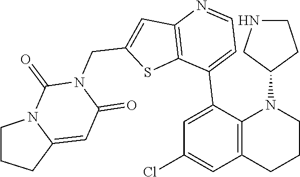 [SMILES: O=c1cc2n(c(=O)n1Cc1cc3nccc(-c4cc(Cl)cc5c4N([C@H]4CCNC4)CCC5)c3s1)CCC2] |
| 107a 107b | 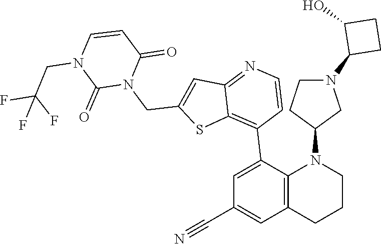 [SMILES: N#Cc1cc2c(c(-c3ccnc4cc(Cn5c(=O)ccn(CC(F)(F)F)c5=O)sc34)c1)N([C@H]1CCN([C@@H]3CC[C@H]3O)C1)CCC2] |
| | 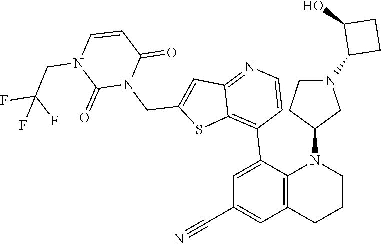 [SMILES: N#Cc1cc2c(c(-c3ccnc4cc(Cn5c(=O)ccn(CC(F)(F)F)c5=O)sc34)c1)N([C@H]1CCN([C@H]3CC[C@@H]3O)C1)CCC2] |
| 108a | 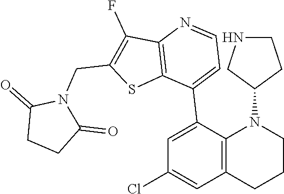 [SMILES: O=C1CCC(=O)N1Cc1sc2c(-c3cc(Cl)cc4c3N([C@H]3CCNC3)CCC4)ccnc2c1F] |
| 108b | 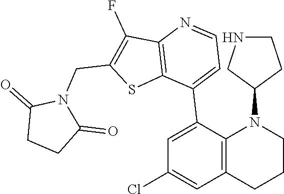 [SMILES: O=C1CCC(=O)N1Cc1sc2c(-c3cc(Cl)cc4c3N([C@@H]3CCNC3)CCC4)ccnc2c1F] |
| 109 | 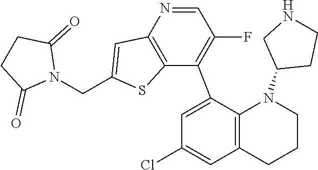 [SMILES: O=C1CCC(=O)N1Cc1cc2ncc(F)c(-c3cc(Cl)cc4c3N([C@H]3CCNC3)CCC4)c2s1] |
| 110 | 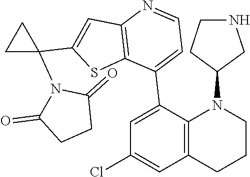 [SMILES: O=C1CCC(=O)N1C1(c2cc3nccc(-c4cc(Cl)cc5c4N([C@H]4CCNC4)CCC5)c3s2)CC1] |
| 111a | 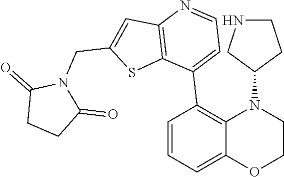 [SMILES: O=C1CCC(=O)N1Cc1cc2nccc(-c3cccc4c3N([C@H]3CCNC3)CCO4)c2s1] |
| 111b | 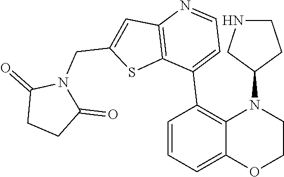 [SMILES: O=C1CCC(=O)N1Cc1cc2nccc(-c3cccc4c3N([C@@H]3CCNC3)CCO4)c2s1] |
| 112a | 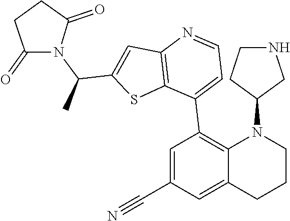 [SMILES: C[C@H](c1cc2nccc(-c3cc(C#N)cc4c3N([C@H]3CCNC3)CCC4)c2s1)N1C(=O)CCC1=O] |
| 112b | 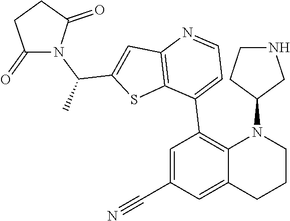 [SMILES: C[C@@H](c1cc2nccc(-c3cc(C#N)cc4c3N([C@H]3CCNC3)CCC4)c2s1)N1C(=O)CCC1=O] |
| 112c | 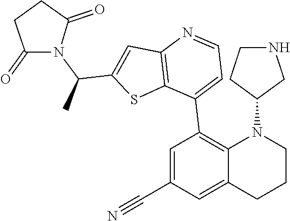 [SMILES: C[C@H](c1cc2nccc(-c3cc(C#N)cc4c3N([C@@H]3CCNC3)CCC4)c2s1)N1C(=O)CCC1=O] |
| 112d | 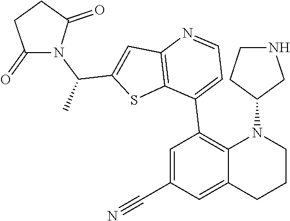 [SMILES: C[C@@H](c1cc2nccc(-c3cc(C#N)cc4c3N([C@@H]3CCNC3)CCC4)c2s1)N1C(=O)CCC1=O] |
| 113a | 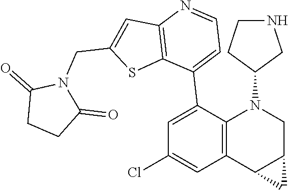 [SMILES: O=C1CCC(=O)N1Cc1cc2nccc(-c3cc(Cl)cc4c3N([C@@H]3CCNC3)C[C@@H]3C[C@H]43)c2s1] |
| 113b | 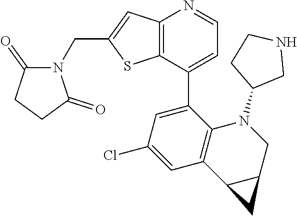 [SMILES: O=C1CCC(=O)N1Cc1cc2nccc(-c3cc(Cl)cc4c3N([C@@H]3CCNC3)C[C@H]3C[C@@H]43)c2s1] |
| 113c | 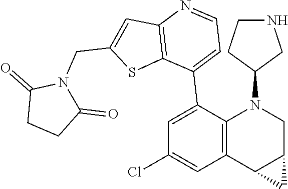 [SMILES: O=C1CCC(=O)N1Cc1cc2nccc(-c3cc(Cl)cc4c3N([C@H]3CCNC3)C[C@@H]3C[C@H]43)c2s1] |
| 113d | 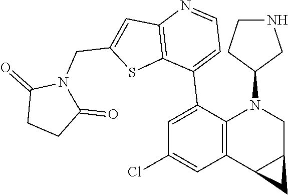 [SMILES: O=C1CCC(=O)N1Cc1cc2nccc(-c3cc(Cl)cc4c3N([C@H]3CCNC3)C[C@H]3C[C@@H]43)c2s1] |
| 114 | 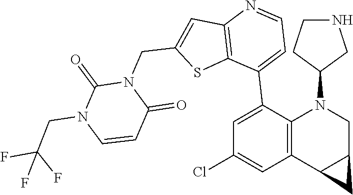 [SMILES: O=c1ccn(CC(F)(F)F)c(=O)n1Cc1cc2nccc(-c3cc(Cl)cc4c3N([C@H]3CCNC3)C[C@H]3C[C@@H]43)c2s1] |
| 115 | 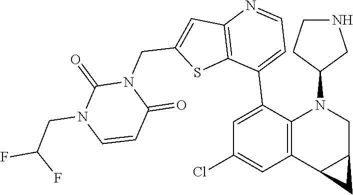 [SMILES: O=c1ccn(CC(F)F)c(=O)n1Cc1cc2nccc(-c3cc(Cl)cc4c3N([C@H]3CCNC3)C[C@H]3C[C@@H]43)c2s1] |
| 116 | 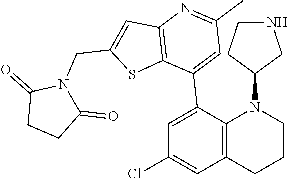 [SMILES: Cc1cc(-c2cc(Cl)cc3c2N([C@H]2CCNC2)CCC3)c2sc(CN3C(=O)CCC3=O)cc2n1] |
| 117a | 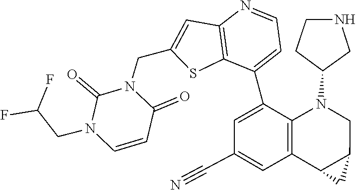 [SMILES: N#Cc1cc(-c2ccnc3cc(Cn4c(=O)ccn(CC(F)F)c4=O)sc23)c2c(c1)[C@H]1C[C@H]1CN2[C@@H]1CCNC1] |
| 117b | 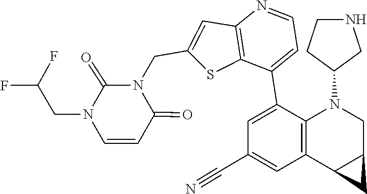 [SMILES: N#Cc1cc(-c2ccnc3cc(Cn4c(=O)ccn(CC(F)F)c4=O)sc23)c2c(c1)[C@@H]1C[C@@H]1CN2[C@@H]1CCNC1] |
| 117c | 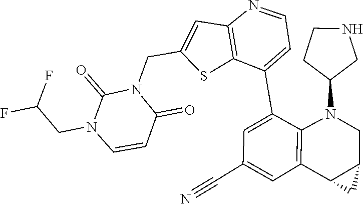 [SMILES: N#Cc1cc(-c2ccnc3cc(Cn4c(=O)ccn(CC(F)F)c4=O)sc23)c2c(c1)[C@H]1C[C@H]1CN2[C@H]1CCNC1] |
| 117d | 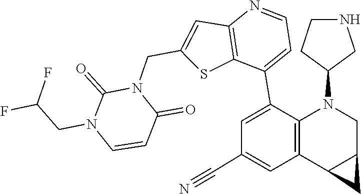 [SMILES: N#Cc1cc(-c2ccnc3cc(Cn4c(=O)ccn(CC(F)F)c4=O)sc23)c2c(c1)[C@@H]1C[C@@H]1CN2[C@H]1CCNC1] |
| 118a | 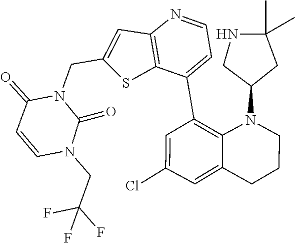 [SMILES: CC1(C)C[C@@H](N2CCCc3cc(Cl)cc(-c4ccnc5cc(Cn6c(=O)ccn(CC(F)(F)F)c6=O)sc45)c32)CN1] |
| 118b | 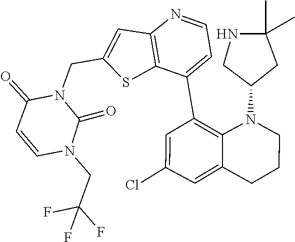 [SMILES: CC1(C)C[C@H](N2CCCc3cc(Cl)cc(-c4ccnc5cc(Cn6c(=O)ccn(CC(F)(F)F)c6=O)sc45)c32)CN1] |
| 119 | 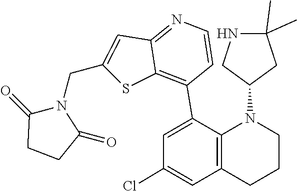 [SMILES: CC1(C)C[C@H](N2CCCc3cc(Cl)cc(-c4ccnc5cc(CN6C(=O)CCC6=O)sc45)c32)CN1] |
| 120a | 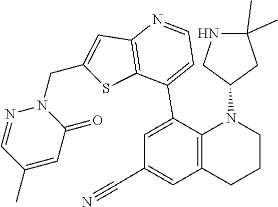 [SMILES: Cc1cnn(Cc2cc3nccc(-c4cc(C#N)cc5c4N([C@@H]4CNC(C)(C)C4)CCC5)c3s2)c(=O)c1] |
| 120b | 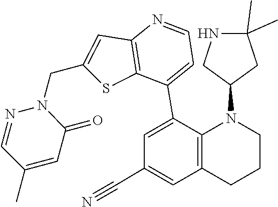 [SMILES: Cc1cnn(Cc2cc3nccc(-c4cc(C#N)cc5c4N([C@H]4CNC(C)(C)C4)CCC5)c3s2)c(=O)c1] |
| 121 | 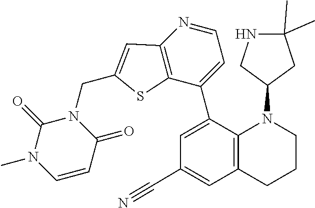 |
| 122 | 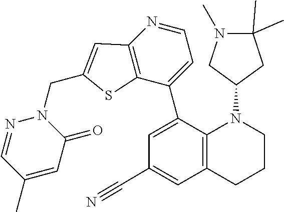 [SMILES: Cc1cnn(Cc2cc3nccc(-c4cc(C#N)cc5c4N([C@@H]4CN(C)C(C)(C)C4)CCC5)c3s2)c(=O)c1] |
| 123 | 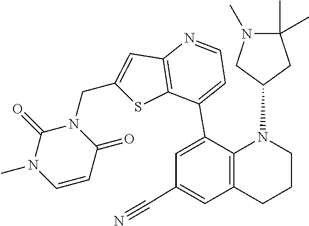 |
| 124 | 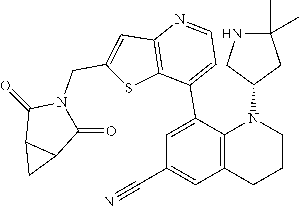 [SMILES: CC1(C)C[C@H](N2CCCc3cc(C#N)cc(-c4ccnc5cc(CN6C(=O)C7CC7C6=O)sc45)c32)CN1] |
| 125 | 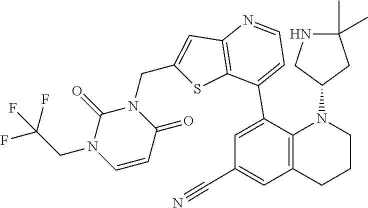 [SMILES: CC1(C)C[C@H](N2CCCc3cc(C#N)cc(-c4ccnc5cc(Cn6c(=O)ccn(CC(F)(F)F)c6=O)sc45)c32)CN1] |
| 126 | 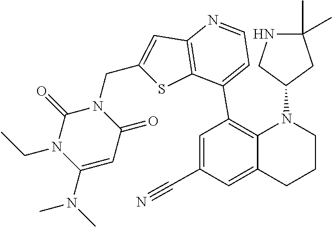 [SMILES: CCn1c(N(C)C)cc(=O)n(Cc2cc3nccc(-c4cc(C#N)cc5c4N([C@@H]4CNC(C)(C)C4)CCC5)c3s2)c1=O] |
| 127 | 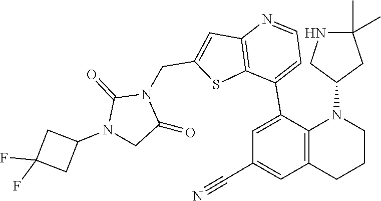 [SMILES: CC1(C)C[C@H](N2CCCc3cc(C#N)cc(-c4ccnc5cc(CN6C(=O)CN(C7CC(F)(F)C7)C6=O)sc45)c32)CN1] |
| 128 | 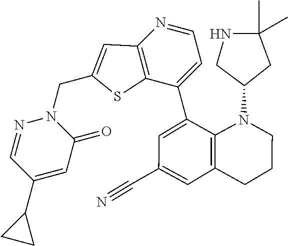 [SMILES: CC1(C)C[C@H](N2CCCc3cc(C#N)cc(-c4ccnc5cc(Cn6ncc(C7CC7)cc6=O)sc45)c32)CN1] |
| 129 | 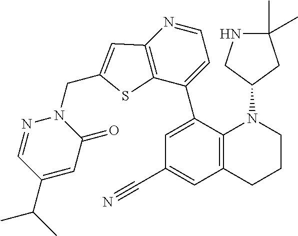 [SMILES: CC(C)c1cnn(Cc2cc3nccc(-c4cc(C#N)cc5c4N([C@@H]4CNC(C)(C)C4)CCC5)c3s2)c(=O)c1] |
| 130 | 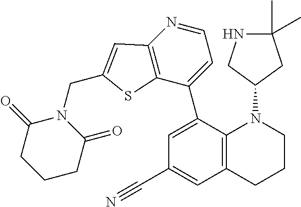 [SMILES: CC1(C)C[C@H](N2CCCc3cc(C#N)cc(-c4ccnc5cc(CN6C(=O)CCCC6=O)sc45)c32)CN1] |
| 131 | 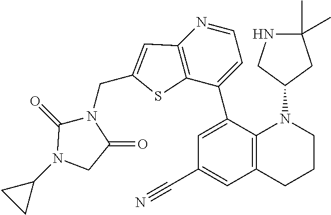 [SMILES: CC1(C)C[C@H](N2CCCc3cc(C#N)cc(-c4ccnc5cc(CN6C(=O)CN(C7CC7)C6=O)sc45)c32)CN1] |
| 132 | 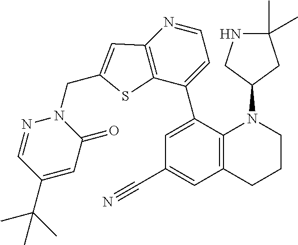 [SMILES: CC1(C)C[C@@H](N2CCCc3cc(C#N)cc(-c4ccnc5cc(Cn6ncc(C(C)(C)C)cc6=O)sc45)c32)CN1] |
| 133a | 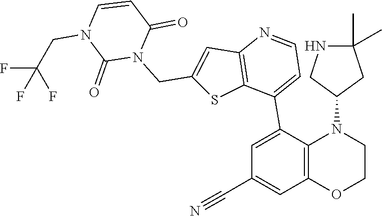 [SMILES: CC1(C)C[C@H](N2CCOc3cc(C#N)cc(-c4ccnc5cc(Cn6c(=O)ccn(CC(F)(F)F)c6=O)sc45)c32)CN1] |
| 133b | 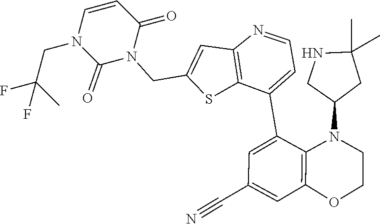 [SMILES: CC(F)(F)Cn1ccc(=O)n(Cc2cc3nccc(-c4cc(C#N)cc5c4N([C@H]4CNC(C)(C)C4)CCO5)c3s2)c1=O] |
| 134 | 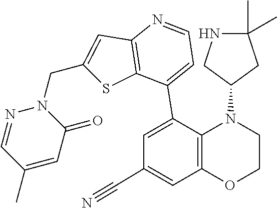 [SMILES: Cc1cnn(Cc2cc3nccc(-c4cc(C#N)cc5c4N([C@@H]4CNC(C)(C)C4)CCO5)c3s2)c(=O)c1] |
| 135 | 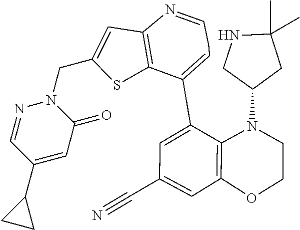 [SMILES: CC1(C)C[C@H](N2CCOc3cc(C#N)cc(C4=CCC=Nc5cc(Cn6ncc(C7CC7)cc6=O)sc54)c32)CN1] |
| 136 | 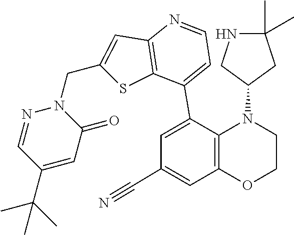 [SMILES: CC1(C)C[C@H](N2CCOc3cc(C#N)cc(-c4ccnc5cc(Cn6ncc(C(C)(C)C)cc6=O)sc45)c32)CN1] |
| 137 | 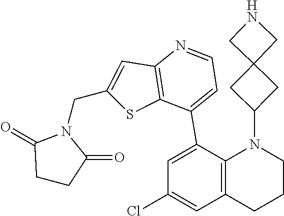 [SMILES: O=C1CCC(=O)N1Cc1cc2nccc(-c3cc(Cl)cc4c3N(C3CC5(CNC5)C3)CCC4)c2s1] |
| 138 | 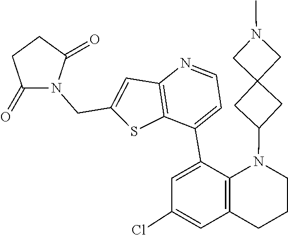 [SMILES: CN1CC2(CC(N3CCCc4cc(Cl)cc(-c5ccnc6cc(CN7C(=O)CCC7=O)sc56)c43)C2)C1] |
| 139a | 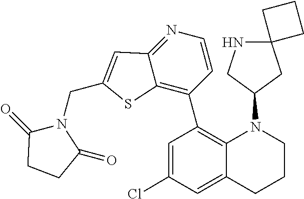 [SMILES: O=C1CCC(=O)N1Cc1cc2nccc(-c3cc(Cl)cc4c3N([C@H]3CNC5(CCC5)C3)CCC4)c2s1] |
| 139b | 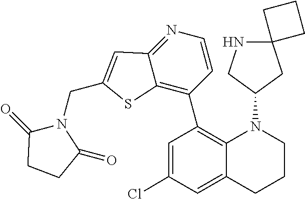 [SMILES: O=C1CCC(=O)N1Cc1cc2nccc(-c3cc(Cl)cc4c3N([C@@H]3CNC5(CCC5)C3)CCC4)c2s1] |
| 140 | 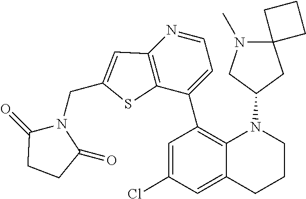 [SMILES: CN1C[C@@H](N2CCCc3cc(Cl)cc(-c4ccnc5cc(CN6C(=O)CCC6=O)sc45)c32)CC12CCC2] |
| 141 | 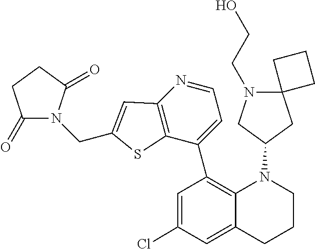 [SMILES: O=C1CCC(=O)N1Cc1cc2nccc(-c3cc(Cl)cc4c3N([C@@H]3CN(CCO)C5(CCC5)C3)CCC4)c2s1] |
| 142 | 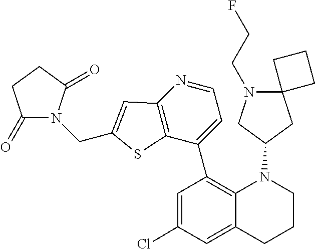 [SMILES: O=C1CCC(=O)N1Cc1cc2nccc(-c3cc(Cl)cc4c3N([C@@H]3CN(CCF)C5(CCC5)C3)CCC4)c2s1] |
| 143a | 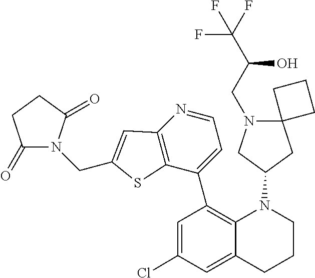 [SMILES: O=C1CCC(=O)N1Cc1cc2nccc(-c3cc(Cl)cc4c3N([C@@H]3CN(C[C@H](O)C(F)(F)F)C5(CCC5)C3)CCC4)c2s1] |
| 143b | 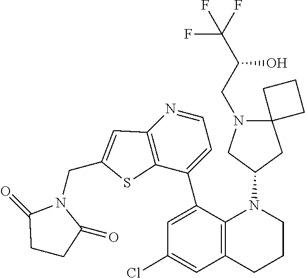 [SMILES: O=C1CCC(=O)N1Cc1cc2nccc(-c3cc(Cl)cc4c3N([C@@H]3CN(C[C@@H](O)C(F)(F)F)C5(CCC5)C3)CCC4)c2s1] |
| 144 | 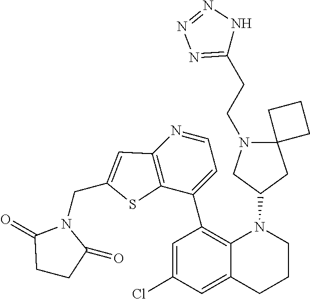 |
| 145 | 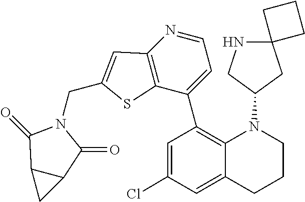 [SMILES: O=C1C2CC2C(=O)N1Cc1cc2nccc(-c3cc(Cl)cc4c3N([C@@H]3CNC5(CCC5)C3)CCC4)c2s1] |
| 146 | 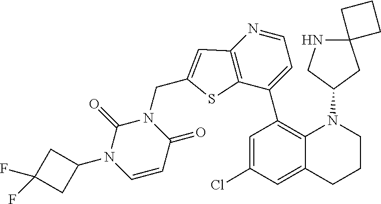 [SMILES: O=c1ccn(C2CC(F)(F)C2)c(=O)n1Cc1cc2nccc(-c3cc(Cl)cc4c3N([C@@H]3CNC5(CCC5)C3)CCC4)c2s1] |
| 147 | 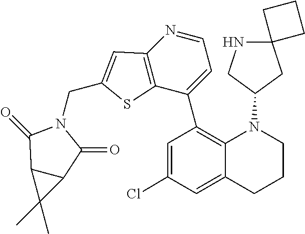 [SMILES: CC1(C)C2C(=O)N(Cc3cc4nccc(-c5cc(Cl)cc6c5N([C@@H]5CNC7(CCC7)C5)CCC6)c4s3)C(=O)C21] |
| 148 | 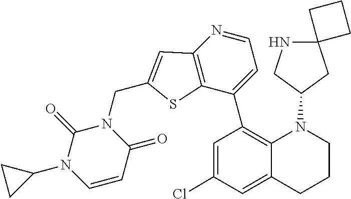 [SMILES: O=c1ccn(C2CC2)c(=O)n1Cc1cc2nccc(-c3cc(Cl)cc4c3N([C@@H]3CNC5(CCC5)C3)CCC4)c2s1] |
| 149 | 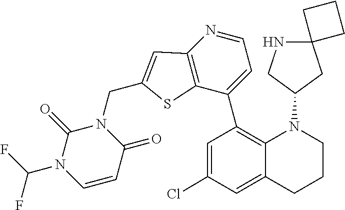 [SMILES: O=c1ccn(C(F)F)c(=O)n1Cc1cc2nccc(-c3cc(Cl)cc4c3N([C@@H]3CNC5(CCC5)C3)CCC4)c2s1] |
| 150 | 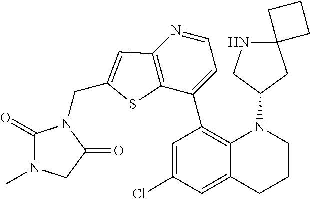 [SMILES: CN1CC(=O)N(Cc2cc3nccc(-c4cc(Cl)cc5c4N([C@@H]4CNC6(CCC6)C4)CCC5)c3s2)C1=O] |
| 151 | 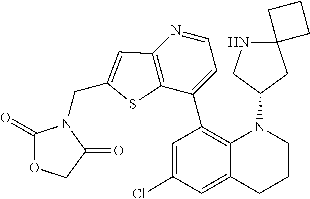 [SMILES: O=C1COC(=O)N1Cc1cc2nccc(-c3cc(Cl)cc4c3N([C@@H]3CNC5(CCC5)C3)CCC4)c2s1] |
| 152 | 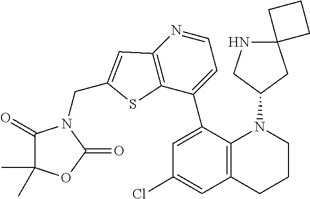 [SMILES: CC1(C)OC(=O)N(Cc2cc3nccc(-c4cc(Cl)cc5c4N([C@@H]4CNC6(CCC6)C4)CCC5)c3s2)C1=O] |
| 153 | 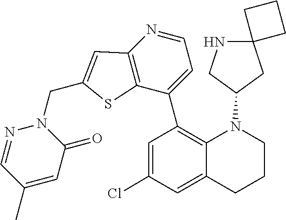 [SMILES: Cc1cnn(Cc2cc3nccc(-c4cc(Cl)cc5c4N([C@@H]4CNC6(CCC6)C4)CCC5)c3s2)c(=O)c1] |
| 154 | 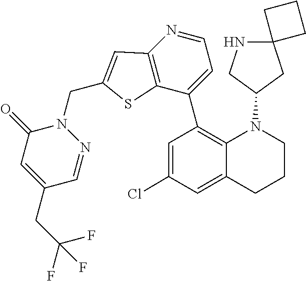 [SMILES: O=c1cc(CC(F)(F)F)cnn1Cc1cc2nccc(-c3cc(Cl)cc4c3N([C@@H]3CNC5(CCC5)C3)CCC4)c2s1] |
| 155 | 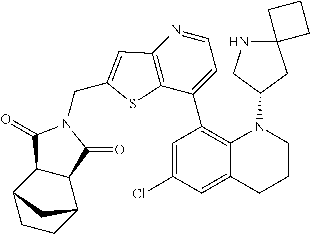 [SMILES: O=C1[C@@H]2[C@H]3CC[C@H](C3)[C@@H]2C(=O)N1Cc1cc2nccc(-c3cc(Cl)cc4c3N([C@@H]3CNC5(CCC5)C3)CCC4)c2s1] |
| 156 | 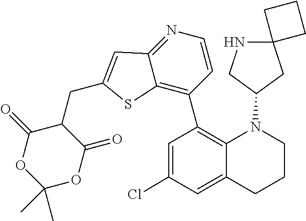 [SMILES: CC1(C)OC(=O)C(Cc2cc3nccc(-c4cc(Cl)cc5c4N([C@@H]4CNC6(CCC6)C4)CCC5)c3s2)C(=O)O1] |
| 157 | 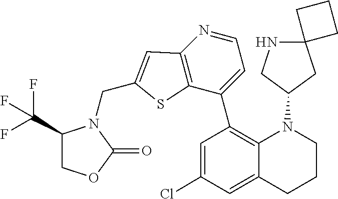 [SMILES: O=C1OC[C@@H](C(F)(F)F)N1Cc1cc2nccc(-c3cc(Cl)cc4c3N([C@@H]3CNC5(CCC5)C3)CCC4)c2s1] |
| 158 | 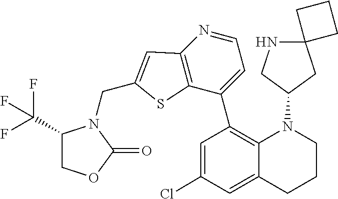 [SMILES: O=C1OC[C@H](C(F)(F)F)N1Cc1cc2nccc(-c3cc(Cl)cc4c3N([C@@H]3CNC5(CCC5)C3)CCC4)c2s1] |
| 159 | 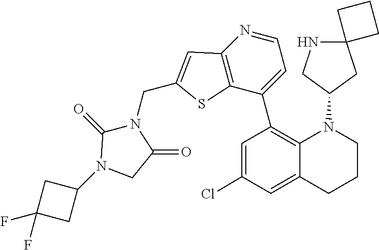 [SMILES: O=C1CN(C2CC(F)(F)C2)C(=O)N1Cc1cc2nccc(-c3cc(Cl)cc4c3N([C@@H]3CNC5(CCC5)C3)CCC4)c2s1] |
| 160 | 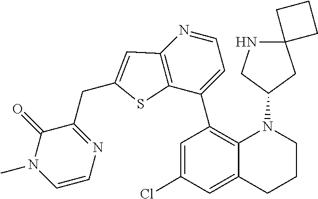 [SMILES: Cn1ccnc(Cc2cc3nccc(-c4cc(Cl)cc5c4N([C@@H]4CNC6(CCC6)C4)CCC5)c3s2)c1=O] |
| 161a | 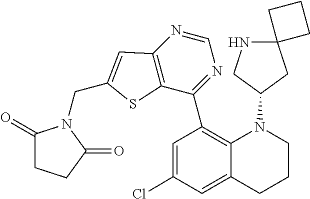 [SMILES: O=C1CCC(=O)N1Cc1cc2ncnc(-c3cc(Cl)cc4c3N([C@@H]3CNC5(CCC5)C3)CCC4)c2s1] |
| 161b | 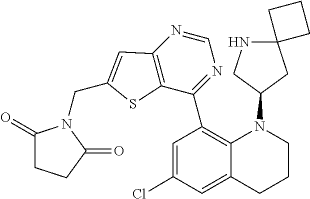 [SMILES: O=C1CCC(=O)N1Cc1cc2ncnc(-c3cc(Cl)cc4c3N([C@H]3CNC5(CCC5)C3)CCC4)c2s1] |
| 162a | 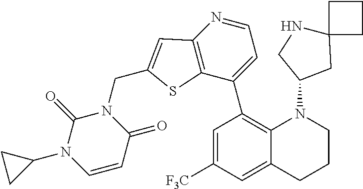 [SMILES: O=c1ccn(C2CC2)c(=O)n1Cc1cc2nccc(-c3cc(C(F)(F)F)cc4c3N([C@@H]3CNC5(CCC5)C3)CCC4)c2s1] |
| 162b | 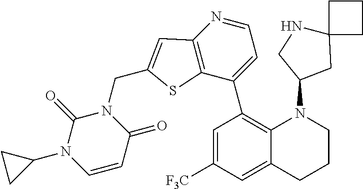 [SMILES: O=c1ccn(C2CC2)c(=O)n1Cc1cc2nccc(-c3cc(C(F)(F)F)cc4c3N([C@H]3CNC5(CCC5)C3)CCC4)c2s1] |
| 163 | 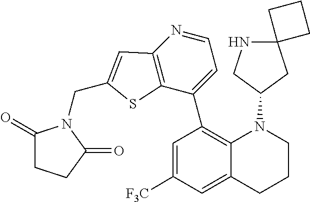 [SMILES: O=C1CCC(=O)N1Cc1cc2nccc(-c3cc(C(F)(F)F)cc4c3N([C@@H]3CNC5(CCC5)C3)CCC4)c2s1] |
| 164a | 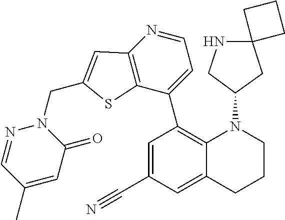 [SMILES: Cc1cnn(Cc2cc3nccc(-c4cc(C#N)cc5c4N([C@@H]4CNC6(CCC6)C4)CCC5)c3s2)c(=O)c1] |
| 164b | 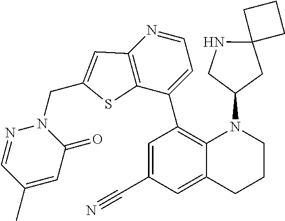 [SMILES: Cc1cnn(Cc2cc3nccc(-c4cc(C#N)cc5c4N([C@H]4CNC6(CCC6)C4)CCC5)c3s2)c(=O)c1] |
| 165a | 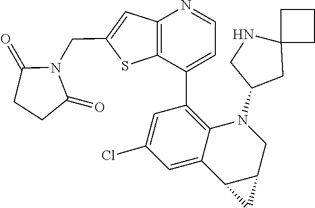 [SMILES: O=C1CCC(=O)N1Cc1cc2nccc(-c3cc(Cl)cc4c3N([C@@H]3CNC5(CCC5)C3)C[C@@H]3C[C@H]43)c2s1] |
| 165b | 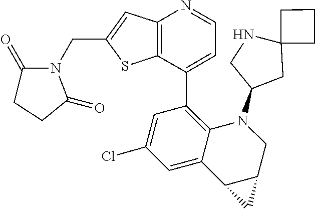 [SMILES: O=C1CCC(=O)N1Cc1cc2nccc(-c3cc(Cl)cc4c3N([C@H]3CNC5(CCC5)C3)C[C@@H]3C[C@H]43)c2s1] |
| 165c | 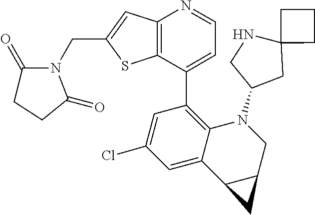 [SMILES: O=C1CCC(=O)N1Cc1cc2nccc(-c3cc(Cl)cc4c3N([C@@H]3CNC5(CCC5)C3)C[C@H]3C[C@@H]43)c2s1] |
| 165d | 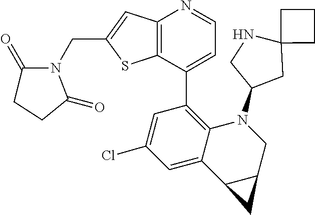 [SMILES: O=C1CCC(=O)N1Cc1cc2nccc(-c3cc(Cl)cc4c3N([C@H]3CNC5(CCC5)C3)C[C@H]3C[C@@H]43)c2s1] |
| 166 | 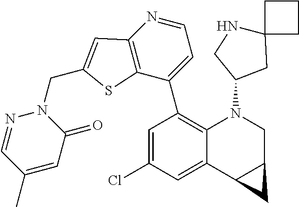 [SMILES: Cc1cnn(Cc2cc3nccc(-c4cc(Cl)cc5c4N([C@@H]4CNC6(CCC6)C4)C[C@H]4C[C@@H]54)c3s2)c(=O)c1] |
| 167a | 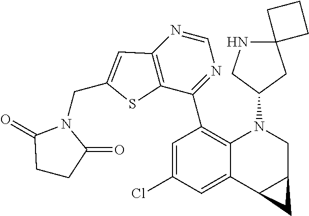 [SMILES: O=C1CCC(=O)N1Cc1cc2ncnc(-c3cc(Cl)cc4c3N([C@@H]3CNC5(CCC5)C3)C[C@H]3C[C@@H]43)c2s1] |
| 167b | 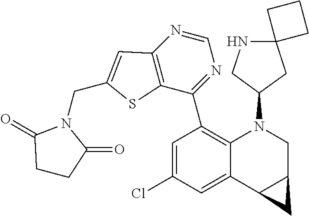 [SMILES: O=C1CCC(=O)N1Cc1cc2ncnc(-c3cc(Cl)cc4c3N([C@H]3CNC5(CCC5)C3)C[C@H]3C[C@@H]43)c2s1] |
| 167c | 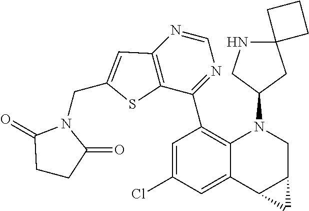 [SMILES: O=C1CCC(=O)N1Cc1cc2ncnc(-c3cc(Cl)cc4c3N([C@H]3CNC5(CCC5)C3)C[C@@H]3C[C@H]43)c2s1] |
| 167d | 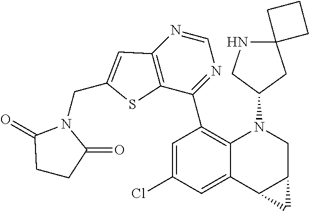 [SMILES: O=C1CCC(=O)N1Cc1cc2ncnc(-c3cc(Cl)cc4c3N([C@@H]3CNC5(CCC5)C3)C[C@@H]3C[C@H]43)c2s1] |
| 168 | 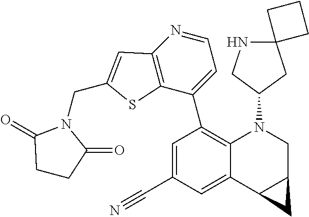 [SMILES: N#Cc1cc(-c2ccnc3cc(CN4C(=O)CCC4=O)sc23)c2c(c1)[C@@H]1C[C@@H]1CN2[C@@H]1CNC2(CCC2)C1] |
| 169 | 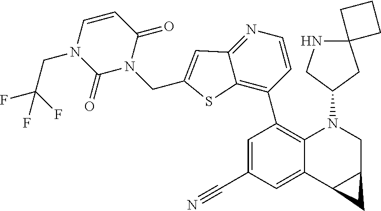 [SMILES: N#Cc1cc(-c2ccnc3cc(Cn4c(=O)ccn(CC(F)(F)F)c4=O)sc23)c2c(c1)[C@@H]1C[C@@H]1CN2[C@@H]1CNC2(CCC2)C1] |
| 170 | 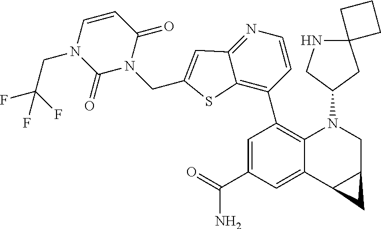 [SMILES: NC(=O)c1cc(-c2ccnc3cc(Cn4c(=O)ccn(CC(F)(F)F)c4=O)sc23)c2c(c1)[C@@H]1C[C@@H]1CN2[C@@H]1CNC2(CCC2)C1] |
| 171a | 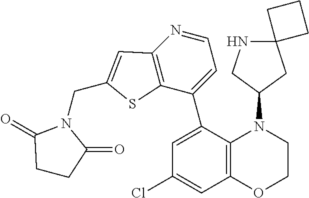 [SMILES: O=C1CCC(=O)N1Cc1cc2nccc(-c3cc(Cl)cc4c3N([C@H]3CNC5(CCC5)C3)CCO4)c2s1] |
| 171b | 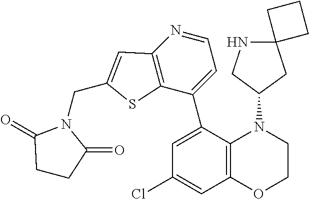 [SMILES: O=C1CCC(=O)N1Cc1cc2nccc(-c3cc(Cl)cc4c3N([C@@H]3CNC5(CCC5)C3)CCO4)c2s1] |
| 172 | 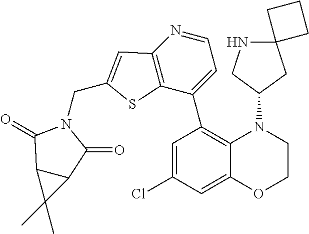 [SMILES: CC1(C)C2C(=O)N(Cc3cc4nccc(-c5cc(Cl)cc6c5N([C@@H]5CNC7(CCC7)C5)CCO6)c4s3)C(=O)C21] |
| 173a | 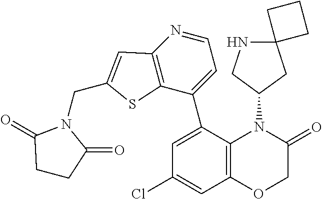 [SMILES: O=C1CCC(=O)N1Cc1cc2nccc(-c3cc(Cl)cc4c3N([C@@H]3CNC5(CCC5)C3)C(=O)CO4)c2s1] |
| 173b | 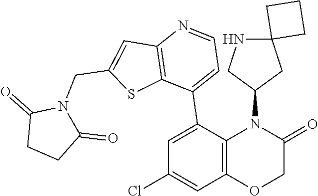 [SMILES: O=C1CCC(=O)N1Cc1cc2nccc(-c3cc(Cl)cc4c3N([C@H]3CNC5(CCC5)C3)C(=O)CO4)c2s1] |
| 174 | 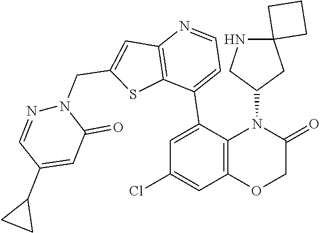 |
| 175a | 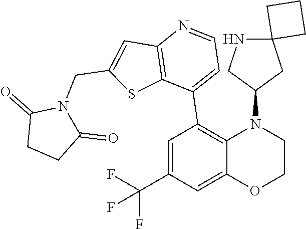 [SMILES: O=C1CCC(=O)N1Cc1cc2nccc(-c3cc(C(F)(F)F)cc4c3N([C@H]3CNC5(CCC5)C3)CCO4)c2s1] |
| 175b | 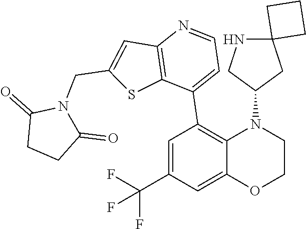 [SMILES: O=C1CCC(=O)N1Cc1cc2nccc(-c3cc(C(F)(F)F)cc4c3N([C@@H]3CNC5(CCC5)C3)CCO4)c2s1] |
| 176a | 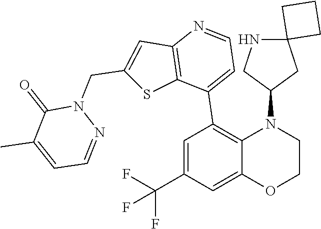 [SMILES: Cc1ccnn(Cc2cc3nccc(-c4cc(C(F)(F)F)cc5c4N([C@H]4CNC6(CCC6)C4)CCO5)c3s2)c1=O] |
| 176b | 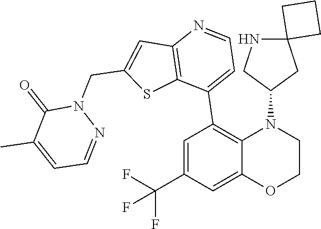 [SMILES: Cc1ccnn(Cc2cc3nccc(-c4cc(C(F)(F)F)cc5c4N([C@@H]4CNC6(CCC6)C4)CCO5)c3s2)c1=O] |
| 177a | 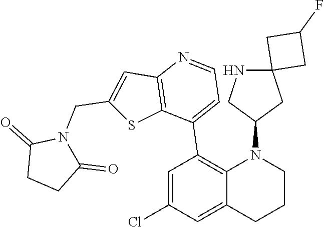 [SMILES: O=C1CCC(=O)N1Cc1cc2nccc(-c3cc(Cl)cc4c3N([C@H]3CNC5(CC(F)C5)C3)CCC4)c2s1] |
| 177b | 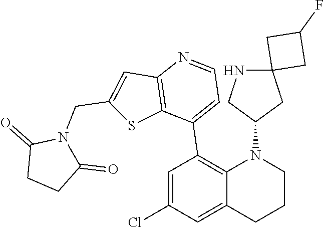 [SMILES: O=C1CCC(=O)N1Cc1cc2nccc(-c3cc(Cl)cc4c3N([C@@H]3CNC5(CC(F)C5)C3)CCC4)c2s1] |
| 178a | 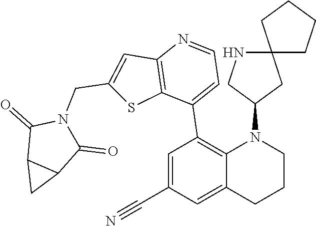 [SMILES: N#Cc1cc2c(c(-c3ccnc4cc(CN5C(=O)C6CC6C5=O)sc34)c1)N([C@H]1CNC3(CCCC3)C1)CCC2] |
| 178b | 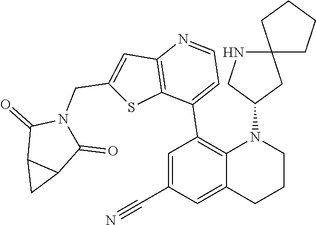 [SMILES: N#Cc1cc2c(c(-c3ccnc4cc(CN5C(=O)C6CC6C5=O)sc34)c1)N([C@@H]1CNC3(CCCC3)C1)CCC2] |
| 179a 179b | 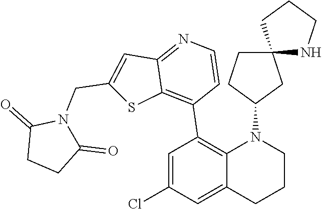 [SMILES: O=C1CCC(=O)N1Cc1cc2nccc(-c3cc(Cl)cc4c3N([C@@H]3CC[C@]5(CCCN5)C3)CCC4)c2s1] |
| | 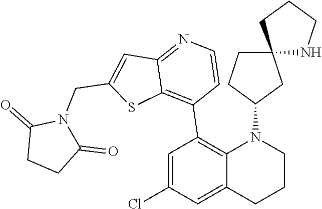 [SMILES: O=C1CCC(=O)N1Cc1cc2nccc(-c3cc(Cl)cc4c3N([C@@H]3CC[C@@]5(CCCN5)C3)CCC4)c2s1] |
| 180 | 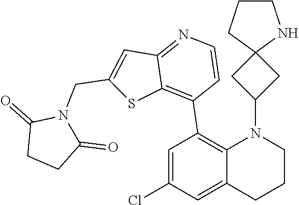 [SMILES: O=C1CCC(=O)N1Cc1cc2nccc(-c3cc(Cl)cc4c3N(C3CC5(CCCN5)C3)CCC4)c2s1] |
| 181a | 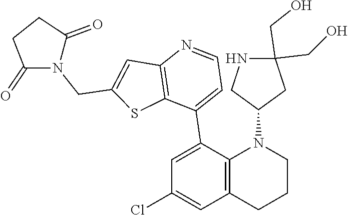 [SMILES: O=C1CCC(=O)N1Cc1cc2nccc(-c3cc(Cl)cc4c3N([C@@H]3CNC(CO)(CO)C3)CCC4)c2s1] |
| 181b | 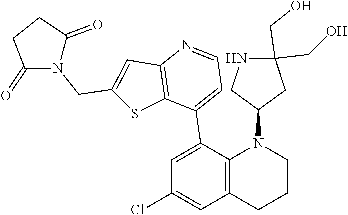 [SMILES: O=C1CCC(=O)N1Cc1cc2nccc(-c3cc(Cl)cc4c3N([C@H]3CNC(CO)(CO)C3)CCC4)c2s1] |
| 182 | 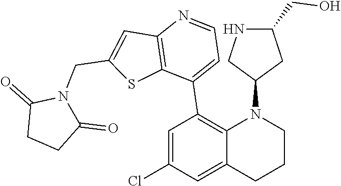 [SMILES: O=C1CCC(=O)N1Cc1cc2nccc(-c3cc(Cl)cc4c3N([C@H]3CN[C@H](CO)C3)CCC4)c2s1] |
| 183 | 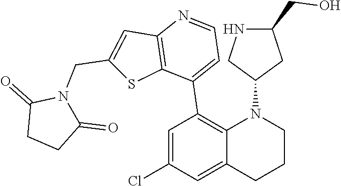 [SMILES: O=C1CCC(=O)N1Cc1cc2nccc(-c3cc(Cl)cc4c3N([C@@H]3CN[C@@H](CO)C3)CCC4)c2s1] |
| 184 | 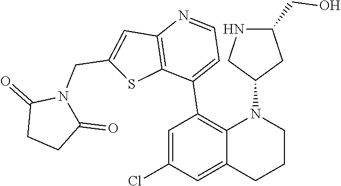 [SMILES: O=C1CCC(=O)N1Cc1cc2nccc(-c3cc(Cl)cc4c3N([C@@H]3CN[C@H](CO)C3)CCC4)c2s1] |
| 185 | 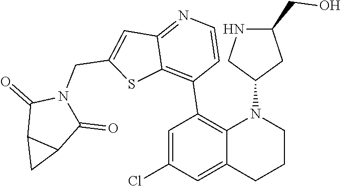 [SMILES: O=C1C2CC2C(=O)N1Cc1cc2nccc(-c3cc(Cl)cc4c3N([C@@H]3CN[C@@H](CO)C3)CCC4)c2s1] |
| 186 | 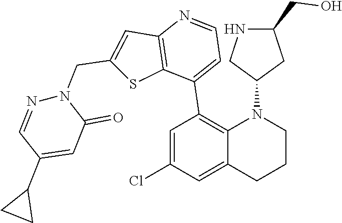 [SMILES: O=c1cc(C2CC2)cnn1Cc1cc2nccc(-c3cc(Cl)cc4c3N([C@@H]3CN[C@@H](CO)C3)CCC4)c2s1] |
| 187 | 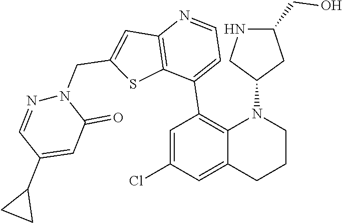 [SMILES: O=c1cc(C2CC2)cnn1Cc1cc2nccc(-c3cc(Cl)cc4c3N([C@@H]3CN[C@H](CO)C3)CCC4)c2s1] |
| 188 | 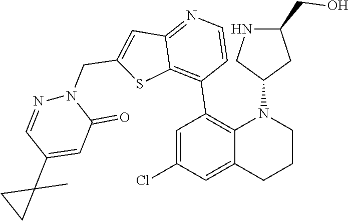 [SMILES: CC1(c2cnn(Cc3cc4nccc(-c5cc(Cl)cc6c5N([C@@H]5CN[C@@H](CO)C5)CCC6)c4s3)c(=O)c2)CC1] |
| 189 | 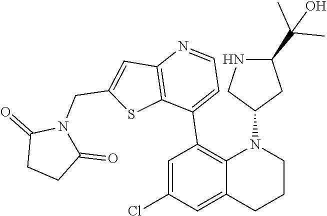 [SMILES: CC(C)(O)[C@H]1C[C@H](N2CCCc3cc(Cl)cc(-c4ccnc5cc(CN6C(=O)CCC6=O)sc45)c32)CN1] |
| 190 | 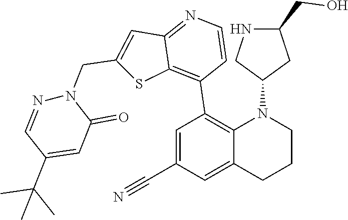 [SMILES: CC(C)(C)c1cnn(Cc2cc3nccc(-c4cc(C#N)cc5c4N([C@@H]4CN[C@@H](CO)C4)CCC5)c3s2)c(=O)c1] |
| 191 | 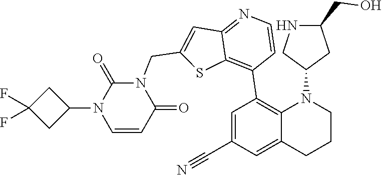 [SMILES: N#Cc1cc2c(c(-c3ccnc4cc(Cn5c(=O)ccn(C6CC(F)(F)C6)c5=O)sc34)c1)N([C@@H]1CN[C@@H](CO)C1)CCC2] |
| 192a 192b | 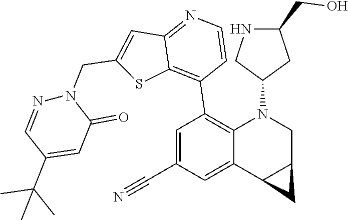 [SMILES: CC(C)(C)c1cnn(Cc2cc3nccc(-c4cc(C#N)cc5c4N([C@@H]4CN[C@@H](CO)C4)C[C@H]4C[C@@H]54)c3s2)c(=O)c1] |
| | 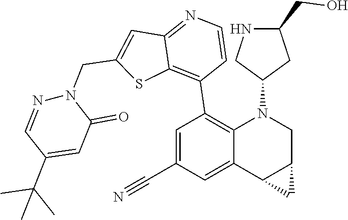 [SMILES: CC(C)(C)c1cnn(Cc2cc3nccc(-c4cc(C#N)cc5c4N([C@@H]4CN[C@@H](CO)C4)C[C@@H]4C[C@H]54)c3s2)c(=O)c1] |
| 193a 193b | 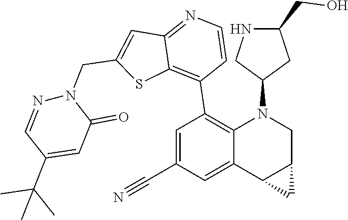 [SMILES: CC(C)(C)c1cnn(Cc2cc3nccc(-c4cc(C#N)cc5c4N([C@H]4CN[C@@H](CO)C4)C[C@@H]4C[C@H]54)c3s2)c(=O)c1] |
| | 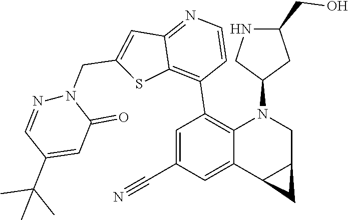 [SMILES: CC(C)(C)c1cnn(Cc2cc3nccc(-c4cc(C#N)cc5c4N([C@H]4CN[C@@H](CO)C4)C[C@H]4C[C@@H]54)c3s2)c(=O)c1] |
| 194a 194b | 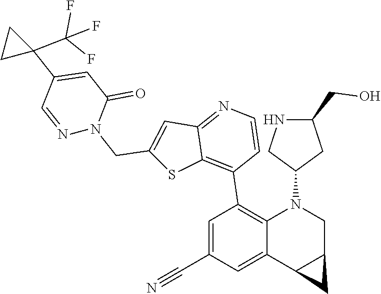 [SMILES: N#Cc1cc(-c2ccnc3cc(Cn4ncc(C5(C(F)(F)F)CC5)cc4=O)sc23)c2c(c1)[C@@H]1C[C@@H]1CN2[C@@H]1CN[C@@H](CO)C1] |
| | 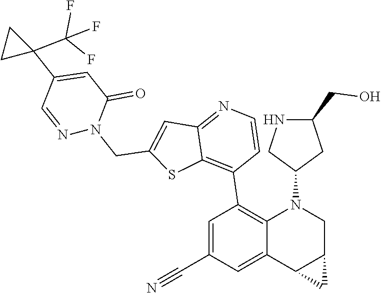 [SMILES: N#Cc1cc(-c2ccnc3cc(Cn4ncc(C5(C(F)(F)F)CC5)cc4=O)sc23)c2c(c1)[C@H]1C[C@H]1CN2[C@@H]1CN[C@@H](CO)C1] |
| 195a 195b | 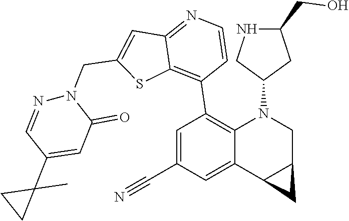 [SMILES: CC1(c2cnn(Cc3cc4nccc(-c5cc(C#N)cc6c5N([C@@H]5CN[C@@H](CO)C5)C[C@H]5C[C@@H]65)c4s3)c(=O)c2)CC1] |
| | 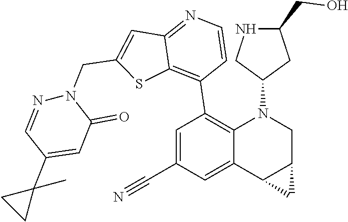 [SMILES: CC1(c2cnn(Cc3cc4nccc(-c5cc(C#N)cc6c5N([C@@H]5CN[C@@H](CO)C5)C[C@@H]5C[C@H]65)c4s3)c(=O)c2)CC1] |
| 196 | 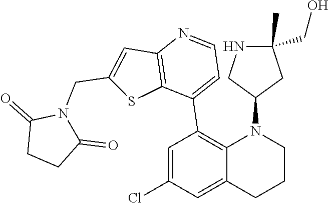 [SMILES: C[C@@]1(CO)C[C@@H](N2CCCc3cc(Cl)cc(-c4ccnc5cc(CN6C(=O)CCC6=O)sc45)c32)CN1] |
| 197 | 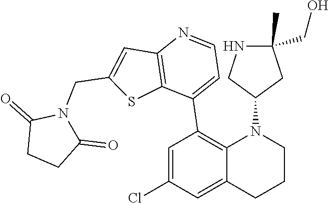 [SMILES: C[C@@]1(CO)C[C@H](N2CCCc3cc(Cl)cc(-c4ccnc5cc(CN6C(=O)CCC6=O)sc45)c32)CN1] |
| 198 | 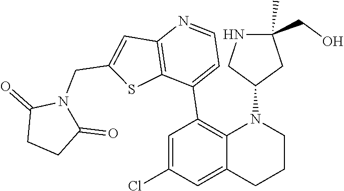 [SMILES: C[C@]1(CO)C[C@H](N2CCCc3cc(Cl)cc(-c4ccnc5cc(CN6C(=O)CCC6=O)sc45)c32)CN1] |
| 199 | 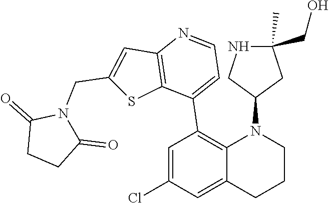 [SMILES: C[C@]1(CO)C[C@@H](N2CCCc3cc(Cl)cc(-c4ccnc5cc(CN6C(=O)CCC6=O)sc45)c32)CN1] |
| 200 | 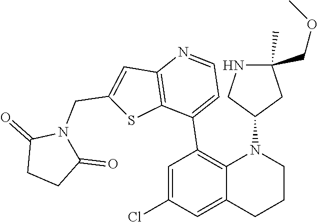 [SMILES: COC[C@@]1(C)C[C@H](N2CCCc3cc(Cl)cc(-c4ccnc5cc(CN6C(=O)CCC6=O)sc45)c32)CN1] |
| 201 | 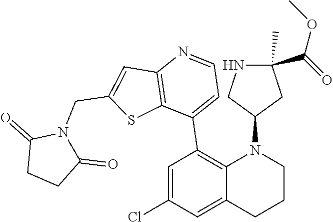 [SMILES: COC(=O)[C@@]1(C)C[C@@H](N2CCCc3cc(Cl)cc(-c4ccnc5cc(CN6C(=O)CCC6=O)sc45)c32)CN1] |
| 202 | 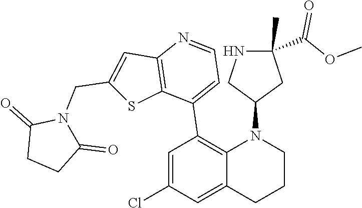 [SMILES: COC(=O)[C@]1(C)C[C@@H](N2CCCc3cc(Cl)cc(-c4ccnc5cc(CN6C(=O)CCC6=O)sc45)c32)CN1] |
| 203 | 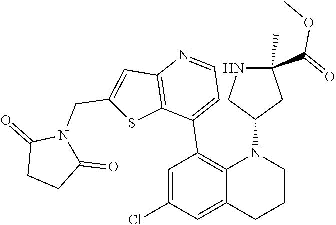 [SMILES: COC(=O)[C@@]1(C)C[C@H](N2CCCc3cc(Cl)cc(-c4ccnc5cc(CN6C(=O)CCC6=O)sc45)c32)CN1] |
| 204 | 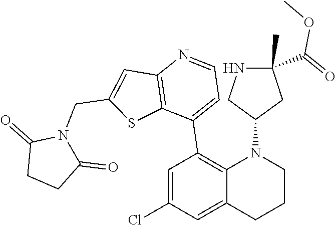 [SMILES: COC(=O)[C@]1(C)C[C@H](N2CCCc3cc(Cl)cc(-c4ccnc5cc(CN6C(=O)CCC6=O)sc45)c32)CN1] |
| 205 | 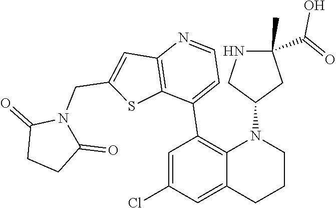 [SMILES: C[C@@]1(C(=O)O)C[C@H](N2CCCc3cc(Cl)cc(-c4ccnc5cc(CN6C(=O)CCC6=O)sc45)c32)CN1] |
| 206 | 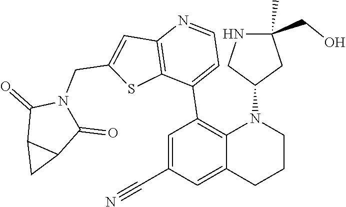 [SMILES: C[C@]1(CO)C[C@H](N2CCCc3cc(C#N)cc(-c4ccnc5cc(CN6C(=O)C7CC7C6=O)sc45)c32)CN1] |
| 207 | 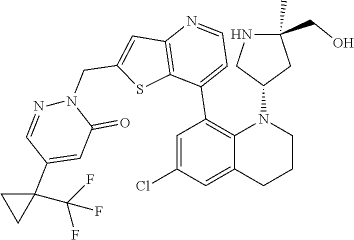 [SMILES: C[C@]1(CO)C[C@H](N2CCCc3cc(Cl)cc(-c4ccnc5cc(Cn6ncc(C7(C(F)(F)F)CC7)cc6=O)sc45)c32)CN1] |
| 208 | 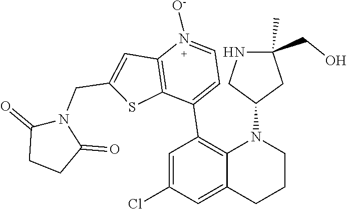 |
| 209 | 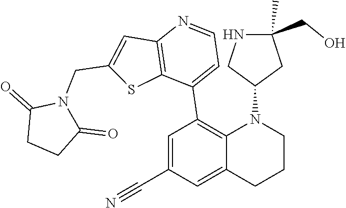 [SMILES: C[C@]1(CO)C[C@H](N2CCCc3cc(C#N)cc(-c4ccnc5cc(CN6C(=O)CCC6=O)sc45)c32)CN1] |
| 210 | 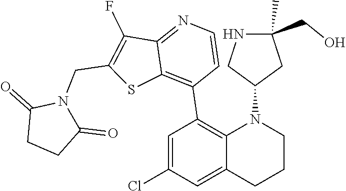 [SMILES: C[C@]1(CO)C[C@H](N2CCCc3cc(Cl)cc(-c4ccnc5c(F)c(CN6C(=O)CCC6=O)sc45)c32)CN1] |
| 211 | 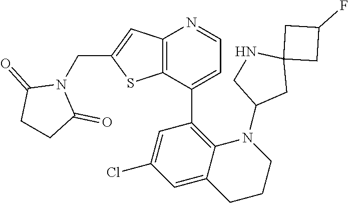 [SMILES: O=C1CCC(=O)N1Cc1cc2nccc(-c3cc(Cl)cc4c3N(C3CNC5(CC(F)C5)C3)CCC4)c2s1] |
| 212a | 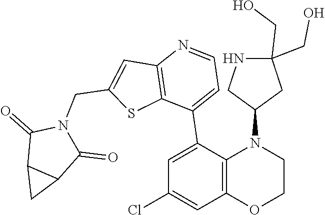 [SMILES: O=C1C2CC2C(=O)N1Cc1cc2nccc(-c3cc(Cl)cc4c3N([C@H]3CNC(CO)(CO)C3)CCO4)c2s1] |
| 212b | 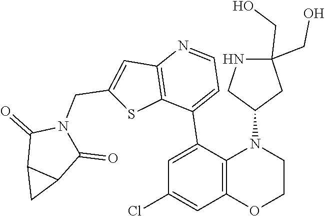 [SMILES: O=C1C2CC2C(=O)N1Cc1cc2nccc(-c3cc(Cl)cc4c3N([C@@H]3CNC(CO)(CO)C3)CCO4)c2s1] |
| 213a | 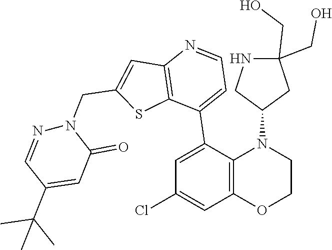 [SMILES: CC(C)(C)c1cnn(Cc2cc3nccc(-c4cc(Cl)cc5c4N([C@@H]4CNC(CO)(CO)C4)CCO5)c3s2)c(=O)c1] |
| 213b | 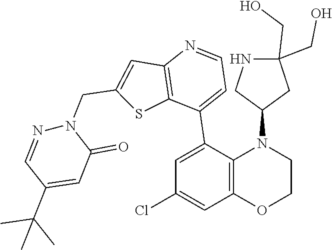 [SMILES: CC(C)(C)c1cnn(Cc2cc3nccc(-c4cc(Cl)cc5c4N([C@H]4CNC(CO)(CO)C4)CCO5)c3s2)c(=O)c1] |
| 214 | 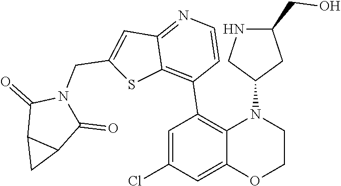 [SMILES: O=C1C2CC2C(=O)N1Cc1cc2nccc(-c3cc(Cl)cc4c3N([C@@H]3CN[C@@H](CO)C3)CCO4)c2s1] |
| 215 | 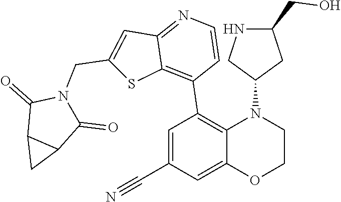 [SMILES: N#Cc1cc2c(c(-c3ccnc4cc(CN5C(=O)C6CC6C5=O)sc34)c1)N([C@@H]1CN[C@@H](CO)C1)CCO2] |
| 216 | 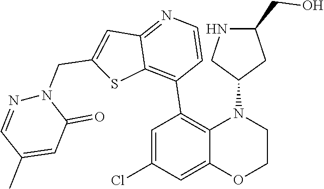 [SMILES: Cc1cnn(Cc2cc3nccc(-c4cc(Cl)cc5c4N([C@@H]4CN[C@@H](CO)C4)CCO5)c3s2)c(=O)c1] |
| 217 | 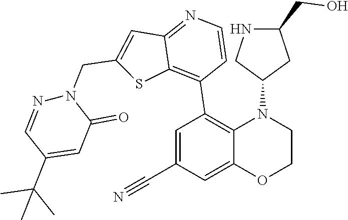 [SMILES: CC(C)(C)c1cnn(Cc2cc3nccc(-c4cc(C#N)cc5c4N([C@@H]4CN[C@@H](CO)C4)CCO5)c3s2)c(=O)c1] |
| 218 | 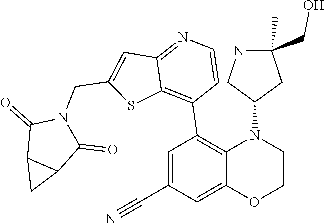 [SMILES: C[C@]1(CO)C[C@H](N2CCOc3cc(C#N)cc(-c4ccnc5cc(CN6C(=O)C7CC7C6=O)sc45)c32)CN1] |
| 219 | 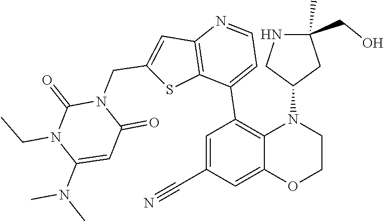 [SMILES: CCn1c(N(C)C)cc(=O)n(Cc2cc3nccc(-c4cc(C#N)cc5c4N([C@@H]4CN[C@@](C)(CO)C4)CCO5)c3s2)c1=O] |
| 220 | 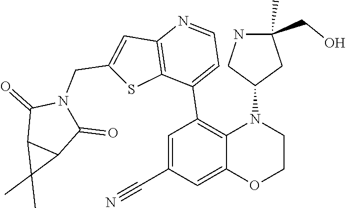 [SMILES: CC1(C)C2C(=O)N(Cc3cc4nccc(-c5cc(C#N)cc6c5N([C@@H]5CN[C@@](C)(CO)C5)CCO6)c4s3)C(=O)C21] |
| 221 | 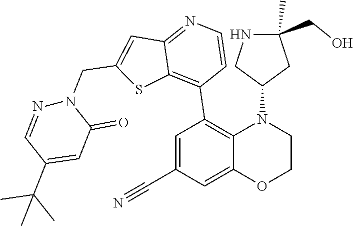 [SMILES: CC(C)(C)c1cnn(Cc2cc3nccc(-c4cc(C#N)cc5c4N([C@@H]4CN[C@@](C)(CO)C4)CCO5)c3s2)c(=O)c1] |
| 222 | 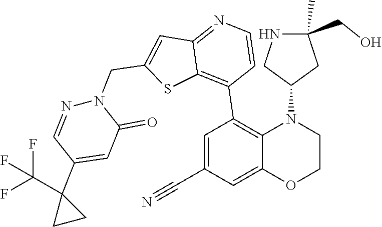 [SMILES: C[C@]1(CO)C[C@H](N2CCOc3cc(C#N)cc(-c4ccnc5cc(Cn6ncc(C7(C(F)(F)F)CC7)cc6=O)sc45)c32)CN1] |
| 223 | 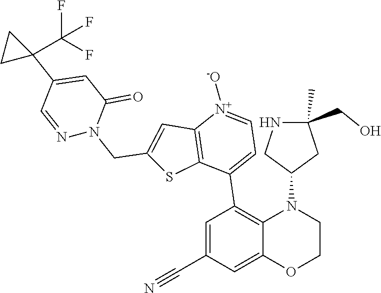 |
| 224 | 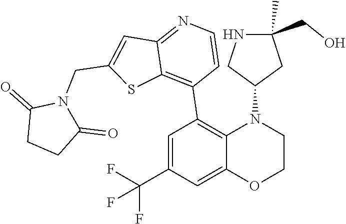 [SMILES: C[C@]1(CO)C[C@H](N2CCOc3cc(C(F)(F)F)cc(-c4ccnc5cc(CN6C(=O)CCC6=O)sc45)c32)CN1] |
| 225 | 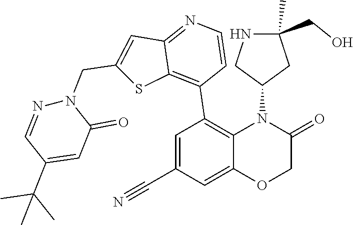 [SMILES: CC(C)(C)c1cnn(Cc2cc3nccc(-c4cc(C#N)cc5c4N([C@@H]4CN[C@@](C)(CO)C4)C(=O)CO5)c3s2)c(=O)c1] |
| 226 | 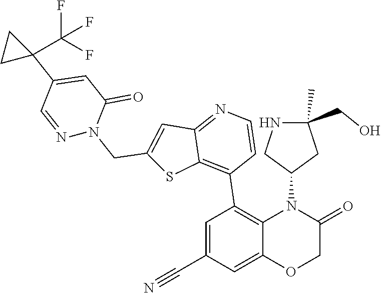 [SMILES: C[C@]1(CO)C[C@H](N2C(=O)COc3cc(C#N)cc(-c4ccnc5cc(Cn6ncc(C7(C(F)(F)F)CC7)cc6=O)sc45)c32)CN1] |
| 227a 227b 227c 227d | 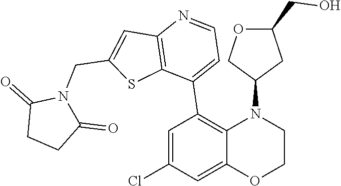 [SMILES: O=C1CCC(=O)N1Cc1cc2nccc(-c3cc(Cl)cc4c3N([C@H]3CO[C@@H](CO)C3)CCO4)c2s1] |
| | 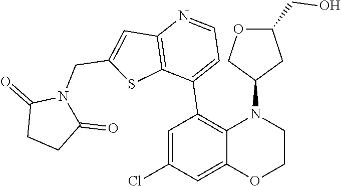 [SMILES: O=C1CCC(=O)N1Cc1cc2nccc(-c3cc(Cl)cc4c3N([C@H]3CO[C@H](CO)C3)CCO4)c2s1] |
| | 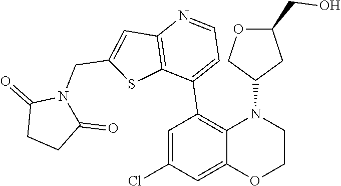 |
| | 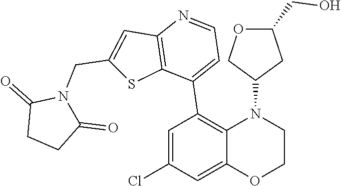 |
| 228 | 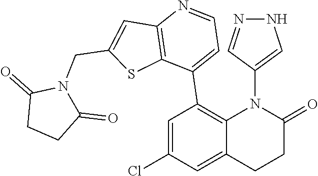 [SMILES: O=C1CCC(=O)N1Cc1cc2nccc(-c3cc(Cl)cc4c3N(c3cn[nH]c3)C(=O)CC4)c2s1] |
| 229a 229b | 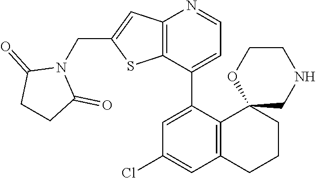 [SMILES: O=C1CCC(=O)N1Cc1cc2nccc(-c3cc(Cl)cc4c3[C@]3(CCC4)CNCCO3)c2s1] |
| | 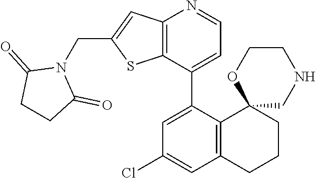 [SMILES: O=C1CCC(=O)N1Cc1cc2nccc(-c3cc(Cl)cc4c3[C@@]3(CCC4)CNCCO3)c2s1] |
| 230a | 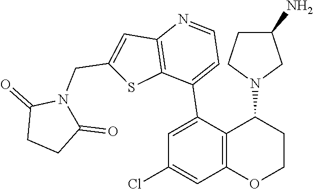 [SMILES: N[C@@H]1CCN([C@@H]2CCOc3cc(Cl)cc(-c4ccnc5cc(CN6C(=O)CCC6=O)sc45)c32)C1] |
| 230b | 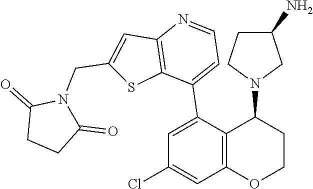 [SMILES: N[C@@H]1CCN([C@H]2CCOc3cc(Cl)cc(-c4ccnc5cc(CN6C(=O)CCC6=O)sc45)c32)C1] |

Further Forms of Compounds Disclosed Herein

Isomers Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, N-oxide, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising a compound described herein, or a pharmaceutically acceptable salt, solvate, N-oxide, or stereoisomer thereof, and a pharmaceutically acceptable excipient.

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. In some embodiments, the present method involve the administration of about 0.1 μg to about 50 mg of at least one compound described herein per kg body weight of the subject. For a 70 kg patient, dosages of from about 10 μg to about 200 mg of the compound disclosed herein would be more commonly used, depending on a subject's physiological response.

By way of example only, the dose of the compound described herein for methods of treating a disease as described herein is about 0.001 to about 1 mg/kg body weight of the subject per day, for example, about 0.001 mg, about 0.002 mg, about 0.005 mg, about 0.010 mg, 0.015 mg, about 0.020 mg, about 0.025 mg, about 0.050 mg, about 0.075 mg, about 0.1 mg, about 0.15 mg, about 0.2 mg, about 0.25 mg, about 0.5 mg, about 0.75 mg, or about 1 mg/kg body weight per day. In some embodiments, the dose of compound described herein for the described methods is about 1 to about 1000 mg/kg body weight of the subject being treated per day, for example, about 1 mg, about 2 mg, about 5 mg, about 10 mg, about 15 mg, about 20 mg, about 25 mg, about 50 mg, about 75 mg, about 100 mg, about 150 mg, about 200 mg, about 250 mg, about 500 mg, about 750 mg, or about 1000 mg per day.

Methods of Treatment

Disclosed herein is a method of treating a USP7-mediated disorder comprising administering to a subject in need thereof a therapeutically effective amount of a compound disclosed herein, or a pharmaceutically acceptable salt, solvate, N-oxide, or stereoisomer thereof. The compounds disclosed herein, or pharmaceutically acceptable salts, solvates, or stereoisomers thereof, are useful for the inhibition of USP7.

Disclosed herein is a method of treating cancer comprising administering to a subject in need thereof a therapeutically effective amount of a compound disclosed herein, or a pharmaceutically acceptable salt, solvate, N-oxide, or stereoisomer thereof.

In some embodiments, the cancer is a solid tumor. In some embodiments, the solid tumor is ovarian cancer, breast cancer, lung cancer, pancreatic cancer, kidney cancer, melanoma, liver cancer, colon cancer, sarcoma, brain cancer, or prostate cancer.

In some embodiments, the cancer is a blood cancer. In some embodiments, the blood cancer is leukemia, lymphoma, or multiple myeloma. In some embodiments, the leukemia is acute myeloid leukemia (AML). In some embodiments, the blood cancer is a myeloproliferative neoplasm (MPN). In some embodiments, the MPN is chronic myeloid leukemia (CML), chronic neutrophilic leukemia (CNL), polycythemia vera (PV), primary myelofibrosis (PMF), essential thrombocythemia (ET), or chronic eosinophilic leukemia.

In some embodiments, the cancer is liposarcoma, neuroblastoma, glioblastoma, breast cancer, bladder cancer, glioma, adrenocortical cancer, multiple myeloma, colorectal cancer, colon cancer, prostate cancer, non-small cell lung cancer, Human Papilloma Virus-associated cervical cancer, oropharyngeal cancer, penis cancer, ovarian cancer, anal cancer, thyroid cancer, vaginal cancer, Epstein-Barr Virus-associated nasopharyngeal carcinoma, gastric cancer, rectal cancer, thyroid cancer, Hodgkin lymphoma, diffuse large B-cell lymphoma, Ewing sarcoma,

Disclosed herein is a method of treating a neurodegenerative disease comprising administering to a subject in need thereof a therapeutically effective amount of a compound disclosed herein, or a pharmaceutically acceptable salt, solvate, N-oxide, or stereoisomer thereof. In some embodiments, the neurodegenerative disease is Alzheimer's disease, multiple sclerosis, Huntington's disease, infectious meningitis, encephalomyelitis, Parkinson's disease, amyotrophic lateral sclerosis, or encephalitis.

Disclosed herein is a method of treating a viral infection comprising administering to a subject in need thereof a therapeutically effective amount of a compound disclosed herein, or a pharmaceutically acceptable salt, solvate, N-oxide, or stereoisomer thereof. In some embodiments, the viral infection is herpes simplex-1 or -2 viral infection, hepatitis A, hepatitis C, SARS coronavirus infection, Epstein-Barr virus, rhinoviral infection, adenoviral infection, or poliomyelitis.

Disclosed herein is a method of treating an inflammatory disease comprising administering to a subject in need thereof a therapeutically effective amount of a compound disclosed herein, or a pharmaceutically acceptable salt, solvate, N-oxide, or stereoisomer thereof. In some embodiments, the inflammatory disease is inflammatory bowel diseases including, but not limited to, ileitis, ulcerative colitis, Barrett's syndrome, or Crohn's disease.

Disclosed herein is a method of inducing cell cycle arrest, apoptosis in tumor cells, and/or enhanced tumor-specific T cell immunity comprising administering to a subject in need thereof a therapeutically effective amount of a compound disclosed herein, or a pharmaceutically acceptable salt, solvate, N-oxide, or stereoisomer thereof.

Disclosed herein is a method of treating a patient in need thereof comprising administering to a subject in need thereof a therapeutically effective amount of a compound disclosed herein, or a pharmaceutically acceptable salt, solvate, N-oxide, or stereoisomer thereof, wherein the patient in need thereof is selected for treatment based on gene amplification and/or elevated tumor expression of USP7, MDM2 or MDM4 relative to tissue-matched expression.

Disclosed herein is a method of treating a patient in need thereof comprising administering to a subject in need thereof a therapeutically effective amount of a compound disclosed herein, or a pharmaceutically acceptable salt, solvate, N-oxide, or stereoisomer thereof, wherein the patient in need thereof is selected for treatment based on tumor expression of wild type TP53 or based on the tumor immune cell composition, specifically elevated regulatory T lymphocytes, CD4+CD25+FoxP3+ T cells.

Combination Therapy

In certain instances, the compound described herein, or a pharmaceutically acceptable salt, solvate, N-oxide, or stereoisomer thereof, is administered in combination with a second therapeutic agent.

In some embodiments, the benefit experienced by a patient is increased by administering one of the compounds described herein with a second therapeutic agent (which also includes a therapeutic regimen) that also has therapeutic benefit.

In one specific embodiment, a compound described herein, or a pharmaceutically acceptable salt, solvate, N-oxide, or stereoisomer thereof, is co-administered with a second therapeutic agent, wherein the compound described herein, or a pharmaceutically acceptable salt, solvate, N-oxide, or stereoisomer thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply additive of the two therapeutic agents or the patient experiences a synergistic benefit.

In certain embodiments, different therapeutically-effective dosages of the compounds disclosed herein will be utilized in formulating a pharmaceutical composition and/or in treatment regimens when the compounds disclosed herein are administered in combination with a second therapeutic agent. Therapeutically-effective dosages of drugs and other agents for use in combination treatment regimens are optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt, solvate, N-oxide, or stereoisomer thereof, is initiated prior to, during, or after treatment with a second agent described herein, and continues until any time during treatment with the second agent or after termination of treatment with the second agent. It also includes treatments in which a compound described herein, or a pharmaceutically acceptable salt, solvate, N-oxide, or stereoisomer thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated, and so forth. In additional embodiments, when co-administered with a second therapeutic agent, the compound provided herein is administered either simultaneously with the second therapeutic agent, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt, solvate, N-oxide, or stereoisomer thereof, as well as combination therapies, are administered before, during, or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, the compound of described herein, or a pharmaceutically acceptable salt, solvate, N-oxide, or stereoisomer thereof, is administered in combination with an adjuvant. In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced).

In some embodiments, the compound disclosed herein, or a pharmaceutically acceptable salt thereof, is administered in combination with an anti-cancer agent.

In some embodiments, the anti-cancer agent is a hormone blocking therapy. Hormone blocking therapy includes the use of agents that block the production of estrogens or block the estrogen receptors. In some embodiments, hormone blocking therapy includes the use of estrogen receptor modulators and/aromatase inhibitors. Estrogen receptor modulators include triphenylethylene derivatives (e.g., tamoxifen, toremifene, droloxifene, 3-hydroxytamoxifen, idoxifene, TAT-59 (a phosphorylated derivative of 4-hydroxytamoxifen) and GW5638 (a carboxylic acid derivative of tamoxifen)); non-steroidal estrogen receptor modulators (e.g., raloxifene, LY353381 (SERM3) and LY357489); steroidal estrogen receptor modulators (e.g., ICI-182,780). Aromatase inhibitors include steroidal aromatase inhibitors and non-steroidal aromatase inhibitors. Steroidal aromatase inhibitors include, but are not limited to, exemestane. Non-steroidal aromatase inhibitors include, but are not limited to, anastrozole and letrozole.

In certain embodiments, compounds disclosed herein are used in combination with one or more passive immunotherapies, including but not limited to, naked monoclonal antibody drugs and conjugated monoclonal antibody drugs. Examples of naked monoclonal antibody drugs that can be used include, but are not limited to, rituximab, an antibody against the CD20 antigen; trastuzumab, an antibody against the HER2 protein; alemtuzumab, an antibody against the CD52 antigen; cetuximab, an antibody against the EGFR protein; and bevacizumab which is an anti-angiogenesis inhibitor of VEGF protein.

Examples of conjugated monoclonal antibodies include, but are not limited to, radiolabeled antibody ibritumomab tiuxetan; radiolabeled antibody tositumomab; and immunotoxin gemtuzumab ozogamicin which contains calicheamicin; BL22, an anti-CD22 monoclonal antibody-immunotoxin conjugate; radiolabeled antibodies such as OncoScint (Registered trademark) and ProstaScint (Registered trademark); brentuximab vedotin; and ado-trastuzumab emtansine.

Further examples of therapeutic antibodies that can be used include, but are not limited to, abciximab, an antibody against the glycoprotein IIb/IIIa receptor on platelets; daclizumab, an immunosuppressive, humanized anti-CD25 monoclonal antibody; edrecolomab, a murine anti-17-IA cell surface antigen IgG2a antibody; BEC2, a murine anti-idiotype (GD3 epitope) IgG antibody; IMC-C₂₂₅, a chimeric anti-EGFR IgG antibody; VITAXIN (Registered Trademark) a humanized anti-aVbeta 3 integrin antibody; Campath 1H/LDP-03, a humanized anti CD52 IgG1 antibody; Smart M195, a humanized anti-CD33 IgG antibody; epratuzumab, a humanized anti-CD22 IgG antibody; Lymphoscan; visilizumab; CM3, a humanized anti-ICAM3 antibody; IDEC-114 a primatized anti-CD80 antibody; IDEC-131 a humanized anti-CD40L antibody; IDEC-151 a primatized anti-CD4 antibody; IDEC-152 a primatized anti-CD23 antibody; SMART anti-CD3, a humanized anti-CD3 IgG; 5G1.1, a humanized anti-complement factor 5 (C₅) antibody; D2E7, a humanized anti-TNF-alpha antibody; CDP870, a humanized anti-TNF-alpha Fab fragment; IDEC-151, a primatized anti-CD4 IgG1 antibody; MDX-CD4, a human anti-CD4 IgG antibody; CD20-streptdavidin (+biotin-yttrium 90); CDP571, a humanized anti-TNF-alpha IgG4 antibody; LDP-02, a humanized anti-alpha 4beta 7 antibody; OrthoClone OKT4A, a humanized anti-CD4 IgG antibody; ANTOVA (Registered Trademark), a humanized anti-CD40L IgG antibody; ANTEGREN (Registered Trademark), a humanized anti-VLA-4 IgG antibody; and CAT-152, a human anti-TGF-beta 2 antibody.

In some embodiments, the second therapeutic agent for use in combination with a compound disclosed herein, or a pharmaceutically acceptable salt thereof, include one or more of the following: abiraterone; abarelix; adriamycin; actinomycin; acivicin; aclarubicin; acodazole hydrochloride; acronine; adozelesin; aldesleukin; alemtuzumab; allopurinol; alitretinoin; altretamine; ambomycin; ametantrone acetate; aminoglutethimide; aminolevulinic acid; amifostine; amsacrine; anastrozole; anthramycin; aprepitant; arsenic trioxide; asparaginase; asperlin; azacitidine; azetepa; azotomycin; batimastat; bendamustine hydrochloride; benzodepa; bevacizumab; bexarotene; bicalutamide; bisantrene hydrochloride; bisnafide dimesylate; bizelesin; bleomycin; bleomycin sulfate; bortezomib; brequinar sodium; bropirimine; busulfan; cactinomycin; calusterone; caracemide; carbetimer; carboplatin; carmustine; carubicin hydrochloride; carzelesin; capecitabine; cedefingol; cetuximab; chlorambucil; cirolemycin; cisplatin; cladribine; clofarabine; crisnatol mesylate; cyclophosphamide; cytarabine; dacarbazine; dasatinib; daunorubicin hydrochloride; dactinomycin; darbepoetin alfa; decitabine; degarelix; denileukin diftitox; dexormaplatin; dexrazoxane hydrochloride; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; eltrombopag olamine; enloplatin; enpromate; epipropidine; epirubicin hydrochloride; epoetin alfa; erbulozole; erlotinib hydrochloride; esorubicin hydrochloride; estramustine; estramustine phosphate sodium; etanidazole; etoposide; etoposide phosphate; etoprine; everolimus; exemestane; fadrozole hydrochloride; fazarabine; fenretinide; filgrastim; floxuridine; fludarabine phosphate; fluorouracil; fluorocitabine; fosquidone; fostriecin sodium; fulvestrant; gefitinib; gemcitabine; gemcitabine hydrochloride; gemcitabine cisplatin; gemtuzumab ozogamicin; goserelin acetate; histrelin acetate; hydroxyurea; idarubicin hydrochloride; ifosfamide; limofosine; ibritumomab tiuxetan; idarubicin; ifosfamide; imatinib mesylate; imiquimod; interleukin Il (including recombinant interleukin II, or rlL2), interferon alfa-2a; interferon alfa-2b; interferon alfa-n1; interferon alfa-n3; interferon beta-1a; interferon gamma-lb; iproplatin; irinotecan hydrochloride; ixabepilone; lanreotide acetate; lapatinib; lenalidomide; letrozole; leuprolide acetate; leucovorin calcium; leuprolide acetate; levamisole; liposomal cytarabine; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; methoxsalen; metoprine; meturedepa; mitindomide; mitocarcin; mitocromin; mitogillin; mitomalcin; mitomycin C; mitosper; mitotane; mitoxantrone hydrochloride; mycophenolic acid; nandrolone phenpropionate; nelarabine; nilotinib; nocodazole; nofetumomab; nogalamycin; ofatumumab; oprelvekin; ormaplatin; oxaliplatin; oxisuran; paclitaxel; palifermin; palonosetron hydrochloride; pamidronate; pegfilgrastim; pemetrexed disodium; pentostatin; panitumumab; pazopanib hydrochloride; pemetrexed disodium; plerixafor; pralatrexate; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; quinacrine; raloxifene hydrochloride; rasburicase; recombinant HPV bivalent vaccine; recombinant HPV quadrivalent vaccine; riboprine; rogletimide; rituximab; romidepsin; romiplostim; safingol; safingol hydrochloride; sargramostim; semustine; simtrazene; sipuleucel-T; sorafenib; sparfosate sodium; sparsomycin; spirogermanium hydrochloride; spiromustine; spiroplatin; streptonigrin; streptozocin; sulofenur; sunitinib malate; talisomycin; tamoxifen citrate; tecogalan sodium; tegafur; teloxantrone hydrochloride; temozolomide; temoporfin; temsirolimus; teniposide; teroxirone; testolactone; thalidomide; thiamiprine; thioguanine; thiotepa; tiazofurin; tirapazamine; topotecan hydrochloride; toremifene; tositumomab and I 131 Iodine tositumomab; trastuzumab; trestolone acetate; tretinoin; triciribine phosphate; trimetrexate; trimetrexate glucuronate; triptorelin; tubulozole hydrochloride; uracil mustard; uredepa; valrubicin; vapreotide; verteporfin; vinblastine; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorinostat; vorozole; zeniplatin; zinostatin; zoledronic acid; and zorubicin hydrochloride.

In some embodiments, the second therapeutic agent is an alkylating agent. Examples of alkylating agents for use in combination with a compound disclosed herein, or a pharmaceutically acceptable salt thereof, include, but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomustine, semustine, streptozocin, etc.), or triazenes (decarbazine, etc.).

Other agents that are optionally used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

In some embodiments, the second therapeutic agent is an immunotherapy agent. Examples of immunotherapy agents for use in combination with a compound disclosed herein, or a pharmaceutically acceptable salt thereof, include, but are not limited to, checkpoint inhibitors (e.g., anti-PD1 and anti-PD-L1 inhibitors), cancer vaccines (e.g., sipuleucel-T), oncolytic viruses (e.g., talimogene laherparepvec), cytokines (e.g., IL-2 and INF-alpha), CAR-T cells.

In some embodiments, the second therapeutic agent is an immune checkpoint inhibitor.

In some embodiments, the immune checkpoint inhibitor is selected from the group consisting of PD-1 inhibitors, PD-L1 inhibitors, PD-L2 inhibitors, CTLA-4 inhibitors, OX40 agonists, and 4-1BB agonists.

In some embodiments, the checkpoint inhibitors is a programmed cell death protein 1 (PD-1) inhibitor or a programmed cell death ligand 1 (PD-L1) inhibitor. In some embodiments, the PD-1 inhibitor or the PD-L1 inhibitor is an antibody or antigen-binding fragment against PD-1 or PD-L1.

In some embodiments, the PD-1 inhibitor is selected from pembrolizumab, nivolumab, cemiplimab, lambrolizumab, AMP-224, sintilimab, toripalimab, camrelizumab, tislelizumab, dostarlimab (GSK), PDR001 (Novartis), MGA012 (Macrogenics/Incyte), GLS-010 (Arcus/Wuxi), AGEN2024 (Agenus), cetrelimab (Janssen), ABBV-181 (Abbvie), AMG-404 (Amgen). BI-754091 (Boehringer Ingelheim), CC-90006 (Celgene), JTX-4014 (Jounce), PF-06801591 (Pfizer), and genolimzumab (Apollomics/Genor BioPharma). In some embodiments, the PD-1 inhibitor is pembrolizumab. In some embodiments, the PD-1 inhibitor is nivolumab. In some embodiments, the PD-1 inhibitor is cemiplimab. In some embodiments, the PD-1 inhibitor is lambrolizumab. In some embodiments, the PD-1 inhibitor is AMP-224. In some embodiments, the PD-1 inhibitor is sintilimab. In some embodiments, the PD-1 inhibitor is toripalimab. In some embodiments, the PD-1 inhibitor is camrelizumab. In some embodiments, the PD-1 inhibitor is tislelizumab.

In some embodiments, the PD-L1 inhibitor is selected from atezolizumab, avelumab, and durvalumab, ASC22 (Alphamab/Ascletis), CX-072 (Cytomx), CS1001 (Cstone), cosibelimab (Checkpoint Therapeutics), INCB86550 (Incyte), and TG-1501 (TG Therapeutics). In some embodiments, the PD-L1 inhibitor is atezolizumab. In some embodiments, the PD-L1 inhibitor is avelumab. In some embodiments, the PD-L1 inhibitor is durvalumab.

In some embodiments, the immune checkpoint inhibitor is a cytotoxic T-lymphocyte protein 4 (CTLA4) inhibitor. In some embodiments, the CTLA4 inhibitor is an antibody or antigen-binding fragment against CTLA4. In some embodiments, the CTLA4 inhibitor is ipilimumab or tremelimumab.

In some embodiments, the immune checkpoint inhibitor is a CTLA-4 inhibitor. In some embodiments, the CTLA-4 inhibitor is selected from tremelimumab, ipilimumab, and AGEN-1884 (Agenus). In some embodiments, the CTLA-4 inhibitor is tremelimumab. In some embodiments, the e CTLA-4 inhibitor is ipilimumab. In some embodiments, the checkpoint inhibitors is a programmed cell death ligand 2 (PD-L2) inhibitor. In some embodiments, the immune checkpoint inhibitor is a OX40 agonist. In some embodiments, the immune checkpoint inhibitor is a 4-1BB agonist.

In some embodiments, a compound disclosed herein, or a pharmaceutically acceptable salt thereof, is used in combination with anti-emetic agents to treat nausea or emesis, which results from the use of a compound disclosed herein, or a pharmaceutically acceptable salt thereof, anti-cancer agent(s) and/or radiation therapy. Anti-emetic agents include, but are not limited to: neurokinin-1 receptor antagonists, 5HT3 receptor antagonists (such as ondansetron, granisetron, tropisetron, palonosetron, and zatisetron), GABAB receptor agonists (such as baclofen), corticosteroids (such as dexamethasone, prednisone, prednisolone, or others), dopamine antagonists (such as, but not limited to, domperidone, droperidol, haloperidol, chlorpromazine, promethazine, prochlorperazine, metoclopramide), antihistamines (H1 histamine receptor antagonists, such as but not limited to, cyclizine, diphenhydramine, dimenhydrinate, meclizine, promethazine, hydroxyzine), cannabinoids (such as but not limited to, cannabis, marinol, dronabinol), and others (such as, but not limited to, trimethobenzamide; ginger, emetrol, propofol).

In some embodiments, a compound disclosed herein, or a pharmaceutically acceptable salt thereof, is used in combination with an agent useful in the treatment of anemia. Such an anemia treatment agent is, for example, a continuous eythropoiesis receptor activator (such as epoetin-α).

In some embodiments, a compound disclosed herein, or a pharmaceutically acceptable salt thereof, is used in combination with an agent useful in the treatment of neutropenia. Examples of agents useful in the treatment of neutropenia include, but are not limited to, a hematopoietic growth factor which regulates the production and function of neutrophils such as a human granulocyte colony stimulating factor, (G-CSF). Examples of a G-CSF include filgrastim.

In one embodiment, a compound disclosed herein, or a pharmaceutically acceptable salt thereof, is administered to a mammal in combination with a non-steroidal anti-inflammatory drug (NSAID). NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, COX-2 specific inhibitors (such as, but not limited to, celecoxib, rofecoxib, valdecoxib, parecoxib, etoricoxib, lumiracoxib, CS-502, JTE-522, L-745 337, and NS398).

In some embodiments, a compound disclosed herein, or a pharmaceutically acceptable salt thereof, is used in combination with radiation therapy (or radiotherapy). Radiation therapy is the treatment of cancer and other diseases with ionizing radiation. Radiation therapy is optionally used to treat localized solid tumors, such as cancers of the skin, tongue, larynx, brain, breast, prostate, colon, uterus, and/or cervix. It is also optionally used to treat leukemia and lymphoma (cancers of the blood-forming cells and lymphatic system, respectively).

In some embodiments, a compound disclosed herein, or a pharmaceutically acceptable salt thereof, is used in combination with a histone deacetylase (HDAC) inhibitor, a proteasome inhibitor, a BET inhibitor, a B-cell lymphoma 2 (Bcl-2) inhibitor, or any combination thereof.

### EXAMPLES

### Example 1: 1-((7-(1-(azetidin-3-yl)-6-chloro-1,2,3,4-tetrahydroquinolin-8-yl)thieno[3,2-b]pyridin-2-yl)methyl)pyrrolidine-2,5-dione, formic acid salt

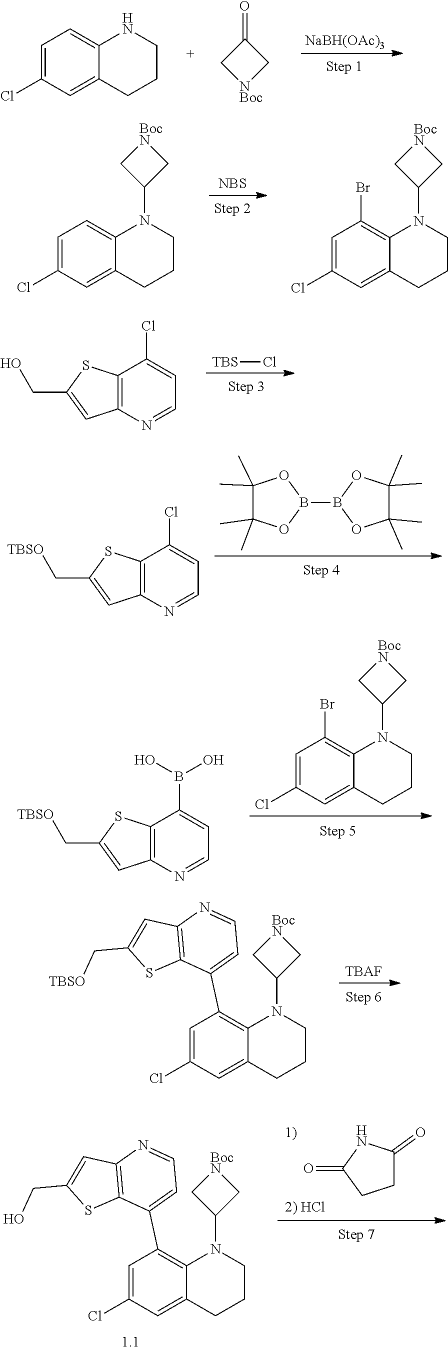

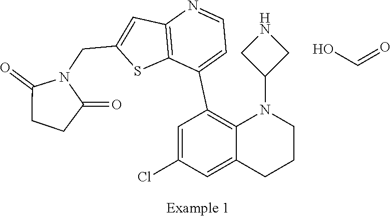

### Step 1: tert-butyl 3-(6-chloro-3,4-dihydroquinolin-1(2H)-yl)azetidine-1-carboxylate

To a solution of 6-chloro-1,2,3,4-tetrahydroquinoline (1.5 g, 8.65 mmol) and tert-butyl 3-oxoazetidine-1-carboxylate (2.2 g, 12.97 mmol) in 1,2-dichloroethane (20 mL) and acetic acid (1.0 g) was added sodium triacetoxyborohydride (3.7 g, 17.30 mmol) at 25° C. After stirring at 25° C. for 1 h, the mixture was quenched by addition of saturated aqueous sodium bicarbonate (40 mL) and extracted with ethyl acetate (30 mL×3). The combined organic layers were washed with brine (60 mL), dried over anhydrous sodium sulfate, filtered and concentrated under reduced pressure to afford the title compound, which was used in next step without further purification. LCMS R_{T}=1.433 min, m/z=323.2 [M+H]⁺.

### Step 2: tert-butyl 3-(8-bromo-6-chloro-3,4-dihydroquinolin-1(2H)-yl)azetidine-1-carboxylate

To a solution of tert-butyl 3-(6-chloro-3,4-dihydro-2H-quinolin-1-yl)azetidine-1-carboxylate (2.5 g, 7.74 mmol) in N,N-dimethylformamide (15 mL) was added N-bromosuccinimide (1.4 g, 7.74 mmol) at 25° C. After stirring for 4 min at 25° C., the reaction was quenched by addition of saturated aqueous sodium bicarbonate (40 mL) and extracted with ethyl acetate (30 mL×3). The combined organic layers were washed with brine (60 mL), dried over anhydrous sodium sulfate, filtered and concentrated under reduced pressure. The residue was purified by column chromatography to afford the title compound. LCMS R_{T}=1.521 min, m/z=403.2 [M+H]⁺.

### Step 3: 2-(((tert-butyldimethylsilyl)oxy)methyl)-7-chlorothieno[3,2-b]pyridine

To a solution of (7-chlorothieno[3,2-b]pyridin-2-yl)methanol (20.0 g, 100.17 mmol) and imidazole (27.3 g, 400.69 mmol) in dichloromethane (300 mL) was added tert-butyldimethylsilyl chloride (37.8 g, 250.43 mmol). After stirring at 25° C. for 12 h under nitrogen atmosphere, the reaction mixture was diluted with water (100 mL) and extracted with dichloromethane (200 mL×2). The combined organic layers were washed with brine (20 mL), dried over anhydrous sodium sulfate, filtered and concentrated under reduced pressure. The residue was purified by column chromatography to afford the title compound. LCMS R_{T}=1.615 m/z=314.1 [M+H]⁺.

### Step 4: (2-(((tert-butyldimethylsilyl)oxy)methyl)thieno[3,2-b]pyridin-7-yl)boronic acid

To a solution of 2-(((tert-butyldimethylsilyl)oxy)methyl)-7-chlorothieno[3,2-b]pyridine (160 mg, 0.51 mmol), bis(pinacolato)diboron (389 mg, 1.53 mmol) and potassium acetate (150 mg, 1.53 mmol) in dioxane (3 mL) was added dichloro[1,1′-bis(diphenylphosphino)ferrocene]palladium(II) (75 mg, 0.10 mmol) under nitrogen atmosphere. After stirring at 140° C. for 1 h in a microwave oven, the reaction was filtered and the filtrate was concentrated under reduced pressure afford the title compound, which was used in next step without further purification. LCMS R_{T}=0.873 m/z=324.0 [M+H]⁺.

### Step 5: tert-butyl 3-(8-(2-(((tert-butyldimethylsilyl)oxy)methyl)thieno[3,2-b]pyridin-7-yl)-6-chloro-3,4-dihydroquinolin-1(2H)-yl)azetidine-1-carboxylate

A mixture of tert-butyl 3-(8-bromo-6-chloro-3,4-dihydro-2H-quinolin-1-yl)azetidine-1-carboxylate (600 mg, 1.49 mmol), [2-[[tert-butyl(dimethyl)silyl]oxymethyl]thieno[3,2-b]pyridin-7-yl]boronic acid (482 mg, 1.49 mmol), dichloro[1,1′-bis(diphenylphosphino)ferrocene]palladium(II) (218 mg, 0.3 mmol) and cesium carbonate (973 mg, 2.99 mmol) in water (2 mL) and dioxane (20 mL) was stirred at 110° C. for 2 h. The reaction mixture was diluted with water (30 mL) and extracted with ethyl acetate (30 mL×2). The combined organic layers were washed with brine (30 mL), dried over anhydrous sodium sulfate, filtered and concentrated under reduced pressure. The residue was purified by column chromatography to afford the title compound. LCMS R_{T}=1.148 min, m/z=600.2 [M+H]⁺.

### Step 6: tert-butyl 3-(6-chloro-8-(2-(hydroxymethyl)thieno[3,2-b]pyridin-7-yl)-3,4-dihydroquinolin-1(2H)-yl)azetidine-1-carboxylate

To a solution of tert-butyl 3-[8-[2-[[tert-butyl(dimethyl)silyl]oxymethyl]thieno[3,2-b]pyridin-7-yl]-6-chloro-3,4-dihydro-2H-quinolin-1-yl]azetidine-1-carboxylate (900 mg, 1.50 mmol) in tetrahydrofuran (15 mL) was added tetrabutylammonium fluoride (1.5 mL, 1.0 M in tetrahydrofuran). After stirring for 30 min at 25° C., the reaction mixture was concentrated under reduced pressure. The residue was purified by column chromatography to afford the title compound. LCMS R_{T}=0.892 min, m/z=486.2 [M+H]⁺.

### Step 7

To a solution of tert-butyl 3-[6-chloro-8-[2-(hydroxymethyl)thieno[3,2-b]pyridin-7-yl]-3,4-dihydro-2H-quinolin-1-yl]azetidine-1-carboxylate (150 mg, 0.31 mmol), succinimide (61 mg, 0.62 mmol) and triphenylphosphine (162 mg, 0.62 mmol) in tetrahydrofuran (2 mL) was added diisopropyl azodicarboxylate (125 mg, 0.62 mmol) at 0° C. After stirring at 0° C. for 10 min, the reaction was concentrated under reduced pressure. The residue was dissolved in dioxane (5 mL) and hydrochloric acid (1.80 mL, 4 M in dioxane) was added. After stirring at 25° C. for 30 min, the reaction mixture was concentrated under reduced pressure. The residue was purified by RP-HPLC to afford Example 1. ¹H NMR (400 MHz, CD₃OD) δ=8.75 (d, J=5.2 Hz, 1H), 8.53 (s, 1H), 7.60-7.38 (m, 2H), 7.21 (s, 2H), 4.97 (s, 2H), 4.07-3.98 (m, 1H), 3.50 (br s, 2H), 3.47-3.22 (m, 4H) 2.93 (t, J=6.4 Hz, 2H), 2.77 (s, 4H), 1.93 (br. s, 2H). LCMS R_{T}=1.550 min, m/z=467.2 [M+H]⁺.

### Example 2: 1-((7-(6-chloro-1-(1-methylazetidin-3-yl)-1,2,3,4-tetrahydroquinolin-8-yl)thieno[3,2-b]pyridin-2-yl)methyl)pyrrolidine-2,5-dione, formic acid salt

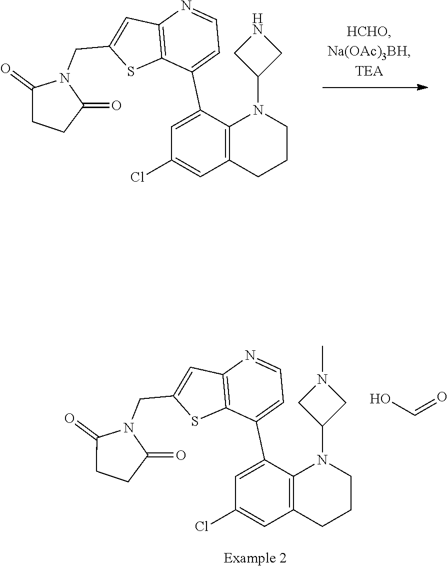

To a solution of 1-[[7-[1-(azetidin-3-yl)-6-chloro-3,4-dihydro-2H-quinolin-8-yl]thieno[3,2-b]pyridin-2-yl]methyl]pyrrolidine-2,5-dione (Example 1, Step 7, 30 mg, 0.064 mmol) in acetonitrile (0.5 mL) was added formaldehyde (10 mg, 0.13 mmol), sodium triacetoxyborohydride (41 mg, 0.19 mmol) and triethylamine (7 mg, 0.064 mol) at 25° C. After stirring at 25° C. for 1 h, the reaction was concentrated under reduced pressure. The residue was dissolved in hydrochloric acid (4 M in dioxane, 1.80 mL). After stirring at 25° C. for 30 min, the reaction was concentrated under reduced pressure. The residue was purified by RP-HPLC to afford Example 2. ¹H NMR (400 MHz, CD₃OD) δ=8.73 (d, J=4.8 Hz, 1H), 8.42 (br s, 1H), 7.38-7.59 (m, 2H), 7.04-7.27 (m, 2H), 4.95 (s, 4H), 3.87 (d, J=7.2 Hz, 1H), 3.48 (s, 4H), 2.91 (t, J=6.4 Hz, 2H), 2.77 (s, 4H), 2.60 (s, 3H), 1.78-1.98 (m, 2H). LCMS R_{T}=1.460 min, m/z=481.1 [M+H]⁺.

### Example 3: 1-((7-(6-chloro-1-(1-cyclobutylazetidin-3-yl)-1,2,3,4-tetrahydroquinolin-8-yl)thieno[3,2-b]pyridin-2-yl)methyl)pyrrolidine-2,5-dione, formic acid salt

Prepared from 1-[[7-[1-(azetidin-3-yl)-6-chloro-3,4-dihydro-2H-quinolin-8-yl]thieno[3,2-b]pyridin-2-yl]methyl]pyrrolidine-2,5-dione (Example 1, Step 7) and cyclobutanone, following the procedure described in the synthesis of Example 2. The final compound was purified by RP-HPLC. ¹H NMR (400 MHz, CD₃OD) δ=8.71 (d, J=4.8 Hz, 1H), 8.44 (s, 1H), 7.38-7.50 (m, 2H), 7.10-7.21 (m, 2H), 4.94 (s, 4H), 3.83 (t, J=8.0 Hz, 2H), 3.36-3.56 (m, 4H), 2.88 (t, J=6.4 Hz, 2H), 2.76 (s, 4H), 2.02 (d, J=7.6 Hz, 2H), 1.62-1.93 (m, 6H). LCMS R_{T}=1.030 min, m/z=521.2 [M+H]⁺.

### Example 4: 1-((7-(6-chloro-1-(1-ethylazetidin-3-yl)-1,2,3,4-tetrahydroquinolin-8-yl)thieno[3,2-b]pyridin-2-yl)methyl)pyrrolidine-2,5-dione, formic acid salt

Prepared from 1-[[7-[1-(azetidin-3-yl)-6-chloro-3,4-dihydro-2H-quinolin-8-yl]thieno[3,2-b]pyridin-2-yl]methyl]pyrrolidine-2,5-dione (Example 1, Step 7) and acetaldehyde following the procedure described in the synthesis of Example 2. The final compound was purified by RP-HPLC. ¹H NMR (400 MHz, CD₃OD) δ=8.72 (d, J=4.8 Hz, 1H), 8.43 (s, 1H), 7.37-7.57 (m, 2H), 7.04-7.28 (m, 2H), 4.95 (s, 4H), 3.87 (quin, J=8.4 Hz, 1H), 3.34-3.80 (m, 4H), 2.83-3.04 (m, 4H), 2.76 (s, 4H), 1.88 (s, 2H), 0.97 (t, J=7.2 Hz, 3H). LCMS R_{T}=1.023 min, m/z=495.1 [M+H]⁺.

### Example 5: 1-((7-(6-chloro-1-(1-cyclopropylazetidin-3-yl)-1,2,3,4-tetrahydroquinolin-8-yl)thieno[3,2-b]pyridin-2-yl)methyl)pyrrolidine-2,5-dione

To a solution of 1-[[7-[1-(azetidin-3-yl)-6-chloro-3,4-dihydro-2H-quinolin-8-yl]thieno[3,2-b]pyridin-2-yl]methyl]pyrrolidine-2,5-dione (Example 1, Step 7, 100 mg, 0.21 mmol) in methanol (0.5 mL) and ethanol (0.5 mL) was added (1-ethoxycyclopropoxy)-trimethyl-silane (224 mg, 1.28 mmol), acetic acid (129 mg, 2.14 mmol) and sodium cyanoborohydride (67 mg, 1 mmol) at 25° C. After stirring at 75° C. for 1 h, the reaction was concentrated under reduced pressure. The residue was purified by RP-HPLC to afford Example 5. ¹H NMR (400 MHz, CD₃OD) δ=8.69 (d, J=4.8 Hz, 1H), 7.35-7.54 (m, 2H), 7.14 (br d, J=1.6 Hz, 2H), 4.93 (s, 4H), 3.66-3.74 (m, 1H), 3.33-3.65 (m, 4H), 2.88 (t, J=6.4 Hz, 2H), 2.76 (s, 4H), 2.32-2.25 (m, 1H), 1.87 (s, 2H), 0.43-0.57 (m, 2H), 0.37 (d, J=2.0 Hz, 2H). LCMS R_{T}=1.248 min, m/z=507.2 [M+H]⁺.

### Example 6: 1-((7-(6-chloro-1-(1-(oxetan-3-yl)azetidin-3-yl)-1,2,3,4-tetrahydroquinolin-8-yl)thieno[3,2-b]pyridin-2-yl)methyl)pyrrolidine-2,5-dione

Prepared from 1-[[7-[1-(azetidin-3-yl)-6-chloro-3,4-dihydro-2H-quinolin-8-yl]thieno[3,2-b]pyridin-2-yl]methyl]pyrrolidine-2,5-dione (Example 1, Step 7) and oxetan-3-one following the procedure described in the synthesis of Example 2. The final compound was purified by RP-HPLC. ¹H NMR (400 MHz, CD₃OD) δ=8.68 (dd, J=4.8, 0.9 Hz, 1H), 7.42-7.51 (m, 2H), 7.14 (s, 2H), 4.93 (s, 4H), 4.57 (t, J=6.8 Hz, 2H), 4.19 (t, J=5.6 Hz, 2H), 3.58-3.70 (m, 2H), 3.41 (s, 4H), 2.87 (t, J=6.4 Hz, 2H), 2.75 (s, 4H), 1.79-1.92 ppm (m, 2H). LCMS R_{T}=0.807 min, m/z=523.1 [M+H]+.

### Example 7: 3-[6-chloro-8-[2-[(2,5-dioxopyrrolidin-1-yl)methyl]thieno[3,2-b]pyridin-7-yl]-3,4-dihydro-2H-quinolin-1-yl]-N-methyl-azetidine-1-carboxamide

To a solution of 1-[[7-[1-(azetidin-3-yl)-6-chloro-3,4-dihydro-2H-quinolin-8-yl]thieno[3,2-b]pyridin-2-yl]methyl]pyrrolidine-2,5-dione (Example 1, Step 7, 30 mg, 0.06 mmol,) and triethylamine (6 mg, 0.06 mmol) in dichloromethane (1 mL) was added N-methylcarbamoyl chloride (18 mg, 0.19 mmol) at 0° C. After stirring at 25° C. for 1 h, the mixture was concentrated under reduced pressure. The residue was purified by RP-HPLC to afford Example 7. ¹H NMR (400 MHz, CD₃OD) δ=8.71 (d, J=4.8 Hz, 1H), 7.53-7.36 (m, 2H), 7.27-7.09 (m, 2H), 4.96 (s, 2H), 4.63 (s, 2H), 3.81-3.73 (m, 1H), 3.51-3.22 (m, 4H), 2.90 (t, J=6.4 Hz, 2H), 2.77 (s, 4H), 2.58 (s, 3H), 2.01-1.88 (m, 2H). LCMS R_{T}=1.042 min, m/z=524.1 [M+H]+.

### Example 8: 1-((7-(1-(1-acetylazetidin-3-yl)-6-chloro-1,2,3,4-tetrahydroquinolin-8-yl)thieno[3,2-b]pyridin-2-yl)methyl)pyrrolidine-2,5-dione

Prepared from 1-[[7-[1-(azetidin-3-yl)-6-chloro-3,4-dihydro-2H-quinolin-8-yl]thieno[3,2-b]pyridin-2-yl]methyl]pyrrolidine-2,5-dione (Example 1, Step 7,) and acetyl chloride following the procedure described in the synthesis of Example 7. The final compound was purified by RP-HPLC. ¹H NMR (400 MHz, CD₃OD) δ=8.70 (d, J=4.8 Hz, 1H), 7.38-7.53 (m, 2H), 7.14 (s, 2H), 4.91-5.00 (m, 4H), 3.77-3.84 (m, 1H), 3.32-3.73 (m, 4H), 2.88 (brt, J=6.4 Hz, 2H), 2.75 (s, 4H), 1.85-2.02 (m, 2H), 1.66 (s, 3H). LCMS R_{T}=1.708 min, m/z=509.1 [M+H]⁺

### Example 9: 3-[6-chloro-8-[2-[(2,5-dioxopyrrolidin-1-yl)methyl]thieno[3,2-b]pyridin-7-yl]-3,4-dihydro-2H-quinolin-1-yl]azetidine-1-carboxylate

Prepared from 1-[[7-[1-(azetidin-3-yl)-6-chloro-3,4-dihydro-2H-quinolin-8-yl]thieno[3,2-b]pyridin-2-yl]methyl]pyrrolidine-2,5-dione (Example 1, Step 7) and methyl chloroformate following the procedure described in the synthesis of Example 7. The final compound was purified by RP-HPLC. ¹H NMR (400 MHz, CD₃OD) δ=8.71 (d, J=4.9 Hz, 1H), 7.64-7.40 (m, 2H), 7.15 (q, J=2.5 Hz, 2H), 4.96 (s, 2H), 3.84-3.74 (m, 1H), 3.53 (s, 3H), 3.48-3.22 (m, 6H), 2.89 (brt, J=6.4 Hz, 2H), 2.76 (s, 4H), 1.95 (q, J=6.0 Hz, 2H). LCMS R_{T}=1.708 min, m/z=525.2 [M+H]⁺

### Example 10: 1-[[7-[6-chloro-1-[1-(2-hydroxyethyl)azetidin-3-yl]-3,4-dihydro-2H-quinolin-8-yl]thieno[3,2-b]pyridin-2-yl]methyl]pyrrolidine-2,5-dione, formic acid salt

To a solution of 1-[[7-[1-(azetidin-3-yl)-6-chloro-3,4-dihydro-2H-quinolin-8-yl]thieno[3,2-b]pyridin-2-yl]methyl]pyrrolidine-2,5-dione (Example 1, Step 7, 200 mg, 0.43 mmol) in acetonitrile (5 mL) was added 2-bromoethanol (64 mg, 0.51 mmol) and potassium carbonate (178 mg, 1.28 mmol) at 25° C. After stirring at 50° C. for 4 h, the mixture was concentrated under reduced pressure to remove solvent. The residue was purified by RP-HPLC to afford Example 10. ¹H NMR (400 MHz, CD₃OD) δ=8.72 (d, J=4.8 Hz, 1H), 8.47 (s, 1H), 7.44-7.51 (m, 2H), 7.18 (s, 2H), 4.94 (s, 2H), 3.83 (s, 1H), 3.49 (d, J=3.6 Hz, 6H), 3.31-3.33 (m, 2H), 2.90 (s, 4H), 2.76 (s, 4H), 1.77-1.94 (m, 2H). LCMS R_{T}=1.116 min, m/z=511.1 [M+H]⁺.

### Example 11: 1-((7-(6-chloro-1-(1-(2,2-difluoroethyl)azetidin-3-yl)-1,2,3,4-tetrahydroquinolin-8-yl)thieno[3,2-b]pyridin-2-yl)methyl)pyrrolidine-2,5-dione

To a solution of 1-((7-(1-(azetidin-3-yl)-6-chloro-1,2,3,4-tetrahydroquinolin-8-yl)thieno[3,2-b]pyridin-2-yl)methyl)pyrrolidine-2,5-dione (Example 1, Step 7, 150 mg, 0.32 mmol), potassium carbonate (133 mg, 0.96 mmol) and potassium iodide (5 mg, 0.03 mmol) in acetonitrile (5 mL) was added 2,2-difluoroethyl trifluoromethanesulfonate (103 mg, 0.48 mmol). After stirring at 60° C. for 20 min, the reaction was diluted with water (25 mL) and extracted with ethyl acetate (20 mL×3). The combined organic layers were washed with brine (30 mL), dried over anhydrous sodium sulfate, filtered and concentrated under reduced pressure. The residue was purified by RP-HPLC to afford Example 11. ¹H NMR (400 MHz, CD₃OD) δ=8.67 (d, J=4.8 Hz, 1H), 7.52-7.40 (m, 2H), 7.14 (d, J=1.6 Hz, 2H), 5.78-5.43 (m, 1H), 4.93 (s, 2H), 3.55 (q, J=7.6 Hz, 1H), 3.42 (s, 2H), 3.19-2.15 (m, 12H), 1.90-1.75 (m, 2H). LCMS R_{T}=0.638 min, m/z=531.5 [M+H]⁺.

### Example 12: 1-((3-(6-chloro-8-(2-((2,5-dioxopyrrolidin-1-yl)methyl)thieno[3,2-b]pyridin-7-yl)-3,4-dihydroquinolin-1(2H)-yl)azetidin-1-yl)methyl)cyclopropanecarbonitrile

To a solution of 1-[[7-[1-(azetidin-3-yl)-6-chloro-3,4-dihydro-2H-quinolin-8-yl]thieno[3,2-b]pyridin-2-yl]methyl]pyrrolidine-2,5-dione (Example 1, Step 7, 70 mg, 0.15 mmol) in acetonitrile (10 mL) was added (1-cyanocyclopropyl)methyl methanesulfonate (26.3 mg, 0.15 mmol) and potassium carbonate (62 mg, 0.45 mmol) at 25° C. After stirring for 2 h at 70° C., the reaction was quenched by addition of water (10 ml) and extracted with ethyl acetate (10 mL×2). The combined organic layers were washed with brine (20 mL), dried over anhydrous sodium sulfate, filtered and concentrated under reduced pressure. The residue was purified by RP-HPLC to afford Example 12. ¹H NMR (400 MHz, CD₃OD) δ=8.69 (d, J=4.8 Hz, 1H), 7.39-7.57 (m, 2H), 7.07-7.22 (m, 2H), 4.93 (s, 4H), 3.59 (q, J=7.6 Hz, 1H), 3.31-3.53 (m, 4H), 2.87 (t, J=6.4 Hz, 2H), 2.75 (s, 4H), 2.38 (s, 2H), 1.85 (s, 2H), 1.03-1.14 (m, 2H), 0.75-0.87 (m, 2H). LCMS R_{T}=1.258 min, m/z=546.2 [M+H]⁺

### Example 13: 3-((7-(1-(azetidin-3-yl)-6-chloro-1,2,3,4-tetrahydroquinolin-8-yl)thieno[3,2-b]pyridin-2-yl)methyl)-3-azabicyclo[3.1.0]hexane-2,4-dione, formic acid salt

Prepared from 3-azabicyclo[3.1.0]hexane-2,4-dione and tert-butyl 3-(6-chloro-8-(2-(hydroxymethyl)thieno[3,2-b]pyridin-7-yl)-3,4-dihydroquinolin-1(2H)-yl)azetidine-1-carboxylate (1.1) following the procedure described in the synthesis of Example 1. The final compound was purified by RP-HPLC. ¹H NMR (400 MHz, CD₃OD) δ=8.75 (d, J=4.8 Hz, 1H), 8.51 (br s, 1H), 7.51 (d, J=4.8 Hz, 1H), 7.44 (s, 1H), 7.27-7.10 (m, 2H), 4.82 (s, 2H), 4.02 (t, J=8.4 Hz, 1H), 3.50 (d, J=1.2 Hz, 2H), 3.47-3.21 (m, 4H), 2.93 (t, J=6.4 Hz, 2H), 2.62 (dd, J=3.6, 8.0 Hz, 2H), 1.93 (s, 2H), 1.65 (dt, J=4.4, 8.0 Hz, 1H), 1.45 (q, J=4.0 Hz, 1H). LCMS R_{T}=1.522 min, m/z=479.2 [M+H]⁺

### Example 14: 3-[[7-[1-(azetidin-3-yl)-6-chloro-3,4-dihydro-2H-quinolin-8-yl]thieno[3,2-b]pyridin-2-yl]methyl]-6,6-dimethyl-3-azabicyclo[3.1.0]hexane-2,4-dione, formic acid salt

Prepared from 6,6-dimethyl-3-azabicyclo[3.1.0]hexane-2,4-dione and tert-butyl 3-(6-chloro-8-(2-(hydroxymethyl)thieno[3,2-b]pyridin-7-yl)-3,4-dihydroquinolin-1(2H)-yl)azetidine-1-carboxylate (1.1) following the procedure described in the synthesis of Example 1. The final compound was purified by RP-HPLC. ¹H NMR (400 MHz, CD₃OD) δ=8.75 (d, J=4.8 Hz, 1H), 8.54 (s, 1H), 7.62-7.42 (m, 2H), 7.21 (d, J=6.0 Hz, 2H), 4.86 (s, 2H), 4.06-3.96 (m, 1H), 3.49 (br s, 2H), 3.47-3.21 (m, 4H), 2.94 (t, J=6.0 Hz, 2H), 2.53 (s, 2H), 1.93 (br s, 2H), 1.27 (s, 3H), 1.13 (s, 3H). LCMS R_{T}=1.663 min, m/z=507.2 [M+H]⁺

### Example 15: 3-[[7-[1-(azetidin-3-yl)-6-chloro-3,4-dihydro-2H-quinolin-8-yl]thieno[3,2-b]pyridin-2-yl]methyl]-1-methyl-pyrimidine-2,4-dione

Prepared from 1-methylpyrimidine-2,4-dione and tert-butyl 3-[6-chloro-8-[2-(hydroxymethyl)-thieno[3,2-b]pyridin-7-yl]-3,4-dihydro-2H-quinolin-1-yl]azetidine-1-carboxylate (1.1) following the procedure described in the synthesis of Example 1. The final compound was purified by RP-HPLC. ¹H NMR (400 MHz, CD₃OD) δ=8.69 (d, J=4.8 Hz, 1H), 7.60 (d, J=7.6 Hz, 1H), 7.53 (s, 1H), 7.47 (d, J=5.2 Hz, 1H), 7.19-7.14 (m, 2H), 5.78 (d, J=7.8 Hz, 1H), 5.39 (s, 2H), 3.84 (m, 1H), 3.47 (s, 2H), 3.42-3.34 (m, 7H), 2.90 (t, J=6.4 Hz, 2H), 1.95-1.85 (m, 2H). LCMS R_{T}=1.090 min, m/z=494.1 [M+H]⁺

### Example 16: 2-[[7-[1-(azetidin-3-yl)-6-chloro-3,4-dihydro-2H-quinolin-8-yl]thieno[3,2-b]pyridin-2-yl]methyl]pyridazin-3-one, formic acid salt

Prepared from 1H-pyridazin-6-one and tert-butyl 3-(6-chloro-8-(2-(hydroxymethyl)thieno[3,2-b]pyridin-7-yl)-3,4-dihydroquinolin-1(2H)-yl)azetidine-1-carboxylate (1.1) following the procedure described in the synthesis of Example 1. The final compound was purified by RP-HPLC. ¹H NMR (400 MHz, CD₃OD) δ=8.70 (d, J=4.8 Hz, 1H), 8.51 (s, 1H), 7.99 (dd, J=1.6, 3.6 Hz, 1H), 7.55 (s, 1H), 7.50-7.38 (m, 2H), 7.19-7.10 (m, 2H), 7.04 (dd, J=1.6, 9.2 Hz, 1H), 5.64 (s, 2H), 3.79 (q, J=8.0 Hz, 1H), 3.42 (br s, 2H), 3.47-3.21 (m, 4H), 2.88 (t, J=6.4 Hz, 2H), 1.96-1.82 (m, 2H). LCMS R_{T}=1.487 min, m/z=464.1 [M+H]⁺

### Example 17: 2-[[7-[1-(azetidin-3-yl)-6-chloro-3,4-dihydro-2H-quinolin-8-yl]thieno[3,2-b]pyridin-2-yl]methyl]-6-methyl-pyridazin-3-one, formic acid salt

Prepared from 3-methyl-1H-pyridazin-6-one and tert-butyl 3-[6-chloro-8-[2-(hydroxymethyl)-thieno[3,2-b]pyridin-7-yl]-3,4-dihydro-2H-quinolin-1-yl]azetidine-1-carboxylate (1.1) following the procedure described in the synthesis of Example 1. The final compound was purified by RP-HPLC. ¹H NMR (400 MHz, CD₃OD) δ=8.73 (d, J=5.2 Hz, 1H), 8.52 (s, 1H), 7.54 (s, 1H), 7.49 (d, J=4.8 Hz, 1H), 7.41 (d, J=9.6 Hz, 1H), 7.19 (s, 2H), 6.96 (d, J=9.2 Hz, 1H), 5.58 (s, 2H), 4.63 (s, 4H), 3.98 (t, J=8.4 Hz, 1H), 3.44 (s, 2H), 2.90 (t, J=6.4 Hz, 2H), 2.36 (s, 3H), 1.90 (t, J=5.2 Hz, 2H). LCMS R_{T}=1.090 min, m/z=478.1 [M+H]⁺

### Example 18: 2-[[7-[1-(azetidin-3-yl)-6-chloro-3,4-dihydro-2H-quinolin-8-yl]thieno[3,2-b]pyridin-2-yl]methyl]-4-methyl-pyridazin-3-one, formic acid salt

Prepared from 5-methyl-1H-pyridazin-6-one and tert-butyl 3-[6-chloro-8-[2-(hydroxymethyl)-thieno[3,2-b]pyridin-7-yl]-3,4-dihydro-2H-quinolin-1-yl]azetidine-1-carboxylate (1.1) following the procedure described in the synthesis of Example 1. The final compound was purified by RP-HPLC. ¹H NMR (400 MHz, CD₃OD) δ=8.72 (d, J=4.8 Hz, 1H), 8.51 (s, 1H), 7.85 (d, J=4.0 Hz, 1H), 7.54 (s, 1H), 7.48 (d, J=4.8 Hz, 1H), 7.28-7.34 (m, 1H), 7.18 (s, 2H), 5.63 (s, 2H), 4.31-4.81 (m, 2H), 3.89-4.27 (m, 2H), 3.37-3.56 (m, 3H), 2.89 (t, J=6.4 Hz, 2H), 2.20 (d, J=1.0 Hz, 3H), 1.89 (d, J=5.6 Hz, 2H). LCMS R_{T}=1.561 min, m/z=478.2 [M+H]⁺

### Example 19: 3-[[7-[1-(azetidin-3-yl)-6-chloro-3,4-dihydro-2H-quinolin-8-yl]thieno[3,2-b]pyridin-2-yl]methyl]quinazolin-4-one, formic acid salt

Prepared from 3H-quinazolin-4-one and tert-butyl 3-(6-chloro-8-(2-(hydroxymethyl)thieno[3,2-b]pyridin-7-yl)-3,4-dihydroquinolin-1(2H)-yl)azetidine-1-carboxylate (1.1) following the procedure described in the synthesis of Example 1. The final compound was purified by RP-HPLC. ¹H NMR (400 MHz, CD₃OD) δ=8.76 (d, J=4.8 Hz, 1H), 8.61-8.50 (m, 2H), 8.32 (dd, J=1.2, 8.0 Hz, 1H), 7.96-7.85 (m, 1H), 7.76 (d, J=8.0 Hz, 1H), 7.70-7.57 (m, 2H), 7.51 (d, J=4.8 Hz, 1H), 7.29-7.08 (m, 2H), 5.60 (s, 2H), 3.95-3.90 (m, 1H), 3.47-3.21 (m, 6H), 2.87 (brt, J=6.4 Hz, 2H), 1.93-1.68 (m, 2H). LCMS R_{T}=1.694 min, m/z=514.2 [M+H]⁺

### Example 20: 3-[[7-[1-(azetidin-3-yl)-6-chloro-3,4-dihydro-2H-quinolin-8-yl]thieno[3,2-b]pyridin-2-yl]methyl]pyrimidin-4-one, formic acid salt

Prepared from 1H-pyrimidin-6-one and tert-butyl 3-[6-chloro-8-[2-(hydroxymethyl)thieno[3,2-b]pyridin-7-yl]-3,4-dihydro-2H-quinolin-1-yl]azetidine-1-carboxylate (1.1) following the procedure described in the synthesis of Example 1. The final compound was purified by RP-HPLC. ¹H NMR (400 MHz, CD₃OD) δ=8.74 (d, J=4.8 Hz, 1H), 8.66 (bs, 1H), 7.99 (d, J=6.4 Hz, 1H), 8.50 (s, 1H), 7.60 (s, 1H), 7.50 (d, J=4.8 Hz, 1H), 7.17 (s, 2H), 6.54 (d, J=6.4 Hz, 1H), 5.50 (s, 2H), 3.98 (t, J=8.0 Hz, 1H), 3.50 (br s, 1H), 3.47-3.21 (m, 4H), 2.89 (t, J=6.4 Hz, 2H), 2.42 (br s, 1H), 1.89 (s, 2H). LCMS R_{T}=1.412 min, m/z=464.1 [M+H]⁺.

### Example 21: 2-[[7-[1-(azetidin-3-yl)-6-chloro-3,4-dihydro-2H-quinolin-8-yl]thieno[3,2-b]pyridin-2-yl]methyl]phthalazin-1-one, formic acid salt

Prepared from phthalazin-1(2H)-one and tert-butyl 3-(6-chloro-8-(2-(hydroxymethyl)thieno[3,2-b]pyridin-7-yl)-3,4-dihydroquinolin-1(2H)-yl)azetidine-1-carboxylate (1.1) following the procedure described in the synthesis of Example 1. The final compound was purified by RP-HPLC. ¹H NMR (400 MHz, CD₃OD) δ=8.72 (d, J=4.4 Hz, 1H), 8.50 (s, 1H), 8.43 (s, 1H), 8.38 (d, J=7.6 Hz, 1H), 8.00-7.83 (m, 3H), 7.58 (s, 1H), 7.52-7.42 (m, 1H), 7.20-7.13 (m, 2H), 5.70 (s, 2H), 4.78-4.50 (m, 2H), 3.96 (s, 1H), 3.82-3.61 (m, 2H), 3.49 (d, J=8.8 Hz, 1H), 2.85 (t, J=6.0 Hz, 2H), 2.42 (br s, 1H), 1.81 (s, 2H). LCMS R_{T}=1.790 min, m/z=514.1 [M+H].

### Example 22: 2-[[7-[1-(azetidin-3-yl)-6-chloro-3,4-dihydro-2H-quinolin-8-yl]thieno[3,2-b]pyridin-2-yl]methyl]-5-methyl-pyridazin-3-one, formic acid salt

Prepared from 4-methyl-1H-pyridazin-6-one and tert-butyl 3-[6-chloro-8-[2-(hydroxymethyl)-thieno[3,2-b]pyridin-7-yl]-3,4-dihydro-2H-quinolin-1-yl]azetidine-1-carboxylate (1.1) following the procedure described in the synthesis of Example 1. The final compound was purified by RP-HPLC. ¹H NMR (400 MHz, CD₃OD) δ=8.74 (d, J=4.4 Hz, 1H), 8.52 (s, 1H), 7.90 (s, 1H), 7.55 (s, 1H), 7.49 (d, J=4.3 Hz, 1H), 7.18 (s, 2H), 6.83 (s, 1H), 5.61 (s, 2H), 4.16 (s, 1H), 4.00 (s, 1H), 3.52-3.39 (m, 4H), 2.99-2.83 (m, 2H), 2.28 (br s, 4H), 1.90 (s, 2H). LCMS R_{T}=1.672 min, m/z=478.1 [M+H]⁺

### Example 23: 3-[[7-[1-(azetidin-3-yl)-6-chloro-3,4-dihydro-2H-quinolin-8-yl]thieno[3,2-b]pyridin-2-yl]methyl]-1-(2,2-difluoroethyl)pyrimidine-2,4-dione, formic acid salt

Prepared from 1-(2,2-difluoroethyl)pyrimidine-2,4-dione and tert-butyl 3-(6-chloro-8-(2-(hydroxymethyl)thieno[3,2-b]pyridin-7-yl)-3,4-dihydroquinolin-1(2H)-yl)azetidine-1-carboxylate (1.1) following the procedure described in the synthesis of Example 1. The final compound was purified by RP-HPLC. ¹H NMR (400 MHz, CD₃OD) δ=8.73 (br d, J=4.8 Hz, 1H), 8.52 (br s, 1H), 7.63 (d, J=7.9 Hz, 1H), 7.57-7.43 (m, 2H), 7.25-7.10 (m, 2H), 6.37-5.99 (m, 1H), 5.85 (d, J=8.0 Hz, 1H), 5.39 (br s, 2H), 4.00 (q, J=8.4 Hz, 1H), 3.48 (br s, 4H), 3.45-3.15 (m, 4H), 2.91 (br s, 1H), 1.91 (br s, 2H). LCMS R_{T}=1.464 min, m/z=544.1 [M+H]⁺

### Example 24: 3-[[7-[1-(azetidin-3-yl)-6-chloro-3,4-dihydro-2H-quinolin-8-yl]thieno[3,2-b]pyridin-2-yl]methyl]-1-(2,2,2-trifluoroethyl)pyrimidine-2,4-dione, formic acid salt

Prepared from 1-(2,2,2-trifluoroethyl)pyrimidine-2,4-dione and tert-butyl 3-(6-chloro-8-(2-(hydroxymethyl)thieno[3,2-b]pyridin-7-yl)-3,4-dihydroquinolin-1(2H)-yl)azetidine-1-carboxylate (1.1) following the procedure described in the synthesis of Example 1. The final compound was purified by RP-HPLC. ¹H NMR (400 MHz, CD₃OD) δ=8.74 (d, J=4.8 Hz, 1H), 8.52 (s, 1H), 7.68 (d, J=8.0 Hz, 1H), 7.58-7.42 (m, 2H), 7.21 (s, 2H), 5.91 (d, J=8.0 Hz, 1H), 5.41 (s, 2H), 4.74-4.52 (m, 2H), 4.02 (br t, J=8.4 Hz, 1H), 3.84-3.37 (m, 4H), 3.27-2.15 (m, 3H), 1.93 (br d, J=5.2 Hz, 2H). LCMS R_{T}=1.688 min, m/z=562.1 [M+H]⁺

### Example 25: 7-[1-(azetidin-3-yl)-6-chloro-3,4-dihydro-2H-quinolin-8-yl]-2-(1H-pyrazol-5-yloxymethyl)thieno[3,2-b]pyridine

To a solution of tert-butyl 3-[6-chloro-8-[2-(hydroxymethyl)thieno[3,2-b]pyridin-7-yl]-3,4-dihydro-2H-quinolin-1-yl]azetidine-1-carboxylate (1.1; 100 mg, 0.21 mmol) in toluene (1 mL) was added 1H-pyrazol-5-ol (35 mg, 0.41 mmol) and 2-(tributyl-phosphanylidene)acetonitrile (99 mg, 0.41 mmol) at 0° C. After stirring at 110° C. for 1 h, the reaction mixture was concentrated under reduced pressure. The residue was dissolved hydrochloric acid (4 M in dioxane, 1 mL). After stirring at 20° C. for 10 min, the reaction was concentrated under reduced pressure. The residue was purified by RP-HPLC to afford Example 25. ¹H NMR (400 MHz, CD₃OD) δ=9.04 (d, J=6.0 Hz, 1H), 8.09 (d, J=6.0 Hz, 1H), 7.84-7.96 (m, 2H), 7.29-7.40 (m, 2H), 6.21 (d, J=2.8 Hz, 1H), 5.82 (s, 2H), 3.75-4.40 (m, 4H), 3.41-3.68 (m, 3H), 2.95 (t, J=6.4 Hz, 2H), 1.87-2.05 (m, 2H). LCMS R_{T}=1.007 min, m/z=451.9 [M+H]⁺

### Example 26: 4-[[7-[1-(azetidin-3-yl)-6-chloro-3,4-dihydro-2H-quinolin-8-yl]thieno[3,2-b]pyridin-2-yl]methyl]-1H-1,2,4-triazol-5-one, formic acid salt

To a solution of tert-butyl 3-[6-chloro-8-[2-(hydroxymethyl)thieno[3,2-b]pyridin-7-yl]-3,4-dihydro-2H-quinolin-1-yl]azetidine-1-carboxylate (1.1; 100 mg, 0.21 mmol) in toluene (1 mL) was added 1H-1,2,4-triazol-5(4H)-one (35 mg, 0.41 mmol) and 2-(tributyl-phosphanylidene)acetonitrile (99 mg, 0.41 mmol) at 0° C. After stirring at 110° C. for 1 h, the reaction mixture was concentrated under reduced pressure. The residue was dissolved hydrochloric acid (4 M in dioxane, 1 mL) and stirred at 20° C. for 1 hr. The reaction mixture was concentrated under reduced pressure and the residue was purified by RP-HPLC to afford Example 26. ¹H NMR (400 MHz, CD₃OD) δ=8.75 (d, J=4.8 Hz, 1H), 8.50 (s, 1H), 7.94 (s, 1H), 7.49-7.55 (m, 2H), 7.18 (d, J=1.5 Hz, 2H), 5.21 (s, 2H), 3.55-4.51 (m, 4H), 3.45 (s, 3H), 2.89 (t, J=6.2 Hz, 2H), 1.89 (s, 2H). LCMS R_{T}=0.951 min, m/z=453.0 [M+H]⁺

### Example 27: 3-[[7-[1-(azetidin-3-yl)-6-chloro-3,4-dihydro-2H-quinolin-8-yl]thieno[3,2-b]pyridin-2-yl]methyl]oxazolidine-2,4-dione, formic acid salt

To a solution of tert-butyl 3-[6-chloro-8-[2-(hydroxymethyl)thieno[3,2-b]pyridin-7-yl]-3,4-dihydro-2H-quinolin-1-yl]azetidine-1-carboxylate (1.1; 100 mg, 0.21 mmol) in toluene (0.5 mL) was added oxazolidine-2,4-dione (42 mg, 0.41 mmol), tetramethylazodicarboxamide (71 mg, 0.41 mmol) and tributylphosphane (164 mg, 0.81 mmol) at 0° C. After stirring at 110° C. for 1.5 h, the reaction was concentrated under reduced pressure. The residue was dissolved hydrochloric acid (4 M in dioxane, 1 mL). After stirring at 20° C. for 1 h, the reaction mixture was concentrated under reduced pressure. The residue was purified by RP-HPLC to afford Example 27. ¹H NMR (400 MHz, CD3OD) δ=8.75 (d, J=4.8 Hz, 1H), 8.50 (s, 1H), 7.55 (s, 1H), 7.51 (d, J=4.8 Hz, 1H), 7.19 (s, 2H), 5.01 (s, 2H), 4.88-4.95 (m, 2H), 3.55-4.74 (m, 4H), 3.48 (s, 3H), 2.91 (t, J=6.4 Hz, 2H), 1.90 (s, 2H). LCMS R_{T}=0.727 min, m/z=469.2 [M+H]⁺

### Example 28: 1-[[7-[1-(azetidin-3-yl)-6-chloro-3,4-dihydro-2H-quinolin-8-yl]thieno[3,2-b]pyridin-2-yl]methyl]piperazine-2,6-dione, formic acid salt

To a solution of tert-butyl 3-[6-chloro-8-[2-(hydroxymethyl)thieno[3,2-b]pyridin-7-yl]-3,4-dihydro-2H-quinolin-1-yl]azetidine-1-carboxylate (1.1; 100 mg, 0.21 mmol) in toluene (1 mL) was added tert-butyl 3,5-dioxopiperazine-1-carboxylate (88 mg, 0.41 mmol), tributylphosphane (164 mg, 810.66 μmol) and tetramethylazodicarboxamide (71 mg, 0.41 mmol) at 0° C. After stirring at 110° C. for 1 h, the reaction was concentrated under reduced pressure. The residue was dissolved hydrochloric acid (4 M in dioxane, 1 mL). After stirring at 25° C. for 1 h, the reaction was concentrated under reduced pressure. The residue was purified by RP-HPLC to afford Example 28. ¹H NMR (400 MHz, CD₃OD) δ=8.72 (d, J=4.8 Hz, 1H), 8.50 (s, 1H), 7.44-7.51 (m, 2H), 7.15-0.22 (m, 2H), 5.21 (s, 2H), 3.70-4.50 (m, 4H), 3.32-3.69 (m, 7H), 2.91 (t, J=6.4 Hz, 2H), 1.92 (s, 2H). LCMS R_{T}=0.925 min, m/z=481.9 [M+H]⁺

### Example 29: 1-((7-(1-(azetidin-3-yl)-6-chloro-1,2,3,4-tetrahydroquinolin-8-yl)thieno[3,2-b]pyridin-2-yl)methyl)-4-((1-methylcyclopropyl)methyl)piperazine-2,6-dione, formic acid salt

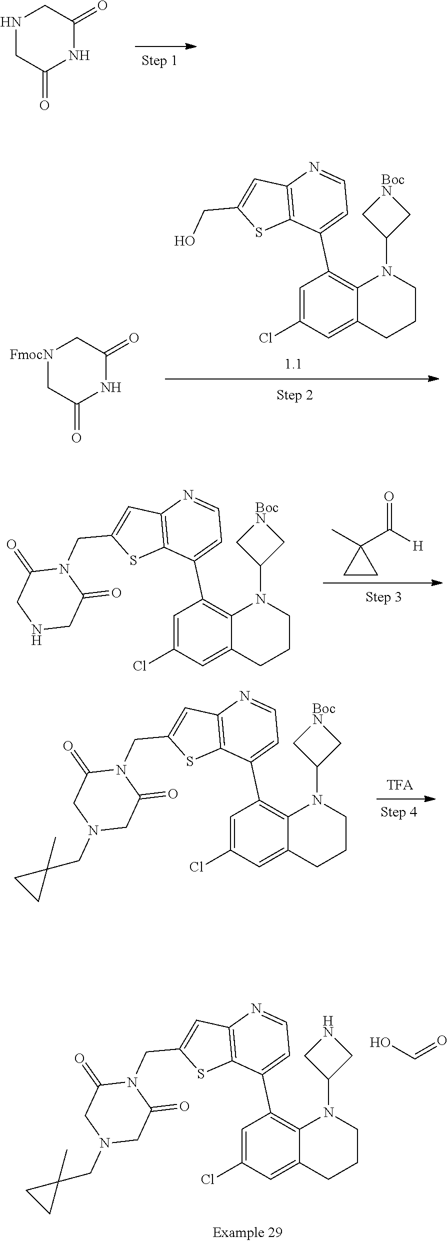

### Step 1: (9H-fluoren-9-yl)methyl 3,5-dioxopiperazine-1-carboxylate

To a solution of piperazine-2,6-dione (300 mg, 2.63 mmol) and N-ethyl-N-isopropylpropan-2-amine (407 mg, 3.16 mmol) in dichloromethane (8 mL) was added (9H-fluoren-9-yl)methyl carbonochloridate (816 mg, 3.16 mmol) at 0° C. After stirring at 0° C. for 1.5 h and 20° C. for 0.5 h, the reaction was diluted with water (25 mL) and extracted with dichloromethane (15 mL×2). The combined organic layers were washed with brine (20 mL), dried over anhydrous sodium sulfate, filtered and concentrated under reduced pressure. The residue was purified by column chromatography to afford the title compound. ¹H NMR (400 MHz, DMSO-d₆) δ=11.38 (s, 1H), 7.89 (d, J=7.6 Hz, 2H), 7.62 (d, J=7.2 Hz, 2H), 7.48-7.26 (m, 4H), 4.44 (d, J=6.4 Hz, 2H), 4.37-4.25 (m, 1H), 4.13 (s, 4H).

### Step 2: tert-butyl 3-(6-chloro-8-(2-((2,6-dioxopiperazin-1-yl)methyl)thieno[3,2-b]pyridin-7-yl)-3,4-dihydroquinolin-1(2H)-yl)azetidine-1-carboxylate

To a solution of tert-butyl 3-(6-chloro-8-(2-(hydroxymethyl)thieno[3,2-b]pyridin-7-yl)-3,4-dihydroquinolin-1(2H)-yl)azetidine-1-carboxylate (240 mg, 0.49 mmol), (9H-fluoren-9-yl)methyl 3,5-dioxopiperazine-1-carboxylate (249 mg, 0.74 mmol) and triphenylphosphine (388 mg, 1.48 mmol) in tetrahydrofuran (7 mL) was added diisopropyl azodicarboxylate (299 mg, 1.48 mmol). After stirring at 20° C. for 1 h, the reaction was concentrated under reduced pressure. The residue was purified by column chromatography to afford the title compound. ¹H NMR (400 MHz, CDCl₃) δ=8.71 (d, J=4.8 Hz, 1H), 7.57 (s, 1H), 7.23 (d, J=4.8 Hz, 1H), 7.13-7.10 (m, 1H), 7.07 (d, J=2.4 Hz, 1H), 5.21 (s, 2H), 3.74 (s, 3H), 3.66-3.62 (m, 2H), 3.49 (s, 4H), 3.40 (s, 2H), 2.85 (d, J=6.0 Hz, 2H), 1.96-1.89 (m, 2H), 1.34 (s, 9H). LCMS R_{T}=2.040 min, m/z=582.1 [M+H]⁺.

### Step 3: tert-butyl 3-(6-chloro-8-(2-((4-((1-methylcyclopropyl)methyl)-2,6-dioxopiperazin-1-yl)methyl)thieno[3,2-b]pyridin-7-yl)-3,4-dihydroquinolin-1(2H)-yl)azetidine-1-carboxylate

To a solution of 1-methylcyclopropanecarbaldehyde (30 mg, 0.36 mmol) in dichloromethane (1.5 mL) and dichloroethane (1.5 mL) was added tert-butyl 3-(6-chloro-8-(2-((2,6-dioxopiperazin-1-yl)methyl)thieno[3,2-b]pyridin-7-yl)-3,4-dihydroquinolin-1(2H)-yl)azetidine-1-carboxylate (110 mg, 0.19 mmol), acetic acid (113 mg, 1.89 mmol) and sodium triacetoxyborohydride (120 mg, 0.57 mmol). After stirring at 20° C. for 1 h, the reaction was quenched by addition of saturated aqueous sodium bicarbonate (0.3 mL), dried over anhydrous sodium sulfate and concentrated under reduced pressure. The residue was purified by prep-TLC to afford the title compound.

### Step 4

A solution of tert-butyl 3-(6-chloro-8-(2-((4-((1-methylcyclopropyl)methyl)-2,6-dioxopiperazin-1-yl)methyl)thieno[3,2-b]pyridin-7-yl)-3,4-dihydroquinolin-1(2H)-yl)azetidine-1-carboxylate (25 mg, 0.038 mmol) in 1,1,1,3,3,3-hexafluoropropan-2-ol (1 mL) and trifluoroacetic acid (44 mg, 0.38 mmol) was stirred at 20° C. for 3 h. Then the reaction was concentrated under reduced pressure. The residue was purified by RP-HPLC to afford Example 29. ¹H NMR (400 MHz, CD₃OD) δ=8.72 (d, J=4.8 Hz, 1H), 8.51 (s, 1H), 7.52-7.42 (m, 2H), 7.23-7.12 (m, 2H), 5.22 (s, 2H), 4.59-3.36 (m, 10H), 3.00-2.20 (m, 5H), 1.90 (s, 2H), 1.06 (s, 3H), 0.40-0.20 (m, 4H). LCMS R_{T}=1.803 min, m/z=550.1 [M+H]⁺.

### Example 30: 3-[[7-[1-(azetidin-3-yl)-6-chloro-3,4-dihydro-2H-quinolin-8-yl]thieno[3,2-b]pyridin-2-yl]methyl]-1-(2-methoxyethyl)pyrimidine-2,4-dione

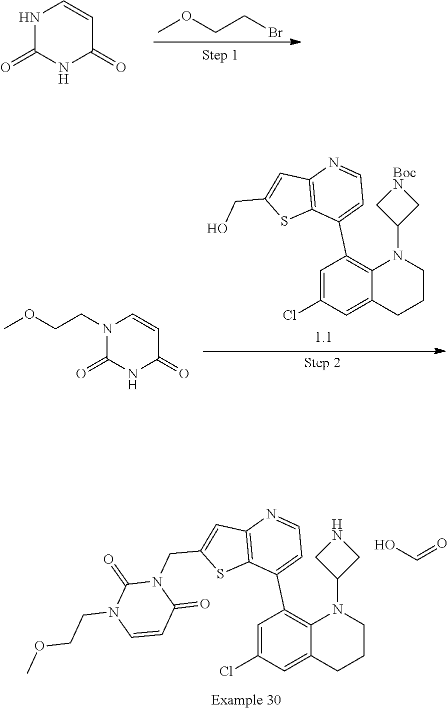

### Step 1: 1-(2-methoxyethyl)pyrimidine-2,4-dione

To a solution of 1H-pyrimidine-2,4-dione (2.0 g, 17.84 mmol) and 1-bromo-2-methoxy-ethane (2.5 g, 17.84 mmol) in N,N-dimethylformamide (30 mL) was added sodium hydride (856 mg, 21.41 mmol, 60% in mineral oil) at 20° C. under nitrogen atmosphere. After stirring at 40° C. for 16 h, the mixture was quenched by addition of water (150 mL) and extracted with ethyl acetate (30 mL×7). The combined organic layers were washed with brine (220 mL), dried over anhydrous sodium sulfate, filtered and concentrated under reduced pressure. The residue was purified by column chromatography to give 1-(2-methoxyethyl)pyrimidine-2,4-dione. ¹H NMR (400 MHz, CD₃OD) δ=11.24 (s, 1H), 7.55 (d, J=8.0 Hz, 1H), 5.51-5.53 (m, 1H), 3.82 (t, J=5.2 Hz, 2H), 3.50 (t, J=5.2 Hz, 2H), 3.25 (s, 3H). Step 2:

To a solution of 1-(2-methoxyethyl)pyrimidine-2,4-dione (49 mg, 0.29 mmol) in tetrahydrofuran (1 mL) was added tert-butyl 3-[6-chloro-8-[2-(hydroxymethyl)thieno[3,2-b]pyridin-7-yl]-3,4-dihydro-2H-quinolin-1-yl]azetidine-1-carboxylate (1.1; 70 mg, 0.14 mmol), triphenylphosphine (76 mg, 0.29 mmol) and diisopropyl azodicarboxylate (73 mg, 0.36 mmol) at 0° C. After stirring at 40° C. for 1 h, the reaction mixture was concentrated under reduced pressure. The residue was dissolved hydrochloric acid (4 M in dioxane, 1 mL). After stirring at 20° C. for 30 min., the reaction was concentrated under reduced pressure. The residue was purified by RP-HPLC to afford Example 30. ¹H NMR (400 MHz, CD₃OD) δ=8.71 (d, J=4.8 Hz, 1H), 8.48 (s, 1H), 7.58 (d, J=8.0 Hz, 1H), 7.52 (s, 1H), 7.46 (d, J=4.8 Hz, 1H), 7.18 (s, 2H), 5.75 (d, J=8.0 Hz, 1H), 5.39 (s, 2H), 3.90-4.42 (m, 4H), 3.33-3.90 (m, 6H), 3.14-3.30 (m, 4H), 2.90 (t, J=6.4 Hz, 2H), 1.91 (d, J=4.8 Hz, 2H). LCMS R_{T}=1.363 min, m/z=538.2 [M+H]⁺

### Example 31: 7-((7-(1-(azetidin-3-yl)-6-chloro-1,2,3,4-tetrahydroquinolin-8-yl)thieno[3,2-b]pyridin-2-yl)methyl)-5-methyl-5,7-diazaspiro[3.4]octane-6,8-dione, formic acid salt

Prepared from 5-methyl-5,7-diazaspiro[3.4]octane-6,8-dione and tert-butyl 3-[6-chloro-8-[2-(hydroxymethyl)thieno[3,2-b]pyridin-7-yl]-3,4-dihydro-2H-quinolin-1-yl]azetidine-1-carboxylate (1.1) following the procedure described in the synthesis of Example 1. The final compound was purified by RP-HPLC. ¹H NMR (400 MHz, CD₃OD) δ=8.75 (d, J=4.8 Hz, 1H), 8.52 (s, 1H), 7.62-7.42 (m, 2H), 7.22 (s, 2H), 4.98 (s, 2H), 4.17-3.93 (m, 2H), 3.48 (s, 3H), 3.39-3.38 (m, 1H), 3.07 (s, 3H), 2.92 (t, J=6.0 Hz, 2H), 2.66 (m, 2H), 2.42-2.30 (m, 2H), 2.26-2.11 (m, 1H), 2.00-1.83 (m, 4H). LCMS R_{T}=0.805 min, m/z=522.2 [M+H]⁺.

### Example 32: 1-(azetidin-3-yl)-8-[2-[(2,5-dioxopyrrolidin-1-yl)methyl]thieno[3,2-b]pyridin-7-yl]-3,4-dihydro-2H-quinoline-6-carbonitrile, formic acid salt

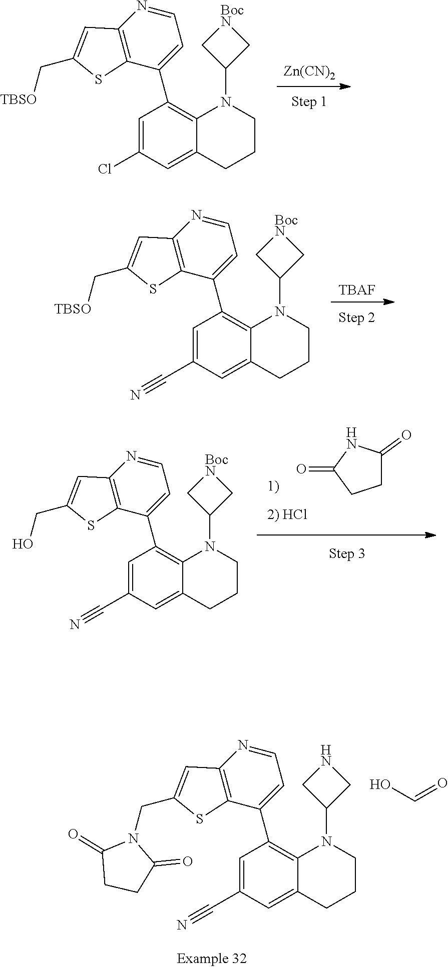

### Step 1: tert-butyl 3-[8-[2-[[tert-butyl(dimethyl)silyl]oxymethyl]thieno[3,2-b]pyridin-7-yl]-6-cyano-3,4-dihydro-2H-quinolin-1-yl]azetidine-1-carboxylate

To a solution of tert-butyl 3-[8-[2-[[tert-butyl(dimethyl)silyl]oxymethyl]thieno[3,2-b]pyridin-7-yl]-6-chloro-3,4-dihydro-2H-quinolin-1-yl]azetidine-1-carboxylate (Example 1, Step 5; 100 mg, 0.17 mmol) and zinc cyanide (196 mg, 1.67 mmol) in N-methyl pyrrolidone (1 mL) was added bis(tri-tert-butylphosphine) palladium(0) (42 mg, 0.083 mmol) at 25° C. After stirring at 150° C. for 30 min under microwave, the reaction was quenched by addition of water (10 mL), then extracted with ethyl acetate (10 mL×3). The combined organic layers were washed with brine (20 mL), dried over anhydrous sodium sulfate, filtered and concentrated under reduced pressure to afford the title compound. LCMS R_{T}=1.062 min, m/z=591.2 [M+H]⁺

### Step 2: tert-butyl 3-[6-cyano-8-[2-(hydroxymethyl)thieno[3,2-b]pyridin-7-yl]-3,4-dihydro-2H-quinolin-1-yl]azetidine-1-carboxylate

To a solution of tert-butyl 3-[8-[2-[[tert-butyl(dimethyl)silyl]oxymethyl]thieno[3,2-b]pyridin-7-yl]-6-cyano-3,4-dihydro-2H-quinolin-1-yl]azetidine-1-carboxylate (80 mg, 0.14 mmol) in tetrahydrofuran (5 mL) was added tetrabutylammonium fluoride (0.1 mL, 1 M in tetrahydrofuran) at 25° C. After stirring at 25° C. for 30 min, the reaction was concentrated under reduced pressure. The residue was purified by prep-TLC to afford the title compound. LCMS R_{T}=0.829 min, m/z=477.2 [M+H]⁺

### Step 3

Prepared from succinimide and tert-butyl 3-[6-cyano-8-[2-(hydroxymethyl)thieno[3,2-b]pyridin-7-yl]-3,4-dihydro-2H-quinolin-1-yl]azetidine-1-carboxylate following the procedure described in the synthesis of Example 1. The final compound was purified by RP-HPLC to afford Example 32. ¹H NMR (400 MHz, CD₃OD) δ=8.78 (d, J=4.8 Hz, 1H), 8.53 (br s, 1H), 7.62-7.45 (m, 4H), 4.97 (s, 2H), 4.27 (s, 1H), 4.15 (q, J=8.4 Hz, 1H), 3.70-3.47 (m, 4H), 3.01-2.88 (m, 2H), 2.78 (s, 4H), 2.34 (br s, 1H), 2.04 (q, J=5.6 Hz, 2H). LCMS R_{T}=1.334 min, m/z=458.2 [M+H]⁺

### Example 33: 1-[[7-[1-(azetidin-3-yl)-6-methyl-3,4-dihydro-2H-quinolin-8-yl]thieno[3,2-b]pyridin-2-yl]methyl]pyrrolidine-2,5-dione, formic acid salt

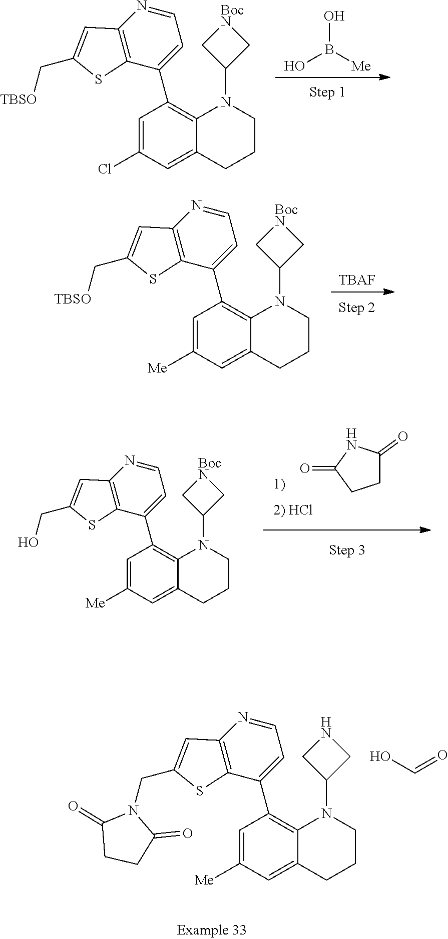

### Step 1: tert-butyl 3-[8-[2-[[tert-butyl(dimethyl)silyl]oxymethyl]thieno[3,2-b]pyridin-7-yl]-6-methyl-3,4-dihydro-2H-quinolin-1-yl]azetidine-1-carboxylate

To a solution of tert-butyl 3-[8-[2-[[tert-butyl(dimethyl)silyl]oxymethyl]thieno[3,2-b]pyridin-7-yl]-6-chloro-3,4-dihydro-2H-quinolin-1-yl]azetidine-1-carboxylate (Example 1, Step 5; 100 mg, 0.17 mmol) in dioxane (1 mL) and water (0.1 mL) was added methylboronic acid (15 mg, 0.25 mmol), cesium carbonate (163 mg, 0.50 mmol) and XPhos Pd G₃ (14 mg, 0.02 mmol) at 25° C. After stirring at 100° C. for 15 h, the reaction mixture was filtered and the filtrate was concentrated under reduced pressure. The residue was purified by prep-TLC to afford the title compound. LCMS R_{T}=1.087 min, m/z=580.2 [M+H]⁺.

### Step 2: tert-butyl 3-[8-[2-(hydroxymethyl)thieno[3,2-b]pyridin-7-yl]-6-methyl-3,4-dihydro-2H-quinolin-1-yl]azetidine-1-carboxylate

To a solution of tert-butyl 3-[8-[2-[[tert-butyl(dimethyl)silyl]oxymethyl]thieno[3,2-b]pyridin-7-yl]-6-methyl-3,4-dihydro-2H-quinolin-1-yl]azetidine-1-carboxylate (88 mg, 0.15 mmol) in tetrahydrofuran (1 mL) was added tetrabutylammonium fluoride (0.03 mL, 1 M in tetrahydrofuran) at 25° C. After stirring for 1 h at 25° C., the mixture was concentrated under reduced pressure to remove solvent. The residue was purified by prep-TLC to afford the title compound. LCMS R_{T}=0.859 min, m/z=466.2 [M+H]⁺.

### Step 3

Prepared from succinimide and tert-butyl 3-[8-[2-(hydroxymethyl)thieno[3,2-b]pyridin-7-yl]-6-methyl-3,4-dihydro-2H-quinolin-1-yl]azetidine-1-carboxylate following the procedure described in the synthesis of Example 1. The final compound was purified by RP-HPLC to afford Example 33. ¹H NMR (400 MHz, CD₃OD) δ=8.62-8.71 (m, 1H), 8.49 (s, 1H), 7.44 (s, 2H), 6.98 (s, 2H), 4.92 (s, 2H), 3.62-4.12 (m, 3H), 3.45 (s, 2H), 3.26-3.34 (m, 1H), 3.23-3.38 (m, 1H), 2.86 (d, J=5.6 Hz, 2H), 2.76 (d, J=2.4 Hz, 4H), 2.26 (s, 3H), 1.87 (s, 2H). LCMS R_{T}=0.679 min, m/z=447.2 [M+H]⁺

### Example 34: 2-[[7-[1-(azetidin-3-yl)-6-chloro-3,4-dihydro-2H-quinolin-8-yl]thieno[3,2-b]pyridin-2-yl]methyl]-6-methyl-pyridazin-3-one, hydrochloride salt

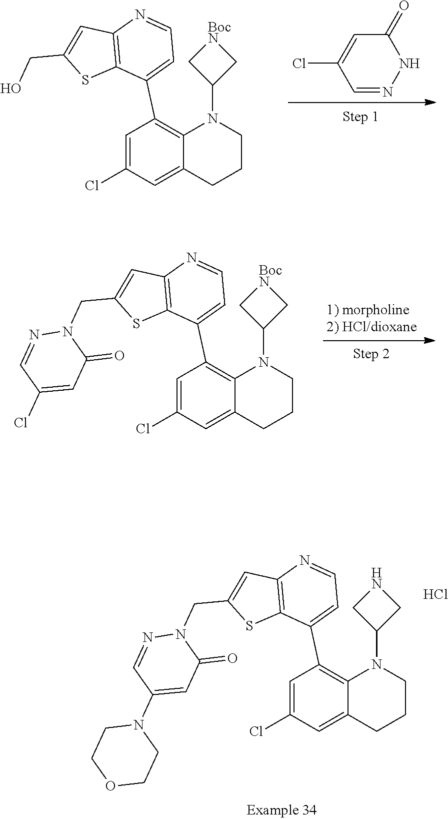 [SMILES: CC(C)(C)OC(=O)N1CC(N2CCCc3cc(Cl)cc(-c4ccnc5cc(CO)sc45)c32)C1 ; O=c1cc(Cl)cn[nH]1 ; CC(C)(C)OC(=O)N1CC(N2CCCc3cc(Cl)cc(-c4ccnc5cc(Cn6ncc(Cl)cc6=O)sc45)c32)C1 ; O=c1cc(N2CCOCC2)cnn1Cc1cc2nccc(-c3cc(Cl)cc4c3N(C3CNC3)CCC4)c2s1 ; Cl]

### Step 1: tert-butyl 3-[6-chloro-8-[2-[(4-chloro-6-oxo-pyridazin-1-yl)methyl]thieno[3,2-b]pyridin-7-yl]-3,4-dihydro-2H-quinolin-1-yl]azetidine-1-carboxylate

To a solution of tert-butyl 3-[6-chloro-8-[2-(hydroxymethyl)thieno[3,2-b]pyridin-7-yl]-3,4-dihydro-2H-quinolin-1-yl]azetidine-1-carboxylate (1.1; 400 mg, 0.82 mmol), 4-chloro-1H-pyridazin-6-one (215 mg, 1.65 mmol) and triphenylphosphine (432 mg, 1.65 mmol) in tetrahydrofuran (5 mL) was added diisopropyl azodicarboxylate (333 mg, 1.65 mmol) at 0° C. After stirring at 0° C. for 10 min, the resulting mixture was allowed to warm to 20° C. and stirred for 50 min. The mixture was concentrated under reduced pressure, and the residue was purified by column chromatography to afford the title compound. LCMS R_{T}=1.037 min, m/z=598.1 [M+H]⁺.

### Step 2

To a solution of tert-butyl 3-[6-chloro-8-[2-[(4-chloro-6-oxo-pyridazin-1-yl)methyl]thieno[3,2-b]pyridin-7-yl]-3,4-dihydro-2H-quinolin-1-yl]azetidine-1-carboxylate (200 mg, 0.17 mmol) in N,N-dimethylformamide (1 mL) were added morpholine (29 mg, 0.34 mmol) and potassium carbonate (35 mg, 0.25 mmol). After stirring at 60° C. for 7 h, the reaction was quenched by addition of water (20 mL) and extracted with ethyl acetate (5 mL×4). The combined organic layers were washed with brine (5 mL×4), dried over anhydrous sodium sulfate, filtered and concentrated under reduced pressure. The residue was purified by prep-TLC. The product was dissolved in dioxane (1 mL) and hydrochloric acid (8 mL, 4 M in dioxane) was added. After stirring at 20° C. for 60 min, the reaction mixture was concentrated under reduced pressure. The residue was purified by RP-HPLC (2-32% acetonitrile in water and 0.225% hydrogen chloride) to afford Example 34. ¹H NMR (400 MHz, CD₃OD) δ=9.01 (d, J=6.4 Hz, 1H), 8.14-8.02 (m, 2H), 7.81 (s, 1H), 7.34 (dd, J=2.4, 16.4 Hz, 2H), 5.99 (d, J=2.4 Hz, 1H), 5.67 (s, 2H), 4.16-3.93 (m, 2H), 3.90-3.69 (m, 5H), 3.65-3.36 (m, 7H), 3.17-2.85 (m, 3H), 2.01-1.89 (m, 2H). LCMS R_{T}=0.805 min, m/z=549.1 [M+H]⁺.

### Example 35: 2-[[7-[1-(azetidin-3-yl)-6-chloro-3,4-dihydro-2H-quinolin-8-yl]thieno[3,2-b]pyridin-2-yl]methyl]-5-(cyclobutylamino)pyridazin-3-one, formic acid salt

Prepared from cyclobutylamine and tert-butyl 3-[6-chloro-8-[2-[(4-chloro-6-oxo-pyridazin-1-yl)methyl]thieno[3,2-b]pyridin-7-yl]-3,4-dihydro-2H-quinolin-1-yl]azetidine-1-carboxylate (Example 34, Step 1) following the procedure described in the synthesis of Example 34. The final compound was purified by RP-HPLC. ¹H NMR (400 MHz, CD₃OD) δ=8.71 (d, J=5.2 Hz, 1H), 8.53 (s, 1H), 7.54 (d, J=2.4 Hz, 1H), 7.49-7.45 (m, 2H), 7.19 (d, J=4.4 Hz, 2H), 5.54 (d, J=2.4 Hz, 1H), 5.48 (s, 2H), 3.98-3.84 (m, 2H), 3.42 (s, 2H), 3.34-3.32 (m, 4H), 2.89 (t, J=6.4 Hz, 2H), 2.46-2.37 (m, 2H), 2.00-1.82 (m, 6H). LCMS R_{T}=0.770 min, m/z=533.2 [M+H]⁺.

### Example 36: 2-((7-(1-(azetidin-3-yl)-6-chloro-1,2,3,4-tetrahydroquinolin-8-yl)thieno[3,2-b]pyridin-2-yl)methyl)-5-(4-fluorophenoxy)pyridazin-3(2H)-one, formic acid salt

Prepared from 4-fluorophenol and tert-butyl 3-[6-chloro-8-[2-[(4-chloro-6-oxo-pyridazin-1-yl)methyl]thieno[3,2-b]pyridin-7-yl]-3,4-dihydro-2H-quinolin-1-yl]azetidine-1-carboxylate (Example 34, Step 1) following the procedure described in the synthesis of Example 34. The final compound was purified by RP-HPLC. ¹H NMR (400 MHz, CD₃OD) δ=8.74 (d, J=4.8 Hz, 1H), 8.49 (s, 1H), 8.01 (d, J=2.8 Hz, 1H), 7.42-7.60 (m, 2H), 7.11-7.32 (m, 6H), 5.92 (d, J=2.8 Hz, 1H), 5.59 (s, 2H), 3.94-4.07 (m, 1H), 3.45 (s, 2H), 3.31 (s, 4H), 2.90 (t, J=6.4 Hz, 2H), 1.89 (s, 2H). LCMS R_{T}=0.837 min, m/z=574.0 [M+H]⁺

### Example 37: 2-[[7-[1-(azetidin-3-yl)-6-chloro-3,4-dihydro-2H-quinolin-8-yl]thieno[3,2-b]pyridin-2-yl]methyl]pyrazol-3-ol

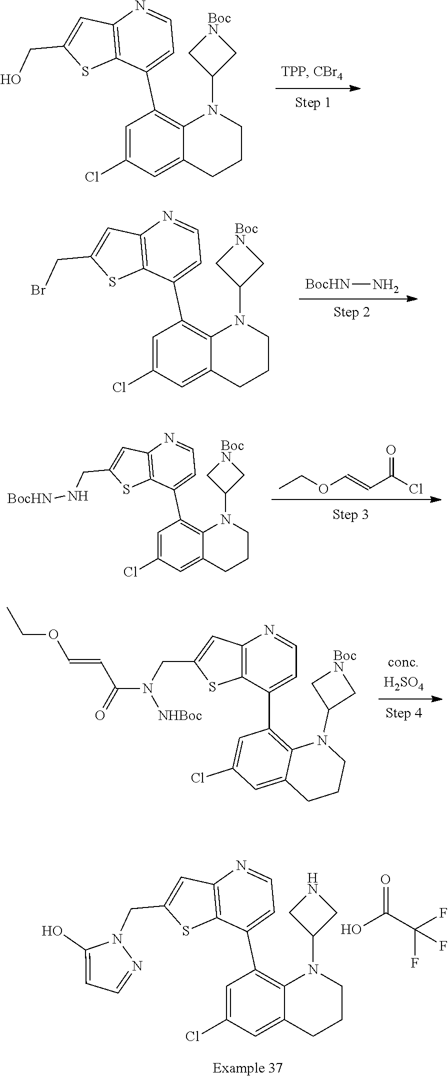

### Step 1: tert-butyl 3-[8-[2-(bromomethyl)thieno[3,2-b]pyridin-7-yl]-6-chloro-3,4-dihydro-2H-quinolin-1-yl]azetidine-1-carboxylate

To a solution of tert-butyl 3-[6-chloro-8-[2-(hydroxymethyl)thieno[3,2-b]pyridin-7-yl]-3,4-dihydro-2H-quinolin-1-yl]azetidine-1-carboxylate (1.1; 800 mg, 1.65 mmol) in dichloromethane (10 mL) were added triphenylphosphine (863 mg, 3.29 mmol) and carbon tetrabromide (819 mg, 2.47 mmol) at 0° C. After stirring at 25° C. for 6 h, the reaction mixture was filtered through a pad of celite and the filtrate was concentrated under reduced pressure. The residue was purified by column chromatography to afford the title compound. LCMS R_{T}=1.047 min, m/z=549.9 [M+H]⁺.

### Step 2: tert-butyl 3-[8-[2-[(2-tert-butoxycarbonylhydrazino)methyl]thieno[3,2-b]pyridin-7-yl]-6-chloro-3,4-dihydro-2H-quinolin-1-yl]azetidine-1-carboxylate

To a solution of tert-butyl 3-[8-[2-(bromomethyl)thieno[3,2-b]pyridin-7-yl]-6-chloro-3,4-dihydro-2H-quinolin-1-yl]azetidine-1-carboxylate (500 mg, 0.91 mmol) in N,N-dimethylformamide (6 mL) were added potassium carbonate (252 mg, 1.82 mmol) and tert-butyl N-aminocarbamate (181 mg, 1.37 mmol) at 25° C. After stirring at 25° C. for 16 h, the reaction mixture was filtered and the filtrate was concentrated under reduced pressure to afford the title compound. It was used in the next step without further purification. LCMS R_{T}=0.953 min, m/z=600.1 [M+H]⁺.

### Step 3: tert-butyl 3-[8-[2-[[(tert-butoxycarbonylamino)-[(E)-3-ethoxyprop-2-enoyl]amino]methyl]thieno[3,2-b]pyridin-7-yl]-6-chloro-3,4-dihydro-2H-quinolin-1-yl]azetidine-1-carboxylate

To a solution of tert-butyl 3-[8-[2-[(2-tert-butoxycarbonylhydrazino)methyl]thieno[3,2-b]pyridin-7-yl]-6-chloro-3,4-dihydro-2H-quinolin-1-yl]azetidine-1-carboxylate (400 mg, 0.67 mmol) in dichloromethane (10 mL) was added (E)-3-ethoxyprop-2-enoyl chloride (89 mg, 0.67 mmol) at 25° C. After stirring at 25° C. for 14 h, the reaction mixture was diluted with dichloromethane (20 mL) and washed with water (10 ml). The organic layer was dried over anhydrous sodium sulfate, filtered and concentrated under reduced pressure to afford the title compound. It was used in next step without further purification. LCMS RT=1.030 min, m/z=698.1 [M+H]⁺.

### Step 4

To a solution of tert-butyl 3-[8-[2-[[(tert-butoxycarbonylamino)-[(E)-3-ethoxyprop-2-enoyl]amino]methyl]thieno[3,2-b]pyridin-7-yl]-6-chloro-3,4-dihydro-2H-quinolin-1-yl]azetidine-1-carboxylate (300 mg, 0.43 mmol) in dichloromethane (3 mL) was added concentrated sulfuric acid (86 mg, 0.86 mmol) at 25° C. After stirring at 25° C. for 0.5 h, the reaction mixture was concentrated under reduced pressure. The residue was purified by RP-HPLC to afford Example 37. ¹H NMR (400 MHz, CD₃OD) δ=8.81 (d, J=2.0 Hz, 1H), 7.67-7.61 (m, 1H), 7.53 (s, 1H), 7.45 (s, 1H), 7.25 (d, J=5.6 Hz, 2H), 5.55-5.46 (m, 2H), 4.90-4.87 (m, 3H), 4.01 (q, J=8.4 Hz, 1H), 3.45 (s, 2H), 3.34-3.32 (m, 2H), 2.98-2.87 (m, 2H), 1.97-1.88 (m, 2H). LCMS R_{T}=0.745 min, m/z=452.1 [M+H]⁺.

### Example 38: Azetidin-1-yl-[7-[1-(azetidin-3-yl)-6-chloro-3,4-dihydro-2H-quinolin-8-yl]thieno[3,2-b]pyridin-2-yl]methanone, trifluoroacetic acid salt

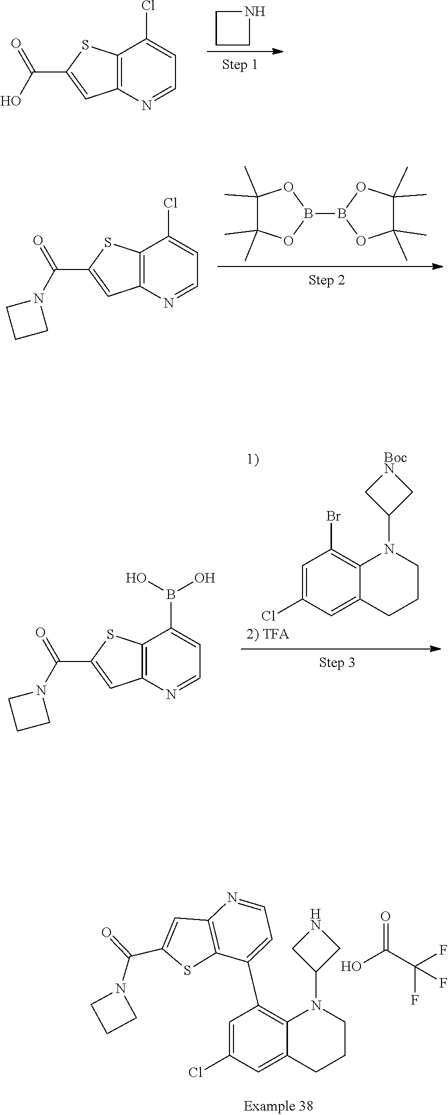

### Step 1: azetidin-1-yl-(7-chlorothieno[3,2-b]pyridin-2-yl)methanone

To a solution of 7-chlorothieno[3,2-b]pyridine-2-carboxylic acid (300 mg, 1.40 mmol), azetidine hydrochloride (263 mg, 2.81 mmol) and 2-(7-azabenzotriazol-1-yl)-N,N,N,N-tetramethyluronium hexafluorophosphate (641 mg, 1.69 mmol) in N,N-dimethylformamide (3 mL) was added N-ethyl-N-isopropylpropan-2-amine (726 mg, 5.62 mmol). After stirring at 25° C. for 7 h under nitrogen atmosphere, the reaction mixture was diluted with water (20 mL) and extracted with ethyl acetate (20 mL×2). The combined organic layers were washed with brine (20 mL), dried over anhydrous sodium sulfate, filtered and concentrated under reduced pressure. The residue was purified by column chromatography to afford the title compound. ¹H NMR (400 MHz, CD₃OD) δ=8.68 (d, J=5.2 Hz, 1H), 7.89 (s, 1H), 7.59 (d, J=5.2 Hz, 1H), 4.71 (t, J=7.6 Hz, 2H), 4.27 (t, J=7.6 Hz, 2H), 2.50 (q, J=7.6 Hz, 2H). LCMS R_{T}=1.481 min, m/z=252.9 [M+H]⁺.

### Step 2: [2-(azetidine-1-carbonyl)thieno[3,2-b]pyridin-7-yl]boronic acid

To a solution of azetidin-1-yl(7-chlorothieno[3,2-b]pyridin-2-yl)methanone (130 mg, 0.51 mmol), bis(pinacolato)diboron (261 mg 1.03 mmol) and potassium acetate (151 mg, 1.54 mmol) in dioxane (1 mL) was added dichloro[1,1′-bis(diphenylphosphino)ferrocene]palladium(II) (112 mg, 0.15 mmol) under nitrogen atmosphere. After stirring at 120° C. for 1 h, the reaction mixture was concentrated under reduced pressure. The residue was diluted with water (20 mL) and extracted with ethyl acetate (20 mL×2). The combined organic layers were washed with brine (20 mL), dried over anhydrous sodium sulfate, filtered and concentrated under reduced pressure to afford the title compound. It was used in next step without further purification. LCMS R_{T}=1.322 min, m/z=263.0 [M+H]⁺.

### Step 3

To a solution of tert-butyl 3-(8-bromo-6-chloro-3,4-dihydro-2H-quinolin-1-yl)azetidine-1-carboxylate (172 mg, 0.43 mmol), [2-(azetidine-1-carbonyl)thieno[3,2-b]pyridin-7-yl]boronic acid (135 mg, 0.52 mmol) and cesium carbonate (279 mg, 0.86 mmol) in dioxane (2 mL) and water (0.5 mL) was added dichloro[1,1′-bis(diphenylphosphino)ferrocene]palladium(II) (94 mg, 0.13 mmol) under nitrogen atmosphere. After stirring at 100° C. for 2 h, the reaction mixture was filtered and the filtrate was concentrated under reduced pressure. The residue was diluted with water (20 mL) and extracted with ethyl acetate (20 mL×2). The combined organic layers were washed with brine (20 mL), dried over anhydrous sodium sulfate, filtered and concentrated under reduced pressure. The residue was dissolved in dichloromethane (5 mL) and trifluoroacetic acid (770 mg, 6.75 mmol) was added. After stirring at 25° C. for 30 min, the reaction was concentrated under reduced pressure. The residue was purified by RP-HPLC to afford Example 38. ¹H NMR (400 MHz, CD₃OD) δ=8.86 (d, J=4.8 Hz, 1H), 7.89 (s, 1H), 7.63 (d, J=4.8 Hz, 1H), 7.23 (s, 2H), 4.68 (t, J=7.6 Hz, 2H), 4.38-3.42 (m, 8H), 2.93 (t, J=6.4 Hz, 2H), 2.78-2.09 (m, 3H), 1.91 (s, 2H). LCMS R_{T}=1.617 min, m/z=439.1 [M+H]⁺.

### Example 39: 1-((7-(1-(Azetidin-3-yl)-6-(trifluoromethyl)-1,2,3,4-tetrahydroquinolin-8-yl)-1H-inden-2-yl)methyl)pyrrolidine-2,5-dione, formic acid salt

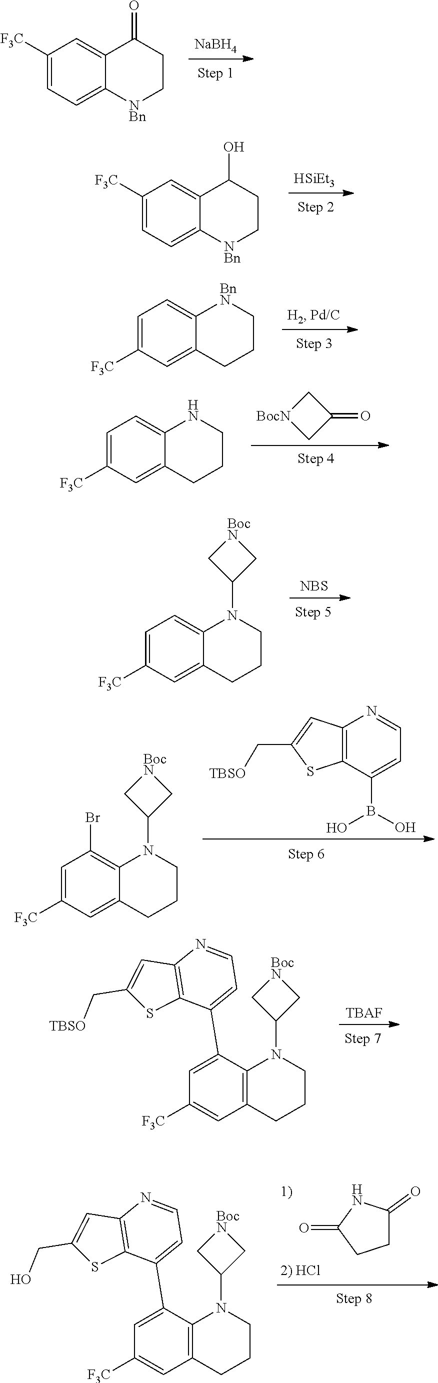

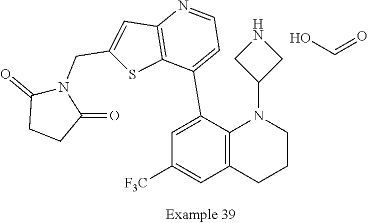

### Step 1: 1-benzyl-6-(trifluoromethyl)-1,2,3,4-tetrahydroquinolin-4-ol

To a solution of 1-benzyl-6-(trifluoromethyl)-2,3-dihydroquinolin-4(1H)-one (700 mg, 2.29 mmol) in methanol (20 mL) was added sodium borohydride (217 mg, 5.73 mmol) at 0° C. After stirring at 50° C. for 1 h under nitrogen atmosphere, the reaction mixture was quenched by addition of saturated aqueous ammonium chloride solution (10 mL) and extracted with ethyl acetate (20 mL×2). The combined organic layers were washed with brine (20 mL), dried over anhydrous sodium sulfate, filtered and concentrated under reduced pressure to afford the title compound. It was used in next step without further purification. LCMS R_{T}=2.309 min, m/z=307.9 [M+H]⁺.

### Step 2: 1-benzyl-6-(trifluoromethyl)-1,2,3,4-tetrahydroquinoline

To a solution of 1-benzyl-6-(trifluoromethyl)-1,2,3,4-tetrahydroquinolin-4-ol (1.05 g, 3.42 mmol) in dichloromethane (15 mL) were added triethylsilane (2.78 g, 23.92 mmol) and trifluoroacetic acid (3.90 g, 34.17 mmol). After stirring at 20° C. for 2.5 h under nitrogen atmosphere, the reaction mixture was concentrated under reduced pressure to afford the title compound. It was used in next step without further purification. LCMS R_{T}=1.469 min, m/z=292.2 [M+H]⁺.

### Step 3: 6-(trifluoromethyl)-1,2,3,4-tetrahydroquinoline

To a solution of 1-benzyl-6-(trifluoromethyl)-1,2,3,4-tetrahydroquinoline (900 mg, 3.09 mmol) in methanol (5 mL) was added 5% palladium on carbon (100 mg, 0.31 mmol) and the mixture was flushed with hydrogen. After stirring at 20° C. for 12 h under hydrogen atmosphere of 15 psi, the reaction was filtered, and the filtrate was concentrated under reduced pressure. The residue was purified by column chromatography to afford the title compound. It was used in next step without further purification. LCMS R_{T}=1.306 min, m/z=202.0 [M+H]⁺.

### Step 4: tert-butyl 3-(6-(trifluoromethyl)-3,4-dihydroquinolin-1(2H)-yl)azetidine-1-carboxylate

To the solution of 6-(trifluoromethyl)-1,2,3,4-tetrahydroquinoline (180 mg, 0.90 mmol) and tert-butyl 3-oxoazetidine-1-carboxylate (306 mg, 1.79 mmol) in acetic acid (5 mL) was added sodium triacetoxyborohydride (379 mg, 1.79 mmol). After stirring at 20° C. for 2 h under nitrogen atmosphere, the reaction mixture was concentrated under reduced pressure to afford the title compound. It was used in next step without further purification. LCMS R_{T}=1.552 min, m/z=357.0 [M+H]⁺.

### Step 5: tert-butyl 3-(8-bromo-6-(trifluoromethyl)-3,4-dihydroquinolin-1(2H)-yl)azetidine-1-carboxylate

To a solution of tert-butyl 3-(6-(trifluoromethyl)-3,4-dihydroquinolin-1(2H)-yl)azetidine-1-carboxylate (145 mg, 0.41 mmol) in acetonitrile (5 mL) was added N-bromosuccinimide (72 mg, 0.41 mmol). After stirring at 20° C. for 0.5 h, the reaction mixture was concentrated under reduced pressure. The residue was purified by prep-TLC to afford the title compound. LCMS R_{T}=1.507 min, m/z=436.9 [M+H]⁺.

### Step 6: tert-butyl 3-(8-(2-(((tert-butyldimethylsilyl)oxy)methyl)thieno[3,2-b]pyridin-7-yl)-6-(trifluoromethyl)-3,4-dihydroquinolin-1(2H)-yl)azetidine-1-carboxylate

To a solution of (2-(((tert-butyldimethylsilyl)oxy)methyl)thieno[3,2-b]pyridin-7-yl)boronic acid (Example 1, Step 4; 111 mg, 0.34 mmol), tert-butyl 3-(8-bromo-6-(trifluoromethyl)-3,4-dihydroquinolin-1(2H)-yl)azetidine-1-carboxylate (100 mg, 0.23 mmol) and potassium carbonate (63 mg, 0.46 mmol) in dioxane (2 mL) and water (0.03 mL) was added dichloro[1,1′-bis(diphenylphosphino)ferrocene]palladium(II) (34 mg, 0.05 mmol) under nitrogen atmosphere. After stirring at 140° C. for 1 h under nitrogen atmosphere, the reaction mixture was filtered and concentrated under reduced pressure. The residue was purified by prep-TLC to afford the title compound. LCMS R_{T}=1.139 m/z=634.2 [M+H]⁺.

### Step 7: tert-butyl 3-[8-[2-(hydroxymethyl)thieno[3,2-b]pyridin-7-yl]-6-(trifluoromethyl)-3,4-dihydro-2H-quinolin-1-yl]azetidine-1-carboxylate

To a solution of tert-butyl 3-[8-[2-[[tert-butyl(dimethyl)silyl]oxymethyl]thieno[3,2-b]pyridin-7-yl]-6-(trifluoromethyl)-3,4-dihydro-2H-quinolin-1-yl]azetidine-1-carboxylate (140 mg, 0.22 mol) in tetrahydrofuran (2 mL) was added tetrabutylammonium fluoride (1 M in tetrahydrofuran, 0.22 ml). After stirring at 25° C. for 0.5 h, the reaction mixture was concentrated under reduced pressure. The residue was purified by column chromatography to afford the title compound. LCMS R_{T}=0.908 min, m/z=520.2 [M+H]⁺

### Step 8

Example 39 was prepared from tert-butyl 3-[8-[2-(hydroxymethyl)thieno[3,2-b]pyridin-7-yl]-6-(trifluoromethyl)-3,4-dihydro-2H-quinolin-1-yl]azetidine-1-carboxylate (80 mg, 0.16 mmol) and succinimide (22.88 mg, 0.23 mmol), following the procedure described in the synthesis of Example 1. The final product was purified by RP-HPLC. ¹H NMR (400 MHz, CD₃OD) δ=8.77 (d, J=4.8 Hz, 1H), 8.50 (s, 1H), 7.55 (d, J=4.8 Hz, 1H), 7.50 (s, 1H), 7.45 (d, J=3.2 Hz, 2H), 4.96 (s, 2H), 4.37-4.04 (m, 2H), 3.81-3.44 (m, 4H), 2.98 (s, 2H), 2.83-2.68 (m, 4H), 2.01 (d, J=4.8 Hz, 1H), 2.50-1.90 (m, 1H), 2.12-1.89 (m, 1H). LCMS R_{T}=0.767 min, m/z=501.3 [M+H]⁺

### Example 40: 1-((7-(6-chloro-1-(oxetan-3-yl)-1,2,3,4-tetrahydroquinolin-8-yl)thieno[3,2-b]pyridin-2-yl)methyl)pyrrolidine-2,5-dione

Prepared from 6-chloro-1,2,3,4-tetrahydroquinoline and oxetan-3-one following the procedure described in the synthesis of Example 1. The final compound was purified by RP-HPLC. ¹H NMR (400 MHz, CD₃OD) δ=8.69 (d, J=4.8 Hz, 1H), 7.40-7.50 (m, 2H), 7.13 (s, 2H), 4.94 (s, 4H), 4.62 (s, 2H), 4.08 (s, 1H), 3.58 (s, 2H), 2.88 (t, J=6.4 Hz, 2H), 2.76 (s, 4H), 1.84-1.96 (m, 2H). LCMS R_{T}=1.797 min, m/z=468.1 [M+H]⁺

### Example 41: Cis-1-[[7-[6-chloro-1-(3-hydroxycyclobutyl)-3,4-dihydro-2H-quinolin-8-yl]thieno[3,2-b]pyridin-2-yl]methyl]pyrrolidine-2,5-dione

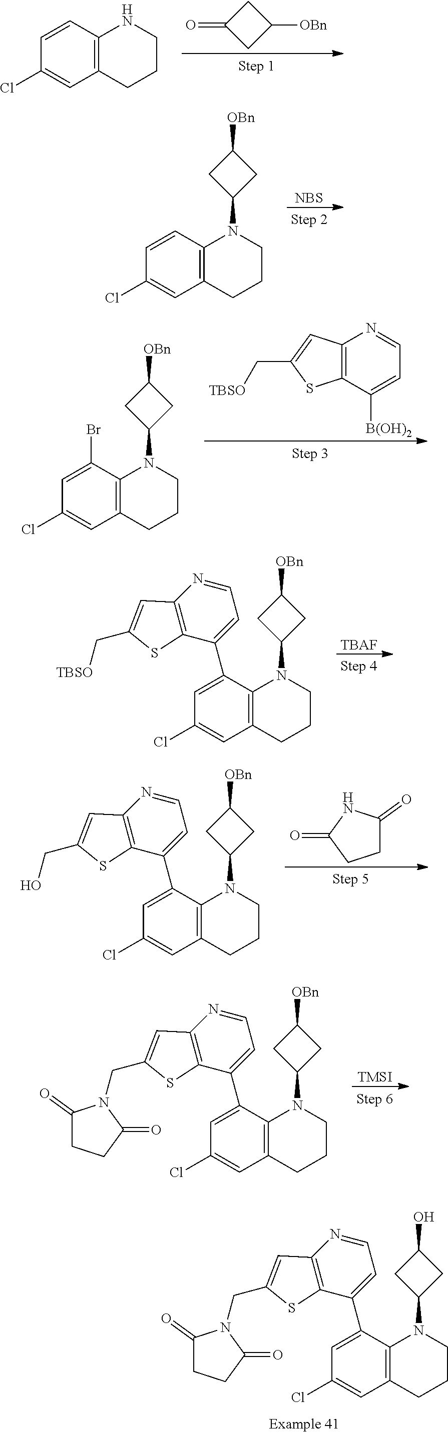

### Step 1: cis-1-(3-benzyloxycyclobutyl)-6-chloro-3,4-dihydro-2H-quinoline

To a solution of 6-chloro-1,2,3,4-tetrahydroquinoline (600 mg, 3.58 mmol) in tetrahydrofuran (20 mL) were added tetraisopropoxytitanium (2.03 g, 7.16 mmol), 3-benzyloxycyclobutanone (946 mg, 5.37 mmol) and sodium cyanoborohydride (449 mg, 7.16 mmol) at 25° C. After stirring at 25° C. for 20 h, the reaction was concentrated under reduced pressure. The residue was purified by RP-HPLC to afford the title compound. LCMS R_{T}=2.817 min, m/z=328.1 [M+H]⁺.

### Step 2: cis-tert-butyl 1-(3-benzyloxycyclobutyl)-8-bromo-6-chloro-3,4-dihydro-2H-quinoline

To a solution of 1-(3-benzyloxycyclobutyl)-6-chloro-3,4-dihydro-2H-quinoline (96 mg, 0.29 mmol) in N,N-dimethylformamide (1 mL) was added N-bromosuccinimide (78 mg, 0.44 mmol) at 0° C. After stirring for 1 h at 25° C., the reaction mixture was concentrated under reduced pressure. The residue was purified by prep-TLC to afford the title compound. LCMS R_{T}=1.224 min, m/z=408.0 [M+H]⁺.

### Step 3: cis-[7-[1-(3-benzyloxycyclobutyl)-6-chloro-3,4-dihydro-2H-quinolin-8-yl]thieno[3,2-b]pyridin-2-yl]methoxy-tert-butyl-dimethyl-silane

To a solution of 1-(3-benzyloxycyclobutyl)-8-bromo-6-chloro-3,4-dihydro-2H-quinoline (94 mg, 0.23 mmol) in dioxane (2 mL) and water (0.4 mL) were added [2-[[tert-butyl(dimethyl)silyl]oxymethyl]thieno[3,2-b]pyridin-7-yl]boronic acid (112 mg, 0.35 mmol), dichloro[1,1′-bis(diphenylphosphino)ferrocene]palladium(II) (51 mg, 0.07 mmol) and potassium carbonate (96 mg, 0.69 mmol). After stirring for 1 h at 100° C., the reaction mixture was filtered, and the filtrate was concentrated under reduced pressure. The residue was purified by prep-TLC to afford the title compound. LCMS R_{T}=1.157 min, m/z=605.1 [M+H]⁺.

### Step 4: cis-[7-[1-(3-benzyloxycyclobutyl)-6-chloro-3,4-dihydro-2H-quinolin-8-yl]thieno[3,2-b]pyridin-2-yl]methanol

To a solution [7-[1-(3-benzyloxycyclobutyl)-6-chloro-3,4-dihydro-2H-quinolin-8-yl]thieno[3,2-b]pyridin-2-yl]methoxy-tert-butyl-dimethyl-silane (60 mg, 0.1 mmol) in tetrahydrofuran (1 mL) was added tetrabutylammonium fluoride (1.0 M in tetrahydrofuran, 0.02 mL) at 25° C. After stirring for 30 min at 25° C., the reaction mixture was concentrated under reduced pressure. The residue was purified by prep-TLC to afford the title compound. LCMS R_{T}=0.921 min, m/z=491.1 [M+H]⁺

### Step 5: cis-1-[[7-[1-(3-benzyloxycyclobutyl)-6-chloro-3,4-dihydro-2H-quinolin-8-yl]thieno[3,2-b]pyridin-2-yl]methyl]pyrrolidine-2,5-dione

To a solution of [7-[1-(3-benzyloxycyclobutyl)-6-chloro-3,4-dihydro-2H-quinolin-8-yl]thieno[3,2-b]pyridin-2-yl]methanol (37 mg, 0.08 mmol), succinimide (15 mg, 0.15 mmol) and triphenylphosphine (39 mg, 0.15 mmol) in tetrahydrofuran (1 mL) was added diisopropyl azodicarboxylate (31 mg, 0.15 mmol) at 0° C. After stirring at 25° C. for 1 h, the reaction mixture was concentrated under reduced pressure. The residue was purified by RP-HPLC to afford the title compound. LCMS R_{T}=1.444 min, m/z=572.1 [M+H]⁺

### Step 6

To a solution of 1-[[7-[1-(3-benzyloxycyclobutyl)-6-chloro-3,4-dihydro-2H-quinolin-8-yl]thieno[3,2-b]pyridin-2-yl]methyl]pyrrolidine-2,5-dione (8 mg, 0.01 mmol) in methylene chloride (0.5 mL) was added iodo(trimethyl)silane (39 mg, 0.20 mmol) at 20° C. After stirring at 20° C. for 1 h, the reaction mixture was concentrated under reduced pressure. The residue was purified by RP-HPLC to afford Example 41. ¹H NMR (400 MHz, CD₃OD) δ=8.66-8.68 (m, 1H), 7.44-7.46 (m, 2H), 7.11-7.12 (m, 2H), 4.95 (s, 2H), 3.34-3.34 (m, 4H), 3.25-3.29 (m, 1H), 2.88 (t, J=6.4 Hz, 2H), 2.76-2.82 (m, 5H), 1.85-1.88 (m, 2H), 1.68 (s, 2H). LCMS R_{T}=1.043 min, m/z=482.1 [M+H]⁺

### Example 42a and Example 42b: cis-1-((7-(1-(3-aminocyclobutyl)-6-chloro-1,2,3,4-tetrahydroquinolin-8-yl)thieno[3,2-b]pyridin-2-yl)methyl)pyrrolidine-2,5-dione, formic acid salt and trans-1-((7-(1-(3-aminocyclobutyl)-6-chloro-1,2,3,4-tetrahydroquinolin-8-yl)thieno[3,2-b]pyridin-2-yl)methyl)pyrrolidine-2,5-dione, formic acid salt

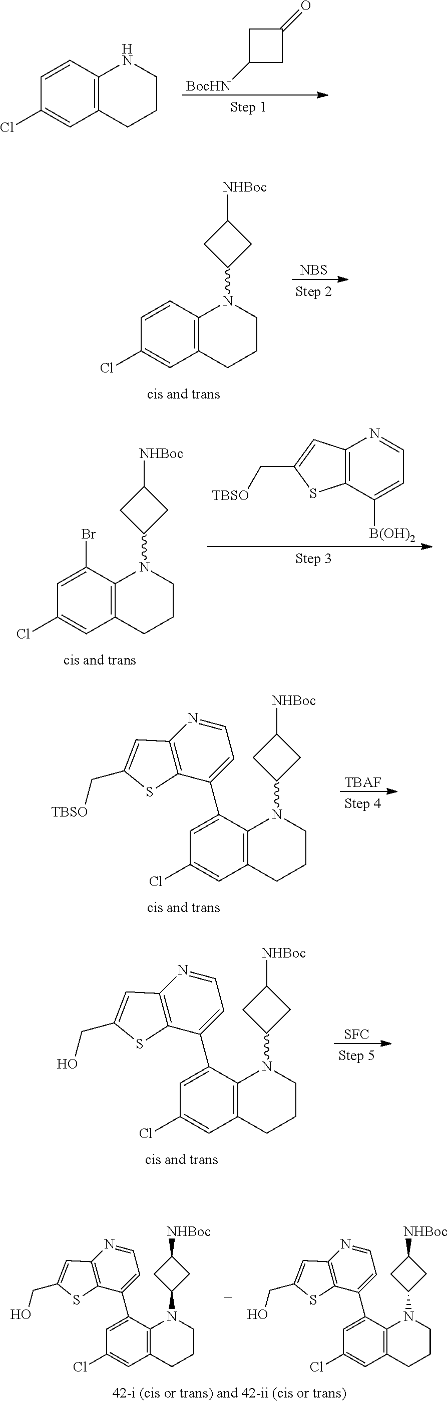

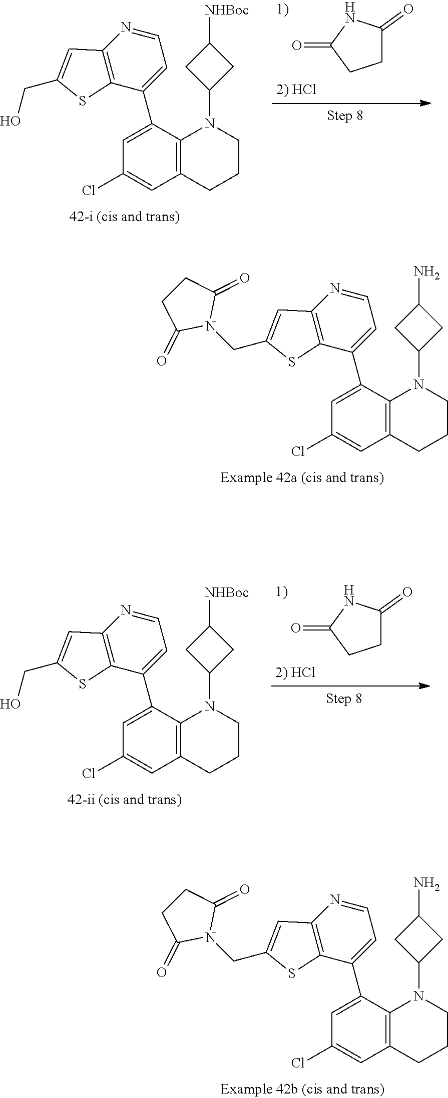 [SMILES: CC(C)(C)OC(=O)NC1CC(N2CCCc3cc(Cl)cc(-c4ccnc5cc(CO)sc45)c32)C1 ; O=C1CCC(=O)N1 ; NC1CC(N2CCCc3cc(Cl)cc(-c4ccnc5cc(CN6C(=O)CCC6=O)sc45)c32)C1 ; CC(C)(C)OC(=O)NC1CC(N2CCCc3cc(Cl)cc(-c4ccnc5cc(CO)sc45)c32)C1 ; O=C1CCC(=O)N1 ; NC1CC(N2CCCc3cc(Cl)cc(-c4ccnc5cc(CN6C(=O)CCC6=O)sc45)c32)C1]

### Step 1: cis- and trans-tert-butyl (3-(6-chloro-3,4-dihydroquinolin-1(2H)-yl)cyclobutyl)carbamate

To a solution of 6-chloro-1,2,3,4-tetrahydroquinoline (500 mg, 2.98 mmol) in dichloromethane (20 mL) were added tert-butyl N-(3-oxocyclobutyl)carbamate (1.1 g, 5.97 mmol), sodium triacetoxyborohydride (1.9 g, 8.95 mmol) and acetic acid (179 mg, 2.98 mmol). After stirring at 25° C. for 12 h, the mixture was filtered and concentrated under reduced pressure. The residue was purified by RP-HPLC to the title compound as a mixture of both cis and trans isomers. LCMS R_{T}=2.152 min, m/z=337.3 [M+H]⁺.

### Step 2: cis- and trans-tert-butyl (3-(8-bromo-6-chloro-3,4-dihydroquinolin-1(2H)-yl)cyclobutyl)carbamate

To a solution of tert-butyl N-[3-(6-chloro-3,4-dihydro-2H-quinolin-1-yl)cyclobutyl]carbamate (450 mg, 1.34 mmol) in N,N-dimethylformamide (3 mL) was added bromine (427 mg, 2.67 mmol) and iron (149 mg, 2.67 mmol) at 25° C. After stirring for 2 h at 0° C., the reaction was quenched by addition of saturated aqueous sodium bicarbonate (10 mL) and extracted with ethyl acetate (10 mL×3). The combined organic layers were washed with brine (20 mL), dried over anhydrous sodium sulfate, filtered and concentrated under reduced pressure. The residue was purified by column chromatography to afford the title compound as a mixture of both cis and trans isomers. LCMS R_{T}=1.113 min, m/z=417.0 [M+H]⁺.

### Step 3: cis- and trans-tert-butyl (3-(8-(2-(((tert-butyldimethylsilyl)oxy)methyl)thieno[3,2-b]pyridin-7-yl)-6-chloro-3,4-dihydroquinolin-1(2H)-yl)cyclobutyl)carbamate

A mixture of tert-butyl (3-(8-bromo-6-chloro-3,4-dihydroquinolin-1(2H)-yl)cyclobutyl)carbamate (530 mg, 1.27 mmol), [2-[[tert-butyl(dimethyl)silyl]oxymethyl]thieno[3,2-b]pyridin-7-yl]boronic acid (618 mg, 1.91 mmol), dichloro[1,1′-bis(diphenylphosphino)ferrocene]palladium(II) (186 mg, 254.96 mmol) and cesium carbonate (1.25 g, 3.82 mmol) in water (1 mL) and dioxane (10 mL) was stirred at 110° C. for 2 h. The reaction mixture was diluted with water (10 mL) and extracted with ethyl acetate (5 mL×2). The combined organic layers were washed with brine (5 mL), dried over anhydrous sodium sulfate, filtered and concentrated under reduced pressure. The residue was purified by column chromatography to afford the title compound as a mixture of both cis and trans isomers. LCMS R_{T}=1.162 min, m/z=614.4 [M+H]⁺

### Step 4: tert-butyl (3-(6-chloro-8-(2-(hydroxymethyl)thieno[3,2-b]pyridin-7-yl)-3,4-dihydroquinolin-1(2H)-yl)cyclobutyl)carbamate

To a solution of tert-butyl N-[3-[8-[2-[[tert-butyl(dimethyl)silyl]oxymethyl]thieno[3,2-b]pyridin-7-yl]-6-chloro-3,4-dihydro-2H-quinolin-1-yl]cyclobutyl]carbamate (630 mg, 1.03 mmol, 1 eq) in tetrahydrofuran (5 mL) was added tetrabutylammonium fluoride (1 M in tetrahydrofuran, 1 mL). After stirring at 20° C. for 1 h, the mixture was concentrated under reduced pressure. The residue was purified by prep-TLC to afford the title compound as a mixture of both cis and trans isomers. LCMS R_{T}=2.308 min, m/z=500.1 [M+H]⁺.

### Step 5: cis-tert-butyl 3-(6-chloro-8-(2-(hydroxymethyl)thieno[3,2-b]pyridin-7-yl)-3,4-dihydroquinolin-1(2H)-yl)cyclobutyl)carbamate and trans-tert-butyl 3-(6-chloro-8-(2-(hydroxymethyl)thieno[3,2-b]pyridin-7-yl)-3,4-dihydroquinolin-1(2H)-yl)cyclobutyl)carbamate

The above product (200 mg, 0.4 mmol) was separated by SFC to give first eluting fraction (42-i, Rt=3.812 min; LCMS R_{T}=2.320 min, m/z=500.1 [M+H]+) and second eluting fraction (42-ii, Rt=4.231 min; LCMS R_{T}=2.320 min, m/z=500.1 [M+H]+). (The cis/trans configuration of the isomers were not determined.)

### Step 6

Example 42a was prepared from 42-i and succinimide following the procedure described in the synthesis of Example 1. The final compound was purified by RP-HPLC. ¹H NMR (400 MHz, CD₃OD) δ=8.70 (d, J=4.8 Hz, 1H), 8.53 (s, 1H), 7.53-7.48 (m, 2H), 7.13 (q, J=2.4 Hz, 2H), 4.93 (s, 2H), 3.77-3.69 (m, 1H), 3.36-3.31 (m, 5H), 2.88-2.75 (m, 2H), 2.37-2.01 (m, 4H), 1.95-1.87 (m, 4H). LCMS R_{T}=0.767 min, m/z=481.2 [M+H]⁺. (The cis/trans configuration of the isomers were not determined.)

Example 42b was prepared from 42-ii and succinimide following the procedure described in the synthesis of Example 1. The final compound was purified by RP-HPLC. ¹H NMR (400 MHz, CD₃OD) δ=8.69 (d, J=4.8 Hz, 1H), 8.54 (s, 1H), 7.49-7.42 (m, 2H), 7.16 (s, 2H), 4.95 (s, 2H), 3.45-3.34 (m, 4H), 3.19-3.09 (m, 1H), 2.91 (t, J=6.4 Hz, 2H), 2.85-2.76 (m, 5H), 1.88-1.97 m, 4H). LCMS R_{T}=0.763 min, m/z=481.1 [M+H]⁺. (The cis/trans configuration of the isomers were not determined.)

### Example 43a and Example 43b: 1-((7-((1aS,7bR)-3-(azetidin-3-yl)-6-chloro-1a,2,3,7b-tetrahydro-1H-cyclopropa[c]quinolin-4-yl)thieno[3,2-b]pyridin-2-yl)methyl)pyrrolidine-2,5-dione, formic acid salt and 1-((7-((1aR,7bS)-3-(azetidin-3-yl)-6-chloro-1a,2,3,7b-tetrahydro-1H-cyclopropa[c]quinolin-4-yl)thieno[3,2-b]pyridin-2-yl)methyl)pyrrolidine-2,5-dione, formic acid salt

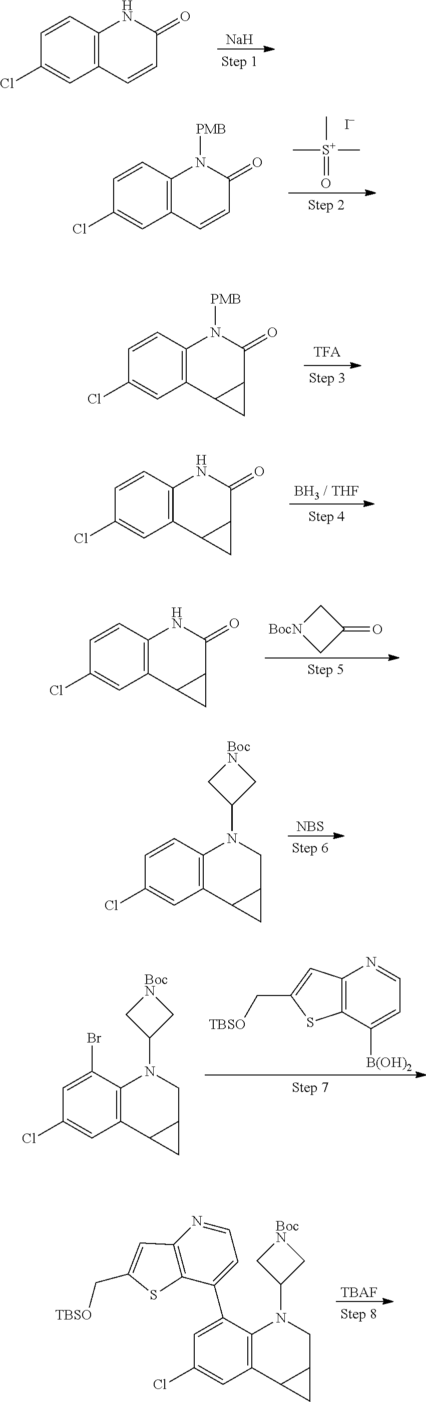

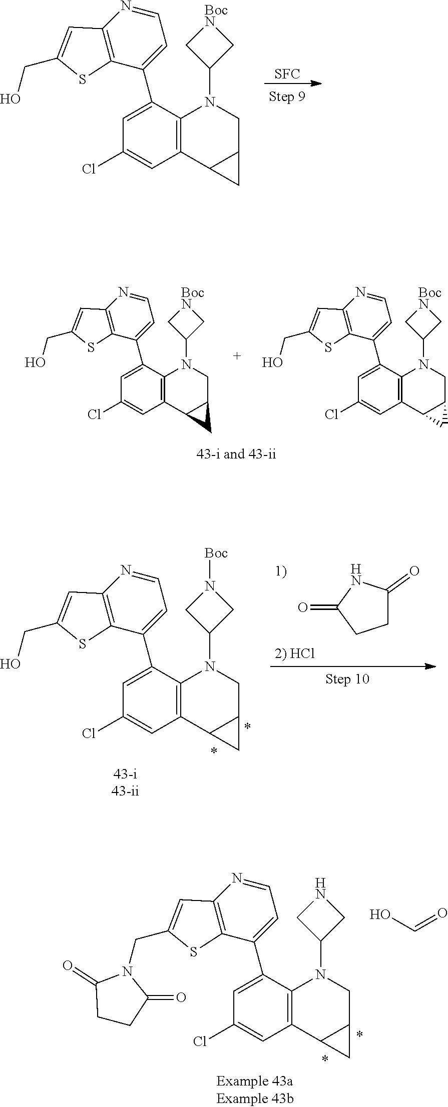

### Step 1: 6-chloro-1-[(4-methoxyphenyl)methyl]quinolin-2-one

To a solution of 6-chloro-1H-quinolin-2-one (2.8 g, 15.59 mmol) in N,N-dimethylformamide (30 mL) were added sodium hydride (748 mg, 18.71 mmol, 60% in mineral oil) and 1-(chloromethyl)-4-methoxybenzene (3.7 g, 23.38 mmol) at 0° C. After the mixture was stirred at 20° C. for 4 h, the reaction mixture was diluted with water (20 mL) and extracted with ethyl acetate (30 mL×3). The combined organic layers were washed with brine (30 mL×3), dried over anhydrous sodium sulfate, filtered and concentrated under reduced pressure. The residue was purified by column chromatography to afford the title compound. LCMS R_{T}=0.980 min, m/z=300.0 [M+H]⁺.

### Step 2: 6-chloro-3-[(4-methoxyphenyl)methyl]-1a,7b-dihydro-1H-cyclopropa[c]quinolin-2-one

To a mixture of sodium hydride (400 mg, 10.01 mmol, 60% in mineral oil) in dimethyl sulfoxide (30 mL) were added 6-chloro-1-[(4-methoxyphenyl)methyl]quinolin-2-one (2.0 g, 6.67 mmol) and trimethylsulfoxonium iodide (2.2 g, 10.01 mmol) at 0° C. After stirring at 45° C. for 12 h under nitrogen atmosphere, the reaction mixture was diluted with water (200 mL) and extracted with ethyl acetate (80 mL×3). The combined organic layers were washed with brine (20×3 mL), dried over anhydrous sodium sulfate, filtered and concentrated under reduced pressure. The residue was purified by column chromatography to afford the title compound. LCMS R_{T}=2.123 min, m/z=314.0 [M+H]⁺.

### Step 3: 6-chloro-1,1a,3,7b-tetrahydrocyclopropa[c]quinolin-2-one

A solution of 6-chloro-3-[(4-methoxyphenyl)methyl]-1a,7b-dihydro-1H-cyclopropa[c]quinolin-2-one (600 mg, 1.91 mmol) in trifluoroacetic acid (3 mL) was stirred at 65° C. for 12 h. The reaction mixture was concentrated under reduced pressure to afford the title compound. It was used directly and without further purification in the next step. LCMS R_{T}=0.840 min, m/z=194.0 [M+H]⁺.

### Step 4: 6-chloro-1a,2,3,7b-tetrahydro-1H-cyclopropa[c]quinoline

A solution of 6-chloro-1,1a,3,7b-tetrahydrocyclopropa[c]quinolin-2-one (900 mg, 4.65 mmol) and borane (1 M in tetrahydrofuran, 14 mL) was stirred at 80° C. for 3 h. The reaction mixture was diluted with methyl alcohol (1 mL). The reaction mixture was concentrated under reduced pressure to afford the title compound. It was used in the next step without further purification. LCMS R_{T}=0.917 min, m/z=180.0 [M+H]⁺.

### Step 5: tert-butyl 3-(6-chloro-1,1a,2,7b-tetrahydrocyclopropa[c]quinolin-3-yl)azetidine-1-carboxylate

A mixture of tert-butyl 3-oxoazetidine-1-carboxylate (1.5 g, 8.57 mmol), 6-chloro-1a,2,3,7b-tetrahydro-1H-cyclopropa[c]quinoline (770 mg, 4.29 mmol), sodium triacetoxyborohydride (2.7 g, 12.86 mmol) and acetic acid (1.3 g, 21.43 mmol) in 1,2-dichloroethane (25 mL) was stirred at 35° C. for 2 h under nitrogen atmosphere. The reaction mixture was diluted with water (20 mL) and extracted with ethyl acetate (20 mL×3). The combined organic layers were washed with brine (15 mL), dried over anhydrous sodium sulfate, filtered and concentrated under reduced pressure. The residue was purified by column chromatography to afford the title compound. LCMS R_{T}=1.163 min, m/z=335.1 [M+H]⁺.

### Step 6: tert-butyl 3-(4-bromo-6-chloro-1,1a,2,7b-tetrahydrocyclopropa[c]quinolin-3-yl)azetidine-1-carboxylate

A mixture of tert-butyl 3-(6-chloro-1,1a,2,7b-tetrahydrocyclopropa[c]quinolin-3-yl)azetidine-1-carboxylate (200 mg, 0.60 mmol) and N-bromosuccinimide (106 mg, 0.60 mmol) in dichloromethane (0.5 mL) and acetic acid (0.5 mL) was stirred at 20° C. for 12 h. The mixture was quenched by addition of saturated aqueous sodium bicarbonate (10 mL) and extracted with ethyl acetate (10 mL×3). The combined organic layers were washed with brine (3×5 mL), dried over anhydrous sodium sulfate, filtered and concentrated under reduced pressure to afford the title compound. LCMS R_{T}=1.255 min, m/z=415.1 [M+H]⁺.

### Step 7: tert-butyl 3-[4-[2-[[tert-butyl(dimethyl)silyl]oxymethyl]thieno[3,2-b]pyridin-7-yl]-6-chloro-1,1a,2,7b-tetrahydrocyclopropa[c]quinolin-3-yl]azetidine-1-carboxylate

To a solution of tert-butyl 3-(4-bromo-6-chloro-1,1a,2,7b-tetrahydrocyclopropa[c]quinolin-3-yl)azetidine-1-carboxylate (70 mg, 0.17 mmol) and [2-[[tert-butyl(dimethyl)silyl]oxymethyl]thieno[3,2-b]pyridin-7-yl]boronic acid (82 mg, 253.79 mmol) in dioxane (5 mL) and water (1 mL) were added cesium carbonate (165 mg, 0.51 mmol) and dichloro[1,1′-bis(diphenylphosphino)ferrocene]palladium(II) (12 mg, 0.017 mmol) at 20° C. After stirring at 100° C. for 12 h, the reaction mixture was diluted with water (30 mL) and extracted with ethyl acetate (20 mL×3). The combined organic layers were washed with brine (60 mL), dried over anhydrous sodium sulfate, filtered and concentrated under reduced pressure. The residue was purified by prep-TLC to afford the title compound. LCMS R_{T}=1.233 min, m/z=612.2 [M+H]⁺

### Step 8: (f)-tert-butyl 3-[6-chloro-4-[2-(hydroxymethyl)thieno[3,2-b]pyridin-7-yl]-1,1a,2,7b-tetrahydrocyclopropa[c]quinolin-3-yl]azetidine-1-carboxylate

To a solution of tert-butyl 3-[4-[2-[[tert-butyl(dimethyl)silyl]oxymethyl]thieno[3,2-b]pyridin-7-yl]-6-chloro-1,1a,2,7b-tetrahydrocyclopropa[c]quinolin-3-yl]azetidine-1-carboxylate (100 mg, 0.16 mmol) in tetrahydrofuran (5 mL) was added tetrabutylammonium fluoride (1 M in tetrahydrofuran, 0.16 mL) at 20° C. After stirring at 20° C. for 1 h, the reaction mixture was concentrated under reduced pressure. The residue was purified by prep-TLC to afford the title compound. LCMS R_{T}=0.947 min, m/z=498.1 [M+H]⁺

### Step 9

The above racemic product (60 mg, 0.12 mmol) was separated by SFC to give first eluting fraction (43-i, Rt=2.862 min; LCMS R_{T}=0.907 min, m/z=498.1 [M+H]+) and second eluting fraction (43-ii, Rt=3.162 min; LCMS R_{T}=0.907 min, m/z=498.1 [M+H]+).

### Step 10

Example 43a was prepared from succinimide (7.96 mg, 0.08 mmol) and 43-i (20 mg, 0.04 mmol) in tetrahydrofuran (1 mL), following the procedure described in the synthesis of Example 1. The final product was purified by RP-HPLC. ¹H NMR (400 MHz, CD₃OD) δ=8.76 (s, 1H), 8.54 (s, 1H), 7.50 (s, 2H), 7.39 (d, J=2.4 Hz, 1H), 7.12 (s, 1H), 4.98 (s, 2H), 4.21-4.10 (m, 1H), 3.98 (s, 1H), 3.85-3.65 (m, 2H), 3.36 (d, J=6.0 Hz, 1H), 3.27 (s, 1H), 2.79 (s, 4H), 2.19-1.80 (m, 3H), 1.11 (dt, J=4.8 Hz, 1H), 0.65 (q, J=4.8 Hz, 1H). LCMS R_{T}=1.003 min, m/z=479.2 [M+H]⁺. (*Absolute stereochemistry not determined.)

Example 43b was prepared from succinimide and 43-ii following the procedure described in the synthesis of Example 1. The final compound was purified by RP-HPLC. ¹H NMR (400 MHz, CD₃OD) δ=8.76 (s, 1H), 8.54 (s, 1H), 7.50 (s, 2H), 7.39 (d, J=2.4 Hz, 1H), 7.12 (s, 1H), 4.98 (s, 2H), 4.21-4.10 (m, 1H), 3.98 (s, 1H), 3.85-3.65 (m, 2H), 3.36 (d, J=6.0 Hz, 1H), 3.27 (s, 1H), 2.79 (s, 4H), 2.19-1.80 (m, 3H), 1.11 (d, J=4.8, Hz, 1H), 0.65 (q, J=4.8 Hz, 1H). LCMS R_{T}=0.998 min, m/z=479.2 [M+H]⁺. (*Absolute stereochemistry not determined.)

### Example 44a, Example 44b, Example 44c and Example 44d: 1-((7-((S)-6-chloro-2-methyl-1-((R)-pyrrolidin-3-yl)-1,2,3,4-tetrahydroquinolin-8-yl)thieno[3,2-b]pyridin-2-yl)methyl)pyrrolidine-2,5-dione, 1-((7-((S)-6-chloro-2-methyl-1-((S)-pyrrolidin-3-yl)-1,2,3,4-tetrahydroquinolin-8-yl)thieno[3,2-b]pyridin-2-yl)methyl)pyrrolidine-2,5-dione, 1-((7-((R)-6-chloro-2-methyl-1-((R)-pyrrolidin-3-yl)-1,2,3,4-tetrahydroquinolin-8-yl)thieno[3,2-b]pyridin-2-yl)methyl)pyrrolidine-2,5-dione and 1-((7-((R)-6-chloro-2-methyl-1-((S)-pyrrolidin-3-yl)-1,2,3,4-tetrahydroquinolin-8-yl)thieno[3,2-b]pyridin-2-yl)methyl)pyrrolidine-2,5-dione

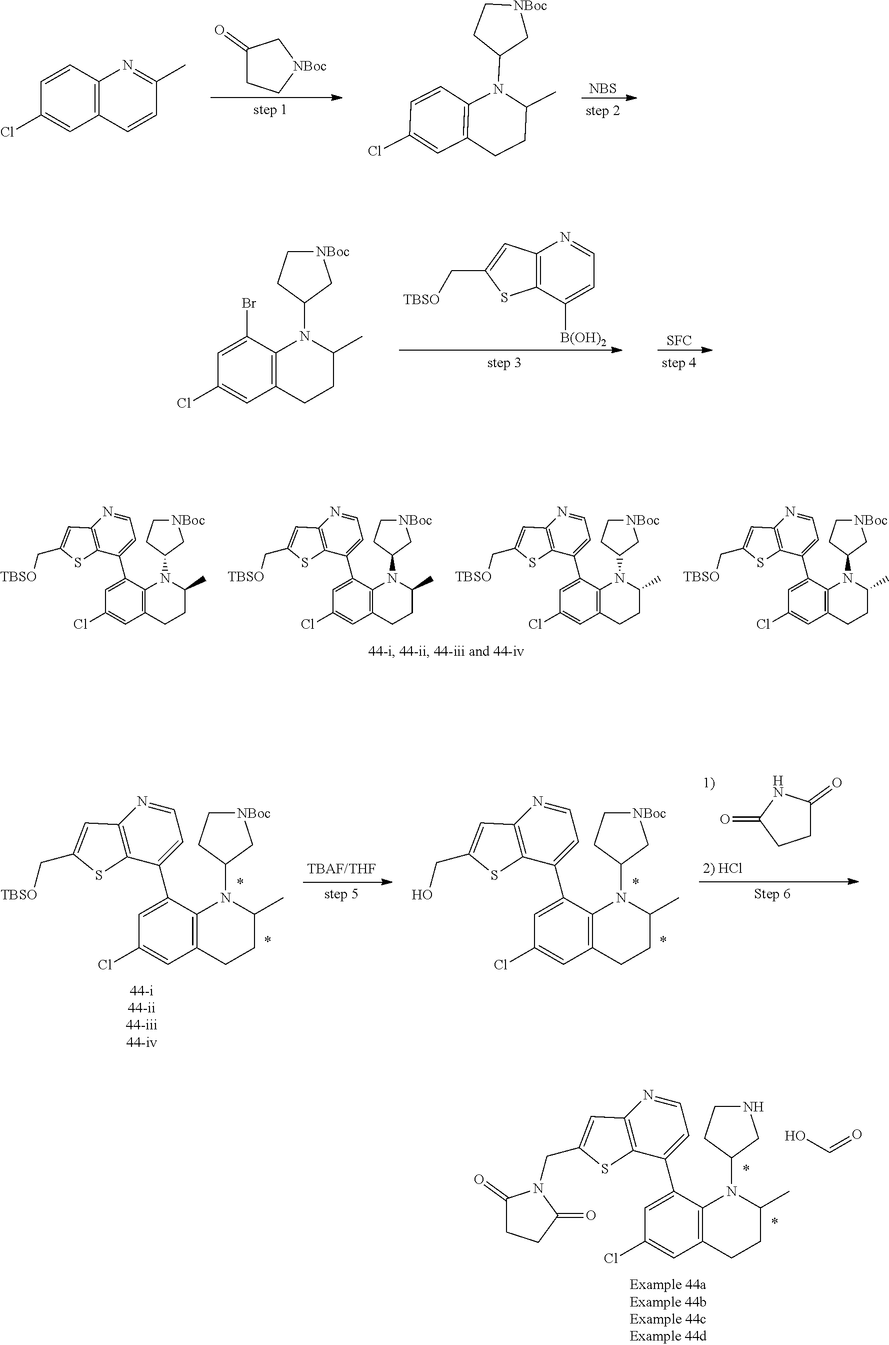

### Step 1: tert-butyl 3-(6-chloro-3,4-dihydroquinolin-1(2H)-yl)azetidine-1-carboxylate

A mixture of 6-chloro-2-methyl-quinoline (2.5 g, 14.07 mmol), tert-butyl 3-oxopyrrolidine-1-carboxylate (3.9 g, 21.11 mmol) and platinum dioxide (64 mg, 0.28 mmol) in ethyl alcohol (20 mL) was stirred under hydrogen atmosphere (50 psi) at 25° C. for 1 hr. The mixture was filtered and the filtrate was concentrated under reduced pressure to afford the title compound. It was used in next step without further purification. LCMS R_{T}=1.129 min, m/z=351.2 [M+H]⁺.

### Step 2: tert-butyl 3-(8-bromo-6-chloro-2-methyl-3,4-dihydroquinolin-1(2H)-yl)pyrrolidine-1-carboxylate

To a solution of tert-butyl 3-(6-chloro-2-methyl-3,4-dihydro-2H-quinolin-1-yl)pyrrolidine-1-carboxylate (1.0 g, 2.86 mmol) was added N-bromosuccinimide (185 mg, 1.04 mmol) at 25° C. After stirring for 1 h at 25° C., the mixture was diluted with water (10 mL) and extracted with ethyl acetate (5 mL×4). The combined organic layers were washed with brine (20 mL), dried over anhydrous sodium sulfate, filtered and concentrated under reduced pressure. The residue was purified by column chromatography to afford the title compound. LCMS R_{T}=1.222 min, m/z=431.0 [M+H]⁺.

### Step 3: (f)-tert-butyl 3-(8-(2-(((tert-butyldimethylsilyl)oxy)methyl)thieno[3,2-b]pyridin-7-yl)-6-chloro-2-methyl-3,4-dihydroquinolin-1(2H)-yl)pyrrolidine-1-carboxylate

To a solution of tert-butyl 3-(8-bromo-6-chloro-2-methyl-3,4-dihydroquinolin-1(2H)-yl)pyrrolidine-1-carboxylate (360 mg, 0.84 mmol) in dioxane (3 mL) and water (0.5 mL) were added potassium carbonate (347 mg, 2.51 mmol) and dichloro[1,1′-bis(diphenylphosphino)ferrocene]palladium(II) at 25° C. After stirring for 1 h at 120° C. under nitrogen atmosphere, the reaction mixture was filtered and concentrated under reduced pressure. The residue was purified by column chromatography to afford the title compound. LCMS R_{T}=1.187 min, m/z=629.9 [M+H]⁺

### Step 4

Tert-butyl 3-(8-(2-(((tert-butyldimethylsilyl)oxy)methyl)thieno[3,2-b]pyridin-7-yl)-6-chloro-2-methyl-3,4-dihydroquinolin-1(2H)-yl)pyrrolidine-1-carboxylate (500 mg, 0.79 mmol) was separated by SFC to give first eluting fraction (44-i, R_{T}=2.641 min), second eluting fraction (44-ii, R_{T}=2.900 min), third eluting fraction (44-iii, R_{T}=3.058 min) and fourth eluting fraction (44-iv, 17%, R_{T}=3.277 min). (*Absolute stereochemistry not determined.)

### Step 5: tert-butyl 3-(-6-chloro-8-(2-(hydroxymethyl)thieno[3,2-b]pyridin-7-yl)-2-methyl-3,4-dihydroquinolin-1(2H)-yl)pyrrolidine-1-carboxylate

To a solution of 44-i (100 mg, 0.15 mmol) in tetrahydrofuran (5 mL) was added a solution of tetrabutylammonium fluoride (1 M in tetrahydrofuran, 0.31 mL) at 25° C. After stirring for 30 min at 25° C., the reaction mixture was concentrated under reduced pressure. The residue was purified by column chromatography to afford the title compound. LCMS R_{T}=0.935 min, m/z=514.1 [M+H]⁺. (*Absolute stereochemistry not determined.)

### Step 6

Example 44a was prepared from succinimide and tert-butyl 3-(-6-chloro-8-(2-(hydroxymethyl)thieno[3,2-b]pyridin-7-yl)-2-methyl-3,4-dihydroquinolin-1(2H)-yl)pyrrolidine-1-carboxylate, following the procedure described in the synthesis of Example 1. The final product was purified by RP-HPLC. ¹H NMR (400 MHz, CD₃OD) δ=8.68 (d, J=4.8 Hz, 1H) 8.53 (s, 1H) 7.51 (s, 1H) 7.41 (d, J=5.2 Hz, 1H) 7.13-7.25 (m, 2H) 4.97 (s, 2H) 3.46-3.67 (m, 2H) 2.77 (s, 5H) 2.70-3.07 (m, 5H) 2.57 (J=8.4 Hz, 1H) 1.68-1.87 (m, 2H) 1.51 (s, 1H) 1.29 (d, J=6.4 Hz, 3H). LCMS R_{T}=0.784 min, m/z=495.1 [M+H]+(*Absolute stereochemistry not determined.)

Example 44b was prepared from 44-ii, following the procedures described in the synthesis of Example 44a. The final compound was purified by RP-HPLC. ¹H NMR (400 MHz, CD₃OD) δ=8.67 (d, J=4.8 Hz, 1H), 8.53 (d, J=1.2 Hz, 1H), 7.50 (s, 1H), 7.39 (d, J=4.8 Hz, 1H), 7.24-7.07 (m, 2H), 4.96 (d, J=2.4 Hz, 2H), 3.64-3.39 (m, 2H), 3.14-3.03 (m, 1H), 2.96-2.86 (m, 2H), 2.75 (s, 4H), 2.64-2.53 (m, 1H), 2.40 (s, 1H), 1.91-1.47 (m, 5H), 1.25 (d, J=6.8 Hz, 3H). LCMS R_{T}=0.804 min, m/z=495.1 [M+H]⁺. (*Absolute stereochemistry not determined.)

Example 44c was prepared from 44-iii, following the procedures described in the synthesis of Example 44a. The final compound was purified by RP-HPLC. ¹H NMR (400 MHz, CD₃OD) δ=8.68 (d, J=4.8 Hz, 1H), 8.54 (d, J=1.6 Hz, 1H), 7.51 (s, 1H), 7.39 (d, J=4.8 Hz, 1H), 7.25-7.07 (m, 2H), 4.97 (d, J=2.4 Hz, 2H), 3.65-3.40 (m, 2H), 3.15-3.04 (m, 1H), 2.97-2.87 (m, 2H), 2.76 (s, 4H), 2.65-2.54 (m, 1H), 2.41 (s, 1H), 1.91-1.48 (m, 5H), 1.26 (d, J=6.8 Hz, 3H). LCMS R_{T}=0.783 min, m/z=495.0 [M+H]⁺. (*Absolute stereochemistry not determined.)

Example 44d was separated from 44-iv, following the procedures described in the synthesis of Example 44a. The final compound was purified by RP-HPLC. ¹H NMR (400 MHz, CD₃OD) δ=8.68 (d, J=4.8 Hz, 1H), 8.54 (d, J=1.6 Hz, 1H), 7.51 (s, 1H), 7.39 (d, J=4.8 Hz, 1H), 7.25-7.07 (m, 2H), 4.97 (d, J=2.4 Hz, 2H), 3.65-3.40 (m, 2H), 3.15-3.04 (m, 1H), 2.97-2.87 (m, 2H), 2.76 (s, 4H), 2.65-2.54 (m, 1H), 2.41 (s, 1H), 1.91-1.48 (m, 5H), 1.26 (d, J=6.8 Hz, 3H). LCMS R_{T}=0.797 min, m/z=495.1 [M+H]⁺. (*Absolute stereochemistry not determined.)

### Example 45: 1-((7-(6-chloro-1-(piperidin-4-yl)-1,2,3,4-tetrahydroquinolin-8-yl)thieno[3,2-b]pyridin-2-yl)methyl)pyrrolidine-2,5-dione, formic acid salt

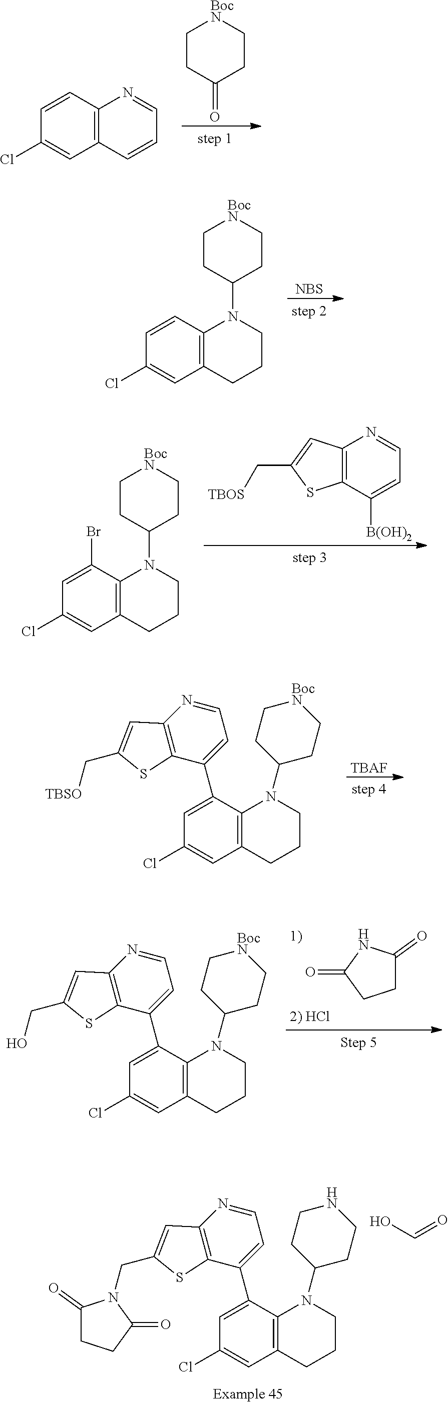

### Step 1: tert-butyl 4-(6-chloro-3,4-dihydroquinolin-1(2H)-yl)piperidine-1-carboxylate

A mixture of 6-chloroquinoline (1.0 g, 6.11 mmol), tert-butyl 4-oxopiperidine-1-carboxylate (1.5 g, 7.33 mmol) and platinum oxide (694 mg, 3.06 mmol) in ethyl alcohol (10 mL) was stirred at 20° C. for 12 h under hydrogen atmosphere (50 psi). The reaction mixture was filtered, and the filtrate was concentrated under reduced pressure. The residue was purified by column chromatography to afford the title compound. LCMS R_{T}=1.123 min, m/z=351.2 [M+H]⁺.

### Step 2: tert-butyl 4-(8-bromo-6-chloro-3,4-dihydroquinolin-1(2H)-yl)piperidine-1-carboxylate

To a solution of tert-butyl 4-(6-chloro-3,4-dihydro-2H-quinolin-1-yl)piperidine-1-carboxylate (180 mg, 0.51 mmol) in N,N-dimethylformamide (5 mL) was added N-bromosuccinimide (91 mg, 0.52 mmol). After stirring at 25° C. for 30 min, the reaction mixture was diluted with water (15 mL) and extracted with ethyl acetate (15 mL×3). The combined organic layers were washed with brine (15 mL), dried over sodium sulfate, filtered and concentrated under reduced pressure to give a residue. The residue was purified by column chromatography afford the title compound. ¹H NMR (400 MHz, CDCl₃) δ=7.36 (s, 1H), 6.93 (s, 1H), 3.70 (t, J=5.8 Hz, 1H), 3.12-3.09 (m, 3H), 2.70-2.67 (m, 4H), 1.79-1.46 (s, 7H), 1.25 (s, 9H).

### Step 3: tert-butyl 4-(8-(2-(((tert-butyldimethylsilyl)oxy)methyl)thieno[3,2-b]pyridin-7-yl)-6-chloro-3,4-dihydroquinolin-1(2H)-yl)piperidine-1-carboxylate

To a solution of tert-butyl 4-(8-bromo-6-chloro-3,4-dihydro-2H-quinolin-1-yl)piperidine-1-carboxylate (200 mg, 465.36 μmol) and [2-[[tert-butyl(dimethyl)silyl]oxymethyl]thieno[3,2-b]pyridin-7-yl]boronic acid (226 mg, 0.70 mmol) in dioxane (10 mL) and water (2 mL) were added dichloro[1,1′-bis(diphenylphosphino)ferrocene]palladium(II) (68 mg, 0.09 mmol) and cesium carbonate (455 mg, 1.40 mmol). After stirring at 140° C. for 30 min under nitrogen, the reaction mixture was quenched by addition of saturated aqueous ammonium chloride solution (15 mL), diluted with water (15 mL) and extracted with ethyl acetate (45 mL). The combined organic layers were washed with brine (15 mL), dried over sodium sulfate, filtered and concentrated under reduced pressure. The residue was purified by column chromatography to afford the title compound. LCMS R_{T}=3.197 min, m/z=628.3 [M+H]⁺.

### Step 4: tert-butyl 4-(6-chloro-8-(2-(hydroxymethyl)thieno[3,2-b]pyridin-7-yl)-3,4-dihydroquinolin-1(2H)-yl)piperidine-1-carboxylate

To a solution of tert-butyl 4-[8-[2-[[tert-butyl(dimethyl)silyl]oxymethyl]thieno[3,2-b]pyridin-7-yl]-6-chloro-3,4-dihydro-2H-quinolin-1-yl]piperidine-1-carboxylate (70 mg, 0.11 mmol) in tetrahydrofuran (2 mL) was added tetrabutylammonium fluoride (1 M in tetrahydrofuran, 0.1 mL). After stirring at 25° C. for 30 min, the reaction mixture was quenched by addition of saturated aqueous ammonium chloride (15 mL), and then diluted with water (15 mL) and extracted with ethyl acetate (15 mL×3). The combined organic layers were washed with brine (15 mL), dried over sodium sulfate, filtered and concentrated under reduced pressure. The residue was purified by column chromatography to afford the title compound. LCMS R_{T}=0.917 min, m/z=514.1 [M+H]⁺.

### Step 5

Example 45 was prepared from tert-butyl 4-[6-chloro-8-[2-(hydroxymethyl)thieno[3,2-b]pyridin-7-yl]-3,4-dihydro-2H-quinolin-1-yl]piperidine-1-carboxylate and succinimide, following the procedure described in the synthesis of Example 1. The final product was purified by RP-HPLC (10-45% acetonitrile in water and 0.05% hydrochloric acid). ¹H NMR (400 MHz, CD₃OD) δ=8.69 (d, J=4.8 Hz, 1H), 8.68 (s, 1H), 7.45-7.44 (m, 2H), 7.16-7.11 (m, 2H), 4.95 (s, 2H), 3.30 (m, 2H), 3.17-3.09 (m, 4H), 2.90-2.82 (m, 3H), 2.75 (s, 4H), 2.16 (s, 2H), 1.92 (d, J=5.4 Hz, 2H), 1.66 (s, 2H). LCMS R_{T}=0.765 min, m/z=495.2 [M+H]⁺.

### Example 46: 1-[[7-[6-chloro-1-(1-methyl-4-piperidyl)-3,4-dihydro-2H-quinolin-8-yl]thieno[3,2-b]pyridin-2-yl]methyl]pyrrolidine-2,5-dione

To a solution of 1-[[7-[6-chloro-1-(4-piperidyl)-3,4-dihydro-2H-quinolin-8-yl]thieno[3,2-b]pyridin-2-yl]methyl]pyrrolidine-2,5-dione (80 mg, 0.16 mmol) in acetonitrile (1 mL) were added formaldehyde (26 mg, 0.32 mmol), sodium triacetoxyborohydride (103 mg, 0.48 mmol) and triethylamine (49 mg, 0.48 mmol) at 25° C. After stirring at 25° C. for 1 h, the reaction mixture was concentrated under reduced pressure. The residue was purified by RP-HPLC to afford the title compound. ¹H NMR (400 MHz, CD₃OD) δ=8.69 (d, J=4.8 Hz, 1H), 8.45 (s, 1H), 7.48-7.40 (m, 2H), 7.17-7.09 (m, 2H), 4.96 (s, 2H), 3.17 (s, 2H), 3.06 (s, 2H), 2.83 (t, J=6.4 Hz, 4H), 2.76 (s, 4H), 2.53 (s, 3H), 2.04 (s, 2H), 1.96-1.87 (m, 2H), 1.70 (s, 2H). LCMS R_{T}=1.001 min, m/z=509.3 [M+H]⁺.

### Example 47a and Example 47b: 1-[[7-[1-[(4R)-azepan-4-yl]-6-chloro-3,4-dihydro-2H-quinolin-8-yl]thieno[3,2-b]pyridin-2-yl]methyl]pyrrolidine-2,5-dione, formic acid salt and 1-[[7-[1-[(4S)-azepan-4-yl]-6-chloro-3,4-dihydro-2H-quinolin-8-yl]thieno[3,2-b]pyridin-2-yl]methyl]pyrrolidine-2,5-dione, formic acid salt

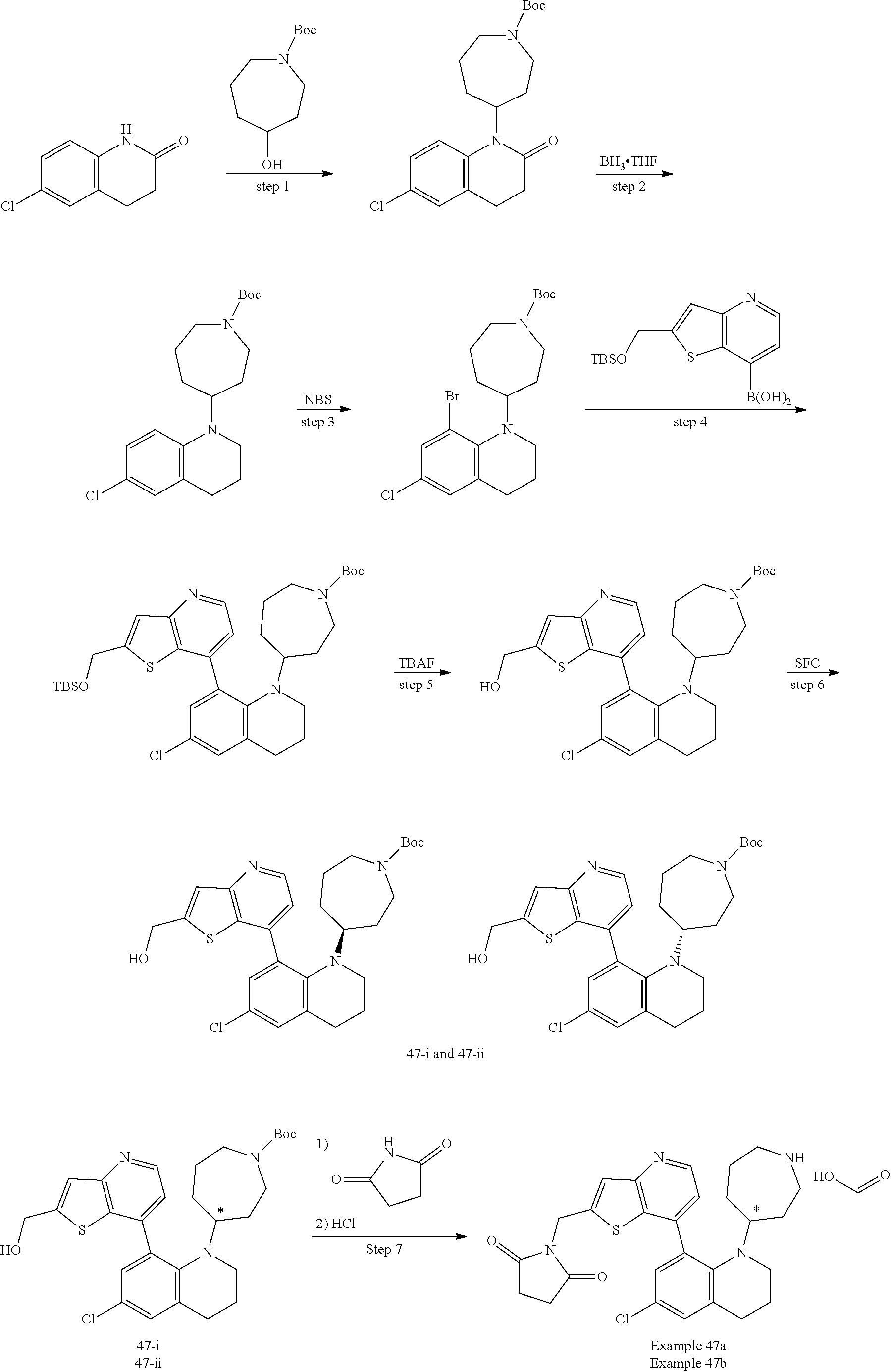

### Step 1: tert-butyl 4-(6-chloro-2-oxo-3,4-dihydroquinolin-1-yl)azepane-1-carboxylate

To a solution of 6-chloro-3,4-dihydro-1H-quinolin-2-one (6.0 g, 33.03 mmol) in toluene (10 mL) were added tert-butyl 4-hydroxyazepane-1-carboxylate (7.1 g, 33.03 mmol) and cyanomethylenetributylphosphorane (12.0 g, 49.56 mmol) at 0° C. After stirring at 110° C. for 8 h, the reaction was concentrated under reduced pressure. The residue was purified by column chromatography to afford the title compound. LCMS R_{T}=1.130 min, m/z=379.2 [M+H]⁺.

### Step 2: tert-butyl 4-(6-chloro-3,4-dihydro-2H-quinolin-1-yl)azepane-1-carboxylate

To a solution of tert-butyl 4-(6-chloro-2-oxo-3,4-dihydroquinolin-1-yl)azepane-1-carboxylate (2.2 g, 3.48 mmol) in tetrahydrofuran (5 mL) was added borane tetrahydrofuran (1 M, 34.84 mmol) at 0° C. After stirring at 20° C. for 5 h, the reaction was quenched by addition of methanol (10 mL). The solvent was removed under reduced pressure and the residue was purified by column chromatography to afford the title compound. LCMS R_{T}=1.153 min, m/z=365.0 [M+H]⁺.

### Step 3: tert-butyl 4-(8-bromo-6-chloro-3,4-dihydro-2H-quinolin-1-yl)azepane-1-carboxylate

To a solution of tert-butyl 4-(6-chloro-3,4-dihydro-2H-quinolin-1-yl)azepane-1-carboxylate (2.0 g, 4.34 mmol) in N,N-dimethylformamide (10 mL) was added N-bromosuccinimide (927 mg, 5.21 mmol) in N,N-dimethylformamide (1 mL) at 0° C. After stirring at 0° C. for 1 h, the reaction mixture was diluted with water (200 mL) and extracted with ethyl acetate (50 mL×4). The combined organic layers were washed with brine (200 mL), dried over anhydrous sodium sulfate, filtered and concentrated under reduced pressure. The residue was purified by column chromatography to afford the title compound. LCMS R_{T}=3.048 min, m/z=445.2 [M+H]⁺.

### Step 4: tert-butyl 4-[8-[2-[[tert-butyl(dimethyl)silyl]oxymethyl]thieno[3,2-b]pyridin-7-yl]-6-chloro-3,4-dihydro-2H-quinolin-1-yl]azepane-1-carboxylate

A mixture of tert-butyl 4-(8-bromo-6-chloro-3,4-dihydro-2H-quinolin-1-yl)azepane-1-carboxylate (1.3 g, 2.93 mmol), [2-[[tert-butyl(dimethyl)silyl]oxymethyl]thieno[3,2-b]pyridin-7-yl]boronic acid (1.4 g, 4.39 mmol), dichloro[1,1′-bis(diphenylphosphino)ferrocene]palladium(II) (643 mg, 0.88 mmol) and potassium carbonate (1.2 g, 8.79 mmol) in water (2 mL) and dioxane (20 mL) under nitrogen atmosphere was stirred at 120° C. in a microwave reactor for 1 h. The reaction mixture was cooled to room temperature and filtered through a pad of Celite and rinsed with ethyl acetate. The filtrate was concentrated under reduced pressure. The residue was purified by column chromatography to afford the title compound. LCMS R_{T}=0.915 min, m/z=642.1 [M+H]⁺

### Step 5: (f)-tert-butyl 4-[6-chloro-8-[2-(hydroxymethyl)thieno[3,2-b]pyridin-7-yl]-3,4-dihydro-2H-quinolin-1-yl]azepane-1-carboxylate

To a solution of tert-butyl 4-[8-[2-[[tert-butyl(dimethyl)silyl]oxymethyl]thieno[3,2-b]pyridin-7-yl]-6-chloro-3,4-dihydro-2H-quinolin-1-yl]azepane-1-carboxylate (2.1 g, 3.27 mmol) in tetrahydrofuran (2 mL) was added tetrabutylammonium fluoride (1 M in tetrahydrofuran, 3.27 mmol) at 20° C. After stirring at 20° C. for 60 min, the reaction was concentrated under reduced pressure. The residue was purified by column chromatography to afford the title compound. LCMS R_{T}=0.923 min, m/z=528.1 [M+H]⁺

### Step 6: tert-butyl (4R)-4-[6-chloro-8-[2-(hydroxymethyl)thieno[3,2-b]pyridin-7-yl]-3,4-dihydro-2H-quinolin-1-yl]azepane-1-carboxylate and tert-butyl (4S)-4-[6-chloro-8-[2-(hydroxymethyl)thieno[3,2-b]pyridin-7-yl]-3,4-dihydro-2H-quinolin-1-yl]azepane-1-carboxylate

The above racemic product (500 mg, 0.95 mmol) was separated by SFC to afford first eluting fraction (47-i, Rt=3.268 min) and second eluting fraction (47-ii, Rₜ=3.510 min). LCMS R_{T}=0.930 min, m/z=528.2 [M+H]⁺. (*Absolute stereochemistry not determined.) Step 7:

To a solution of 47-i (50 mg, 0.095 mmol) and succinimide (19 mg, 0.19 mmol), triphenylphosphine (50 mg, 0.19 mmol) in tetrahydrofuran (1 mL) was added diisopropyl azodicarboxylate (38 mg, 0.19 mmol) at 0° C. After stirring at 0° C. for 10 min, the mixture was heated to 50° C. and stirred for 50 min. The reaction was concentrated under reduced pressure. The residue was dissolved in dioxane (1 mL) and hydrochloric acid (4 M in dioxane, 3 mL) was added. After stirring at 25° C. for 30 min, the reaction mixture was concentrated under reduced pressure. The residue was purified by RP-HPLC to afford Example 47a. ¹H NMR (400 MHz, CD₃OD) δ=8.68 (d, J=4.8 Hz, 1H), 8.52 (s, 1H), 7.45 (s, 1H), 7.42 (d, J=4.8 Hz, 1H), 7.14-7.08 (m, 2H), 4.94 (s, 2H), 3.25-3.09 (m, 2H), 3.03-2.57 (m, 11H), 2.21-1.21 (m, 7H), 0.90 (s, 1H). LCMS R_{T}=0.785 min, m/z=509.0 [M+H]⁺. (*Absolute stereochemistry not determined.)

Example 47b was prepared from 47-ii, following the procedure described in the synthesis of Example 47a. ¹H NMR (400 MHz, CD₃OD) δ=8.68 (d, J=4.8 Hz, 1H), 8.52 (s, 1H), 7.45 (s, 1H), 7.42 (d, J=4.8 Hz, 1H), 7.14-7.08 (m, 2H), 4.94 (s, 2H), 3.25-3.09 (m, 2H), 3.03-2.57 (m, 11H), 2.21-1.21 (m, 7H), 0.90 (s, 1H). LCMS R_{T}=0.554 min, m/z=509.0 [M+H]⁺. (*Absolute stereochemistry not determined.)

### Example 48: 1-((7-(1′-(azetidin-3-yl)-6′-chloro-2′,3′-dihydro-1′H-spiro[cyclopropane-1,4′-quinolin]-8′-yl)thieno[3,2-b]pyridin-2-yl)methyl)pyrrolidine-2,5-dione, formic acid salt

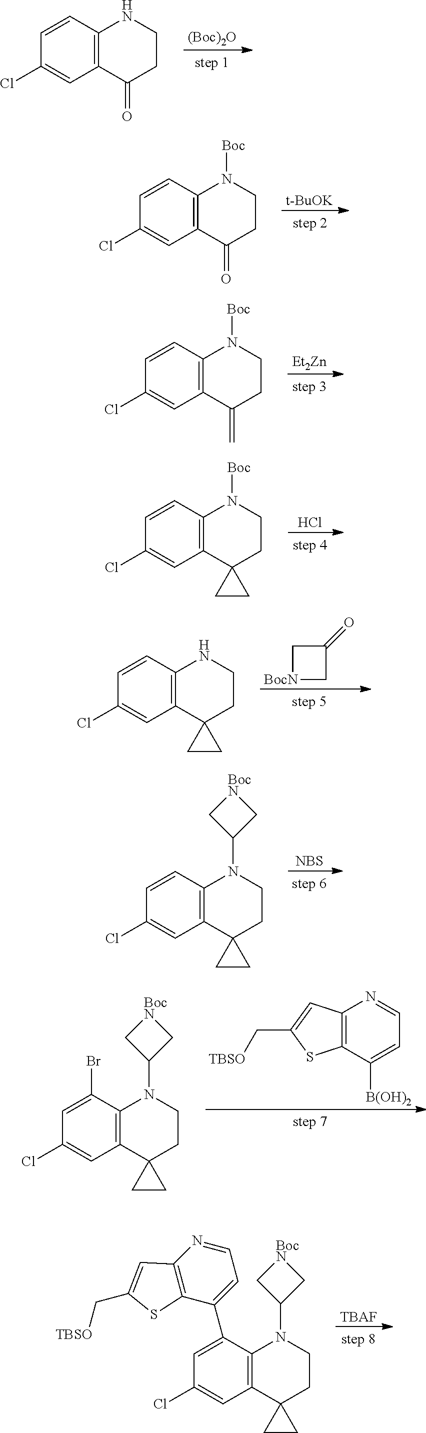

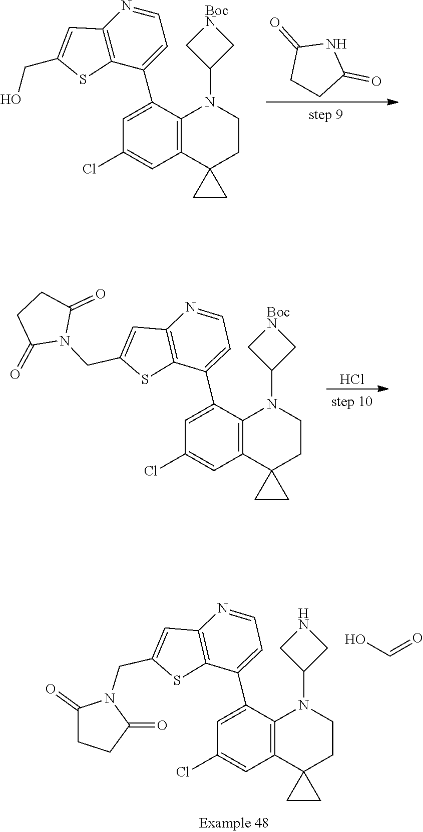

### Step 1: tert-butyl 6-chloro-4-oxo-3, 4-dihydroquinoline-1(2H)-carboxylate

A mixture of 6-chloro-2,3-dihydroquinolin-4(1H)-one (1.4 g, 7.71 mmol), N,N-dimethylpyridin-4-amine (2.8 g, 23.13 mmol) and di-tert-butyl dicarbonate (2.0 g, 9.25 mmol) in dichloromethane (10 mL) was stirred at 40° C. for 2 h. The reaction mixture was washed with water (5 mL×2) and dried over anhydrous sodium sulfate, filtered and concentrated under reduced pressure. The residue was purified by column chromatography to afford the title compound. LCMS R_{T}=1.045 min, m/z=282.2 [M+H]⁺.

### Step 2: tert-butyl 6-chloro-4-methylene-3, 4-dihydroquinoline-1(2H)-carboxylate

A mixture of tert-butyl 6-chloro-4-oxo-3,4-dihydroquinoline-1(2H)-carboxylate (1.0 g, 3.55 mmol), potassium tert-butoxide (597 mg, 5.32 mmol) and methyl(triphenyl)phosphonium bromide (2.5 g, 7.10 mmol) in tetrahydrofuran (30 mL) was stirred at 40° C. for 12 h under nitrogen atmosphere. The reaction mixture was filtered through a pad of Celite and the filtrate was concentrated under reduced pressure. The residue was purified by column chromatography to afford the title compound. LCMS R_{T}=1.421 min, m/z=223.8 [M−56+H]⁺.

### Step 3: tert-butyl 6′-chloro-2′,3′-dihydro-1′H-spiro[cyclopropane-1,4′-quinoline]-1′-carboxylate

To a mixture of tert-butyl 6-chloro-4-methylene-2,3-dihydroquinoline-1-carboxylate (700 mg, 2.50 mmol), diiodomethane (10.1 g, 37.53 mmol) in 1,2-dichloroethane (20 mL) was added diethylzinc (1 M in toluene, 7.51 mL) under nitrogen atmosphere at 0° C. After stirring at 25° C. for 12 h, the reaction mixture was quenched by addition of saturated aqueous ammonium chloride solution (5 mL) and extracted with ethyl acetate (15 mL×3). The combined organic layers were washed with brine (20 mL), dried over anhydrous sodium sulfate, filtered and concentrated under reduced pressure. The residue was purified by column chromatography to afford the title compound. LCMS R_{T}=0.824 min, m/z=193.9 [M−100+H]⁺.

### Step 4: 6′-chloro-2′,3′-dihydro-1′H-spiro[cyclopropane-1,4′-quinoline]

A mixture of tert-butyl 6-chlorospiro[2,3-dihydroquinoline-4,1′-cyclopropane]-1-carboxylate (540 mg, 1.84 mmol) and hydrochloric acid (4 M in dioxane, 10 mL) was stirred at 25° C. for 20 min. The reaction mixture was concentrated under reduced pressure to afford the title compound. LCMS R_{T}=0.826 min, m/z=194.2 [M+H]⁺. It was used in the next step without further purification.

### Step 5: tert-butyl 3-(6′-chloro-2′,3′-dihydro-1′H-spiro[cyclopropane-1,4′-quinolin]-1′-yl)azetidine-1-carboxylate

A mixture of 6′-chloro-2′,3′-dihydro-1′H-spiro[cyclopropane-1,4′-quinoline](340 mg, 1.76 mmol), tert-butyl 3-oxoazetidine-1-carboxylate (450 mg, 2.63 mmol), sodium triacetoxyborohydride (930 mg, 4.39 mmol) in 1,2-dichloroethane (15 mL) and acetic acid (421 mg, 7.02 mmol) was stirred at 30° C. for 12 h under nitrogen atmosphere. The reaction was quenched with saturated aqueous sodium bicarbonate (10 mL), and extracted with ethyl acetate (20 mL×3). The combined organic layers were washed with brine, dried over anhydrous sodium sulfate, filtered and concentrated under reduced pressure. The residue was purified by column chromatography to afford the title compound. LCMS R_{T}=1.408, m/z=348.9 [M+H]⁺.

### Step 6: tert-butyl 3-(8′-bromo-6′-chloro-2′,3′-dihydro-1′H-spiro[cyclopropane-1,4′-quinolin]-1′-yl)azetidine-1-carboxylate

To a solution of tert-butyl 3-(6-chlorospiro[2,3-dihydroquinoline-4,1′-cyclopropane]-1-yl)azetidine-1-carboxylate (255 mg, 0.73 mmol) in chloroform (12 mL) was added N-bromosuccinimide (130 mg, 0.73 mmol) at 0° C. After stirring at 25° C. for 12 h under nitrogen atmosphere, the mixture was washed with brine (0.5 mL), dried over anhydrous sodium sulfate, filtered and concentrated under reduced pressure. The residue was purified by column chromatography to afford the title compound. LCMS R_{T}=1.536 m/z=428.9 [M H]⁺.

### Step 7: tert-butyl 3-(8′-(2-(((tert-butyldimethylsilyl)oxy)methyl)thieno[3,2-b]pyridin-7-yl)-6′-chloro-2′,3′-dihydro-1′H-spiro[cyclopropane-1,4′-quinolin]-1′-yl)azetidine-1-carboxylate

A mixture of tert-butyl 3-(8′-bromo-6′-chloro-2′,3′-dihydro-1′H-spiro[cyclopropane-1,4′-quinolin]-1′-yl)azetidine-1-carboxylate (300 mg, 0.70 mmol), [2-[[tert-butyl(dimethyl)silyl]oxymethyl]thieno[3,2-b]pyridin-7-yl]boronic acid (340 mg, 1.05 mmol), dichloro[1,1′-bis(diphenylphosphino)ferrocene]palladium(II) (103 mg, 0.14 mmol) and cesium carbonate (686 mg, 2.10 mmol) in water (0.8 mL) and dioxane (10 mL) was stirred at 120° C. for 5 h under nitrogen atmosphere. The reaction mixture was cooled to rt and filtered through a pad of Celite. The filtrate was concentrated under reduced pressure. The residue was diluted with water (10 mL) and extracted with ethyl acetate (20 mL×3). The combined organic layers were washed with brine (20 mL), dried over anhydrous sodium sulfate, filtered and concentrated under reduced pressure. The residue was purified by column chromatography to afford the title compound. LCMS R_{T}=1.430, m/z=626.2 [M+H]⁺.

### Step 8: tert-butyl 3-(6′-chloro-8′-(2-(hydroxymethyl)thieno[3,2-b]pyridin-7-yl)-2′,3′-dihydro-1′H-spiro[cyclopropane-1,4′-quinolin]-1′-yl)azetidine-1-carboxylate

To a solution of tert-butyl 3-[8-[2-[[tert-butyl(dimethyl)silyl]oxymethyl]thieno[3,2-b]pyridin-7-yl]-6-chloro-spiro[2,3-dihydroquinoline-4,1′-cyclopropane]-1-yl]azetidine-1-carboxylate (180 mg, 0.29 mmol) in tetrahydrofuran (2 mL) was added tetrabutylammonium fluoride (1 M in tetrahydrofuran, 0.046 mL). After stirring at 25° C. for 20 min, the mixture was concentrated under reduced pressure. The residue was purified by prep-TLC to afford the title compound. LCMS R_{T}=0.957 min, m/z=512.1 [M+H]⁺.

### Step 9: tert-butyl 3-(6′-chloro-8′-(2-((2,5-dioxopyrrolidin-1-yl)methyl)thieno[3,2-b]pyridin-7-yl)-2′,3′-dihydro-1′H-spiro[cyclopropane-1,4′-quinolin]-1′-yl)azetidine-1-carboxylate

To a mixture of tert-butyl 3-(6′-chloro-8′-(2-(hydroxymethyl)thieno[3,2-b]pyridin-7-yl)-2′,3′-dihydro-1′H-spiro[cyclopropane-1,4′-quinolin]-1′-yl)azetidine-1-carboxylate (135 mg, 0.26 mmol), succinimide (78 mg, 0.79 mmol) and triphenylphosphine (207 mg, 0.79 mmol) in tetrahydrofuran (2 mL) was added diisopropylazodicarboxylate (159 mg, 0.79 mmol) under nitrogen atmosphere at 0° C. After stirring at 45° C. for 2 h, the mixture was concentrated under reduced pressure. The residue was purified by prep-TLC to afford the title compound. LCMS R_{T}=1.210 min, m/z=593.1 [M+H]⁺.

### Step 10

A mixture of tert-butyl 3-(6′-chloro-8′-(2-((2,5-dioxopyrrolidin-1-yl)methyl)thieno[3,2-b]pyridin-7-yl)-2′,3′-dihydro-1′H-spiro[cyclopropane-1,4′-quinolin]-l′-yl)azetidine-1-carboxylate (150 mg, 0.25 mmol) in hydrochloric acid (4 M in dioxane, 2 mL) was stirred for 20 min at 20° C. The reaction was concentrated under reduced pressure. The residue was purified by RP-HPLC to afford Example 48. ¹H NMR (400 MHz, CD₃OD) δ=8.73 (d, J=4.8 Hz, 1H), 8.52 (s, 1H), 7.49 (d, J=4.8 Hz, 1H), 7.48-7.47 (m, 1H), 7.15 (d, J=2.4 Hz, 1H), 6.83 (d, J=2.4 Hz, 1H), 4.96 (s, 2H), 4.00 (t, J=8.4 Hz, 1H), 3.56 (s, 2H), 3.33-3.31 (m, 4H), 2.77 (s, 4H), 1.88-1.58 (m, 2H), 1.15 (s, 2H), 1.01 (s, 2H). LCMS R_{T}=0.825 min, m/z=493.1 [M+H]⁺.

### Example 49: 1-[[7-[6-chloro-1-(4-piperidylmethyl)-3,4-dihydro-2H-quinolin-8-yl]thieno[3,2-b]pyridin-2-yl]methyl]pyrrolidine-2,5-dione, hydrochloride acid salt

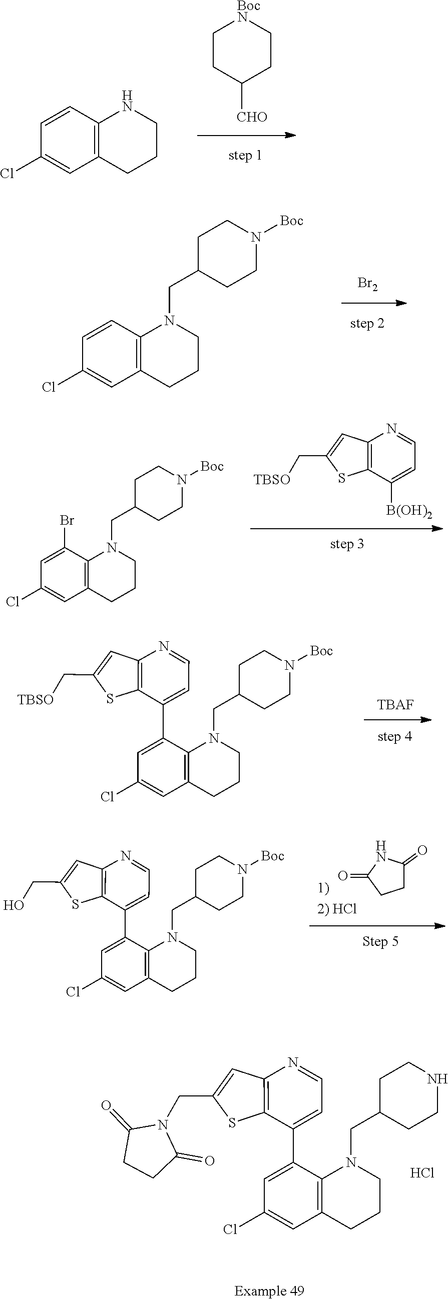

### Step 1: tert-butyl 4-[(6-chloro-3,4-dihydro-2H-quinolin-1-yl)methyl]piperidine-1-carboxylate

To a solution of 6-chloro-1,2,3,4-tetrahydroquinoline (1.0 g, 5.97 mmol) in 1,2-dichloroethane (10 mL) were added sodium triacetoxyborohydride (2.5 g, 11.93 mmol), tert-butyl 4-formylpiperidine-1-carboxylate (1.5 g, 7.16 mmol) and acetic acid (179 mg, 2.98 mmol) at 25° C. After stirring at 25° C. for 12 h, the reaction was quenched by addition of saturated aqueous sodium bicarbonate (40 mL). The mixture was extracted with ethyl acetate (30 mL×3). The combined organic layers were washed with brine (60 mL), dried over sodium sulfate, filtered and concentrated under reduced pressure. The residue was purified by column chromatography to afford the title compound. LCMS R_{T}=1.180 min, m/z=365.0 [M+H]⁺.

### Step 2: tert-butyl 4-[(8-bromo-6-chloro-3,4-dihydro-2H-quinolin-1-yl)methyl]piperidine-1-carboxylate

To a solution of tert-butyl 4-[(6-chloro-3,4-dihydro-2H-quinolin-1-yl)methyl]piperidine-1-carboxylate (1.1 g, 3.01 mmol) in N,N-dimethylformamide (10 mL) were added bromine (963 mg, 6.03 mmol) and iron power (337 mg, 6.03 mmol) at 0° C. After stirring at 25° C. for 1 h, the reaction mixture was quenched by addition of saturated aqueous ammonium chloride (10 mL). The mixture was extracted with dichloroethane (15 mL×3). The combined organic layers were washed with brine (20 mL), dried over drying sodium sulfate, filtered and concentrated under reduced pressure to afford the title compound. LCMS R_{T}=1.227 min, m/z=444.9 [M+H]⁺. The crude product was used in the next step without further purification.

### Step 3: tert-butyl 4-[[8-[2-[[tert-butyl(dimethyl)silyl]oxymethyl]thieno[3,2-b]pyridin-7-yl]-6-chloro-3,4-dihydro-2H-quinolin-1-yl]methyl]piperidine-1-carboxylate

A mixture of tert-butyl 4-[(8-bromo-6-chloro-3,4-dihydro-2H-quinolin-1-yl)methyl]piperidine-1-carboxylate (1.0 g, 2.25 mmol), [2-[[tert-butyl(dimethyl)silyl]oxymethyl]thieno[3,2-b]pyridin-7-yl]boronic acid (1.1 g, 3.38 mmol), dichloro[1,1′-bis(diphenylphosphino)ferrocene]palladium(II) (329 mg, 0.5 mmol) and cesium carbonate (2.2 g, 6.76 mmol) in water (2 mL) and dioxane (10 mL) was stirred at 120° C. for 4 h. The reaction mixture was quenched by addition of saturated aqueous ammonium chloride (15 mL) and extracted with ethyl acetate (15 mL×3). The combined organic layers were washed with brine (15 mL), dried over anhydrous sodium sulfate, filtered and concentrated under reduced pressure. The residue was purified by column chromatography to afford the title compound. LCMS R_{T}=1.170 min, m/z=643.1 [M+H]⁺.

### Step 4: tert-butyl 4-[[6-chloro-8-[2-(hydroxymethyl)thieno[3,2-b]pyridin-7-yl]-3,4-dihydro-2H-quinolin-1-yl]methyl]piperidine-1-carboxylate

To a solution of tert-butyl 4-[[8-[2-[[tert-butyl(dimethyl)silyl]oxymethyl]thieno[3,2-b]pyridin-7-yl]-6-chloro-3,4-dihydro-2H-quinolin-1-yl]methyl]piperidine-1-carboxylate (1.0 g, 1.56 mmol) in tetrahydrofuran (10 mL) was added tetrabutylammonium fluoride (1 M in tetrahydrofuran, 1.56 mL) at 25° C. After stirring at 25° C. for 0.5 h, the reaction mixture was concentrated under reduced pressure. The residue was purified by column chromatography to afford the title compound. LCMS R_{T}=0.924 min, m/z=528.1 [M+H]⁺.

### Step 5

To a solution of succinimide (2 mg, 0.02 mmol), tert-butyl 4-[[6-chloro-8-[2-(hydroxymethyl)thieno[3,2-b]pyridin-7-yl]-3,4-dihydro-2H-quinolin-1-yl]methyl]piperidine-1-carboxylate (20 mg, 0.04 mmol) and triphenylphosphine (10 mg, 0.04 mmol) in tetrahydrofuran (2 mL) was added diisopropyl azodicarboxylate (8 mg, 0.04 mmol) at 0° C. After stirring at 25° C. for 0.5 h, the reaction was concentrated under reduced pressure. To the residue was added hydrochloric acid (4 M in dioxane, 2 mL) and the mixture was stirred for 20 min at 20° C. The reaction was concentrated under reduced pressure. The residue was purified by RP-HPLC to afford Example 49. ¹H NMR (400 MHz, CD₃OD) δ=8.97 (d, J=6.0 Hz, 1H), 8.01 (d, J=6.0 Hz, 1H), 7.74 (s, 1H), 7.30-7.24 (m, 2H), 5.06 (s, 2H), 3.29-3.23 (m, 2H), 3.16 (d, J=10.8 Hz, 2H), 2.90 (t, J=6.0 Hz, 2H), 2.86-2.69 (m, 7H), 2.67 (s, 2H), 2.51-2.35 (m, 2H), 2.02 (s, 1H), 1.73 (d, J=3.6 Hz, 1H), 1.36 (s, 2H), 1.01-0.43 (m, 2H). LCMS R_{T}=0.776 min, m/z=509.1 [M+H]⁺.

### Example 50: 1-[[7-[6-chloro-1-[(1-methyl-4-piperidyl)methyl]-3,4-dihydro-2H-quinolin-8-yl]thieno[3,2-b]pyridin-2-yl]methyl]pyrrolidine-2,5-dione, formic acid salt

To a solution of 1-[[7-[6-chloro-1-(4-piperidylmethyl)-3,4-dihydro-2H-quinolin-8-yl]thieno[3,2-b]pyridin-2-yl]methyl]pyrrolidine-2,5-dione (Example 49, 20 mg, 0.04 mmol) in acetonitrile (3 mL) was added formaldehyde (6 mg, 0.08 mmol), sodium triacetoxyborohydride (25 mg, 0.12 mmol) and triethylamine (12 mg, 0.12 mmol). After stirring at 25° C. for 1 h, the reaction mixture was concentrated under reduced pressure. The final compound was purified by RP-HPLC to afford the title compound. ¹H NMR (400 MHz, CD₃OD) δ=8.67 (d, J=4.0 Hz, 1H), 8.47 (s, 1H), 7.44 (s, 1H), 7.39 (d, J=4.8 Hz, 1H), 7.16 (s, 1H), 7.07 (d, J=2.4 Hz, 1H), 4.94 (s, 2H), 3.17 (s, 4H), 2.86 (t, J=8 Hz, 2H), 2.76 (s, 4H), 2.66 (s, 3H), 2.57 (t, J=12 Hz, 2H), 2.50-2.31 (m, 2H), 1.93 (s, 2H), 1.40-0.62 (m, 4H). LCMS R_{T}=0.780 min, m/z=523.2 [M+H]⁺.

### Example 51a and Example 51b: (S)-1-((7-(6-chloro-1-(piperidin-3-yl)-1,2,3,4-tetrahydroquinolin-8-yl)thieno[3,2-b]pyridin-2-yl)methyl)pyrrolidine-2,5-dione, formic acid salt and (R)-1-((7-(6-chloro-1-(piperidin-3-yl)-1,2,3,4-tetrahydroquinolin-8-yl)thieno[3,2-b]pyridin-2-yl)methyl)pyrrolidine-2,5-dione, formic acid salt

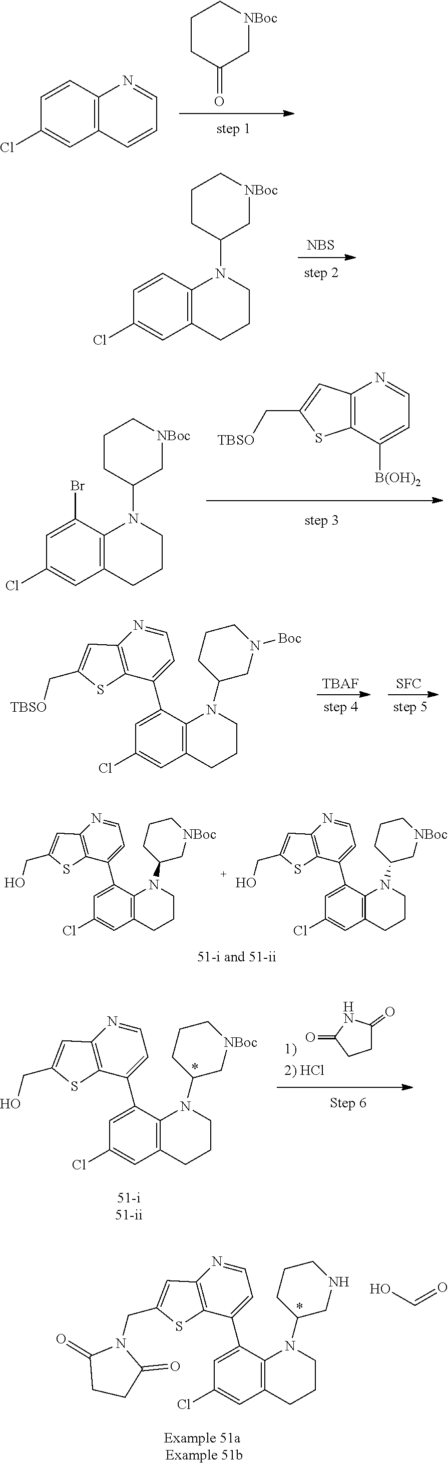

### Step 1: tert-butyl 3-(6-chloro-3,4-dihydroquinolin-1(2H)-yl)piperidine-1-carboxylate

To a solution of 6-chloroquinoline (1.0 g, 6.11 mmol) in ethyl alcohol (10 mL) were added tert-butyl 3-oxopiperidine-1-carboxylate (1.5 g, 7.33 mmol) and platinum oxide (28 mg, 0.12 mmol). The mixture was stirred at 50° C. under hydrogen atmosphere (15 psi) for 12 h. The reaction mixture was filtered and the filtrate was concentrated under reduced pressure. The residue was purified by column chromatography to afford the title compound. LCMS R_{T}=3.007 min, m/z=351.0 [M+H]⁺.

### Step 2: tert-butyl 3-(8-bromo-6-chloro-3,4-dihydroquinolin-1(2H)-yl)piperidine-1-carboxylate

To a solution of tert-butyl 3-(6-chloro-3,4-dihydro-2H-quinolin-1-yl)piperidine-1-carboxylate (380 mg, 1.08 mmol) in N,N-dimethylformamide (1 mL) was added N-bromosuccinimide (193 mg, 1.08 mmol). After the mixture was stirred at 0° C. for 1 h, the reaction mixture was diluted with water (5 mL) and extracted with ethyl acetate (5 mL×3). The combined organic layers were dried over anhydrous sodium sulfate, filtered and concentrated under reduced pressure. The residue was purified by column chromatography to afford the title compound. LCMS R_{T}=3.047 min, m/z=430.9 [M+H]⁺. 1H NMR (400 MHz, CDCl₃) δ=7.37 (s, 1H), 6.95 (s, 1H), 4.23 (d, J=11.6 Hz, 1H), 3.53-3.46 (m, 2H), 3.14 (br, 2H), 2.73-2.70 (m, 3H), 1.51-1.46 (m, 4H), 1.46-1.43 (m, 9H).

### Step 3: tert-butyl 3-(8-(2-(((tert-butyldimethylsilyl)oxy)methyl)thieno[3,2-b]pyridin-7-yl)-6-chloro-3,4-dihydroquinolin-1(2H)-yl)piperidine-1-carboxylate

A mixture of tert-butyl 3-(8-bromo-6-chloro-3,4-dihydro-2H-quinolin-1-yl)piperidine-1-carboxylate (360 mg, 0.84 mmol), [2-[[tert-butyl(dimethyl)silyl]oxymethyl]thieno[3,2-b]pyridin-7-yl]boronic acid (514 mg, 1.59 mmol), dichloro[1,1′-bis(diphenylphosphino)ferrocene]palladium(II) (122 mg, 167.53 μmol) and cesium carbonate (819 mg, 2.51 mmol) in dioxane (10 mL) and water (1 mL) was stirred at 100° C. for 8 h under nitrogen. The reaction mixture was cooled to room temperature, filtered and concentrated under reduced pressure. To the residue was added water (10 mL) and the mixture was extracted with ethyl acetate (10 mL×3). The combined organic layers were washed with brine (5 mL×3), dried over anhydrous sodium sulfate, filtered and concentrated under reduced pressure. The residue was purified by column chromatography to afforded the title compound. LCMS R_{T}=1.197 min, m/z=628.7 [M+H]⁺.

### Step 4: (f)-tert-butyl 3-(6-chloro-8-(2-(hydroxymethyl)thieno[3,2-b]pyridin-7-yl)-3,4-dihydroquinolin-1(2H)-yl)piperidine-1-carboxylate

To a solution of tert-butyl 3-[8-[2-[[tert-butyl(dimethyl)silyl]oxymethyl]thieno[3,2-b]pyridin-7-yl]-6-chloro-3,4-dihydro-2Hquinolin-1-yl]piperidine-1-carboxylate (300 mg, 0.48 mmol) in tetrahydrofuran (5 mL) was added tetrabutylammonium fluoride (1 M in tetrahydrofuran, 1 mL). After stirring at 20° C. for 1 h, the mixture was concentrated under reduced pressure. The residue was purified by prep-TLC to afford the title compound. LCMS R_{T}=0.897 min, m/z=515.9 [M+H]⁺.

### Step 5: tert-butyl (R)-3-(6-chloro-8-(2-(hydroxymethyl)thieno[3,2-b]pyridin-7-yl)-3,4-dihydroquinolin-1(2H)-yl)piperidine-1-carboxylate and tert-butyl (S)-3-(6-chloro-8-(2-(hydroxymethyl)thieno[3,2-b]pyridin-7-yl)-3,4-dihydroquinolin-1(2H)-yl)piperidine-1-carboxylate

The above racemic product (100 mg, 0.19 mmol) was separated by SFC) to give first eluding fraction (53-i, R_{T}=3.023 min) and second eluting fraction (51-ii, R_{T}=2.691 min). LCMS R_{T}=0.893 min, m/z=515.9 [M+H]⁺. (*Absolute stereochemistry not determined.)

### Step 6

Example 51a was prepared from succinimide and 51-i, following the procedure described in the synthesis of Example 1. The final product was purified by RP-HPLC. ¹H NMR (400 MHz, CD₃OD) δ=8.71 (d, J=4.8 Hz, 1H), 8.40 (s, 1H), 7.48 (d, J=4.8 Hz, 1H), 7.45 (s, 1H), 7.16 (dd, J=2.4, 11.2 Hz, 2H), 4.93 (s, 2H), 3.29-3.16 (m, 2H), 2.99 (d, J=10.0 Hz, 2H), 2.91-2.72 (m, 8H), 2.56 (t, J=11.8 Hz, 1H), 2.02-1.81 (m, 2H), 1.66-1.35 (m, 2H), 1.16-0.10 (m, 2H). LCMS R_{T}=0.952 min, m/z=495.1 [M+H]⁺. (*Absolute stereochemistry not determined.)

Example 51b was prepared from succinimide and 51-ii, following the procedure described in the synthesis of Example 1. The final product was purified by RP-HPLC. ¹H NMR (400 MHz, CD₃OD) δ=8.71 (d, J=4.8 Hz, 1H), 8.40 (s, 1H), 7.48 (d, J=4.8 Hz, 1H), 7.45 (s, 1H), 7.16 (dd, J=2.4, 11.2 Hz, 2H), 4.93 (s, 2H), 3.29-3.15 (m, 2H), 2.99 (d, J=10.0 Hz, 2H), 2.91-2.73 (m, 8H), 2.56 (t, J=11.8 Hz, 1H), 2.02-1.81 (m, 2H), 1.66-1.35 (m, 2H), 1.17-0.10 (m, 2H). LCMS R_{T}=0.757 min, m/z=495.1 [M+H]⁺. (*Absolute stereochemistry not determined.)

### Example 52a and 52b: 1-[[7-[(4R)-7-chloro-4-piperazin-1-yl-chroman-5-yl]thieno[3,2-b]pyridin-2-yl]methyl]pyrrolidine-2,5-dione, formic acid salt and 1-[[7-[(4S)-7-chloro-4-piperazin-1-yl-chroman-5-yl]thieno[3,2-b]pyridin-2-yl]methyl]pyrrolidine-2,5-dione, formic acid salt

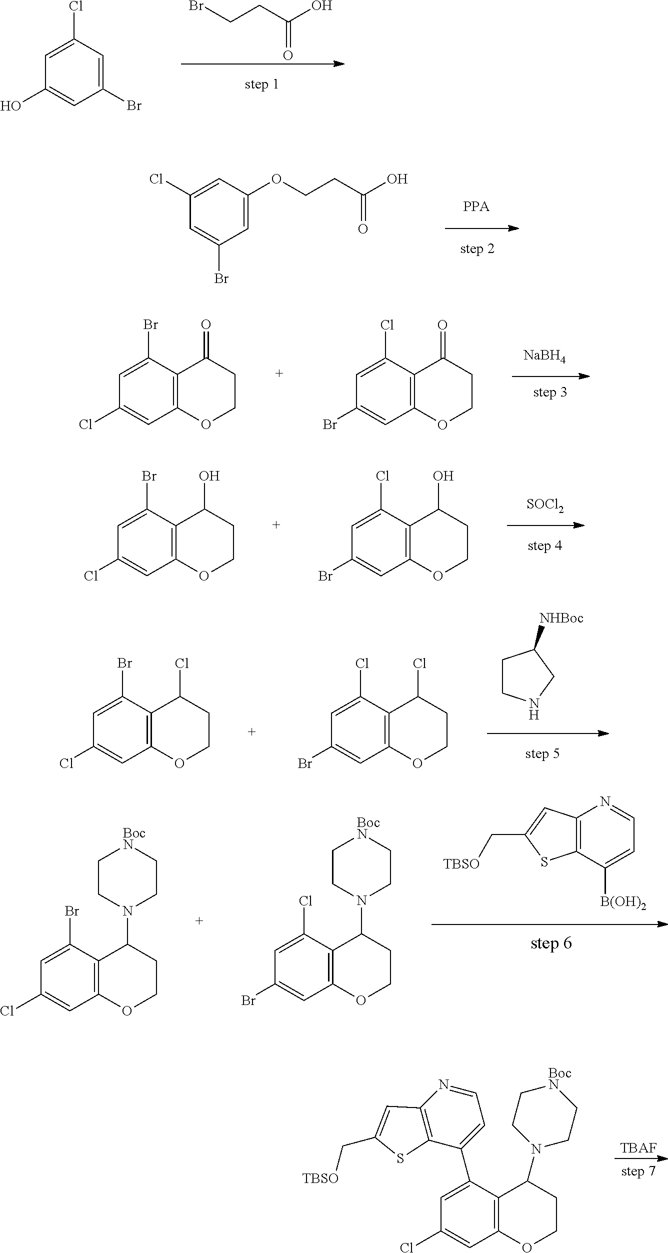

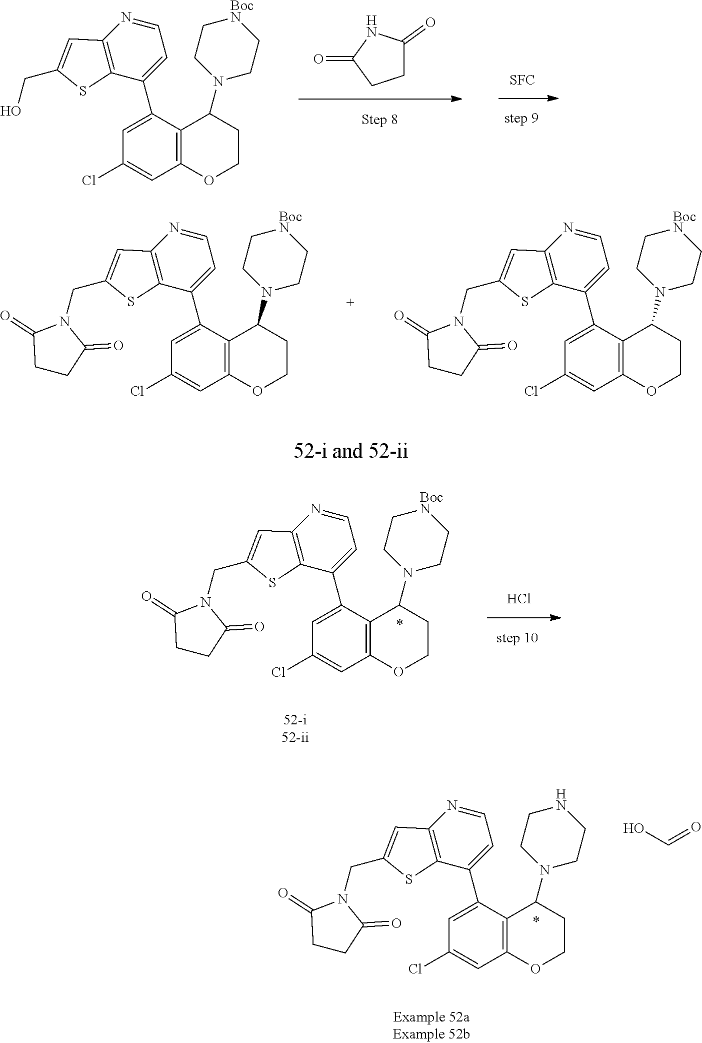

### Step 1: 3-(3-bromo-5-chloro-phenoxy)propanoic acid

To a mixture of 3-bromo-5-chloro-phenol (10.0 g, 48.20 mmol) and 3-bromopropanoic acid (7.4 g, 48.20 mmol) was added a solution of sodium hydroxide (3.9 g, 96.41 mmol) in water (30 mL). After stirring at 100° C. for 16 h, the reaction mixture was quenched by addition of hydrochloric acid (1 M) until pH=4 was reached. The reaction mixture was extracted with ethyl acetate (20 mL×4). The combined organic layers were washed with brine (60 mL), dried over anhydrous sodium sulfate, filtered and concentrated under reduced pressure. The residue was purified by column chromatography to afford the title compound (2.1 g, 16%). ¹H NMR (400 MHz, CD₃OD) δ=7.05 (t, J=1.6 Hz, 1H), 6.90 (t, J=2.0 Hz, 1H), 6.78 (t, J=2.0 Hz, 1H), 4.15 (t, J=6.4 Hz, 2H), 2.78 (t, J=6.4 Hz, 2H)

### Step 2: 5-bromo-7-chloro-chroman-4-one and 7-bromo-5-chloro-chroman-4-on

A mixture of 3-(3-bromo-5-chloro-phenoxy)propanoic acid (2.1 g, 7.51 mmol) in polyphosphoric acid (20 mL) was stirred at 100° C. for 2 h. The reaction was cooled to 40° C. and quenched by addition of methanol (10 mL). The mixture was extracted with ethyl acetate (20 mL×4). The combined organic layers were washed with brine (60 mL), dried over anhydrous sodium sulfate, filtered and concentrated under reduced pressure. The residue was purified by column chromatography to give a mixture of 5-bromo-7-chloro-chroman-4-one and 7-bromo-5-chloro-chroman-4-one.

### Step 3: 5-bromo-7-chloro-chroman-4-ol and 7-bromo-5-chloro-chroman-4-ol

To the product mixture of Step 2 (900 mg, 3.44 mmol) in methanol (20 mL) was added sodium borohydride (1.0 g, 27.53 mmol) at 0° C. After stirring at 20° C. for 1 h, the reaction was quenched by addition of water (30 mL) and extracted with ethyl acetate (10 mL×4). The combined organic layers were washed with brine (30 mL), dried over anhydrous sodium sulfate, filtered and concentrated under reduced pressure to give a mixture of 5-bromo-7-chloro-chroman-4-ol and 7-bromo-5-chloro-chroman-4-ol (830 mg, 92%). It was used in the next step without further purification.

### Step 4: 5-bromo-4,7-dichloro-chromane and 7-bromo-4,5-dichloro-chromane

To a solution of the above mixture (830 mg, 3.15 mmol) in dichloromethane (40 mL) was added thionyl chloride (1.12 g, 9.45 mmol) at 0° C. After stirring at 20° C. for 2 h, the reaction was concentrated under reduced pressure. Saturated aqueous sodium bicarbonate (20 mL) and ethyl acetate (20 mL) were added, and the layers were separated. The aqueous layer was further extracted with ethyl acetate (10 mL×3). The combined organic layers were washed with brine (20 mL), dried over anhydrous sodium sulfate, filtered and concentrated under reduced pressure to give a mixture of 5-bromo-4,7-dichloro-chromane and 7-bromo-4,5-dichloro-chromane.

### Step 5: tert-butyl 4-(5-bromo-7-chloro-chroman-4-yl)piperazine-1-carboxylate and tert-butyl 4-(7-bromo-5-chloro-chroman-4-yl)piperazine-1-carboxylate

To a solution of the above mixture (800 mg, 2.84 mmol) in N,N-dimethylformamide (50 mL) were added tert-butyl piperazine-1-carboxylate (1.1 g, 5.67 mmol), potassium carbonate (3.1 g, 22.70 mmol) and sodium iodide (85 mg, 0.57 mmol) at 20° C. After stirring at 100° C. for 7 h, the reaction was quenched by addition of water (150 mL) and extracted with dichloromethane (25 mL×4). The combined organic layers were washed with brine (75 mL), dried over anhydrous sodium sulfate, filtered and concentrated under reduced pressure. The residue was purified by RP-HPLC to give a mixture of tert-butyl 4-(5-bromo-7-chloro-chroman-4-yl)piperazine-1-carboxylate and tert-butyl 4-(7-bromo-5-chloro-chroman-4-yl)piperazine-1-carboxylate. LCMS R_{T}=2.447 min, m/z=432.9 [M+H]*, LCMS R_{T}=2.520 min, m/z=432.9 [M+H]⁺.

### Step 6: tert-butyl 4-[5-[2-[[tert-butyl(dimethyl)silyl]oxymethyl]thieno[3,2-b]pyridin-7-yl]-7-chloro-chroman-4-yl]piperazine-1-carboxylate and tert-butyl 4-[7-[2-[[tert-butyl(dimethyl)silyl]oxymethyl]thieno[3,2-b]pyridin-7-yl]-5-chloro-chroman-4-yl]piperazine-1-carboxylate

To a solution of the above mixture (330 mg, 0.76 mmol) in water (5 mL) and dioxane (30 mL) were added [2-[[tert-butyl(dimethyl)silyl]oxymethyl]thieno[3,2-b]pyridin-7-yl]boronic acid (371 mg, 1.15 mmol), dichloro[1,1′-bis(diphenylphosphino)ferrocene]palladium(II) (224 mg, 0.31 mmol) and cesium carbonate (996 mg, 3.06 mmol) at 20° C. under nitrogen atmosphere. After stirring at 140° C. for 1 h under microwave, the reaction was filtered and concentrated under reduced pressure. The residue was purified by column chromatography to give tert-butyl 4-[5-[2-[[tert-butyl(dimethyl)silyl]oxymethyl]thieno[3,2-b]pyridin-7-yl]-7-chloro-chroman-4-yl]piperazine-1-carboxylate and tert-butyl 4-[7-[2-[[tert-butyl(dimethyl)silyl]oxymethyl]thieno[3,2-b]pyridin-7-yl]-5-chloro-chroman-4-yl]piperazine-1-carboxylate. LCMS R_{T}=1.350 min, m/z=630.2 [M+H]⁺.

### Step 7: tert-butyl 4-[7-chloro-5-[2-(hydroxymethyl)thieno[3,2-b]pyridin-7-yl]chroman-4-yl]piperazine-1-carboxylate

To a solution of tert-butyl 4-[5-[2-[[tert-butyl(dimethyl)silyl]oxymethyl]thieno[3,2-b]pyridin-7-yl]-7-chloro-chroman-4-yl]piperazine-1-carboxylate (145 mg, 0.23 mmol) in tetrahydrofuran (2 mL) was added a solution of tetrabutylammonium fluoride (1 mL, 1 M in tetrahydrofuran) at 20° C. After stirring for 30 min at 20° C., the reaction mixture was concentrated under reduced pressure. The residue was purified by prep-TLC to afford the title compound. LCMS R_{T}=1.097 min, m/z=516.2 [M+H]⁺.

### Step 8: (f)-tert-butyl 4-[7-chloro-5-[2-[(2,5-dioxopyrrolidin-1-yl)methyl]thieno[3,2-b]pyridin-7-yl]chroman-4-yl]piperazine-1-carboxylate

To a solution of tert-butyl 4-[7-chloro-5-[2-(hydroxymethyl)thieno[3,2-b]pyridin-7-yl]chroman-4-yl]piperazine-1-carboxylate (80 mg, 0.16 mmol) and succinimide (77 mg, 0.78 mmol), triphenylphosphine (203 mg, 0.78 mmol) in tetrahydrofuran (1 mL) was added isopropyl isopropoxycarbonyliminocarbamate (157 mg, 0.78 mmol) at 0° C. After stirring at 0° C. for 60 min, the reaction mixture was diluted with water (15 mL) and extracted with ethyl acetate (10 mL×3). The combined organic layers were washed with brine (15 mL), dried over anhydrous sodium sulfate, filtered and concentrated under reduced pressure. The residue was purified by RP-HPLC to afford the title compound. LCMS R_{T}=2.535 min, m/z=598.7 [M+H]⁺.

### Step 9: tert-butyl 4-[(4S)-7-chloro-5-[2-[(2,5-dioxopyrrolidin-1-yl)methyl]thieno[3,2-b]pyridin-7-yl]chroman-4-yl]piperazine-1-carboxylate and tert-butyl 4-[(4R)-7-chloro-5-[2-[(2,5-dioxopyrrolidin-1-yl)methyl]thieno[3,2-b]pyridin-7-yl]chroman-4-yl]piperazine-1-carboxylate

(±)-tert-butyl 4-[7-chloro-5-[2-[(2,5-dioxopyrrolidin-1-yl)methyl]thieno[3,2-b]pyridin-7-yl]chroman-4-yl]piperazine-1-carboxylate (90 mg, 0.15 mmol) was separated by SFC to afford first eluting fraction (52-i, Rt=4.138 min) and second eluting fraction (52-ii, Rt=4.631 min). (*Absolute stereochemistry not determined.)

### Step 10

To a solution of 52-i (20 mg, 0.03 mmol) in dioxane (1 mL) was added hydrochloric acid (4 M in dioxane, 2 mL) at 20° C. After stirring for 30 min, the reaction mixture was concentrated under reduced pressure. The residue was purified by RP-HPLC to afford Example 52a. ¹H NMR (400 MHz, CD₃OD) δ=8.77-8.60 (m, 1H), 8.57 (s, 1H), 7.59-7.47 (m, 1H), 7.41-7.30 (m, 1H), 7.09-6.79 (m, 2H), 4.97 (s, 2H), 4.22 (s, 3H), 2.85-2.71 (m, 4H), 2.56-1.88 (m, 10H). LCMS R_{T}=1.062 min, m/z=497.3 [M+H]⁺. (*Absolute stereochemistry not determined.)

Example 52b was prepared from 52-ii, following the procedure described in the synthesis of Example 52a. The final product was purified by RP-HPLC. ¹H NMR (400 MHz, CD₃OD) δ=8.76-8.58 (m, 2H), 8.55 (s, 1H), 7.66-7.48 (m, 1H), 7.36 (d, J=4.9 Hz, 1H), 7.09-6.83 (m, 2H), 4.99 (s, 2H), 4.41-4.01 (m, 3H), 2.90-2.68 (m, 4H), 2.61-2.24 (m, 4H), 2.17-1.94 (m, 6H). LCMS R_{T}=1.078 min, m/z=497.4 [M+H]⁺. (*Absolute stereochemistry not determined.)

### Example 53: 1-[[7-[4-(azetidin-3-yl)-7-chloro-3-oxo-1,4-benzoxazin-5-yl]thieno[3,2-b]pyridin-2-yl]methyl]pyrrolidine-2,5-dione

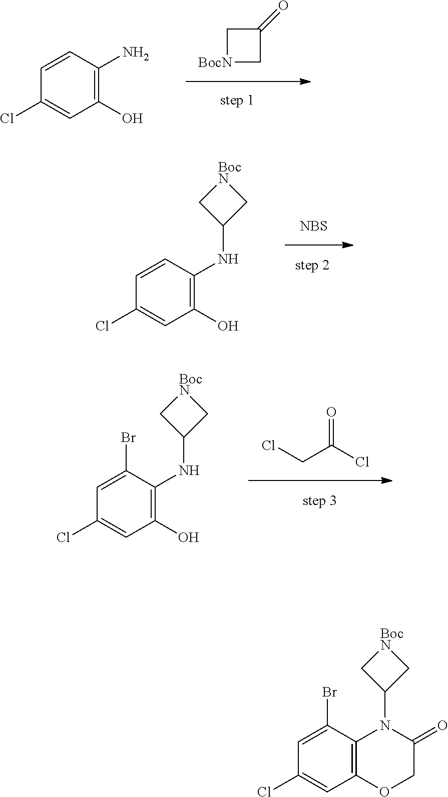

### Step 1: tert-butyl 3-(4-chloro-2-hydroxy-anilino)azetidine-1-carboxylate

To a solution of 2-amino-5-chloro-phenol (3.0 g, 20.90 mmol), tert-butyl 3-oxoazetidine-1-carboxylate (3.6 g, 20.90 mmol) in dichloromethane (30 mL) was added acetic acid (125 mg, 2.09 mmol). The mixture was stirred at 20° C. for 10 min and sodium triacetoxyborohydride (13.29 g, 62.69 mmol) was added. After the mixture was stirred at 20° C. for 12 h, the reaction was quenched by addition of water (20 mL) and extracted with ethyl acetate (30 mL×3). The combined organic layers were washed with brine (30 mL), dried over anhydrous sodium sulfate, filtered and concentrated under reduced pressure. The residue was purified by column chromatography to afford the title compound. LCMS R_{T}=0.954 min, m/z=243.0 [M+H−56]⁺.

### Step 2: tert-butyl 3-(2-bromo-4-chloro-6-hydroxy-anilino)azetidine-1-carboxylate

To a solution of tert-butyl 3-(4-chloro-2-hydroxy-anilino)azetidine-1-carboxylate (2.0 g, 6.69 mmol) in acetonitrile (20 mL) was added N-bromosuccinimide (1.1 g, 6.02 mmol) in tetrahydrofuran (3 mL) dropwise at 0° C. After the mixture was stirred at 0° C. for 1 h, the mixture was concentrated under reduced pressure. The residue was purified by column chromatography to afford the title compound. LCMS R_{T}=0.993 min, m/z=322.9 [M+H−56]⁺.

### Step 3: tert-butyl 3-(5-bromo-7-chloro-3-oxo-1,4-benzoxazin-4-yl)azetidine-1-carboxylate

To a solution of tert-butyl 3-(2-bromo-4-chloro-6-hydroxy-anilino)azetidine-1-carboxylate (140 mg, 0.37 mmol) in acetonitrile (3 mL) was added a solution of potassium carbonate in water (3.75 M, 0.30 mL) and 2-chloroacetyl chloride (63 mg, 0.56 mmol) dropwise at 0° C. After the mixture was stirred at 50° C. for 1 h, the reaction was quenched by addition of water (10 mL) and extracted with ethyl acetate (10 mL×3). The combined organic layers were washed with brine (10 mL), dried over anhydrous sodium sulfate, filtered and concentrated under reduced pressure. The residue was purified by column chromatography to afford the title compound. LCMS R_{T}=1.023 min, m/z=362.9 [M+H−56]⁺.

### Step 4: tert-butyl 3-[5-[2-[[tert-butyl(dimethyl)silyl]oxymethyl]thieno[3,2-b]pyridin-7-yl]-7-chloro-3-oxo-1,4-benzoxazin-4-yl]azetidine-1-carboxylate

To a solution of tert-butyl 3-(5-bromo-7-chloro-3-oxo-1,4-benzoxazin-4-yl)azetidine-1-carboxylate (150 mg, 0.36 mmol), [2-[[tert-butyl(dimethyl)silyl]oxymethyl]thieno[3,2-b]pyridin-7-yl]boronic acid (174 mg, 0.54 mmol) and cesium carbonate (293 mg, 0.90 mmol) in dioxane (3 mL) and water (0.4 mL) was added dichloro[1,1′-bis(diphenylphosphino)ferrocene]palladium(II) (53 mg, 0.07 mmol) under nitrogen atmosphere. After stirring under microwave at 100° C. for 1 h, the reaction was quenched by addition of water (10 mL) and extracted with ethyl acetate (10 mL×3). The combined organic layers were washed with brine (10 mL), dried over anhydrous sodium sulfate, filtered and concentrated under reduced pressure. The residue was purified by column chromatography to afford the title compound. LCMS R_{T}=5.192 min, m/z=616.2 [M+H]⁺.

### Step 5: tert-butyl 3-[7-chloro-5-[2-(hydroxymethyl)thieno[3,2-b]pyridin-7-yl]-3-oxo-1,4-benzoxazin-4-yl]azetidine-1-carboxylate

To a solution of tert-butyl 3-[5-[2-[[tert-butyl(dimethyl)silyl]oxymethyl]thieno[3,2-b]pyridin-7-yl]-7-chloro-3-oxo-1,4-benzoxazin-4-yl]azetidine-1-carboxylate (200 mg, 0.32 mmol) in tetrahydrofuran (3 mL) was added tetrabutylammonium fluoride (1 M in tetrahydrofuran, 0.32 mL). After the mixture was stirred at 20° C. for 0.5 h, the reaction mixture was concentrated under reduced pressure. The residue was purified by column chromatography afford the title compound. LCMS R_{T}=0.879 min, m/z=446.0 [M+H]⁺.

### Step 6

Example 53 was prepared from succinimide and tert-butyl 3-[7-chloro-5-[2-(hydroxymethyl)thieno[3,2-b]pyridin-7-yl]-3-oxo-1,4-benzoxazin-4-yl]azetidine-1-carboxylate, following the procedure described in the synthesis of Example 1. The final product was purified by RP-HPLC. ¹H NMR (400 MHz, CD₃OD) δ=8.83 (d, J=4.8 Hz, 1H), 7.63-7.52 (m, 2H), 7.33 (m, 2H), 4.97 (s, 2H), 4.85-4.80 (m, 1H), 4.76-4.66 (m, 1H), 4.39 (m, 1H), 3.97-3.86 (m, 1H), 3.76-3.63 (m, 2H), 2.85-2.72 (m, 5H). LCMS R_{T}=0.733 min, m/z=483.1 [M+H]⁺.

### Example 54: 1-[[7-[4-(azetidin-3-yl)-7-chloro-2,3-dihydro-1,4-benzoxazin-5-yl]thieno[3,2-b]pyridin-2-yl]methyl]pyrrolidine-2,5-dione, formic acid salt

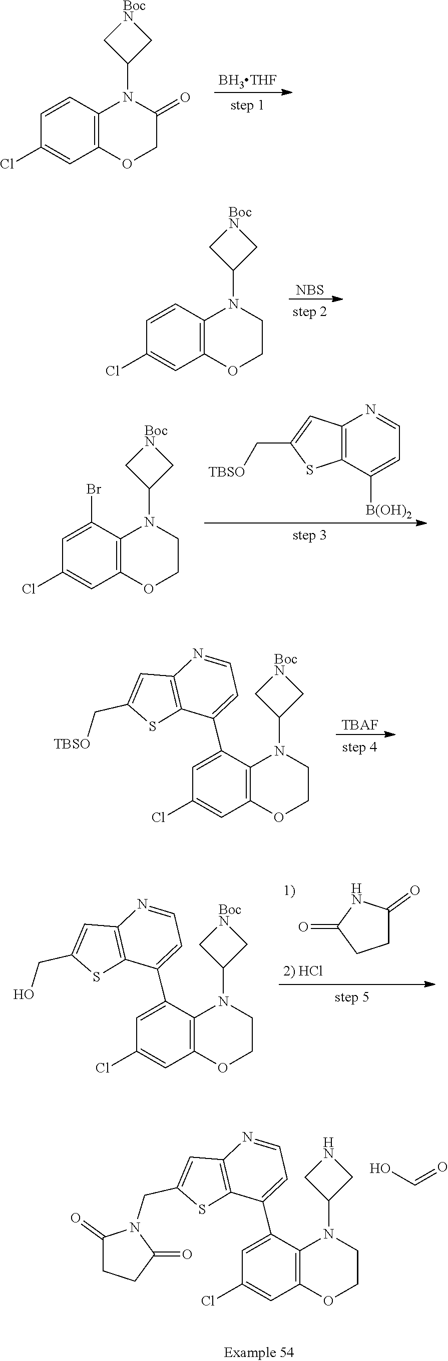

### Step 1: tert-butyl 3-(7-chloro-2,3-dihydro-1,4-benzoxazin-4-yl)azetidine-1-carboxylate

To a solution of tert-butyl 3-(7-chloro-3-oxo-1,4-benzoxazin-4-yl)azetidine-1-carboxylate (1.2 g, 3.42 mmol) in tetrahydrofuran (10 mL) was added borane-tetrahydrofuran (1 M, 10.27 mL) at 0° C. After stirring at 20° C. for 2 h under nitrogen atmosphere, the reaction mixture was diluted with methyl alcohol (2 mL) and aqueous ammonium chloride solution (5 mL) and extracted with ethyl acetate (5 mL×3). The combined organic layers were washed with brine (5 mL×3), dried over sodium sulfate, filtered and concentrated under reduced pressure. The residue was purified by column chromatography to afford the title compound. LCMS R_{T}=1.074 min, m/z=269.0 [M+H−56]⁺.

### Step 2: tert-butyl 3-(5-bromo-7-chloro-2,3-dihydro-1,4-benzoxazin-4-yl)azetidine-1-carboxylate

To a solution of tert-butyl 3-(7-chloro-2,3-dihydro-1,4-benzoxazin-4-yl)azetidine-1-carboxylate (400 mg, 1.23 mmol) in dichloromethane (3 mL) was added N-bromosuccinimide (197 mg, 1.11 mmol) at 25° C. After stirring at 25° C. for 1 h, the reaction mixture was quenched by addition aqueous ammonium chloride solution and extracted with dichloromethane (15 mL×3). The combined organic layers were washed with brine, dried over sodium sulfate, filtered and concentrated under reduced pressure. The residue was purified by column chromatography to afford the title compound. LCMS R_{T}=1.170 min, m/z=348.9 [M+Na−56]⁺.

### Step 3: tert-butyl 3-[5-[2-[[tert-butyl(dimethyl)silyl]oxymethyl]thieno[3,2-b]pyridin-7-yl]-7-chloro-2,3-dihydro-1,4-benzoxazin-4-yl]azetidine-1-carboxylate

To a solution of tert-butyl 3-(5-bromo-7-chloro-2,3-dihydro-1,4-benzoxazin-4-yl)azetidine-1-carboxylate (380 mg, 0.94 mmol) in dioxane (10 mL) were added [2-[[tert-butyl(dimethyl)silyl]oxymethyl]thieno[3,2-b]pyridin-7-yl]boronic acid (456 mg, 1.41 mmol), cesium carbonate (920 mg, 2.82 mmol), water (2 mL) and dichloro[1,1′-bis(diphenylphosphino)ferrocene]palladium(II) (138 mg, 0.19 mmol) at 25° C. After stirring at 140° C. for 1 h, the reaction mixture was quenched by addition of saturated aqueous ammonium chloride solution (15 mL), diluted with water (15 mL) and extracted with ethyl acetate (15 mL×3). The combined organic layers were washed with brine (15 mL), dried over sodium sulfate, filtered and concentrated under reduced pressure. The residue was purified by column chromatography to afford the title compound. LCMS R_{T}=1.107 min, m/z=602.2 [M+H]⁺.

### Step 4: tert-butyl 3-[7-chloro-5-[2-(hydroxymethyl)thieno[3,2-b]pyridin-7-yl]-2,3-dihydro-1,4-benzoxazin-4-yl]azetidine-1-carboxylate

To a solution of tert-butyl 3-[5-[2-[[tert-butyl(dimethyl)silyl]oxymethyl]thieno[3,2-b]pyridin-7-yl]-7-chloro-2,3-dihydro-1,4-benzoxazin-4-yl]azetidine-1-carboxylate (380 mg, 0.63 mmol) in tetrahydrofuran (10 mL) was added tetrabutylammonium fluoride (1 M in tetrahydrofuran, 0.7 mL) at 25° C. After stirring at 25° C. for 0.5 h, the reaction mixture was concentrated under reduced pressure. The residue was purified by column chromatography to afford the title compound. LCMS R_{T}=0.865 min, m/z=488.1 [M+H]⁺.

### Step 5

To a solution of tert-butyl 3-[7-chloro-5-[2-(hydroxymethyl)thieno[3,2-b]pyridin-7-yl]-2,3-dihydro-1,4-benzoxazin-4-yl]azetidine-1-carboxylate (180 mg, 0.37 mmol), succinimide (73 mg, 0.73 mmol) and triphenylphosphine (194 mg, 0.74 mmol) in tetrahydrofuran (2 mL) was added diisopropyl azodicarboxylate (149 mg, 0.74 mmol) at 0° C. After stirring at 50° C. for 1 h, the reaction was concentrated under reduced pressure. To the residue was added hydrochloric acid (4 M in dioxane, 2 mL) and the mixture was stirred for 20 min at 20° C. The reaction was concentrated under reduced pressure. The residue was purified by RP-HPLC to afford Example 54. ¹H NMR (400 MHz, CD₃OD) δ=8.71 (s, 1H), 8.49 (s, 1H), 7.51-7.43 (m, 2H), 7.13-6.71 (m 2H), 4.95 (s, 2H), 4.19 (s, 2H), 3.93 (s, 2H), 3.72 (t, J=6.4 Hz, 1H), 3.57 (s, 2H), 3.31 (s, 2H), 2.77 (s, 4H). LCMS R_{T}=0.718 min, m/z=469.0 [M+H]⁺.

### Example 55: (S)-3-((7-(6-chloro-1-(pyrrolidin-3-yl)-1,2,3,4-tetrahydroquinolin-8-yl)thieno[3,2-b]pyridin-2-yl)methyl)-1-(2,2,2- trifluoroethyl)pyrimidine-2,4(1H,3H)-dione, hydrochloride acid salt

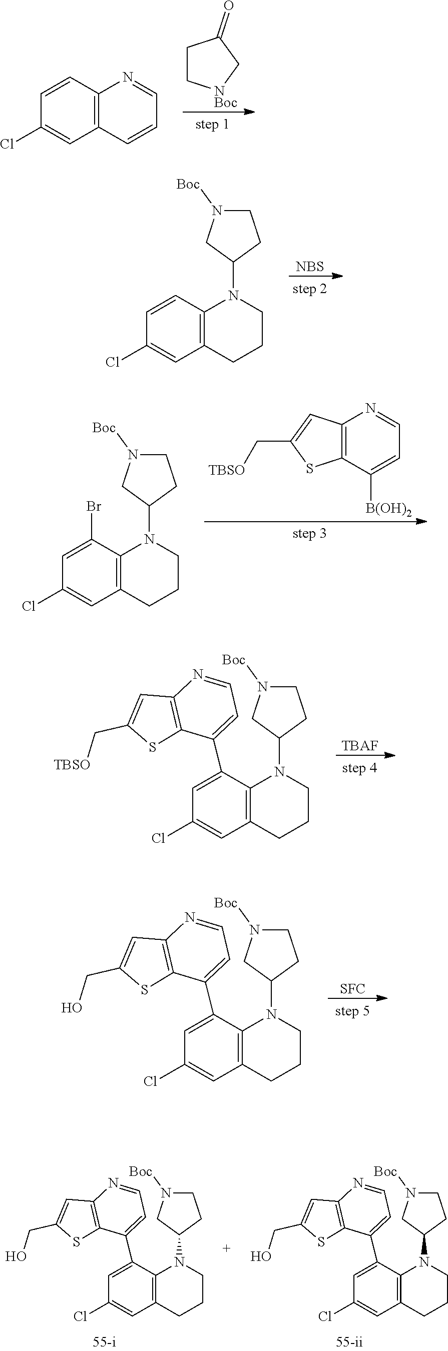

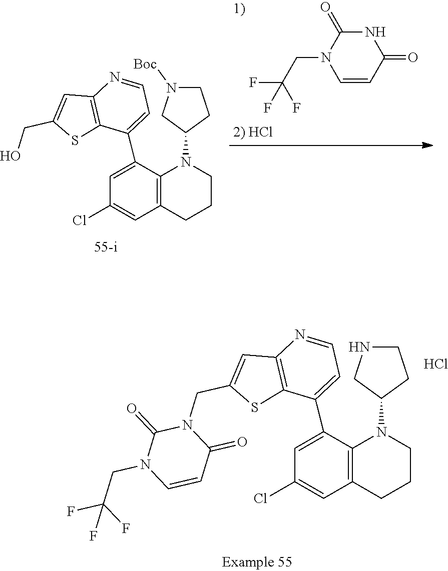 [SMILES: CC(C)(C)OC(=O)N1CC[C@H](N2CCCc3cc(Cl)cc(-c4ccnc5cc(CO)sc45)c32)C1 ; O=c1ccn(CC(F)(F)F)c(=O)[nH]1 ; O=c1ccn(CC(F)(F)F)c(=O)n1Cc1cc2nccc(-c3cc(Cl)cc4c3N([C@H]3CCNC3)CCC4)c2s1 ; Cl]

### Step 1: tert-butyl 3-(6-chloro-3,4-dihydroquinolin-1(2H)-yl)pyrrolidine-1-carboxylate

To a solution of tert-butyl 3-oxopyrrolidine-1-carboxylate (8.5 g, 45.84 mmol) and 6-chloroquinoline (5.0 g, 30.56 mmol) in ethanol (50 mL) was added platinum dioxide (485 mg, 2.14 mmol) and the mixture was stirred at 50° C. for 12 h under hydrogen atmosphere (15 psi). The reaction mixture was filtered and the filtrate was concentrated under reduced pressure. The residue was purified by column chromatography to afford the title compound. LCMS R_{T}=1.436 min, m/z=337.2 [M+H]⁺.

### Step 2: tert-butyl 3-(8-bromo-6-chloro-3,4-dihydroquinolin-1(2H)-yl)pyrrolidine-1-carboxylate

To a solution of tert-butyl 3-(6-chloro-3,4-dihydroquinolin-1(2H)-yl)pyrrolidine-1-carboxylate (7.1 g, 21.02 mmol) in dichloromethane (70 mL) was added N-bromosuccinimide (3.7 g, 21.02 mmol). After stirring at 25° C. for 1 h, the mixture was concentrated under reduced pressure. The residue was purified by column chromatography to afford the title compound LCMS R_{T}=1.245 min, m/z=417.4 [M+H]⁺.

### Step 3: tert-butyl 3-(8-(2-(((tert-butyldimethylsilyl)oxy)methyl)thieno[3,2-b]pyridin-7-yl)-6-chloro-3,4-dihydroquinolin-1(2H)-yl)pyrrolidine-1-carboxylate

To a solution of (2-(((tert-butyldimethylsilyl)oxy)methyl)thieno[3,2-b]pyridin-7-yl)boronic acid (1.0 g, 3.09 mmol), tert-butyl 3-(8-bromo-6-chloro-3,4-dihydroquinolin-1(2H)-yl)pyrrolidine-1-carboxylate (1.0 g, 2.41 mmol) and cesium carbonate (998 mg, 3.06 mmol) in dioxane (30 mL) and water (5 mL) was added dichloro[1,1′-bis(diphenylphosphino)ferrocene]palladium(II) (351 mg, 0.49 mmol). After stirring at 120° C. for 4 h under nitrogen atmosphere, the reaction mixture was filtered and the filtrate was concentrated under reduced pressure. The residue was purified by column chromatography to afford the title compound. LCMS R_{T}=2.815 min, m/z=614.3 [M+H]⁺.

### Step 4: (f)-tert-butyl 3-(6-chloro-8-(2-(hydroxymethyl)thieno[3,2-b]pyridin-7-yl)-3,4-dihydroquinolin-1(2H)-yl)pyrrolidine-1-carboxylate

To a solution of tert-butyl 3-(8-(2-(((tert-butyldimethylsilyl)oxy)methyl)thieno[3,2-b]pyridin-7-yl)-6-chloro-3,4-dihydroquinolin-1(2H)-yl)pyrrolidine-1-carboxylate (1.2 g, 1.95 mmol) in tetrahydrofuran (10 mL) was added tetrabutylammonium fluoride (1 M in tetrahydrofuran, 1.95 mL). After stirring at 25° C. for 0.5 h, the reaction mixture was concentrated under reduced pressure. The residue was purified by column chromatography to afford the title compound. LCMS R_{T}=0.881 min, m/z=500.2 [M+H]⁺.

### Step 5: (S)-tert-butyl 3-(6-chloro-8-(2-(hydroxymethyl)thieno[3,2-b]pyridin-7-yl)-3,4-dihydroquinolin-1(2H)-yl)pyrrolidine-1-carboxylate and (R)-tert-butyl 3-(6-chloro-8-(2-(hydroxymethyl)thieno[3,2-b]pyridin-7-yl)-3,4-dihydroquinolin-1(2H)-yl)pyrrolidine-1-carboxylate

The racemic product from Step 4 (0.45 g, 0.9 mmol) was separated by SFC to give first eluting fraction (55-i, 210 mg, Rt=2.335 min) and second eluting fraction (55-ii, 210 mg, Rₜ=3.054 min). LCMS R_{T}=0.881 min, m/z=500.2 [M+H]⁺.

### Step 6

Example 55 was prepared from 55-i and 1-(2,2,2-trifluoroethyl)pyrimidine-2,4(1H,3H)-dione, following the procedure described in the synthesis of Example 1. The final compound was purified by RP-HPLC. ¹H NMR (400 MHz, CD₃OD) δ=8.72 (d, J=4.8 Hz, 1H), 8.51 (s, 1H), 7.67 (d, J=8.0 Hz, 1H), 7.57-7.43 (m, 2H), 7.25-7.13 (m, 2H), 5.89 (d, J=8.0 Hz, 1H), 5.39-5.28 (m, 2H), 4.71-4.55 (m, 2H), 3.70 (t, J=8.4 Hz, 1H), 3.30-3.07 (m, 3H), 2.96-2.45 (m, 4H), 2.06-1.42 (m, 4H). LCMS R_{T}=0.801 min, m/z=576.0 [M+H]⁺. The absolute configuration of Example 55 was determined by vibrational circular dichroism (VCD).

### Example 56: (R)-3-((7-(6-chloro-1-(pyrrolidin-3-yl)-1,2,3,4-tetrahydroquinolin-8-yl)thieno[3,2-b]pyridin-2-yl)methyl)-1-(2,2,2-trifluoroethyl)pyrimidine-2,4(1H,3H)-dione, formic acid salt

Example 56 was prepared from 55-ii and 1-(2,2,2-trifluoroethyl)pyrimidine-2,4(1H,3H)-dione, following the procedure described in the synthesis of Example 1. The final compound was purified by RP-HPLC. ¹H NMR (400 MHz, CD₃OD) δ=8.78 (d, J=5.2 Hz, 1H), 8.51 (s, 1H), 7.76-7.55 (m, 3H), 7.23 (q, J=2.4 Hz, 2H), 5.90 (d, J=8.0 Hz, 1H), 5.43 (s, 2H), 4.72-4.57 (m, 2H), 3.70 (q, J=8.4 Hz, 1H), 3.32-3.13 (m, 4H), 2.99-2.53 (m, 4H), 2.14-1.49 (m, 4H). LCMS R_{T}=0.803 min, m/z=576.0 [M+H]⁺. The absolute configuration of Example 56 was determined by vibrational circular dichroism (VCD).

### Example 57: (S)-1-((7-(6-chloro-1-(pyrrolidin-3-yl)-1,2,3,4-tetrahydroquinolin-8-yl)thieno[3,2-b]pyridin-2-yl)methyl)pyrrolidine-2,5-dione, formic acid salt

Example 57 was prepared from 55-ii and succinimide, following the procedure described in the synthesis of Example 1. The final product was purified by RP-HPLC. ¹H NMR (400 MHz, CD₃OD) δ=8.72 (d, J=4.8 Hz, 1H), 8.52 (s, 1H), 7.53-7.43 (m, 2H), 7.17 (dd, J=2.4, 10.0 Hz, 2H), 4.95 (s, 2H), 3.68 (q, J=8.4 Hz, 1H), 3.27-3.02 (m, 4H), 2.89 (t, J=6.4 Hz, 2H), 2.76 (s, 6H), 2.01-1.58 (m, 4H). LCMS R_{T}=1.036 min, m/z=481.1 [M+H]⁺.

### Example 58: (R)-1-((7-(6-chloro-1-(pyrrolidin-3-yl)-1,2,3,4-tetrahydroquinolin-8-yl)thieno[3,2-b]pyridin-2-yl)methyl)pyrrolidine-2,5-dione, formic acid salt

Example 58 was prepared from 55-ii and succinimide, following the procedure described in the synthesis of Example 1. The final product was purified by RP-HPLC. ¹H NMR (400 MHz, CD₃OD) δ=8.73 (d, J=4.8 Hz, 1H), 8.51 (s, 1H), 7.55-7.43 (m, 2H), 7.19 (dd, J=2.4, 11.6 Hz, 2H), 4.96 (s, 2H), 3.70 (q, J=8.4 Hz, 1H), 3.29-3.11 (m, 4H), 2.91 (t, J=6.4 Hz, 2H), 2.77 (s, 6H), 2.03-1.62 (m, 4H). LCMS R_{T}=1.043 min, m/z=481.1 [M+H]⁺.

### Example 59: (S)-1-((7-(6-chloro-1-(1-methylpyrrolidin-3-yl)-1,2,3,4-tetrahydroquinolin-8-yl)thieno[3,2-b]pyridin-2-yl)methyl)pyrrolidine-2,5-dione, formic acid salt

To a solution of (S)-1-((7-(6-chloro-1-(pyrrolidin-3-yl)-1,2,3,4-tetrahydroquinolin-8-yl)thieno[3,2-b]pyridin-2-yl)methyl)pyrrolidine-2,5-dione (Example 57, 30 mg, 0.06 mmol) in acetonitrile (1 mL) were added formaldehyde (10 mg, 0.12 mmol), sodium triacetoxyborohydride (39 mg, 0.19 mmol) and triethylamine (19 mg, 0.19 mmol). After stirring at 25° C. for 1 h, the reaction was concentrated under reduced pressure. The residue was purified by RP-HPLC to afford the title compound. ¹H NMR (400 MHz, CD₃OD) δ=8.72 (d, J=4.8 Hz, 1H), 8.45 (s, 1H), 7.49-7.46 (m, 2H), 7.18-7.15 (m, 2H), 4.95 (s, 2H), 3.75 (m, J=8.4 Hz, 1H), 3.26 (s, 2H), 2.95-2.81 (m, 4H), 2.76 (s, 6H), 2.4 (s, 3H), 2.05-1.88 (m, 4H). LCMS RT=0.701 min, m/z=495.3 [M+H]⁺.

### Example 60: (R)-1-((7-(6-chloro-1-(1-methylpyrrolidin-3-yl)-1,2,3,4-tetrahydroquinolin-8-yl)thieno[3,2-b]pyridin-2-yl)methyl)pyrrolidine-2,5-dione, formic acid salt

Example 60 was prepared from (R)-1-((7-(6-chloro-1-(pyrrolidin-3-yl)-1,2,3,4-tetrahydroquinolin-8-yl)thieno[3,2-b]pyridin-2-yl)methyl)pyrrolidine-2,5-dione (Example 58), following the procedure described in the synthesis of Example 59. The residue was purified by RP-HPLC. ¹H NMR (400 MHz, CD₃OD) δ=8.70 (d, J=4.8 Hz, 1H), 8.47 (s, 1H), 7.52-7.42 (m, 2H), 7.18-7.08 (m, 2H), 4.94 (s, 2H), 3.74 (q, J=8.4 Hz, 1H), 3.30-3.14 (m, 2H), 3.14-2.80 (m, 4H), 2.75 (s, 6H), 2.59-2.33 (m, 3H), 2.04-1.45 (m, 4H). LCMS R_{T}=1.530 min, m/z=495.2 [M+H]⁺.

### Example 61: (S)-3-((7-(6-chloro-1-(pyrrolidin-3-yl)-1,2,3,4-tetrahydroquinolin-8-yl)thieno[3,2-b]pyridin-2-yl)methyl)-1-(2-(difluoromethoxy)ethyl)pyrimidine-2,4(1H,3H)-dione, formic acid salt

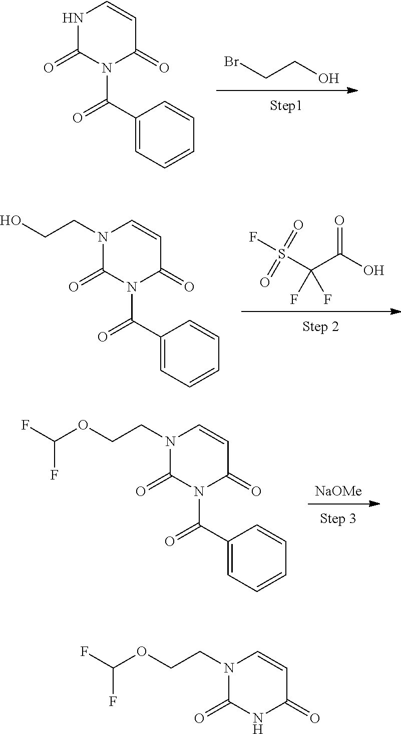

### Step 1: 3-benzoyl-1-(2-hydroxyethyl)pyrimidine-2,4(1H,3H)-dione

To a solution of 3-benzoyl-1H-pyrimidine-2,4-dione (600 mg, 2.78 mmol) in N,N-dimethylformamide (5 mL) were added 2-bromoethanol (693 mg, 5.55 mmol) and potassium carbonate (767 mg, 5.55 mmol) at 25° C. After stirring at 50° C. for 2 h, the reaction was filtered and concentrated under reduced pressure. The residue was purified by column chromatography to afford the title compound. LCMS R_{T}=0.740 min, m/z=261.0 [M+H]⁺.

### Step 2: 3-benzoyl-1-(2-(difluoromethoxy)ethyl)pyrimidine-2,4(1H,3H)-dione

To a solution of 3-benzoyl-1-(2-hydroxyethyl)pyrimidine-2,4(1H,3H)-dione (490 mg, 1.88 mmol) in acetonitrile (5 mL) was added 2,2-difluoro-2-fluorosulfonyl-acetic acid (670 mg, 3.77 mmol). After stirring at 25° C. for 2 h, the reaction was quenched by addition of saturated aqueous sodium bicarbonate (10 mL) and extracted with ethyl acetate (10 mL×3). The combined organic layers were washed with brine (20 mL), dried over anhydrous sodium sulfate, filtered and concentrated under reduced pressure. The residue was purified by column chromatography to afford the title compound. LCMS R_{T}=0.857 min, m/z=311.0 [M+H]⁺.

### Step 3: 1-(2-(difluoromethoxy)ethyl)pyrimidine-2,4(1H,3H)-dione

A mixture of 3-benzoyl-1-(2-(difluoromethoxy)ethyl)pyrimidine-2,4(1H,3H)-dione (230 mg, 0.74 mmol) and sodium methylate (80 mg, 1.48 mmol) in methanol (5 mL) was stirred at 25° C. for 3 h. Then the reaction was diluted with water (5 mL) and extracted with ethyl acetate (5 mL×2). The combined organic layers were washed with brine (5 mL), dried over anhydrous sodium sulfate, filtered and concentrated under reduced pressure. The residue was purified by RP-HPLC to afford the title compound. LCMS R_{T}=0.319 min, m/z=207.0 [M+H]⁺.

### Step 4

Example 61 was prepared from 1-(2-(difluoromethoxy)ethyl)pyrimidine-2,4(1H,3H)-dione and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC. ¹H NMR (400 MHz, CD₃OD) δ=8.71 (d, J=4.8 Hz, 1H), 8.52 (s, 1H), 7.69-7.36 (m, 3H), 7.27-7.05 (m, 2H), 6.67-6.08 (m, 1H), 5.77 (d, J=7.6 Hz, 1H), 5.39 (s, 2H), 4.09 (dd, J=14.8, 4.4 Hz, 4H), 3.79-3.58 (m, 1H), 3.31-2.97 (m, 4H), 2.96-2.22 (m, 4H), 2.15-1.32 (m, 4H). LCMS R_{T}=0.787 min, m/z=588.1 [M+H]⁺.

### Example 62: (S)-3-((7-(6-chloro-1-(pyrrolidin-3-yl)-1,2,3,4-tetrahydroquinolin-8-yl)thieno[3,2-b]pyridin-2-yl)methyl)-1-(cyclopropylmethyl)pyrimidine-2,4(1H,3H)-dione, formic acid salt

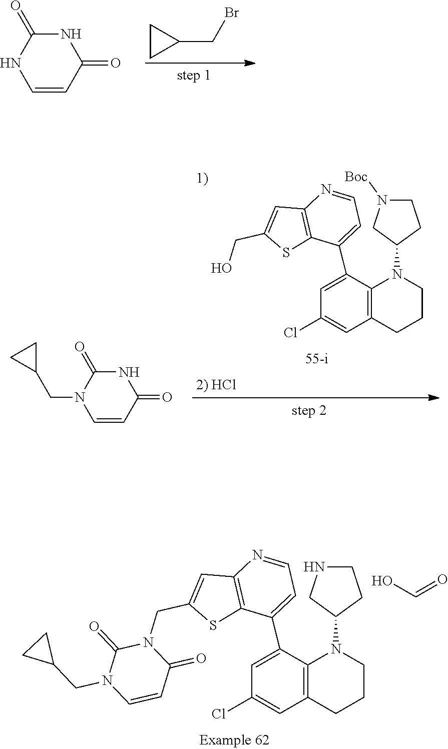

### Step 1: 1-(cyclopropylmethyl)pyrimidine-2,4(1H,3H)-dione

To a solution of pyrimidine-2,4(1H,3H)-dione (4.0 g, 35.69 mmol), (bromomethyl)cyclopropane (4.8 g, 35.69 mmol) and potassium carbonate (9.9 g, 71.37 mmol) in N,N-dimethylformamide (150 mL) was added 1,4,7,10,13,16-hexaoxacyclooctadecane (943 mg, 3.57 mmol). After stirring at 100° C. for 4 h under nitrogen atmosphere, the reaction was filtered and concentrated under reduced pressure. The residue was purified by RP-HPLC to afford the title compound. ¹H NMR (400 MHz, CD₃OD) δ=7.65 (d, J=8.0 Hz, 1H), 5.65 (d, J=8.0 Hz, 1H), 3.61 (d, J=7.2 Hz, 2H), 1.25-1.13 (m, 1H), 0.62-0.54 (m, 2H), 0.41-0.35 (m, 2H).

### Step 2: (S)-3-((7-(6-chloro-1-(pyrrolidin-3-yl)-1,2,3,4-tetrahydroquinolin-8-yl)thieno[3,2-b]pyridin-2-yl)methyl)-1-(cyclopropylmethyl)pyrimidine-2,4(1H,3H)-dione, formic acid salt

Example 62 was prepared from 1-(cyclopropylmethyl)pyrimidine-2,4(1H,3H)-dione and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC. ¹H NMR (400 MHz, CD₃OD) δ=8.70 (d, J=4.8 Hz, 1H), 8.51 (s, 1H), 7.69 (d, J=8.0 Hz, 1H), 7.55-7.42 (m, 2H), 7.22-7.11 (m, 2H), 5.77 (d, J=8.0 Hz, 1H), 5.39 (s, 2H), 3.76-3.62 (m, 3H), 3.29-3.08 (m, 3H), 3.02-2.17 (m, 5H), 2.00-1.47 (m, 4H), 1.26-1.13 (m, 1H), 0.60-0.51 (m, 2H), 0.42-0.34 (m, 2H). LCMS R_{T}=1.660 min, m/z=548.3 [M+H]⁺.

### Example 63: (S)-3-((7-(6-chloro-1-(pyrrolidin-3-yl)-1,2,3,4-tetrahydroquinolin-8-yl)thieno[3,2-b]pyridin-2-yl)methyl)-1-methylpyrimidine-2,4(1H,3H)-dione, formic acid salt

Example 63 was prepared from 1-methylpyrimidine-2,4(1H,3H)-dione and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC. ¹H NMR (400 MHz, CD₃OD) δ=8.70 (d, J=4.8 Hz, 1H), 8.53 (s, 1H), 7.66-7.40 (m, 3H), 7.24-7.08 (m, 2H), 5.76 (d, J=7.6 Hz, 1H), 5.38 (s, 2H), 3.79-3.56 (m, 1H), 3.38 (s, 3H), 3.30-3.03 (m, 4H), 3.01-2.56 (m, 4H), 2.05-1.45 (m, 4H). LCMS R_{T}=1.175 min, m/z=508.3 [M+H]⁺.

### Example 64: 3-((7-(6-chloro-1-((S)-pyrrolidin-3-yl)-1,2,3,4-tetrahydroquinolin-8-yl)thieno[3,2-b]pyridin-2-yl)methyl)-1-((S)-2-hydroxy-3,3- dimethylbutyl)pyrimidine-2,4(1H,3H)-dione, formic acid salt

Example 64 was prepared from (S)-1-(3,3,3-trifluoro-2-hydroxypropyl)pyrimidine-2,4(1H,3H)-dione and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC. ¹H NMR (400 MHz, CD₃OD) δ=8.70 (d, J=4.8 Hz, 1H), 8.50 (s, 1H), 7.66-7.43 (m, 3H), 7.16 (d, J=8.4 Hz, 2H), 5.78 (d, J=8.0 Hz, 1H), 5.38 (s, 2H), 4.40-4.18 (m, 2H), 3.80-3.60 (m, 2H), 3.31-3.00 (m, 5H), 2.96-2.60 (m, 4H), 2.04-1.50 (m, 4H). LCMS R_{T}=0.803 min, m/z=605.9 [M+H]⁺.

### Example 65: 3-((7-(6-chloro-1-((S)-pyrrolidin-3-yl)-1,2,3,4-tetrahydroquinolin-8-yl)thieno[3,2-b]pyridin-2-yl)methyl)-1-((R)-3,3,3-trifluoro-2-hydroxypropyl)pyrimidine-2,4(1H,3H)-dione, formic acid salt

Example 65 was prepared from (R)-1-(3,3,3-trifluoro-2-hydroxypropyl)pyrimidine-2,4(1H,3H)-dione and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC to afford the title compound. ¹H NMR (400 MHz, CD₃OD) δ=8.70 (d, J=4.4 Hz, 1H), 8.51 (s, 1H), 7.69-7.37 (m, 3H), 7.23-7.05 (m, 2H), 5.78 (d, J=7.6 Hz, 1H), 5.38 (s, 2H), 4.28 (d, J=11.2 Hz, 2H), 3.89-3.59 (m, 2H), 3.30-2.99 (m, 4H), 2.93-2.24 (m, 4H), 2.10-1.57 (m, 4H). LCMS R_{T}=0.800 min, m/z=606.0 [M+H]⁺.

### Example 66: (S)-2-((7-(6-chloro-1-(pyrrolidin-3-yl)-1,2,3,4-tetrahydroquinolin-8-yl)thieno[3,2-b]pyridin-2-yl)methyl)-5-(2-methoxyethyl)pyridazin-3(2H)-one, formic acid salt

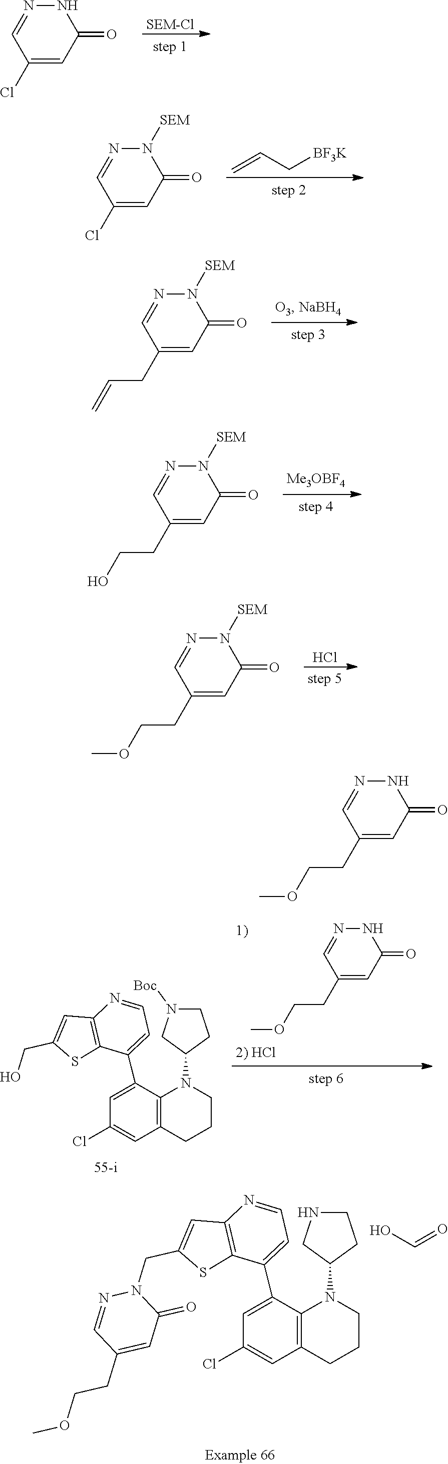

### Step 1: 5-chloro-2-((2-(trimethylsilyl)ethoxy)methyl)pyridazin-3(2H)-one

To a solution of 5-chloropyridazin-3(2H)-one (1.5 g, 11.49 mmol) in N,N-dimethylformamide (100 mL) was added sodium hydride (551 mg, 13.79 mmol, 60% in mineral oil) at 25° C. After stirring at 25° C. for 1 h under nitrogen atmosphere, (2-(chloromethoxy)ethyl)trimethylsilane (2.9 g, 17.24 mmol) was added to the reaction mixture. After stirring at 25° C. for 4 h under nitrogen atmosphere, the reaction was quenched by the addition of water (10 mL). The mixture was diluted with water (20 mL) and extracted with ethyl acetate (20 mL×2). The combined organic layers were washed with brine (20 mL), dried over anhydrous sodium sulfate, filtered and concentrated under reduced pressure. The residue was purified by column chromatography to afford the title compound. ¹H NMR (400 MHz, CD₃OD) δ=7.98 (d, J=2.4 Hz, 1H), 7.14 (d, J=2.4 Hz, 1H), 5.43 (s, 2H), 3.77-3.67 (m, 2H), 0.98-0.89 (m, 2H), 0.07-0.01 (m, 9H).

### Step 2: 5-allyl-2-((2-(trimethylsilyl)ethoxy)methyl)pyridazin-3(2H)-one

To a solution of 5-chloro-2-((2-(trimethylsilyl)ethoxy)methyl)pyridazin-3(2H)-one (1.5 g, 5.75 mmol), potassium allyltrifluoroborate (2.6 g, 17.26 mmol) and cesium carbonate (5.6 g, 17.26 mmol) in dioxane (20 mL) and water (5 mL) was added [2-(2-aminophenyl)phenyl]palladium(II); dicyclohexyl-[2-(2,4,6-triisopropylphenyl)phenyl]phosphane; methanesulfonate (243 mg, 0.29 mmol). After stirring at 100° C. for 3 h under nitrogen atmosphere, the reaction was filtered and the filtrate was concentrated under reduced pressure. The residue was diluted with water (30 mL) and extracted with ethyl acetate (50 mL×2). The combined organic layers were washed with brine (30 mL), dried over anhydrous sodium sulfate, filtered and concentrated under reduced pressure. The residue was purified by column chromatography to afford the title compound. ¹H NMR (400 MHz, DMSO-d₆) δ=7.82 (d, J=2.0 Hz, 1H), 6.74-6.70 (m, 1H), 5.99-5.86 (m, 1H), 5.30 (s, 2H), 5.20-5.13 (m, 2H), 3.64-3.58 (m, 2H), 3.28 (m, 2H), 0.87-0.81 (m, 2H), −0.05 (s, 9H).

### Step 3: 5-(2-hydroxyethyl)-2-((2-(trimethylsilyl)ethoxy)methyl)pyridazin-3(2H)-one

A solution of 5-allyl-2-((2-(trimethylsilyl)ethoxy)methyl)pyridazin-3(2H)-one (450 mg, 1.69 mmol) in dichloromethane (3 mL) and ethanol (1 mL) was stirred at −78° C. under ozone atmosphere for 10 min. The reaction mixture was degassed and purged with nitrogen. Sodium borohydride (255 mg, 6.76 mmol) and ethanol (1 mL) were added to the reaction mixture at −78° C. After stirring at 25° C. for 4 h under nitrogen atmosphere, the reaction mixture was quenched by the addition of methanol (10 mL) and concentrated under reduced pressure. The residue was purified by prep-TLC to afford the title compound. ¹H NMR (400 MHz, CDCl₃) δ=7.83-7.67 (m, 1H), 6.99-6.72 (m, 1H), 5.56-5.36 (m, 2H), 4.05-3.66 (m, 4H), 3.02-2.30 (m, 2H), 2.28-1.29 (m, 2H), 1.04-0.91 (m, 2H), 0.00 (s, 9H).

### Step 4: 5-(2-methoxyethyl)-2-((2-(trimethylsilyl)ethoxy)methyl)pyridazin-3(2H)-one

To a solution of 5-(2-hydroxyethyl)-2-((2-(trimethylsilyl)ethoxy)methyl)pyridazin-3(2H)-one (210 mg, 0.78 mmol), molecular sieves (800 mg) and N1,N1,N8,N8-tetramethylnaphthalene-1,8-diamine (832 mg, 3.88 mmol) in dichloromethane (10 mL) was added trimethyloxonium tetrafluoroborate (574 mg, 3.88 mmol). After stirring at 25° C. for 4 h under nitrogen atmosphere, the reaction was filtered and concentrated under reduced pressure. The residue was diluted with water (30 mL) and extracted with ethyl acetate (50 mL×2). The combined organic layers were washed with brine (30 mL), dried over anhydrous sodium sulfate, filtered and concentrated under reduced pressure. The residue was purified by prep-TLC to afford the title compound. LCMS R_{T}=0.989 min, m/z=284.9 [M+H]⁺.

### Step 5: 5-(2-methoxyethyl)pyridazin-3(2H)-one

To a solution of 5-(2-methoxyethyl)-2-((2-(trimethylsilyl)ethoxy)methyl)pyridazin-3(2H)-one (40 mg, 0.14 mmol) in methanol (0.75 mL) was added concentrated hydrochloric acid (12 M, 0.085 mL). After stirring at 90° C. for 4 h under nitrogen atmosphere, the reaction was filtered and concentrated under reduced pressure. The residue was purified by RP-HPLC to afford the title compound. ¹H NMR (400 MHz, CDCl₃) δ=11.85 (s, 1H), 7.75 (d, J=1.6 Hz, 1H), 6.81 (s, 1H), 3.62 (t, J=6.0 Hz, 2H), 3.35 (s, 3H), 2.75 (t, J=6.0 Hz, 2H).

### Step 6

Example 66 was prepared from 5-(2-methoxyethyl)pyridazin-3(2H)-one and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC. ¹H NMR (400 MHz, CD30D) 6=8.71 (d, J=4.8 Hz, 1H), 8.59-8.43 (m, 1H), 7.93 (d, J=2.0 Hz, 1H), 7.53 (s, 1H), 7.48 (d, J=5.2 Hz, 1H), 7.16 (d, J=7.6 Hz, 2H), 6.85 (s, 1H), 5.64-5.55 (m, 2H), 3.69-3.58 (m, 3H), 3.32 (s, 3H), 3.26-2.42 (m, 10H), 1.92 (q, J=6.0 Hz, 4H). LCMS R_{T}=1.577 min, m/z=536.3 [M+H]⁺.

### Example 67: (S)-3-((7-(6-chloro-1-(pyrrolidin-3-yl)-1,2,3,4-tetrahydroquinolin-8-yl)thieno[3,2-b]pyridin-2-yl)methyl)-1-(3,3-difluoropropyl)pyrimidine-2,4(1H,3H)-dione, formic acid salt

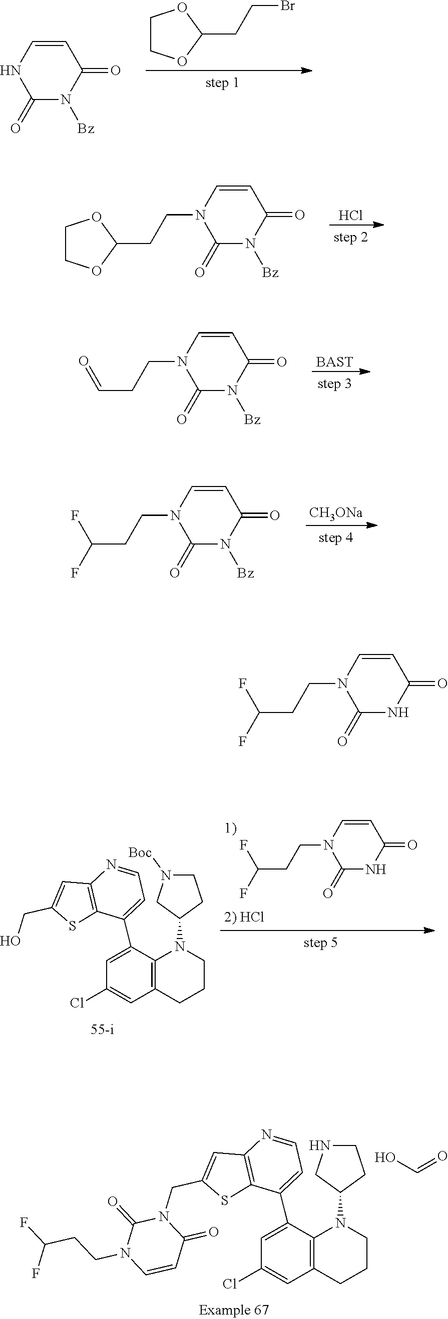

### Step 1: 1-(2-(1,3-dioxolan-2-yl)ethyl)-3-benzoylpyrimidine-2,4(1H,3H)-dione

A mixture of 3-benzoylpyrimidine-2,4(1H,3H)-dione (2.0 g, 9.25 mmol), 2-(2-bromoethyl)-1,3-dioxolane (2.0 g, 11.1 mmol), potassium carbonate (1.5 g, 11.1 mmol) and tetrabutylammonium iodide (171 mg, 0.46 mmol) in N,N-dimethylformamide (50 mL) was stirred at 25° C. for 12 h. The reaction mixture was filtered and concentrated under reduced pressure. The residue was purified by column chromatography to afford the title compound.

### Step 2: 3-(3-benzoyl-2,4-dioxo-3,4-dihydropyrimidin-1(2H)-yl)propanal

A solution of 1-(2-(1,3-dioxolan-2-yl)ethyl)-3-benzoylpyrimidine-2,4(1H,3H)-dione (1.8 g, 5.69 mmol) in concentrated hydrochloric acid (11.86 mL), water (15 mL) and tetrahydrofuran (10 mL) was stirred at 20° C. for 18 h. The reaction mixture was diluted with water (40 mL) and extracted with dichloromethane (20 mL×2). The combined organic layers were washed with brine (40 mL), dried over anhydrous sodium sulfate, filtered and concentrated under reduced pressure to afford the title compound. It was used in the next step without further purification.

### Step 3: 3-benzoyl-1-(3,3-difluoropropyl)pyrimidine-2,4(1H,3H)-dione

To a solution of 3-(3-benzoyl-2,4-dioxo-3,4-dihydropyrimidin-1(2H)-yl)propanal (0.6 g, 2.2 mmol) in dichloromethane (30 mL) was added bis(2-methoxyethyl)amino sulfur trifluoride (4.9 g, 22.04 mmol) at −78° C. After stirring at −78° C. for 1 h, the reaction was quenched by addition of saturated aqueous sodium bicarbonate (30 mL) at 0° C. The mixture was diluted with water (15 mL) and extracted with dichloromethane (25 mL×2). The combined organic layers were dried over anhydrous sodium sulfate, filtered and concentrated under reduced pressure. The residue was purified by prep-TLC to afford the title compound.

### Step 4: 1-(3,3-difluoropropyl)pyrimidine-2,4(1H,3H)-dione

To a solution of 3-benzoyl-1-(3,3-difluoropropyl)pyrimidine-2,4(1H,3H)-dione (130 mg, 0.44 mmol) in methanol (1.5 mL) was added sodium methylate (71 mg, 1.33 mmol). After stirring at 20° C. for 3 h, the reaction was diluted with a solution of HCl in methanol (4 M, 0.5 mL). The mixture was purified by RP-HPLC to afford the title compound.

Step 5: Example 67 was prepared from 1-(3,3-difluoropropyl)pyrimidine-2,4(1H,3H)-dione and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC to give (S)-3-((7-(6-chloro-1-(pyrrolidin-3-yl)-1,2,3,4-tetrahydroquinolin-8-yl)thieno[3,2-b]pyridin-2-yl)methyl)-1-(3,3-difluoropropyl)pyrimidine-2,4(1H,3H)-dione (39.73 mg, 39%). ¹H NMR (400 MHz, CD₃OD) δ=8.70 (d, J=2.8 Hz, 1H), 8.52 (s, 1H), 7.62 (d, J=7.6 Hz, 1H), 7.55-7.39 (m, 2H), 7.16 (d, J=6.4 Hz, 2H), 6.23-5.85 (m, 1H), 5.78 (d, J=8.0 Hz, 1H), 5.37 (s, 2H), 3.99 (t, J=6.8 Hz, 2H), 3.67 (t, J=8.4 Hz, 1H), 3.30-3.01 (m, 4H), 2.94-2.44 (m, 4H), 2.35-2.19 (m, 2H), 2.03-1.44 (m, 4H). LCMS R_{T}=1.570 min, m/z=572.3 [M+H]⁺.

### Example 68: (S)-2-((7-(6-chloro-1-(pyrrolidin-3-yl)-1,2,3,4-tetrahydroquinolin-8-yl)thieno[3,2-b]pyridin-2-yl)methyl)-6,7-dihydro-2H-pyrrolo[2,1-c][1,2,4]triazol-3(5H)-one, formic acid salt

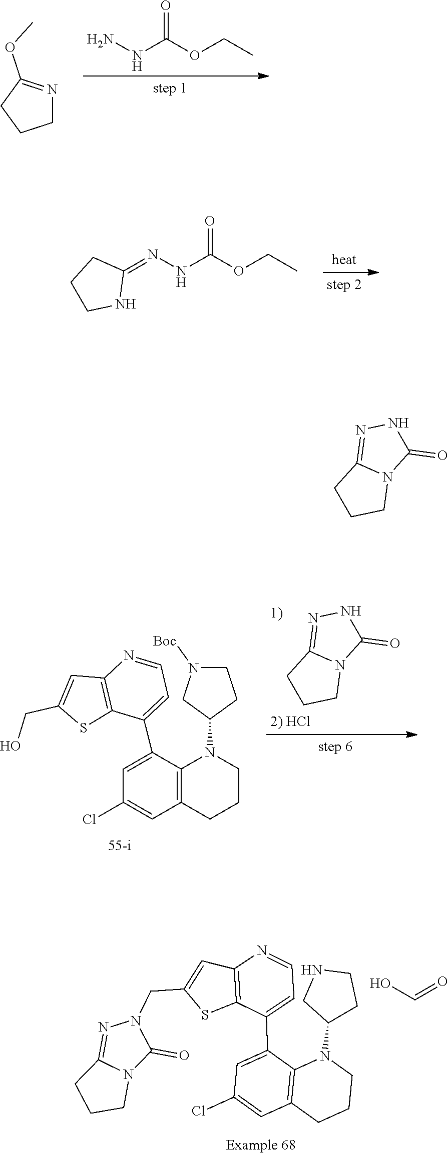

### Step 1: (E)-ethyl 2-(pyrrolidin-2-ylidene)hydrazinecarboxylate

A mixture of 5-methoxy-3,4-dihydro-2H-pyrrole (2.0 g, 20.18 mmol) and ethyl hydrazinecarboxylate (2.1 g, 20.18 mmol) in ethanol (30 mL) and concentrated hydrochloric acid (0.08 mL) was stirred at 80° C. for 12 h. The reaction was concentrated under reduced pressure and the residue purified by column chromatography to afford the title compound.

### Step 2: 6,7-dihydro-2H-pyrrolo[2,1-c][1,2,4]triazol-3(5H)-one

A mixture of (E)-ethyl 2-(pyrrolidin-2-ylidene)hydrazinecarboxylate (2.3 g, 13.43 mmol) in 1,2-dichlorobenzene (35 mL) was stirred at 120° C. for 5 h. The mixture was diluted with water (50 mL) and extracted with dichloromethane (20 mL×2). The aqueous phase was concentrated under reduced pressure. The residue was purified by RP-HPLC to afford the title compound. ¹H NMR (400 MHz, CD₃OD) δ=2.65-2.50 (m, 2H) 2.87-2.73 (m, 2H) 3.81-3.67 (m, 2H). LCMS R_{T}=0.25 min, m/z=126.1 [M+H]⁺.

### Step 3

Example 68 was prepared from 6,7-dihydro-2H-pyrrolo[2,1-c][1,2,4]triazol-3(5H)-one and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC. ¹H NMR (400 MHz, CD₃OD) δ=8.71 (d, J=4.8 Hz, 1H), 8.49 (s, 1H), 7.52-7.41 (m, 2H), 7.15 (d, J=10.4 Hz, 2H), 5.21 (s, 2H), 3.75 (t, J=7.2 Hz, 2H), 3.72-3.60 (m, 1H), 3.31-3.04 (m, 4H), 2.98-2.66 (m, 6H), 2.55 (m, J=7.2 Hz, 2H), 2.04-1.53 (m, 4H). LCMS R_{T}=1.640 min, m/z=507.1 [M+H]⁺.

### Example 69: (S)-3-((7-(6-chloro-1-(pyrrolidin-3-yl)-1,2,3,4-tetrahydroquinolin-8-yl)thieno[3,2-b]pyridin-2-yl)methyl)-1-((1-(difluoromethyl)cyclopropyl)methyl)pyrimidine-2,4(1H,3H)-dione, formic acid salt

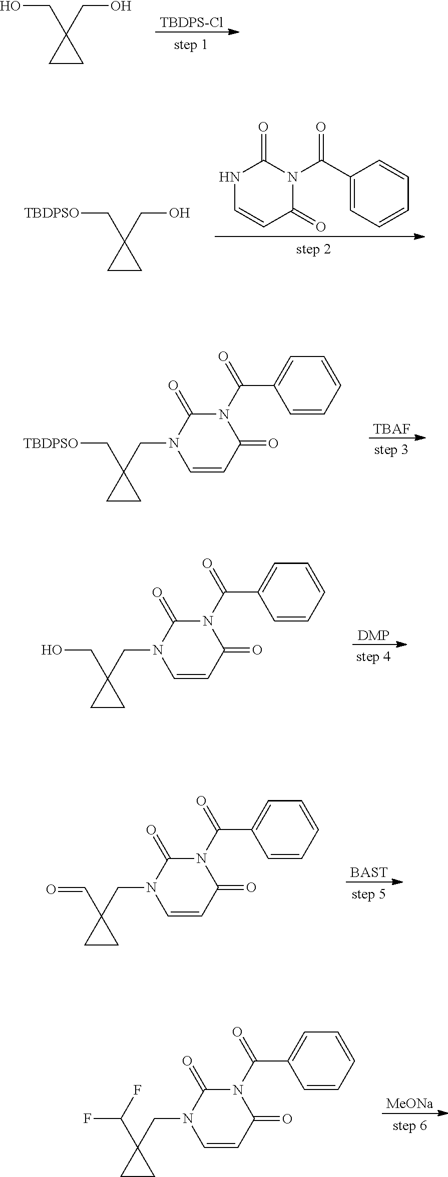

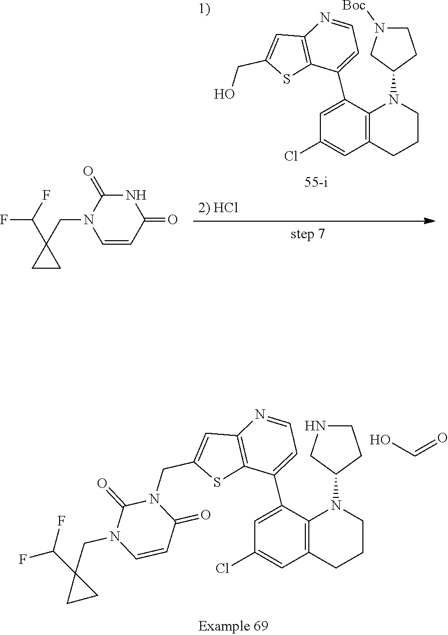

### Step 1: (1-(((tert-butyldiphenylsilyl)oxy)methyl)cyclopropyl)methanol

To a solution of cyclopropane-1,1-diyldimethanol (3.0 g, 29.37 mmol) and imidazole (4.0 g, 58.75 mmol) in N,N-dimethylformamide (50 mL) was added tert-butylchlorodiphenylsilane (8.1 g, 29.37 mmol) at 0° C. After stirring at 20° C. for 12 h, the reaction was quenched by addition of water (300 mL) and extracted with ethyl acetate (100 mL×2). The combined organic layers were washed with brine (10 mL), dried over anhydrous sodium sulfate, filtered and concentrated under reduced pressure. The residue was purified by column chromatography to afford the title compound. LCMS R_{T}=1.023 min, m/z=340.9 [M+H]⁺.

### Step 2: 3-benzoyl-1-((1-(((tert-butyldiphenylsilyl)oxy)methyl)cyclopropyl)methyl)pyrimidine-2,4(1H,3H)-dione

To a solution of 3-benzoylpyrimidine-2,4(1H,3H)-dione (2.5 g, 11.75 mmol), (1-(((tert-butyldiphenylsilyl)oxy)methyl)cyclopropyl)methanol (2.0 g, 5.87 mmol) and triphenylphosphine (3.1 g, 11.75 mmol) in tetrahydrofuran (10 ml) was added diisopropyl azodicarboxylate (2.4 g, 11.75 mmol). After stirring at 50° C. for 0.5 h under nitrogen atmosphere, the reaction was quenched by addition of water (50 ml) and extracted with ethyl acetate (30 ml×3). The combined organic layers were washed with brine (10 ml), dried over anhydrous sodium sulfate, filtered and concentrated under reduced pressure. The residue was purified by column chromatography to afford the title compound. LCMS R_{T}=1.180 min, m/z=539.3 [M+H]⁺.

### Step 3: 3-benzoyl-1-((1-(hydroxymethyl)cyclopropyl)methyl)pyrimidine-2,4(1H,3H)-dione

To a solution of 3-benzoyl-1-((1-(((tert-butyldiphenylsilyl)oxy)methyl)cyclopropyl)methyl)pyrimidine-2,4(1H,3H)-dione (3.0 g, 5.57 mmol) in tetrahydrofuran (20 mL) was added tetrabutylammonium fluoride (1 M in tetrahydrofuran, 5.57 mL). After stirring at 20° C. for 0.5 h, the reaction was concentrated under reduced pressure. The residue was purified by column chromatography to afford the title compound. LCMS R_{T}=0.704 min, m/z=301.1 [M+H]⁺.

### Step 4: 1-((3-benzoyl-2,4-dioxo-3,4-dihydropyrimidin-1(2H)-yl)methyl)cyclopropanecarbaldehyde

To a solution of 3-benzoyl-1-((1-(hydroxymethyl)cyclopropyl)methyl)pyrimidine-2,4(1H,3H)-dione (400 mg, 1.33 mmol) in dichloromethane (10 mL) was added 1,1,1-triacetoxy-1,1-dihydro-1,2-benziodoxol-3(1H)-one (1.2 g, 2.66 mmol) at 0° C. After stirring at 20° C. for 1 h, the reaction was quenched by addition of water (10 mL) and extracted with ethyl acetate (10 mL×3). The combined organic layers were washed with brine (10 mL), dried over anhydrous sodium sulfate, filtered and concentrated under reduced pressure. The residue was purified by column chromatography to afford the title compound.

### Step 5: 3-benzoyl-1-((1-(difluoromethyl)cyclopropyl)methyl)pyrimidine-2,4(1H,3H)-dione

To a solution of 1-((3-benzoyl-2,4-dioxo-3,4-dihydropyrimidin-1(2H)-yl)methyl)cyclopropanecarbaldehyde (400 mg, 1.34 mmol) in dichloromethane (3 mL) was added bis(2-methoxyethyl)amino sulfurtrifluoride (3.0 g, 13.41 mmol). After stirring at 20° C. for 1 h, the reaction was quenched by addition of water (10 mL) and extracted with ethyl acetate (10 mL×3). The combined organic layers were washed with brine (10 mL), dried over anhydrous sodium sulfate, filtered and concentrated under reduced pressure. The residue was purified by column chromatography to afford the title compound. LCMS R_{T}=1.033 min, m/z=320.8 [M+H]⁺.

### Step 6: 1-((1-(difluoromethyl)cyclopropyl)methyl)pyrimidine-2,4(1H,3H)-dione

To a solution of 3-benzoyl-1-((1-(difluoromethyl)cyclopropyl)methyl)pyrimidine-2,4(1H,3H)-dione (300 mg, 0.94 mmol) in methanol (3 mL) was added sodium methylate (151 mg, 2.81 mmol). After stirring at 20° C. for 3 h, the reaction mixture was purified by RP-HPLC to afford the title compound. LCMS R_{T}=1.053 min, m/z=216.9 [M+H]⁺.

### Step 7

Example 69 was prepared from 1-((1-(difluoromethyl)cyclopropyl)methyl)pyrimidine-2,4(1H,3H)-dione and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC. ¹H NMR (400 MHz, CD₃OD) δ=8.70 (d, J=4.8 Hz, 1H), 8.51 (s, 1H), 7.63 (d, J=8.0 Hz, 1H), 7.51 (s, 1H), 7.47 (d, J=4.8 Hz, 1H), 7.19-7.14 (m, 2H), 5.82-5.49 (m, 2H), 5.42-5.33 (m, 2H), 4.00 (s, 2H), 3.67 (q, J=8.4 Hz, 1H), 3.29-3.02 (m, 4H), 2.88 (t, J=6.1 Hz, 2H), 2.80-2.39 (m, 2H), 1.93 (q, J=6.2 Hz, 2H), 1.86-1.18 (m, 2H), 0.97-0.89 (m, 2H), 0.88-0.82 (m, 2H). LCMS R_{T}=1.032 min, m/z=598.4 [M+H]⁺.

### Example 70: (S)-1-((3-((7-(6-chloro-1-(pyrrolidin-3-yl)-1,2,3,4-tetrahydroquinolin-8-yl)thieno[3,2-b]pyridin-2-yl)methyl)-2,4-dioxo-3,4-dihydropyrimidin-1(2H)-yl)methyl)cyclopropanecarbonitrile, formic acid salt

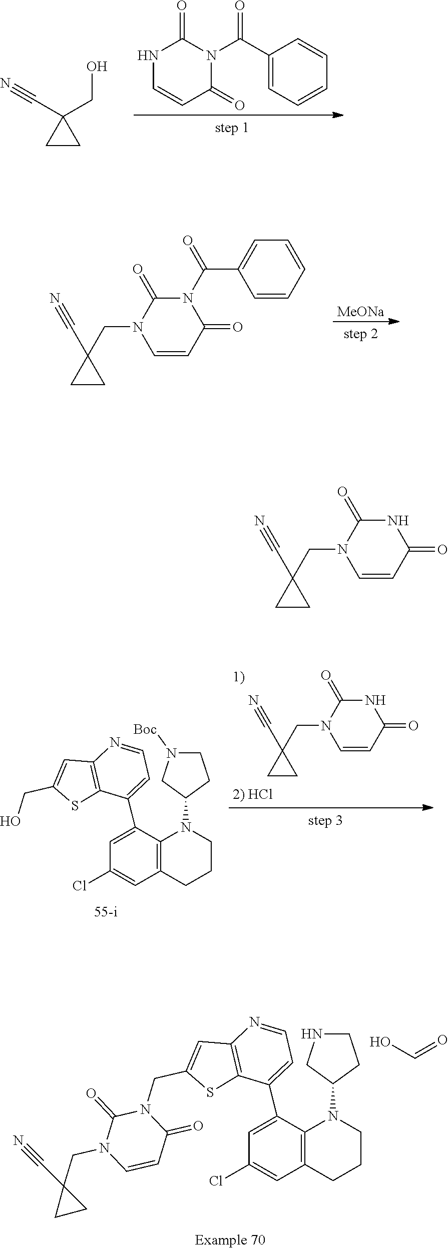

### Step 1: 1-((3-benzoyl-2,4-dioxo-3,4-dihydropyrimidin-1(2H)-yl)methyl)cyclopropanecarbonitrile

To a solution of 1-(hydroxymethyl)cyclopropanecarbonitrile (200 mg, 2.06 mmol), 3-benzoylpyrimidine-2,4(1H,3H)-dione (668 mg, 3.09 mmol) and triphenylphosphine (1.4 g, 5.15 mmol) in tetrahydrofuran (20 mL) was added diisopropyl azodicarboxylate (1.0 g, 5.15 mmol) at 0° C. After stirring at 0° C. for 1.5 h, the reaction was concentrated under reduced pressure. The residue was purified by column chromatography to afforded the title compound.

### Step 2: 1-((2,4-dioxo-3,4-dihydropyrimidin-1(2H)-yl)methyl)cyclopropanecarbonitrile

To a solution of 1-((3-benzoyl-2,4-dioxo-3,4-dihydropyrimidin-1(2H)-yl)methyl)cyclopropanecarbonitrile (600 mg, 2.03 mmol) in methanol (8 mL) was added sodium methylate (329 mg, 6.1 mmol). After stirring at 20° C. for 12 h, the reaction was concentrated under reduced pressure. The residue was purified by RP-HPLC to give 1-((2,4-dioxo-3,4-dihydropyrimidin-1(2H)-yl)methyl)cyclopropanecarbonitrile.

### Step 3

Example 70 was prepared from 1-((2,4-dioxo-3,4-dihydropyrimidin-1(2H)-yl)methyl)cyclopropanecarbonitrile and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC to afford the title compound. ¹H NMR (400 MHz, CD₃OD) δ=8.70 (d, J=4.8 Hz, 1H), 7.74 (d, J=8.0 Hz, 1H), 7.56-7.40 (m, 2H), 7.17 (s, 2H), 5.85 (d, J=8.0 Hz, 1H), 5.44-5.33 (m, 2H), 3.97 (s, 2H), 3.66 (q, J=8.4 Hz, 1H), 3.31-3.02 (m, 4H), 2.92-2.40 (m, 4H), 1.98-1.45 (m, 4H), 1.30 (s, 4H). LCMS R_{T}=1.477 min, m/z=573.4 [M+H]⁺.

### Example 71: (S)-2-((7-(6-chloro-1-(pyrrolidin-3-yl)-1,2,3,4-tetrahydroquinolin-8-yl)thieno[3,2-b]pyridin-2-yl)methyl)isoindolin-1-one, formic acid salt

Example 71 was prepared from isoindolin-1-one and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC. ¹H NMR (400 MHz, CD₃OD) δ=8.72 (d, J=4.8 Hz, 1H), 8.52 (s, 1H), 7.82 (d, J=7.6 Hz, 1H), 7.69-7.40 (m, 5H), 7.13 (s, 2H), 5.24-5.01 (m, 2H), 4.66-4.47 (m, 2H), 3.64 (q, J=8.4 Hz, 1H), 3.30-2.92 (m, 4H), 2.88-2.51 (m, 4H), 2.01-1.44 (m, 4H). LCMS R_{T}=0.810 min, m/z=515.2 [M+H]⁺.

### Example 72: (S)-3-((7-(6-chloro-1-(pyrrolidin-3-yl)-1,2,3,4-tetrahydroquinolin-8-yl)thieno[3,2-b]pyridin-2-yl)methyl)-1-cyclopropylpyrimidine-2,4(1H,3H)-dione

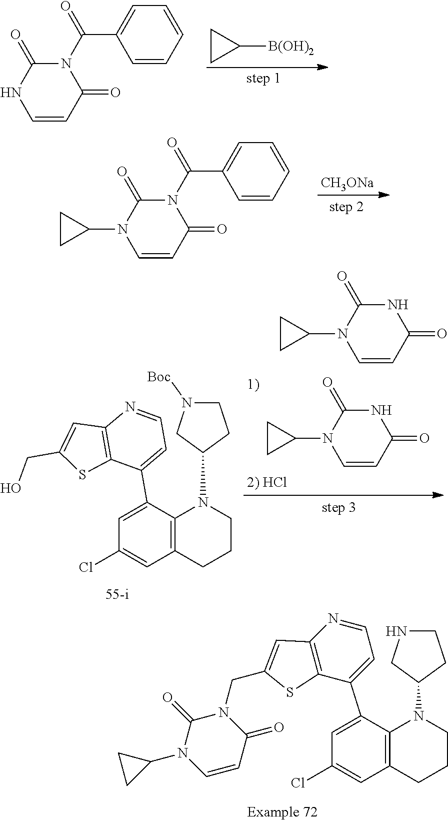

### Step 1: 3-benzoyl-1-cyclopropylpyrimidine-2,4(1H,3H)-dione

To a solution of 3-benzoyl-1H-pyrimidine-2,4-dione (1.2 g, 5.55 mmol) and cyclopropylboronic acid (953 mg, 11.1 mmol) in 1,2-dichloroethane (5 mL) were added copper acetate (1.0 g, 5.55 mmol), sodium carbonate (1.2 g, 11.1 mmol) and 2,2′-bipyridine (693 mg, 4.44 mmol). After stirring at 70° C. for 12 h, the reaction was filtered. The filtrate was diluted with water (15 mL) and extracted with ethyl acetate (15 mL×3). The combined organic layers were washed with brine (15 mL), dried over anhydrous sodium sulfate, filtered and concentrated under reduced pressure. The residue was purified by column chromatography to afford the title compound. LCMS R_{T}=0.820 min, m/z=279.1 [M+Na]⁺.

### Step 2: 1-cyclopropylpyrimidine-2,4(1H,3H)-dione

To a solution of 3-benzoyl-1-cyclopropylpyrimidine-2,4(1H,3H)-dione (160 mg, 0.62 mmol) in methanol (8 mL) was added sodium methylate (101 mg, 1.87 mmol). After stirring at 25° C. for 2 h, the reaction was concentrated under reduced pressure. The residue was purified by RP-HPLC to afford the title compound. LCMS R_{T}=0.695 min, m/z=153.3 [M+H]⁺.

### Step 3

Example 72 was prepared from 1-cyclopropylpyrimidine-2,4(1H,3H)-dione and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC. ¹H NMR (400 MHz, CD₃OD) δ=8.75 (s, 1H), 7.60 (d, J=7.2 Hz, 1H), 7.50 (d, J=16.4 Hz, 2H), 7.16 (d, J=8.4 Hz, 2H), 5.73 (d, J=7.2 Hz, 1H), 5.36 (s, 2H), 3.68 (s, 1H), 3.29-3.08 (m, 5H), 2.88 (s, 2H), 1.93 (s, 3H), 1.50-1.20 (m, 2H), 1.03 (d, J=5.6 Hz, 2H), 0.86 (s, 2H), 0.92-0.80 (m, 1H). LCMS R_{T}=0.752 min, m/z=534.3 [M+H]⁺.

### Example 73: (S)-3-((7-(6-chloro-1-(pyrrolidin-3-yl)-1,2,3,4-tetrahydroquinolin-8-yl)thieno[3,2-b]pyridin-2-yl)methyl)oxazolidine-2,4-dione, formic acid salt

Example 73 was prepared from oxazolidine-2,4-dione and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC. ¹H NMR (400 MHz, CD30D) 6=8.74 (d, J=4.8 Hz, 1H), 8.50 (s, 1H), 7.59-7.47 (m, 2H), 7.19 (d, J=3.6 Hz, 2H), 5.01 (s, 2H), 4.89-4.87 (m, 2H), 3.69 (q, J=8.4 Hz, 1H), 3.26-3.10 (m, 3H), 3.03-2.18 (m, 5H), 2.10-1.57 (m, 4H). LCMS R_{T}=1.469 min, m/z=483.0 [M+H]⁺.

### Example 74: (S)-3-((7-(6-chloro-1-(pyrrolidin-3-yl)-1,2,3,4-tetrahydroquinolin-8-yl)thieno[3,2-b]pyridin-2-yl)methyl)-1-methylimidazolidine-2,4-dione, formic acid salt

Example 74 was prepared from 1-methylimidazolidine-2,4-dione and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC. ¹H NMR (400 MHz, CD30D) 6=8.71 (d, J=5.2 Hz, 1H), 8.53 (s, 1H), 7.48-7.47 (m, 2H), 7.17-7.15 (m, 2H), 4.95 (s, 2H), 4.02 (s, 2H), 3.70-3.61 (m, 1H), 3.28-3.02 (m, 4H), 2.98 (s, 3H), 2.88 (t, J=6.4 Hz, 2H), 2.68 (s, 2H), 2.10-1.60 (m, 4H). LCMS R_{T}=1.384 min, m/z=496.1 [M+H]⁺.

### Example 75: (S)-3-((7-(6-chloro-1-(pyrrolidin-3-yl)-1,2,3,4-tetrahydroquinolin-8-yl)thieno[3,2-b]pyridin-2-yl)methyl)-1-(3,3-difluorocyclobutyl)pyrimidine-2,4(1H,3mH-dione

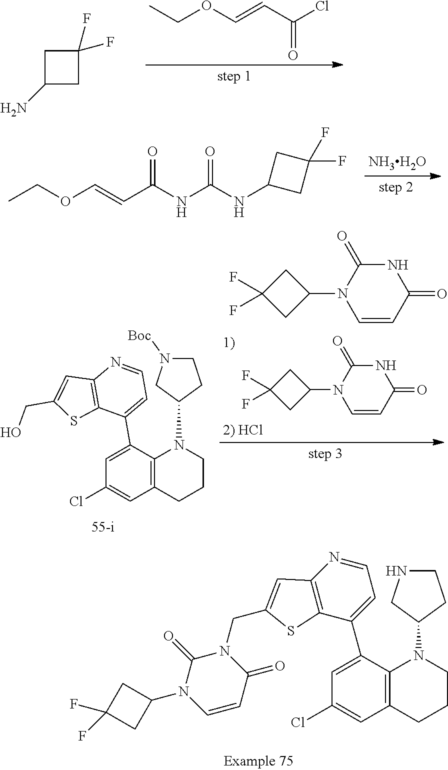

### Step 1: (E)-N-((3,3-difluorocyclobutyl)carbamoyl)-3-ethoxyacrylamide

A solution of 3,3-difluorocyclobutanamine; hydrochloride (300 mg, 2.09 mmol) and cyanatosilver (1.9 g, 12.54 mmol) in toluene (10 mL) was stirred at 95° C. for 0.5 hr. The reaction mixture was cooled to 0° C. and a solution of (E)-3-ethoxyacryloyl chloride (300 mg, 2.09 mmol) in N,N-dimethylformamide (5 mL) was added. After stirring at 20° C. for 0.5 h, the reaction was diluted with saturated aqueous sodium bicarbonate (50 mL) and extracted with ethyl acetate (25 mL×2). The combined organic layers were washed with brine (50 mL), dried over anhydrous sodium sulfate, filtered and concentrated under reduced pressure to give the crude title compound. It was used in the next step without further purification.

### Step 2: 1-(3,3-difluorocyclobutyl)pyrimidine-2,4(1H,3H)-dione

A solution of (E)-N-((3,3-difluorocyclobutyl)carbamoyl)-3-ethoxyacrylamide (1.0 g, 4.03 mmol) in ethanol (10 mL), dioxane (6 mL) and concentrated ammonium hydroxide solution (8 mL) was stirred at 100° C. for 12 h. The reaction was concentrated under reduced pressure. The residue was purified by RP-HPLC to afford the title compound. ¹H NMR (400 MHz, CD₃OD) δ=7.64 (d, J=8.0 Hz, 1H), 5.69 (d, J=8.0 Hz, 1H), 4.62-4.57 (m, 1H), 3.13-2.94 (m, 4H).

### Step 3

Example 75 was prepared from 1-(3,3-difluorocyclobutyl)pyrimidine-2,4(1H,3H)-dione and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 1. The final compound was purified by RP-HPLC. ¹H NMR (400 MHz, CD₃OD) δ=8.66 (d, J=4.8 Hz, 1H), 7.65 (d, J=8.0 Hz, 1H), 7.51 (s, 1H), 7.43 (d, J=4.8 Hz, 1H), 7.11 (s, 2H), 5.81 (d, J=8.0 Hz, 1H), 5.36 (s, 2H), 4.69-4.55 (m, 1H), 3.56-3.43 (m, 1H), 3.26-3.12 (m, 2H), 3.11-2.02 (m, 10H), 2.00-1.86 (m, 2H), 1.76-1.06 (m, 2H). LCMS R_{T}=1.495 min, m/z=584.3 [M+H]⁺.

### Example 76: (S)-3-((7-(6-chloro-1-(pyrrolidin-3-yl)-1,2,3,4-tetrahydroquinolin-8-yl)thieno[3,2-b]pyridin-2-yl)methyl)-1-methyldihydropyrimidine-2,4(1H,3H)-dione, formic acid salt

To a solution of (S)-tert-butyl 3-(6-chloro-8-(2-(hydroxymethyl)thieno[3,2-b]pyridin-7-yl)-3,4-dihydroquinolin-1(2H)-yl)pyrrolidine-1-carboxylate (55-i, 60 mg, 0.12 mmol), 1-methyldihydropyrimidine-2,4(1H,3H)-dione (15 mg, 0.12 mmol) and 1,1′-azobis(N,N-dimethylformamide) (41 mg, 0.24 mmol) in toluene (2 mL) was added tributylphosphane (97 mg, 0.48 mmol,) at 0° C. After stirring at 100° C. for 1 h, the reaction was concentrated under reduced pressure. The residue was dissolved in dioxane (5 ml) and hydrochloric acid (4 M in dioxane, 1.8 mL) was added. After stirring at 25° C. for 0.5 h, the reaction was concentrated under reduced pressure. The residue was purified by RP-HPLC to afford the title compound. ¹H NMR (400 MHz, CD₃OD) δ=8.70 (d, J=4.8 Hz, 1H), 8.47 (s, 1H), 7.49-7.42 (m, 2H), 7.20-7.14 (m, 2H), 5.20 (s, 2H), 3.74-3.63 (m, 1H), 3.44 (t, J=6.8 Hz, 2H), 3.30-3.11 (m, 4H), 3.03 (s, 3H), 2.79 (t, J=6.8 Hz, 4H), 2.89 (t, J=6.4 Hz, 2H), 1.95 (q, J=6.0 Hz, 4H). LCMS R_{T}=1.158 min, m/z=510.0 [M+H]⁺.

### Example 77: (S)-3-((7-(6-chloro-1-(pyrrolidin-3-yl)-1,2,3,4-tetrahydroquinolin-8-yl)thieno[3,2-b]pyridin-2-yl)methyl)-1-(2,2,2-trifluoroethyl)dihydropyrimidine-2,4(1H,3H)-dione, formic acid salt

Example 77 was prepared from 1-(2,2,2-trifluoroethyl)dihydropyrimidine-2,4(1H,3H)-dione and (S)-tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate (55-i) following the procedure described in the synthesis of Example 76. The final compound was purified by RP-HPLC. ¹H NMR (400 MHz, CD₃OD) δ=8.70 (d, J=4.8 Hz, 1H), 8.52 (s, 1H), 7.51-7.41 (m, 2H), 7.17 (d, J=6.0 Hz, 2H), 5.22 (s, 2H), 4.19 (q, J=8.8 Hz, 2H), 3.72-3.53 (m, 3H), 3.27-3.04 (m, 3H), 2.95-2.59 (m, 6H), 2.02-1.51 (m, 4H). LCMS R_{T}=1.020 min, m/z=578.3 [M+H]⁺.

### Example 78: (R)-1-((7-(1-(pyrrolidin-3-yl)-6-(trifluoromethyl)-1,2,3,4-tetrahydroquinolin-8-yl)thieno[3,2-b]pyridin-2-yl)methyl)pyrrolidine-2,5-dione, formic acid salt

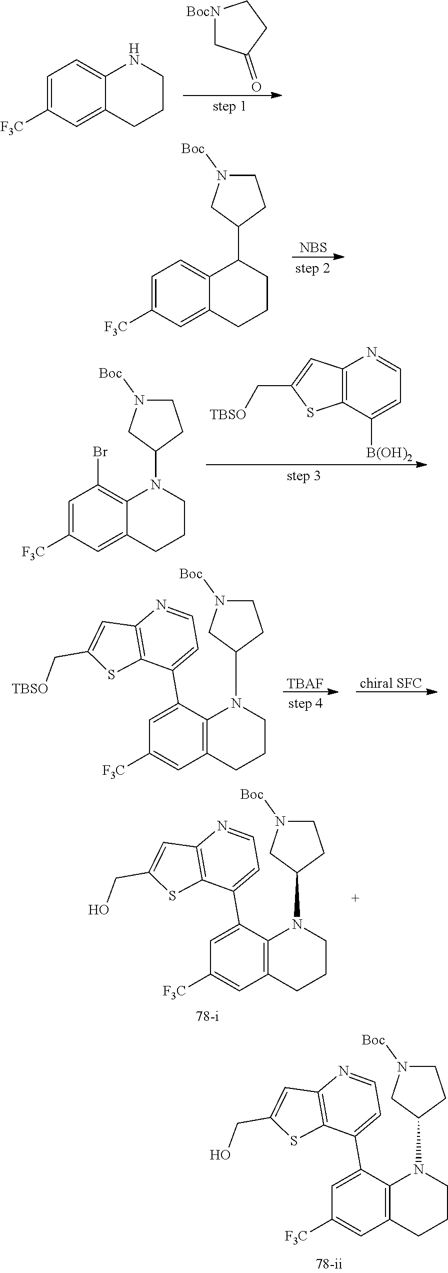

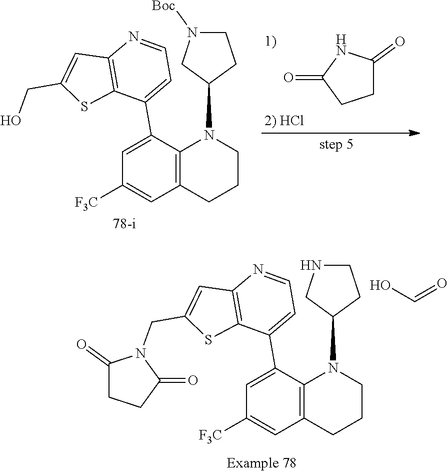

### Step 1: tert-butyl 3-(6-(trifluoromethyl)-3,4-dihydroquinolin-1(2H)-yl)pyrrolidine-1-carboxylate

To a solution of 6-(trifluoromethyl)-1,2,3,4-tetrahydroquinoline (1.7 g, 8.35 mmol) and tert-butyl 3-oxopyrrolidine-1-carboxylate (1.7 g, 9.19 mmol) in acetic acid (40 mL) was added sodium triacetoxyborohydride (5.3 g, 25.05 mmol). After stirring at 20° C. for 12 h, the reaction was diluted with aqueous sodium bicarbonate solution (700 mL) and extracted with ethyl acetate (200 mL×2). The combined organic layers were dried over anhydrous sodium sulfate, filtered and concentrated under reduced pressure. The residue was purified by column chromatography to afford the title compound. LCMS R_{T}=2.680 min, m/z=371.0 [M+H]⁺.

### Step 2: tert-butyl 3-(8-bromo-6-(trifluoromethyl)-3,4-dihydroquinolin-1(2H)-yl)pyrrolidine-1-carboxylate

To a solution of tert-butyl 3-[6-(trifluoromethyl)-3,4-dihydro-2H-quinolin-1-yl]pyrrolidine-1-carboxylate (650 mg, 1.75 mmol) in N,N-dimethylformamide (10 mL) was added N-bromosuccinimide (312 mg, 1.75 mmol). After stirring at 20° C. for 1 h, the reaction was diluted with water (100 mL) and extracted with ethyl acetate (50 mL×2). The combined organic layers were dried over anhydrous sodium sulfate, filtered and concentrated under reduced pressure. The residue was purified by column chromatography to afford the title compound. LCMS R_{T}=2.487 min, m/z=450.9 [M+H]⁺.

### Step 3: tert-butyl 3-(8-(2-(((tert-butyldimethylsilyl)oxy)methyl)thieno[3,2-b]pyridin-7-yl)-6-(trifluoromethyl)-3,4-dihydroquinolin-1(2H)-yl)pyrrolidine-1-carboxylate

To a solution of tert-butyl 3-(8-bromo-6-(trifluoromethyl)-3,4-dihydroquinolin-1(2H)-yl)pyrrolidine-1-carboxylate (520 mg, 1.16 mmol) and (2-(((tert-butyldimethylsilyl)oxy)methyl)thieno[3,2-b]pyridin-7-yl)boronic acid (524 mg, 1.62 mmol) in dioxane (15 mL) and water (1 mL) were added dichloro[1,1′-bis(diphenylphosphino)ferrocene]palladium(II) (169 mg, 0.23 mmol) and potassium carbonate (400 mg, 2.89 mmol). After stirring at 100° C. for 1 h, the reaction was concentrated under reduced pressure. The residue was purified by column chromatography to afford the title compound. LCMS R_{T}=2.957 min, m/z=648.4 [M+H]⁺.

### Step 4: (R)-tert-butyl 3-(8-(2-(hydroxymethyl)thieno[3,2-b]pyridin-7-yl)-6-(trifluoromethyl)-3,4-dihydroquinolin-1(2H)-yl)pyrrolidine-1-carboxylate and (S)-tert-butyl 3-(8-(2-(hydroxymethyl)thieno[3,2-b]pyridin-7-yl)-6-(trifluoromethyl)-3,4-dihydroquinolin-1(2H)-yl)pyrrolidine-1-carboxylate

To a solution of tert-butyl 3-(8-(2-(((tert-butyldimethylsilyl)oxy)methyl)thieno[3,2-b]pyridin-7-yl)-6-(trifluoromethyl)-3,4-dihydroquinolin-1(2H)-yl)pyrrolidine-1-carboxylate (700 mg, 1.08 mmol) in tetrahydrofuran (2 mL) was added tetrabutyl ammonium fluoride (1 M in tetrahydrofuran, 1.62 mL). After stirring at 20° C. for 0.5 h, the reaction was concentrated under reduced pressure. The residue was separated by SFC to give first eluting fraction (78-i, Rt=3.548 min) and second eluting fraction (78-ii, Rt=4.586 min). LCMS R_{T}=2.563 min, m/z=534.1 [M+H]⁺.

### Step 5

To a solution of 78-i (40 mg, 0.07 mmol) and succinimide (22 mg, 0.22 mmol) in tetrahydrofuran (1 mL) were added triphenylphosphine (59 mg, 0.22 mmol) and diisopropylazodicarboxylate (45 mg, 0.22 mmol) at 0° C. After stirring at 20° C. for 1 h, the reaction was concentrated under reduced pressure. The residue was dissolved in dichloromethane (3 mL) and added trifluoroacetic acid (924 mg, 8.1 mmol). After stirring at 20° C. for 1 h, the reaction was concentrated under reduced pressure. The residue was purified by RP-HPLC to afford the title compound (2.95 mg, 4%). ¹H NMR (400 MHz, CD₃OD) δ=8.75 (d, J=4.8 Hz, 1H), 8.52 (s, 1H), 7.53 (d, J=4.4 Hz, 1H), 7.49 (s, 1H), 7.43 (s, 1H), 7.40 (s, 1H), 4.95 (s, 2H), 3.81 (m, J=8.4 Hz, 1H), 3.13 (s, 4H), 3.03-2.28 (m, 8H), 2.08-1.50 (m, 4H). LCMS R_{T}=1.003 min, m/z=515.3 [M+H]⁺. The absolute configuration was tentatively assigned based on the relative potency of the pair enantiomers in biology assays.

### Example 79: (S)-1-((7-(1-(pyrrolidin-3-yl)-6-(trifluoromethyl)-1,2,3,4-tetrahydroquinolin-8-yl)thieno[3,2-b]pyridin-2-yl)methyl)pyrrolidine-2,5-dione, formic acid salt

Example 79 was prepared from 78-ii and succinimide, following the procedure described in the synthesis of Example 78. The final compound was purified by RP-HPLC. ¹H NMR (400 MHz, CD₃OD) 6=8.74 (d, J=4.4 Hz, 1H), 8.52 (s, 1H), 7.53 (d, J=4.0 Hz, 1H), 7.49 (s, 1H), 7.41 (d, J=13.2 Hz, 2H), 4.95 (s, 2H), 3.80 (t, J=8.0 Hz, 1H), 3.31-2.83 (m, 6H), 2.80-2.31 (m, 6H), 2.04-1.30 (m, 4H). LCMS R_{T}=1.005 min, m/z=515.3 [M+H]⁺. The absolute configuration was tentatively assigned based on the relative potency of the pair enantiomers in biology assays.

### Example 80: (R)-1-(2,2-difluoroethyl)-3-((7-(1-(pyrrolidin-3-yl)-6-(trifluoromethyl)-1,2,3,4-tetrahydroquinolin-8-yl)thieno[3,2-b]pyridin-2-yl)methyl)pyrimidine-2,4(1H,3H)-dione

Example 80 was prepared from 78-i and 1-(2,2-difluoroethyl)pyrimidine-2,4(1H,3H)-dione, following the procedure described in the synthesis of Example 78. The final compound was purified by RP-HPLC. ¹H NMR (400 MHz, CD₃OD) δ=8.70 (d, J=4.8 Hz, 1H), 7.64-7.44 (m, 3H), 7.36 (s, 2H), 6.31-5.95 (m, 1H), 5.82 (d, J=8.0 Hz, 1H), 5.38 (s, 2H), 5.43-5.32 (m, 1H), 4.30-4.13 (m, 2H), 3.71-3.55 (m, 1H), 3.30-2.15 (m, 7H), 2.05-1.07 (m, 4H). LCMS R_{T}=1.442 min, m/z=592.3 [M+H]⁺. The absolute configuration was tentatively assigned based on the relative potency of the pair enantiomers in biology assays.

### Example 81: (S)-1-(2,2-difluoroethyl)-3-((7-(1-(pyrrolidin-3-yl)-6-(trifluoromethyl)-1,2,3,4-tetrahydroquinolin-8-yl)thieno[3,2-b]pyridin-2-yl)methyl)pyrimidine-2,4(1H,3H)-dione

Example 81 was prepared from 78-ii and 1-(2,2-difluoroethyl)pyrimidine-2,4(1H,3H)-dione, following the procedure described in the synthesis of Example 78. The final compound was purified by RP-HPLC. ¹H NMR (400 MHz, CD₃OD) δ=8.70 (d, J=4.8 Hz, 1H), 7.65-7.44 (m, 3H), 7.36 (s, 2H), 6.32-5.95 (m, 1H), 5.82 (d, J=8.0 Hz, 1H), 5.38 (s, 2H), 5.43-5.32 (m, 1H), 4.30-4.13 (m, 2H), 3.62 (d, J=7.2 Hz, 1H), 3.29-2.14 (m, 7H), 2.06-1.18 (m, 4H). LCMS R_{T}=1.458 min, m/z=592.3 [M+H]⁺. The absolute configuration was tentatively assigned based on the relative potency of the pair enantiomers in biology assays.

### Example 82: (S)-8-(2-((2,5-dioxopyrrolidin-1-yl)methyl)thieno[3,2-b]pyridin-7-yl)-1-(pyrrolidin-3-yl)-1,2,3,4-tetrahydroquinoline-6-carbonitrile, formic acid salt

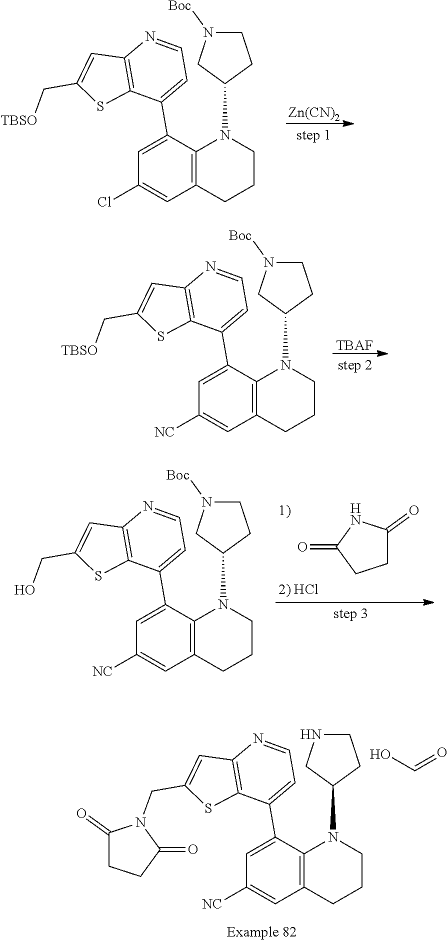

### Step 1: (S)-tert-butyl 3-(8-(2-(((tert-butyldimethylsilyl)oxy)methyl)thieno[3,2-b]pyridin-7-yl)-6-cyano-3,4-dihydroquinolin-1(2H)-yl)pyrrolidine-1-carboxylate

To a solution of (S)-tert-butyl 3-(8-(2-(((tert-butyldimethylsilyl)oxy)methyl)thieno[3,2-b]pyridin-7-yl)-6-chloro-3,4-dihydroquinolin-1(2H)-yl)pyrrolidine-1-carboxylate (500 mg, 0.81 mmol) in N,N-dimethylformamide (8 mL) was added cesium carbonate (795 mg, 2.44 mmol), zinc cyanide (286 mg, 2.44 mmol) and BrettPhos Pd G3 (369 mg, 0.41 mmol). The mixture was stirred at 100° C. for 16 h. The reaction was diluted with water (60 mL) and extracted with ethyl acetate (20 mL×3). The combined organic layers were washed with brine (30 mL), dried over anhydrous sodium sulfate, filtered and concentrated under reduced pressure. The residue was purified by column chromatography to afford the title compound. LCMS R_{T}=1.370 min, m/z=605.2 [M+H]⁺.

### Step 2: (S)-tert-butyl 3-(6-cyano-8-(2-(hydroxymethyl)thieno[3,2-b]pyridin-7-yl)-3,4-dihydroquinolin-1(2H)-yl)pyrrolidine-1-carboxylate

To a solution of (S)-tert-butyl 3-(8-(2-(((tert-butyldimethylsilyl)oxy)methyl)thieno[3,2-b]pyridin-7-yl)-6-cyano-3,4-dihydroquinolin-1(2H)-yl)pyrrolidine-1-carboxylate (200 mg, 0.33 mmol) in tetrahydrofuran (4 mL) was added tetrabutylammonium fluoride (1 M in tetrahydrofuran, 0.06 mL). After stirring at 20° C. for 30 min, the reaction mixture was purified by prep-TLC to afford the title compound. LCMS R_{T}=0.835 min, m/z=491.1 [M+H]⁺.

### Step 3

To a solution of (S)-tert-butyl 3-(6-cyano-8-(2-(hydroxymethyl)thieno[3,2-b]pyridin-7-yl)-3,4-dihydroquinolin-1(2H)-yl)pyrrolidine-1-carboxylate (20 mg, 0.04 mmol) in tetrahydrofuran (1 mL) were added succinimide (8 mg, 0.08 mmol), triphenylphosphine (21 mg, 0.08 mmol) and diisopropyl azodicarboxylate (16 mg, 0.08 mmol) at 0° C. After stirring at 50° C. for 1 h, the reaction was concentrated under reduced pressure. The residue was dissolved in hydrochloric acid (4 M in dioxane, 3 mL). After stirring at 20° C. for 30 min, the reaction was concentrated under reduced pressure. The residue was purified by RP-HPLC to afford Example 82. ¹H NMR (400 MHz, CD₃OD) δ=8.75 (s, 1H), 8.50 (s, 1H), 7.61-7.40 (m, 4H), 4.95 (s, 4H), 3.85 (s, 1H), 3.24-2.85 (m, 4H), 2.82-2.81 (m, 1H), 2.75 (s, 6H), 2.11-1.43 (m, 4H). LCMS R_{T}=0.615 min, m/z=472.1 [M+H]⁺.

### Example 83: (S)-8-(2-((2,6-dioxo-3-(2,2,2-trifluoroethyl)-2,3-dihydropyrimidin-1(6H)-yl)methyl)thieno[3,2-b]pyridin-7-yl)-1-(pyrrolidin-3-yl)-1,2,3,4-tetrahydroquinoline-6-carbonitrile, formic acid salt

Example 83 was prepared from 1-(2,2,2-trifluoroethyl)pyrimidine-2,4(1H,3H)-dione and (S)-tert-butyl 3-(6-cyano-8-(2-(hydroxymethyl)thieno[3,2-b]pyridin-7-yl)-3,4-dihydroquinolin-1(2H)-yl)pyrrolidine-1-carboxylate (Example 82, Step 2), following the procedure described in the synthesis of Example 82. The final compound was purified by RP-HPLC. ¹H NMR (400 MHz, CD30D) 6=8.77 (s, 1H), 8.51 (s, 1H), 7.67 (d, J=8.0 Hz, 1H), 7.61-7.47 (m, 4H), 5.89 (d, J=8.0 Hz, 1H), 5.41 (s, 2H), 4.64 (q, J=8.4 Hz, 3H), 3.87 (s, 1H), 3.31-2.96 (m, 3H), 3.00-2.38 (m, 4H), 2.11-1.40 (m, 4H). LCMS R_{T}=1.194 min, m/z=567.0 [M+H]⁺.

### Example 84: (S)-8-(2-((2,6-dioxo-3-(2,2,2-trifluoroethyl)-2,3-dihydropyrimidin-1(6H)-yl)methyl)thieno[3,2-b]pyridin-7-yl)-1-(1-ethylpyrrolidin-3-yl)-1,2,3,4-tetrahydroquinoline-6-carbonitrile

To a solution of (S)-8-(2-((2,6-dioxo-3-(2,2,2-trifluoroethyl)-2,3-dihydropyrimidin-1(6H)-yl)methyl)thieno[3,2-b]pyridin-7-yl)-1-(pyrrolidin-3-yl)-1,2,3,4-tetrahydroquinoline-6-carbonitrile (Example 83, 100 mg, 0.88 mmol) in acetonitrile (2 mL) were added sodium triacetoxyborohydride (56 mg, 0.26 mmol), triethylamine (26 mg, 0.26 mmol) and acetaldehyde (46 mg, 0.1 mmol). After stirring at 25° C. for 1 h, the reaction was concentrated under reduced pressure. The residue was purified by RP-HPLC to afford the title compound. ¹H NMR (400 MHz, CD₃OD) δ=8.80 (d, J=4.8 Hz, 1H), 7.44 (d, J=8.0 Hz, 1H), 7.54 (s, 1H), 7.50-7.41 (m, 2H), 7.40 (s, 1H), 5.86 (d, J=8.0 Hz, 1H), 5.38 (s, 2H), 4.69-4.54 (m, 3H), 3.78-3.62 (m, 1H), 2.84 (s, 2H), 2.50-1.91 (m, 6H), 1.66 (s, 2H), 1.63-0.27 (m, 6H). LCMS R_{T}=0.966 min, m/z=595.4 [M+H]⁺

### Example 85: 8-(2-((2,6-dioxo-3-(2,2,2-trifluoroethyl)-2,3-dihydropyrimidin-1(6H)-yl)methyl)thieno[3,2-b]pyridin-7-yl)-1-((S)-1-((1s,3R)-3-methoxycyclobutyl)pyrrolidin-3-yl)-1,2,3,4-tetrahydroquinoline-6-carbonitrile

Example 85 was prepared from (S)-8-(2-((2,6-dioxo-3-(2,2,2-trifluoroethyl)-2,3-dihydropyrimidin-1(6H)-yl)methyl)thieno[3,2-b]pyridin-7-yl)-1-(pyrrolidin-3-yl)-1,2,3,4-tetrahydroquinoline-6-carbonitrile (Example 83) and 3-methoxycyclobutanone, following the procedure described in the synthesis of Example 84. The final compound was purified by RP-HPLC. ¹H NMR (400 MHz, CD₃OD) δ=8.72 (d, J=4.8 Hz, 1H), 8.46 (s, 1H), 7.66 (d, J=7.6 Hz, 1H), 7.56 (s, 1H), 7.48 (s, 2H), 7.42 (d, J=1.6 Hz, 1H), 5.88 (d, J=8.0 Hz, 1H), 5.38 (d, J=8.4 Hz, 2H), 4.62 (q, J=8.4 Hz, 3H), 3.94-3.44 (m, 2H), 3.21-3.12 (m, 3H), 2.88 (d, J=5.2 Hz, 3H), 2.62-2.09 (m, 5H), 2.06-1.79 (m, 4H), 1.72-0.33 (m, 4H). LCMS R_{T}=1.538 min, m/z=651.2 [M+H]⁺

### Example 86: (S)-1-(1-cyclobutylpyrrolidin-3-yl)-8-(2-((2,6-dioxo-3-(2,2,2-trifluoroethyl)-2,3-dihydropyrimidin-1(6H)-yl)methyl)thieno[3,2-b]pyridin-7-yl)-1,2,3,4-tetrahydroquinoline-6-carbonitrile

Example 86 was prepared from (S)-8-(2-((2,6-dioxo-3-(2,2,2-trifluoroethyl)-2,3-dihydropyrimidin-1(6H)-yl)methyl)thieno[3,2-b]pyridin-7-yl)-1-(pyrrolidin-3-yl)-1,2,3,4-tetrahydroquinoline-6-carbonitrile (Example 83) and cyclobutanone, following the procedure described in the synthesis of Example 84. The final compound was purified by RP-HPLC. ¹H NMR (400 MHz, CD₃OD) δ=8.74 (d, J=4.8 Hz, 1H), 7.66 (d, J=7.6 Hz, 1H), 7.58 (s, 1H), 7.54-7.44 (m, 2H), 7.42 (s, 1H), 5.88 (d, J=8.0 Hz, 1H), 5.50-5.25 (m, 2H), 4.64 (q, J=8.4 Hz, 3H), 3.66 (q, J=7.6 Hz, 1H), 2.88 (d, J=5.2 Hz, 2H), 2.76-2.47 (m, 1H), 2.40-1.45 (m, 13H), 1.43-0.31 (m, 2H). LCMS R_{T}=0.786 min, m/z=620.6 [M+H]⁺.

### Example 87: (S)-8-(2-((3-methyl-2,6-dioxo-2,3-dihydropyrimidin-1(6H)-yl)methyl)thieno[3,2-b]pyridin-7-yl)-1-(pyrrolidin-3-yl)-1,2,3,4-tetrahydroquinoline-6-carbonitrile, formic acid salt

Example 87 was prepared from 1-methylpyrimidine-2,4(1H,3H)-dione and (S)-tert-butyl 3-(6-cyano-8-(2-(hydroxymethyl)thieno[3,2-b]pyridin-7-yl)-3,4-dihydroquinolin-1(2H)-yl)pyrrolidine-1-carboxylate (Example 82, Step 2), following the procedure described in the synthesis of Example 84. The final compound was purified by RP-HPLC. ¹H NMR (400 MHz, CD3OD) δ=8.73 (d, J=3.6 Hz, 1H), 8.52 (s, 1H), 7.58 (d, J=8.0 Hz, 1H), 7.43-7.56 (m, 4H), 5.75 (d, J=8.0 Hz, 1H), 5.38 (s, 2H), 3.73-3.92 (m, 1H), 3.38 (s, 5H), 2.83-3.23 (m, 4H), 2.33-2.82 (m, 2H), 1.52-2.03 (m, 4H). LCMS R_{T}=1.377 min, m/z=498.9 [M+H]⁺.

### Example 88: (S)-8-(2-((3-cyclopropyl-2,6-dioxo-2,3-dihydropyrimidin-1(6H)-yl)methyl)thieno[3,2-b]pyridin-7-yl)-1-(pyrrolidin-3-yl)-1,2,3,4-tetrahydroquinoline-6-carbonitrile, formic acid salt

Example 88 was prepared from 1-cyclopropylpyrimidine-2,4(1H,3H)-dione and (S)-tert-butyl 3-(6-cyano-8-(2-(hydroxymethyl)thieno[3,2-b]pyridin-7-yl)-3,4-dihydroquinolin-1(2H)-yl)pyrrolidine-1-carboxylate (Example 82, Step 2), following the procedure described in the synthesis of Example 84. The final compound was purified by RP-HPLC. ¹H NMR (400 MHz, CD₃OD) δ=8.74 (s, 1H), 8.53 (s, 1H), 7.61 (d, J=8.0 Hz, 1H), 7.51 (dd, J=13.2, 11.6 Hz, 4H), 5.73 (d, J=8.0 Hz, 1H), 5.37 (s, 2H), 3.84 (s, 1H), 3.31-2.47 (m, 9H), 2.10-1.45 (m, 4H), 1.09-0.98 (m, 2H), 0.91-0.81 (m, 2H) LCMS RT=0.878 min, m/z=525.1 [M+H]⁺.

### Example 89: (S)-8-(2-((3-(2,2-difluoropropyl)-2,6-dioxo-2,3-dihydropyrimidin-1(6H)-yl)methyl)thieno[3,2-b]pyridin-7-yl)-1-(pyrrolidin-3-yl)-1,2,3,4-tetrahydroquinoline-6-carbonitrile, formic acid salt

Example 89 was prepared from 1-(2,2-difluoropropyl)pyrimidine-2,4(1H,3H)-dione and (S)-tert-butyl 3-(6-cyano-8-(2-(hydroxymethyl)thieno[3,2-b]pyridin-7-yl)-3,4-dihydroquinolin-1(2H)-yl)pyrrolidine-1-carboxylate (Example 82, Step 2), following the procedure described in the synthesis of Example 84. The final compound was purified by RP-HPLC. ¹H NMR (400 MHz, MeOD) δ=8.74 (s, 1H), 8.52 (s, 1H), 7.46-7.62 (m, 5H), 5.82 (d, J=7.6 Hz, 1H), 5.38 (s, 2H), 4.28 (m, J=13.6, 2.8 Hz, 2H), 3.84 (s, 1H), 2.38-3.30 (m, 8H), 1.98 (s, 3H), 1.66 (t, J=18.8 Hz, 3H), 1.54 (s, 1H). LCMS R_{T}=0.669 min, m/z=563.1 [M+H]⁺.

### Example 90: (R)-1-(2,2-difluoroethyl)-3-((7-(1-(pyrrolidin-3-yl)-6-(trifluoromethyl)-1,2,3,4-tetrahydroquinolin-8-yl)thieno[3,2-b]pyridin-2-yl)methyl)pyrimidine-2,4(1H,3H)-dione

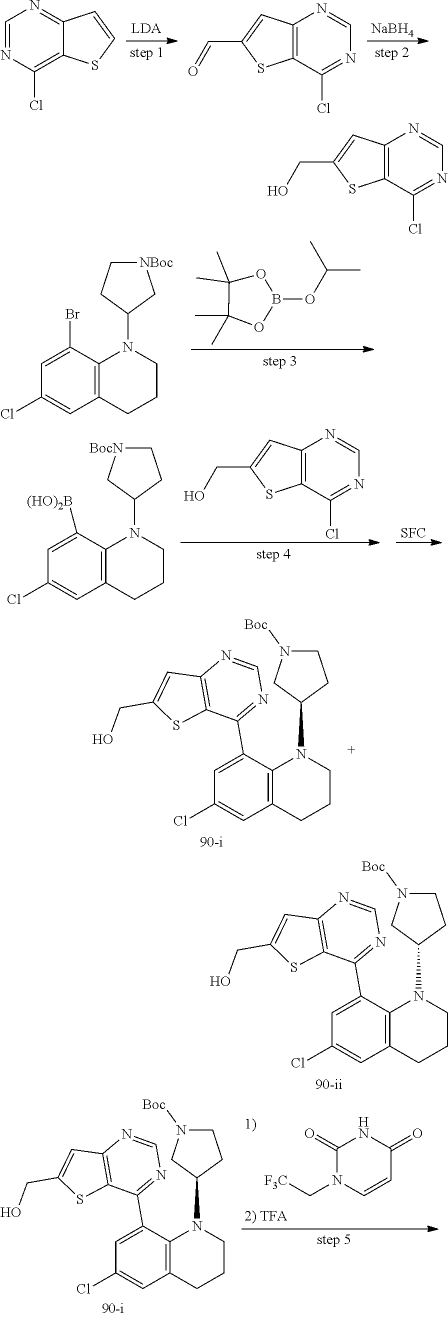

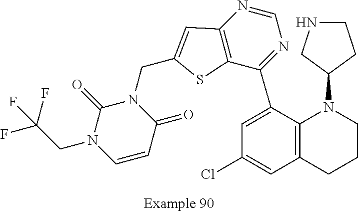 [SMILES: O=c1ccn(CC(F)(F)F)c(=O)n1Cc1cc2ncnc(-c3cc(Cl)cc4c3N([C@@H]3CCNC3)CCC4)c2s1]

### Step 1: 4-chlorothieno[3,2-d]pyrimidine-6-carbaldehyde

To a solution of 4-chlorothieno[3,2-d]pyrimidine (1.1 g, 6.45 mmol) in tetrahydrofuran (25 mL) was added lithium diisopropylamide (2 M in tetrahydrofuran, 4.19 mL) slowly at −63° C. After stirring at −45° C. for 30 min, N,N-dimethylformamide (942 mg, 12.89 mmol) was added at −63° C. After stirring at −63° C. for 1 h, the pH of the reaction mixture was adjusted 6 by addition of aqueous hydrochloric acid (1 M, 28 mL). Then the mixture was concentrated under reduced pressure. The mixture was diluted with water (50 mL) and extracted with ethyl acetate (40 mL×3). The combined organic layers were washed with brine (50 mL), dried over anhydrous sodium sulfate, filtered and concentrated under reduced pressure. The residue was purified by column chromatography to afford the title compound. ¹H NMR (400 MHz, CD₃OD) δ=8.96-8.87 (m, 1H), 7.60-7.53 (m, 1H), 5.95-5.86 (m, 1H).

### Step 2: (4-chlorothieno[3,2-d]pyrimidin-6-yl)methanol

To a solution of 4-chlorothieno[3,2-d]pyrimidine-6-carbaldehyde (670 mg, 3.37 mmol) in methanol (10 mL) was added sodium borohydride (383 mg, 10.12 mmol) at 0° C. After stirring at 25° C. for 2 h, the reaction was quenched by addition of saturated aqueous ammonium chloride (10 mL). The mixture was concentrated under reduced pressure, diluted with water (20 mL) and extracted with ethyl acetate (15 mL×2). The combined organic layers were washed with brine (15 mL), dried over anhydrous sodium sulfate, filtered and concentrated under reduced pressure. The residue was purified by column chromatography to afford the title compound.

### Step 3: (1-(1-(tert-butoxycarbonyl)pyrrolidin-3-yl)-6-chloro-1,2,3,4-tetrahydroquinolin-8-yl)boronic acid

To a solution of isopropyl magnesium chloride lithium chloride complex (1.3 M in tetrahydrofuran, 13.88 mL) in tetrahydrofuran (8 mL) was added tert-butyl 3-(8-bromo-6-chloro-3,4-dihydroquinolin-1(2H)-yl)pyrrolidine-1-carboxylate (1.5 g, 3.61 mmol) in tetrahydrofuran (6 mL) at 0° C. After stirring at 0° C. for 0.5 h, 2-isopropoxy-4,4,5,5-tetramethyl-1,3,2-dioxaborolane (2.0 g, 10.82 mmol) was added and the mixture was stirred at 20° C. for 0.5 h. The reaction was quenched by addition of water (10 mL) and concentrated under reduced pressure. The residue was mixed with ethyl acetate (30 mL) and water (30 mL). The layers were separated and the organic phase was dried over anhydrous sodium sulfate, filtered and concentrated under reduced pressure to afford the title compound. It was used in the next step without further purification.

### Step 4

To a solution of (4-chlorothieno[3,2-d]pyrimidin-6-yl)methanol (500 mg, 2.49 mmol) and (1-(1-(tert-butoxycarbonyl)pyrrolidin-3-yl)-6-chloro-1,2,3,4-tetrahydroquinolin-8-yl)boronic acid (1.5 g, 3.99 mmol) in dioxane (25 mL) and water (1.5 mL) were added potassium carbonate (861 mg, 6.23 mmol) and [1,1′-bis(diphenylphosphino)ferrocene]dichloropalladium(II) (364 mg, 0.5 mmol). After stirring at 100° C. for 2 h, the reaction was filtered and concentrated under reduced pressure. The residue was purified by column chromatography to give (±)-tert-butyl 3-(6-chloro-8-(6-(hydroxymethyl)thieno[3,2-d]pyrimidin-4-yl)-3,4-dihydroquinolin-1(2H)-yl)pyrrolidine-1-carboxylate. The racemate was separated by SFC to give first eluting fraction (90-i, Rt=2.088 min) and second eluting fraction (90-ii, Rt=2.340 min).

### Step 5: (R)-3-((4-(6-chloro-1-(pyrrolidin-3-yl)-1,2,3,4-tetrahydroquinolin-8-yl)thieno[3,2-d]pyrimidin-6-yl)methyl)-1-(2,2,2-trifluoroethyl)pyrimidine-2,4(1H,3H)-dione

To a solution of 90-i (70 mg, 0.14 mmol), 1-(2,2,2-trifluoroethyl)pyrimidine-2,4(1H,3H)-dione (54 mg, 0.28 mmol) and 1,1′-azobis(N,N-dimethylformamide) (48 mg, 0.28 mmol) in toluene (2.5 mL) was added tributylphosphine (113 mg, 0.56 mmol). After stirring at 100° C. for 1 h, the reaction was concentrated to remove the solvent. Then the residue was dissolved in dichloromethane (4 mL) and trifluoroacetic acid (0.82 mL). After stirring at 20° C. for 1 h, the reaction was concentrated under reduced pressure. The residue was purified by RP-HPLC to afford the title compound. ¹H NMR (400 MHz, CD₃OD) δ=9.18 (s, 1H), 7.65 (d, J=8.0 Hz, 1H), 7.52 (s, 1H), 7.31 (d, J=2.4 Hz, 1H), 7.17 (d, J=2.4 Hz, 1H), 5.88 (d, J=8.0 Hz, 1H), 5.43 (s, 2H), 4.66-4.58 (m, 2H), 3.43 (q, J=8.0 Hz, 1H), 3.25-3.16 (m, 2H), 2.84 (t, J=6.3 Hz, 2H), 2.73 (s, 1H), 2.51-2.39 (m, 2H), 2.13-1.87 (m, 3H), 1.52 (s, 1H), 1.18-1.16 (m, 1H), 1.18-0.86 (m, 1H). LCMS R_{T}=1.060 min, m/z=577.3 [M+H]⁺. The absolute configuration was tentatively assigned based on the relative potency of the pair enantiomers in biology assays.

### Example 91: (S)-3-((4-(6-chloro-1-(pyrrolidin-3-yl)-1,2,3,4-tetrahydroquinolin-8-yl)thieno[3,2-d]pyrimidin-6-yl)methyl)-1-(2,2,2-trifluoroethyl)pyrimidine-2,4(1H,3H)-dione

Example 91 was prepared 90-ii and 1-(2,2,2-trifluoroethyl)pyrimidine-2,4(1H,3H)-dione, following the procedure described in the synthesis of Example 90. The final compound was purified by RP-HPLC. ¹H NMR (400 MHz, CD₃OD) δ=9.18 (s, 1H), 7.65 (d, J=8.0 Hz, 1H), 7.52 (s, 1H), 7.31 (d, J=2.4 Hz, 1H), 7.17 (d, J=2.4 Hz, 1H), 5.88 (d, J=8.0 Hz, 1H), 5.43 (s, 2H), 4.68-4.59 (m, 2H), 3.49-3.37 (m, 1H), 3.27-3.16 (m, 2H), 2.84 (t, J=6.0 Hz, 2H), 2.77-1.74 (m, 6H), 1.67-0.67 (m, 2H). LCMS R_{T}=1.047 min, m/z=577.3 [M+H]⁺.

### Example 92: (S)-8-(6-((2,6-dioxo-3-(2,2,2-trifluoroethyl)-2,3-dihydropyrimidin-1(6H)-yl)methyl)thieno[3,2-d]pyrimidin-4-yl)-1-(pyrrolidin-3-yl)-1,2,3,4-tetrahydroquinoline-6-carbonitrile, formic acid salt

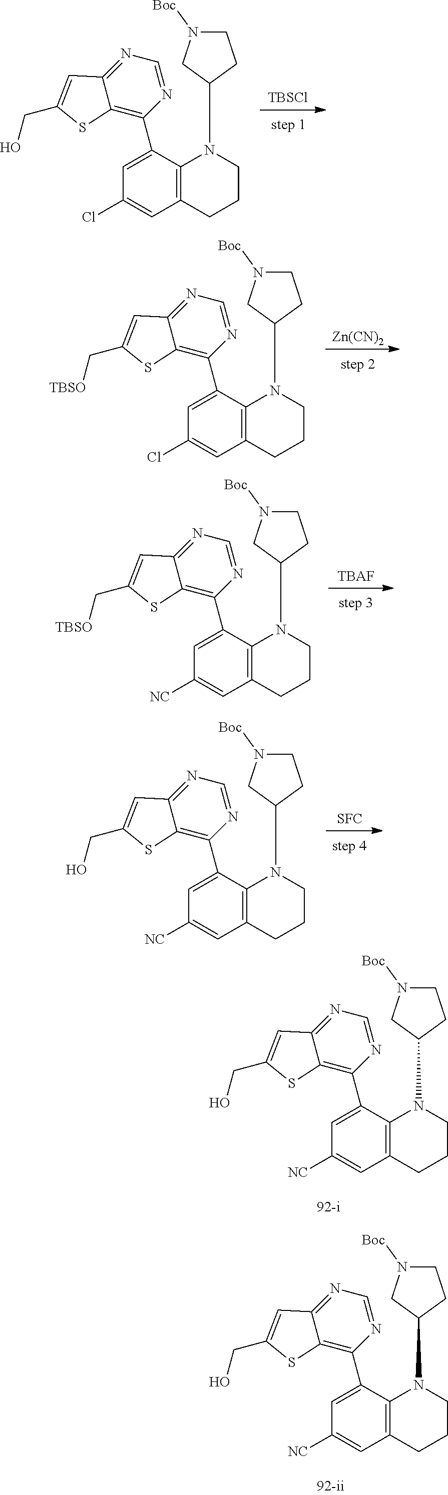

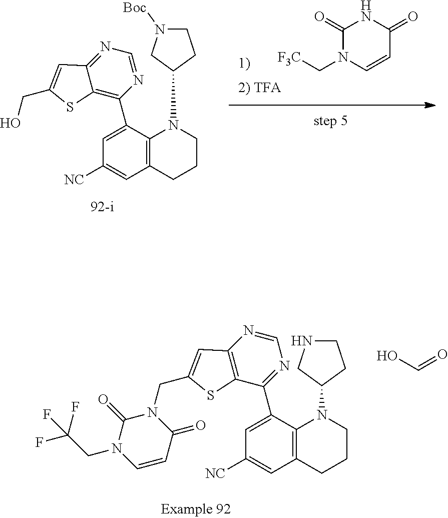 [SMILES: CC(C)(C)OC(=O)N1CC[C@H](N2CCCc3cc(C#N)cc(-c4ncnc5cc(CO)sc45)c32)C1 ; O=c1ccn(CC(F)(F)F)c(=O)[nH]1 ; N#Cc1cc2c(c(-c3ncnc4cc(Cn5c(=O)ccn(CC(F)(F)F)c5=O)sc34)c1)N([C@H]1CCNC1)CCC2 ; O=CO]

### Step 1: (f)-tert-butyl 3-(8-(6-(((tert-butyldimethylsilyl)oxy)methyl)thieno[3,2-d]pyrimidin-4-yl)-6-chloro-3,4-dihydroquinolin-1(2H)-yl)pyrrolidine-1-carboxylate

To a solution of (±)-tert-butyl 3-(6-chloro-8-(6-(hydroxymethyl)thieno[3,2-d]pyrimidin-4-yl)-3,4-dihydroquinolin-1(2H)-yl)pyrrolidine-1-carboxylate (400 mg, 0.8 mmol) in dichloromethane (10 mL) were added imidazole (163 mg, 2.4 mmol) and tert-butylchlorodimethylsilane (240 mg, 1.6 mmol). After stirring at 40° C. for 15 h, the reaction was diluted with water (20 mL) and extracted with dichloromethane (15 mL×3). The combined organic layers were washed with brine (25 mL), dried over anhydrous sodium sulfate, filtered and concentrated under reduced pressure. The residue was purified by column chromatography afford (450 mg, 92%). LCMS R_{T}=1.300 min, m/z=616.9 [M+H]⁺.

### Step 2: (f)-tert-butyl 3-(8-(6-(((tert-butyldimethylsilyl)oxy)methyl)thieno[3,2-d]pyrimidin-4-yl)-6-cyano-3,4-dihydroquinolin-1(2H)-yl)pyrrolidine-1-carboxylate

To a solution of (±)-tert-butyl 3-(8-(6-(((tert-butyldimethylsilyl)oxy)methyl)thieno[3,2-d]pyrimidin-4-yl)-6-chloro-3,4-dihydroquinolin-1(2H)-yl)pyrrolidine-1-carboxylate (450 mg, 0.73 mmol) in dioxane (5 mL) were added diisopropylethylamine (236 mg, 1.83 mmol), zinc cyanide (111 mg, 0.95 mmol) and BrettPhos Pd G3 (74 mg, 0.15 mmol). After stirring at 90° C. for 16 h, the reaction was concentrated under reduced pressure. The residue was diluted with water (20 mL) and extracted with ethyl acetate (15 mL×3). The combined organic layers were washed with brine (25 mL), dried over anhydrous sodium sulfate, filtered and concentrated under reduced pressure to afford the crude title compound. It was used in next step without further purification. LCMS R_{T}=1.700 min, m/z=606.5 [M+H]⁺.

### Step 3: (f)-tert-butyl 3-(6-cyano-8-(6-(hydroxymethyl)thieno[3,2-d]pyrimidin-4-yl)-3,4-dihydroquinolin-1(2H)-yl)pyrrolidine-1-carboxylate

To a solution of (±)-tert-butyl 3-(8-(6-(((tert-butyldimethylsilyl)oxy)methyl)thieno[3,2-d]pyrimidin-4-yl)-6-cyano-3,4-dihydroquinolin-1(2H)-yl)pyrrolidine-1-carboxylate (400 mg, 0.66 mmol) in tetrahydrofuran (10 mL) was added tetrabutylammonium fluoride (1 M in tetrahydrofuran, 0.66 mL). After stirring at 25° C. for 1 h, the reaction was concentrated under reduced pressure. The residue was purified by column chromatography to afford the title compound. LCMS R_{T}=0.887 min, m/z=492.2 [M+H]⁺.

### Step 4: (S)-tert-butyl 3-(6-cyano-8-(6-(hydroxymethyl)thieno[3,2-d]pyrimidin-4-yl)-3,4-dihydroquinolin-1(2H)-yl)pyrrolidine-1-carboxylate and (R)-tert-butyl 3-(6-cyano-8-(6-(hydroxymethyl)thieno[3,2-d]pyrimidin-4-yl)-3,4-dihydroquinolin-1(2H)-yl)pyrrolidine-1-carboxylate

The racemate from Step 3 was separated by SFC to give first eluting fraction (92-i, Rₜ=3.320 min) and second eluting fraction (92-ii, Rₜ=3.462 min). LCMS R_{T}=1.843 min, m/z=492.2 [M+H]⁺.

### Step 5

To a solution of 92-I (45 mg, 0.09 mmol) in tetrahydrofuran (1.5 mL) was added 1-(2,2,2-trifluoroethyl)pyrimidine-2,4(1H,3H)-dione (26 mg, 0.14 mmol), triphenylphosphine (60 mg, 0.23 mmol) and diisopropyl azodicarboxylate (46 mg, 0.23 mmol). After stirring at 50° C. for 2 h, the reaction was concentrated under reduced pressure. The residue was dissolved in dichloromethane (5 mL) and added trifluoroacetic acid (1.54 g, 13.51 mmol). After stirring at 20° C. for 1 h, the reaction was concentrated under reduced pressure. The residue was purified by RP-HPLC to afford Example 92. ¹H NMR (400 MHz, CD₃OD) δ=9.26 (s, 1H), 8.51 (s, 1H), 7.67 (d, J=8.0 Hz, 2H), 7.58-7.48 (m, 2H), 5.90 (d, J=8.0 Hz, 1H), 5.44 (s, 2H), 3.73 (m, J=8.0 Hz, 1H), 3.39-3.32 (m, 2H), 3.17-2.61 (m, 6H), 2.31-0.89 (m, 6H). LCMS R_{T}=1.208 min, m/z=568.3 [M+H]⁺. The absolute configuration was tentatively assigned based on the relative potency of the pair enantiomers in biology assays.

### Example 93: (R)-8-(6-((2,6-dioxo-3-(2,2,2-trifluoroethyl)-2,3-dihydropyrimidin-1(6H)-yl)methyl)thieno[3,2-d]pyrimidin-4-yl)-1-(pyrrolidin-3-yl)-1,2,3,4-tetrahydroquinoline-6-carbonitrile, formic acid salt

Example 93 was prepared from 92-ii and 1-(2,2,2-trifluoroethyl)pyrimidine-2,4(1H,3H)-dione, following the procedure described in the synthesis of Example 92. The final compound was purified by RP-HPLC. ¹H NMR (400 MHz, CD₃OD) δ=9.27-9.23 (m, 1H), 8.51-8.47 (m, 1H), 7.69-7.65 (m, 2H), 7.58-7.50 (m, 2H), 5.91-5.87 (m, 1H), 5.45-5.42 (m, 2H), 4.66-4.59 (m, 2H), 3.81-3.70 (m, 1H), 3.15-3.15 (m, 1H), 3.21-3.10 (m, 1H), 2.96-2.68 (m, 4H), 2.32-1.16 (m, 6H). LCMS RT=1.241 min, m/z=568.3 [M+H]⁺. The absolute configuration was tentatively assigned based on the relative potency of the pair enantiomers in biology assays.

### Example 94: 1-((7-(6-chloro-1-((3R,5R)-5-(hydroxymethyl)pyrrolidin-3-yl)-1,2,3,4-tetrahydroquinolin-8-yl)thieno[3,2-b]pyridin-2-yl)methyl)pyrrolidine-2,5-dione, formic acid salt

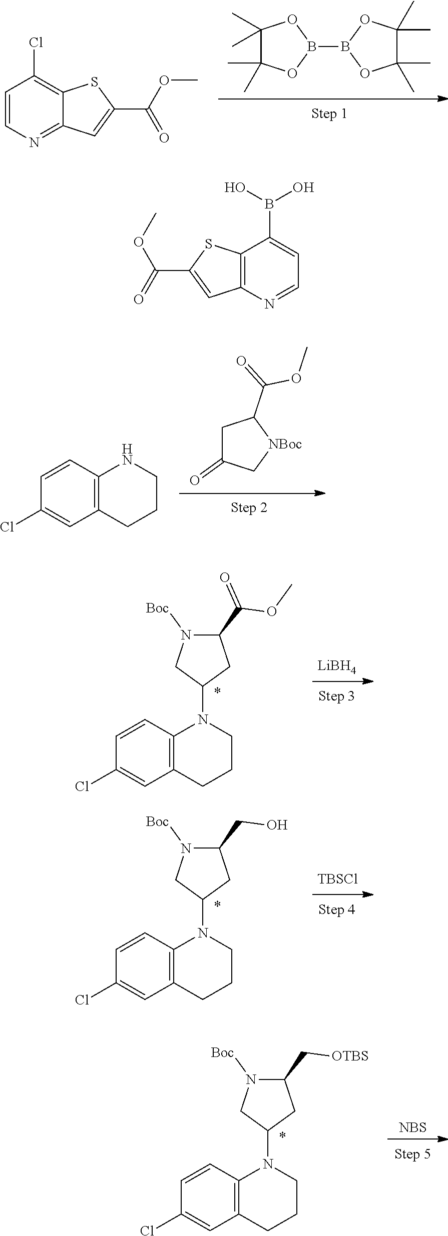

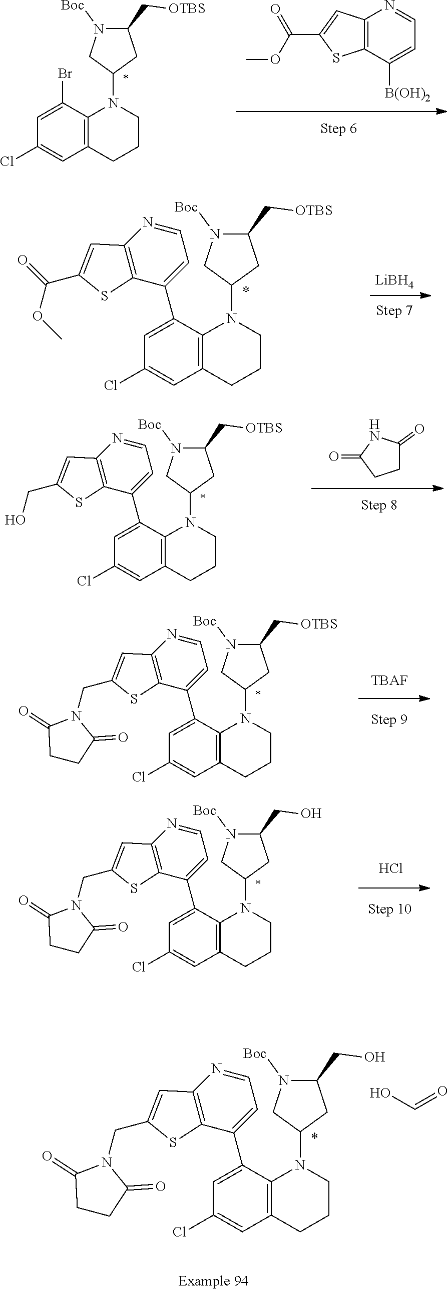

### Step 1: (2-(methoxycarbonyl)thieno[3,2-b]pyridin-7-yl)boronic acid

To a mixture of methyl 7-chlorothieno[3,2-b]pyridine-2-carboxylate (200 mg, 0.70 mmol), bis(pinacolato)diboron (357 mg, 1.41 mmol) and potassium acetate (207 mg, 2.11 mmol) in dioxane (3 mL) was added dichloro[1,1′-bis(diphenylphosphino)ferrocene]palladium(II) (103 mg, 0.14 mmol) under nitrogen atmosphere. After stirring at 110° C. for 2 h, the reaction was filtered and concentrated under reduced pressure to give crude (2-(methoxycarbonyl)thieno[3,2-b]pyridin-7-yl)boronic acid. It was used in next step without further purification. LCMS R_{T}=0.435, m/z=238.1 [M+H]⁺.

### Step 2: (2R)-1-tert-butyl 2-methyl 4-(6-chloro-3,4-dihydroquinolin-1(2H)-yl)pyrrolidine-1,2-dicarboxylate

A mixture of 6-chloro-1,2,3,4-tetrahydroquinoline (473 mg, 2.26 mmol), (R)-1-tert-butyl 2-methyl 4-oxopyrrolidine-1,2-dicarboxylate (500 mg, 2.06 mmol) and sodium triacetoxyborohydride (1.3 g, 6.17 mmol) in acetonitrile (6 mL) and trifluoroacetic acid (351 mg, 3.08 mmol) was stirred at 25° C. for 6 h under nitrogen atmosphere. The reaction was quenched by addition of saturated aqueous sodium bicarbonate (3 mL) and water (40 mL) and extracted with ethyl acetate (30 mL×3). The combined organic layers were washed with brine (10 mL), dried over anhydrous sodium sulfate, filtered and concentrated under reduced pressure. The residue was purified by column chromatography to afford the title compound. LCMS R_{T}=1.080 min, m/z=395.1 [M+H]⁺.

### Step 3: (2R)-tert-butyl 4-(6-chloro-3,4-dihydroquinolin-1(2H)-yl)-2-(hydroxymethyl)pyrrolidine-1-carboxylate

To a mixture of lithium borohydride (33 mg, 1.52 mmol) in tetrahydrofuran (1.5 mL) was added a solution of (2R)-1-tert-butyl 2-methyl 4-(6-chloro-3,4-dihydroquinolin-1(2H)-yl)pyrrolidine-1,2-dicarboxylate (300 mg, 0.76 mmol) in tetrahydrofuran (1 mL) at −78° C. under nitrogen atmosphere. After stirring at 25° C. for 2 h, the reaction was quenched by addition of aqueous ammonium chloride solution (10 mL) and extracted with ethyl acetate (15 mL×3). The combined organic layers were washed with brine (30 mL), dried over anhydrous sodium sulfate, filtered and concentrated under reduced pressure to afford the crude title compound. It was used in next step without further purification. LCMS R_{T}=1.115 min, m/z=367.2 [M+H]⁺.

### Step 4: (2R)-tert-butyl 2-(((tert-butyldimethylsilyl)oxy)methyl)-4-(6-chloro-3,4-dihydroquinolin-1(2H)-yl)pyrrolidine-1-carboxylate

A mixture of (2R)-tert-butyl 4-(6-chloro-3,4-dihydroquinolin-1(2H)-yl)-2-(hydroxymethyl)pyrrolidine-1-carboxylate (260 mg, 0.71 mmol), imidazole (120 mg, 1.77 mmol) and tert-butylchlorodimethylsilane (139 mg, 0.92 mmol) in dichloromethane (7 mL) was stirred at 25° C. for 2 h. The reaction was washed with brine (5 mL×3), dried over anhydrous sodium sulfate, filtered and concentrated under reduced pressure. The residue was purified by column chromatography to afford the title compound. LCMS R_{T}=1.432 min, m/z=481.2 [M+H]⁺.

### Step 5: (2R)-tert-butyl 4-(8-bromo-6-chloro-3,4-dihydroquinolin-1(2H)-yl)-2-(((tert-butyldimethylsilyl)oxy)methyl)pyrrolidine-1-carboxylate

To a solution of (2R)-tert-butyl 2-(((tert-butyldimethylsilyl)oxy)methyl)-4-(6-chloro-3,4-dihydroquinolin-1(2H)-yl)pyrrolidine-1-carboxylate (450 mg, 0.94 mmol) in dichloromethane (10 mL) was added N-bromosuccinimide (183 mg, 1.03 mmol) at 0° C. After stirring at 25° C. for 0.5 h, the reaction was washed with brine (6 mL×3), dried over anhydrous sodium sulfate, filtered and concentrated under reduced pressure. The residue was purified by column chromatography to afford the title compound. LCMS R_{T}=1.263 min, m/z=561.1 [M+H]⁺.

### Step 6: methyl 7-(1-((5R)-1-(tert-butoxycarbonyl)-5-(((tert-butyldimethylsilyl)oxy)methyl)pyrrolidin-3-yl)-6-chloro-1,2,3,4-tetrahydroquinolin-8-yl)thieno[3,2-b]pyridine-2-carboxylate

To a mixture of (2R)-tert-butyl 4-(8-bromo-6-chloro-3,4-dihydroquinolin-1(2H)-yl)-2-(((tert-butyldimethylsilyl)oxy)methyl)pyrrolidine-1-carboxylate (260 mg, 0.46 mmol), (2-(methoxycarbonyl)thieno[3,2-b]pyridin-7-yl)boronic acid (165 mg, 0.7 mmol) and cesium carbonate (302 mg, 0.93 mmol) in dioxane (7 mL) and water (0.4 mL) was added dichloro[1,1′-bis(diphenylphosphino)ferrocene]palladium(II) (68 mg, 0.093 mmol) under nitrogen atmosphere. After stirring at 110° C. for 8 h, the reaction was filtered and concentrated under reduced pressure. The residue was purified by column chromatography to afford the title compound. LCMS R_{T}=1.587, m/z=672.4 [M+H]⁺.

### Step 7: (2R)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)pyrrolidine-1-carboxylate

To a mixture of lithium borohydride (10 mg, 0.46 mmol) in tetrahydrofuran (1 mL) was added methyl 7-(1-((5R)-1-(tert-butoxycarbonyl)-5-(((tert-butyldimethylsilyl)oxy)methyl)pyrrolidin-3-yl)-6-chloro-1,2,3,4-tetrahydroquinolin-8-yl)thieno[3,2-b]pyridine-2-carboxylate (140 mg, 0.21 mmol) in tetrahydrofuran (1 mL) at −78° C. under nitrogen atmosphere. After stirring at 25° C. for 2 h, the reaction was quenched by addition of aqueous ammonium chloride solution (2 mL) and extracted with ethyl acetate (5 mL×3). The combined organic layers were washed with brine (10 mL), dried over anhydrous sodium sulfate, filtered and concentrated under reduced pressure. The residue was purified by prep-TLC to afford the title compound. LCMS R_{T}=1.230 min, m/z=644.4 [M+H]⁺.

### Step 8: (2R)-tert-butyl 2-(((tert-butyldimethylsilyl)oxy)methyl)-4-(6-chloro-8-(2-((2,5-dioxopyrrolidin-1-yl)methyl)thieno[3,2-b]pyridin-7-yl)-3,4-dihydroquinolin-1(2H)-yl)pyrrolidine-1-carboxylate

To a mixture of (2R)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)pyrrolidine-1-carboxylate (30 mg, 0.047 mmol), succinimide (14 mg, 0.14 mmol) and triphenylphosphine (36 mg, 0.14 mmol) in tetrahydrofuran (0.5 mL) was added diisopropyl azodicarboxylate (28 mg, 0.14 mmol) under nitrogen atmosphere at 0° C. After stirring at 45° C. for 2 h, the reaction mixture was purified by prep-TLC to afford the title compound. LCMS R_{T}=1.583 min, m/z=725.5 [M+H]⁺.

### Step 9: (2R)-tert-butyl 4-(6-chloro-8-(2-((2,5-dioxopyrrolidin-1-yl)methyl)thieno[3,2-b]pyridin-7-yl)-3,4-dihydroquinolin-1(2H)-yl)-2-(hydroxymethyl)pyrrolidine-1-carboxylate

To a mixture of (2R)-tert-butyl 2-(((tert-butyldimethylsilyl)oxy)methyl)-4-(6-chloro-8-(2-((2,5-dioxopyrrolidin-1-yl)methyl)thieno[3,2-b]pyridin-7-yl)-3,4-dihydroquinolin-1(2H)-yl)pyrrolidine-1-carboxylate (33 mg, 0.045 mmol) in tetrahydrofuran (2 mL) was added tetrabutylammonium fluoride (1 M in tetrahydrofuran, 0.046 mL). After stirring at 25° C. for 2 h, the reaction mixture was purified by prep-TLC to afford the title compound. LCMS R_{T}=1.213 min, m/z=611.4 [M+H]⁺.

### Step 10

(2R)-tert-butyl 4-(6-chloro-8-(2-((2,5-dioxopyrrolidin-1-yl)methyl)thieno[3,2-b]pyridin-7-yl)-3,4-dihydroquinolin-1(2H)-yl)-2-(hydroxymethyl)pyrrolidine-1-carboxylate (25 mg, 0.041 mmol) was dissolved in hydrochloric acid (4 M in dioxane, 0.5 mL). After stirring at 25° C. for 30 min, the reaction was concentrated under reduced pressure. The residue was purified by RP-HPLC to afford Example 94. ¹H NMR (400 MHz, CD₃OD) δ=8.72 (d, J=4.8 Hz, 1H), 8.52 (s, 1H), 7.51-7.45 (m, 2H), 7.18 (d, J=2.4 Hz, 1H), 7.16 (s, 1H), 4.95 (s, 2H), 3.77-3.59 (m, 2H), 3.48 (s, 1H), 3.32 (s, 4H), 3.28-3.15 (m, 2H), 3.14-3.01 (m, 1H), 2.89 (t, J=6.4 Hz, 2H), 2.75 (s, 4H), 2.72-2.63 (m, 1H), 1.99-1.87 (m, 1H), 1.72 (s, 1H). LCMS R_{T}=0.979 min, m/z=511.3 [M+H]⁺.

### Example 95a and 95b: 3-((7-(6-chloro-1-((3S,5R)-5-methylpyrrolidin-3-yl)-1,2,3,4-tetrahydroquinolin-8-yl)thieno[3,2-b]pyridin-2-yl)methyl)-1-(2,2,2-trifluoroethyl)pyrimidine-2,4(1H,3H)-dione and 3-((7-(6-chloro-1-((3R,5S)-5-methylpyrrolidin-3-yl)-1,2,3,4-tetrahydroquinolin-8-yl)thieno[3,2-b]pyridin-2-yl)methyl)-1-(2,2,2-trifluoroethyl)pyrimidine-2,4(1H,3H)-dione

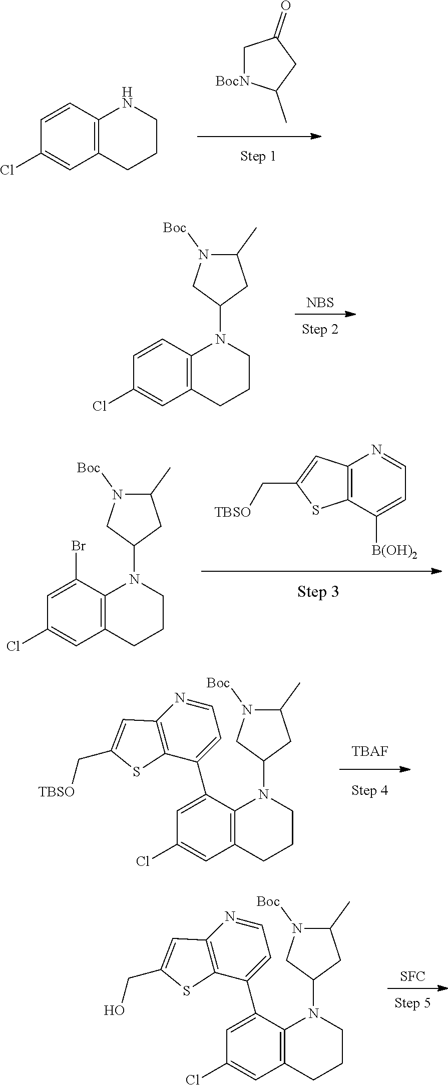

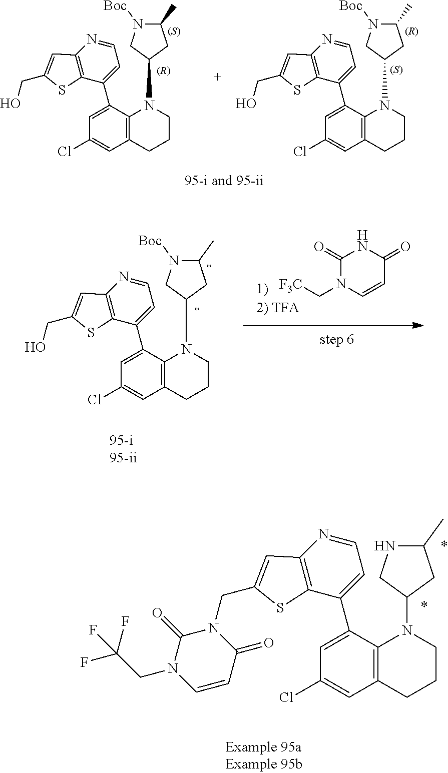

### Step 1: tert-butyl 4-(6-chloro-3,4-dihydro-2H-quinolin-1-yl)-2-methyl-pyrrolidine-1-carboxylate

To a solution of tert-butyl 2-methyl-4-oxo-pyrrolidine-1-carboxylate (500 mg, 2.51 mmol) in acetic acid (30 mL) were added 6-chloro-1,2,3,4-tetrahydroquinoline (617 mg, 2.76 mmol) and sodium triacetoxyborohydride (1.6 g, 7.53 mmol) at 20° C. After stirring at 20° C. for 12 h, the reaction mixture was diluted with saturated aqueous sodium hydrogen carbonate (20 mL) and extracted with ethyl acetate (20 mL×4). The combined organic layers were washed with brine (20 mL), dried over anhydrous sodium sulfate, filtered and concentrated under reduced pressure. The residue was purified by column chromatography to afford the title compound. LCMS R_{T}=1.153 min, m/z=351.0 [M+H]⁺.

### Step 2: tert-butyl 4-(8-bromo-6-chloro-3,4-dihydro-2H-quinolin-1-yl)-2-methyl-pyrrolidine-1-carboxylate

To a solution of tert-butyl 4-(6-chloro-3,4-dihydro-2H-quinolin-1-yl)-2-methyl-pyrrolidine-1-carboxylate (900 mg, 1.28 mmol) in dichloromethane (5 mL) was added N-bromosuccinimide (274 mg, 1.54 mmol) in dichloromethane (5 mL) at 0° C. After stirring at 0° C. for 1 h, the reaction mixture was concentrated under reduced pressure to remove solvent. The residue was purified by column chromatography to afford the title compound. LCMS R_{T}=1.213 min, m/z=431.0 [M+H]⁺.

### Step 3: tert-butyl 4-(8-(2-(((tert-butyldimethylsilyl)oxy)methyl)thieno[3,2-b]pyridin-7-yl)-6-chloro-3,4-dihydroquinolin-1(2H)-yl)-2-methylpyrrolidine-1-carboxylate

To a solution of tert-butyl 4-(8-bromo-6-chloro-3,4-dihydroquinolin-1(2H)-yl)-2-methylpyrrolidine-1-carboxylate (485 mg, 1.13 mmol) in dioxane (10 mL) and water (1 mL) were added (2-(((tert-butyldimethylsilyl)oxy)methyl)thieno[3,2-b]pyridin-7-yl)boronic acid (547 mg, 1.69 mmol), dichloro[1,1′-bis(diphenylphosphino)ferrocene]palladium(II) (247 mg, 0.34 mmol) and potassium carbonate (467 mg, 3.39 mmol) under nitrogen atmosphere. After stirring at 110° C. for 3 h, the reaction was concentrated under reduced pressure. The residue was purified by column chromatography to afford the title compound. LCMS R_{T}=0.917 min, m/z=627.2 [M+H]⁺.

### Step 4: (f)-tert-butyl 4-(6-chloro-8-(2-(hydroxymethyl)thieno[3,2-b]pyridin-7-yl)-3,4-dihydroquinolin-1(2H)-yl)-2-methylpyrrolidine-1-carboxylate

To a solution of tert-butyl 4-(8-(2-(((tert-butyldimethylsilyl)oxy)methyl)thieno[3,2-b]pyridin-7-yl)-6-chloro-3,4-dihydroquinolin-1(2H)-yl)-2-methylpyrrolidine-1-carboxylate (540 mg, 0.86 mmol) in tetrahydrofuran (10 mL) was added tetrabutylmmonium fluoride (1 M in tetrahydrofuran, 1.72 mL). After stirring at 25° C. for 0.5 h, the reaction was concentrated under reduced pressure. The residue was purified by column chromatography to afford the title compound. LCMS R_{T}=0.673 min, m/z=513.1 [M+H]⁺.

### Step 5

The racemate from Step 4 was separated by SFC to give first eluting fraction (95-i, Rt=2.632 min) and second eluting fraction (95-ii, Rt=2.849 min). LCMS R_{T}=0.687 min, m/z=513.1 [M+H]⁺. (*Absolute stereochemistry not determined.) Step 6:

The solution of 95-i (60 mg, 0.12 mmol) in tetrahydrofuran (3 mL) was added 1-(2,2,2-trifluoroethyl)pyrimidine-2,4-dione (45 mg, 0.23 mmol), diisopropyl azodocarboxylate (47 mg, 0.23 mmol) and triphenylphosphine (61 mg, 0.23 mmol) at 0° C. After stirring at 50° C. for 1 h, the reaction was concentrated under reduced pressure. The residue was dissolved in hydrochloric acid (4 M in dioxane, 3 mL) and stirred at 25° C. for 10 min. The reaction was concentrated under reduced pressure and the residue purified by RP-HPLC to give Example 97a. ¹H NMR (400 MHz, CD₃OD) δ=8.68 (d, J=4.8 Hz, 1H), 7.64 (d, J=8.0 Hz, 1H), 7.54 (s, 1H), 7.44 (d, J=4.8 Hz, 1H), 7.10 (s, 2H), 5.88 (d, J=8.0 Hz, 1H), 5.38 (s, 2H), 4.64 (q, J=8.8 Hz, 2H), 3.52 (q, J=8.0 Hz, 1H), 3.32-3.14 (m, 2H), 2.84 (t, J=6.0 Hz, 2H), 2.48 (s, 1H), 2.04-1.28 (m, 4H), 0.98 (d, J=5.6 Hz, 4H). LCMS R_{T}=0.572 min, m/z=589.1 [M+H]⁺. (*Absolute stereochemistry not determined.)

Example 95b was prepared from 95-ii and 1-(2,2,2-trifluoroethyl)pyrimidine-2,4-dione, following the procedure described in the synthesis of Example 95a. The final product was purified by RP-HPLC. ¹H NMR (400 MHz, CD₃OD) δ=8.56 (d, J=4.8 Hz, 1H), 7.54 (d, J=8.0 Hz, 1H), 7.42 (s, 1H), 7.32 (d, J=4.8 Hz, 1H), 6.98 (d, J=1.2 Hz, 2H), 5.76 (d, J=8.0 Hz, 1H), 5.32-5.22 (m, 2H), 4.58-4.46 (m, 2H), 3.40 (q, J=8.0 Hz, 1H), 3.16-2.98 (m, 2H), 2.72 (t, J=6.0 Hz, 2H), 2.46-2.28 (m, 1H), 1.92-1.14 (m, 4H), 0.88 (s, 4H). LCMS R_{T}=0.576 min, m/z=589.1 [M+H]⁺. (*Absolute stereochemistry not determined.)

### Example 96a and 96b: 1-((7-(6-chloro-1-((3R,4R)-4-fluoropyrrolidin-3-yl)-1,2,3,4-tetrahydroquinolin-8-yl)thieno[3,2-b]pyridin-2-yl)methyl)pyrrolidine-2,5-dione and 1-((7-(6-chloro-1-((3S,4S)-4-fluoropyrrolidin-3-yl)-1,2,3,4-tetrahydroquinolin-8-yl)thieno[3,2-b]pyridin-2-yl)methyl)pyrrolidine-2,5-dione

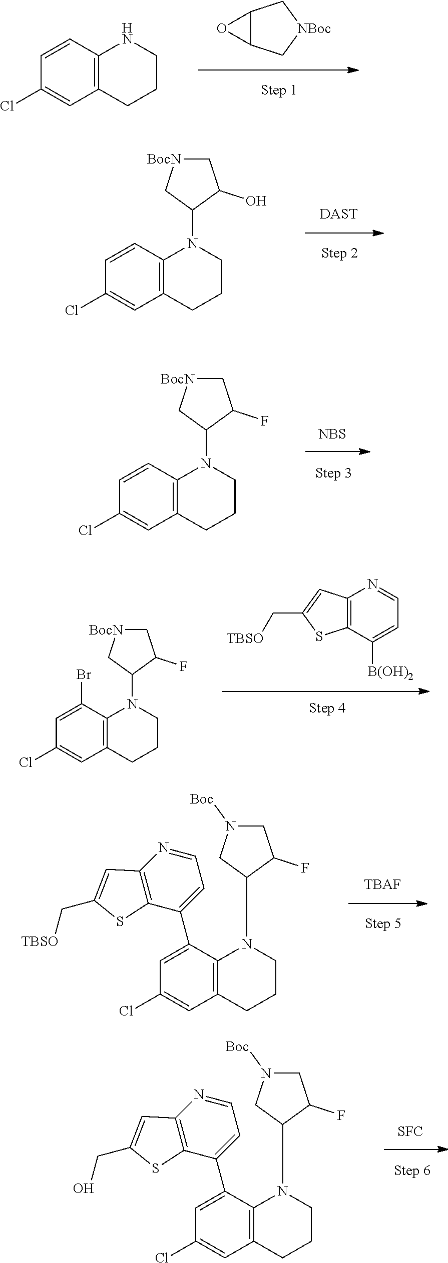

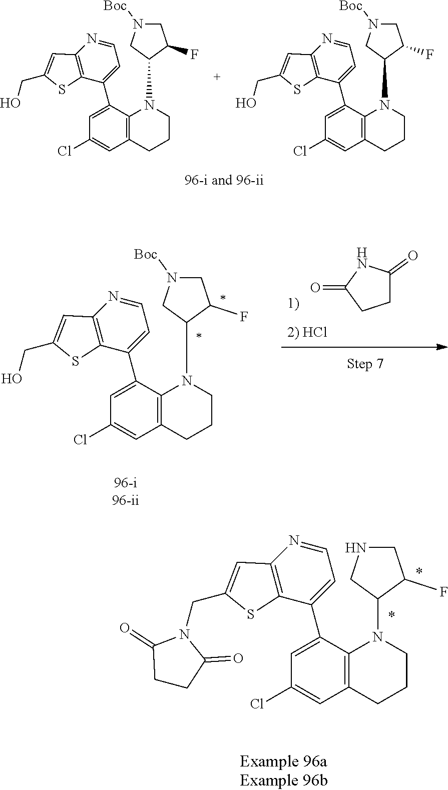 [SMILES: CC(C)(C)OC(=O)N1C[C@@H](F)[C@H](N2CCCc3cc(Cl)cc(-c4ccnc5cc(CO)sc45)c32)C1 ; CC(C)(C)OC(=O)N1C[C@H](F)[C@@H](N2CCCc3cc(Cl)cc(-c4ccnc5cc(CO)sc45)c32)C1 ; CC(C)(C)OC(=O)N1CC(F)C(N2CCCc3cc(Cl)cc(-c4ccnc5cc(CO)sc45)c32)C1 ; O=C1CCC(=O)N1 ; O=C1CCC(=O)N1Cc1cc2nccc(-c3cc(Cl)cc4c3N(C3CNCC3F)CCC4)c2s1]

### Step 1: trans-tert-butyl 3-(6-chloro-3,4-dihydroquinolin-1(2H)-yl)-4-hydroxypyrrolidine-1-carboxylate

To a solution of 6-chloro-1,2,3,4-tetrahydroquinoline (5.0 g, 29.83 mmol) in acetonitrile (5 mL) were added zirconium(IV) tetrachloride (1.4 g, 5.97 mmol) and tert-butyl 6-oxa-3-azabicyclo[3.1.0]hexane-3-carboxylate (6.6 g, 35.8 mmol) at 25° C. After stirring at 25° C. for 2 h, the reaction was filtered and concentrated under reduced pressure. The residue was purified by column chromatography to afford the title compound. LCMS R_{T}=1.025 min, m/z=353.2 [M+H]⁺.

### Step 2: trans-tert-butyl 3-(6-chloro-3,4-dihydroquinolin-1(2H)-yl)-4-fluoropyrrolidine-1-carboxylate

To a solution of trans-tert-butyl 3-(6-chloro-3,4-dihydroquinolin-1(2H)-yl)-4-hydroxypyrrolidine-1-carboxylate (5.0 g, 14.17 mmol) in dichloromethane (50 mL) was added diethyl amino sulfur trifluoride (4.6 g, 28.34 mmol) at 0° C. After stirring at 25° C. for 2 h, the reaction was poured into ice-water and extracted with dichloromethane (30 mL×2). The combined organic layers were washed with brine (50 mL), dried over anhydrous sodium sulfate, filtered and concentrated under reduced pressure. The residue was purified by column chromatography to afford the title compound. LCMS R_{T}=1.096 min, m/z=355.2 [M+H]⁺.

### Step 3: trans-tert-butyl 3-(8-bromo-6-chloro-3,4-dihydroquinolin-1(2H)-yl)-4-fluoropyrrolidine-1-carboxylate

To a solution of trans-tert-butyl 3-(6-chloro-3,4-dihydroquinolin-1(2H)-yl)-4-fluoropyrrolidine-1-carboxylate (1.3 g, 3.66 mmol) in N,N-dimethylformamide (10 mL) was added N-bromosuccinimide (586 mg, 3.3 mmol,) at 25° C. After stirring at 25° C. for 1 h, the reaction was quenched by addition of saturated aqueous ammonium chloride (10 mL), diluted with water (30 mL) and extracted with dichloromethane 45 mL (15 mL×3). The combined organic layers were washed with brine (20 mL), dried over anhydrous sodium sulfate, filtered and concentrated under reduced pressure. The residue was purified by column chromatography to afford the title compound. ¹H NMR (400 MHz, CDCl₃) δ=7.42-7.36 (m, 1H), 7.03-6.94 (m, 1H), 5.42-5.16 (m, 1H), 4.49-4.33 (m, 1H), 3.96-3.74 (m, 2H), 3.59-3.32 (m, 2H), 3.25-3.17 (m, 1H), 3.08-2.98 (m, 1H), 2.85-2.69 (m, 2H), 1.95-1.87 (m, 2H), 1.52-1.45 (m, 9H). LCMS R_{T}=1.155 min, m/z=435.1 [M+H]⁺.

### Step 4: trans-tert-butyl 3-(8-(2-(((tert-butyldimethylsilyl)oxy)methyl)thieno[3,2-b]pyridin-7-yl)-6-chloro-3,4-dihydroquinolin-1(2H)-yl)-4-fluoropyrrolidine-1-carboxylate

To a solution of trans-tert-butyl 3-(8-bromo-6-chloro-3,4-dihydroquinolin-1(2H)-yl)-4-fluoropyrrolidine-1-carboxylate (500 mg, 1.15 mmol) in dioxane (10 mL) and water (2 mL) were added (2-(((tert-butyldimethylsilyl)oxy)methyl)thieno[3,2-b]pyridin-7-yl)boronic acid (559 mg, 1.73 mmol), cesium carbonate (1.1 g, 3.46 mmol) and dichloro[1,1′-bis(diphenylphosphino)ferrocene]palladium(II) (168 mg, 0.23 mmol). After stirring at 120° C. for 2 h under nitrogen atmosphere, the reaction was quenched by addition of saturated aqueous ammonium chloride (15 mL), diluted with water (15 mL) and extracted with ethyl acetate (15 mL×3). The combined organic layers were washed with brine (15 mL), dried over anhydrous sodium sulfate, filtered and concentrated under reduced pressure. The residue was purified by column chromatography to afford the title compound. LCMS R_{T}=1.135 min, m/z=632.2 [M+H]⁺.

### Step 5: (f)-trans-tert-butyl 3-(6-chloro-8-(2-(hydroxymethyl)thieno[3,2-b]pyridin-7-yl)-3,4-dihydroquinolin-1(2H)-yl)-4-fluoropyrrolidine-1-carboxylate

To a solution of trans-tert-butyl 3-(8-(2-(((tert-butyldimethylsilyl)oxy)methyl)thieno[3,2-b]pyridin-7-yl)-6-chloro-3,4-dihydroquinolin-1(2H)-yl)-4-fluoropyrrolidine-1-carboxylate (560 mg, 0.89 mmol) in tetrahydrofuran (10 mL) was added tetrabutylammonium fluoride (1 M in tetrahydrofuran, 0.89 mL). After stirring at 25° C. for 0.5 h, the reaction was concentrated under reduced pressure. The residue was purified by column chromatography to afford the title compound. LCMS R_{T}=0.874 min, m/z=518.1 [M+H]⁺.

### Step 6

The racemate form Step 5 was separated by SFC to give first eluting fraction (96-i, Rt=3.221 min) and second eluting fraction (96-ii, Rt=3.424 min). LCMS R_{T}=0.887 min, m/z=519.8 [M+H]⁺.

### Step 7

To a solution of 96-i (50 mg, 0.1 mmol), succinimide (19 mg, 0.19 mmol) and triphenylphosphine (50 mg, 0.19 mmol) in tetrahydrofuran (2 mL) was added diisopropyl azodicarboxylate (39 mg, 0.19 mmol) at 0° C. After stirring at 25° C. for 1 h, the reaction was concentrated under reduced pressure. The residue was dissolved in hydrochloric acid (4 M in dioxane, 1 mL). After stirring at 25° C. for 30 min, the reaction was concentrated under reduced pressure. The residue was purified by RP-HPLC to afford Example 96a. ¹H NMR (400 MHz, CD₃OD) δ=8.68 (d, J=4.4 Hz, 1H), 7.48-7.44 (m, 2H), 7.19-7.14 (m, 2H), 4.94 (s, 2H), 3.73-3.55 (m, 1H), 3.31-3.21 (m, 4H), 3.13 (s, 2H), 3.04-2.80 (m, 2H), 2.75 (s, 4H), 2.57 (d, J=6.4 Hz, 1H), 2.12-1.89 (m, 2H). LCMS R_{T}=1.622 min, m/z=499.1 [M+H]⁺. (*Absolute stereochemistry not determined.)

Example 96b was prepared from 96-ii, following the procedure described in the synthesis of Example 98a. The final product was purified by RP-HPLC. ¹H NMR (400 MHz, CD₃OD) δ=8.69 (d, J=4.0 Hz, 1H), 7.50-7.42 (m, 2H), 7.21-7.13 (m, 2H), 4.65 (s, 2H), 3.75-3.60 (m, 1H), 3.31-3.11 (m, 4H), 3.10-2.98 (m, 2H), 2.86 (s, 2H), 2.76 (s, 4H), 2.59 (s, 1H), 2.12-1.91 (m, 2H). LCMS R_{T}=0.774 min, m/z=499.1 [M+H]⁺.

### Example 97a and 97b: 1-((7-(6-chloro-1-((3S,4S)-4-methoxypyrrolidin-3-yl)-1,2,3,4-tetrahydroquinolin-8-yl)thieno[3,2-b]pyridin-2-yl)methyl)pyrrolidine-2,5-dione, formic acid salt and 1-((7-(6-chloro-1-((3R,4R)-4-methoxypyrrolidin-3-yl)-1,2,3,4-tetrahydroquinolin-8-yl)thieno[3,2-b]pyridin-2-yl)methyl)pyrrolidine-2,5-dione, formic acid salt

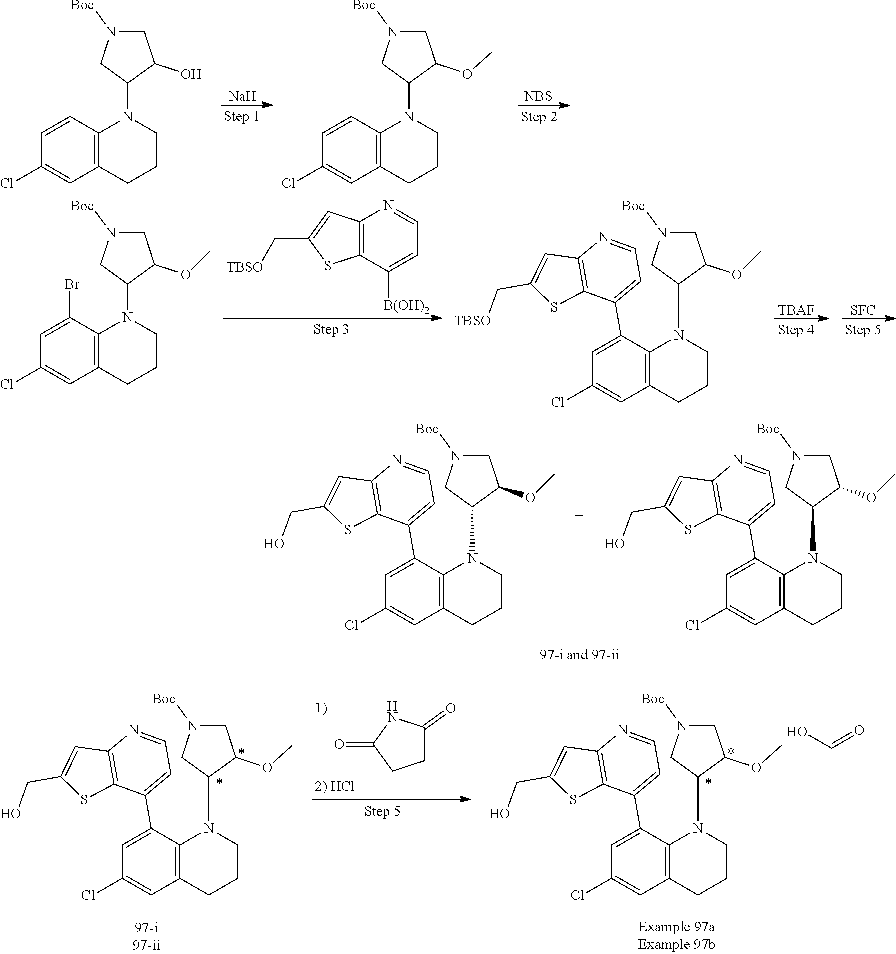

### Step 1: trans-tert-butyl 3-(6-chloro-3,4-dihydroquinolin-1(2H)-yl)-4-methoxypyrrolidine-1-carboxylate

To a solution of trans-tert-butyl 3-(6-chloro-3,4-dihydroquinolin-1(2H)-yl)-4-hydroxypyrrolidine-1-carboxylate (1.8 g, 5.1 mmol) in tetrahydrofuran (30 mL) were added sodium hydride (60% in mineral oil, 244 mg, 6.12 mmol) and iodomethane (2.6 g, 18.36 mmol) at 0° C. After stirring at 0° C. for 2 h, the reaction was quenched by addition of water (2 mL) and filtered. The filtrate was concentrated under reduced pressure and the residue purified by column chromatography to afford the title compound. LCMS R_{T}=2.265 min, m/z=367.3 [M+H]⁺.

### Step 2: trans-tert-butyl 3-(8-bromo-6-chloro-3,4-dihydroquinolin-1(2H)-yl)-4-methoxypyrrolidine-1-carboxylate

To a solution of trans-tert-butyl 3-(6-chloro-3,4-dihydroquinolin-1(2H)-yl)-4-methoxypyrrolidine-1-carboxylate (1.6 g, 4.36 mmol) in N,N-dimethylformamide (30 mL) was added N-bromosuccinimide (776 mg, 4.36 mmol). After stirring at 25° C. for 1 h, the reaction was concentrated under reduced pressure. The residue was purified by column chromatography to afford the title compound. ¹H NMR (400 MHz, CDCl₃) δ=7.35 (s, 1H), 6.95 (s, 1H), 4.22-4.17 (m, 1H), 4.05-3.96 (m, 1H), 3.89-3.75 (m, 2H), 3.30-3.21 (m, 5H), 3.10-2.95 (m, 2H), 2.82-2.69 (m, 2H), 1.88-1.82 (m, 2H), 1.47 (s, 9H). LCMS R_{T}=1.366 min, m/z=447.0 [M+H]⁺.

### Step 3: trans-tert-butyl 3-(8-(2-(((tert-butyldimethylsilyl)oxy)methyl)thieno[3,2-b]pyridin-7-yl)-6-chloro-3,4-dihydroquinolin-1(2H)-yl)-4-methoxypyrrolidine-1-carboxylate

A mixture of trans-tert-butyl 3-(8-bromo-6-chloro-3,4-dihydroquinolin-1(2H)-yl)-4-methoxypyrrolidine-1-carboxylate (700 mg, 1.57 mmol), (2-(((tert-butyldimethylsilyl)oxy)methyl)thieno[3,2-b]pyridin-7-yl)boronic acid (660 mg, 2.04 mmol), cesium carbonate (1.5 g, 4.71 mmol) and dichloro[1,1′-bis(diphenylphosphino)ferrocene]palladium(II) (229 mg, 0.31 mmol) in dioxane (20 mL) and water (2 mL) was stirred at 100° C. for 4 h under nitrogen atmosphere. The reaction was filtered and concentrated under reduced pressure. The residue was purified by column chromatography to afford the title compound. LCMS R_{T}=2.540 min, m/z=644.4 [M+H]⁺.

### Step 4: trans-tert-butyl 3-(6-chloro-8-(2-(hydroxymethyl)thieno[3,2-b]pyridin-7-yl)-3,4-dihydroquinolin-1(2H)-yl)-4-methoxypyrrolidine-1-carboxylate

To a solution of trans-tert-butyl 3-(8-(2-(((tert-butyldimethylsilyl)oxy)methyl)thieno[3,2-b]pyridin-7-yl)-6-chloro-3,4-dihydroquinolin-1(2H)-yl)-4-methoxypyrrolidine-1-carboxylate (1.0 g, 1.55 mmol) in tetrahydrofuran (30 mL) was added tetrabutylammonium fluoride (1 M in tetrahydrofuran, 2.33 mL). After stirring at 25° C. for 1 h, the reaction was concentrated under reduced pressure. The residue was purified by RP-HPLC.

The racemate from Step 4 was separated by SFC to afford first eluting fraction (97-i, Rt=2.010 min) and second eluting fraction (97-ii, Rₜ=2.919 min). LCMS R_{T}=1.861 min, m/z=530.3 [M+H]⁺.

### Step 5

To a solution of 97-i (150 mg, 0.28 mmol) and succinimide (84 mg, 0.85 mmol) in tetrahydrofuran (10 mL) were added triphenylphosphine (222 mg, 0.85 mmol) and diisopropyl azodicarboxylate (172 mg, 0.85 mmol) at 0° C. After stirring at 0° C. for 2 h, the reaction was concentrated under reduced pressure. The residue was dissolved in dioxane (5 mL) and hydrochloric acid (4 M in dioxane, 5 mL) was added. After stirring at 25° C. for 30 min, the reaction was concentrated under reduced pressure. The residue was purified by RP-HPLC to afford Example 97a. ¹H NMR (400 MHz, CD₃OD) δ=8.73 (s, 1H), 8.48 (s, 1H), 7.49 (s, 2H), 7.20 (d, J=3.6 Hz, 2H), 4.97 (s, 2H), 3.62-3.51 (m, 1H), 3.33-3.32 (m, 5H), 3.20-3.15 (m, 2H), 3.20-3.15 (m, 2H), 2.88 (s, 3H), 2.78 (s, 4H), 2.17-1.93 (m, 2H). LCMS R_{T}=1.247 min, m/z=511.2 [M+H]⁺. (*Absolute stereochemistry not determined.)

To a solution of 97-ii (170 mg, 0.32 mmol) and succinimide (95 mg, 0.96 mmol) in tetrahydrofuran (10 mL) were added triphenylphosphine (252 mg, 0.96 mmol) and diisopropyl azodicarboxylate (194 mg, 0.96 mmol) at 0° C. After stirring at 0° C. for 2 h, the reaction was concentrated under reduced pressure. The residue was dissolved in dioxane (5 mL) and hydrochloric acid (4 M in dioxane, 5 mL) was added. After stirring at 25° C. for 30 min, the reaction was concentrated under reduced pressure. The residue was purified by RP-HPLC (10-48% acetonitrile in water and 0.05% hydrochloric acid) to afford Example 97b. ¹H NMR (400 MHz, CD₃OD) δ=8.99 (d, J=5.2 Hz, 1H), 8.07-8.04 (m, 1H), 7.78-7.77 (m, 1H), 7.34-7.33 (m, 2H), 5.09 (s, 2H), 3.49 (s, 1H), 3.33-3.31 (m, 5H), 3.14 (s, 2H), 3.09-3.03 (m, 2H), 2.96-2.90 (m, 3H), 2.80 (s, 4H), 2.05 (s, 2H). LCMS R_{T}=1.265 min, m/z=511.3 [M+H]⁺. (*Absolute stereochemistry not determined.)

### Example 98: (R)-7-(6-chloro-1-(pyrrolidin-3-yl)-1,2,3,4-tetrahydroquinolin-8-yl)-2-((1-methyl-1H-1,2,3-triazol-4-yl)methyl)thieno[3,2-b]pyridine, formic acid salt

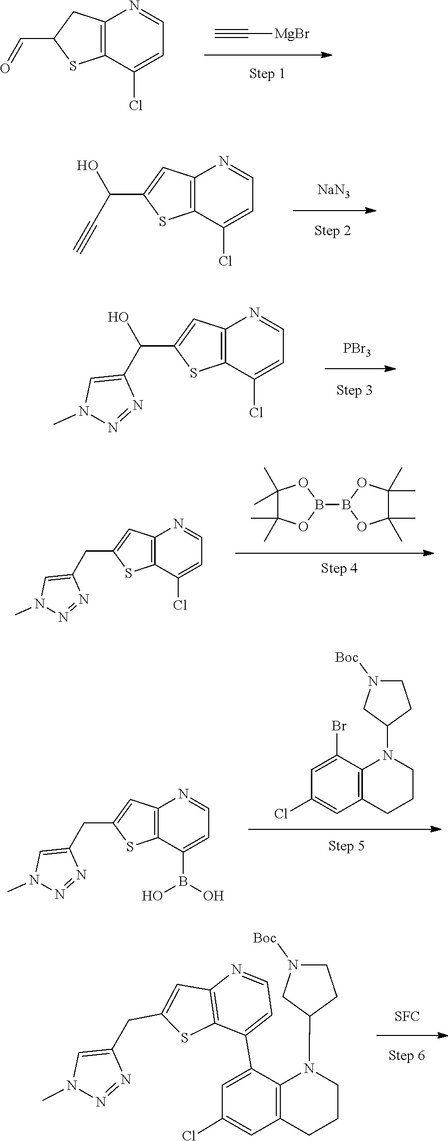

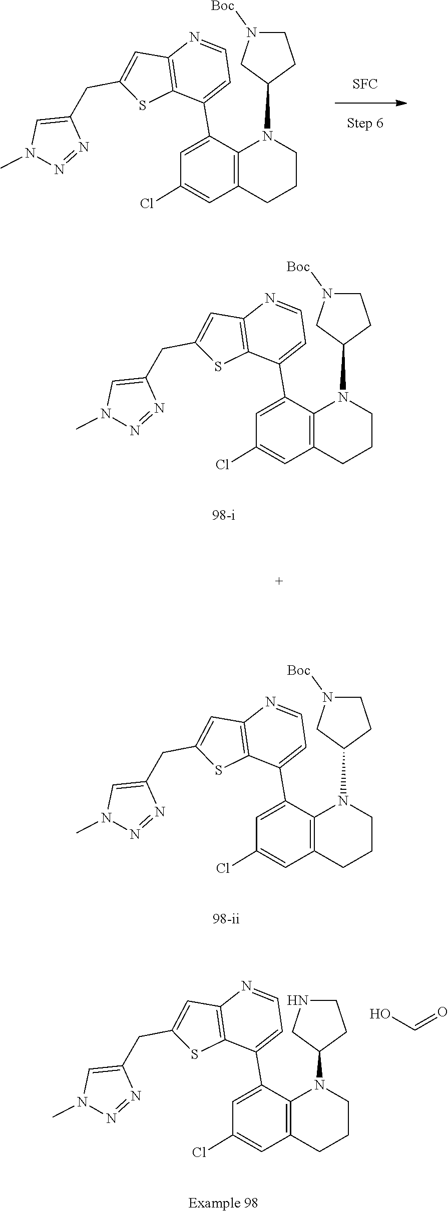

### Step 1: 1-(7-chlorothieno[3,2-b]pyridin-2-yl)prop-2-yn-1-ol

To a solution of 7-chlorothieno[3,2-b]pyridine-2-carbaldehyde (4.7 g, 23.78 mmol) in tetrahydrofuran (30 mL) was added dropwise a solution of ethynylmagnesium bromide (0.5 M in tetrahydrofuran, 71.34 mL) at 0° C. under nitrogen atmosphere. After stirring at 25° C. for 2 h under nitrogen atmosphere, the reaction was quenched by addition of saturated aqueous ammonium chloride (20 mL). Then the mixture was diluted with water (30 mL) and extracted with ethyl acetate (50 mL×2). The combined organic layers were washed with brine (30 mL), dried over anhydrous sodium sulfate, filtered and concentrated under reduced pressure. The residue was purified by column chromatography to afford the title compound. LCMS R_{T}=1.180, m/z=224.0 [M+H]⁺.

### Step 2: (7-chlorothieno[3,2-b]pyridin-2-yl)(1-methyl-1H-1,2,3-triazol-4-yl)methanol

To a solution of sodium ascorbate (443 mg, 2.24 mmol) and 1,10-phenanthroline (20 mg, 0.11 mmol) in ethanol (12 mL) and water (6 mL) was added copper acetate monohydrate (22 mg, 0.11 mmol). After stirring at 25° C. for 5 min under nitrogen atmosphere, 1-(7-chlorothieno[3,2-b]pyridin-2-yl)prop-2-yn-1-ol (500 mg, 2.24 mmol), sodium azide (290 mg, 4.47 mmol) and iodomethane (634 mg, 4.47 mmol) were added to the reaction mixture. After stirring at 25° C. for 16 h under nitrogen atmosphere, the reaction was filtered and concentrated under reduced pressure. The residue was purified by column chromatography to afford the title compound. LCMS R_{T}=0.761, m/z=281.0 [M+H]⁺.

### Step 3: 7-chloro-2-((1-methyl-1H-1,2,3-triazol-4-yl)methyl)thieno[3,2-b]pyridine

To a solution of (7-chlorothieno[3,2-b]pyridin-2-yl)(1-methyl-1H-1,2,3-triazol-4-yl)methanol (500 mg, 1.78 mmol) in tetrahydrofuran (10 mL) was added tribromophosphine (1.9 g, 7.12 mmol) at 25° C. under nitrogen atmosphere. After stirring at 70° C. for 16 h under nitrogen atmosphere, the reaction was quenched by addition of aqueous sodium hydroxide (4 M, 8 mL) and the pH was adjusted to approximately 9-10. The mixture was concentrated under reduced pressure. The residue was diluted with water (20 mL) and extracted with ethyl acetate (30 mL×2). The combined organic layers were washed with brine (20 mL), dried over anhydrous sodium sulfate, filtered and concentrated under reduced pressure. The residue was purified by RP-HPLC to afford the title compound. LCMS R_{T}=1.063, m/z=265.1 [M+H]⁺.

### Step 4: (2-((1-methyl-1H-1,2,3-triazol-4-yl)methyl)thieno[3,2-b]pyridin-7-yl)boronic acid

To a solution of 7-chloro-2-((1-methyl-1H-1,2,3-triazol-4-yl)methyl)thieno[3,2-b]pyridine (100 mg, 0.38 mmol), bis(pinacolato)diboron (192 mg, 0.76 mmol) and potassium acetate (111 mg, 1.13 mmol) in dioxane (1 mL) was added dichloro[1,1′-bis(diphenylphosphino)ferrocene]palladium(II) (113 mg, 0.15 mmol). After stirring at 140° C. for 1 h under nitrogen atmosphere by microwave, the reaction was filtered and concentrated under reduced pressure to afford the crude title compound. It was used for next step without further purification. LCMS R_{T}=1.141, m/z=275.1 [M+H]⁺.

### Step 5: tert-butyl 3-(6-chloro-8-(2-((1-methyl-1H-1,2,3-triazol-4-yl)methyl)thieno[3,2-b]pyridin-7-yl)-3,4-dihydroquinolin-1(2H)-yl)pyrrolidine-1-carboxylate

To a mixture of tert-butyl 3-(8-bromo-6-chloro-3,4-dihydroquinolin-1(2H)-yl)pyrrolidine-1-carboxylate (126 mg, 0.3 mmol), (2-((1-methyl-1H-1,2,3-triazol-4-yl)methyl)thieno[3,2-b]pyridin-7-yl)boronic acid (100 mg, 0.36 mmol) and cesium carbonate (198 mg, 0.61 mmol) in dioxane (4 mL) and water (1 mL) was added dichloro[1,1′-bis(diphenylphosphino)ferrocene]palladium(II) (66 mg, 0.09 mmol). After stirring at 100° C. for 2 h under nitrogen atmosphere, the reaction was filtered, and the filtrate was concentrated under reduced pressure. The residue was purified by RP-TLC (100% ethyl acetate) to afford the title compound. LCMS R_{T}=1.225, m/z=565.4 [M+H]⁺.

### Step 6

The racemate from Step 5 was separated by SFC to afford first eluting fraction (98-i, Rt=2.919 min) and second eluting fraction (98-ii, Rₜ=3.424 min). LCMS R_{T}=1.232 min, m/z=565.4 [M+H]⁺.

### Step 7

Compound 98-i (22 mg, 0.04 mmol) was dissolved in dioxane (5 mL) and hydrochloric acid (4 M in dioxane, 1.8 mL) was added. After stirring at 25° C. for 30 min, the reaction was concentrated under reduced pressure. The residue was purified by RP-HPLC to afford Example 98. ¹H NMR (400 MHz, CD₃OD) δ=8.67 (d, J=4.8 Hz, 1H), 8.50 (s, 1H), 7.87 (s, 1H), 7.42 (d, J=4.8 Hz, 1H), 7.35 (s, 1H), 7.17-7.11 (m, 2H), 4.40 (s, 2H), 4.09 (s, 3H), 3.67 (q, J=8.4 Hz, 1H), 3.26-3.06 (m, 3H), 2.92-2.20 (m, 5H), 1.98-1.47 (m, 4H). LCMS R_{T}=0.946 min, m/z=465.3 [M+H]⁺. The absolute configuration was tentatively assigned based on the relative potency of the pair enantiomers in biology assays.

### Example 99: (S)-7-(6-chloro-1-(pyrrolidin-3-yl)-1,2,3,4-tetrahydroquinolin-8-yl)-2-((1-methyl-1H-1,2,3-triazol-4-yl)methyl)thieno[3,2-b]pyridine, formic acid salt

Example 99 was prepared from 98-ii, following the procedure described in the synthesis of Example 98. The final compound was purified by RP-HPLC. ¹H NMR (400 MHz, CD₃OD) δ=8.67 (d, J=4.8 Hz, 1H), 8.49 (s, 1H), 7.87 (s, 1H), 7.43 (d, J=4.8 Hz, 1H), 7.35 (s, 1H), 7.14 (d, J=8.0 Hz, 2H), 4.40 (s, 2H), 4.09 (s, 3H), 3.77-3.59 (m, 1H), 3.26-3.08 (m, 3H), 2.95-2.23 (m, 5H), 1.98-1.51 (m, 4H). LCMS R_{T}=0.951 min, m/z=465.3 [M+H]⁺. The absolute configuration was tentatively assigned based on the relative potency of the pair enantiomers in biology assays.

### Example 100: (R)-3-((7-(6-chloro-1-(pyrrolidin-3-yl)-1,2,3,4-tetrahydroquinolin-8-yl)thieno[3,2-b]pyridin-2-yl)methyl)-1-(2-methoxyethyl)pyrimidine-2,4(1H,3H)-dione, formic acid salt

Example 100 was prepared from 55-ii and 1-(2-methoxyethyl)pyrimidine-2,4-dione following the procedure described in the synthesis of Example 1. The final product was purified by RP-HPLC. ¹H NMR (400 MHz, CD₃OD) δ=8.72 (d, J=4.8 Hz, 1H), 8.53 (s, 1H), 7.59 (d, J=8.0 Hz, 1H), 7.55-7.41 (m, 2H), 7.32-7.10 (m, 2H), 5.76 (d, J=8.0 Hz, 1H), 5.41 (s, 2H), 3.99 (t, J=4.8 Hz, 2H), 3.76-3.56 (m, 3H), 3.32-3.08 (m, 7H), 3.03-2.33 (m, 4H), 2.00-1.20 (m, 4H). LCMS R_{T}=0.710 min, m/z=552.3 [M+H]⁺

### Example 101: (S)-3-((7-(6-chloro-1-(pyrrolidin-3-yl)-1,2,3,4-tetrahydroquinolin-8-yl)thieno[3,2-b]pyridin-2-yl)methyl)-1-(2,2-difluoroethyl)pyrimidine-2,4(1H,3H)-dione, formic acid salt

Example 101 was prepared from 1-(2,2-difluoroethyl)pyrimidine-2,4-dione and 55-i, following the procedure described in the synthesis of Example 1. The final product was purified by RP-HPLC. ¹H NMR (400 MHz, CD₃OD) δ=8.72 (d, J=4.8 Hz, 1H), 8.54 (s, 1H), 7.63 (d, J=8.0 Hz, 1H), 7.57-7.43 (m, 2H), 7.19 (br d, J=4.4 Hz, 2H), 6.44-5.96 (m, 1H), 5.84 (d, J=8.0 Hz, 1H), 5.40 (s, 2H), 4.35-4.15 (m, 2H), 3.76-3.62 (m, 1H), 3.32-2.46 (m, 8H), 2.15-1.20 (m, 4H) LCMS R_{T}=1.014 min, m/z=558.3 [M+H]⁺

### Example 102: (S)-1-((7-(6-chloro-1-(1-isobutylpyrrolidin-3-yl)-1,2,3,4-tetrahydroquinolin-8-yl)thieno[3,2-b]pyridin-2-yl)methyl)pyrrolidine-2,5-dione, formic acid salt

To a solution of (S)-1-((7-(6-chloro-1-(pyrrolidin-3-yl)-1,2,3,4-tetrahydroquinolin-8-yl)thieno[3,2-b]pyridin-2-yl)methyl)pyrrolidine-2,5-dione (100 mg, 0.21 mmol) in 1,2-dichloroethane (2 mL) was added isobutyraldehyde (81 mg, 0.4 mmol), sodium borohydride acetate (88 mg, 0.42 mmol) and acetic acid (2 mg, 0.04 mmol). After stirred at 25° C. for 1 hr, the reaction mixture was concentrated under reduced pressure. The residue was purified by RP-HPLC to afford the title compound. ¹H NMR (400 MHz, CD₃OD) δ=8.72 (d, J=4.8 Hz, 1H), 8.41 (s, 1H), 7.51-7.45 (m, 2H), 7.20-7.14 (m, 2H), 4.95 (s, 2H), 3.76-3.65 (m, 1H), 3.26 (d, J=5.2 Hz, 4H), 2.88 (t, J=6.4 Hz, 5H), 2.76 (s, 6H), 2.01-1.64 (m, 4H), 0.92-0.83 (m, 6H). LCMS R_{T}=0.781 min, m/z=537.2 [M+H]⁺.

### Example 103: (S)-3-((7-(6-chloro-1-(pyrrolidin-3-yl)-1,2,3,4-tetrahydroquinolin-8-yl)thieno[3,2-b]pyridin-2-yl)methyl)-1-(2,2-difluoropropyl)pyrimidine-2,4(1H,3H)-dione, formic acid salt

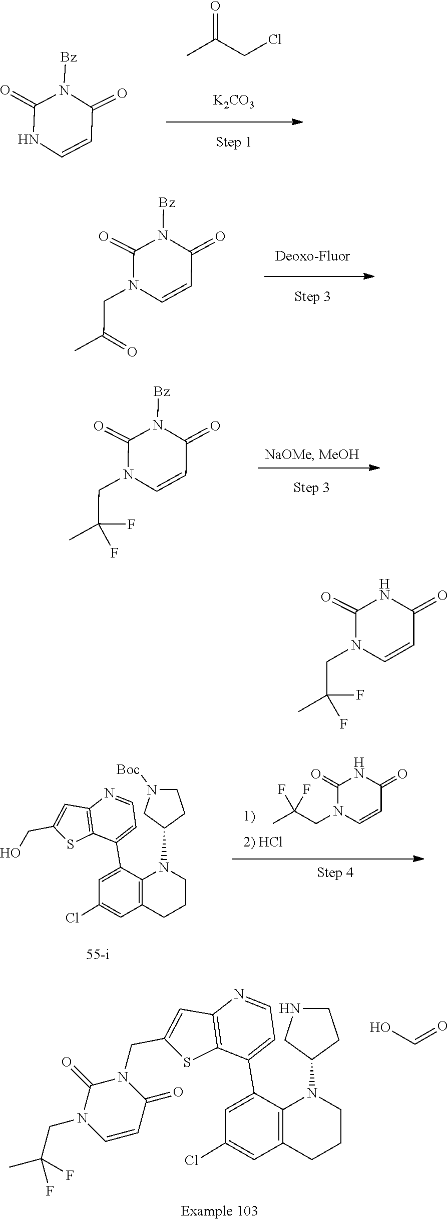

### Step 1: 3-benzoyl-1-(2-oxopropyl)pyrimidine-2,4(1H,3H)-dione

To a solution of 3-benzoylpyrimidine-2,4(1H,3H)-dione (600 mg, 2.78 mmol) in DMF (5 mL) was added 1-chloropropan-2-one (513 mg, 5.55 mmol) and potassium carbonate (767 mg, 5.55 mmol). After stirred at 50° C. for 2 h, the reaction mixture was filtered and the filtrate was concentrated under reduced pressure. The mixture was diluted with water (10 mL) and extracted with ethyl acetate (15 mL×2). The combined organic layers were washed with brine (15 mL), dried over anhydrous sodium sulfate and concentrated under reduced pressure. The residue was purified by column chromatography to afford the title compound. LCMS R_{T}=0.740 min, m/z=273.1 [M+H]⁺.

### Step 2: 3-benzoyl-1-(2,2-difluoropropyl)pyrimidine-2,4(1H,3H)-dione

To a solution of 3-benzoyl-1-(2-oxopropyl)pyrimidine-2,4(1H,3H)-dione (530 mg, 1.95 mmol) in dichloromethane (5 mL) was added bis(2-methoxyethyl)amino sulfur trifluoride (646 mg, 2.92 mmol) at 0° C. After stirred at 25° C. for 16 h, the reaction mixture was quenched by addition of saturated aqueous sodium bicarbonate (10 mL) and extracted with ethyl acetate (10 mL×3). The combined organic layers were washed with brine (20 mL), dried over anhydrous sodium sulfate and concentrated under reduced pressure. The residue was purified by column chromatography (silica gel, 100-200 mesh, 0-10% methanol in dichloromethane) to afford the title compound. LCMS R_{T}=0.867 min, m/z=295.0 [M+H]⁺.

### Step 3: 1-(2,2-difluoropropyl)pyrimidine-2,4(1H,3H)-dione

A mixture of 3-benzoyl-1-(2,2-difluoropropyl)pyrimidine-2,4(1H,3H)-dione (400 mg, 1.36 mmol) and sodium methoxide (147 mg, 2.72 mmol) in methanol (5 mL) was stirred at 25° C. for 3 h. The reaction mixture was purified by RP-HPLC to afford the title compound. LCMS R_{T}=0.297 min, m/z=191.0 [M+H]⁺.

### Step 4

Example 103 was prepared from 1-(2,2-difluoropropyl)pyrimidine-2,4(1H,3H)-dione and 55-i, following the procedure described in the synthesis of Example 1. The final product was purified by RP-HPLC. ¹H NMR (400 MHz, CD₃OD) δ=8.70 (d, J=4.4 Hz, 1H), 8.52 (s, 1H), 7.75-7.38 (m, 3H), 7.16 (d, J=4.8 Hz, 2H), 5.82 (d, J=7.6 Hz, 1H), 5.39 (s, 2H), 4.28 (t, J=14.0 Hz, 2H), 3.66 (s, 1H), 3.30-2.99 (m, 4H), 2.94-2.56 (m, 4H), 1.93 (s, 4H), 1.65 (t, J=18.8 Hz, 4H). LCMS R_{T}=0.793 min, m/z=572.1 [M+H]⁺.

### Example 104: (S)-3-((7-(6-chloro-1-(pyrrolidin-3-yl)-1,2,3,4-tetrahydroquinolin-8-yl)thieno[3,2-b]pyridin-2-yl)methyl)-1-(3,3-difluorobutyl)pyrimidine-2,4(1H,3H)-dione, formic acid salt

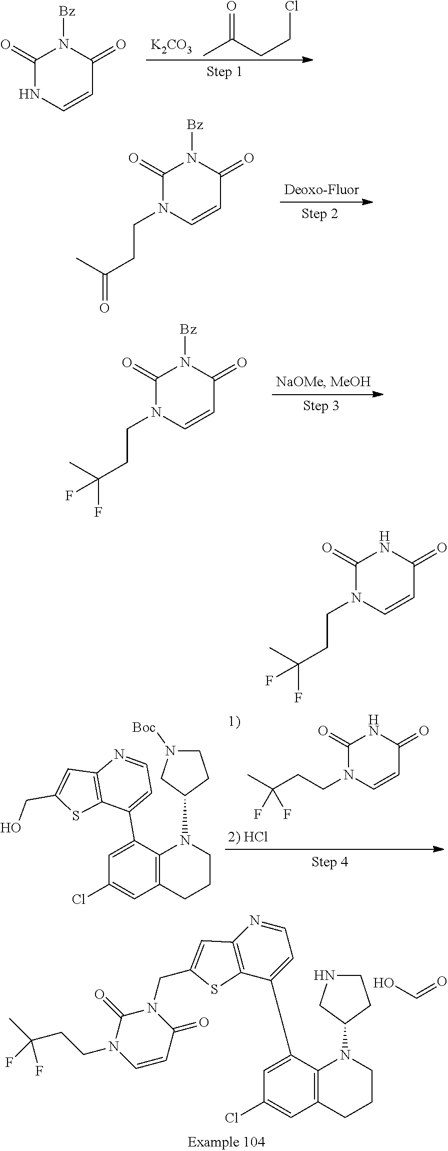

### Step 1: 3-benzoyl-1-(3-oxobutyl)pyrimidine-2,4(1H,3H)-dione

A mixture of 3-benzoylpyrimidine-2,4(1H,3H)-dione (200 mg, 0.93 mmol) and potassium carbonate (281 mg, 2.04 mmol) in DMF (2 mL) was stirred at 25° C. for 0.5 h, followed by the addition of 4-chlorobutan-2-one (197 mg, 1.85 mmol). After stirred at 50° C. for 6 h, the reaction mixture was concentrated under reduced pressure to afford the title compound. LCMS R_{T}=0.760 min, m/z=287.1 [M+H]⁺.

### Step 2: 3-benzoyl-1-(3,3-difluorobutyl)pyrimidine-2,4(1H,3H)-dione

To a solution of 3-benzoyl-1-(3-oxobutyl)pyrimidine-2,4(1H,3H)-dione (250 mg, 0.87 mmol) in dichloromethane (5 mL) was added bis(2-methoxyethyl)amino sulfur trifluoride (966 mg, 4.37 mmol) at 0° C. After stirred for at 25° C. 16 h, the reaction mixture was concentrated under reduced pressure. The residue was purified by RP-TLC (100% ethyl acetate) to afford the title compound. LCMS R_{T}=1.003 min, m/z=308.9 [M+H]⁺.

### Step 3: 1-(3,3-difluorobutyl)pyrimidine-2,4(1H,3H)-dione

To a solution of 3-benzoyl-1-(3,3-difluorobutyl)pyrimidine-2,4-dione (140 mg, 0.45 mmol) in methanol (1 mL) was added sodium methoxide (73 mg, 1.36 mmol) at 20° C. After stirred at 20° C. for 3 h, the reaction mixture was concentrated under reduced pressure. The residue was purified by RP-HPLC to afford the title compound. LCMS R_{T}=1.518 min, m/z=204.8 [M+H]⁺.

### Step 4

Example 104 was prepared from 1-(3,3-difluorobutyl)pyrimidine-2,4(1H,3H)-dione and 55-i, following the procedure described in the synthesis of Example 1. The final product was purified by RP-HPLC to afford the title compound. ¹H NMR (400 MHz, CD₃OD) δ=8.70 (d, J=4.8 Hz, 1H), 8.52 (s, 1H), 7.62 (d, J=8.0 Hz, 1H), 7.51 (s, 1H), 7.47 (d, J=4.8 Hz, 1H), 7.19-7.15 (m, 2H), 5.77 (d, J=8.0 Hz, 1H), 5.38 (s, 2H), 4.01 (t, J=7.2 Hz, 2H), 3.67 (q, J=8.4 Hz, 1H), 3.29-3.04 (m, 4H), 2.88 (t, J=6.4 Hz, 2H), 2.71 (s, 2H), 2.38-2.25 (m, 2H), 1.94 (q, J=6.0 Hz, 3H), 1.63 (t, J=18.8 Hz, 4H). LCMS R_{T}=0.995 min, m/z=586.2 [M+H]⁺.

### Example 105: (S)-2-((7-(6-chloro-1-(pyrrolidin-3-yl)-1,2,3,4-tetrahydroquinolin-8-yl)thieno[3,2-b]pyridin-2-yl)methyl)-5-(2,2-difluoroethyl)pyridazin-3(2H)-one, formic acid salt

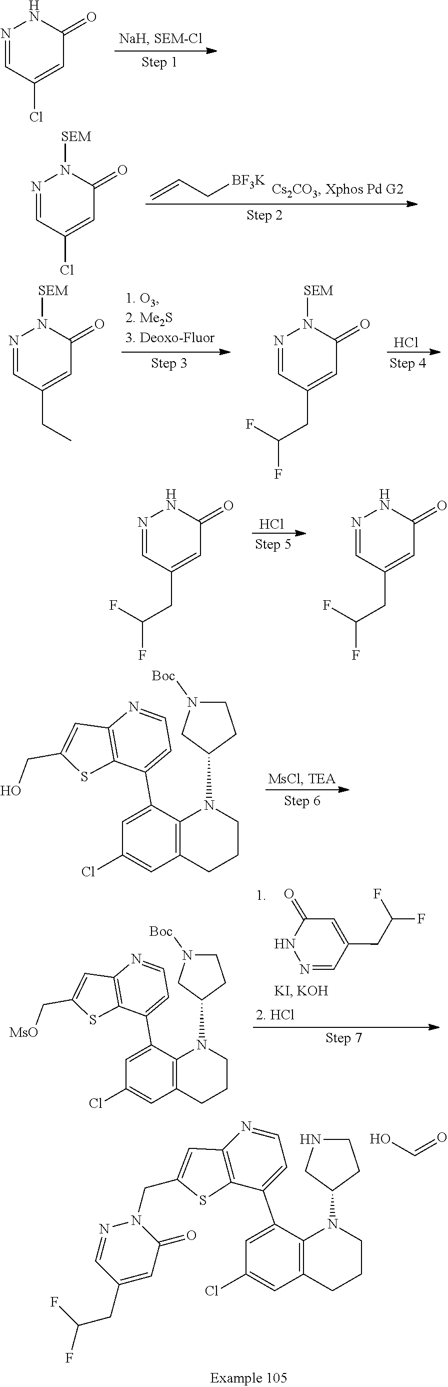

### Step 1: 5-chloro-2-((2-(trimethylsilyl)ethoxy)methyl)pyridazin-3(2H)-one

To a solution of 5-chloropyridazin-3(2H)-one (1.5 g, 11.49 mmol) in DMF (100 mL) was added sodium hydride (551 mg, 13.79 mmol, 60%) at 25° C. After stirred at 25° C. for 1 h under nitrogen atmosphere, (2-(chloromethoxy)ethyl)trimethylsilane (2.87 g, 17.24 mmol) was added to the reaction mixture. After stirred at 25° C. for 4 h under nitrogen atmosphere, the reaction mixture was quenched by addition of water (30 mL) and extracted with ethyl acetate (20 mL×2). The combined organic layers were washed with brine (20 mL), dried over anhydrous sodium sulfate and concentrated under reduced pressure. The residue was purified by column chromatography to afford the title compound. ¹H NMR (400 MHz, CD₃OD) δ=7.98 (d, J=2.4 Hz, 1H), 7.14 (d, J=2.4 Hz, 1H), 5.43 (s, 2H), 3.77-3.67 (m, 2H), 0.98-0.89 (m, 2H), 0.07-0.01 (m, 9H).

### Step 2: 5-allyl-2-((2-(trimethylsilyl)ethoxy)methyl)pyridazin-3(2H)-one

To a solution of 5-chloro-2-((2-(trimethylsilyl)ethoxy)methyl)pyridazin-3(2H)-one (770 mg, 2.95 mmol), potassium allyltrifluoroborate (1.31 g, 8.86 mmol) and cesium carbonate (2.89 g, 8.86 mmol) in dioxane (20 mL) and water (5 mL) was added (2-dicyclohexylphosphino-2′,4′,6′-triisopropyl-1,1′-biphenyl)[2-(2′-amino-1,1′-biphenyl)]palladium(II) (124.96 mg, 0.15 mmol). After stirred at 100° C. for 3 h under nitrogen atmosphere, the reaction mixture was filtered and the filtrate was concentrated under reduced pressure. The residue was diluted with water (15 mL) and extracted with ethyl acetate (25 mL×2). The combined organic layers were washed with brine (15 mL), dried over anhydrous sodium sulfate and concentrated under reduced pressure. The residue was purified by column chromatography to give afford the title compound. ¹H NMR (400 MHz, CDCl₃) δ=7.65 (d, J=2.0 Hz, 1H), 6.72 (d, J=0.8 Hz, 1H), 5.46 (s, 2H), 3.73-3.68 (m, 2H), 3.29-3.20 (m, 2H), 1.00-0.96 (m, 2H), 0.00 (s, 9H).

### Step 3: 5-(2,2-difluoroethyl)-2-((2-(trimethylsilyl)ethoxy)methyl)pyridazin-3(2H)-one

A solution of 5-allyl-2-((2-(trimethylsilyl)ethoxy)methyl)pyridazin-3(2H)-one (300 mg, 1.13 mmol) in methanol (3 mL) was stirred at −78° C. for 10 min under ozone atmosphere for 10 min. Then the reaction mixture was degassed and purged with nitrogen. The reaction mixture was then added dimethylsulfide (280 mg, 4.50 mmol) at −78° C. After stirred at 25° C. for 4 h under nitrogen atmosphere, the reaction mixture was quenched by addition of methanol (10 mL) and the mixture was concentrated under reduced pressure. The residue was dissolved in dichloromethane (3 mL) and bis(2-methoxyethyl)amino sulfur trifluoride (1.24 g, 5.59 mmol) was added. After stirred at 25° C. for 2 h under nitrogen atmosphere, the reaction mixture was quenched by addition of water (10 mL) and extracted with dichloromethane (10 mL×2). The combined organic layers were washed with brine (10 mL), dried over anhydrous sodium sulfate and concentrated under reduced pressure. The residue was purified by RP-TLC to afford the title compound. ¹H NMR (400 MHz, CDCl₃) δ=7.72 (d, J=2.0 Hz, 1H), 6.85 (s, 1H), 6.18-5.85 (m, 1H), 5.47 (s, 2H), 3.74-3.70 (m, 2H), 3.04 (m, 2H), 1.00-0.95 (m, 2H), 0.00 (s, 9H).

### Step 4: 5-(2,2-difluoroethyl)pyridazin-3(2H)-one

To a solution of 5-(2,2-difluoroethyl)-2-((2-(trimethylsilyl)ethoxy)methyl)pyridazin-3(2H)-one (90 mg, 0.31 mmol) in methanol (0.8 mL) was added hydrochloric acid (12 M, 0.77 mL). After stirred at 90° C. for 4 h under nitrogen atmosphere, the reaction mixture was filtered and the filtrate was concentrated under reduced pressure. The residue was purified by RP-HPLC to afford the title compound. ¹H NMR (400 MHz, CDCl₃) δ=11.54 (s, 1H), 7.76 (d, 1.2 Hz, 1H), 6.89 (m, 1H), 6.20-5.87 (m, 1H), 3.06 (m, 2H).

### Step 5: tert-butyl (S)-3-(6-chloro-8-(2-(((methylsulfonyl)oxy)methyl)thieno[3,2-b]pyridin-7-yl)-3,4-dihydroquinolin-1(2H)-yl)pyrrolidine-1-carboxylate

To a solution of tert-butyl (S)-3-(6-chloro-8-(2-(hydroxymethyl)thieno[3,2-b]pyridin-7-yl)-3,4-dihydroquinolin-1(2H)-yl)pyrrolidine-1-carboxylate (50 mg, 0.1 mmol) and triethylamine (30 mg, 0.3 mmol) in dichloromethane (1 mL) was added methanesulfonyl chloride (23 mg, 0.2 mmol) at 0° C. under nitrogen atmosphere. After stirred at 25° C. for 0.5 h under nitrogen atmosphere, the reaction mixture was quenched by addition of saturated aqueous sodium bicarbonate (2 mL) and water (2 mL), then extracted with dichloromethane (4 mL×2). The combined organic layers were washed with brine (4 mL), dried over anhydrous sodium sulfate and concentrated under reduced pressure to afford the title compound. LCMS R_{T}=2.319 min, m/z=578.0 [M+H]⁺.

### Step 6

To a solution of tert-butyl (S)-3-(6-chloro-8-(2-(((methylsulfonyl)oxy)methyl)thieno[3,2-b]pyridin-7-yl)-3,4-dihydroquinolin-1(2H)-yl)pyrrolidine-1-carboxylate (58 mg, 0.1 mmol) and 5-(2,2-difluoroethyl)pyridazin-3(2H)-one (32 mg, 0.2 mmol) in DMF (1 mL) was added potassium iodide (25 mg, 0.15 mmol) and potassium hydroxide (17 mg, 0.31 mmol). After stirred at 25° C. for 1 h under nitrogen atmosphere, the reaction mixture was diluted with water (10 mL) and extracted with dichloromethane (20 mL×2). The combined organic layers were washed with brine (20 mL), dried over anhydrous sodium sulfate and concentrated under reduced pressure. The residue was dissolved in dioxane (5 mL) and added hydrochloric acid in dioxane (4 M, 1.8 mL). After stirred at 25° C. for 30 min, the reaction mixture was concentrated under reduced pressure. The residue was purified by RP-HPLC to afford Example 105. ¹H NMR (400 MHz, CD₃OD) δ=8.72 (d, J=4.8 Hz, 1H), 8.51 (s, 1H), 7.95 (d, J=1.6 Hz, 1H), 7.54 (s, 1H), 7.49 (d, J=4.4 Hz, 1H), 7.17 (d, J=3.6 Hz, 2H), 6.96 (s, 1H), 6.35-6.01 (m, 1H), 5.63 (s, 2H), 3.65 (t, J=8.4 Hz, 1H), 3.26-3.02 (m, 6H), 2.87 (t, J=6.0 Hz, 2H), 2.71 (s, 2H), 1.96-1.54 (m, 4H). LCMS R_{T}=1.660 min, m/z=542.3 [M+H]⁺.

### Example 106: (S)-2-((7-(6-chloro-1-(pyrrolidin-3-yl)-1,2,3,4-tetrahydroquinolin-8-yl)thieno[3,2-b]pyridin-2-yl)methyl)-6,7-dihydropyrrolo[1,2-c]pyrimidine-1,3(2H,5H)-dione, formic acid salt

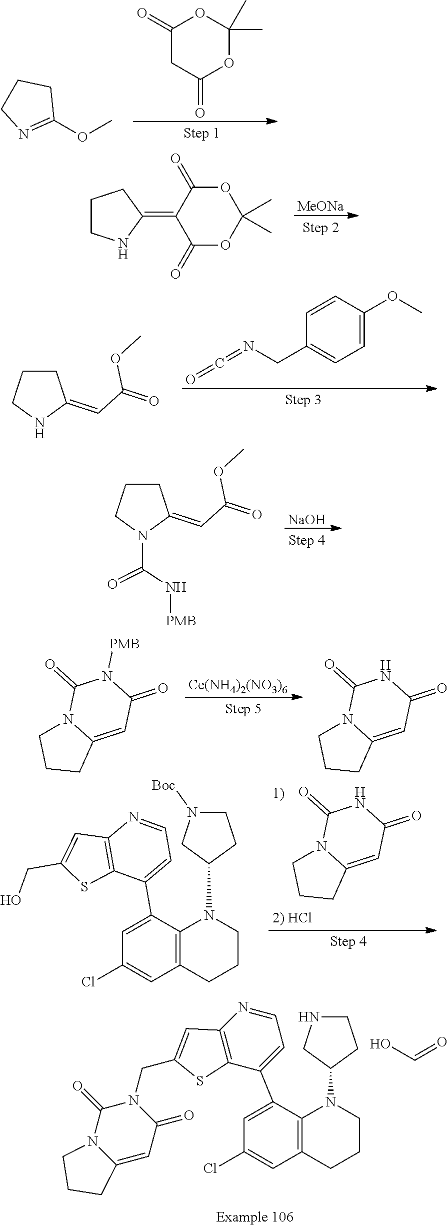

### Step 1: 2,2-dimethyl-5-(pyrrolidin-2-ylidene)-1,3-dioxane-4,6-dione

To a solution of 5-methoxy-3,4-dihydro-2H-pyrrole (10.0 g, 100.88 mmol) and 2,2-dimethyl-1,3-dioxane-4,6-dione (16.0 g, 110.96 mmol) in toluene (50 mL) was added triethylamine (20.4 g, 201.75 mmol). After stirred at 20° C. for 16 h, the reaction mixture was concentrated under reduced pressure. The residue was purified by column chromatography to afford the title compound. ¹H NMR (400 MHz, CDCl₃) δ=10.10 (s, 1H), 3.75-3.66 (m, 2H), 3.39-3.35 (m 2H), 2.19-2.11 (m 2H), 1.66 (d, J=2.8 Hz, 6H).

### Step 2: methyl 2-(pyrrolidin-2-ylidene)acetate

To a solution of 2,2-dimethyl-5-pyrrolidin-2-ylidene-1,3-dioxane-4,6-dione (16.6 g, 78.59 mmol) in methyl alcohol (300 mL) was added sodium methoxide (21.2 g, 392.97 mmol) at 25° C. After stirred at 25° C. for 12 h, the reaction mixture was filtered and the filtrate was concentrated under reduced pressure to give a residue. The residue was purified by column chromatography to afford the title compound. ¹H NMR (400 MHz, CDCl₃) δ=7.88 (s, 1H), 4.52 (s, 1H), 3.60 (s, 3H), 3.49 (t, J=7.6 Hz, 2H), 2.57 (t, J=7.6 Hz, 2H), 1.99-1.93 (m, 2H).

### Step 3: methyl 2-(1-((4-methoxybenzyl)carbamoyl)pyrrolidin-2-ylidene)acetate

To a solution of methyl 2-(pyrrolidin-2-ylidene)acetate (4.0 g, 28.34 mmol) in trichloromethane (10 mL) was added 1-(isocyanatomethyl)-4-methoxy-benzene (5.1 g, 31.17 mmol) at 25° C. After stirred at 60° C. for 12 h, the reaction mixture was filtered and the filtrate was concentrated under reduced pressure. The residue was purified by column chromatography to afford the title compound. LCMS R_{T}=1.535 min, m/z=305.3 [M+H]⁺.

### Step 4: 2-(4-methoxybenzyl)-6,7-dihydropyrrolo[1,2-c]pyrimidine-1,3(2H,5H)-dione

To a solution of methyl 2-(1-((4-methoxybenzyl)carbamoyl)pyrrolidin-2-ylidene)acetate (680 mg, 2.23 mmol) in ethyl alcohol (10 mL) and water (10 mL) was added sodium hydroxide (179 mg, 4.47 mmol). After stirred at 80° C. for 1 h, the reaction mixture was concentrated under reduced pressure. The residue was purified by column chromatography to afford the title compound. LCMS R_{T}=1.114 min, m/z=273.3 [M+H]⁺.

### Step 5: 6,7-dihydropyrrolo[1,2-c]pyrimidine-1,3(2H,5H)-dione

To a solution of 2-[(4-methoxyphenyl)methyl]-6,7-dihydro-5H-pyrrolo[1,2-c]pyrimidine-1,3-dione (150 mg, 0.55 mmol) in acetonitrile (15 mL) and water (5 mL) was added ceric ammonium nitrate (906 mg, 1.65 mmol). After stirred at 25° C. for 12 h, the reaction mixture was filtered and the filtrate was concentrated under reduced pressure to afford the title compound. LCMS R_{T}=0.329 min, m/z=153.3 [M+H]⁺.

### Step 6

To a solution of tert-butyl (3S)-3-[6-chloro-8-[2-(hydroxymethyl)thieno[3,2-b]pyridin-7-yl]-3,4-dihydro-2H-quinolin-1-yl]pyrrolidine-1-carboxylate (90 mg, 0.18 mmol) and 6,7-dihydro-5H-pyrrolo[1,2-c]pyrimidine-1,3-dione (30 mg, 0.18 mmol) in THF (8 mL) was added diisopropylazodicarboxylate (109 mg, 0.54 mmol) and triphenylphosphine (142 mg, 0.54 mmol). After stirred at 50° C. for 3 h, the reaction mixture was concentrated under reduced pressure to remove the solvent. The residue was dissolved in dioxane (5 mL) and then was added hydrochloric acid (5 mL, 4 M in dioxane). After stirred at 25° C. for 30 min, the reaction mixture was concentrated under reduced pressure. The residue was purified by preparative HPLC to afford Example 105. ¹H NMR (400 MHz, CD₃OD) δ=8.69-8.66 (m, 1H), 8.48 (s, 1H), 7.50 (s, 1H), 7.45 (d, J=4.4 Hz, 1H), 7.16-7.12 (m 2H), 5.70 (s, 1H), 5.33 (s, 2H), 3.94 (t, J=7.2 Hz, 2H), 3.67 (s, 1H), 3.21-3.20 (m, 5H), 3.00 (t, J=7.2 Hz, 2H), 2.87 (t, J=6.4 Hz, 2H), 2.72-2.18 (m, 3H), 2.16-2.12 (m, 2H), 1.92 (t, J=5.6 Hz, 2H). LCMS R_{T}=1.312 min, m/z=534.3 [M+H]⁺.

### Example 107a and 107b: 8-(2-((2,6-dioxo-3-(2,2,2-trifluoroethyl)-2,3-dihydropyrimidin-1(6H)-yl)methyl)thieno[3,2-b]pyridin-7-yl)-1-((S)-1-((1R,2R)-2-hydroxycyclobutyl)pyrrolidin-3-yl)-1,2,3,4-tetrahydroquinoline-6-carbonitrile, formic acid salt 8-(2-((2,6-dioxo-3-(2,2,2-trifluoroethyl)-2,3-dihydropyrimidin-1(6H)-yl)methyl)thieno[3,2-b]pyridin-7-yl)-1-((S)-1-((1S,2S)-2-hydroxycyclobutyl)pyrrolidin-3-yl)-1,2,3,4-tetrahydroquinoline-6-carbonitrile, formic acid salt

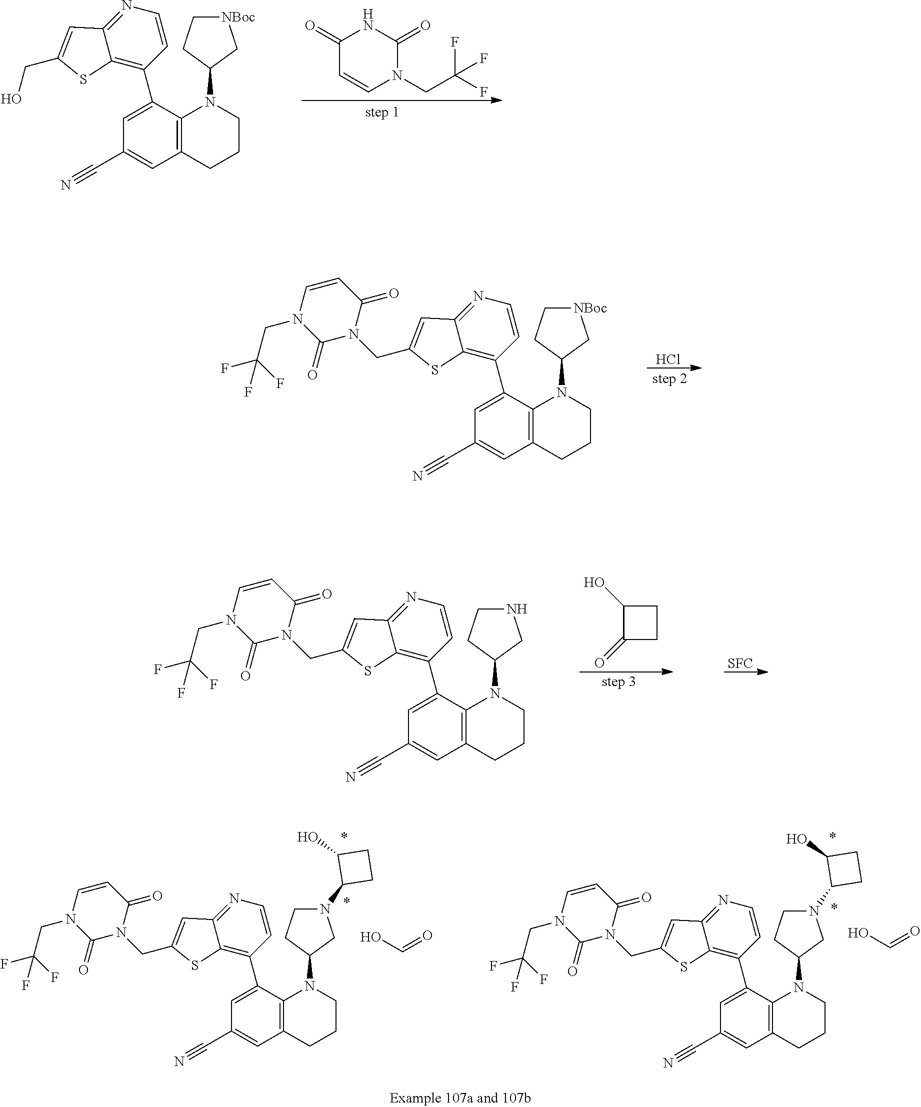

### Step 1: (S)-tert-butyl 3-(6-cyano-8-(2-((2,6-dioxo-3-(2,2,2-trifluoroethyl)-2,3-dihydropyrimidin-1(6H)-yl)methyl)thieno[3,2-b]pyridin-7-yl)-3,4-dihydroquinolin-1(2H)-yl)pyrrolidine-1-carboxylate

To a solution of (S)-tert-butyl 3-(6-cyano-8-(2-(hydroxymethyl)thieno[3,2-b]pyridin-7-yl)-3,4-dihydroquinolin-1(2H)-yl)pyrrolidine-1-carboxylate (400 mg, 0.81 mmol) in THF (3 mL) was added 1-(2,2,2-trifluoroethyl)pyrimidine-2,4-dione (316 mg, 1.63 mmol), triphenylphosphine (427 mg, 1.63 mmol) and diisopropylazodicarboxylate (329 mg, 1.63 mmol) at 0° C. After stirred at 50° C. for 1 h, the reaction mixture was filtered and the filtrate was concentrated under reduced pressure to remove solvent. The residue was purified by column chromatography to afford the title compound. LCMS R_{T}=0.913 min, m/z=667.1 [M+H]⁺.

### Step 2: (S)-8-(2-((2,6-dioxo-3-(2,2,2-trifluoroethyl)-2,3-dihydropyrimidin-1(6H)-yl)methyl)thieno[3,2-b]pyridin-7-yl)-1-(pyrrolidin-3-yl)-1,2,3,4-tetrahydroquinoline-6-carbonitrile

To a solution of (S)-tert-butyl 3-(6-cyano-8-(2-((2,6-dioxo-3-(2,2,2-trifluoroethyl)-2,3-dihydropyrimidin-1(6H)-yl)methyl)thieno[3,2-b]pyridin-7-yl)-3,4-dihydroquinolin-1(2H)-yl)pyrrolidine-1-carboxylate (540 mg, 0.80 mmol) was added hydrochloric acid (4 M in dioxane, 5 mL) at 20° C., the mixture was stirred at 20° C. for 0.5 h. The reaction mixture was concentrated under reduced pressure to remove solvent. The residue was purified by preparative HPLC to afford the title compound. LCMS R_{T}=1.103 min, m/z=567.1 [M+H]⁺.

### Step 3

To a solution of (S)-8-(2-((2,6-dioxo-3-(2,2,2-trifluoroethyl)-2,3-dihydropyrimidin-1(6H)-yl)methyl)thieno[3,2-b]pyridin-7-yl)-1-(pyrrolidin-3-yl)-1,2,3,4-tetrahydroquinoline-6-carbonitrile (200 mg, 0.35 mmol) in acetonitrile (1 mL) was added 2-hydroxycyclobutanone (36 mg, 0.42 mmol) and sodium borohydride acetate (224 mg, 1.06 mmol) and triethylamine (107 mg, 1.06 mmol) at 20° C. After stirred for 1 h at 20° C., the reaction mixture was filtered and the filtrate was concentrated under reduced pressure to remove solvent. The residue was purified by preparative HPLC followed by SFC give first eluting fraction (Example 107a) and second eluting fraction (Example 107b).

Example 107a: ¹H NMR (400 MHz, CD₃OD) δ=1.03 (s, 1H), 1.16-1.35 (m, 1H), 1.45 (s, 1H), 1.58 (s, 1H), 1.70 (s, 1H), 1.89-2.04 (m, 3H), 2.18 (s, 1H), 2.35 (s, 1H), 2.44-2.61 (m, 2H), 2.77 (s, 1H), 2.88 (d, J=4.8 Hz, 2H), 3.61-3.90 (m, 2H), 4.57-4.79 (m, 3H), 4.79-4.85 (m, 1H), 5.39 (s, 2H), 5.87 (d, J=8.0 Hz, 1H), 7.42 (s, 1H), 7.45-7.52 (m, 2H), 7.55 (s, 1H), 7.64 (d, J=7.6 Hz, 1H), 8.72 (d, J=4.8 Hz, 1H). LCMS R_{T}=0.502 min, m/z=637.4 [M+H]+(*absolute configuration is not determined.)

Example 107b: ¹H NMR (400 MHz, CD₃OD) δ=1.00 (s, 1H), 1.11-1.28 (m, 1H), 1.22 (s, 1H), 1.28 (s, 1H), 1.26-1.39 (m, 1H), 1.46 (s, 1H), 1.58-1.74 (m, 1H), 1.74-1.92 (m, 1H), 2.01 (s, 1H), 2.21-2.44 (m, 1H), 2.53 (s, 2H), 2.61 (s, 1H), 2.87 (d, J=5.2 Hz, 2H), 3.58-3.91 (m, 2H), 4.62 (q, J=8.4 Hz, 3H), 4.81-4.83 (m, 1H), 5.38 (s, 2H), 5.88 (d, J=8.0 Hz, 1H), 7.41 (s, 1H), 7.43-7.51 (m, 2H), 7.53 (s, 1H), 7.65 (d, J=7.6 Hz, 1H), 8.71 (d, J=4.8 Hz, 1H). LCMS R_{T}=0.500 min, m/z=637.4 [M+H]⁺. (*absolute configuration is not determined.)

### Example 108a and 108b: (S)-1-((7-(6-chloro-1-(pyrrolidin-3-yl)-1,2,3,4-tetrahydroquinolin-8-yl)-3-fluorothieno[3,2-b]pyridin-2-yl)methyl)pyrrolidine-2,5-dione, formic acid salt and (R)-1-((7-(6-chloro-1-(pyrrolidin-3-yl)-1,2,3,4-tetrahydroquinolin-8-yl)-3-fluorothieno[3,2-b]pyridin-2-yl)methyl)pyrrolidine-2,5-dione, formic acid salt

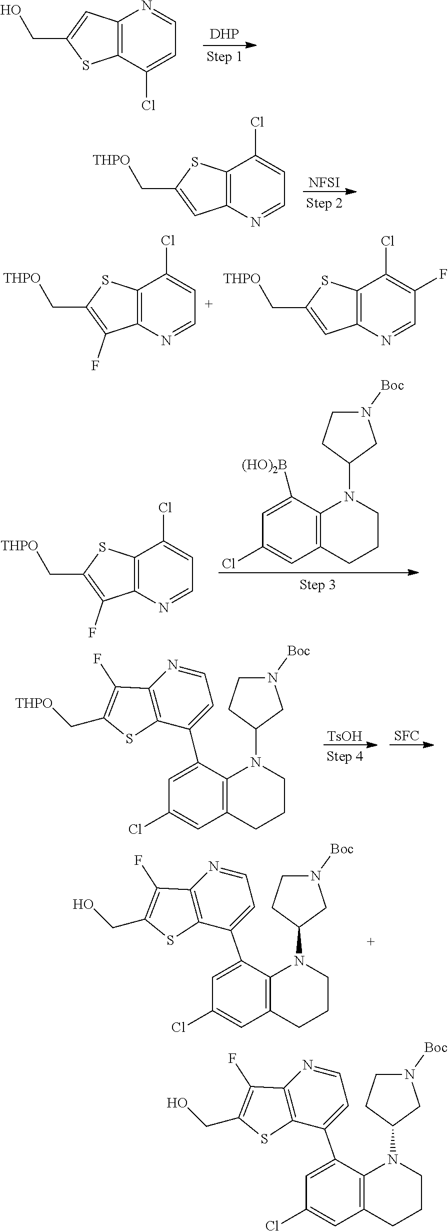

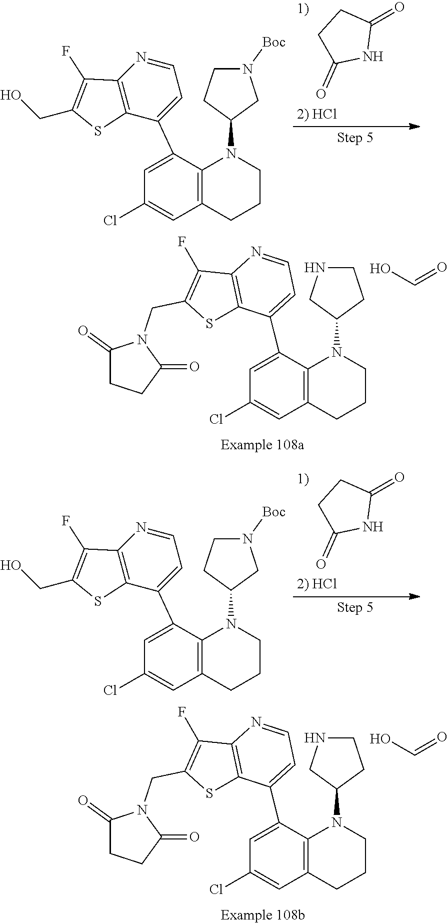

### Step 1: 7-chloro-2-(((tetrahydro-2H-pyran-2-yl)oxy)methyl)thieno[3,2-b]pyridine

To a solution of (7-chlorothieno[3,2-b]pyridin-2-yl)methanol (5.0 g, 25.04 mmol) in acetone (80 mL) was added (3,4-dihydro-2H-pyran-2-yl)methanol (6.3 g, 75.13 mmol) and pyridinium p-toluenesulfonate (2.52 g, 10.02 mmol). After stirred at 45° C. for 10 h, the mixture was quenched by addition of saturated sodium bicarbonate (200 mL) and extracted with ethyl acetate (80 mL×2). The combined organic layers were washed with brine (100 mL), dried over anhydrous sodium sulfate and concentrated under reduced pressure. The residue was purified by column chromatography to afford the title compound. LCMS R_{T}=0.943 min, m/z=284.0 [M+H]⁺.

### Step 2: 7-chloro-3-fluoro-2-(((tetrahydro-2H-pyran-2-yl)oxy)methyl)thieno[3,2-b]pyridine

To a solution of 7-chloro-2-(tetrahydropyran-2-yloxymethyl)thieno[3,2-b]pyridine (3.0 g, 10.57 mmol) in THF (30 mL) was added n-butyllithium (8.5 mL, 2.5 M in THF) at −65° C. After stirred at −65° C. for 0.5 h, the mixture was added N-(benzenesulfonyl)-N-fluorobenzenesulfonamide (4 g, 12.69 mmol). After stirred at −65° C. for 2.5 h and 15° C. for 2 h, the mixture was quenched by addition of saturated ammonium chloride (90 mL) and extracted with ethyl acetate (50 mL×3). The combined organic layers were washed with brine (70 mL), dried over anhydrous sodium sulfate and concentrated under reduced pressure. The residue was purified by column chromatography to afford the title compound. LCMS R_{T}=1.960 min, m/z=302.1 [M+H]⁺.

### Step 3: tert-butyl 3-(6-chloro-8-(3-fluoro-2-(((tetrahydro-2H-pyran-2-yl)oxy)methyl)thieno[3,2-b]pyridin-7-yl)-3,4-dihydroquinolin-1(2H)-yl)pyrrolidine-1-carboxylate

To a solution of 7-chloro-3-fluoro-2-(((tetrahydro-2H-pyran-2-yl)oxy)methyl)thieno[3,2-b]pyridine (190 mg, 0.63 mmol) and (1-(1-(tert-butoxycarbonyl)pyrrolidin-3-yl)-6-chloro-1,2,3,4-tetrahydroquinolin-8-yl)boronic acid (312 mg, 0.81 mmol) in dioxane (6 mL) and water (0.5 mL) was added potassium carbonate (218 mg, 1.57 mmol) and dichloro[1,1′-bis(diphenylphosphino)ferrocene]palladium(II) (92 mg, 0.13 mmol). After stirred at 95° C. for 3 h, the mixture was filtered and the filtrate was concentrated under reduced pressure. The residue was purified by column chromatography to afford the title compound. LCMS R_{T}=2.360 min, m/z=602.3 [M+H]⁺.

### Step 4: (S)-tert-butyl 3-(6-chloro-8-(3-fluoro-2-(hydroxymethyl)thieno[3,2-b]pyridin-7-yl)-3,4-dihydroquinolin-1(2H)-yl)pyrrolidine-1-carboxylate and (R)-tert-butyl 3-(6-chloro-8-(3-fluoro-2-(hydroxymethyl)thieno[3,2-b]pyridin-7-yl)-3,4-dihydroquinolin-1(2H)-yl)pyrrolidine-1-carboxylate

To a solution of tert-butyl 3-[6-chloro-8-[3-fluoro-2-(tetrahydropyran-2-yloxymethyl)thieno[3,2-b]pyridin-7-yl]-3,4-dihydro-2H-quinolin-1-yl]pyrrolidine-1-carboxylate (90 mg, 0.15 mmol) and tert-butyl 3-[6-chloro-8-[6-fluoro-2-(tetrahydropyran-2-yloxymethyl)thieno[3,2-b]pyridin-7-yl]-3,4-dihydro-2H-quinolin-1-yl]pyrrolidine-1-carboxylate (90 mg, 0.15 mmol) in methanol (4 mL) was added 4-methylbenzenesulfonic acid hydrate (57 mg, 0.30 mmol). After stirred at 20° C. for 3 h, the mixture was quenched by addition of saturated sodium bicarbonate (10 mL) and extracted with ethyl acetate (10 mL×2). The combined organic layers were washed with brine (10 mL), dried over anhydrous sodium sulfate and concentrated under reduced pressure. The residue was purified by pre-TLC to afford the racemate. It was separated by SFC to give first eluting fraction (S)-tert-butyl 3-(6-chloro-8-(3-fluoro-2-(hydroxymethyl)thieno[3,2-b]pyridin-7-yl)-3,4-dihydroquinolin-1(2H)-yl)pyrrolidine-1-carboxylate (Rt=1.453 min, LCMS R_{T}=3.987 min, m/z=518.1 [M+H]+) and second eluting fraction (R)-tert-butyl 3-(6-chloro-8-(3-fluoro-2-(hydroxymethyl)thieno[3,2-b]pyridin-7-yl)-3,4-dihydroquinolin-1(2H)-yl)pyrrolidine-1-carboxylate (Rt=1.612 min, LCMS R_{T}=3.987 min, m/z=518.1 [M+H]+)

### Step 5

To a solution of (S)-tert-butyl 3-(6-chloro-8-(3-fluoro-2-(hydroxymethyl)thieno[3,2-b]pyridin-7-yl)-3,4-dihydroquinolin-1(2H)-yl)pyrrolidine-1-carboxylate (20 mg, 0.04 mmol) in toluene (1.5 mL) was added succinimide (8 mg, 0.08 mmol), tetramethylazodicarboxamide (20 mg, 0.12 mmol) and tributylphosphane (39 mg, 0.19 mmol). After stirred at 100° C. for 1 h, the mixture was dissolved in hydrochloric acid (1.3 mL, 4 M in dioxane). After stirred at 20° C. for 0.5 h, the mixture was concentrated under reduced pressure. The residue was purified by preparative HPLC to afford Example 108a. ¹H NMR (400 MHz, CD₃OD) δ=8.79 (d, J=4.8 Hz, 1H), 8.51 (s, 1H), 7.59 (d, J=4.8 Hz, 1H), 7.18 (dd, J=17.2 Hz, 2.4 Hz, 2H), 4.96 (s, 2H), 3.66 (q, J=8.4 Hz, 1H), 3.29-3.08 (m, 4H), 2.89 (t, J=6.4 Hz, 2H), 2.74 (s, 6H), 1.95 (q, J=6.4 Hz, 2H), 1.89-1.43 (m, 2H). LCMS R_{T}=1.600 min, m/z=499.0 [M+H]⁺.

Example 108b was prepared from (R)-tert-butyl 3-(6-chloro-8-(3-fluoro-2-(hydroxymethyl)thieno[3,2-b]pyridin-7-yl)-3,4-dihydroquinolin-1(2H)-yl)pyrrolidine-1-carboxylate, following the procedure described in the synthesis of Example 108a. The product was purified by preparative HPLC. ¹H NMR (400 MHz, CD₃OD) δ=8.78 (d, J=4.8 Hz, 1H), 8.51 (s, 1H), 7.58 (d, J=4.8 Hz, 1H), 7.17 (dd, J=18.4 Hz, 2.4 Hz, 2H), 4.96 (s, 2H), 3.65 (q, J=8.4 Hz, 1H), 3.30-3.09 (m, 4H), 2.88 (t, J=6.4 Hz, 2H), 2.81-2.62 (m, 6H), 1.95 (q, J=6.4 Hz, 2H), 1.80 (s, 2H). LCMS R_{T}=1.620 min, m/z=499.0 [M+H]⁺. The absolute configuration was tentatively assigned based on the relative potency of the pair enantiomers in biology assays.

### Example 109: 1-((7-(6-chloro-1-((S)-pyrrolidin-3-yl)-1,2,3,4-tetrahydroquinolin-8-yl)-6-fluorothieno[3,2-b]pyridin-2-yl)methyl)pyrrolidine-2,5-dione, formic acid salt

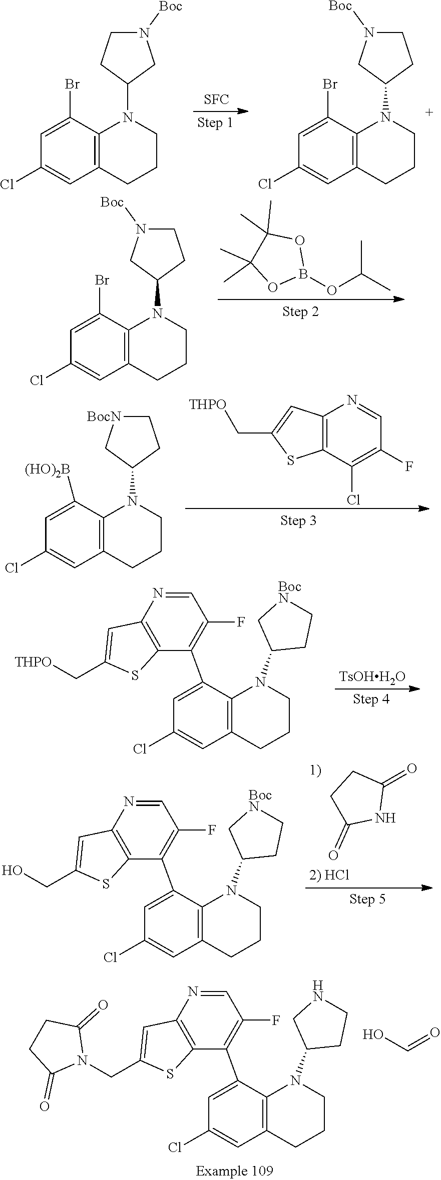

### Step 1: (S)-tert-butyl 3-(8-bromo-6-chloro-3,4-dihydroquinolin-1(2H)-yl)pyrrolidine-1-carboxylate and (R)-tert-butyl 3-(8-bromo-6-chloro-3,4-dihydroquinolin-1(2H)-yl)pyrrolidine-1-carboxylate

The tert-butyl 3-(8-bromo-6-chloro-3,4-dihydroquinolin-1(2H)-yl)pyrrolidine-1-carboxylate (10 g, 24.05 mmol) was separated by SFC to give first eluting fraction (S)-tert-butyl 3-(8-bromo-6-chloro-3,4-dihydroquinolin-1(2H)-yl)pyrrolidine-1-carboxylate (Rt=1.727 min, LCMS R_{T}=0.681 min, m/z=416.9 [M+H]+) and second eluting fraction (R)-tert-butyl 3-(8-bromo-6-chloro-3,4-dihydroquinolin-1(2H)-yl)pyrrolidine-1-carboxylate (Rt=1.934 min, LCMS R_{T}=0.681 min, m/z=416.9 [M+H]⁺.) The absolute configuration was tentatively assigned based on the relative potency of the pair enantiomers in biology assays.

### Step 2: (S)-(1-(1-(tert-butoxycarbonyl)pyrrolidin-3-yl)-6-chloro-1,2,3,4-tetrahydroquinolin-8-yl)boronic acid

To a solution of isopropylmagnesium chloride-lithium chloride (5.2 mL, 1.3 M in THF) was added (S)-tert-butyl 3-(8-bromo-6-chloro-3,4-dihydroquinolin-1(2H)-yl)pyrrolidine-1-carboxylate (400 mg, 0.96 mmol) in THF (2 mL) at 0° C. After the mixture was stirred at 0° C. for 0.5 h, 2-isopropoxy-4,4,5,5-tetramethyl-1,3,2-dioxaborolane (358 mg, 1.92 mmol) was added. After stirred at 0° C. for 0.5 h, the mixture was quenched by addition of saturated ammonium chloride (10 mL) and extracted with ethyl acetate (20 mL). The organic layer was washed with brine (20 mL), dried over anhydrous sodium sulfate and concentrated under reduced pressure to afford the crude title compound which was used in next step without further purification. LCMS R_{T}=1.488 min, m/z=381.2 [M+H]⁺.

### Step 3: (3S)-tert-butyl 3-(6-chloro-8-(6-fluoro-2-(((tetrahydro-2H-pyran-2-yl)oxy)methyl)thieno[3,2-b]pyridin-7-yl)-3,4-dihydroquinolin-1(2H)-yl)pyrrolidine-1-carboxylate

To a solution of 7-chloro-6-fluoro-2-(((tetrahydro-2H-pyran-2-yl)oxy)methyl)thieno[3,2-b]pyridine (200 mg, 0.66 mmol) and (S)-(1-(1-(tert-butoxycarbonyl)pyrrolidin-3-yl)-6-chloro-1,2,3,4-tetrahydroquinolin-8-yl)boronic acid (631 mg, 1.66 mmol) in dioxane (7 mL) and water (0.5 mL) were added cesium carbonate (648 mg, 1.99 mmol), dicyclohexyl-[2-(2,6-dimethoxyphenyl)phenyl]phosphane (109 mg, 0.27 mmol) and dichloro[1,1′-bis(diphenylphosphino)ferrocene]palladium(II) (97 mg, 0.13 mmol). After stirred at 95° C. for 3 h, the mixture was diluted with water (40 mL) and extracted with ethyl acetate (30 mL×2). The combined organic layers were washed with brine (30 mL), dried over anhydrous sodium sulfate and concentrated under reduced pressure. The residue was purified by column chromatography to afford the title compound. LCMS R_{T}=2.397 min, m/z=602.3 [M+H]⁺.

### Step 4: (3S)-tert-butyl 3-(6-chloro-8-(6-fluoro-2-(hydroxymethyl)thieno[3,2-b]pyridin-7-yl)-3,4-dihydroquinolin-1(2H)-yl)pyrrolidine-1-carboxylate

To a solution of (3S)-tert-butyl 3-(6-chloro-8-(6-fluoro-2-(((tetrahydro-2H-pyran-2-yl)oxy)methyl)thieno[3,2-b]pyridin-7-yl)-3,4-dihydroquinolin-1(2H)-yl)pyrrolidine-1-carboxylate (120 mg, 0.20 mmol) in methanol (4 mL) was added 4-methylbenzenesulfonic acid hydrate (42 mg, 0.22 mmol). After stirred at 20° C. for 2 h, the mixture was quenched by addition of saturated sodium bicarbonate (30 mL) and extracted with ethyl acetate (20 mL×2). The combined organic layers were washed with brine (20 mL), dried over anhydrous sodium sulfate and concentrated under reduced pressure. The residue was purified by pre-TLC to afford the title compound. LCMS R_{T}=1.050 min, m/z=518.2 [M+H]⁺.

### Step 5

Example 109 was prepared from (3S)-tert-butyl 3-(6-chloro-8-(6-fluoro-2-(hydroxymethyl)thieno[3,2-b]pyridin-7-yl)-3,4-dihydroquinolin-1(2H)-yl)pyrrolidine-1-carboxylate, following the procedure described in the synthesis of Example 108a. The product was purified by preparative HPLC. ¹H NMR (400 MHz, CD₃OD) δ=8.68 (dd, J=2.0, 12.8 Hz, 1H), 8.53 (s, 1H), 7.46 (d, J=4.4 Hz, 1H), 7.24-7.13 (m, 2H), 4.94-4.90 (m, 2H), 3.65-3.55 (m, 1H), 3.29-3.10 (m, 3H), 3.08-2.86 (m, 3H), 2.75 (s, 4H), 2.72-2.62 (m, 1H), 2.48-1.90 (m, 3H), 1.77-1.67 (m, 1H), 1.60-0.81 (m, 1H).

### Example 110: (S)-1-(1-(7-(6-chloro-1-(pyrrolidin-3-yl)-1,2,3,4-tetrahydroquinolin-8-yl)benzo[b]thiophen-2-yl)cyclopropyl)pyrrolidine-2,5-dione, formic acid salt

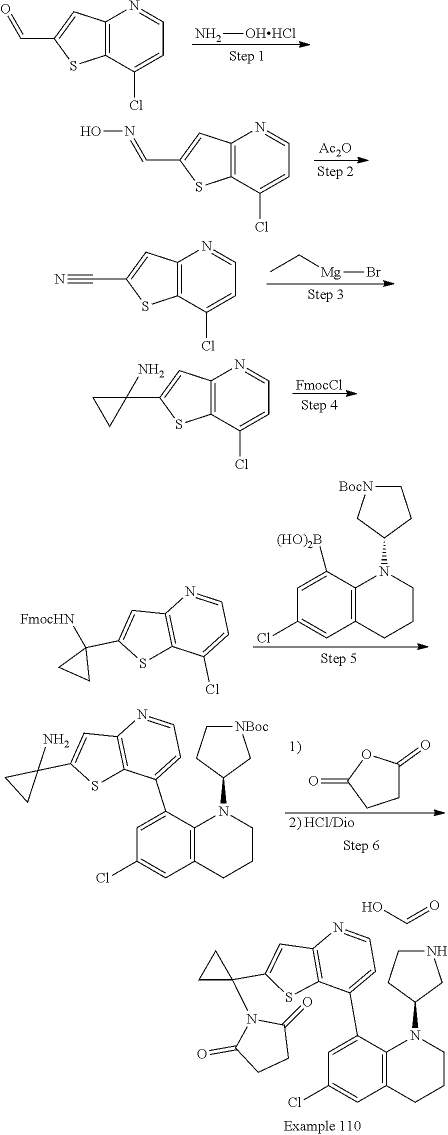

### Step 1: (E)-7-chlorothieno[3,2-b]pyridine-2-carbaldehyde oxime

To a solution of 7-chlorothieno[3,2-b]pyridine-2-carbaldehyde (15.0 g, 75.90 mmol) in methanol (220 mL) was added hydroxylammonium chloride (5.8 g, 83.48 mmol). After stirred at 20° C. for 0.5 h, the mixture was concentrated under reduced pressure. The residue was diluted with water (10 mL) and the mixture was filtered. The filtrate was concentrated under reduced pressure to afford the crude title compound, which was used in next step without further purification. LCMS R_{T}=0.783 min, m/z=212.9 [M+H]⁺.

### Step 2: 7-chlorothieno[3,2-b]pyridine-2-carbonitrile

To a solution of 7-chlorothieno[3,2-b]pyridine-2-carbaldehyde oxime (15.0 g, 70.54 mmol) in acetic oxide (200 mL). After stirred at 100° C. for 16 h, the mixture was diluted with water (300 mL) and extracted with ethyl acetate (300 mL×2). The combined organic layers were washed with brine (200 mL), dried over anhydrous sodium sulfate and concentrated under reduced pressure. The residue was purified by column chromatography to give the title compound. LCMS R_{T}=0.887 min, m/z=195.0 [M+H]⁺.

### Step 3: 1-(7-chlorothieno[3,2-b]pyridin-2-yl)cyclopropanamine

To a solution of 7-chlorothieno[3,2-b]pyridine-2-carbonitrile (2.0 g, 10.28 mmol) in dioxane (40 mL) was added titanium tetraisopropanolate (23.2 g, 11.30 mol) and ethylmagnesium bromide (7.9 mL, 3 M in THF). After stirred at 20° C. for 1.5 h, the mixture was added (diethyloxonio)trifluoroborate (2.9 g, 20.55 mmol). After stirred at 20° C. for 1 h, the mixture was quenched by addition of sodium hydroxide (15 mL, 2 M in water). The mixture was filtered and the filtrate was diluted with water (40 mL), extracted with ethyl acetate (30 mL×4). The combined organic layers were washed with brine (50 mL), dried over anhydrous sodium sulfate and concentrated under reduced pressure. The residue was purified by column chromatography to afford the title compound. LCMS R_{T}=0.687 min, m/z=225.0 [M+H]⁺.

### Step 4: (9H-fluoren-9-yl)methyl (1-(7-chlorothieno[3,2-b]pyridin-2-yl)cyclopropyl)carbamate

To a solution of 1-(7-chlorothieno[3,2-b]pyridin-2-yl)cyclopropanamine (500 mg, 2.23 mmol) in dioxane (8 mL) and water (8 mL) was added sodium carbonate (354 mg, 3.34 mol) and 9H-fluoren-9-ylmethyl carbonochloridate (863 mg, 3.34 mmol) at 0° C. After stirred at 20° C. for 2 h, the mixture was diluted with water (15 mL) and extracted with ethyl acetate (15 mL×2). The combined organic layers were washed with brine (15 mL), dried over anhydrous sodium sulfate and concentrated under reduced pressure. The residue was purified by column chromatography to afford the title compound. LCMS R_{T}=0.517 min, m/z=446.9 [M+H]⁺.

### Step 5: (S)-tert-butyl 3-(8-(2-(1-((((9H-fluoren-9-yl)methoxy)carbonyl)amino)cyclopropyl)thieno[3,2-b]pyridin-7-yl)-6-chloro-3,4-dihydroquinolin-1(2H)-yl)pyrrolidine-1-carboxylate

To a solution of 9H-fluoren-9-ylmethyl N-[1-(7-chlorothieno[3,2-b]pyridin-2-yl)cyclopropyl]carbamate (250 mg, 0.56 mmol) and [1-[(3S)-1-tert-butoxycarbonylpyrrolidin-3-yl]-6-chloro-3,4-dihydro-2H-quinolin-8-yl]boronic acid (319 mg, 0.84 mmol) in dioxane (7 mL) and water (0.5 mL) was added cesium carbonate (456 mg, 1.40 mmol), dicyclohexyl-[2-(2,6-dimethoxyphenyl)phenyl]phosphane (92 mg, 0.22 mmol) and dichloro[1,1′-bis(diphenylphosphino)ferrocene]palladium(II) (82 mg, 0.11 mmol). After stirred at 100° C. for 3 h, the mixture was concentrated under reduced pressure. The residue was purified by column chromatography to afford the title compound. LCMS R_{T}=1.392 min, m/z=525.2 [M+H]⁺.

### Step 6

To a solution of (S)-tert-butyl 3-(8-(2-(1-((((9H-fluoren-9-yl)methoxy)carbonyl)amino)cyclopropyl)thieno[3,2-b]pyridin-7-yl)-6-chloro-3,4-dihydroquinolin-1(2H)-yl)pyrrolidine-1-carboxylate (60 mg, 0.11 mmol) in toluene (1.5 mL) were added triethylamine (23 mg, 0.23 mmol) and dihydrofuran-2,5-dione (14 mg, 0.14 mmol). After stirred at 100° C. for 14 h, the mixture was dissolved in hydrochloric acid (4 mL, 4 M in dioxane). After stirred at 20° C. for 0.5 h, the mixture was concentrated under reduced pressure. The residue was purified by preparative HPLC to afford Example 110. ¹H NMR (400 MHz, CD₃OD) δ=8.68 (d, J=4.8 Hz, 1H), 8.50 (s, 1H), 7.44 (d, J=4.8 Hz, 1H), 7.29 (s, 1H), 7.19-7.13 (m, 2H), 3.66 (q, J=8.4 Hz, 1H), 3.29-3.10 (m, 3H), 2.88 (t, J=6.4 Hz, 2H), 2.73 (s, 5H), 1.94 (q, J=6.4 Hz, 2H), 1.60 (s, 4H). LCMS R_{T}=0.443 min, m/z=507.1 [M+H]⁺.

### Example 111a and 111b: (S)-1-((7-(4-(pyrrolidin-3-yl)-3,4-dihydro-2H-benzo[b][1,4]oxazin-5-yl)thieno[3,2-b]pyridin-2-yl)methyl)pyrrolidine-2,5-dione, formic acid salt and (R)-1-((7-(4-(pyrrolidin-3-yl)-3,4-dihydro-2H-benzo[b][1,4]oxazin-5-yl)thieno[3,2-b]pyridin-2-yl)methyl)pyrrolidine-2,5-dione, formic acid salt

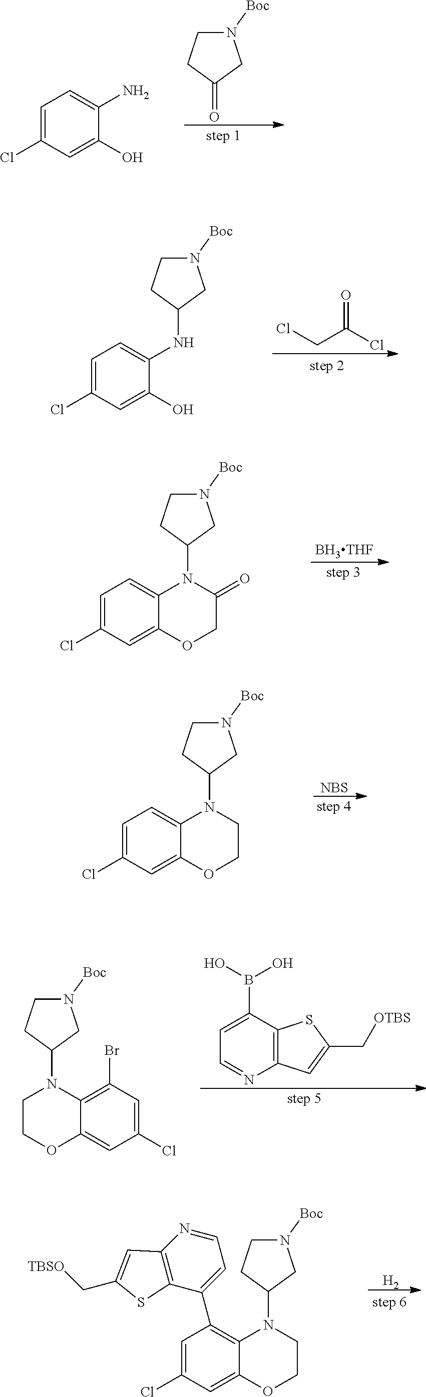

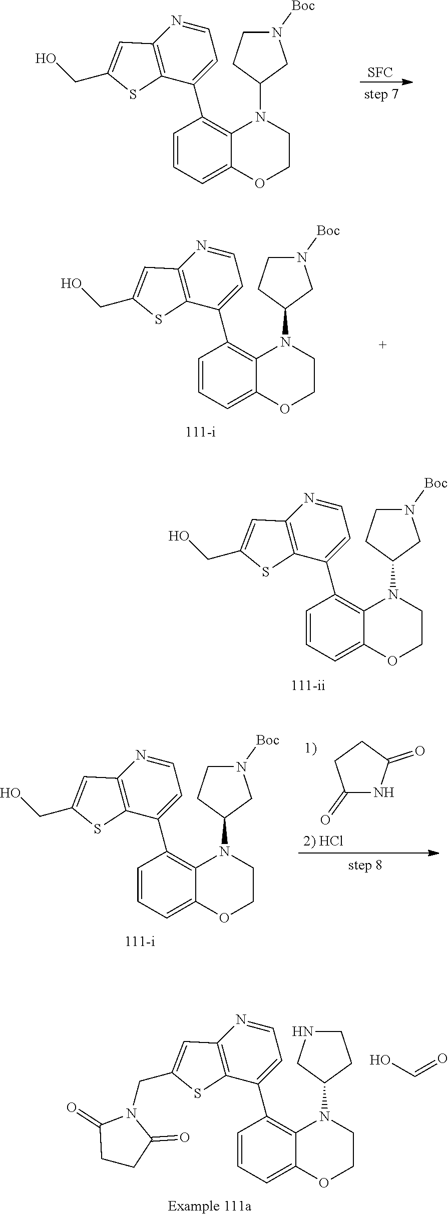

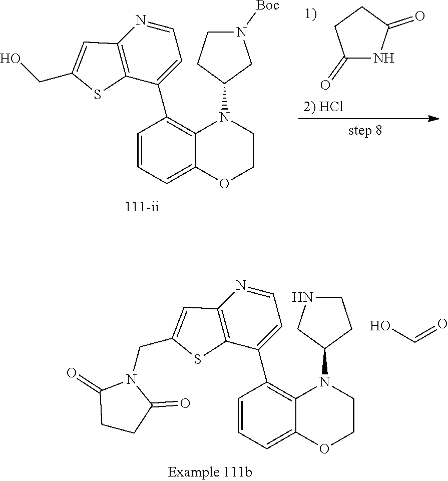

### Step 1: tert-butyl 3-((4-chloro-2-hydroxyphenyl)amino)pyrrolidine-1-carboxylate

To a solution of 2-amino-5-chloro-phenol (10.0 g, 69.65 mmol) in 1,2-dichloroethane (200 mL) was added acetic acid (2.1 g, 34.83 mmol, 1.99 mL), tert-butyl 3-oxopyrrolidine-1-carboxylate (14.2 g, 76.62 mmol) and sodium triacetoxyborohydride (29.5 g, 139.30 mmol). After stirred at 25° C. for 1 h, the mixture was concentrated. The residue was diluted with water (200 mL) and extracted with ethyl acetate (150 mL×3). The combined organic layers were washed with brine (250 mL), dried over anhydrous sodium sulfate and concentrated under reduced pressure. The residue was purified by column chromatography to afford the title compound. LCMS R_{T}=0.555 min, m/z=257.1 [M+H−56]⁺.

### Step 2: tert-butyl 3-(7-chloro-3-oxo-2H-benzo[b][1,4]oxazin-4(3H)-yl)pyrrolidine-1-carboxylate

To a solution of tert-butyl 3-((4-chloro-2-hydroxyphenyl)amino)pyrrolidine-1-carboxylate (5.0 g, 15.99 mmol) in acetonitrile (30 mL) was added potassium carbonate (16 mL, 3 M in water) and 2-chloroacetyl chloride (3.61 g, 31.97 mmol). After stirred at 60° C. for 8 h, the mixture was diluted with saturated sodium bicarbonate (20 mL) and extracted with ethyl acetate (30 mL×3). The combined organic layers were washed with brine (30 mL), dried over anhydrous sodium sulfate, filtered and the filtrate was concentrated under reduced pressure. The residue was purified by column chromatography to afford the title compound. LCMS R_{T}=0.983 min, m/z=297.8 [M+H−56]⁺.

### Step 3: tert-butyl 3-(7-chloro-2H-benzo[b][1,4]oxazin-4(3H)-yl)pyrrolidine-1-carboxylate

To a solution of tert-butyl 3-(7-chloro-3-oxo-2H-benzo[b][1,4]oxazin-4(3H)-yl)pyrrolidine-1-carboxylate (2.5 g, 7.09 mmol) in THF (10 mL) was added borane (25 mL, 1 M in THF) at 0° C. After stirred at 20° C. for 1 h, the mixture was quenched by addition of methanol (50 mL) and concentrated under reduced pressure. The residue was purified by column chromatography to afford the title compound. LCMS R_{T}=1.778 min, m/z=283.2 [M+H−56]⁺.

### Step 4: tert-butyl 3-(5-bromo-7-chloro-2H-benzo[b][1,4]oxazin-4(3H)-yl)pyrrolidine-1-carboxylate

To a solution of tert-butyl 3-(7-chloro-2H-benzo[b][1,4]oxazin-4(3H)-yl)pyrrolidine-1-carboxylate (1.7 g, 5.02 mmol) in DMF (1 mL) was added N-bromosuccinimide (893 mg, 5.02 mmol) at 0° C. After stirred at 20° C. for 1 h, the mixture was quenched by addition of water (100 mL) and extracted with ethyl acetate (50 mL×3). The combined organic layers were dried over anhydrous sodium sulfate, filtered and the filtrate was concentrated under reduced pressure. The residue was purified by column chromatography to afford the title compound. LCMS R_{T}=1.940 min, m/z=363.0 [M+H−56]⁺.

### Step 5: tert-butyl 3-(5-(2-(((tert-butyldimethylsilyl)oxy)methyl)thieno[3,2-b]pyridin-7-yl)-7-chloro-2H-benzo[b][1,4]oxazin-4(3H)-yl)pyrrolidine-1-carboxylate

To a solution of tert-butyl 3-(5-bromo-7-chloro-2H-benzo[b][1,4]oxazin-4(3H)-yl)pyrrolidine-1-carboxylate (2.0 g, 4.79 mmol) in dioxane (20 mL) and water (1 mL) was added [2-[[tert-butyl(dimethyl)silyl]oxymethyl]thieno[3,2-b]pyridin-7-yl]boronic acid (2.3 g, 7.18 mmol), potassium carbonate (2.0 g, 14.36 mmol) and dichloro[1,1′-bis(diphenylphosphino)ferrocene]palladium(II) (350 mg, 4.79 mmol). After stirred at 100° C. for 2 h under nitrogen atmosphere, the mixture was filtered and the filtrate was concentrated under reduced pressure. The residue was purified by column chromatography to afford the title compound. LCMS R_{T}=1.143 min, m/z=616.2 [M+H]⁺.

### Step 6: tert-butyl 3-(5-(2-(hydroxymethyl)thieno[3,2-b]pyridin-7-yl)-2H-benzo[b][1,4]oxazin-4(3H)-yl)pyrrolidine-1-carboxylate

To a solution of tert-butyl 3-(5-(2-(((tert-butyldimethylsilyl)oxy)methyl)thieno[3,2-b]pyridin-7-yl)-7-chloro-2H-benzo[b][1,4]oxazin-4(3H)-yl)pyrrolidine-1-carboxylate (0.5 g, 8.11 mmol) in methanol (20 mL) was added 10% palladium on carbon (0.2 g, 1.89 mmol). After stirred at 75° C. for 16 h under hydrogen atmosphere (50 psi), the mixture was concentrated under reduced pressure. The residue was purified by preparative HPLC to afford the title compound. LCMS R_{T}=1.696 min, m/z=468.0 [M+H]⁺.

### Step 7: (R)-tert-butyl 3-(5-(2-(hydroxymethyl)thieno[3,2-b]pyridin-7-yl)-2H-benzo[b][1,4]oxazin-4(3H)-yl)pyrrolidine-1-carboxylate and (S)-tert-butyl 3-(5-(2-(hydroxymethyl)thieno[3,2-b]pyridin-7-yl)-2H-benzo[b][1,4]oxazin-4(3H)-yl)pyrrolidine-1-carboxylate

The racemate was separated by SFC to afford first eluting fraction 111-i (Rt=1.536 min, LCMS R_{T}=0.482 min, m/z=468.2 [M+H]+) and second eluting fraction 111-ii (Rt=1.729 min, LCMS R_{T}-0.482 min, m/z=468.2 [M+H]+).

### Step 8

Example 111a was prepared from 111-i, following the procedure described in the synthesis of Example 108a. The final product was purified by preparative HPLC. ¹H NMR (400 MHz, CD30D) 6=8.75 (d, J=5.2 Hz, 1H), 8.53 (s, 1H), 7.60-7.38 (m, 2H), 7.21 (s, 3H), 4.97 (s, 2H), 4.07-3.98 (m, 1H), 3.50 (s, 2H), 3.47-3.22 (m, 4H) 2.93 (t, J=6.4 Hz, 2H), 2.77 (s, 4H), 1.93 (s, 2H). LCMS R_{T}=1.550 min, m/z=467.2 [M+H+18]⁺.

Example 111b was prepared from 111-ii, following the procedure described in the synthesis of Example 108a. The final product was purified by preparative HPLC to afford the title compound. ¹H NMR (400 MHz, CD30D) 6=8.75 (d, J=5.2 Hz, 1H), 8.53 (s, 1H), 7.60-7.38 (m, 2H), 7.21 (s, 3H), 4.97 (s, 2H), 4.07-3.98 (m, 1H), 3.50 (s, 2H), 3.47-3.22 (m, 4H) 2.93 (t, J=6.4 Hz, 2H), 2.77 (s, 4H), 1.93 (s, 2H). LCMS R_{T}=1.550 min, m/z=467.2 [M+H]⁺. The absolute configuration was tentatively assigned based on the relative potency of the pair enantiomers in biology assays.

### Example 112a, 112b, 112c and 112d: 8-(2-((R)-1-(2,5-dioxopyrrolidin-1-yl)ethyl)thieno[3,2-b]pyridin-7-yl)-1-((S)-pyrrolidin-3-yl)-1,2,3,4-tetrahydroquinoline-6-carbonitrile, formic acid salt 8-(2-((S)-1-(2,5-dioxopyrrolidin-1-yl)ethyl)thieno[3,2-b]pyridin-7-yl)-1-((S)-pyrrolidin-3-yl)-1,2,3,4-tetrahydroquinoline-6-carbonitrile, formic acid salt 8-(2-((R)-1-(2,5-dioxopyrrolidin-1-yl)ethyl)thieno[3,2-b]pyridin-7-yl)-1-((R)-pyrrolidin-3-yl)-1,2,3,4-tetrahydroquinoline-6-carbonitrile, formic acid salt and 8-(2-((S)-1-(2,5-dioxopyrrolidin-1-yl)ethyl)thieno[3,2-b]pyridin-7-yl)-1-((R)-pyrrolidin-3-yl)-1,2,3,4-tetrahydroquinoline-6-carbonitrile, formic acid salt

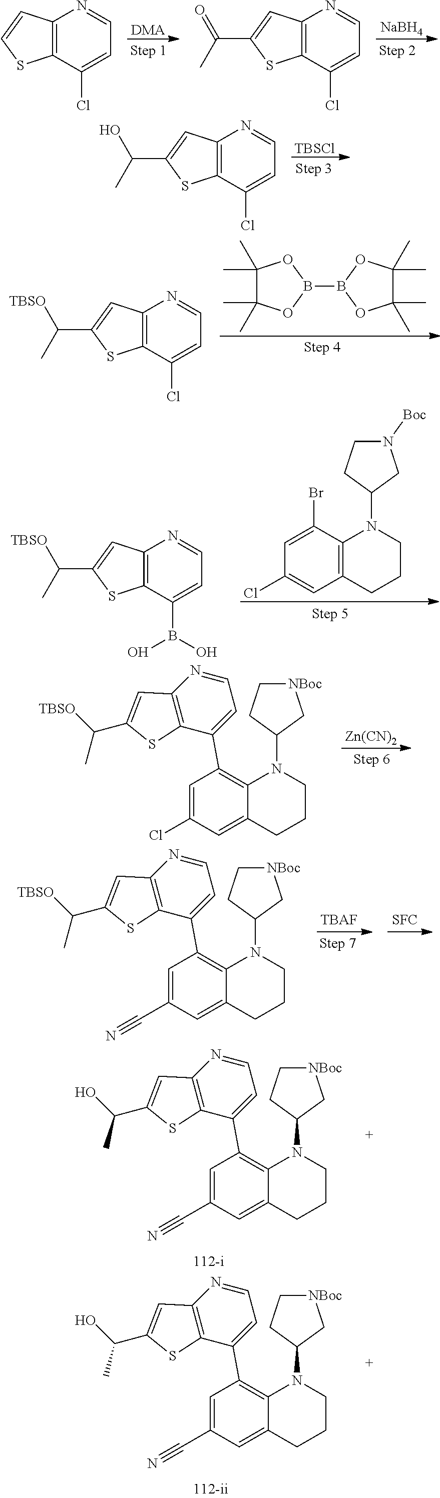

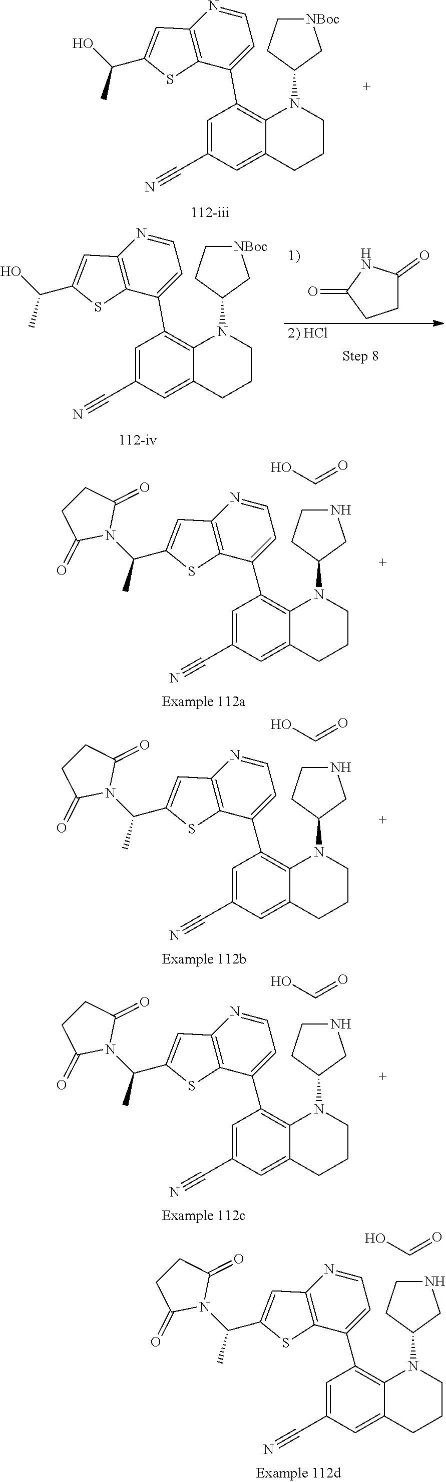

### Step 1: 1-(7-chlorothieno[3,2-b]pyridin-2-yl)ethanone

To a solution of 7-chlorothieno[3,2-b]pyridine (20.0 g, 1.18 mol) in THF (200 mL) was added n-butyllithium (70.7 mL, 2.5 M in THF) dropwise at −78° C. After stirred at −78° C. for 15 min, dimethylacetamide (25.7 g, 27.4 mL) was added dropwise at −78° C. After stirred at 0° C. for 2 h, the mixture was quenched by addition of saturated aqueous ammonium chloride (100 mL), filtered and the filtrate cake was concentrated under reduced pressure to afford the title compound, which was used in next step without further purification. LCMS R_{T}=0.857 min, m/z=212.0 [M+H]⁺.

### Step 2: 1-(7-chlorothieno[3,2-b]pyridin-2-yl)ethanol

To a solution of 1-(7-chlorothieno[3,2-b]pyridin-2-yl)ethanone (10.0 g, 47.24 mmol) in methanol (100 mL) was added sodium borohydride (9.5 g, 0.25 mol) slowly at 0° C. After stirred at 25° C. for 1 h, the mixture was quenched by addition of saturated aqueous ammonium chloride (100 mL). The mixture was filtered and the filtrate cake was concentrated under reduced pressure to afford the title compound, which was used in next step without further purification. LCMS R_{T}=1.035 min, m/z=214.2 [M+H]⁺.

### Step 3: 2-(1-((tert-butyldimethylsilyl)oxy)ethyl)-7-chlorothieno[3,2-b]pyridine

To a solution of 1-(7-chlorothieno[3,2-b]pyridin-2-yl)ethanol (6.0 g, 28 mmol) in dichloromethane (60 mL) was added imidazole (5.7 g, 84 mmol) and tert-butyl-chloro-dimethyl-silane (10.6 g, 70.2 mmol). After stirred at 25° C. for 2 h, the mixture was diluted with water (100 mL) and extracted with dichloromethane (30 mL×3). The combined organic layers were washed with brine (30 mL), dried over anhydrous sodium sulfate, filtered and the filtrate was concentrated under reduced pressure. The residue was purified by column chromatography to afford the title compound. LCMS R_{T}=0.685 min, m/z=327.8 [M+H]⁺.

### Step 4: (2-(1-((tert-butyldimethylsilyl)oxy)ethyl)thieno[3,2-b]pyridin-7-yl)boronic acid

To a solution of 2-(1-((tert-butyldimethylsilyl)oxy)ethyl)-7-chlorothieno[3,2-b]pyridine (2 g, 6.1 mmol), 4,4,5,5-tetramethyl-2-(4,4,5,5-tetramethyl-1,3,2-dioxaborolan-2-yl)-1,3,2-dioxaborolane (3.87 g, 15.25 mmol) and potassium acetate (1.80 g, 18.3 mmol) in dioxane (2 mL) was added dichloro[1,1′-bis(diphenylphosphino)ferrocene]palladium(II) (892 mg, 1.22 mmol)) under nitrogen atmosphere. After stirred at 140° C. for 1 h under microwave, the mixture was filtered and the filtrate was concentrated under reduced pressure to afford the title compound, which was used in next step without further purification. LCMS R_{T}=0.857 min, m/z=338.1 [M+H]⁺.

### Step 5: tert-butyl 3-(8-(2-(1-((tert-butyldimethylsilyl)oxy)ethyl)thieno[3,2-b]pyridin-7-yl)-6-chloro-3,4-dihydroquinolin-1(2H)-yl)pyrrolidine-1-carboxylate

To a solution of tert-butyl 3-(8-bromo-6-chloro-3,4-dihydro-2H-quinolin-1-yl)pyrrolidine-1-carboxylate (2 g, 4.81 mmol), 2-(1-((tert-butyldimethylsilyl)oxy)ethyl)-7-chlorothieno[3,2-b]pyridine (2.1 g, 6.23 mmol) and cesium carbonate (3.9 g, 12 mmol) in dioxane (20 mL) and water (3 mL) was added dichloro[1,1′-bis(diphenylphosphino)ferrocene]palladium(II) (0.53 g, 0.72 mmol) under nitrogen atmosphere. After stirred at 100° C. for 3 h, the mixture was diluted with water (30 mL) and extracted with ethyl acetate (30 mL×2). The combined organic layers were washed with brine (30 mL), dried over anhydrous sodium sulfate, filtered and the filtrate was concentrated under reduced pressure. The residue was purified by column chromatography to afford the title compound. LCMS R_{T}=0.658 min, m/z=630.0 [M+H]⁺.

### Step 6: tert-butyl 3-(8-(2-(1-((tert-butyldimethylsilyl)oxy)ethyl)thieno[3,2-b]pyridin-7-yl)-6-cyano-3,4-dihydroquinolin-1(2H)-yl)pyrrolidine-1-carboxylate

To a solution of tert-butyl 3-(8-(2-(1-((tert-butyldimethylsilyl)oxy)ethyl)thieno[3,2-b]pyridin-7-yl)-6-chloro-3,4-dihydroquinolin-1(2H)-yl)pyrrolidine-1-carboxylate (3 g, 4.77 mmol) in 1-methyl-2-pyrrilifinone (30 mL) was added zinc cyanide (1.68 g, 14.32 mmol) and bis(tri-tert-butylphosphine)palladiun (0.73 g, 1.43 mmol) under nitrogen atmosphere. After stirred at 165° C. for 1.5 h under microwave, the mixture was filtered. The filtrate was diluted with water (30 mL) and extracted with dichloromethane (30 mL×2). The combined organic layers were washed with brine (20 mL), dried over anhydrous sodium sulfate, filtered and the filtrate was concentrated under reduced pressure. The residue was purified by column chromatography to afford the title compound. LCMS R_{T}=1.100 min, m/z=619.7 [M+H]⁺.

### Step 7: (S)-tert-butyl 3-(6-cyano-8-(2-((R)-1-hydroxyethyl)thieno[3,2-b]pyridin-7-yl)-3,4-dihydroquinolin-1(2H)-yl)pyrrolidine-1-carboxylate, (S)-tert-butyl 3-(6-cyano-8-(2-((S)-1-hydroxyethyl)thieno[3,2-b]pyridin-7-yl)-3,4-dihydroquinolin-1(2H)-yl)pyrrolidine-1-carboxylate, (R)-tert-butyl 3-(6-cyano-8-(2-((R)-1-hydroxyethyl)thieno[3,2-b]pyridin-7-yl)-3,4-dihydroquinolin-1(2H)-yl)pyrrolidine-1-carboxylate and (R)-tert-butyl 3-(6-cyano-8-(2-((S)-1-hydroxyethyl)thieno[3,2-b]pyridin-7-yl)-3,4-dihydroquinolin-1(2H)-yl)pyrrolidine-1-carboxylate

To a solution of tert-butyl 3-(8-(2-(1-((tert-butyldimethylsilyl)oxy)ethyl)thieno[3,2-b]pyridin-7-yl)-6-cyano-3,4-dihydroquinolin-1(2H)-yl)pyrrolidine-1-carboxylate (3 g, 4.85 mmol) in THF (30 mL) was added tetrabutylammonium fluoride (4.85 mL, 1.0 M in THF). After stirred at 25° C. for 30 min, the mixture was concentrated under reduced pressure. The residue was purified by column chromatography. The racemate was separated by SFC to afford first eluting fraction 112-i (Rt=3.593 min, LCMS R_{T}=0.495 min, m/z=505.3 [M+H]⁺), second eluting fraction 112-ii (Rt=3.768 min, LCMS R_{T}=0.495 min, m/z=505.3 [M+H]⁺.), third eluting fraction 112-iii (Rt=3.946 min, LCMS R_{T}=0.495 min, m/z=505.3 [M+H]⁺.) and fourth eluting fraction 112-iv (Rt=4.052 min, LCMS R_{T}=0.495 min, m/z=505.3 [M+H]⁺.

### Step 8

Example 112a was prepared from 112-i, following the procedure described in the synthesis of Example 108a. The final products were purified by preparative HPLC. ¹H NMR (400 MHz, CD₃OD) δ=8.74 (s, 1H), 7.57-7.38 (m, 4H), 5.66 (s, 1H), 3.83 (t, J=8.0 Hz, 1H), 3.29-3.02 (m, 3H), 2.95-2.84 (m, 2H), 2.73 (s, 6H), 2.06-1.92 (m, 3H), 1.89-1.45 (m, 5H). LCMS R_{T}=1.121 min, m/z=486.4 [M+H]⁺.

Example 112b was prepared from 112-ii, following the procedure described in the synthesis of Example 108a. ¹H NMR (400 MHz, CD₃OD) δ=8.49 (s, 1H), 7.48 (d, J=1.2 Hz, 4H), 5.70 (m, J=6.8 Hz, 1H), 3.85 (s, 1H), 3.29-3.15 (m, 2H), 3.02-2.49 (m, 9H), 2.12-1.45 (m, 8H). LCMS R_{T}=1.121 min, m/z=486.4 [M+H]⁺.

Example 112c was prepared from 112-iii, following the procedure described in the synthesis of Example 108a. ¹H NMR (400 MHz, CD₃OD) δ=8.53 (s, 1H), 7.56-7.48 (m, 4H), 5.70 (s, 1H), 3.84 (m, J=8.4 Hz, 1H), 3.31-3.21 (m, 2H), 2.94 (s, 3H), 2.75 (s, 5H), 2.01 (s, 3H), 1.90 (d, J=7.2 Hz, 4H), 1.82-1.40 (m, 2H). LCMS R_{T}=1.118 min, m/z=486.4 [M+H]⁺.

Example 112d was prepared from 112-iv, following the procedure described in the synthesis of Example 108a. ¹H NMR (400 MHz, CD₃OD) δ=8.75 (d, J=4.0 Hz, 1H), 7.61-7.37 (m, 4H), 5.78-5.64 (m, 1H), 3.85 (d, J=6.4 Hz, 1H), 3.30 (s, 3H), 2.93 (s, 2H), 2.78-2.47 (m, 6H), 2.02 (d, J=11.2 Hz, 3H), 1.88 (d, J=7.2 Hz, 5H). LCMS R_{T}=1.121 min, m/z=486.4 [M+H]⁺.

The absolute configuration was tentatively assigned based on the relative potency of the pair diastereomers in biology assays.

### Example 113a, 113b, 113c and 113d: 1-((7-((1aS,7bR)-6-chloro-3-((R)-pyrrolidin-3-yl)-1a,2,3,7b-tetrahydro-1H-cyclopropa[c]quinolin-4-yl)thieno[3,2-b]pyridin-2-yl)methyl)pyrrolidine-2,5-dione, formic acid salt, 1-((7-((1aR,7bS)-6-chloro-3-((R)-pyrrolidin-3-yl)-1a,2,3,7b-tetrahydro-1H-cyclopropa[c]quinolin-4-yl)thieno[3,2-b]pyridin-2-yl)methyl)pyrrolidine-2,5-dione, formic acid salt, 1-((7-((1aR,7bS)-6-chloro-3-((R)-pyrrolidin-3-yl)-1a,2,3,7b-tetrahydro-1H-cyclopropa[c]quinolin-4-yl)thieno[3,2-b]pyridin-2-yl)methyl)pyrrolidine-2,5-dione, formic acid salt, and 1-((7-((1aS,7bR)-6-chloro-3-((S)-pyrrolidin-3-yl)-1a,2,3,7b-tetrahydro-1H-cyclopropa[c]quinolin-4-yl)thieno[3,2-b]pyridin-2-yl)methyl)pyrrolidine-2,5-dione, formic acid salt

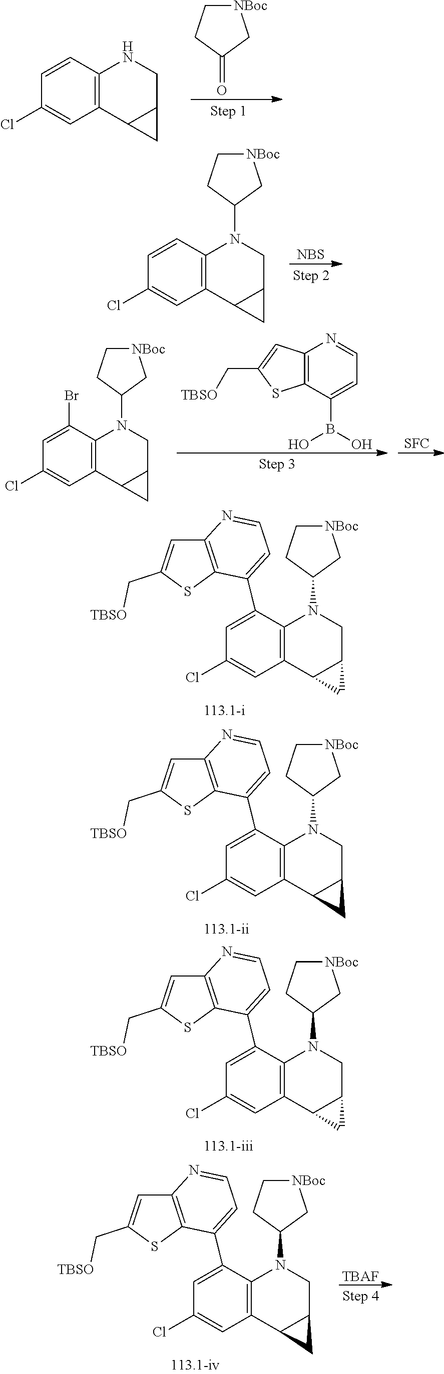

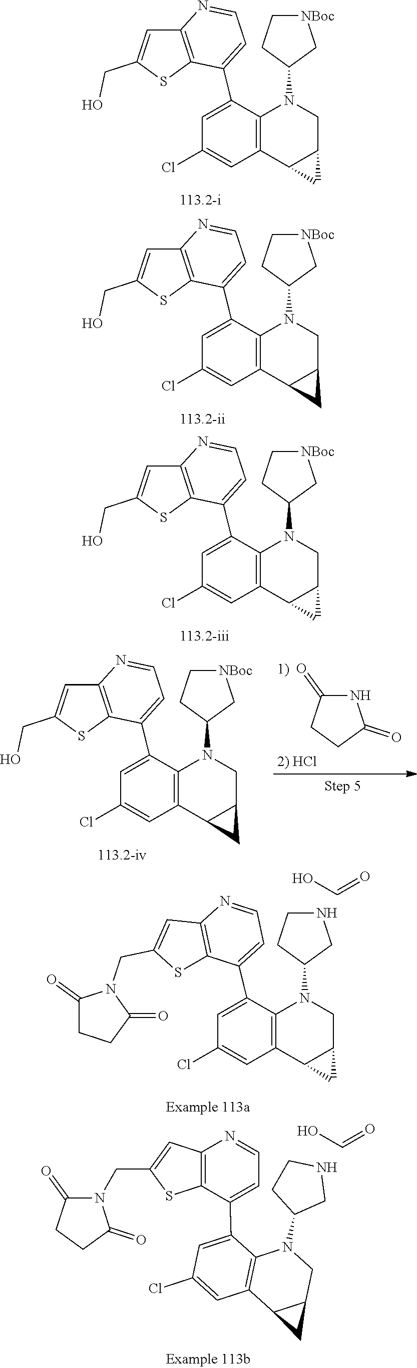

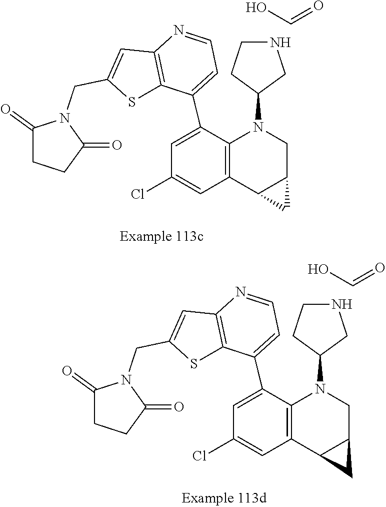

### Step 1: tert-butyl 3-(6-chloro-1a,2-dihydro-1H-cyclopropa[c]quinolin-3(7bH)-yl)pyrrolidine-1-carboxylate

To a solution of 6-chloro-1a,2,3,7b-tetrahydro-1H-cyclopropa[c]quinolone (2.4 g, 11.36 mmol) and tert-butyl 3-oxopyrrolidine-1-carboxylate (4.21 g, 22.71 mmol) in acetic acid (60 mL) was added sodium triacetoxyborohydride (4.81 g, 22.71 mmol). After stirred at 20° C. at 1.5 h, the mixture was concentrated. Then the residue was diluted with water (100 mL) and extracted with ethyl acetate (70 mL×2). The combined organic layers were washed with brine (80 mL), dried over anhydrous sodium sulfate and concentrated under reduced pressure. The residue was purified by column chromatography to afford the title compound. ¹H NMR (400 MHz, CD₃OD) δ=7.11 (d, J=2.4 Hz, 1H), 6.96 (dd, J=8.4 Hz, 2.4 Hz, 1H), 6.64 (d, J=8.4 Hz, 1H), 4.34 (d, J=6.4 Hz, 1H), 3.63-3.46 (m, 2H), 3.44-3.34 (m, 2H), 3.25 (dd, J=6.4, 11.2 Hz, 1H), 2.82-2.71 (m, 1H), 2.19-1.96 (m, 2H), 1.87 (dd, J=4.8, 8.0 Hz, 2H), 1.52-1.42 (m, 9H), 1.32-1.26 (m, 1H), 0.80 (dt, J=4.0, 8.0 Hz, 1H).

### Step 2: tert-butyl 3-(4-bromo-6-chloro-1a,2-dihydro-1H-cyclopropa[c]quinolin-3(7bH)-yl)pyrrolidine-1-carboxylate

To a solution of tert-butyl 3-(6-chloro-1a,2-dihydro-1H-cyclopropa[c]quinolin-3(7bH)-yl)pyrrolidine-1-carboxylate (2.5 g, 7.17 mmol) in acetic acid (40 mL) was added iron(III) chloride (581 mg, 3.58 mmol) and 1,3-dibromo-5,5-dimethylimidazolidine-2,4-dione (1.02 g, 3.58 mmol). After stirred at 20° C. for 1 h, the mixture was concentrated. Then the residue was diluted with water (60 mL) and extracted with ethyl acetate (40 mL×2). The combined organic layers were washed with brine (50 mL), dried over anhydrous sodium sulfate and concentrated under reduced pressure. The residue was purified by column chromatography to afford the title compound. LCMS R_{T}=1.917 min, m/z=429.1 [M+H+2].

### Step 3

To a solution of (2-(((tert-butyldimethylsilyl)oxy)methyl)thieno[3,2-b]pyridin-7-yl)boronic acid (0.9 g, 2.78 mmol), tert-butyl 3-(4-bromo-6-chloro-1a,2-dihydro-1H-cyclopropa[c]quinolin-3(7bH)-yl)pyrrolidine-1-carboxylate (1.19 g, 2.78 mmol) in dioxane (40 mL) and water (2 mL) was added potassium carbonate (962 mg, 6.96 mmol) and dichloro[1,1′-bis(diphenylphosphino)ferrocene]palladium(II) (407 mg, 0.56 mmol). After stirred at 100° C. for 2 h under nitrogen atmosphere, the mixture was filtered and the filtrate was concentrated under reduced pressure. The residue was purified by column chromatography. The racemate was separated by SFC to first eluting fraction (113.1-i, Rt=4.070 min, LCMS R_{T}=1.743 min, m/z=626.5 [M+H]+), second eluting fraction (113.1-ii, Rt=4.488 min, LCMS R_{T}=1.743 min, m/z=626.5 [M+H]+), third eluting fraction (113.1-iii, Rt=4.811 min, LCMS R_{T}=1.743 min, m/z=626.5 [M+H]+) and fourth eluting fraction (113.1-iv, Rt=5.433 min, LCMS R_{T}=1.743 min, m/z=626.5 [M+H]+). LCMS R_{T}=1.747 min, m/z=626.5 [M+H]⁺.

### Step 4: tert-butyl 3-(6-chloro-4-(2-(hydroxymethyl)thieno[3,2-b]pyridin-7-yl)-1,1a,2,7b-tetrahydro-3H-cyclopropa[c]quinolin-3-yl)pyrrolidine-1-carboxylate

The diastereomers from Step 4 (70 mg, 0.11 mmol) in THF (1 mL) was treated with tetrabutylammonium fluoride (1 M in THF, 0.11 mL). After stirred at 25° C. for 0.5 h, the mixture was purified by preparative TLC to afford 113.2-i, 113.2-ii, 113.2-iii and 113.2-iv. LCMS R_{T}=1.878 min, m/z=512.1 [M+H]⁺.

### Step 5

Example 113a was prepared from 113.2-i and succinimide, following the procedure described in the synthesis of Example 78. The product was purified by preparative HPLC. ¹H NMR (400 MHz, CD₃OD) δ=8.75 (d, J=4.8 Hz, 1H), 8.491 (s, 1H), 7.51-7.39 (m, 2H), 7.35 (d, J=2.5 Hz, 1H), 7.03 (s, 1H), 4.95 (s, 2H), 3.75 (s, 1H), 3.25-3.08 (m, 2H), 2.76 (s, 6H), 2.29-1.72 (m, 5H), 1.36-1.07 (m, 2H), 0.89 (J=3.6 Hz, 1H). LCMS R_{T}=1.348 min, m/z=493.2 [M+H]⁺.

Example 113b was prepared from 113.2-ii and succinimide, following the procedure described in the synthesis of Example 78. ¹H NMR (400 MHz, CD₃OD) δ=8.69 (d, J=4.8 Hz, 1H), 8.52 (s, 1H), 7.52-7.31 (m, 3H), 7.04 (s, 1H), 4.94 (s, 2H), 3.66 (s, 1H), 3.39 (dd, J=2.4, 13.8 Hz, 1H), 3.18 (d, J=14.0 Hz, 1H), 3.05-2.54 (m, 9H), 2.11-1.86 (m, 2H), 1.40-1.04 (m, 2H), 0.76 (s, 1H). LCMS R_{T}=1.007 min, m/z=493.2 [M+H]⁺.

Example 113c was prepared from 113.2-iii and succinimide, following the procedure described in the synthesis of Example 78. ¹H NMR (400 MHz, CD₃OD) δ=8.69 (d, J=4.8 Hz, 1H), 8.51 (s, 1H), 7.52-7.31 (m, 3H), 7.04 (s, 1H), 4.94 (s, 2H), 3.66 (s, 1H), 3.39 (dd, J=2.4, 13.2 Hz, 1H), 3.18 (d, J=13.2 Hz, 1H), 3.12-2.26 (m, 8H), 2.13-1.90 (m, 2H), 1.36-1.02 (m, 2H), 1.29-1.02 (m, 1H), 0.76 (s, 1H). LCMS R_{T}=1.312 min, m/z=493.2 [M+H]⁺.

Example 113d was prepared from 113.2-iv and succinimide, following the procedure described in the synthesis of Example 78. ¹H NMR (400 MHz, CD₃OD) δ=8.72 (d, J=4.8 Hz, 1H), 8.52 (s, 1H), 7.51-7.39 (m, 2H), 7.35 (d, J=2.5 Hz, 1H), 7.03 (s, 1H), 4.95 (s, 2H), 3.75 (s, 1H), 3.25-3.08 (m, 2H), 2.76 (s, 6H), 2.29-1.72 (m, 5H), 1.36-1.07 (m, 2H), 0.89 (J=3.6 Hz, 1H). LCMS R_{T}=1.368 min, m/z=493.2 [M+H]⁺.

The absolute configuration was tentatively assigned based on the relative potency of the pair diastereomers in biology assays.

### Example 114: 3-((7-((1aS,7bR)-6-chloro-3-((S)-pyrrolidin-3-yl)-1a,2,3,7b-tetrahydro-1H-cyclopropa[c]quinolin-4-yl)thieno[3,2-b]pyridin-2-yl)methyl)-1-(2,2,2-trifluoroethyl)pyrimidine-2,4(1H,3H)-dione

To a solution of tert-butyl (3S)-3-[(laS,7bR)-6-chloro-4-[2-(hydroxymethyl)thieno[3,2-b]pyridin-7-yl]-1,1a,2,7b-tetrahydrocyclopropa[c]quinolin-3-yl]pyrrolidine-1-carboxylate (113.2-iv, 40 mg, 0.078 mmol) and 1-(2,2,2-trifluoroethyl)pyrimidine-2,4-dione (30 mg, 0.16 mmol) in THF (1 mL) was added diisopropylazodicarboxylate (47 mg, 0.23 mmol) and triphenylphosphine (61 mg, 0.23 mmol). After the mixture was stirred at 50° C. for 1 h, the reaction mixture was concentrated under reduced pressure to remove the solvent. The residue was dissolved in dioxane (5 mL) and added hydrochloric acid (6 mL, 4 M in dioxane). After stirred at 25° C. for 30 min, the reaction mixture was concentrated under reduced pressure. The residue was purified by preparative HPLC to afford the title compound. ¹H NMR (400 MHz, CD₃OD) δ=8.66 (d, J=4.8 Hz, 1H), 7.63 (d, J=8.0 Hz, 1H), 7.52 (s, 1H), 7.43-7.28 (m, 2H), 6.98 (s, 1H), 5.87 (d, J=8.0 Hz, 1H), 5.44-5.30 (m, 2H), 4.68-4.55 (m, 2H), 3.50 (d, J=16.0 Hz, 1H), 3.30-3.08 (m, 3H), 2.75 (s, 2H), 2.33 (d, J=6.8 Hz, 1H), 2.05-1.89 (m, 2H), 1.59 (s, 2H), 1.07 (8.0 Hz, 1H), 0.92 (s, 1H). LCMS R_{T}=1.533 min, m/z=588.3 [M+H]⁺.

### Example 115: 3-[[7-[(1aS,3R,7bR)-6-chloro-3-pyrrolidin-3-yl-1,1a,2,7b-tetrahydrocyclopropa[c]quinolin-4-yl]thieno[3,2-b]pyridin-2-yl]methyl]-1-(2,2-difluoroethyl)pyrimidine-2,4-dione

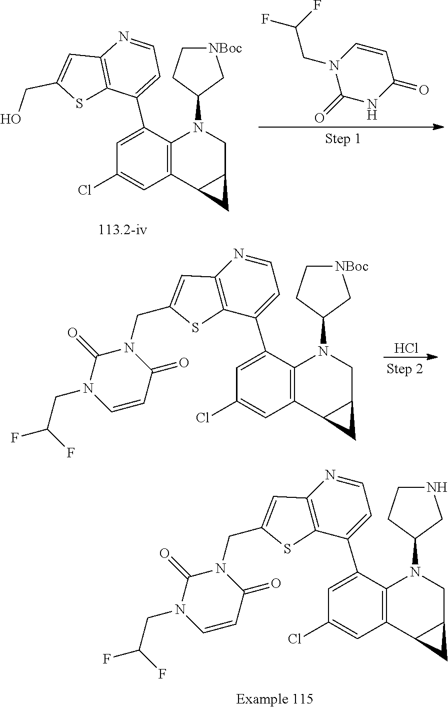

### Step 1: (R)-tert-butyl 3-((1aS,7bR)-6-chloro-4-(2-((3-(2,2-difluoroethyl)-2,6-dioxo-2,3-dihydropyrimidin-1(6H)-yl)methyl)thieno[3,2-b]pyridin-7-yl)-1a,2-dihydro-1H-cyclopropa[c]quinolin-3(7bH)-yl)pyrrolidine-1-carboxylate

To a solution of (S)-tert-butyl 3-((1aS,7bR)-6-chloro-4-(2-(hydroxymethyl)thieno[3,2-b]pyridin-7-yl)-1a,2-dihydro-1H-cyclopropa[c]quinolin-3(7bH)-yl)pyrrolidine-1-carboxylate (40 mg, 0.078 mmol) and 1-(2,2-difluoroethyl)pyrimidine-2,4-dione (28 mg, 0.16 mmol) in THF (1 mL) was added diisopropylazodicarboxylate (47 mg, 0.23 mmol) and triphenylphosphine (61 mg, 0.23 mmol). After the mixture was stirred at 50° C. for 1 h, the reaction mixture was concentrated under reduced pressure. The residue was purified by column chromatography to afford the title compound. LCMS R_{T}=0.677 min, m/z=670.4 [M+H]⁺.

### Step 2: 3-[[7-[(1aS,3R,7bR)-6-chloro-3-pyrrolidin-3-yl-1,1a,2,7b-tetrahydrocyclopropa[c]quinolin-4-yl]thieno[3,2-b]pyridin-2-yl]methyl]-1-(2,2-difluoroethyl)pyrimidine-2,4-dione

To a solution of (R)-tert-butyl 3-((1aS,7bR)-6-chloro-4-(2-((3-(2,2-difluoroethyl)-2,6-dioxo-2,3-dihydropyrimidin-1(6H)-yl)methyl)thieno[3,2-b]pyridin-7-yl)-1a,2-dihydro-1H-cyclopropa[c]quinolin-3(7bH)-yl)pyrrolidine-1-carboxylate (120 mg, 018 mol) in dioxane (2 ml) was added dioxane hydrochloride (5 mL). After the mixture was stirred at 25° C. for 0.5 h, the reaction mixture was concentrated under reduced pressure. The residue was purified by preparative HPLC to afford the title compound. ¹H NMR (400 MHz, CD₃OD) δ=8.66 (d, J=4.8 Hz, 1H), 7.59 (d, J=8.0 Hz, 1H), 7.52 (s, 1H), 7.37 (s, 1H), 7.29 (d, J=2.4 Hz, 1H), 6.98 (s, 1H), 6.29-6.01 (m, 1H), 5.82 (d, J=8.0 Hz, 1H), 5.37 (s, 2H), 4.21 (d, J=3.6, 14.4 Hz, 2H), 3.59-3.45 (m, 1H), 3.30-3.12 (m, 3H), 2.76 (s, 2H), 2.40-2.22 (m, 1H), 2.08-1.90 (m, 2H), 1.58 (d, J=8.4 Hz, 2H), 1.07 (d, J=4.8, 8.2 Hz, 1H), 0.92 (s, 1H). LCMS R_{T}=1.447 min, m/z=570.3 [M+H]⁺.

### Example 116: (S)-1-((7-(6-chloro-1-(pyrrolidin-3-yl)-1,2,3,4-tetrahydroquinolin-8-yl)-5-methylthieno[3,2-b]pyridin-2-yl)methyl)pyrrolidine-2,5-dione, formic acid salt

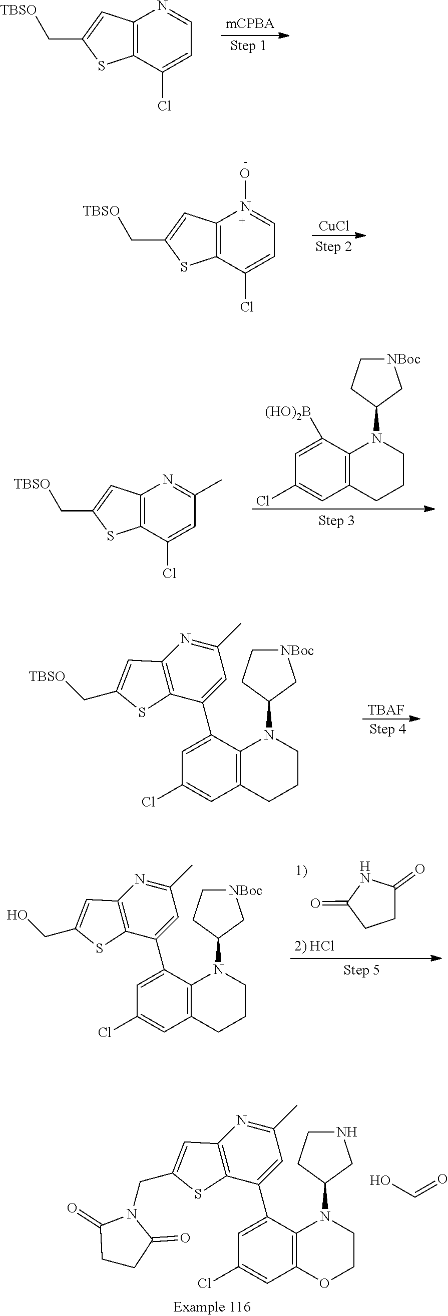

### Step 1: 2-(((tert-butyldimethylsilyl)oxy)methyl)-7-chlorothieno[3,2-b]pyridine 4-oxide

To a solution of 2-(((tert-butyldimethylsilyl)oxy)methyl)-7-chlorothieno[3,2-b]pyridine (10.0 g, 31.86 mmol) in dichloromethane (50 mL) was added 3-chloroperbenzoic acid (9.7 g, 47.78 mmol). After stirred at 0° C. for 16 h, the reaction mixture was diluted with sodium carbonate (100 mL) and extracted with dichloromethane (50 mL×2). The combined organic layers were washed with sodium thiosulfate (250 mL), dried over anhydrous sodium sulfate and concentrated under reduced pressure to give a residue. The residue was purified by column chromatography to afford the title compound. LCMS R_{T}=0.612 min, m/z=330.2 [M+H]⁺.

### Step 2: 2-(((tert-butyldimethylsilyl)oxy)methyl)-7-chloro-5-methylthieno[3,2-b]pyridine

To a mixture of 2-(((tert-butyldimethylsilyl)oxy)methyl)-7-chlorothieno[3,2-b]pyridine 4-oxide (5.0 g, 15.16 mmol), copper chloride (150 mg, 1.52 mmol) and magnesium chloride (3.0 g, 30.31 mmol) in THF (30 mL) was added bromo(methyl)magnesium (3 M in THF, 30.31 mmol) at 0° C. It was allowed to warm to 25° C. and stirred for 16 h. The reaction mixture was quenched by addition of water (20 mL), extracted by ethyl acetate (20 mL×3). The combined organic layers were washed with brine (20 mL), dried over anhydrous sodium sulfate and concentrated to give a residue. The residue was purified by preparative HPLC to afford the title compound. LCMS R_{T}=0.787 min, m/z=328.0 [M+H]⁺.

### Step 3: (S)-tert-butyl 3-(8-(2-(((tert-butyldimethylsilyl)oxy)methyl)-5-methylthieno[3,2-b]pyridin-7-yl)-6-chloro-3,4-dihydroquinolin-1(2H)-yl)pyrrolidine-1-carboxylate

To a solution of 2-(((tert-butyldimethylsilyl)oxy)methyl)-7-chloro-5-methylthieno[3,2-b]pyridine (580 mg, 1.52 mmol) in dioxane (1 mL) and water (1 mL) was added tert-butyl-[(7-chloro-5-methyl-thieno[3,2-b]pyridin-2-yl)methoxy]-dimethyl-silane (500 mg, 1.52 mmol), cesium carbonate (994 mg, 3.05 mmol) and dichloro[1,1′-bis(diphenylphosphino)ferrocene]palladium(II) (223 mg, 0.31 mmol). After stirred at 110° C. for 3 h, the reaction mixture was diluted with water (15 mL) and extracted with ethyl acetate (30 mL×3). The combined organic layers were washed with brine (50 mL), dried over anhydrous sodium sulfate, filtered and the filtrate was concentrated under reduced pressure. The residue was purified by column chromatography to afford the title compound. LCMS R_{T}=1.015, m/z=628.5 [M+H]⁺.

### Step 4: (S)-tert-butyl 3-(6-chloro-8-(2-(hydroxymethyl)-5-methylthieno[3,2-b]pyridin-7-yl)-3,4-dihydroquinolin-1(2H)-yl)pyrrolidine-1-carboxylate

To a solution of (S)-tert-butyl 3-(8-(2-(((tert-butyldimethylsilyl)oxy)methyl)-5-methylthieno[3,2-b]pyridin-7-yl)-6-chloro-3,4-dihydroquinolin-1(2H)-yl)pyrrolidine-1-carboxylate (300 mg, 0.48 mmol) in THF (10 mL) was added tetrabutylammonium fluoride (10 mL, 1.0 M in THF) and stirred at 25° C. for 0.5 h. The reaction mixture was concentrated under reduced pressure. The residue was purified by column chromatography to afford the title compound. LCMS R_{T}=0.808, m/z=514.3 [M+H]⁺.

### Step 5

To a solution of (S)-tert-butyl 3-(6-chloro-8-(2-(hydroxymethyl)-5-methylthieno[3,2-b]pyridin-7-yl)-3,4-dihydroquinolin-1(2H)-yl)pyrrolidine-1-carboxylate (50 mg, 0.10 mmol), succinimide (20 mg, 0.20 mmol) and tetramethylazodicarboxamide (50 mg, 0.30 mmol) in toluene (2 mL) was added tributylphosphane (118 mg, 0.58 mmol) at 25° C. After stirred at 95° C. for 1 h, the reaction mixture was concentrated under reduced pressure. The residue was dissolved in hydrochloric acid (2 mL, 4 M in dioxane). After stirred at 20° C. for 30 min, the reaction mixture was concentrated under reduced pressure. The residue was purified by preparative HPLC to afford Example 116. ¹H NMR (400 MHz, CD₃OD) δ=7.38 (s, 2H), 7.17-7.12 (m, 2H), 4.92-4.91 (m, 2H), 3.70 (m, J=8.4 Hz, 1H), 3.29-3.10 (m, 4H), 2.88 (t, J=6.4 Hz, 3H), 2.75 (s, 6H), 2.71 (s, 3H), 1.94 (m, J=6.4 Hz, 3H). LCMS R_{T}=1.210, m/z=495.4 [M+H]⁺.

### Example 117a: (1aR,7bS)-4-(2-((3-(2,2-difluoroethyl)-2,6-dioxo-3,6-dihydropyrimidin-1(2H)-yl)methyl)thieno[3,2-b]pyridin-7-yl)-3-((R)-pyrrolidin-3-yl)-1a,2,3,7b-tetrahydro-1H-cyclopropa[c]quinoline-6-carbonitrile, formic acid salt

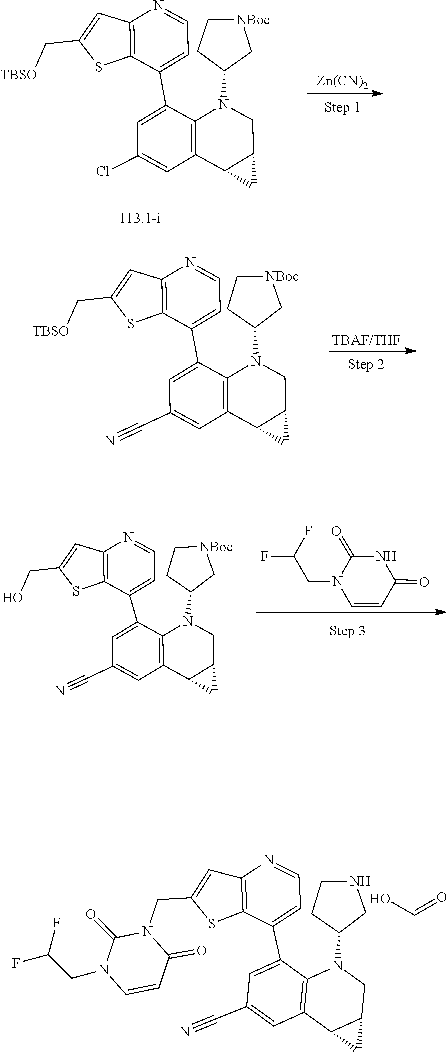

### Step 1: (R)-tert-butyl 3-((1aR,7bS)-4-(2-(((tert-butyldimethylsilyl)oxy)methyl)thieno[3,2-b]pyridin-7-yl)-6-cyano-1a,2-dihydro-1H-cyclopropa[c]quinolin-3(7bH)-yl)pyrrolidine-1-carboxylate

To a solution of 113.1-i (160 mg, 0.26 mmol) in dioxane (3 mL) was added zinc cyanide (39 mg, 0.332 mmol), diisopropylethylamine (82 mg, 0.64 mmol) and palladium; tritert-butylphosphane (26 mg, 0.05 mmol). After the mixture was stirred at 90° C. for 15 h, the reaction mixture was concentrated under reduced pressure. The residue was purified by preparative TLC to afford the title compound. LCMS R_{T}=1.053 min, m/z=617.2 [M+H]⁺

### Step 2: (R)-tert-butyl 3-((1aR,7bS)-6-cyano-4-(2-(hydroxymethyl)thieno[3,2-b]pyridin-7-yl)-1a,2-dihydro-1H-cyclopropa[c]quinolin-3(7bH)-yl)pyrrolidine-1-carboxylate

To a solution of (R)-tert-butyl 3-((1aR,7bS)-4-(2-(((tert-butyldimethylsilyl)oxy)methyl)thieno[3,2-b]pyridin-7-yl)-6-cyano-1a,2-dihydro-1H-cyclopropa[c]quinolin-3(7bH)-yl)pyrrolidine-1-carboxylate (90 mg, 0.15 mmol) in THF (3 mL) was added tetrabutylfluramide (1 M, 0.15 mL). After the mixture was stirred at 20° C. for 1 h, the reaction mixture was concentrated under reduced pressure. The residue was purified by preparative TLC to afford the title compound. LCMS R_{T}=0.591 min, m/z=503.3 [M+H]⁺.

### Step 3

Example 117a was prepared from (R)-tert-butyl 3-((1aR,7bS)-6-cyano-4-(2-(hydroxymethyl)thieno[3,2-b]pyridin-7-yl)-1a,2-dihydro-1H-cyclopropa[c]quinolin-3(7bH)-yl)pyrrolidine-1-carboxylate and 1-(2,2-difluoroethyl)pyrimidine-2,4-dione (35 mg, 0.2 mmol), following the procedure described in the synthesis of Example 78. The product was purified by preparative HPLC. ¹H NMR (400 MHz, CD₃OD) δ=8.76 (d, J=3.6 Hz, 1H), 8.53 (s, 1H), 7.65 (d, J=2.0 Hz, 1H), 7.61 (d, J=8.0 Hz, 1H), 7.55 (s, 1H), 7.51 (d, J=4.0 Hz, 1H), 7.37 (s, 1H), 6.29 (t, J=3.6 Hz, 1H), 5.83 (d, J=8.0 Hz, 1H), 5.38 (s, 2H), 4.32-4.13 (m, 2H), 3.94-3.76 (m, 1H), 3.29-3.17 (m, 1H), 3.15-2.96 (m, 1H), 2.22-1.97 (m, 5H), 1.80 (d, J=4.8 Hz, 2H), 1.22-0.84 (m, 3H). LCMS: m/z=561.1 [M+H]⁺.

### Example 117b: (1aS,7bR)-4-(2-((3-(2,2-difluoroethyl)-2,6-dioxo-2,3-dihydropyrimidin-1(6H)-yl)methyl)thieno[3,2-b]pyridin-7-yl)-3-((R)-pyrrolidin-3-yl)-1a,2,3,7b-tetrahydro-1H-cyclopropa[c]quinoline-6-carbonitrile

Example 117b was prepared from 113.1-ii following the procedures described in the synthesis of Example 117a. The final product was purified by preparative HPLC. ¹H NMR (400 MHz, CD₃OD) δ=8.70 (d, J=4.0 Hz, 1H), 7.66-7.11 (m, 5H), 6.33-5.96 (m, 1H), 5.82 (d, J=8.0 Hz, 1H), 5.37 (s, 2H), 4.21 (dt, J=3.6, 14.4 Hz, 2H), 3.60 (d, J=8.0 Hz, 1H), 3.41-3.32 (m, 1H), 3.24-3.11 (m, 1H), 2.88-2.28 (m, 5H), 2.14-1.96 (m, 2H), 1.15-0.68 (m, 3H). LCMS: m/z=561.3 [M+H]⁺.

### Example 117c: (1aR,7bS)-4-(2-((3-(2,2-difluoroethyl)-2,6-dioxo-3,6-dihydropyrimidin-1(2H)-yl)methyl)thieno[3,2-b]pyridin-7-yl)-3-((S)-pyrrolidin-3-yl)-1a,2,3,7b-tetrahydro-1H-cyclopropa[c]quinoline-6-carbonitrile

Example 117c was prepared from 113.1-iii following the procedures described in the synthesis of Example 117a. The final product was purified by preparative HPLC. ¹H NMR (400 MHz, CD₃OD) δ=8.73 (s, 1H), 8.53 (s, 1H), 7.33-7.71 (m, 5H), 6.25-6.00 (m, 1H), 5.82 (d, J=8.0 Hz, 1H), 5.37 (s, 2H), 4.13-4.32 (m, 2H), 3.93-3.73 (m, 1H), 3.59-3.33 (m, 1H), 3.25-2.75 (m, 1H), 2.56-3.16 (m, 6H), 2.22-1.96 (m, 2H), 0. 1.18-81 (m, 2H). LCMS: m/z=561.3 [M+H]⁺

### Example 117d: (1aS,7bR)-4-[2-[[3-(2,2-difluoroethyl)-2,6-dioxo-pyrimidin-1-yl]methyl]thieno[3,2-b]pyridin-7-yl]-3-[(3S)-pyrrolidin-3-yl]-1,1a,2,7b-tetrahydrocyclopropa[c]quinoline-6-carbonitrile, formic acid salt

Example 117d was prepared from 113-1-iv following the procedures described in the synthesis of Example 117a. The final product was purified by preparative HPLC. ¹H NMR (400 MHz, CD₃OD) δ=8.76 (d, J=4.0 Hz, 1H), 8.51 (s, 1H), 7.66 (d, J=2.0 Hz, 1H), 7.61 (d, J=8.0 Hz, 1H), 7.56-7.49 (m, 2H), 7.37 (s, 1H), 6.28 (t, J=3.6 Hz, 1H), 5.82 (d, J=8.0 Hz, 1H), 5.37 (s, 2H), 4.28-4.17 (m, 2H), 3.95-3.86 (m, 1H), 3.37-3.33 (m, 1H), 3.27-3.13 (m, 1H), 2.85-2.02 (m, 5H), 1.86 (d, J=4.0 Hz, 2H), 0.96 (d, J=4.0 Hz, 3H). LCMS: m/z=561.3 [M+H]⁺.

### Example 118a and 118b: (R)-3-((7-(6-chloro-1-(5,5-dimethylpyrrolidin-3-yl)-1,2,3,4-tetrahydroquinolin-8-yl)thieno[3,2-b]pyridin-2-yl)methyl)-1-(2,2,2-trifluoroethyl)pyrimidine-2,4(1H,3H)-dione, formic acid salt and (S)-3-((7-(6-chloro-1-(5,5-dimethylpyrrolidin-3-yl)-1,2,3,4-tetrahydroquinolin-8-yl)thieno[3,2-b]pyridin-2-yl)methyl)-1-(2,2,2-trifluoroethyl)pyrimidine-2,4(1H,3H)-dione, formic acid salt

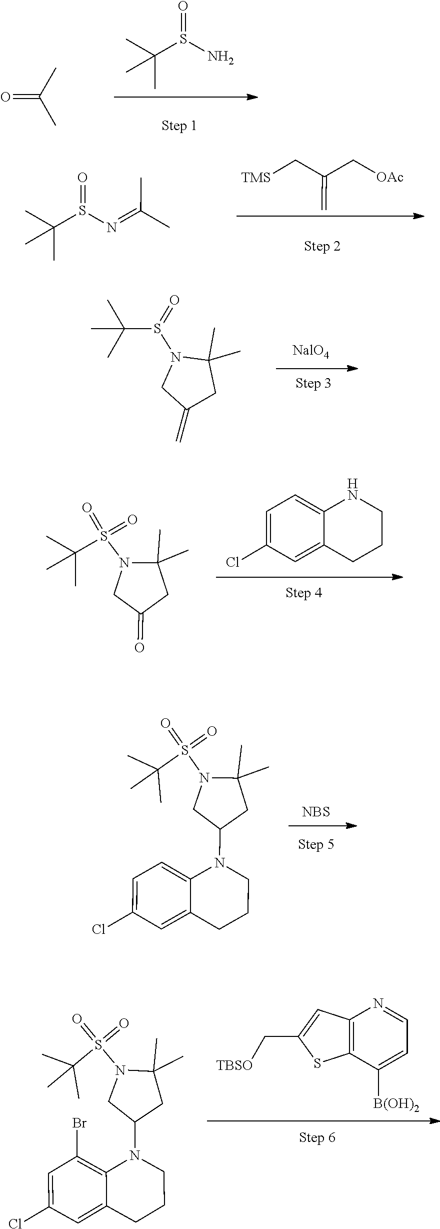

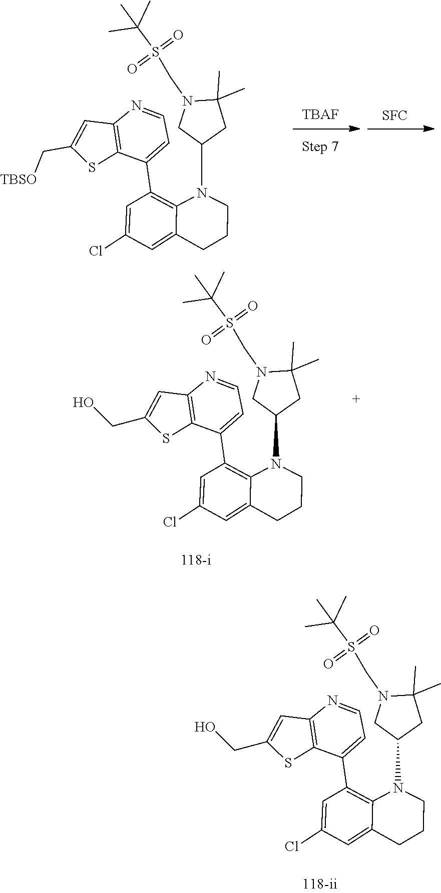

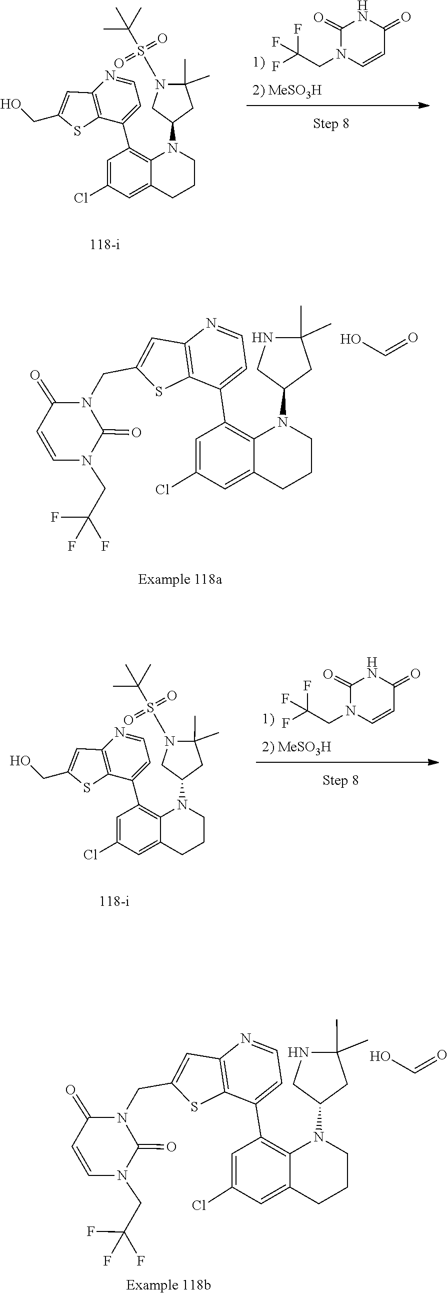

### Step 1: 2-methyl-N-(propan-2-ylidene) propane-2-sulfinamide

To a solution of propan-2-one (10.0 g, 172.18 mmol) and 2-methylpropane-2-sulfinamide (18.8 g, 154.96 mmol) in THF (340 mL) was slowly added titanium (IV) isopropoxide (73.4 g, 258.27 mmol). After stirred at 20° C. for 16 h, the reaction solution was slowly poured into ice-water (100 mL). The suspension was filtered through celite, and the filter cake was washed with ethyl acetate (100 mL×2). The separated organic layers were dried over sodium sulfate, concentrated. The residue was purified by column chromatography to afford the title compound. ¹H NMR (400 MHz, CDCl₃) δ=2.34 (s, 3H), 2.19 (s, 3H), 1.23 (s, 9H).

### Step 2: 1-(tert-butylsulfinyl)-2, 2-dimethyl-4-methylenepyrrolidine

To a solution of 2-methyl-N-(propan-2-ylidene)propane-2-sulfinamide (5.5 g, 34.11 mmol) and 2-(trimethylsilylmethyl)allyl acetate (9.5 g, 51.16 mmol) in THF (150 mL) was added tetrakis[triphenylphosphine]palladium (3.9 g, 3.41 mmol) under nitrogen atmosphere. After stirred at 20° C. for 48 h under nitrogen atmosphere, the reaction mixture was filtered and the filtrate was concentrated. The residue was purified by column chromatography to afford the title compound. ¹H NMR (400 MHz, CDCl₃) δ=4.97 (br, 2H), 4.26-4.18 (m, 1H), 3.53 (br, 1H), 2.49-2.26 (m, 2H), 1.30 (s, 6H), 1.22 (s, 9H).

### Step 3: 1-(tert-butylsulfonyl)-5, 5-dimethylpyrrolidin-3-one

To a solution of 1-tert-butylsulfinyl-2, 2-dimethyl-4-methylenepyrrolidine (4.8 g, 22.29 mmol) in acetonitrile (80 mL) and water (80 mL) was added sodium periodate (19.1 g, 89.16 mmol) and ruthenium (III) chloride (231 mg, 1.11 mmol). After stirred at 20° C. for 16 h, the reaction mixture was partitioned between ethyl acetate (100 mL) and water (100 mL). The organic phase was separated. The aqueous phase was extracted with ethyl acetate (80 mL). The combined organic layer was dried over sodium sulfate, filtered and the filtrate was concentrated under reduced pressure. The residue was purified by column chromatography to afford the title compound. ¹H NMR (400 MHz, CD₃OD) δ=3.91 (s, 2H), 2.51 (s, 2H), 1.64 (s, 6H), 1.44 (s, 9H).

### Step 4: 1-(1-(tert-butylsulfonyl)-5, 5-dimethylpyrrolidin-3-yl)-6-chloro-1, 2, 3, 4-tetrahydroquinoline

To a solution of 6-chloro-1,2,3,4-tetrahydroquinoline (500 mg, 2.98 mmol) and 1-(tert-butylsulfonyl)-5, 5-dimethylpyrrolidin-3-one (835 mg, 3.58 mmol) in acetic acid (5 mL) was added sodium triacetyloxyboranuide (1.9 g, 8.95 mmol). After stirred at 20° C. for 2 h, the reaction mixture was quenched by addition of water (20 ml) and extracted with ethyl acetate (20 mL×2). The combined organic layers were washed with brine (30 mL), dried over anhydrous sodium sulfate, filtered and the filtrate was concentrated under reduced pressure. The residue was purified by column chromatography to afford the title compound. LCMS R_{T}=1.068 min, m/z=385.0 [M+H]⁺

### Step 5: 8-bromo-1-(1-(tert-butylsulfonyl)-5,5-dimethylpyrrolidin-3-yl)-6-chloro-1,2,3,4-tetrahydroquinoline

To a solution of 1-(1-(tert-butylsulfonyl)-5, 5-dimethylpyrrolidin-3-yl)-6-chloro-1, 2, 3, 4-tetrahydroquinoline (190 mg, 493.55 mmol) in DMF (4 mL) was added 1-bromopyrrolidine-2,5-dione (88 mg, 0.48 mmol). After stirred at 0° C. for 1 h. The reaction mixture was quenched by addition of water (20 ml) and extracted with ethyl acetate (20 mL×2). The combined organic layers were washed with brine (20 mL), dried over anhydrous sodium sulfate, filtered and the filtrate was concentrated under reduced pressure. The residue was purified by column chromatography to afford the title compound. LCMS R_{T}=0.667 min, m/z=465.1 [M+2+H]⁺.

### Step 6: 2-(((tert-butyldimethylsilyl)oxy)methyl)-7-(1-(1-(tert-butylsulfonyl)-5,5-dimethylpyrrolidin-3-yl)-6-chloro-1,2,3,4-tetrahydroquinolin-8-yl)thieno[3,2-b]pyridine

A mixture of (2-(((tert-butyldimethylsilyl)oxy)methyl)thieno[3,2-b]pyridin-7-yl)boronic acid (309 mg, 0.95 mmol), 8-bromo-1-(1-(tert-butylsulfonyl)-5,5-dimethylpyrrolidin-3-yl)-6-chloro-1,2,3,4-tetrahydroquinoline (355 mg, 0.76 mmol), cyclopenta-2,4-dien-1-yl(diphenyl)phosphane dichloropalladium iron(II) (70 mg, 0.95 mmol) and potassium carbonate (264 mg, 1.91 mmol) in dioxane (15 mL) and water (3 mL) was degassed and purged with nitrogen for 3 times. The mixture was stirred at 100° C. for 3 h under nitrogen atmosphere. It was concentrated. The residue was purified by column chromatography to afford the title compound. LCMS R_{T}=0.668 min, m/z=662.1 [M+H]⁺.

### Step 7: (R)-(7-(1-(1-(tert-butylsulfonyl)-5,5-dimethylpyrrolidin-3-yl)-6-chloro-1,2,3,4-tetrahydroquinolin-8-yl)thieno[3,2-b]pyridin-2-yl)methanol and (S)-(7-(1-(1-(tert-butylsulfonyl)-5,5-dimethylpyrrolidin-3-yl)-6-chloro-1,2,3,4-tetrahydroquinolin-8-yl)thieno[3,2-b]pyridin-2-yl)methanol

To a solution of 2-(((tert-butyldimethylsilyl)oxy)methyl)-7-(1-(1-(tert-butylsulfonyl)-5,5-dimethylpyrrolidin-3-yl)-6-chloro-1,2,3,4-tetrahydroquinolin-8-yl)thieno[3,2-b]pyridine (500 mg, 0.75 mmol) in THF (5 mL) was added tetrabutylammonium fluoride (0.8 mL, 1.0 M in THF). After stirred at 20° C. for 1 h. The reaction mixture was concentrated. The residue was purified by column chromatography. The racemate (500 mg, 0.75 mmol) was separated by SFC to afford first eluting fraction (118-i, Rt=2.661 min; LCMS R_{T}=0.523 min, m/z=548.3 [M+H]+) and second eluting fraction (118-ii, Rt=3.567 min; LCMS R_{T}=0.527 min, m/z=548.3 [M+H]+).

### Step 8

To a solution of 118-i (80 mg, 0.15 mmol), 1-(2,2,2-trifluoroethyl)pyrimidine-2,4(1H,3H)-dione (28 mg, 0.15 mmol) and triphenylphosphane (115 mg, 0.44 mmol) in THF (1 mL) was added diisopropyl azodicarboxylate (88 mg, 0.44 mmol) at 25° C. After stirred at 50° C. for 2 h, the reaction mixture was concentrated. The residue was dissolved in trifluoromethanesulfonic acid (82 mg, 0.55 mmol). After stirred at 20° C. for 1 h, the reaction mixture was concentrated under reduced pressure. The residue was purified by preparative HPLC to afford Example 118a. ¹H NMR (400 MHz, CD₃OD) δ=8.73 (d, J=4.8 Hz, 1H), 8.54 (s, 1H), 7.74-7.39 (m, 3H), 7.24-7.10 (m, 2H), 5.89 (d, J=7.6 Hz, 1H), 5.41 (s, 2H), 4.69-4.65 (m, 2H), 3.78 (br s, 1H), 3.29-3.10 (m, 2H), 3.06-2.42 (m, 3H), 1.92 (br s, 3H), 1.61-1.09 (m, 4H), 0.72 (br s, 4H). LCMS R_{T}=1.396 min, m/z=604.3 [M+H]⁺.

Example 118b was prepared from 118-ii following the procedure described in the synthesis of Example 118a. The absolute configuration was tentatively assigned based on the relative potency of the pair diastereomers in biology assays.

### Example 119: (S)-1-((7-(6-chloro-1-(5,5-dimethylpyrrolidin-3-yl)-1,2,3,4-tetrahydroquinolin-8-yl)thieno[3,2-b]pyridin-2-yl)methyl)pyrrolidine-2,5-dione, formic acid salt

Prepared from 118.ii (60 mg, 0.11 mmol) and succinimide (43 mg, 0.44 mmol), following the procedure described in the synthesis of Example 108a. The residue was purified by preparative HPLC. ¹HNMR (400 MHz, CD₃OD) δ=8.75-8.71 (m, 1H), 8.50 (s, 1H), 7.52-7.42 (m, 2H), 7.21-7.13 (m, 2H), 4.97-4.91 (m, 2H), 3.97-3.64 (m, 2H), 3.27-3.04 (m, 3H), 3.03-2.86 (m, 3H), 2.77-2.75 (m, 4H), 1.85 (s, 3H), 1.31-1.23 (m, 3H), 0.78-0.68 (m, 3H). LCMS R_{T}=1.270 min, m/z=509.3 [M+H]+

### Example 120a and 120b: (S)-1-(5,5-dimethylpyrrolidin-3-yl)-8-(2-((4-methyl-6-oxopyridazin-1(6H)-yl)methyl)thieno[3,2-b]pyridin-7-yl)-1,2,3,4-tetrahydroquinoline-6-carbonitrile, formic acid salt and (R)-1-(5,5-dimethylpyrrolidin-3-yl)-8-(2-((4-methyl-6-oxopyridazin-1(6H)-yl)methyl)thieno[3,2-b]pyridin-7-yl)-1,2,3,4-tetrahydroquinoline-6-carbonitrile, formic acid salt

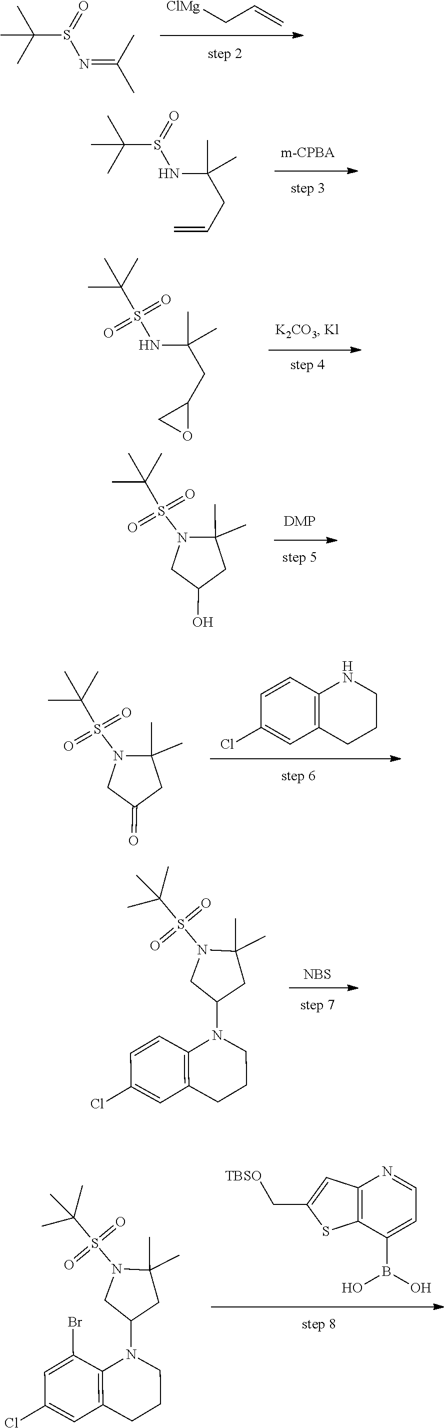

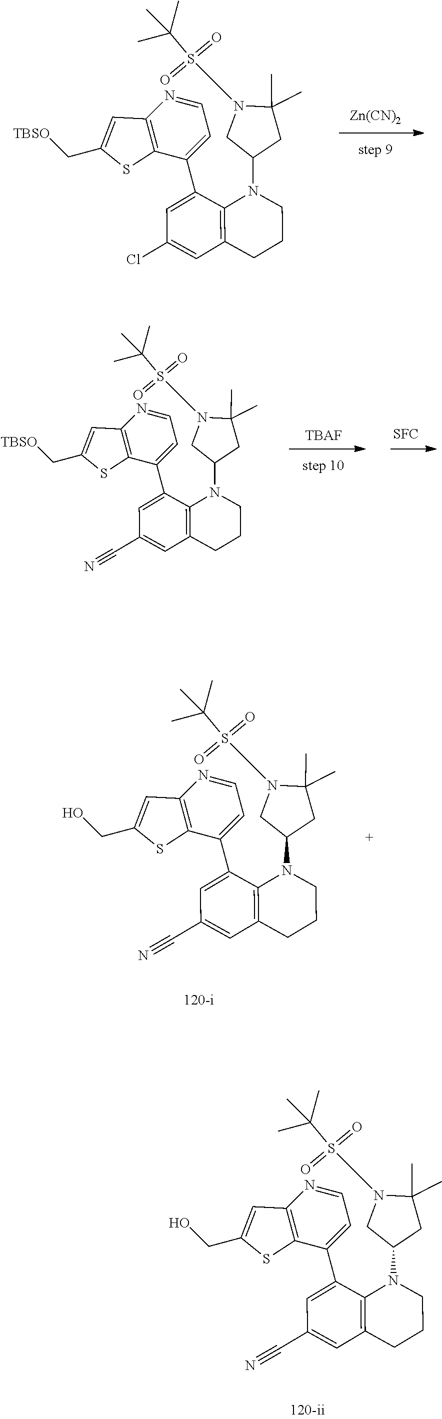

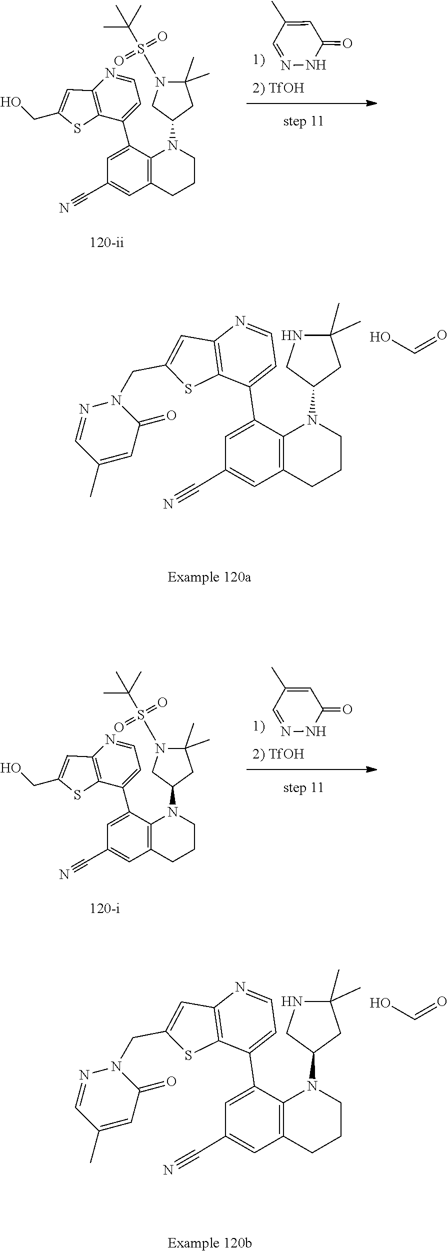

### Step 1: 2-methyl-N-(propan-2-ylidene)propane-2-sulfinamide

To a solution of 2-methylpropane-2-sulfinamide (58.4 g, 482.10 mmol) in DMF (300 mL) was added acetone (40 g, 688.72 mmol) and tetraisopropoxytitanium (293.6 g, 1.03 mol) at 20° C. After stirred at 20° C. for 16 h, the reaction mixture was quenched by addition of water (400 mL) and extracted with ethyl acetate (600 mL). The combined organic layers were washed with brine (500 mL), dried over anhydrous sodium sulfate, filtered and the filtrate was concentrated under reduced pressure. The residue was purified by column chromatography to afford the title compound. ¹H NMR (400 MHz, CD₃C₁) 6=2.41-2.26 (m, 3H), 2.21-2.11 (m, 3H), 1.29-1.14 (m, 9H).

### Step 2: 2-methyl-N-(2-methylpent-4-en-2-yl)propane-2-sulfinamide

To a solution of allyl(chloro)magnesium (2 M in THF, 148.82 mL) in DMF (300 mL) was added 2-methyl-N-(propan-2-ylidene)propane-2-sulfinamide (32.0 g, 198.43 mmol) in THF (100 mL) at −65° C. After stirred at −65° C. for 1.5 h, the reaction mixture was quenched by addition of water (400 mL) and extracted with ethyl acetate (600 mL). The combined organic layers were washed with brine (500 mL), dried over anhydrous sodium sulfate, filtered and the filtrate was concentrated under reduced pressure. The residue was purified by column chromatography to afford the title compound. ¹H NMR (400 MHz, CD₃Cl) δ=5.96-5.71 (m, 1H), 5.24-4.89 (m, 2H), 3.26-2.94 (m, 1H), 2.36-2.21 (m, 2H), 1.32-1.28 (m, 3H), 1.26-1.23 (m, 3H), 1.20-1.15 (m, 8H).

### Step 3: 2-methyl-N-(2-methyl-1-(oxiran-2-yl)propan-2-yl)propane-2-sulfonamide

To a solution of 2-methyl-N-(2-methylpent-4-en-2-yl)propane-2-sulfinamide (31.0 g, 152.45 mmol) in dichloromethane (300 mL) was added meta-cholorperoxybenzoic acid (78.9 g, 457.35 mmol) at 0° C. After stirred at 20° C. for 16 h, the reaction mixture was quenched by addition of water (400 mL) and extracted with dichloromethane (600 mL). The combined organic layers were washed with brine (500 mL), dried over anhydrous sodium sulfate, filtered and the filtrate was concentrated under reduced pressure. The residue was purified by column chromatography to afford the title compound. ¹H NMR (400 MHz, CD₃Cl) δ=8.27-7.34 (m, 1H), 4.23-3.78 (m, 1H), 3.19-3.06 (m, 1H), 2.86-2.75 (m, 1H), 2.54-2.43 (m, 1H), 2.24-2.10 (m, 1H), 1.54-1.50 (m, 3H), 1.49-1.45 (m, 3H), 1.45-1.33 (m, 9H).

### Step 4: 1-(tert-butylsulfonyl)-5,5-dimethylpyrrolidin-3-ol

To a solution of 2-methyl-N-(2-methyl-1-(oxiran-2-yl)propan-2-yl)propane-2-sulfonamide (25.0 g, 106.23 mmol) in N, N-dimethylformamide (250 mL) was added potassium carbonate (44.1 g, 318.68 mmol) and potassium iodide (17.6 g, 106.23 mmol). The mixture was stirred at 100° C. for 3 h, the reaction mixture was quenched by addition of water (400 mL) and extracted with ethyl acetate (600 mL). The combined organic layers were washed with brine (500 mL), dried over anhydrous sodium sulfate, filtered and the filtrate was concentrated under reduced pressure. The residue was purified by column chromatography to afford the title compound. ¹H NMR (400 MHz, CD₃C₁) 6=4.54-4.33 (m, 1H), 3.90-3.70 (m, 1H), 3.53-3.24 (m, 1H), 2.19-2.07 (m, 1H), 1.76 (br. s, 2H), 1.65-1.55 (m, 3H), 1.52-1.46 (m, 3H), 1.45-1.36 (m, 9H).

### Step 5: 1-(tert-butylsulfonyl)-5,5-dimethylpyrrolidin-3-one

To a solution of 1-tert-butylsulfonyl-5,5-dimethyl-pyrrolidin-3-ol (17.5 g, 74.36 mmol) in dichloromethane (220 mL) was added Dessmartin periodide (23.4 g, 55.24 mmol) at 0° C. After stirred at 20° C. for 16 h, the reaction mixture was quenched by addition of water (100 mL) and extracted with dichloromethane (300 mL). The combined organic layers were washed with brine (300 mL), dried over anhydrous sodium sulfate, filtered and the filtrate was concentrated under reduced pressure. The residue was purified by column chromatography to afford the title compound. 1H NMR (400 MHz, CD₃C₁) 6=3.95-3.76 (m, 2H), 2.54-2.40 (m, 2H), 1.66-1.53 (m, 6H), 1.39 (s, 9H).

### Step 6: 1-(1-(tert-butylsulfonyl)-5,5-dimethylpyrrolidin-3-yl)-6-chloro-1,2,3,4-tetrahydroquinoline

To a solution of 1-tert-butylsulfonyl-5,5-dimethyl-pyrrolidin-3-one (5.0 g, 21.43 mmol) in acetic acid (50 mL) was added sodium borohydride acetate (13.6 g, 64.29 mmol) and 6-chloro-1,2,3,4-tetrahydroquinoline (5.4 g, 32.14 mmol). After stirred at 20° C. for 16 h, the reaction mixture was filtered and the filtrate was concentrated under reduced pressure. The residue was purified by preparative HPLC to afford the title compound. LCMS R_{T}=0.651 min, m/z=385.4 [M+H]⁺.

### Step 7: 8-bromo-1-(1-(tert-butylsulfonyl)-5,5-dimethylpyrrolidin-3-yl)-6-chloro-1,2,3,4-tetrahydroquinoline

To a solution of 1-(1-(tert-butylsulfonyl)-5,5-dimethylpyrrolidin-3-yl)-6-chloro-1,2,3,4-tetrahydroquinoline (1.9 g, 4.81 mmol) in dimethyl formamide (25 mL) was added 1-bromopyrrolidine-2,5-dione (86 mg, 0.480 mmol). After stirred at 20° C. for 1 h, the residue was purified by column chromatography to afford the title compound. LCMS R_{T}=1.568 min, m/z=465.2 [M+H]⁺.

### Step 8: 2-(((tert-butyldimethylsilyl)oxy)methyl)-7-(1-(1-(tert-butylsulfonyl)-5,5-dimethylpyrrolidin-3-yl)-6-chloro-1,2,3,4-tetrahydroquinolin-8-yl)thieno[3,2-b]pyridine

To a solution of 8-bromo-1-(1-(tert-butylsulfonyl)-5,5-dimethylpyrrolidin-3-yl)-6-chloro-1,2,3,4-tetrahydroquinoline (2.2 g, 4.74 mmol) in dioxane (50 mL) and water (5 mL) was added [2-[[tert-butyl(dimethyl)silyl]oxymethyl]thieno[3,2-b]pyridin-7-yl]boronic acid (1.8 g, 5.69 mmol), potassium carbonate (1.6 g, 11.86 mmol), [1,1′-Bis(diphenylphosphino)ferrocene]dichloropalladium(II) (694 mg, 0.949 mmol). After stirred at 100° C. for 3 h, the reaction mixture was filtered and the filtrate was concentrated under reduced pressure. The residue was purified by column chromatography to afford the title compound. LCMS R_{T}=1.158 min, m/z=663.8 [M+H]⁺.

### Step 9: 8-(2-(((tert-butyldimethylsilyl)oxy)methyl)thieno[3,2-b]pyridin-7-yl)-1-(1-(tert-butylsulfonyl)-5,5-dimethylpyrrolidin-3-yl)-1,2,3,4-tetrahydroquinoline-6-carbonitrile

To a solution of 2-(((tert-butyldimethylsilyl)oxy)methyl)-7-(1-(1-(tert-butylsulfonyl)-5,5-dimethylpyrrolidin-3-yl)-6-chloro-1,2,3,4-tetrahydroquinolin-8-yl)thieno[3,2-b]pyridine (3.2 g, 4.83 mmol) in dioxane (40 mL) was added N-ethyl-N-propan-2-ylpropan-2-amine (1.6 g, 12.08 mmol), zinc cyanide (1.1 g, 9.66 mmol) and palladium; tritert-butylphosphane (494 mg, 0.966 mmol). After stirred at 90° C. for 12 h, the reaction mixture was quenched by addition of water (150 mL) and extracted with ethyl acetate (200 mL). The combined organic layers were washed with brine (100 mL), dried over anhydrous sodium sulfate, filtered and the filtrate was concentrated under reduced pressure. The residue was purified by column chromatography to afford the title compound. LCMS R_{T}=0.63 min, m/z=653.8 [M+H]⁺.

### Step 10: (S)-1-(1-(tert-butylsulfonyl)-5,5-dimethylpyrrolidin-3-yl)-8-(2-(hydroxymethyl)thieno[3,2-b]pyridin-7-yl)-1,2,3,4-tetrahydroquinoline-6-carbonitrile and (R)-1-(1-(tert-butylsulfonyl)-5,5-dimethylpyrrolidin-3-yl)-8-(2-(hydroxymethyl)thieno[3,2-b]pyridin-7-yl)-1,2,3,4-tetrahydroquinoline-6-carbonitrile

To a solution of 8-(2-(((tert-butyldimethylsilyl)oxy)methyl)thieno[3,2-b]pyridin-7-yl)-1-(1-(tert-butylsulfonyl)-5,5-dimethylpyrrolidin-3-yl)-1,2,3,4-tetrahydroquinoline-6-carbonitrile (2.5 g, 3.83 mmol) in THF (25 mL) was added tetrabutylammonium fluoride (1 M in THF, 5.74 mL). After stirred at 90° C. for 12 h, the reaction mixture was quenched by addition of water (100 mL) and extracted with ethyl acetate (200 mL). The combined organic layers were washed with brine (100 mL), dried over anhydrous sodium sulfate, filtered and the filtrate was concentrated under reduced pressure. The residue was purified by column chromatography.

The racemate was separated by SFC to give first eluting fraction, 120-i (Rt=2.892 min, LCMS R_{T}-1.388 min, m/z=539.4 [M+H]+) and second eluting fraction, 120-ii (Rt=3.934 min, LCMS R_{T}=1.421 min, m/z=539.5 [M+H]⁺.

### Step 11

To a solution of 120-ii (30 mg, 0.055 mmol) in toluene (1.5 mL) was added 4-methyl-1H-pyridazin-6-one (18 mg, 0.167 mmol), tributylphosphane (68 mg, 0.334 mmol) and tetramethylazodicarboxamide (77 mg, 0.445 mmol) at 25° C. After stirred at 90° C. for 1 h, the reaction mixture was filtered and the filtrate was concentrated under reduced pressure. The residue was dissolved in dichloromethane (1.5 mL) was added trifluoromethanesulfonic acid (7 mg, 47.56 mmol) at 20° C. After stirred at 20° C. for 1 h, the reaction mixture was filtered and the filtrate was concentrated under reduced pressure. The residue was purified by preparative HPLC to afford Example 123a. ¹H NMR (400 MHz, CD₃OD) δ=8.82-8.69 (m, 1H), 8.54-8.42 (m, 1H), 7.87 (d, J=2.0 Hz, 1H), 7.65-7.41 (m, 4H), 6.85-6.72 (m, 1H), 5.71-5.47 (m, 2H), 4.09-3.78 (m, 1H), 3.30-2.72 (m, 6H), 2.33-2.18 (m, 3H), 2.07-1.16 (m, 7H), 0.91-0.22 (m, 3H). LCMS R_{T}=0.427 min, m/z=511.3 [M+H]+

Example 123b was prepared from 123-i, following the procedure described above. The product was purified by preparative HPLC. ¹H NMR (400 MHz, CD₃OD) δ=8.77 (d, J=3.6 Hz, 1H), 8.65-8.47 (m, 1H), 7.89 (d, J=2.0 Hz, 1H), 7.73-7.32 (m, 4H), 6.81 (s, 1H), 5.61 (d, J=6.8 Hz, 2H), 4.05-3.77 (m, 1H), 3.42-3.15 (m, 2H), 3.13-2.80 (m, 3H), 2.79-2.49 (m, 1H), 2.35-2.21 (m, 3H), 2.10-1.73 (m, 3H), 1.51-1.33 (m, 4H), 0.76-0.10 (m, 3H). LCMS R_{T}=1.043 min, m/z=511.4 [M+H]⁺.

The absolute configuration was tentatively assigned based on the relative potency of the pair diastereomers in biology assays.

### Example 121: (S)-1-(5,5-dimethylpyrrolidin-3-yl)-8-(2-((3-methyl-2,6-dioxo-3,6-dihydropyrimidin-1(2H)-yl)methyl)thieno[3,2-b]pyridin-7-yl)-1,2,3,4-tetrahydroquinoline-6-carbonitrile, formic acid salt

Prepared from 120-ii and 1-methylpyrimidine-2,4-dione, following the procedure described in the synthesis of Example 108a. The residue was purified by preparative HPLC. ¹H NMR (400 MHz, CD₃OD) δ=8.74 (s, 1H), 8.51 (s, 1H), 7.61-7.55 (m, 2H), 7.49 (d, J=6.4 Hz, 3H), 5.79-5.70 (m, 1H), 5.38 (s, 2H), 4.03-3.83 (m, 1H), 3.38 (s, 3H), 3.28-3.16 (m, 1H), 3.12-2.59 (m, 4H), 1.96 (d, J=6.2 Hz, 3H), 1.76-1.19 (m, 5H), 0.86-0.23 (m, 4H). LCMS: R_{T}=0.930 min, m/z=527.4 [M+H]⁺.

### Example 122: (S)-8-(2-((4-methyl-6-oxopyridazin-1(6H)-yl)methyl)thieno[3,2-b]pyridin-7-yl)-1-(1,5,5-trimethylpyrrolidin-3-yl)-1,2,3,4-tetrahydroquinoline-6-carbonitrile, formic acid salt

To a solution of Example 120a (20 mg, 0.039 mmol) in acetonitrile (1 mL) was added triethylamine (8 mg, 0.078 mmol), sodium borohydride acetate (17 mg, 0.078 mmol) and formaldehyde (3 mg, 0.078 mmol) at 20° C. After stirred at 20° C. for 1 h, the reaction mixture was filtered and the filtrate was concentrated under reduced pressure. The residue was purified by preparative HPLC to afford the title compound. ¹H NMR (400 MHz, CD₃OD) δ=8.81-8.65 (m, 1H), 8.58-8.34 (m, 1H), 7.87 (d, J=1.6 Hz, 1H), 7.61-7.55 (m, 1H), 7.51-7.42 (m, 3H), 6.83-6.76 (m, 1H), 5.65-5.53 (m, 2H), 4.05-3.73 (m, 1H), 3.28-2.33 (m, 7H), 2.25 (d, J=0.8 Hz, 4H), 2.13-1.37 (m, 4H), 1.28-1.02 (m, 3H), 0.76-0.01 (m, 4H). LCMS R_{T}=1.025 min, m/z=525.4 [M+H]⁺.

### Example 123: (S)-8-(2-((3-methyl-2,6-dioxo-2,3-dihydropyrimidin-1(6H)-yl)methyl)thieno[3,2-b]pyridin-7-yl)-1-(1,5,5-trimethylpyrrolidin-3-yl)-1,2,3,4-tetrahydroquinoline-6-carbonitrile, formic acid salt

To a solution of Example 121 (40 mg, 0.076 mmol) in acetonitrile (2 mL) was added formaldehyde (12 mg, 0.15 mmol, 37% purity), sodium triacetoxyborohydride (32 mg, 0.15 mmol) and N,N-diethylethanamine (15 mg, 0.15 mmol) at 20° C. After stirred at 20° C. for 1 h, the reaction mixture was concentrated under reduced pressure. The residue was purified by preparative HPLC to afford the title compound. ¹H NMR (400 MHz, CD₃OD) δ=8.76-8.69 (m, 1H), 8.49-8.43 (m, 1H), 7.62-7.53 (m, 2H), 7.51-7.42 (m, 3H), 5.78-5.72 (m, 1H), 5.38 (s, 2H), 3.95-3.78 (m, 1H), 3.40-3.37 (m, 3H), 3.27-3.15 (m, 1H), 2.96-2.81 (m, 2H), 2.80-1.80 (m, 7H), 1.69-0.97 (m, 5H), 0.76-0.08 (m, 4H). LCMS R_{T}=0.930 min, m/z=541.4 [M+H]⁺.

### Example 124: 1-((S)-5,5-dimethylpyrrolidin-3-yl)-8-(2-((2,4-dioxo-3-azabicyclo[3.1.0]hexan-3-yl)methyl)thieno[3,2-b]pyridin-7-yl)-1,2,3,4-tetrahydroquinoline-6-carbonitrile, formic acid salt

Example 124 was prepared from 120-ii and 3-azabicyclo[3.1.0]hexane-2,4-dione, following the procedure described in the synthesis of Example 123a. The product was purified by preparative HPLC. ¹H NMR (400 MHz, CD₃OD) δ=8.75 (s, 1H), 8.46 (s, 1H), 7.56-7.41 (m, 4H), 4.81-4.74 (m, 2H), 4.03-3.84 (m, 1H), 3.10 (dd, J=2.4, 7.2 Hz, 1H), 3.00-2.70 (m, 3H), 2.57 (dd, J=3.6, 8.0 Hz, 2H), 2.06-1.22 (m, 10H), 0.86-0.27 (m, 3H)). LCMS R_{T}=0.993 min, m/z=512.4 [M+H]⁺.

### Example 125: (S)-1-(5,5-dimethylpyrrolidin-3-yl)-8-(2-((2,6-dioxo-3-(2,2,2-trifluoroethyl)-2,3-dihydropyrimidin-1(6H)-yl)methyl)thieno[3,2-b]pyridin-7-yl)-1,2,3,4-tetrahydroquinoline-6-carbonitrile, formic acid salt

Example 125 was prepared from 120-ii and 1-(2,2,2-trifluoroethyl)pyrimidine-2,4-dione, following the procedure described in the synthesis of Example 123a. The product was purified by preparative HPLC. ¹H NMR (400 MHz, CD₃OD) δ=8.79-8.70 (m, 1H), 8.57-8.43 (m, 1H), 7.69-7.62 (m, 1H), 7.61-7.42 (m, 4H), 5.92-5.82 (m, 1H), 5.44-5.34 (m, 2H), 4.62 (br. d, J=8.8 Hz, 2H), 4.10-3.82 (m, 1H), 3.26-2.58 (m, 4H), 2.01-1.16 (m, 8H), 0.83-0.30 (m, 4H). LCMS R_{T}=1.164 min, m/z=595.4 [M+H]⁺.

### Example 126: (S)-8-(2-((4-(dimethylamino)-3-ethyl-2,6-dioxo-2,3-dihydropyrimidin-1(6H)-yl)methyl)thieno[3,2-b]pyridin-7-yl)-1-(5,5-dimethylpyrrolidin-3-yl)-1,2,3,4-tetrahydroquinoline-6-carbonitrile, formic acid salt

Example 126 was prepared from 120-ii and 6-(dimethylamino)-1-ethyl-pyrimidine-2,4-dione, following the procedure described in the synthesis of Example 123a. The product was purified by preparative HPLC. ¹H NMR (400 MHz, CD₃OD) δ=8.77-8.69 (m, 1H), 8.53-8.46 (m, 1H), 7.62-7.51 (m, 1H), 7.51-7.42 (m, 3H), 5.39-5.28 (m, 2H), 5.26-5.21 (m, 1H), 4.05-3.85 (m, 3H), 3.24-2.81 (m, 4H), 2.81-2.57 (m, 7H), 2.07-1.69 (m, 3H), 1.59-1.44 (m, 1H), 1.33-1.20 (m, 6H), 0.81-0.25 (m, 4H). LCMS: R_{T}=0.433 min, m/z=584.4 [M+H]⁺.

### Example 127: (S)-8-(2-((3-(3,3-difluorocyclobutyl)-2,5-dioxoimidazolidin-1-yl)methyl)thieno[3,2-b]pyridin-7-yl)-1-(5,5-dimethylpyrrolidin-3-yl)-1,2,3,4-tetrahydroquinoline-6-carbonitrile, formic acid salt

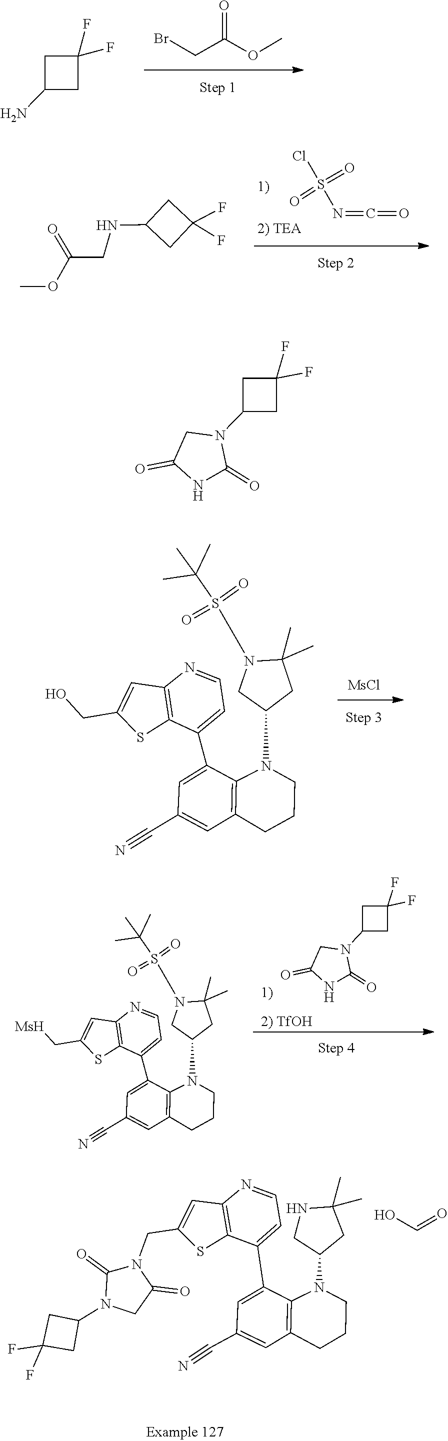

### Step 1: methyl 2-((3,3-difluorocyclobutyl)amino)acetate

To a solution of 3,3-difluorocyclobutanamine (73 mg, 0.5 mmol) and methyl 2-bromoacetate (70 mg, 0.45 mmol) in ethanol (1 mL) was added triethylamine (93 mg, 0.9 mmol) at 20° C. After stirred at 20° C. for 12 h, the reaction mixture was quenched by addition of saturated aqueous sodium bicarbonate (5 mL), the residue was extracted by ethyl acetate (5 mL×4) and water (2 mL×4), and dried over anhydrous sodium sulfate, filtered and the filtrate was concentrated under reduced pressure to afford the title compound which was used in next step without further purification. ¹H NMR (400 MHz, CDCl₃) δ=3.74-3.61 (m, 3H), 3.32-3.27 (m, 2H), 3.24-3.12 (m, 1H), 2.80-2.60 (m, 2H), 2.35-2.16 (m, 2H).

### Step 2: 1-(3,3-difluorocyclobutyl)imidazolidine-2,4-dione

To a solution of methyl 2-[(3,3-difluorocyclobutyl)amino]acetate (300 mg, 1.67 mmol) in dichloromethane (1 mL) was added N-(oxomethylene)sulfamoyl chloride (355 mg, 2.51 mmol) at 0° C. After stirred at 0° C. for 2 h, the mixture was added water (1 mL). After stirred at 20° C. for 12 h, the reaction mixture was quenched by addition of saturated aqueous sodium bicarbonate (5 mL), the residue was extracted by ethyl acetate (5 mL×4) and water (2 mL×4), and dried over anhydrous sodium sulfate, filtered and the filtrate was concentrated under reduced pressure. The residue was purified by preparative TLC to afford the title compound. ¹H NMR (400 MHz, CDCl₃) δ=4.66-4.28 (m, 1H), 3.91 (s, 2H), 2.98-2.82 (m, 2H), 2.78-2.55 (m, 2H)

### Step 3: (S)-(7-(1-(1-(tert-butylsulfonyl)-5,5-dimethylpyrrolidin-3-yl)-6-cyano-1,2,3,4-tetrahydroquinolin-8-yl)thieno[3,2-b]pyridin-2-yl)methyl methanesulfonate

To a solution of (S)-1-(1-(tert-butylsulfonyl)-5,5-dimethylpyrrolidin-3-yl)-8-(2-(hydroxymethyl)thieno[3,2-b]pyridin-7-yl)-1,2,3,4-tetrahydroquinoline-6-carbonitrile (50 mg, 0.09 mmol) in Methylene chloride (1 mL) was added methanesulfonyl chloride (280 mg, 2.51 mmol) and trimethylamine (30 mg, 0.28 mmol) at 0° C. After stirred at 25° C. for 1 h, the reaction mixture was quenched with saturated aqueous sodium bicarbonate (5 mL), the residue was extracted by Methylene chloride (5 mL×4) and water (2 mL×4), and dried over anhydrous sodium sulfate, filtered and the filtrate was concentrated under reduced pressure to afford the title compound, which was used in next step without further purification. LCMS R_{T}=1.498, m/z=617.2 [M+H]⁺.

### Step 4

To a solution of (S)-(7-(1-(1-(tert-butylsulfonyl)-5,5-dimethylpyrrolidin-3-yl)-6-cyano-1,2,3,4-tetrahydroquinolin-8-yl)thieno[3,2-b]pyridin-2-yl)methyl methanesulfonate (80 mg, 0.13 mmol) in DMF (2 mL) was added 1-(3,3-difluorocyclobutyl)imidazolidine-2,4-dione (40 mg, 0.19 mmol), 2,3,4,6,7,8,9,10-octahydropyrimido[1,2-a]azepine (20 mg, 0.13 mmol) at 25° C. After stirred at 25° C. for 2 h, the reaction mixture was concentrated under reduced pressure. The residue was dissolved in methylene chloride (1 mL) and trifluoroacetic acid (0.02 mL, 0.07 mmol) was added at 25° C. After stirred at 25° C. for 2 h, the reaction mixture was concentrated under reduced pressure. The residue was purified by preparative HPLC to afford Example 127. ¹H NMR (400 MHz, CD₃OD) δ=8.67-8.83 (m, 1H), 8.52 (s, 1H), 7.51 (d, J=19.6 Hz, 4H), 4.94-5.00 (m, 2H), 4.40-4.50 (m, 1H), 4.18-4.22 (m, 2H), 4.05-4.21 (m, 1H), 3.87-4.03 (m, 1H), 2.81-3.00 (m, 6H), 2.81-3.14 (m, 1H), 2.59-2.76 (m, 1H), 1.43-1.79 (m, 1H), 1.21-1.32 (m, 2H), 1.12-2.07 (m, 4H), 0.23-0.90 (m, 4H). LCMS R_{T}=0.453, m/z=591.3 [M+H]⁺.

### Example 128: (S)-8-(2-((4-cyclopropyl-6-oxopyridazin-1(6H)-yl)methyl)thieno[3,2-b]pyridin-7-yl)-1-(5,5-dimethylpyrrolidin-3-yl)-1,2,3,4-tetrahydroquinoline-6-carbonitrile, formic acid salt

Example 128 was prepared from 123-ii and 4-cyclopropyl-1H-pyridazin-6-one, following the procedure described in the synthesis of Example 123a. The product was purified by preparative HPLC. ¹H NMR (400 MHz, CD₃OD) δ=8.85-8.65 (m, 1H), 8.55-8.38 (m, 1H), 7.85-7.72 (m, 1H), 7.63-7.36 (m, 4H), 6.69-6.43 (m, 1H), 5.72-5.37 (m, 2H), 4.08-3.76 (m, 1H), 3.21 (br. s, 5H), 2.11-1.64 (m, 4H), 1.55-0.97 (m, 6H), 0.93-0.84 (m, 2H), 0.79-0.20 (m, 4H). LCMS R_{T}=1.147 min, m/z=537.4 [M+H]⁺.

### Example 129: (S)-1-(5,5-dimethylpyrrolidin-3-yl)-8-(2-((4-isopropyl-6-oxopyridazin-1(6H)-yl)methyl)thieno[3,2-b]pyridin-7-yl)-1,2,3,4-tetrahydroquinoline-6-carbonitrile

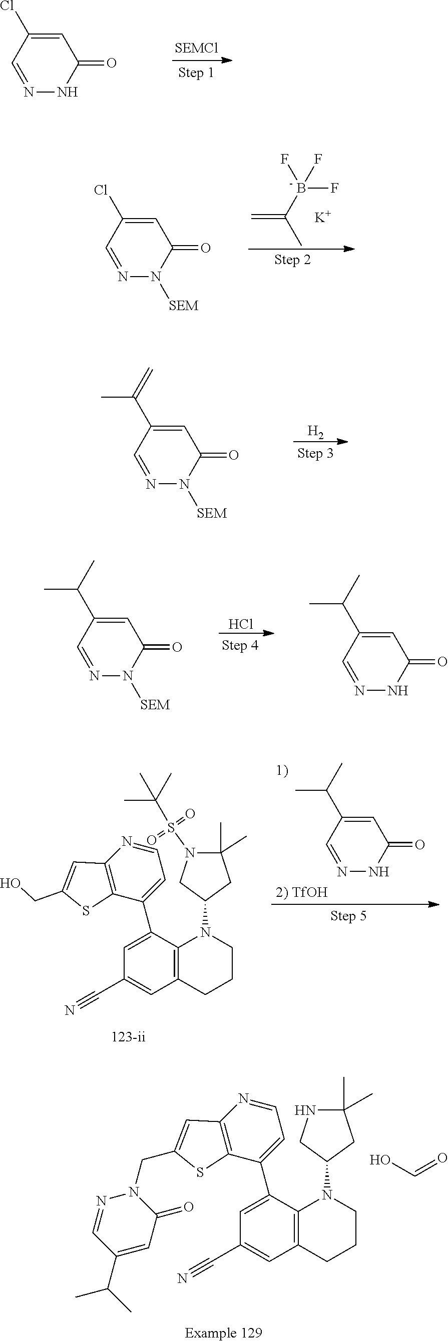

### Step 1: 5-chloro-2-((2-(trimethylsilyl)ethoxy)methyl)pyridazin-3(2H)-one

To a solution of 5-chloropyridazin-3(2H)-one (2.0 g, 15.32 mmol) in N,N-dimethylacetamide (20 mL) was added sodium hydride (919 mg, 22.98 mmol). After stirred at 25° C. for 0.5 h under nitrogen atmosphere, the mixture was added (2-(chloromethoxy)ethyl)trimethylsilane (3.8 g, 22.98 mmol). After stirred at 25° C. for 16 h under nitrogen atmosphere, the reaction mixture was quenched by addition of water (50 mL), and extracted with ethyl acetate (20 mL×2). The combined organic layers were washed with brine (20 mL), dried over anhydrous sodium sulfate, filtered and the filtrate was concentrated under reduced pressure. The residue was purified by column chromatography to afford the title compound. ¹H NMR (400 MHz, CDCl₃) δ=7.80-7.71 (m, 1H), 7.02-6.95 (m, 1H), 5.42 (s, 2H), 3.74-3.66 (m, 2H), 0.99-0.94 (m, 2H), 0.02 (s, 9H).

### Step 2: 5-(prop-1-en-2-yl)-2-((2-(trimethylsilyl)ethoxy)methyl)pyridazin-3(2H)-one

To a solution of 5-chloro-2-((2-(trimethylsilyl)ethoxy)methyl)pyridazin-3(2H)-one (200 mg, 0.77 mmol) in dioxane (1.6 mL) and water (0.4 mL) was added potassium trifluoro(prop-1-en-2-yl)borate (170 mg, 1.15 mmol), cesium carbonate (750 mg, 2.30 mmol) and XPhos Pd G3 (32 mg, o.o4 mmol). After stirred at 100° C. for 3 h under nitrogen atmosphere under microwave, the reaction mixture was filtered and the filtrate was concentrated under reduced pressure, extracted with ethyl acetate (20 mL×2). The combined organic layers were washed with brine (20 mL), dried over anhydrous sodium sulfate, filtered and the filtrate was concentrated under reduced pressure. The residue was purified by preparative TLC to afford the title compound. ¹H NMR (400 MHz, CDCl₃) δ=8.02-7.95 (m, 1H), 6.85-6.79 (m, 1H), 5.70-5.63 (m, 1H), 5.50-5.41 (m, 3H), 3.76-3.67 (m, 2H), 2.11-2.05 (m, 3H), 1.02-0.94 (m, 2H), 0.04-0.00 (m, 9H).

### Step 3: 5-isopropyl-2-((2-(trimethylsilyl)ethoxy)methyl)pyridazin-3(2H)-one

To a solution of 5-(prop-i-en-2-yl)-2-((2-(trimethylsilyl)ethoxy)methyl)pyridazin-3(2H)-one (200 mg, 0.75 mmol) in methyl alcohol (2 mL) was added 10% palladium on carbon (10 mg). After stirred at 20° C. for 2 h under hydrogen atmosphere (15 psi), the reaction mixture was filtered and the filtrate was concentrated under reduced pressure to afford the title compound. It was used in the next step without further purification. ¹H NMR (400 MHz, CDCl₃) δ=7.72-7.68 (m, 1H), 6.76-6.68 (m, 1H), 5.46 (s, 2H), 3.75-3.67 (m, 2H), 2.84-2.70 (m, 1H), 1.26-1.23 (m, 6H), 1.00-0.95 (m, 2H), 0.02-0.04 (m, 9H).

### Step 4: 5-isopropylpyridazine-3(2H)-one

To a solution of 5-isopropyl-2-((2-(trimethylsilyl)ethoxy)methyl)pyridazin-3(2H)-one (150 mg, 0.56 mmol) in methyl alcohol (4 mL) was added hydrochloric acid (0.5 mL, 12 M). After stirred at 90° C. for 4 h, the reaction mixture was filtered and the filtrate was concentrated under reduced pressure. The residue was purified by preparative HPLC to afford the title compound. ¹H NMR (400 MHz, CDCl₃) δ=11.13-10.44 (m, 1H), 7.76-7.67 (m, 1H), 6.77-6.70 (m, 1H), 2.86-2.73 (m, 1H), 1.29-1.22 (m, 6H).

### Step 5: (S)-1-(5,5-dimethylpyrrolidin-3-yl)-8-(2-((4-isopropyl-6-oxopyridazin-1(6H)-yl)methyl)thieno[3,2-b]pyridin-7-yl)-1,2,3,4-tetrahydroquinoline-6-carbonitrile, formic acid salt

Example 129 was prepared from 123-ii and 4-isopropyl-1H-pyridazin-6-one, following the procedure described in the synthesis of Example 123a. The product was purified by preparative HPLC. ¹H NMR (400 MHz, CD₃OD) δ=8.80 (s, 1H), 8.53-8.45 (m, 1H), 7.96 (d, J=2.4 Hz, 1H), 7.63-7.55 (m, 1H), 7.54-7.44 (m, 3H), 6.80-6.73 (m, 1H), 5.68-5.53 (m, 2H), 4.05-3.79 (m, 1H), 3.27-3.03 (m, 2H), 2.97-2.80 (m, 3H), 2.75-2.58 (m, 1H), 2.04-1.67 (m, 3H), 1.60-1.40 (m, 1H), 1.35-1.29 (m, 1H), 1.28-1.17 (m, 8H), 0.96-0.26 (m, 4H). LCMS: R_{T}=0.468 min, m/z=539.3 [M+H]⁺.

### Example 130: (S)-1-(5,5-dimethylpyrrolidin-3-yl)-8-(2-((2,6-dioxopiperidin-1-yl)methyl)thieno[3,2-b]pyridin-7-yl)-1,2,3,4-tetrahydroquinoline-6-carbonitrile, formic acid salt

Example 130 was prepared from 123-ii and piperidine-2,6-dione (13 mg, 0.011 mmol), following the procedure described in the synthesis of Example 123a. The product was purified by preparative HPLC. ¹H NMR (400 MHz, CD₃OD) δ=8.79-8.70 (m, 1H), 8.55-8.47 (m, 1H), 7.55-7.43 (m, 4H), 5.27-5.16 (m, 2H), 4.05-3.86 (m, 1H), 3.29-3.01 (m, 2H), 3.00-2.83 (m, 2H), 2.79-2.62 (m, 5H), 2.03-1.66 (m, 5H), 1.61-1.45 (m, 1H), 1.37-1.22 (m, 3H), 1.06-0.13 (m, 4H). LCMS: R_{T}=0.960 min, m/z=514.5 [M+H]⁺.

### Example 131: (S)-8-(2-((3-cyclopropyl-2,5-dioxoimidazolidin-1-yl)methyl)thieno[3,2-b]pyridin-7-yl)-1-(5,5-dimethylpyrrolidin-3-yl)-1,2,3,4-tetrahydroquinoline-6-carbonitrile, formic acid salt

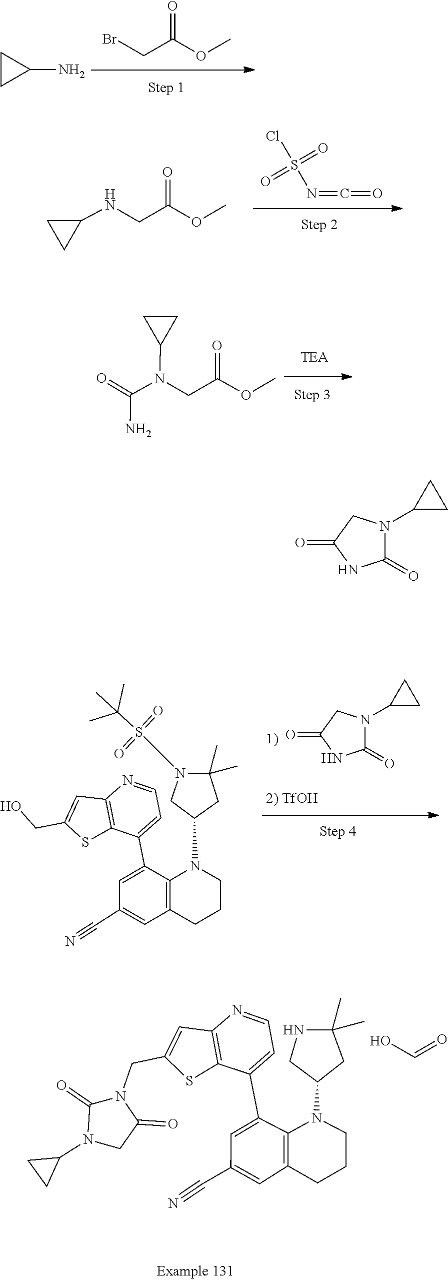

### Step 1: methyl 2-(cyclopropylamino)acetate

To a solution of cyclopropanamine (10.0 g, 175.15 mmol) and trimethylamine (35.5 g, 350.30 mmol) in methyl alcohol (70 mL) was added methyl 2-bromoacetate (24.1 g, 157.63 mmol) at 0° C. After stirred at 25° C. for 12 h, the reaction mixture was quenched with water (50 mL), the residue was extracted by ethyl acetate (20 mL×4) and water (10 mL×4), and dried over anhydrous sodium sulfate, filtered and the filtrate was concentrated under reduced pressure. The residue was purified by column chromatography to afford the title compound. ¹H NMR (400 MHz, CDCl₃) δ=0.25-0.50 (m, 4H) 1.16-1.32 (m, 1H) 2.03 (s, 1H) 2.16 (s, 1H) 2.19-2.27 (m, 1H) 3.46 (s, 2H) 3.70-3.75 (m, 3H).

### Step 2: methyl 2-(1-cyclopropylureido)acetate

To a solution of methyl 2-(cyclopropylamino) acetate (1.0 g, 7.74 mmol) in methylene chloride (10 mL) was added N-(oxomethylene) sulfamoyl chloride (1.1 g, 7.74 mmol) at 0° C. After stirred for 2 h, the mixture was added water (10 mL) at 0° C. After stirred at 25° C. for 12 h, the reaction mixture was concentrated under reduced pressure. The residue was purified by preparative TLC to afford the title compound. ¹H NMR (400 MHz, CDCl₃) δ=0.72-0.97 (m, 4H) 2.58-2.85 (m, 1H) 3.56-3.82 (m, 3H) 4.03-4.17 (m, 2H) 5.18 (s, 2H).

### Step 3: 1-cyclopropylimidazolidine-2,4-dione

To a solution of methyl 2-[carbamoyl(cyclopropyl) amino]acetate (280 mg, 1.63 mmol) in methyl alcohol (5 mL) was added trimethylamine (0.4 mL, 2.44 mmol) at 25° C. After stirred at 25° C. for 12 h, the reaction mixture was concentrated under reduced pressure. The residue was purified by column chromatography to afford the title compound. ¹H NMR (400 MHz, CDCl₃) δ=0.77-0.88 (m, 4H) 2.59-2.66 (m, 1H) 3.89 (s, 2H) 7.92-8.21 (m, 1H).

### Step 4

Example 131 was prepared from 123-ii and 1-cyclopropylimidazolidine-2,4-dione (10 mg, 0.08 mmol), following the procedure described in the synthesis of Example 123a. The product was purified by preparative HPLC. ¹H NMR (400 MHz, CD₃OD) δ=8.72-8.78 (m, 1H), 8.49 (s, 1H), 7.45-7.54 (m, 4H), 4.93 (s, 2H), 3.92-4.03 (m, 3H), 2.56-3.27 (m, 5H), 1.82-2.07 (m, 3H), 1.15-1.74 (m, 5H), 0.37-0.83 (m, 8H). LCMS R_{T}=0.987, m/z=541.4 [M+H]⁺.

### Example 132: (S)-8-(2-((4-(tert-butyl)-6-oxopyridazin-1(6H)-yl)methyl)thieno[3,2-b]pyridin-7-yl)-1-(5,5-dimethylpyrrolidin-3-yl)-1,2,3,4-tetrahydroquinoline-6-carbonitrile, formic acid salt

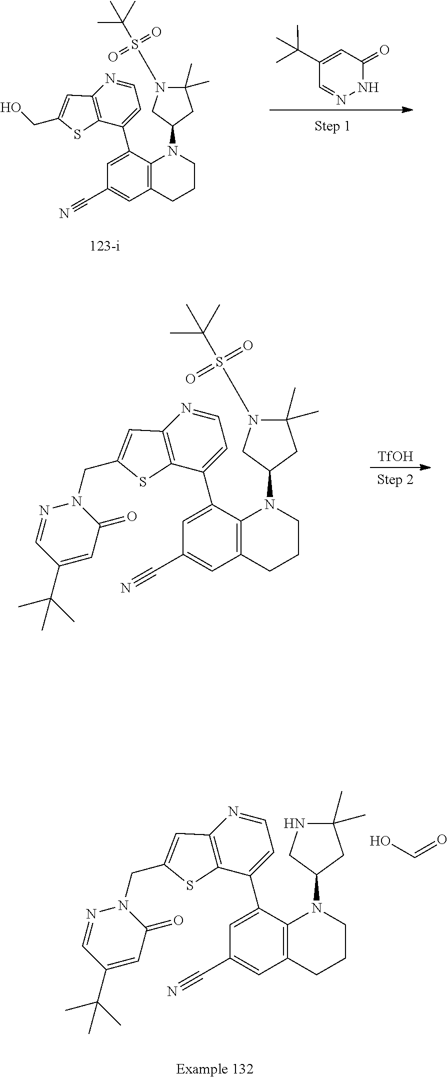

### Step 1: (S)-8-(2-((4-(tert-butyl)-6-oxopyridazin-1(6H)-yl)methyl)thieno[3,2-b]pyridin-7-yl)-1-(1-(tert-butylsulfonyl)-5,5-dimethylpyrrolidin-3-yl)-1,2,3,4-tetrahydroquinoline-6-carbonitrile

To the solution of (S)-1-(1-(tert-butylsulfonyl)-5,5-dimethylpyrrolidin-3-yl)-8-(2-(hydroxymethyl)thieno[3,2-b]pyridin-7-yl)-1,2,3,4-tetrahydroquinoline-6-carbonitrile (123-i, 30 mg, 0.06 mmol) in toluene (1 mL) was added 5-(tert-butyl)pyridazin-3(2H)-one (13 mg, 0.08 mmol), tetramethylazodicarboxamide (19 mg, 0.11 mmol) and tributylphosphane (45 mg, 0.22 mmol). After stirred at 95° C. for 1 h under nitrogen atmosphere, the reaction mixture was filtered and the filtrate was concentrated under reduced pressure. The residue was purified by preparative TLC to afford the title compound. LCMS R_{T}=1.860 min, m/z=673.4 [M+H]⁺.

### Step 2

To the solution of (S)-8-(2-((4-(tert-butyl)-6-oxopyridazin-1(6H)-yl)methyl)thieno[3,2-b]pyridin-7-yl)-1-(1-(tert-butylsulfonyl)-5,5-dimethylpyrrolidin-3-yl)-1,2,3,4-tetrahydroquinoline-6-carbonitrile (48 mg, 0.07 mmol) in dichloromethane (1 mL) was added trifluoromethanesulfonic acid (96 mg, 0.64 mmol). After stirred at 20° C. for 0.5 h, the reaction mixture was concentrated under reduced pressure to remove solution. The residue was purified by preparative HPLC to afford Example 132. ¹H NMR (400 MHz, CD₃OD) δ=8.84-8.65 (m, 1H), 8.56-8.83 (m, 1H), 8.18-8.08 (m, 1H), 7.65-7.38 (m, 4H), 6.88-6.75 (m, 1H), 5.68-5.52 (m, 2H), 4.06-3.77 (m, 1H), 3.29-2.54 (m, 6H), 2.04-1.66 (m, 3H), 1.57-1.38 (m, 1H), 1.33-1.17 (m, 12H), 0.87-0.48 (m, 3H). LCMS R_{T}=1.298 min, m/z=553.4 [M+H]⁺.

### Example 133a and 133b: (S)-4-(5,5-dimethylpyrrolidin-3-yl)-5-(2-((2,6-dioxo-3-(2,2,2-trifluoroethyl)-2,3-dihydropyrimidin-1(6H)-yl)methyl)thieno[3,2-b]pyridin-7-yl)-3,4-dihydro-2H-benzo[b][1,4]oxazine-7-carbonitrile, formic acid salt and (R)-4-(5,5-dimethylpyrrolidin-3-yl)-5-(2-((2,6-dioxo-3-(2,2,2-trifluoroethyl)-2,3-dihydropyrimidin-1(6H)-yl)methyl)thieno[3,2-b]pyridin-7-yl)-3,4-dihydro-2H-benzo[b][1,4]oxazine-7-carbonitrile, formic acid salt

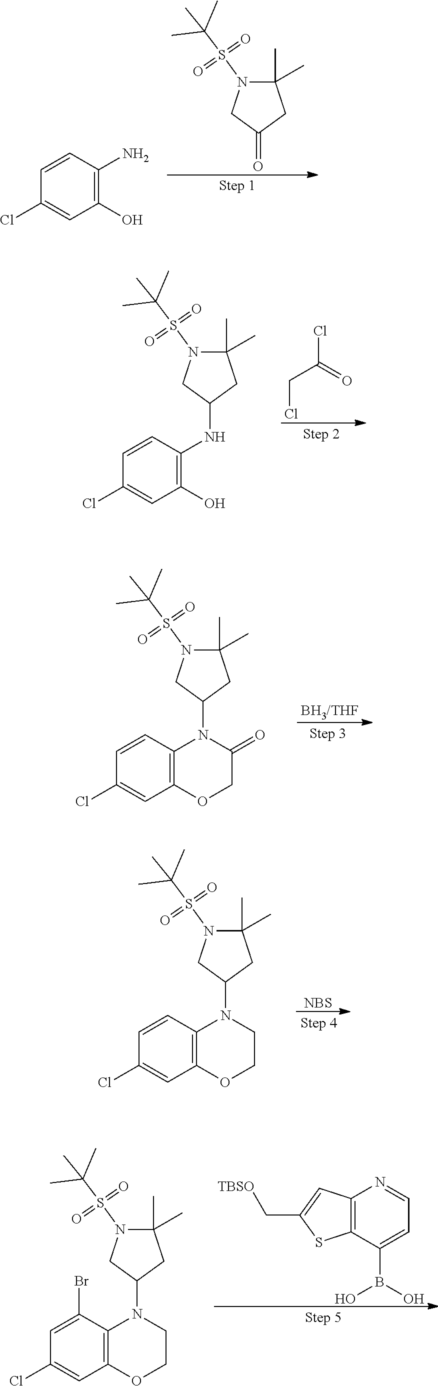

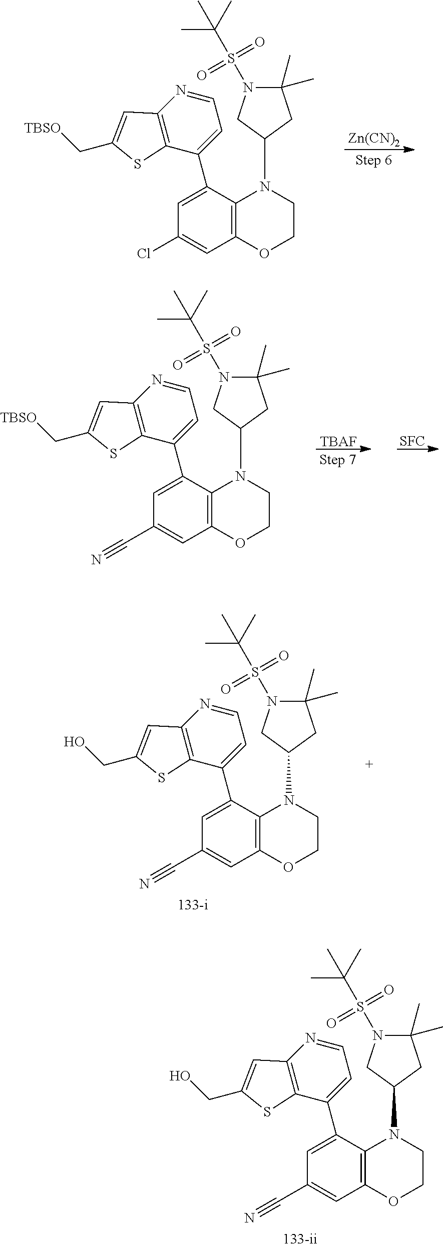

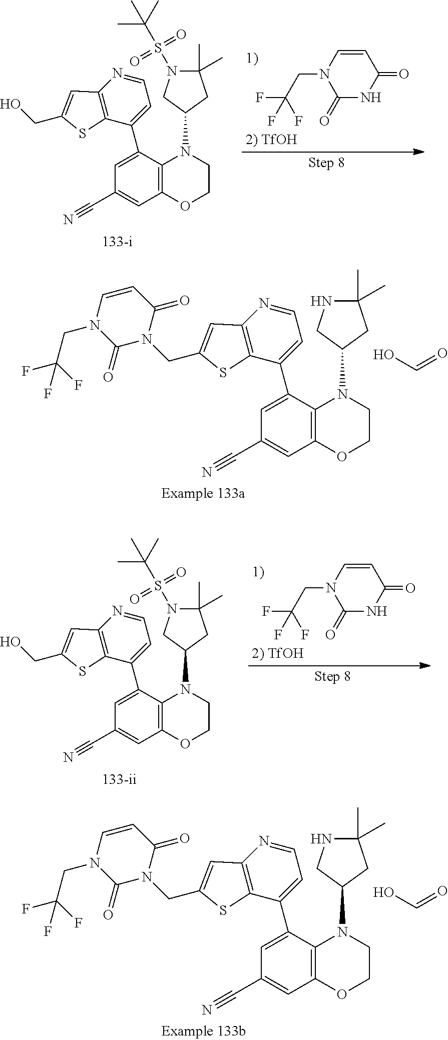

### Step 1: 2-[(1-tert-butylsulfonyl-5,5-dimethyl-pyrrolidin-3-yl)amino]-5-chloro-phenol

To a solution of 2-amino-5-chloro-phenol (4.0 g, 27.86 mmol) in 1,2-dichloroethane (30 mL) were added hydrochloric acid (4 M in methyl alcohol, 0.58 mL), 1-tert-butylsulfonyl-5,5-dimethyl-pyrrolidin-3-one (5.4 g, 23.22 mmol) and sodium triacetoxyborohydride (9.8 g, 46.43 mmol) at 25° C. After stirred at 25° C. for 2 h, the reaction mixture was diluted with water (200 mL) and extracted with dichloromethane (150 mL×3). The combined organic layers were washed with brine (250 mL), dried over anhydrous sodium sulfate, filtered and the filtrate was concentrated under reduced pressure. The residue was purified by column chromatography to afford the title compound. LCMS R_{T}=1.245 min, m/z=361.2 [M+H]⁺.

### Step 2: 4-(1-(tert-butylsulfonyl)-5,5-dimethylpyrrolidin-3-yl)-7-chloro-2H-benzo[b][1,4]oxazin-3(4H)-one

To a solution of 2-[(1-tert-butylsulfonyl-5,5-dimethyl-pyrrolidin-3-yl)amino]-5-chloro-phenol (7.7 g, 21.34 mmol) in acetonitrile (100 mL) were added 2-chloroacetyl chloride (3.6 g, 32 mmol) and potassium carbonate aqueous solution (3.75 M, 19.91 mL) at 25° C. After stirred at 80° C. for 2 h, the reaction mixture was concentrated under reduced pressure. Then the mixture was diluted with water (100 mL) and extracted with dichloromethane (100 mL×3). The combined organic layers were washed with brine (150 mL), dried over anhydrous sodium sulfate, filtered and the filtrate was concentrated under reduced pressure. The residue was purified by column chromatography to afford the title compound. LCMS R_{T}=1.245 min, m/z=361.2 [M+H]⁺.

### Step 3: 4-(1-(tert-butylsulfonyl)-5,5-dimethylpyrrolidin-3-yl)-7-chloro-3,4-dihydro-2H-benzo[b][1,4]oxazine

To a solution of 4-(1-tert-butylsulfonyl-5,5-dimethyl-pyrrolidin-3-yl)-7-chloro-1,4-benzoxazin-3-one (10.4 g, 25.94 mmol) in THF (70 mL) was added borane-THF (1 M, 77.82 mL). After stirred at 60° C. for 3 h, the reaction mixture was quenched by addition of methyl alcohol (50 mL) and concentrated under reduced pressure. The residue was purified by column chromatography to afford the title compound. LCMS R_{T}=1.418 min, m/z=387.2 [M+H]⁺.

### Step 4: 5-bromo-4-(1-(tert-butylsulfonyl)-5,5-dimethylpyrrolidin-3-yl)-7-chloro-3,4-dihydro-2H-benzo[b][1,4]oxazine

To a solution of 4-(1-tert-butylsulfonyl-5,5-dimethyl-pyrrolidin-3-yl)-7-chloro-2,3-dihydro-1,4-benzoxazine (2.0 g, 5.17 mmol) in dichloromethane (40 mL) was added N-bromosuccinimide (1.0 g, 5.69 mmol). After stirred at 0° C. for 1 h, the reaction mixture was concentrated under reduced pressure. Then the mixture was diluted with water (50 mL), extracted with ethyl acetate (50 mL×3). The combined organic layers were washed with brine (100 mL), dried over anhydrous sodium sulfate and concentrated under reduced pressure. The residue was purified by column chromatography to afford the title compound. LCMS R_{T}=0.662 min, m/z=467.0 [M+H]⁺.

### Step 5: 5-(2-(((tert-butyldimethylsilyl)oxy)methyl)thieno[3,2-b]pyridin-7-yl)-4-(1-(tert-butylsulfonyl)-5,5-dimethylpyrrolidin-3-yl)-7-chloro-3,4-dihydro-2H-benzo[b][1,4]oxazine

To a solution of 5-bromo-4-(1-tert-butylsulfonyl-5,5-dimethyl-pyrrolidin-3-yl)-7-chloro-2,3-dihydro-1,4-benzoxazine (1.8 g, 3.86 mmol) in dioxane (10 mL) and water (1 mL) was added [2-[[tert-butyl(dimethyl)silyl]oxymethyl]thieno[3,2-b]pyridin-7-yl]boronic acid (1.9 g, 5.80 mmol), potassium carbonate (1.3 g, 9.66 mmol) and dichloro[1,1′-bis(diphenylphosphino)ferrocene]palladium(II) (565 mg, 0.77 mmol). After stirred at 110° C. for 3 h, the reaction mixture was filtered and the filtrate was concentrated under reduced pressure. The residue was purified by column chromatography to afford the title compound. LCMS R_{T}=1.568 min, m/z=664.4 [M+H]⁺.

### Step 6: 5-(2-(((tert-butyldimethylsilyl)oxy)methyl)thieno[3,2-b]pyridin-7-yl)-4-(1-(tert-butylsulfonyl)-5,5-dimethylpyrrolidin-3-yl)-3,4-dihydro-2H-benzo[b][1,4]oxazine-7-carbonitrile

To a solution of tert-butyl-[[7-[4-(1-tert-butylsulfonyl-5,5-dimethyl-pyrrolidin-3-yl)-7-chloro-2,3-dihydro-1,4-benzoxazin-5-yl]thieno[3,2-b]pyridin-2-yl]methoxy]-dimethyl-silane (1.4 g, 2.11 mmol) in dioxane (10 mL) were added zinc cyanide (322 mg, 2.74 mmol), N-ethyl-N-isopropylpropan-2-amine (681 mg, 5.27 mmol) and bis(tri-tert-butylphosphine) palladium(0) (215 mg, 0.42 mmol). After stirred at 90° C. for 20 h, the reaction mixture was diluted with water (50 mL) and extracted with dichloromethane (20 mL×3). The combined organic layers were washed with brine (25 mL), dried over anhydrous sodium sulfate, filtered and the filtrate was concentrated under reduced pressure. The residue was purified by column chromatography to afford the title compound. LCMS R_{T}=0.645 min, m/z=655.7 [M+H]⁺.

### Step 7: (S)-4-(1-(tert-butylsulfonyl)-5,5-dimethylpyrrolidin-3-yl)-5-(2-(hydroxymethyl)thieno[3,2-b]pyridin-7-yl)-3,4-dihydro-2H-benzo[b][1,4]oxazine-7-carbonitrile and (R)-4-(1-(tert-butylsulfonyl)-5,5-dimethylpyrrolidin-3-yl)-5-(2-(hydroxymethyl)thieno[3,2-b]pyridin-7-yl)-3,4-dihydro-2H-benzo[b][1,4]oxazine-7-carbonitrile

To a solution of 5-[2-[[tert-butyl(dimethyl)silyl]oxymethyl]thieno[3,2-b]pyridin-7-yl]-4-(1-tert-butylsulfonyl-5,5-dimethyl-pyrrolidin-3-yl)-2,3-dihydro-1,4-benzoxazine-7-carbonitrile (470 mg, 0.72 mmol) in THF (10 mL) was added tetrabutylammonium fluoride (0.97 mL, 1 M in THF) at 25° C. After stirred at 25° C. for 2 h, the reaction mixture was diluted with water (20 mL) and extracted with dichloromethane (15 mL×3). The combined organic layers were washed with brine (25 mL), dried over anhydrous sodium sulfate, filtered and the filtrate was concentrated under reduced pressure. The residue was purified by column. The racemate was separated by SFC to give first eluting fraction 133-i (Rt=1.575 min; LCMS R_{T}=0.480 min, m/z=541.3 [M+H]+) and second eluting fraction 133-ii (Rt=1.989 min; LCMS R_{T}=0.480 min, m/z=541.3 [M+H]+).

### Step 8

Example 133a was prepared from 133-i and 1-(2,2,2-trifluoroethyl)pyrimidine-2,4-dione (32 mg, 0.17 mmol), following the procedure described in the synthesis of Example 123a. The product was purified by preparative HPLC. ¹H NMR (400 MHz, CD₃OD) δ=8.80-8.71 (m, 1H), 7.71-7.45 (m, 3H), 7.27 (dd, J=2.0, 8.8 Hz, 2H), 5.87 (d, J=8.0 Hz, 1H), 5.40 (s, 2H), 4.62 (d, J=8.8 Hz, 2H), 4.38-4.10 (m, 2H), 3.89-3.70 (m, 1H), 3.44-3.32 (m, 2H), 2.69-2.50 (m, 1H), 2.64 (br s, 1H), 1.86-1.38 (m, 2H), 1.31-1.15 (m, 3H), 0.75-0.53 (m, 3H). LCMS R_{T}=0.468 min, m/z=597.2 [M+H]+

Example 133b was prepared from 133-ii and 1-(2,2,2-trifluoroethyl)pyrimidine-2,4-dione (32 mg, 0.17 mmol), following the procedure described in the synthesis of Example 123a. The product was purified by preparative HPLC. H NMR (400 MHz, CD₃OD) δ=9.00-8.66 (m, 1H), 8.49 (s, 1H), 7.80-7.45 (m, 3H), 7.30 (dd, J=2.0, 10.8 Hz, 2H), 5.89 (d, J=8.0 Hz, 1H), 5.42 (s, 2H), 4.65 (q, J=8.8 Hz, 2H), 4.42-4.01 (m, 2H), 3.92-3.73 (m, 1H), 3.51-3.34 (m, 2H), 3.15-2.46 (m, 2H), 1.96-1.49 (m, 2H), 1.38-1.16 (m, 3H), 0.84-0.60 (m, 3H). LCMS R_{T}=1.008 min, m/z=597.3 [M+H]+

The absolute configuration was tentatively assigned based on the relative potency of the pair diastereomers in biology assays.

### Example 134: (S)-4-(5,5-dimethylpyrrolidin-3-yl)-5-(2-((4-methyl-6-oxopyridazin-1(6H)-yl)methyl)thieno[3,2-b]pyridin-7-yl)-3,4-dihydro-2H-benzo[b][1,4]oxazine-7-carbonitrile, formic acid salt

Prepared from 133-i and 5-methylpyridazin-3(2H)-one, following the procedure described in the synthesis of Example 123a. The product was purified by preparative HPLC. ¹H NMR (400 MHz, CD30D) 6=8.97-8.69 (m, 1H), 8.53 (s, 1H), 7.89 (d, J=2.0 Hz, 1H), 7.71-7.42 (m, 2H), 7.29 (dd, J=2.0, 9.2 Hz, 2H), 6.82 (s, 1H), 5.63 (d, J=5.2 Hz, 2H), 4.42-4.07 (m, 2H), 3.97-3.70 (m, 1H), 3.47-3.37 (m, 1H), 3.06-2.52 (m, 2H), 2.27 (s, 3H), 1.87-1.01 (m, 6H), 0.86-0.54 (m, 3H). LCMS R_{T}=1.044 min, m/z=513.4 [M+H]⁺

### Example 135: (S)-5-(2-((4-cyclopropyl-6-oxopyridazin-1(6H)-yl)methyl)thieno[3,2-b]pyridin-7-yl)-4-(5,5-dimethylpyrrolidin-3-yl)-3,4-dihydro-2H-benzo[b][1,4]oxazine-7-carbonitrile

Prepared from 135-i and 5-cyclopropylpyridazin-3(2H)-one, following the procedure described in the synthesis of Example 123a. The product was purified by preparative HPLC. ¹H NMR (400 MHz, CD₃OD) δ=8.79-8.72 (m, 1H), 8.51-8.46 (m, 1H), 7.81-7.77 (m, 1H), 7.64-7.44 (m, 2H), 7.32-7.22 (m, 2H), 6.62-6.57 (m, 1H), 5.64-5.54 (m, 2H), 4.16 (s, 2H), 3.92-3.69 (m, 1H), 3.35 (s, 1H), 3.16-2.49 (m, 2H), 1.92-1.69 (m, 2H), 1.67-1.40 (m, 1H), 1.35-1.21 (m, 3H), 1.20-1.14 (m, 2H), 0.93-0.87 (m, 2H), 0.84-0.46 (m, 4H). LCMS: R_{T}=0.440 min, m/z=539.3 [M+H]⁺.

### Example 136: (S)-5-(2-((4-(tert-butyl)-6-oxopyridazin-1(6H)-yl)methyl)thieno[3,2-b]pyridin-7-yl)-4-(5,5-dimethylpyrrolidin-3-yl)-3,4-dihydro-2H-benzo[b][1,4]oxazine-7-carbonitrile, formic acid salt

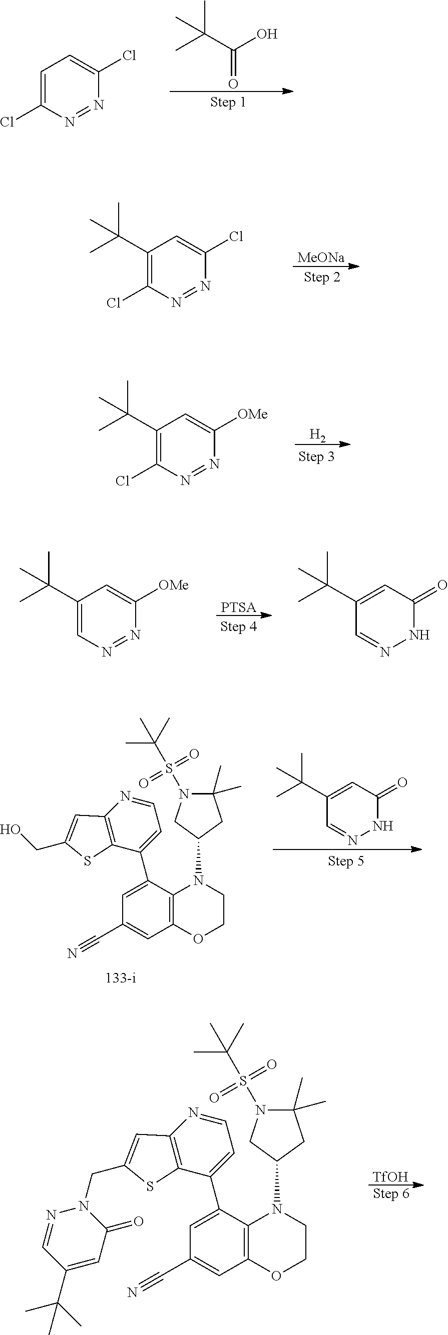

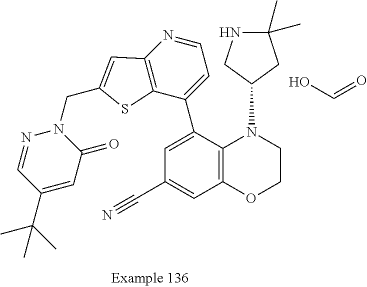

### Step 1: 4-(tert-butyl)-3,6-dichloropyridazine

To a solution of trimethylacetic acid (960 mg, 9.40 mmol) in water (10 mL) was added 3,6-dichloropyridazine (1.0 g, 6.71 mmol), silver nitrate (0.4 g, 2.47 mmol) in water (1 mL), trifluoroacetic acid (153 mg, 1.34 mmol) and ammonium persulfate (2.5 g, 10.74 mmol) in water at 55° C. After stirred at 75° C. for 1 h under nitrogen atmosphere, the reaction mixture was quenched by addition of saturated sodium bicarbonate (10 mL) and extracted with dichloromethane (10 mL×2). The combined organic layers were washed with brine (20 mL), dried over anhydrous sodium sulfate, filtered and the filtrate was concentrated under reduced pressure. The residue was purified by column chromatography to afford the title compound. ¹H NMR (400 MHz, CDCl₃) δ=7.53-7.41 (m, 1H), 1.53-1.47 (m, 9H). LCMS R_{T}=1.948 min, m/z=205.2 [M+H]⁺.

### Step 2: 4-(tert-butyl)-3-chloro-6-methoxypyridazine

To a solution of 4-(tert-butyl)-3,6-dichloropyridazine (900 mg, 4.39 mmoL) in methyl alcohol (15 mL) was added sodium methoxide (1.2 g, 21.94 mmol). After stirred at 20° C. for 1 h under nitrogen atmosphere, the reaction mixture was concentrated under reduced pressure to remove solvent and extracted with dichloromethane (20 mL×2). The combined organic layers were washed with brine (20 mL), dried over anhydrous sodium sulfate, filtered and the filtrate was concentrated under reduced pressure to afford the title compound which was used in the next step without further purification. ¹H NMR (400 MHz, CDCl₃) δ=6.98-6.92 (m, 1H), 4.13-4.10 (m, 3H), 1.49-1.45 (m, 9H). LCMS R_{T}=1.483 min, m/z=201.2 [M+H]⁺.

### Step 3: 5-(tert-butyl)-3-methoxypyridazine

To a solution of 4-(tert-butyl)-3-chloro-6-methoxypyridazine (830 mg, 4.14 mmol) in ethyl alcohol (10 mL) was added 10% palladium on carbon (100 mg) and sodium acetate (679 mg, 8.27 mmol). After stirred at 20° C. for 1 h under hydrogen atmosphere, the reaction mixture was concentrated under reduced pressure to remove solvent and extracted with ethyl acetate (20 mL×2). The combined organic layers were washed with brine (20 mL), dried over anhydrous sodium sulfate, filtered and the filtrate was concentrated under reduced pressure to afford the title compound which was used in the next step without further purification. ¹H NMR (400 MHz, CDCl₃) δ=8.97-8.86 (m, 1H), 6.91-6.82 (m, 1H), 4.16-4.09 (m, 3H), 1.35-1.32 (m, 9H). LCMS R_{T}=1.087 min, m/z=167.3 [M+H]⁺.

### Step 4: 5-(tert-butyl)pyridazin-3(2H)-one

To a solution of 5-(tert-butyl)-3-methoxypyridazine (730 mg, 4.39 mmol) in N,N-dimethylacetamide (10 mL) was added methylbenzenesulfonic acid (3.8 g, 21.96 mmol) and lithium chloride (931 mg, 21.96 mmol). After stirred at 120° C. for 0.5 h under nitrogen atmosphere, the reaction mixture was diluted with water (20 mL) and extracted with ethyl acetate (20 mL×2). The combined organic layers were washed with brine (20 mL), dried over anhydrous sodium sulfate, filtered and the filtrate was concentrated under reduced pressure. The residue was purified by preparative HPLC to afford the title compound. ¹H NMR (400 MHz, CDCl₃) δ=11.68-11.13 (m, 1H), 7.95-7.79 (m, 1H), 6.89-6.71 (m, 1H), 1.37-1.26 (m, 9H). LCMS R_{T}=1.591 min, m/z=153.1 [M+H]⁺.

### Step 5: (S)-5-(2-((4-(tert-butyl)-6-oxopyridazin-1(6H)-yl)methyl)thieno[3,2-b]pyridin-7-yl)-4-(1-(tert-butylsulfonyl)-5,5-dimethylpyrrolidin-3-yl)-3,4-dihydro-2H-benzo[b][1,4]oxazine-7-carbonitrile

To a solution of (S)-4-(1-(tert-butylsulfonyl)-5,5-dimethylpyrrolidin-3-yl)-5-(2-(hydroxymethyl)thieno[3,2-b]pyridin-7-yl)-3,4-dihydro-2H-benzo[b][1,4]oxazine-7-carbonitrile (27 mg, 0.05 mmol) in toluene (1 mL) was added 5-(tert-butyl)pyridazin-3(2H)-one (11 mg, 0.07 mmol), tetramethylazodicarboxamide (17 mg, 0.10 mmol) and tributylphosphane (40 mg, 0.20 mmol). After stirred at 95° C. for 1 h under nitrogen atmosphere, the reaction mixture was filtered and the filtrate was concentrated under reduced pressure. The residue was purified by preparative TLC to afford the title compound. LCMS R_{T}=1.817 min, m/z=675.3 [M+H]⁺.

### Step 6

To a solution of (S)-5-(2-((4-(tert-butyl)-6-oxopyridazin-1(6H)-yl)methyl)thieno[3,2-b]pyridin-7-yl)-4-(1-(tert-butylsulfonyl)-5,5-dimethylpyrrolidin-3-yl)-3,4-dihydro-2H-benzo[b][1,4]oxazine-7-carbonitrile (26 mg, 0.04 mmol) in dichloromethane (1 mL) was added trifluoromethanesulfonic acid (52 mg, 0.64 mmol). After stirred at 20° C. for 0.5 h, the reaction mixture was concentrated under reduced pressure. The residue was purified by preparative HPLC to afford Example 136. ¹H NMR (400 MHz, CD₃OD) δ=8.85-8.68 (m, 1H), 8.60-8.46 (m, 1H), 8.22-8.08 (m, 1H), 7.70-7.40 (m, 4H), 7.33-7.20 (m, 1H), 6.88-6.76 (m, 1H), 5.72-5.49 (m, 2H), 4.39-3.66 (m, 3H), 3.16-2.27 (m, 2H), 1.99-1.04 (m, 15H), 0.94-0.34 (m, 4H). LCMS R_{T}=1.287 min, m/z=555.5 [M+H]⁺.

### Example 137: 1-[[7-[1-(2-azaspiro[3.3]heptan-6-yl)-6-chloro-3,4-dihydro-2H-quinolin-8-yl]thieno[3,2-b]pyridin-2-yl]methyl]pyrrolidine-2,5-dione, formic acid salt

Prepared from 6-chloro-1,2,3,4-tetrahydroquinoline and tert-butyl 6-oxo-2-azaspiro[3.3]heptane-2-carboxylate following the procedure described in the synthesis of Example 1. The final compound was purified by RP-HPLC (2-32% acetonitrile in water and 0.225% formic acid). ¹H NMR (400 MHz, CD₃OD) δ=8.68 (d, J=4.8 Hz, 1H), 8.46 (s, 1H), 7.44 (s, 1H), 7.40 (d, J=4.8 Hz, 1H), 7.12-7.04 (m, 2H), 4.92 (s, 2H), 3.76 (s, 2H), 3.48 (s, 2H), 3.20 (s, 2H), 3.16-3.00 (m, 1H), 2.84 (t, J=6.4 Hz, 2H), 2.76 (s, 4H), 2.04 (s, 2H), 1.80 (s, 2H), 1.60-0.74 (m, 2H). LCMS R_{T}=1.581 min, m/z=507.3 [M+H]⁺.

### Example 138: 1-[[7-[6-chloro-1-(2-methyl-2-azaspiro[3.3]heptan-6-yl)-3,4-dihydro-2H-quinolin-8-yl]thieno[3,2-b]pyridin-2-yl]methyl]pyrrolidine-2,5-dione, hydrochloride acid salt

To a solution of 1-[[7-[1-(2-azaspiro[3.3]heptan-6-yl)-6-chloro-3,4-dihydro-2H-quinolin-8-yl]thieno[3,2-b]pyridin-2-yl]methyl]pyrrolidine-2,5-dione (Example 137; 220 mg, 0.43 mmol) in acetonitrile (3 mL) was added formaldehyde (86 mg, 0.87 mmol), sodium ttriacetoxyborohydride (276 mg, 1.30 mmol) and N,N-diethylethanamine (132 mg, 1.30 mmol) at 20° C. After stirring at 20° C. for 60 min, the reaction was concentrated under reduced pressure. The residue was purified by RP-HPLC (2-32% acetonitrile in water and 0.225% formic acid) to afford the title compound. ¹H NMR (400 MHz, CD₃OD) δ=8.98 (d, J=6.4 Hz, 1H), 8.04 (d, J=6.4 Hz, 1H), 7.76 (s, 1H), 7.36-7.18 (m, 2H), 5.06 (s, 2H), 4.16 (s, 1H), 4.10-4.00 (m, 1H), 3.92-3.76 (m, 2H), 3.70 (d, J=10.4 Hz, 1H), 3.32-3.12 (m, 2H), 3.04 (s, 1H), 2.90 (t, J=6.4 Hz, 2H), 2.80 (s, 3H), 2.72 (s, 3H), 2.40-1.98 (m, 2H), 1.88 (s, 2H), 1.80-0.72 (m, 2H). LCMS R_{T}=1.693 min, m/z=521.1 [M+H]⁺

### Example 139a and 139b: (S)-1-((7-(6-chloro-1-(5-azaspiro[3.4]octan-7-yl)-1,2,3,4-tetrahydroquinolin-8-yl)thieno[3,2-b]pyridin-2-yl)methyl)pyrrolidine-2,5-dione and (R)-1-((7-(6-chloro-1-(5-azaspiro[3.4]octan-7-yl)-1,2,3,4-tetrahydroquinolin-8-yl)thieno[3,2-b]pyridin-2-yl)methyl)pyrrolidine-2,5-dione

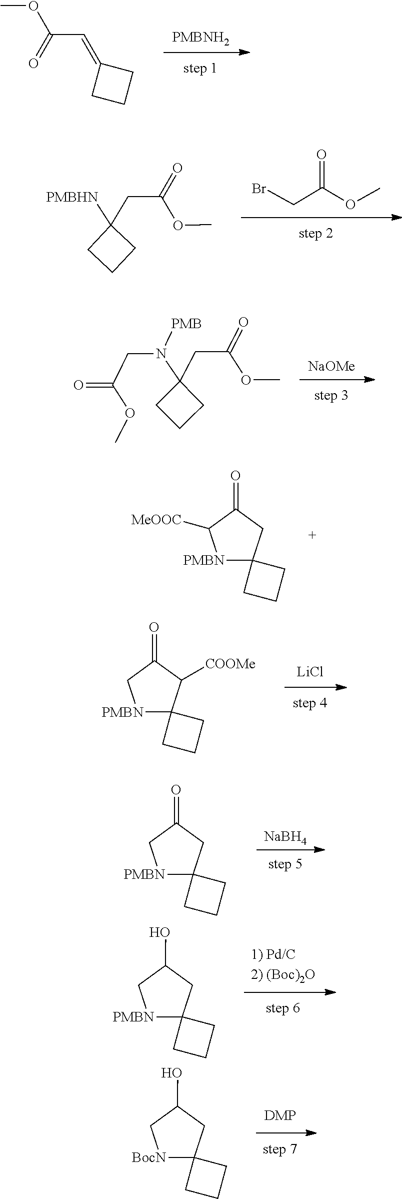

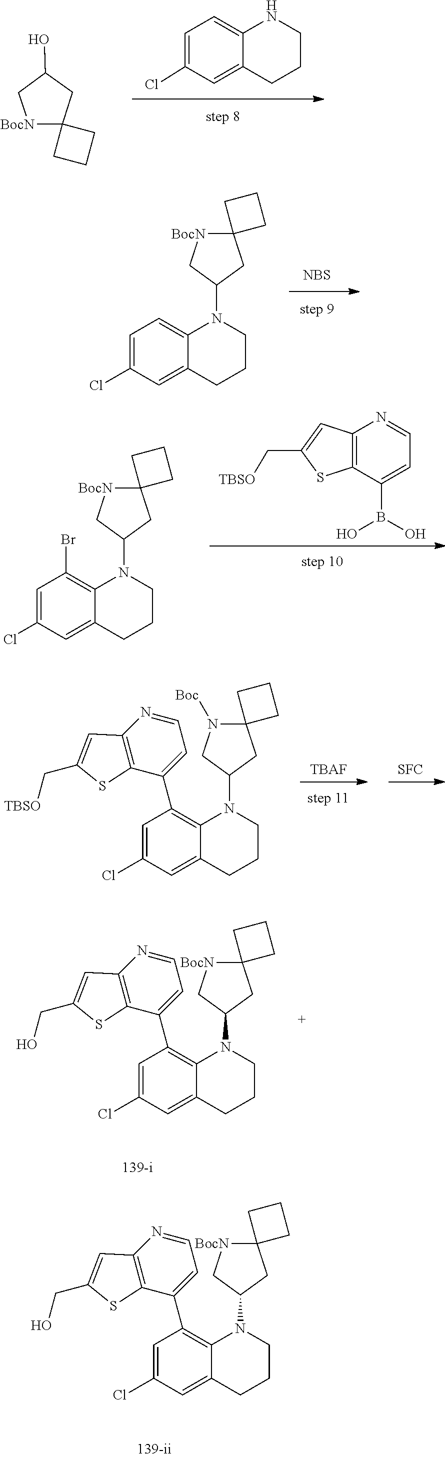

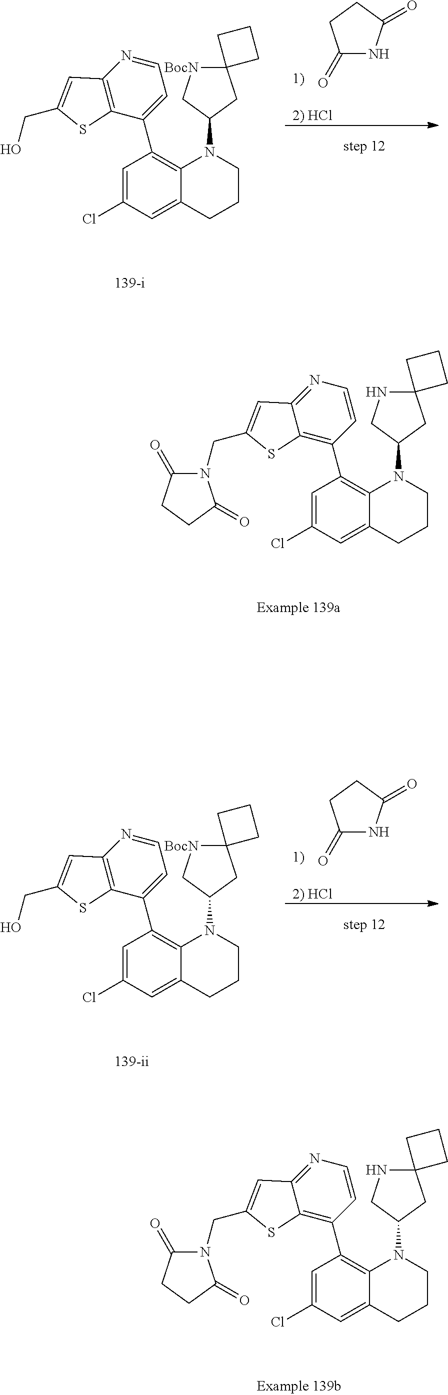 [SMILES: CC(C)(C)OC(=O)N1C[C@H](N2CCCc3cc(Cl)cc(-c4ccnc5cc(CO)sc45)c32)CC12CCC2 ; O=C1CCC(=O)N1 ; O=C1CCC(=O)N1Cc1cc2nccc(-c3cc(Cl)cc4c3N([C@H]3CNC5(CCC5)C3)CCC4)c2s1 ; CC(C)(C)OC(=O)N1C[C@@H](N2CCCc3cc(Cl)cc(-c4ccnc5cc(CO)sc45)c32)CC12CCC2 ; O=C1CCC(=O)N1 ; O=C1CCC(=O)N1Cc1cc2nccc(-c3cc(Cl)cc4c3N([C@@H]3CNC5(CCC5)C3)CCC4)c2s1]

### Step 1: methyl 2-(1-((4-methoxybenzyl)amino)cyclobutyl)acetate

To a solution of methyl 2-cyclobutylideneacetate (15 g, 118.90 mmol) in ethyl alcohol (100 mL) was added (4-methoxyphenyl)methanamine (16.31 g, 118.90 mmol). After stirred 80° C. for 12 h, the reaction mixture was quenched by saturated sodium bicarbonate (40 mL), then the reaction mixture was diluted with water (10 mL), extracted with ethyl acetate (30 mL×2). The combined organic layers were washed with brine (60 mL), dried over anhydrous sodium sulfate and concentrated under reduced pressure to afford the title compound. LCMS R_{T}=0.638 min, m/z=264.2 [M+H]⁺.

### Step 2: methyl 2-(1-((2-methoxy-2-oxoethyl)(4-methoxybenzyl)amino)cyclobutyl)acetate

To a solution of methyl 2-(1-((4-methoxybenzyl)amino)cyclobutyl)acetate (75 g, 284.81 mmol) and sodium bicarbonate (47.85 g, 569.62 mmol) in acetonitrile (400 mL) was added methyl 2-bromoacetate (87.14 g, 569.62 mmol) at 20° C. After stirred at 50° C. for 12 h, the reaction mixture was quenched by water (30 mL), then the reaction mixture was diluted with water (10 mL), extracted with ethyl acetate (20 mL×3). The combined organic layers were washed with brine (60 mL), dried over anhydrous sodium sulfate and concentrated under reduced pressure to give a residue. The residue was purified by column chromatography to afford the title compound. LCMS R_{T}=0.779 min, m/z=336.2 [M+H]⁺.

### Step 3: methyl 5-(4-methoxybenzyl)-7-oxo-5-azaspiro[3.4]octane-6-carboxylate and methyl 5-(4-methoxybenzyl)-7-oxo-5-azaspiro[3.4]octane-8-carboxylate

To a solution of methyl 2-(1-((2-methoxy-2-oxoethyl)(4-methoxybenzyl)amino)cyclobutyl)acetate (30 g, 89.45 mmol) in methyl alcohol (200 mL) was added sodium methoxide (24.16 g, 447.24 mmol) at 20° C. After stirred at 60° C. for 12 h, the reaction mixture was quenched by water (30 mL), then the reaction mixture was diluted with water (10 mL), extracted with ethyl acetate (20 mL×3). The combined organic layers were washed with brine (60 mL), dried over anhydrous sodium sulfate and concentrated under reduced pressure to afford the title compound which was used in next step without further purification. LCMS R_{T}=0.757 min, m/z=304.2 [M+H]⁺.

### Step 4: 5-(4-methoxybenzyl)-5-azaspiro[3.4]octan-7-one

The mixture methyl 5-(4-methoxybenzyl)-7-oxo-5-azaspiro[3.4]octane-6-carboxylate with methyl 5-(4-methoxybenzyl)-7-oxo-5-azaspiro[3.4]octane-8-carboxylate (5 g, 16.48 mmol) was dissolved in dimethyl sulfoxide (5 mL) and water (0.1 mL). After stirred at 130° C. for 0.5 h, the reaction mixture was quenched by water (100 mL), extracted with ethyl acetate (50 mL×3). The combined organic layers were washed with brine (60 mL), dried over anhydrous sodium sulfate and concentrated under reduced pressure. The residue was purified by preparative HPLC to afford the title compound. LCMS R_{T}=0.647 min, m/z=246.3 [M+H]⁺.

### Step 5: 5-(4-methoxybenzyl)-5-azaspiro[3.4]octan-7-ol

To a solution of 5-(4-methoxybenzyl)-5-azaspiro[3.4]octan-7-one (2 g, 8.15 mmol) in methyl alcohol (10 mL) was added sodium borohydride (1.01 g, 26.79 mmol). After stirred at 25° C. for 16 h, the reaction mixture was concentrated under reduced pressure to give a residue. The residue was purified by preparative HPLC to afford the title compound Step 6: tert-butyl 7-hydroxy-5-azaspiro[3.4]octane-5-carboxylate

To a solution of 5-(4-methoxybenzyl)-5-azaspiro[3.4]octan-7-ol (2 g, 8.09 mmol) in methyl alcohol (30 mL) was added 5% palladium carbon (200 mg, 0.81 mmol) at 20° C. After stirred 20° C. for 2 h under hydrogen atmosphere, the reaction mixture was filtered and the filtrate was concentrated under reduced pressure. The residue was dissolved in dichloromethane (10 mL), then triethylamine (1.19 g, 11.79 mmol) and di-tert-butyl dicarbonate (2.06 g, 9.44 mmol) was added. After stirred at 20° C. for 1 h, the reaction mixture was concentrated under reduced pressure. The residue was purified by column chromatography to afford the title compound. LCMS R_{T}=0.860 min, m/z=172.2 [M+H−56]⁺.

### Step 7: tert-butyl 7-oxo-5-azaspiro[3.4]octane-5-carboxylate

To a solution of tert-butyl 7-hydroxy-5-azaspiro[3.4]octane-5-carboxylate (500 mg, 2.20 mmol) in dichloromethane (10 mL) was added Dess-Martin (2.80 g, 6.60 mmol) at 20° C. After stirred 20° C. for 1 h, the reaction mixture was concentrated under reduced pressure. The residue was purified by column chromatography to afford the title compound.

### Step 8: tert-butyl 7-(6-chloro-3,4-dihydroquinolin-1(2H)-yl)-5-azaspiro[3.4]octane-5-carboxylate

To a solution of tert-butyl 7-oxo-5-azaspiro[3.4]octane-5-carboxylate (400 mg, 1.78 mmol) and 6-chloro-1,2,3,4-tetrahydroquinoline (446 mg, 2.66 mmol) in acetic acid (3 mL) was added sodium borohydride acetate (752 mg, 3.55 mmol) at 20° C. After stirred at 20° C. for 1 h, the reaction mixture was concentrated under reduced pressure. The residue was purified by preparative HPLC to afford the title compound. LCMS R_{T}=1.223 min, m/z=377.1 [M+H]⁺.

### Step 9: tert-butyl 7-(8-bromo-6-chloro-3,4-dihydroquinolin-1(2H)-yl)-5-azaspiro[3.4]octane-5-carboxylate

To a solution of tert-butyl 7-(6-chloro-3,4-dihydroquinolin-1(2H)-yl)-5-azaspiro[3.4]octane-5-carboxylate (100 mg, 0.27 mmol) in dichloromethane (3 mL) was added N-bromosuccinimide (52 mg, 0.29 mmol) at 0° C. After stirred at 0° C. for 30 min, the reaction mixture was concentrated under reduced pressure. The residue was purified by pre-TLC to afford the title compound. LCMS R_{T}=1.283 min, m/z=456.9 [M+2+H]⁺.

### Step 10: tert-butyl 7-(8-(2-(((tert-butyldimethylsilyl)oxy)methyl)thieno[3,2-b]pyridin-7-yl)-6-chloro-3,4-dihydroquinolin-1(2H)-yl)-5-azaspiro[3.4]octane-5-carboxylate

To a solution of tert-butyl 7-(8-bromo-6-chloro-3,4-dihydroquinolin-1(2H)-yl)-5-azaspiro[3.4]octane-5-carboxylate (100 mg, 0.22 mmol), (2-(((tert-butyldimethylsilyl)oxy)methyl)thieno[3,2-b]pyridin-7-yl)boronic acid (106 mg, 0.33 mmol) and cesium carbonate (214 mg, 0.66 mmol) in dioxane (5 mL) and water (0.5 mL) was added dichloro[1,1′-bis(diphenylphosphino)ferrocene]palladium(II) (32 mg, 0.04 mmol) at 20° C. After stirred at 100° C. for 3 h, the reaction mixture was concentrated under reduced pressure. The residue was purified by pre-TLC to afford the title compound. LCMS R_{T}=1.221 min, m/z=654.2 [M+H]⁺.

### Step 11: (R)-tert-butyl 7-(6-chloro-8-(2-(hydroxymethyl)thieno[3,2-b]pyridin-7-yl)-3,4-dihydroquinolin-1(2H)-yl)-5-azaspiro[3.4]octane-5-carboxylate (S)-tert-butyl 7-(6-chloro-8-(2-(hydroxymethyl)thieno[3,2-b]pyridin-7-yl)-3,4-dihydroquinolin-1(2H)-yl)-5-azaspiro[3.4]octane-5-carboxylate

To a solution of tert-butyl 7-(8-(2-(((tert-butyldimethylsilyl)oxy)methyl)thieno[3,2-b]pyridin-7-yl)-6-chloro-3,4-dihydroquinolin-1(2H)-yl)-5-azaspiro[3.4]octane-5-carboxylate (120 mg, 0.18 mmol) in THF (1 mL) was added tetrabutylammonium fluoride in THF (1M, 0.1 mL) at 20° C. After stirred at 20° C. for 10 min, the reaction mixture was concentrated under reduced pressure. The residue was purified by pre-TLC. The racemic product was separated by SFC to give first eluting fraction (139-i, R₁=2.967 min, LCMS R_{T}=0.730 min, m/z=540.2 [M+H]+) and second eluting fraction (139-ii, Rₜ=3.541 min, LCMS R_{T}=0.730 min, m/z=540.2 [M+H]+).

### Step 12

To a solution of 139-i (25 mg, 0.05 mmol) and succinimide (9 mg, 0.09 mmol) and triphenylphosphine (24 mg, 0.09 mmol) in THF (1 mL) was added diisopropyl azodicarboxylate (18 mg, 0.09 mmol) at 0° C. After stirred at 50° C. for 10 min, the reaction mixture was concentrated under reduced pressure. The residue was dissolved in hydrochloric acid in dioxane (4 M, 2 mL). After stirred at 20° C. for 10 min, the reaction mixture was concentrated under reduced pressure. The residue was purified by preparative HPLC to afford Example 139a. ¹H NMR (400 MHz, CD₃OD) δ=8.68 (d, J=4.8 Hz, 1H), 7.52 (s, 1H), 7.44 (d, J=4.8 Hz, 1H), 7.16-7.04 (m, 2H), 4.96 (s, 2H), 3.52-3.40 (m, 1H), 3.20 (d, J=16.4 Hz, 2H), 2.92-2.80 (m, 2H), 2.76 (s, 5H), 2.08-1.72 (m, 5H), 1.64-1.08 (m, 6H). LCMS R_{T}=1.375 min, m/z=521.1 [M+H]⁺.

Example 139b was prepared from 139-ii and succinimide (9 mg, 0.09 mmol), following the procedure described above. The product was purified by preparative HPLC. ¹H NMR (400 MHz, CD₃OD) δ=8.68 (d, J=4.8 Hz, 1H), 7.52 (s, 1H), 7.44 (d, J=4.8 Hz, 1H), 7.20-7.04 (m, 2H), 4.96 (s, 2H), 3.52-3.40 (m, 1H), 3.20 (d, J=16.4 Hz, 2H), 2.86 (d, J=4.4 Hz, 2H), 2.76 (s, 5H), 2.04-1.72 (m, 5H), 1.64-1.16 (m, 6H). LCMS R_{T}=1.372 min, m/z=521.3 [M+H]⁺.

The absolute configuration was tentatively assigned based on the relative potency of the pair diastereomers in biology assays.

### Example 140: (S)-1-((7-(6-chloro-1-(5-methyl-5-azaspiro[3.4]octan-7-yl)-1,2,3,4-tetrahydroquinolin-8-yl)thieno[3,2-b]pyridin-2-yl)methyl)pyrrolidine-2,5-dione

To a solution of Example 139b (30 mg, 0.06 mmol) in acetonitrile (0.5 mL) was added formaldehyde (9 mg, 0.12 mmol), sodium borohydride acetate (37 mg, 0.17 mmol) and trimethylamine (17 mg, 0.17 mmol). After stirred at 25° C. for 1 h, the reaction mixture was filtered and the filtrate was concentrated under reduced pressure. The residue was purified by preparative HPLC to afford the title compound. ¹H NMR (400 MHz, CD₃OD) δ=8.72-8.64 (m, 1H), 7.54-7.45 (m, 1H), 7.44-7.37 (m, 1H), 7.16-7.06 (m, 2H), 5.01-4.94 (m, 2H), 3.56-3.43 (m, 1H), 3.29-3.09 (m, 2H), 2.90-2.71 (m, 7H), 2.28-2.14 (m, 4H), 2.04-1.83 (m, 4H), 1.73-1.41 (m, 4H), 1.26-0.59 (m, 2H). LCMS R_{T}=1.265 min, m/z=535.4 [M+H]⁺.

### Example 141: (S)-1-((7-(6-chloro-1-(5-(2-hydroxyethyl)-5-azaspiro[3.4]octan-7-yl)-1,2,3,4-tetrahydroquinolin-8-yl)thieno[3,2-b]pyridin-2-yl)methyl)pyrrolidine-2,5-dione, formic acid salt

To a solution of Example 139b (50 mg, 0.10 mmol) in acetonitrile (2 mL) was added 2-[tert-butyl(dimethyl)silyl]oxyacetaldehyde (17 mg, 0.10 mmol), sodium borohydride acetate (61 mg, 0.29 mmol) and triethylamine (29 mg, 0.29 mmol) at 25° C. After stirred at 25° C. for 1 h, the reaction mixture was concentrated under reduced pressure to remove the solvent. The residue was added hydrochloric acid (4 mL, 5 M in dioxane). After stirred at 25° C. for 1 h, the reaction mixture was concentrated under reduced pressure. The residue was purified by preparative HPLC to afford the title compound. ¹H NMR (400 MHz, CD₃OD) δ=8.69 (d, J=4.8 Hz, 1H), 8.44 (s, 1H), 7.50 (s, 1H), 7.42 (d, J=4.8 Hz, 1H), 7.15-7.11 (m, 2H), 4.98-4.92 (m, 1H), 4.96 (s, 3H), 3.62-3.41 (m, 3H), 3.29-3.11 (m, 2H), 3.00-2.81 (m, 4H), 2.75 (s, 4H), 2.50-2.17 (m, 3H), 2.02-1.53 (m, 8H). LCMS R_{T}=1.219 min, m/z=565.4 [M+H]⁺.

### Example 142: (S)-1-((7-(6-chloro-1-(5-(2-fluoroethyl)-5-azaspiro[3.4]octan-7-yl)-1,2,3,4-tetrahydroquinolin-8-yl)thieno[3,2-b]pyridin-2-yl)methyl)pyrrolidine-2,5-dione

To a solution of 1-fluoro-2-iodo-ethane (10 mg, 0.06 mmol) and 1-[[7-[1-[(7S)-5-azaspiro[3.4]octan-7-yl]-6-chloro-3,4-dihydro-2H-quinolin-8-yl]thieno[3,2-b]pyridin-2-yl]methyl]pyrrolidine-2,5-dione (139b, 10 mg, 0.02 mmol) in acetonitrile (1 mL) was added potassium carbonate (8 mg, 0.06 mmol) at 20° C. After stirred for 12 h at 70° C., the mixture was diluted with water (10 mL) and extracted with ethyl acetate (5 mL×4). The combined organic layers were washed with brine (20 mL), dried over anhydrous sodium sulfate, filtered and the filtrate was concentrated under reduced pressure. The residue was purified by preparative HPLC to afford the title compound. ¹H NMR (400 MHz, CD30D) 6=8.69 (d, J=4.8 Hz, 1H), 7.58-7.48 (m, 1H), 7.46-7.37 (m, 1H), 7.12 (d, J=4.4 Hz, 2H), 5.02-4.96 (m, 2H), 4.48-4.17 (m, 2H), 3.53-3.40 (m, 1H), 3.31-3.16 (m, 2H), 3.08-2.76 (m, 4H), 2.76-2.53 (m, 4H), 2.51-2.03 (m, 3H), 2.00-1.75 (m, 4H), 1.74-1.08 (m, 5H). LCMS R_{T}=1.302 min, m/z=567.4 [M+H]⁺.

### Example 143a and 143b: 1-((7-(6-chloro-1-((S)-5-((S)-3,3,3-trifluoro-2-hydroxypropyl)-5-azaspiro[3.4]octan-7-yl)-1,2,3,4-tetrahydroquinolin-8-yl)thieno[3,2-b]pyridin-2-yl)methyl)pyrrolidine-2,5-dione, formic acid salt and 1-((7-(6-chloro-1-((S)-5-((R)-3,3,3-trifluoro-2-hydroxypropyl)-5-azaspiro[3.4]octan-7-yl)-1,2,3,4-tetrahydroquinolin-8-yl)thieno[3,2-b]pyridin-2-yl)methyl)pyrrolidine-2,5-dione

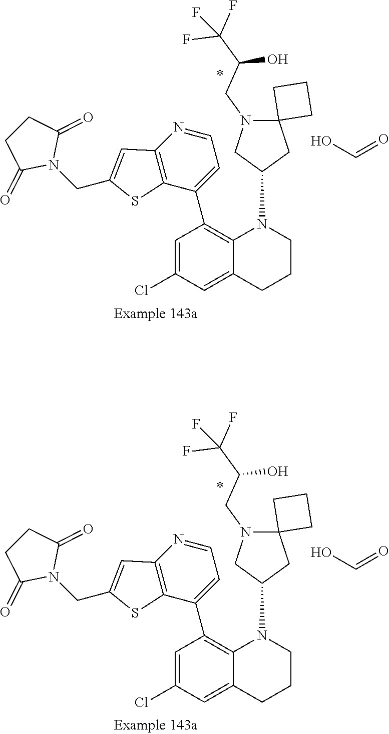

To a solution of 3-bromo-1,1,1-trifluoro-propan-2-ol (74 mg, 0.38 mmol) and 1-[[7-[1-[(7S)-5-azaspiro[3.4]octan-7-yl]-6-chloro-3,4-dihydro-2H-quinolin-8-yl]thieno[3,2-b]pyridin-2-yl]methyl]pyrrolidine-2,5-dione (100 mg, 0.2 mmol) in acetonitrile (1 mL) was added potassium carbonate (53 mg, 0.38 mmol) at 20° C. After stirred for 2 h at 70° C., the mixture was diluted with water (10 mL) and extracted with ethyl acetate (5 mL×4). The combined organic layers were washed with brine (20 mL), dried over anhydrous sodium sulfate, filtered and the filtrate was concentrated under reduced pressure. The mixture was purified by preparative HPLC to give the racemic product. The racemate was separated by SFC and then preparative HPLC to give first eluting fraction (Example 143a, Rt=3.146 min; LCMS R_{T}=1.342 min, m/z=633.4 [M+H]+)

¹H NMR (400 MHz, CD₃OD) δ=8.88-8.55 (m, 1H), 8.20 (s, 1H), 7.59-7.35 (m, 2H), 7.19-6.95 (m, 2H), 4.97 (s, 2H), 3.93-3.80 (m, 1H), 3.56-3.42 (m, 1H), 3.31-3.16 (m, 2H), 3.00-2.69 (m, 8H), 2.24 (br, 1H), 2.17-2.08 (m, 1H), 2.02-1.34 (m, 8H), 1.25-0.25 (m, 2H) and second eluting fraction (Example 143b, Rt=3.318 min; LCMS R_{T}=1.341 min, m/z=633.4 [M+H]+). ¹H NMR (400 MHz, CD30D) 6=8.78-8.52 (m, 1H), 8.21 (s, 1H), 7.76-7.32 (m, 2H), 7.22-7.01 (m, 2H), 4.97 (s, 2H), 3.92-3.66 (m, 1H), 3.54-3.41 (m, 1H), 3.31-3.11 (m, 2H), 2.93-2.58 (m, 8H), 2.34-2.10 (m, 2H), 2.02-1.34 (m, 8H), 1.27-0.55 (m, 2H). (*Absolute configuration was not determined.)

### Example 144: (S)-1-((7-(1-(5-(2-(1H-tetrazol-5-yl)ethyl)-5-azaspiro[3.4]octan-7-yl)-6-chloro-1,2,3,4-tetrahydroquinolin-8-yl)thieno[3,2-b]pyridin-2-yl)methyl)pyrrolidine-2,5-dione

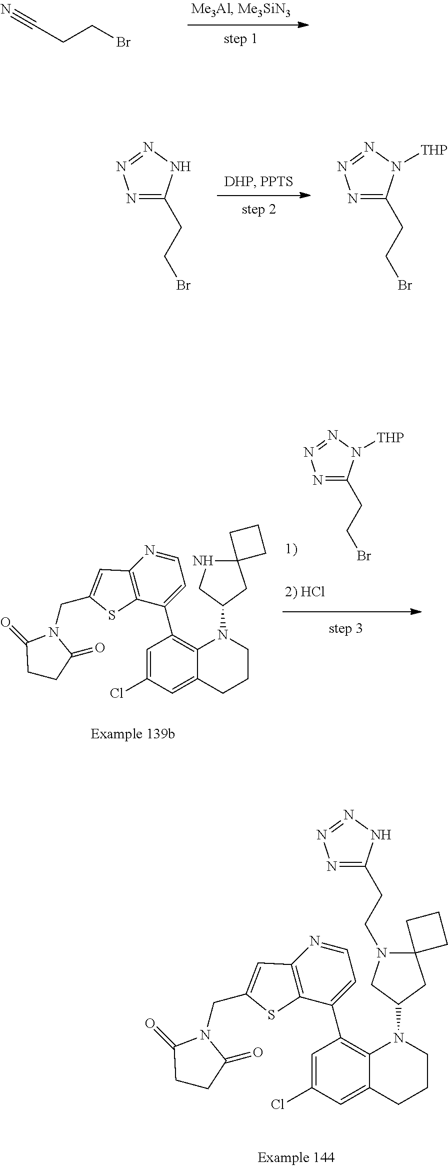

### Step 1: 5-(2-bromoethyl)-1H-tetrazole

To a mixture of trimethylaluminum (2 M in toluene, 9 mL) was added azidotrimethylsilane (17.91 mmol, 2.36 mL) in toluene (5 mL) at 0° C. for 0.5 h. Then to the resulting clear solution was added 3-bromopropanenitrile (2 g, 14.93 mmol, 1.23 mL, 1 eq), again keeping the temperature at 0° C. for 0.5 h, then 20° C. for 0.5 h. After stirred at 80° C. for 12 h, the reaction mixture was adjusted to pH=3 with saturated hydrochloric acid (6 M) and extracted with ethyl acetate (20 mL×3). The combined organic layers were washed with brine (60 mL), dried over anhydrous sodium sulfate, filtered and the filtrate was concentrated under reduced pressure. The residue was purified by preparative HPLC to afford the title compound. ¹H NMR (400 MHz, CDCl₃) δ=3.77-3.68 (m, 2H), 3.64-3.53 (m, 2H)

### Step 2: 5-(2-bromoethyl)-1-(tetrahydro-2H-pyran-2-yl)-1H-tetrazole

To a solution of 5-(2-bromoethyl)-2H-tetrazole (370 mg, 2.09 mmol) and 1,4-dihydropyridine (352 mg, 4.18 mmol) in acetone (5 mL) was added pyridinium p-toluenesulfonate (158 mg, 0.63 mmol) at 20° C. After stirred at 100° C. for 12 h, the reaction mixture was concentrated under reduced pressure. The residue was purified by preparative TLC to afford the title compound. LCMS R_{T}=1.596 min, m/z=701.2 [M+H]⁺

### Step 3

To a solution of 5-(2-bromoethyl)-2-tetrahydropyran-2-yl-tetrazole (75 mg, 0.29 mmol) and potassium iodide (16 mg, 0.1 mmol) in acetonitrile (2 mL) were added potassium carbonate (40 mg, 0.29 mmol) and Example 139b (50 mg, 95.96 mmol) at 20° C. After stirred at 70° C. for 3 h, the reaction mixture was quenched by addition of saturated aqueous ammonium chloride solution (10 mL) and extracted with ethyl acetate (20 mL×2). The combined organic layers were washed with brine (20 mL), dried over anhydrous sodium sulfate, filtered and the filtrate was concentrated under reduced pressure. The residue was dissolved in dioxane (5 ml) and hydrochloric acid (4 M in dioxane, 1.80 mL) was added. After stirred at 25° C. for 0.5 h, the reaction mixture was concentrated under reduced pressure. The residue was purified by preparative HPLC to afford Example 144. ¹H NMR (400 MHz, CD30D) 6=8.63 (d, J=4.8 Hz, 1H), 8.08 (s, 1H), 7.43 (s, 1H), 7.36 (d, J=4.8 Hz, 1H), 7.11-7.01 (m, 2H), 4.89 (s, 2H), 3.58-3.45 (m, 1H), 3.23-2.83 (m, 6H), 2.81-2.47 (m, 7H), 2.39-2.26 (m, 1H), 2.19-1.70 (m, 5H), 1.68-0.55 (m, 5H). LCMS R_{T}=1.268 min, m/z=617.5 [M+H]⁺

### Example 145: 3-((7-(6-chloro-1-((S)-5-azaspiro[3.4]octan-7-yl)-1,2,3,4-tetrahydroquinolin-8-yl)thieno[3,2-b]pyridin-2-yl)methyl)-3-azabicyclo[3.1.0]hexane-2,4-dione, formic acid salt

Example 145 was prepared from (S)-tert-butyl 7-(6-chloro-8-(2-(hydroxymethyl)thieno[3,2-b]pyridin-7-yl)-3,4-dihydroquinolin-1(2H)-yl)-5-azaspiro[3.4]octane-5-carboxylate and 3-azabicyclo[3.1.0]hexane-2,4-dione, following the procedure described in the synthesis of Example 54. The product was purified by preparative HPLC. ¹H NMR (400 MHz, CD₃OD) δ=8.70 (d, J=4.8 Hz, 1H), 8.52 (s, 1H), 7.49-7.38 (m, 2H), 7.15 (m, J=2.4, 12.2 Hz, 2H), 4.81 (s, 2H), 3.62-3.41 (m, 1H), 3.28-3.04 (m, 2H), 2.98-2.72 (m, 3H), 2.58 (m, J=3.6, 8.0 Hz, 3H), 2.22-1.83 (m, 5H), 1.82-1.47 (m, 6H), 1.40 (q, J=3.6 Hz, 1H). LCMS R_{T}=1.447 min, m/z=533.3 [M+H]⁺

### Example 146: (S)-3-((7-(6-chloro-1-(5-azaspiro[3.4]octan-7-yl)-1,2,3,4-tetrahydroquinolin-8-yl)thieno[3,2-b]pyridin-2-yl)methyl)-1-(3,3-difluorocyclobutyl)pyrimidine-2,4(1H,3H)-dione, formic acid salt

Prepared from the tert-butyl (7S)-7-[6-chloro-8-[2-(hydroxymethyl)thieno[3,2-b]pyridin-7-yl]-3,4-dihydro-2H-quinolin-1-yl]-5-azaspiro[3.4]octane-5-carboxylate and 1-(3,3-difluorocyclobutyl)pyrimidine-2,4-dione, following the procedure described in the synthesis of Example 54. The final product was purified by preparative HPLC to afford the title compound. ¹H NMR (400 MHz, CD₃OD) δ=8.68 (d, J=4.8 Hz, 1H), 8.52 (s, 1H), 7.67 (d, J=8.0 Hz, 1H), 7.56 (s, 1H), 7.45-7.36 (m, 1H), 7.19-7.06 (m, 2H), 5.81 (d, J=8.0 Hz, 1H), 5.37 (s, 1H), 4.62 (dd, J=4.4, 7.6 Hz, 2H), 3.62-3.41 (m, 1H), 3.27-3.10 (m, 2H), 3.08-2.97 (m, 4H), 2.90-2.82 (m, 2H), 2.21-2.06 (m, 2H), 2.05-1.81 (m, 4H), 1.67 (s, 5H), 1.36-0.73 (m, 1H). LCMS R_{T}=1.616 min, m/z=624.4 [M+H]⁺

### Example 147: 3-((7-(6-chloro-1-((S)-5-azaspiro[3.4]octan-7-yl)-1,2,3,4-tetrahydroquinolin-8-yl)thieno[3,2-b]pyridin-2-yl)methyl)-6,6-dimethyl-3-azabicyclo[3.1.0]hexane-2,4-dione, formic acid salt

Prepared from (S)-tert-butyl 7-(6-chloro-8-(2-(hydroxymethyl)thieno[3,2-b]pyridin-7-yl)-3,4-dihydroquinolin-1(2H)-yl)-5-azaspiro[3.4]octane-5-carboxylate and 6,6-dimethyl-3-azabicyclo[3.1.0]hexane-2,4-dione, following the procedure described in the synthesis of Example 54. The final product was purified by HPLC to afford the title compound. ¹H NMR (400 MHz, CD₃OD) δ=8.73 (d, J=4.8 Hz, 1H), 8.53 (s, 1H), 7.57-7.42 (m, 2H), 7.26-7.06 (m, 2H), 3.66-3.52 (m, 1H), 3.33-3.09 (m, 3H), 2.94-2.81 (m, 2H), 2.72-2.31 (m, 3H), 2.29-2.19 (m, 1H), 2.17-1.52 (m, 8H), 1.33-1.10 (m, 7H). LCMS R_{T}=1.632 min, m/z=561.4 [M+H]⁺.

### Example 148: (S)-3-((7-(6-chloro-1-(5-azaspiro[3.4]octan-7-yl)-1,2,3,4-tetrahydroquinolin-8-yl)thieno[3,2-b]pyridin-2-yl)methyl)-1-cyclopropylpyrimidine-2,4(1H,3H)-dione

Prepared from tert-butyl (7S)-7-[6-chloro-8-[2-(hydroxymethyl)thieno[3,2-b]pyridin-7-yl]-3,4-dihydro-2H-quinolin-1-yl]-5-azaspiro[3.4]octane-5-carboxylate and 1-cyclopropylpyrimidine-2,4-dione, following the procedure described in the synthesis of Example 54. The final product was purified by preparative HPLC to afford the title compound. ¹H NMR (400 MHz, CD₃OD) δ=8.65 (d, J=5.2 Hz, 1H), 7.61-7.53 (m, 2H), 7.38 (d, J=4.8 Hz, 1H), 7.10 (d, J=2.4 Hz, 2H), 5.72 (d, J=8.0 Hz, 1H), 5.41-5.33 (m, 2H), 3.40 (s, 1H), 3.25-3.07 (m, 3H), 2.91-2.78 (m, 2H), 1.94-1.80 (m, 6H), 1.58-1.26 (m, 5H), 1.07-0.96 (m, 5H). LCMS R_{T}=1.475 min, m/z=574.4 [M+H]⁺.

### Example 149: (S)-3-((7-(6-chloro-1-(5-azaspiro[3.4]octan-7-yl)-1,2,3,4-tetrahydroquinolin-8-yl)thieno[3,2-b]pyridin-2-yl)methyl)-1-(difluoromethyl)pyrimidine-2,4(1H,3H)-dione

Prepared from tert-butyl (7S)-7-[6-chloro-8-[2-(hydroxymethyl)thieno[3,2-b]pyridin-7-yl]-3,4-dihydro-2H-quinolin-1-yl]-5-azaspiro[3.4]octane-5-carboxylate and 1-(difluoromethyl)pyrimidine-2,4-dione, following the procedure described in the synthesis of Example 54. The final product was purified by preparative HPLC to afford the title compound. ¹H NMR (400 MHz, CD₃OD) δ=8.66 (d, J=4.8 Hz, 1H), 7.80 (d, J=8.4 Hz, 1H), 7.75-7.38 (m, 3H), 7.11 (s, 2H), 5.99 (d, J=8.0 Hz, 1H), 5.43-5.32 (m, 2H), 4.60 (s, 1H), 3.48-3.34 (m, 1H), 3.23-3.05 (m, 2H), 2.90-2.77 (m, 2H), 2.63-2.07 (m, 2H), 1.88 (d, J=6.4 Hz, 4H), 1.60-1.24 (m, 5H). LCMS R_{T}=1.561 min, m/z=584.3 [M+H]⁺.

### Example 150: (S)-3-((7-(6-chloro-1-(5-azaspiro[3.4]octan-7-yl)-1,2,3,4-tetrahydroquinolin-8-yl)thieno[3,2-b]pyridin-2-yl)methyl)-1-methylimidazolidine-2,4-dione

Prepared from tert-butyl (7S)-7-[6-chloro-8-[2-(hydroxymethyl)thieno[3,2-b]pyridin-7-yl]-3,4-dihydro-2H-quinolin-1-yl]-5-azaspiro[3.4]octane-5-carboxylate) and 1-methylimidazolidine-2,4-dione, following the procedure described in the synthesis of Example 108. The final product was purified by preparative HPLC to afford the title compound. ¹H NMR (400 MHz, CD₃OD) δ=8.69 (d, J=4.8 Hz, 1H), 7.54-7.37 (m, 2H), 7.18-7.08 (m, 2H), 4.96 (s, 2H), 4.01 (s, 2H), 3.49 (s, 1H), 3.27-3.07 (m, 2H), 3.00-2.82 (m, 5H), 2.20-1.77 (m, 5H), 1.74-0.66 (m, 7H). LCMS R_{T}=1.278 min, m/z=536.3 [M+H]⁺

### Example 151: (S)-3-((7-(6-chloro-1-(5-azaspiro[3.4]octan-7-yl)-1,2,3,4-tetrahydroquinolin-8-yl)thieno[3,2-b]pyridin-2-yl)methyl)oxazolidine-2,4-dione, formic acid salt

Prepared from tert-butyl (7S)-7-[6-chloro-8-[2-(hydroxymethyl)thieno[3,2-b]pyridin-7-yl]-3,4-dihydro-2H-quinolin-1-yl]-5-azaspiro[3.4]octane-5-carboxylate and oxazolidine-2,4-dione, following the procedure described in the synthesis of Example 108. The final product was purified by preparative HPLC to afford the title compound. ¹H NMR (400 MHz, CD₃OD) δ=8.72 (d, J=4.8 Hz, 1H), 8.54 (s, 1H), 7.58 (s, 1H), 7.46 (d, J=4.8 Hz, 1H), 7.21-7.11 (m, 2H), 5.02 (s, 2H), 3.50 (d, J=6.8 Hz, 1H), 3.27-3.08 (m, 2H), 3.00-2.76 (m, 4H), 2.17-1.82 (m, 5H), 1.79-1.32 (m, 5H), 1.30-0.64 (m, 2H). LCMS R_{T}=1.350 min, m/z=523.3 [M+H]⁺

### Example 152: (S)-3-((7-(6-chloro-1-(5-azaspiro[3.4]octan-7-yl)-1,2,3,4-tetrahydroquinolin-8-yl)thieno[3,2-b]pyridin-2-yl)methyl)-5,5-dimethyloxazolidine-2,4-dione

Prepared from tert-butyl (7S)-7-[6-chloro-8-[2-(hydroxymethyl)thieno[3,2-b]pyridin-7-yl]-3,4-dihydro-2H-quinolin-1-yl]-5-azaspiro[3.4]octane-5-carboxylate and 5,5-dimethyloxazolidine-2,4-dione, following the procedure described in the synthesis of Example 108. The final product was purified by preparative HPLC to afford the title compound. ¹H NMR (400 MHz, CD₃OD) δ=8.73 (d, J=4.8 Hz, 1H), 8.52 (s, 1H), 7.55 (s, 1H), 7.47 (d, J=4.8 Hz, 1H), 7.20-7.13 (m, 2H), 5.01 (s, 2H), 3.63-3.48 (m, 1H), 3.28-3.06 (m, 2H), 3.00-2.35 (m, 4H), 2.22-2.10 (m, 1H), 2.09-1.64 (m, 7H), 1.56 (s, 8H). LCMS: R_{T}=1.497 min, m/z=551.4 [M+H]⁺.

### Example 153: (S)-2-((7-(6-chloro-1-(5-azaspiro[3.4]octan-7-yl)-1,2,3,4-tetrahydroquinolin-8-yl)thieno[3,2-b]pyridin-2-yl)methyl)-5-methylpyridazin-3(2H)-one, formic acid salt

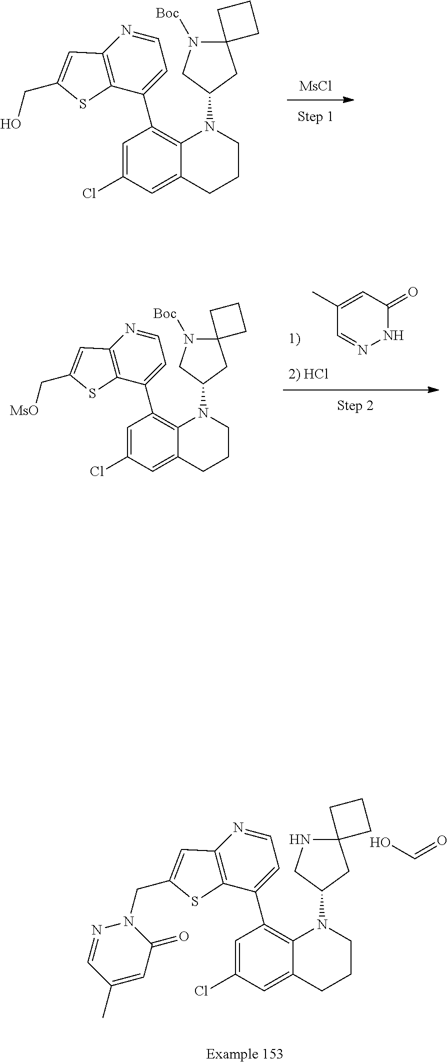

### Step 1: (S)-tert-butyl 7-(6-chloro-8-(2-(((methylsulfonyl)oxy)methyl)thieno[3,2-b]pyridin-7-yl)-3,4-dihydroquinolin-1(2H)-yl)-5-azaspiro[3.4]octane-5-carboxylate

To a solution of (S)-tert-butyl 7-(6-chloro-8-(2-(hydroxymethyl)thieno[3,2-b]pyridin-7-yl)-3,4-dihydroquinolin-1(2H)-yl)-5-azaspiro[3.4]octane-5-carboxylate (65 mg, 0.12 mmol) in dichloromethane (5 mL) was added triethylamine (14 mg, 0.14 mmol) and methane sulfonyl chloride (53 mg, 0.46 mmol) at 0° C. After stirred at 25° C. for 1 h, the reaction mixture was quenched by addition of saturated aqueous sodium bicarbonate (10 mL), and then extracted with dichloromethane (10 mL×3). The combined organic layers were dried over sodium sulphate, filtered and the filtrate was concentrated under reduced pressure to afford the title compound which was used in next step without further purification. LCMS R_{T}=0.641 min, m/z=618.0 [M+H]⁺.

### Step 2

To a mixture of 4-methyl-1H-pyridazin-6-one (7 mg, 0.06 mmol), potassium iodide (7 mg, 0.04 mmol) and potassium carbonate (11 mg, 0.08 mmol) in DMF (3 mL) was added tert-butyl (7S)-7-[6-chloro-8-[2-(methylsulfonyloxymethyl)thieno[3,2-b]pyridin-7-yl]-3,4-dihydro-2H-quinolin-1-yl]-5-azaspiro[3.4]octane-5-carboxylate (25 mg, 0.04 mmol) at 25° C. After stirred at 25° C. for 4 h, the reaction mixture was concentrated under reduced pressure to remove the solvent. The residue was added hydrochloric acid (4 mL, 4 M in dioxane). After stirred at 25° C. for 30 min, the reaction mixture was concentrated under reduced pressure. The residue was purified by preparative HPLC to afford Example 156. ¹H NMR (400 MHz, CD₃OD) δ=8.70 (d, J=4.8 Hz, 1H), 8.53 (s, 1H), 7.87 (d, J=2.0 Hz, 1H), 7.62-7.38 (m, 2H), 7.20-7.11 (m, 2H), 6.80 (s, 1H), 5.61 (s, 2H), 3.62-3.38 (m, 1H), 3.26-3.07 (m, 2H), 2.94-2.82 (m, 2H), 2.25 (d, J=1.2 Hz, 3H), 2.17-0.89 (m, 12H). LCMS R_{T}=1.905 min, m/z=532.1 [M+H]⁺.

### Example 154: (S)-2-((7-(6-chloro-1-(5-azaspiro[3.4]octan-7-yl)-1,2,3,4-tetrahydroquinolin-8-yl)thieno[3,2-b]pyridin-2-yl)methyl)-5-(2,2,2-trifluoroethyl)pyridazin-3(2H)-one, formic acid salt

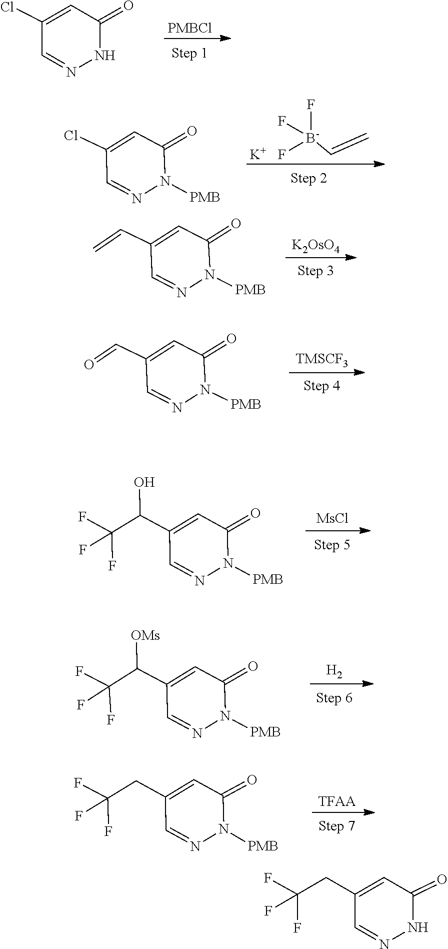

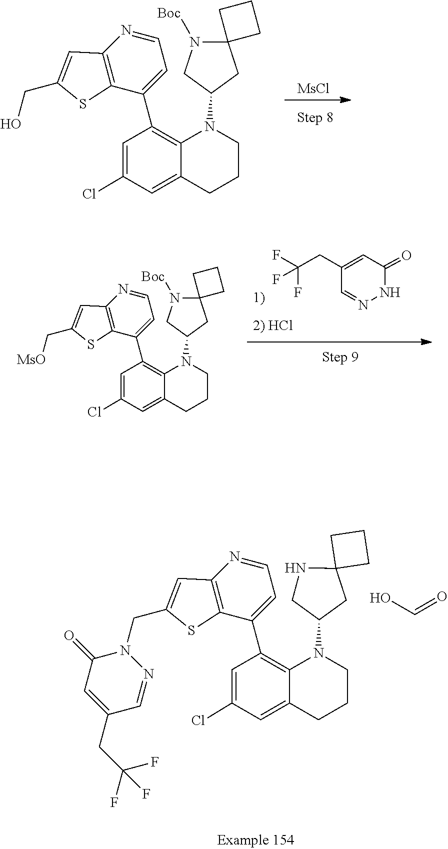

### Step 1: 5-chloro-2-(4-methoxybenzyl)pyridazin-3(2H)-one

To the solution of 5-chloropyridazin-3(2H)-one (5.1 g, 39.22 mmol) in N,N-dimethylacetamide (30 mL) was added potassium carbonate (16.3 g, 117.67 mmol) and 1-(chloromethyl)-4-methoxybenzene (9.2 g, 58.84 mmol). After stirred at 80° C. for 4 h under nitrogen atmosphere, the reaction mixture was extracted with ethyl acetate (30 mL×3). The combined organic layers were washed with brine (30 mL), dried over anhydrous sodium sulfate and concentrated under reduced pressure. The residue was purified by column chromatography to afford the title compound. ¹H NMR (400 MHz, CDCl₃) δ=7.75-7.68 (m, 1H), 7.40-7.35 (m, 2H), 6.99-6.93 (m, 1H), 6.88-6.83 (m, 2H), 5.25-5.20 (m, 2H), 3.79 (s, 3H).

### Step 2: 2-(4-methoxybenzyl)-5-vinylpyridazin-3(2H)-one

A mixture of 5-chloro-2-(4-methoxybenzyl)pyridazin-3(2H)-one (5.1 g, 14.24 mmol), vinyl potassium fluoborate (1.9 g, 14.24 mmol), cesium carbonate (11.6 g, 35.60 mmol) and 4-ditert-butylphosphanyl-N,N-dimethylaniline dichloropalladium (504 mg, 0.71 mmol) in dioxane (36 ml) and water (9 mL) was stirred at 120° C. for 40 min under nitrogen atmosphere in microwave. The reaction mixture was filtered and extracted with ethyl acetate (30 mL×3). The combined organic layers were washed with brine (30 mL), dried over anhydrous sodium sulfate and concentrated under reduced pressure. The residue was purified by column chromatography to afford the title compound. ¹H NMR (400 MHz, CDCl₃) δ=7.93-7.81 (m, 1H), 7.44-7.30 (m, 2H), 6.89-6.80 (m, 2H), 6.78-6.71 (m, 1H), 6.57-6.43 (m, 1H), 5.98-5.86 (m, 1H), 5.64-5.52 (m, 1H), 5.27-5.22 (m, 2H), 3.81-3.75 (m, 3H).

### Step 3: 1-(4-methoxybenzyl)-6-oxo-1,6-dihydropyridazine-4-carbaldehyde

To a solution of 2-(4-methoxybenzyl)-5-vinylpyridazin-3(2H)-one (2.4 g, 9.99 mmol) in water (5 mL) and THF (20 mL) was added sodium periodate (7.8 g, 36.33 mmol) and osmic acid potassium (368 mg, 0.10 mmol). After stirred at 25° C. for 2 h, the reaction mixture was extracted with ethyl acetate (30 mL×3). The combined organic layers were washed with brine (30 mL), dried over anhydrous sodium sulfate and concentrated under reduced pressure. The residue was purified by column chromatography to afford the title compound. ¹H NMR (400 MHz, CDCl₃) δ=10.00-9.92 (m, 1H), 8.20-8.12 (m, 1H), 7.45-7.37 (m, 2H), 7.29-7.27 (m, 1H), 6.91-6.83 (m, 2H), 5.34-5.25 (m, 2H), 3.83-3.76 (m, 3H).

### Step 4: 2-(4-methoxybenzyl)-5-(2,2,2-trifluoro-1-hydroxyethyl)pyridazin-3(2H)-one

To a solution of 1-(4-methoxybenzyl)-6-oxo-1,6-dihydropyridazine-4-carbaldehyde (2.0 g, 7.47 mmol) in THF (10 mL) was added cesium fluoride (908 mg, 5.98 mmol). After cooling to −20° C., the reaction mixture was added trifluoromethyltrimethylsilane (850 mg, 5.98 mmol). After stirred at 25° C. for 16 h under nitrogen atmosphere, the reaction mixture was extracted with ethyl acetate (30 mL×3). The combined organic layers were washed with brine (30 mL), dried over anhydrous sodium sulfate and concentrated under reduced pressure. The residue was purified by preparative HPLC to afford the title compound. ¹H NMR (400 MHz, CD₃OD) δ=8.08-7.96 (m, 1H), 7.36-7.29 (m, 2H), 7.11-7.05 (m, 1H), 6.91-6.85 (m, 2H), 5.28-5.24 (m, 2H), 5.16-5.08 (m, 1H), 3.79-3.75 (m, 3H). LCMS R_{T}=1.272 min, m/z=315.3 [M+H]⁺.

### Step 5: 2,2,2-trifluoro-1-(1-(4-methoxybenzyl)-6-oxo-1,6-dihydropyridazin-4-yl)ethyl methanesulfonate

To a solution of 2-(4-methoxybenzyl)-5-(2,2,2-trifluoro-1-hydroxyethyl)pyridazin-3(2H)-one (350 mg, 1.11 mmol) in methylene chloride (0.5 mL) was added triethylamine (338 mg, 3.34 mmol) and methane sulfonyl chloride (383 mg, 3.34 mmol) at 0° C. After stirred at 25° C. for 30 min under nitrogen atmosphere, the reaction mixture was quenched by addition of saturated sodium bicarbonate (3 mL) and was extracted with ethyl acetate (30 mL×3). The combined organic layers were washed with brine (30 mL), dried over anhydrous sodium sulfate and concentrated under reduced pressure to afford the title compound. It was used directly for next step without further purification. LCMS R_{T}=0.895 min, m/z=393.0 [M+H]⁺.

### Step 6: 2-(4-methoxybenzyl)-5-(2,2,2-trifluoroethyl)pyridazin-3(2H)-one

To a solution of 2,2,2-trifluoro-1-(1-(4-methoxybenzyl)-6-oxo-1,6-dihydropyridazin-4-yl)ethyl methanesulfonate (540 mg, 1.38 mmol) in methanol (25 mL) was added hydrochloric acid (2.0 mL, 12 M) and 10% palladium on carbon (0.2 g, 0.01 mmol). After stirred at 25° C. for 16 h under hydrogen atmosphere (15 psi), the reaction mixture was filtered and the filtrate was concentrated under reduced pressure. The residue was purified by column chromatography to afford the title compound. LCMS R_{T}=1.567 min, m/z=298.9 [M+H]⁺.

### Step 7: 5-(2,2,2-trifluoroethyl)pyridazin-3(2H)-one

To a mixture of 2-(4-methoxybenzyl)-5-(2,2,2-trifluoroethyl)pyridazin-3(2H)-one (92 mg, 0.3 mmol) in trifluoroacetic acid (1.5 mL) was added trifluoroacetic anhydride (0.75 mL). After stirred at 120° C. for 10 min, the reaction mixture was concentrated under reduced pressure. The residue was purified by preparative HPLC to afford the title compound. ¹H NMR (400 MHz, CDCl₃) δ=10.83-10.47 (m, 1H), 7.77-7.72 (m, 1H), 6.94-6.90 (m, 1H), 3.35-3.25 (m, 2H). LCMS R_{T}=0.308 min, m/z=178.9 [M+H]⁺.

### Step 8: (S)-tert-butyl 7-(6-chloro-8-(2-(((methylsulfonyl)oxy)methyl)thieno[3,2-b]pyridin-7-yl)-3,4-dihydroquinolin-1(2H)-yl)-5-azaspiro[3.4]octane-5-carboxylate

To a solution of (S)-tert-butyl 7-(6-chloro-8-(2-(hydroxymethyl)thieno[3,2-b]pyridin-7-yl)-3,4-dihydroquinolin-1(2H)-yl)-5-azaspiro[3.4]octane-5-carboxylate (50 mg, 0.09 mmol) in dichloromethane (2 mL) was added trimethylamine (23 mg, 0.23 mmol) and methanesulfonyl chloride (200 mg, 1.75 mmol). After stirred at 25° C. for 0.5 h, the mixture was quenched by addition of saturated sodium bicarbonate (3 mL) and extracted with dichloromethane (2 mL). The organic layer was washed with brine (3 mL), dried over anhydrous sodium sulfate and concentrated under reduced pressure to afford the title compound which was used in next step without further purification. LCMS R_{T}=3.163 min, m/z=618.2 [M+H]⁺.

### Step 9

To a solution of (R)-tert-butyl 7-(6-chloro-8-(2-(((methylsulfonyl)oxy)methyl)thieno[3,2-b]pyridin-7-yl)-3,4-dihydroquinolin-1(2H)-yl)-5-azaspiro[3.4]octane-5-carboxylate (50 mg, 0.08 mmol) in acetonitrile (4 mL) was added 5-(2,2,2-trifluoroethyl)pyridazin-3(2H)-one (25 mg, 0.14 mmol), potassium carbonate (27 mg, 0.2 mmol) and sodium iodide (6 mg, 0.04 mmol). After stirred at 80° C. for 2 h, the mixture was concentrated. The residue was diluted with water (15 mL) and extracted with ethyl acetate (15 mL×2). The combined organic layers were washed with brine (20 mL), dried over anhydrous sodium sulfate and concentrated under reduced pressure. The residue was added dioxane (2 mL) and hydrochloric acid (4 M in dioxane, 2.3 mL). After stirred at 25° C. for 0.5 h, the mixture was concentrated under reduced pressure. The residue was purified by preparative HPLC to afford Example 154. ¹H NMR (400 MHz, CD₃OD) δ=8.69 (d, J=4.8 Hz, 1H), 8.54 (s, 1H), 7.98 (d, J=1.6 Hz, 1H), 7.59 (s, 1H), 7.42 (d, J=4.8 Hz, 1H), 7.16-7.11 (m, 2H), 7.04 (s, 1H), 5.69-5.58 (m, 2H), 3.66-3.39 (m, 3H), 3.22-3.03 (m, 2H), 2.94-2.74 (m, 3H), 2.11-1.81 (m, 5H), 1.72-1.22 (m, 6H). LCMS R_{T}=1.567 min, m/z=600.4 [M+H]⁺.

### Example 155: (3aR,4S,7R,7aS)-2-((7-(6-chloro-1-((S)-5-azaspiro[3.4]octan-7-yl)-1,2,3,4-tetrahydroquinolin-8-yl)thieno[3,2-b]pyridin-2-yl)methyl)hexahydro-1H-4,7-methanoisoindole-1,3(2H)-dione, hydrochloride salt

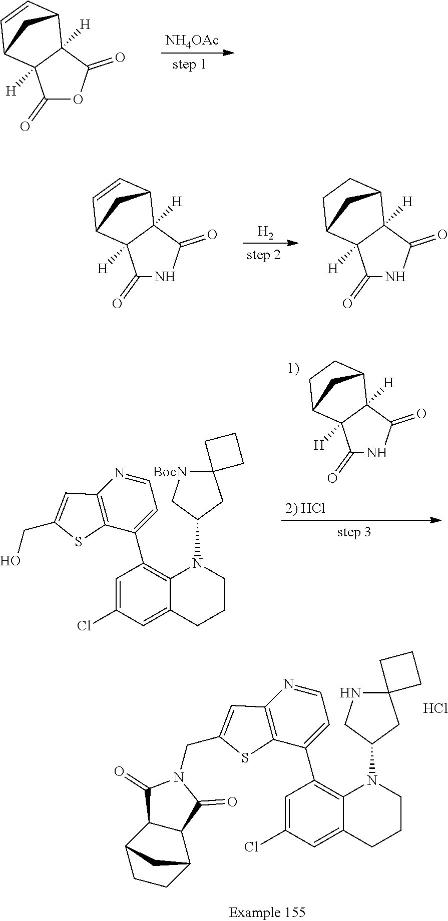

### Step 1: (3aR,4R,7S,7aS)-3a,4,7,7a-tetrahydro-1H-4,7-methanoisoindole-1,3(2H)-dione

To a solution of (3aR,4R,7S,7aS)-3a,4,7,7a-tetrahydro-4,7-methanoisobenzofuran-1,3-dione (1.0 g, 6.09 mmol) was added ammonium acetate (2.4 g, 30.46 mmol). After stirred at 135° C. for 16 h under nitrogen atmosphere, the reaction mixture was quenched by addition of saturated sodium bicarbonate, and extracted with ethyl acetate (30 mL×3). The combined organic layers were washed with brine (30 mL), dried over anhydrous sodium sulfate and concentrated under reduced pressure to afford the title compound. ¹H NMR (400 MHz, CD₃OD) δ=6.37-6.23 (m, 2H), 3.38-3.23 (m, 2H), 2.81-2.68 (m, 2H), 1.61-1.55 (m, 1H), 1.49-1.44 (m, 1H).

### Step 2: (3aR,4S,7R,7aS)-hexahydro-1H-4,7-methanoisoindole-1,3(2H)-dione

To the solution of (3aR,4R,7S,7aS)-3a,4,7,7a-tetrahydro-1H-4,7-methanoisoindole-1,3(2H)-dione (1.1 g, 6.93 mmol) in methanol (20 ml) was added 10% palladium on carbon (700 mg, 0.07 mmol). After stirred at 25° C. for 4 h under hydrogen atmosphere, the reaction mixture was concentrated under reduced pressure. The residue was purified by preparative HPLC to afford the title compound. ¹H NMR (400 MHz, CDCl₃) δ=8.60-7.96 (m, 1H), 6.43-6.27 (m, 2H), 3.42-3.26 (m, 2H), 2.83-2.69 (m, 2H), 1.61-1.53 (m, 1H), 1.49-1.42 (m, 1H).

### Step 3

Example 155 was prepared from (S)-tert-butyl 7-(6-chloro-8-(2-(hydroxymethyl)thieno[3,2-b]pyridin-7-yl)-3,4-dihydroquinolin-1(2H)-yl)-5-azaspiro[3.4]octane-5-carboxylate and (3aR,4S,7R,7aS)-hexahydro-1H-4,7-methanoisoindole-1,3(2H)-dione, following the procedure described in the synthesis of Example 108. The product was purified by preparative HPLC. ¹H NMR (400 MHz, CD₃OD) δ=9.10-8.89 (m, 1H), 8.15-7.96 (m, 1H), 7.88-7.69 (m, 1H), 7.39-7.25 (m, 2H), 5.13-5.02 (m, 2H), 3.67-3.49 (m, 1H), 3.31 (m, J=1.6, 3.2 Hz, 2H), 2.78 (s, 5H), 2.62 (m, 2H), 2.45-2.29 (m, 1H), 2.27-1.55 (m, 11H), 1.50-0.93 (m, 5H). LCMS R_{T}=1.617 min, m/z=587.4 [M+H]⁺.

### Example 156: (S)-5-((7-(6-chloro-1-(5-azaspiro[3.4]octan-7-yl)-1,2,3,4-tetrahydroquinolin-8-yl)thieno[3,2-b]pyridin-2-yl)methyl)-2,2-dimethyl-1,3-dioxane-4,6-dione

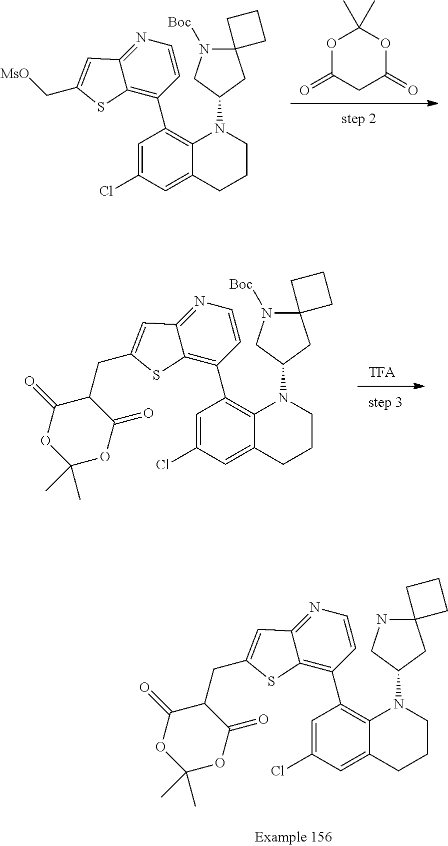

### Step 1: (S)-tert-butyl 7-(6-chloro-8-(2-((2,2-dimethyl-4,6-dioxo-1,3-dioxan-5-yl)methyl)thieno[3,2-b]pyridin-7-yl)-3,4-dihydroquinolin-1(2H)-yl)-5-azaspiro[3.4]octane-5-carboxylate

To a solution of (S)-tert-butyl 7-(6-chloro-8-(2-(((methylsulfonyl)oxy)methyl)thieno[3,2-b]pyridin-7-yl)-3,4-dihydroquinolin-1(2H)-yl)-5-azaspiro[3.4]octane-5-carboxylate (35 mg, 0.06 mmol) in DMF (1 mL) was added 2,2-dimethyl-1,3-dioxane-4,6-dione (12 mg, 0.08 mmol), potassium carbonate (23 mg, 0.17 mmol) and potassium iodide (2 mg, 0.01 mmol). After stirred at 60° C. for 16 h under nitrogen atmosphere, the reaction mixture was extracted with ethyl acetate (5 mL×3). The combined organic layers were washed with brine (5 mL), dried over anhydrous sodium sulfate and concentrated under reduced pressure. The residue was purified by preparative TLC to afford the title compound. LCMS R_{T}=1.035 min, m/z=666.0 [M+H]⁺.

### Step 2

To a solution of (S)-tert-butyl 7-(6-chloro-8-(2-((2,2-dimethyl-4,6-dioxo-1,3-dioxan-5-yl)methyl)thieno[3,2-b]pyridin-7-yl)-3,4-dihydroquinolin-1(2H)-yl)-5-azaspiro[3.4]octane-5-carboxylate (16 mg, 0.02 mmol) in dichloromethane (0.5 mL) was added trifluoroacetic acid (3 mg, 0.02 mmol) at 0° C. After stirred at 0° C. for 1 h, the reaction mixture was concentrated under reduced pressure. The residue was purified by preparative HPLC to afford Example 159. ¹H NMR (400 MHz, CD₃OD) δ=8.63-8.49 (m, 1H), 7.36-7.21 (m, 2H), 7.17-7.10 (m, 2H), 4.96-4.92 (m, 1H), 3.86-3.80 (m, 2H), 3.65-3.50 (m, 1H), 3.13 (d, J=1.6 Hz, 2H), 2.91-2.80 (m, 2H), 2.32-2.00 (m, 3H), 1.95-1.72 (m, 6H), 1.63 (s, 7H), 1.46-1.06 (m, 2H). LCMS R_{T}=1.224 min, m/z=566.4 [M+H]⁺.

### Example 157: (S)-3-((7-(6-chloro-1-((S)-5-azaspiro[3.4]octan-7-yl)-1,2,3,4-tetrahydroquinolin-8-yl)thieno[3,2-b]pyridin-2-yl)methyl)-4-(trifluoromethyl)oxazolidin-2-one, formic acid salt

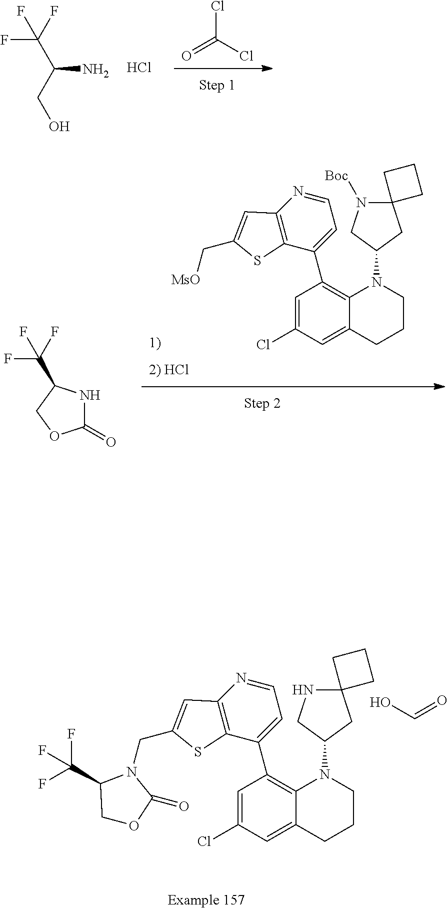

### Step 1: (S)-4-(trifluoromethyl)oxazolidin-2-one

To a solution of (S)-2-amino-3,3,3-trifluoropropan-1-ol (300 mg, 2.32 mmol) in toluene (5 mL) was added bis(trichloromethyl) carbonate (414 mg, 1.39 mmol) in toluene (5 mL) and potassium hydroxide (260 mg, 4.65 mmol) at 0° C. After stirred at 0° C. for 2 h, the mixture was added water (2 mL)/methanol (5 mL) and concentrated under reduced pressure. The residue was purified by preparative HPLC to afford the title compound. LCMS RT=0.240, m/z=156.1 [M+H]⁺.

### Step 2

To a mixture of (S)-4-(trifluoromethyl)oxazolidin-2-one (9 mg, 0.06 mmol), potassium iodide (16 mg, 0.10 mmol) and potassium carbonate (20 mg, 0.15 mmol) in DMF (3 mL) was added (S)-tert-butyl 7-(6-chloro-8-(2-(((methylsulfonyl)oxy)methyl)thieno[3,2-b]pyridin-7-yl)-3,4-dihydroquinolin-1(2H)-yl)-5-azaspiro[3.4]octane-5-carboxylate (30 mg, 0.05 mmol) at 25° C. After stirred at 25° C. for 1 h, the reaction mixture was concentrated under reduced pressure to remove the solvent. The residue was added hydrochloric acid (4 M in dioxane, 5 mL). After stirred at 25° C. for 30 min, the reaction mixture was concentrated under reduced pressure. The residue was purified by preparative HPLC to afford Example 160. ¹H NMR (400 MHz, CD₃OD) δ=8.74 (d, J=4.8 Hz, 1H), 8.53 (s, 1H), 7.57-7.46 (m, 2H), 7.20-7.15 (m, 2H), 4.88-4.86 (m, 2H), 4.70-4.62 (m, 1H), 4.58 (t, J=9.6 Hz, 1H), 4.52-4.45 (m, 1H), 3.61 (m, J=8.8 Hz, 1H), 3.29-3.12 (m, 2H), 2.98-2.79 (m, 2H), 2.27-2.14 (m, 1H), 2.03-0.73 (m, 11H). LCMS R_{T}=1.406 min, m/z=577.4 [M+H]⁺.

### Example 158: (R)-3-((7-(6-chloro-1-((S)-5-azaspiro[3.4]octan-7-yl)-1,2,3,4-tetrahydroquinolin-8-yl)thieno[3,2-b]pyridin-2-yl)methyl)-4-(trifluoromethyl)oxazolidin-2-one, formic acid salt

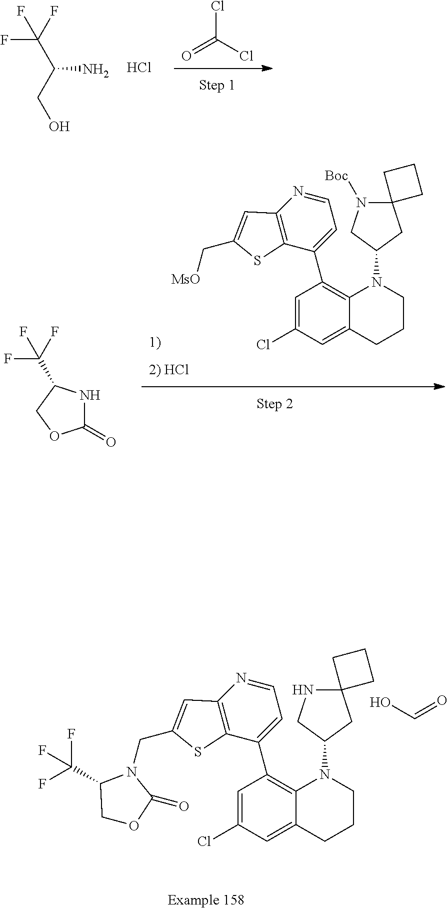

### Step 1: (R)-4-(trifluoromethyl)oxazolidin-2-one

To a solution of (R)-2-amino-3,3,3-trifluoropropan-1-ol (450 mg, 3.49 mmol) in toluene (5 mL) was added bis(trichloromethyl) carbonate (520 mg, 1.74 mmol) in toluene (5 mL) and potassium hydroxide (390 mg, 6.97 mmol) at 0° C. After stirred at 0° C. for 2 h, the mixture was added water (2 mL)/methanol (5 mL) and concentrated under reduced pressure. The residue was purified by preparative HPLC to afford the title compound. LCMS R_{T}=0.240, m/z=156.1 [M+H]⁺.

### Step 2

Example 158 was prepared from (R)-4-(trifluoromethyl)oxazolidin-2-one, following the procedure described in the synthesis of Example 157. The product was purified by preparative HPLC. ¹H NMR (400 MHz, CD₃OD) δ=8.74-8.70 (m, 1H), 8.54 (s, 1H), 7.57-7.51 (m, 1H), 7.49-7.44 (m, 1H), 7.17-7.12 (m, 2H), 4.84-4.83 (m, 2H), 4.54-4.45 (m, 3H), 3.58-3.52 (m, 1H), 3.21-3.13 (m, 2H), 2.89-2.83 (m, 2H), 2.10-1.83 (m, 5H), 1.91-1.83 (m, 1H), 1.74-1.46 (m, 6H). LCMS R_{T}=1.433 min, m/z=577.4 [M+H]⁺.

### Example 159: (S)-3-((7-(6-chloro-1-(5-azaspiro[3.4]octan-7-yl)-1,2,3,4-tetrahydroquinolin-8-yl)thieno[3,2-b]pyridin-2-yl)methyl)-1-(3,3-difluorocyclobutyl)imidazolidine-2,4-dione

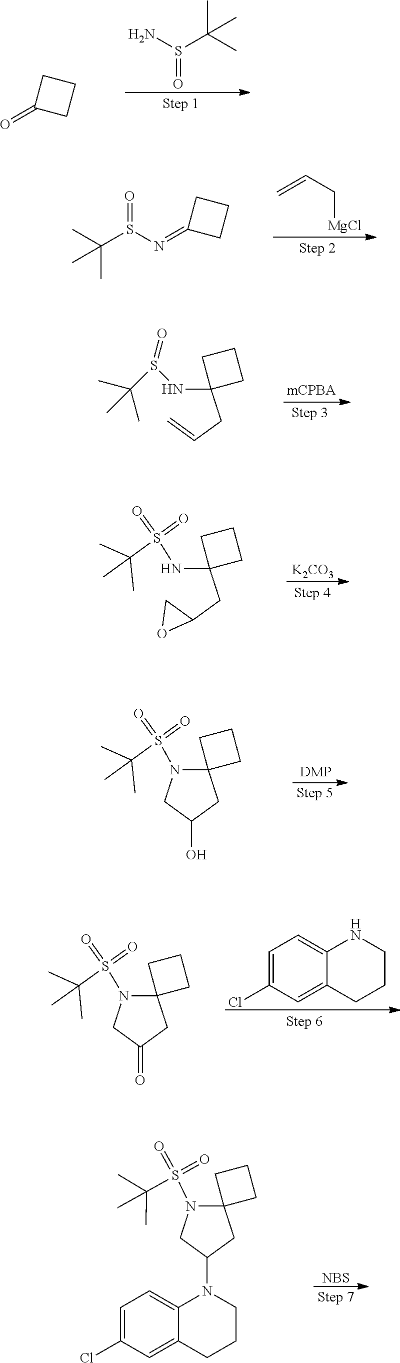

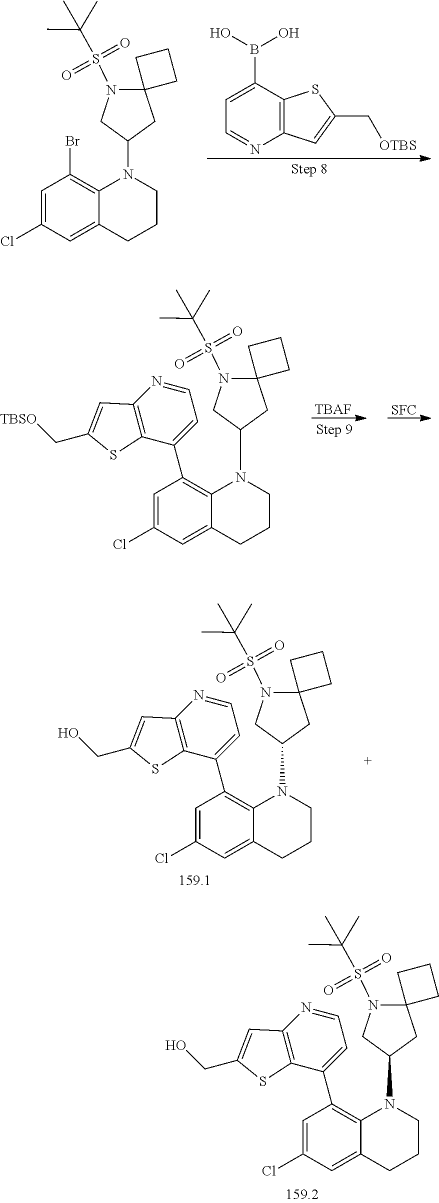

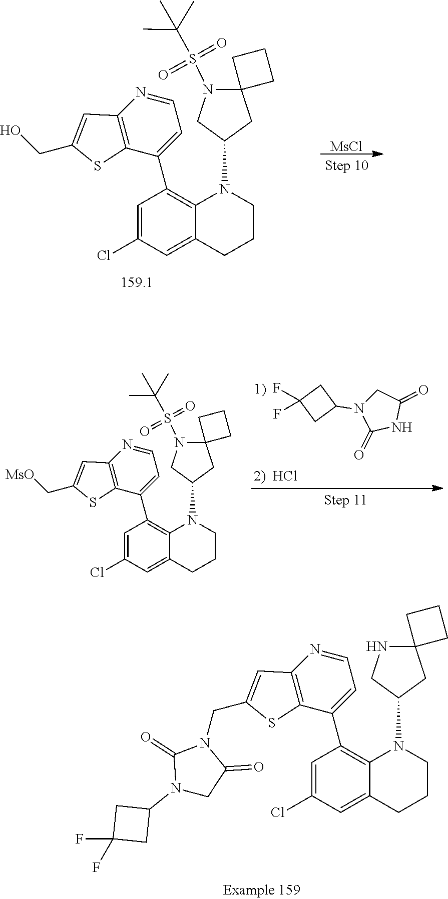 [SMILES: "MsCl" ; CC(C)(C)S(=O)(=O)N1C[C@@H](N2CCCc3cc(Cl)cc(-c4ccnc5cc(CO)sc45)c32)CC12CCC2 ; CC(C)(C)S(=O)(=O)N1C[C@@H](N2CCCc3cc(Cl)cc(-c4ccnc5cc(COS(C)(=O)=O)sc45)c32)CC12CCC2 ; O=C1CN(C2CC(F)(F)C2)C(=O)N1 ; O=C1CN(C2CC(F)(F)C2)C(=O)N1Cc1cc2nccc(-c3cc(Cl)cc4c3N([C@@H]3CNC5(CCC5)C3)CCC4)c2s1]

### Step 1: N-cyclobutylidene-2-methylpropane-2-sulfinamide

To a solution of cyclobutanone (60.0 g, 856.04 mmol) in THF (500 mL) was added 2-methylpropane-2-sulfinamide (114.0 g, 856.04 mmol) and titanium tetraisopropanolate (291.9 g, 1.03 mol). After stirred at 25° C. for 12 h, the reaction mixture was diluted with water (1000 mL) and filtered. The filtrate was concentrated under reduced pressure. The residue was purified by column chromatography to give the crude title compound. It was used in next step without further purification.

### Step 2: N-(1-allylcyclobutyl)-2-methylpropane-2-sulfinamide

To a solution of allylmagnesium bromide (99.6 mL, 2 M) in THF (100 mL) was added N-cyclobutylidene-2-methylpropane-2-sulfinamide (23.0 g, 132.74 mmol) in THF (100 mL). After stirred at −78° C. for 12 h, the residue was diluted with saturated ammonium chloride (150 mL), extracted with ethyl acetate (300 mL×3). The combined organic layers were washed with brine (300 mL), dried over anhydrous sodium sulfate and concentrated under reduced pressure. The residue was purified by column chromatography to give the title compound. LCMS R_{T}=0.678 min, m/z=216.0 [M+H]⁺

### Step 3: 2-methyl-N-(1-(oxiran-2-ylmethyl) cyclobutyl) propane-2-sulfonamide

To a solution of N-(1-allylcyclobutyl)-2-methylpropane-2-sulfinamide (21.0 g, 97.51 mmol) in dichloromethane (250 mL) was added 3-chloroperbenzoic acid (50.5 g, 292.54 mmol). After stirred at 20° C. for 16 h, the reaction mixture was diluted with saturated sodium bicarbonate (200 mL), the mixture was filtered and the filtrate was extracted with ethyl acetate (150 mL×3). The combined organic layers were washed with brine (250 mL), dried over anhydrous sodium sulfate and concentrated under reduced pressure. The concentrate was diluted with saturated sodium sulfite (200 mL), extracted with ethyl acetate (150 mL×3). The combined organic layers were washed with brine (250 mL), dried over anhydrous sodium sulfate and concentrated under reduced pressure. The residue was purified by column chromatography to give the title compound.

### Step 4: 5-(tert-butylsulfonyl)-5-azaspiro[3.4]octan-7-ol

To a solution of 2-methyl-N-(1-(oxiran-2-ylmethyl) cyclobutyl) propane-2-sulfonamide (23.5 g, 95.01 mmol) in N, N-dimethylformamide (230 mL) was added potassium carbonate (39.4 g, 285.02 mmol) and potassium iodide (15.8 g, 95.01 mmol). After stirred at 100° C. for 3 h, the reaction mixture was diluted with hydrochloric acid (1 M, 50 mL) and extracted with ethyl acetate (150 mL×3). The combined organic layers were washed with brine (250 mL), dried over anhydrous sodium sulfate and concentrated under reduced pressure. The residue was purified by column chromatography to give the title compound. ¹H NMR (400 MHz, CDCl₃) δ=4.45-4.33 (m, 1H), 3.77-3.62 (m, 1H), 3.46-3.30 (m, 1H), 3.07-2.83 (m, 2H), 2.43 (br, 2H), 2.20-2.07 (m, 2H), 1.95-1.84 (m, 1H), 1.79-1.66 (m, 1H), 1.64-1.51 (m, 1H), 1.48-1.36 (m, 9H).

### Step 5: 5-(tert-butylsulfonyl)-5-azaspiro[3.4]octan-7-one

To a solution of 5-(tert-butylsulfonyl)-5-azaspiro[3.4]octan-7-ol (16.9 g, 68.32 mmol) in dichloromethane (200 mL) was added Dess-Martin periodinane (37.7 g, 88.82 mmol). After stirred at 25° C. for 12 h, the reaction mixture was filtered. Then the filtrate was diluted with saturated aqueous sodium bicarbonate (500 mL), extracted with dichloromethane (400 mL×3). The combined organic layers were washed with brine (400 mL), dried over anhydrous sodium sulfate and concentrated under reduced pressure. The residue was purified by column chromatography to give the title compound. ¹H NMR (400 MHz, CDCl₃) δ=3.86-3.80 (m, 2H), 3.08-2.98 (m, 2H), 2.77 (s, 2H), 2.09-2.02 (m, 2H), 1.94-1.84 (m, 1H), 1.71-1.59 (m, 1H), 1.43-1.41 (m, 9H) Step 6: 1-(5-(tert-butylsulfonyl)-5-azaspiro[3.4]octan-7-yl)-6-chloro-1,2,3,4-tetrahydroquinoline

To a solution of 5-tert-butylsulfonyl-5-azaspiro[3.4]octan-7-one (10.0 g, 40.76 mmol) in methanol (100 mL) were added 6-chloro-1,2,3,4-tetrahydroquinoline (5.7 g, 33.97 mmol) and triethylsilane (15.6 g, 67.93 mmol) and indium (III) chloride (2.3 g, 10.19 mmol). After stirred at 20° C. for 16 h, the reaction mixture was concentrated under reduced pressure. The residue was purified by column chromatography to give the title compound. LCMS R_{T}=1.147 min, m/z=397.1 [M+H]⁺

### Step 7: 8-bromo-1-(5-(tert-butylsulfonyl)-5-azaspiro[3.4]octan-7-yl)-6-chloro-1,2,3,4-tetrahydroquinoline

To a solution of 1-(5-tert-butylsulfonyl-5-azaspiro[3.4]octan-7-yl)-6-chloro-3,4-dihydro-2H-quinoline (2.8 g, 7.05 mmol) in DMF (20 mL) was added 1-bromopyrrolidine-2,5-dione (1.5 g, 8.46 mmol) at 0° C. After stirred at 25° C. for 1 h, the reaction mixture was concentrated under reduced pressure. The residue was purified by column chromatography to afford the title compound. LCMS R_{T}=0.677 min, m/z=477.1 [M+H]⁺.

### Step 8: 2-(((tert-butyldimethylsilyl)oxy)methyl)-7-(1-(5-(tert-butylsulfonyl)-5-azaspiro[3.4]octan-7-yl)-6-chloro-1,2,3,4-tetrahydroquinolin-8-yl)thieno[3,2-b]pyridine

To a solution of 8-bromo-1-(5-tert-butylsulfonyl-5-azaspiro[3.4]octan-7-yl)-6-chloro-3,4-dihydro-2H-quinoline (0.7 g, 1.47 mmol) and [2-[[tert-butyl(dimethyl)silyl]oxymethyl]thieno[3,2-b]pyridin-7-yl]boronic acid (0.7 g, 2.21 mmol) in dioxane (10 mL)/water (1 mL) was added Cesium carbonate (1.44 g, 4.41 mmol) and Palladium dichloride[1,1′-bis (diphenylphosphine) ferrocene](0.1 g, 0.10 mmol) at 25° C. After stirred at 100° C. for 3 h, the reaction mixture was concentrated under reduced pressure. The residue was purified by column chromatography to afford the title compound. LCMS R_{T}=0.675 min, m/z=674.3 [M+H]⁺.

### Step 9: (R)-(7-(1-(5-(tert-butylsulfonyl)-5-azaspiro[3.4]octan-7-yl)-6-chloro-1,2,3,4-tetrahydroquinolin-8-yl)thieno[3,2-b]pyridin-2-yl)methanol and (S)-(7-(1-(5-(tert-butylsulfonyl)-5-azaspiro[3.4]octan-7-yl)-6-chloro-1,2,3,4-tetrahydroquinolin-8-yl)thieno[3,2-b]pyridin-2-yl)methanol

To a solution of tert-butyl-[[7-[1-(5-tert-butylsulfonyl-5-azaspiro[3.4]octan-7-yl)-6-chloro-3,4-dihydro-2H-quinolin-8-yl]thieno[3,2-b]pyridin-2-yl]methoxy]-dimethyl-silane (100 mg, 0.14 mmol) in THF (1 mL) was added tetrabutylammonium fluoride (0.2 mL, 1 M in THF) at 25° C. After stirred at 25° C. for 0.5 h, the reaction mixture was concentrated under reduced pressure. The residue was purified by preparative TLC. The racemate was separated by SFC to give first eluting fraction (162.1, Rₜ=2.874 min; LCMS R_{T}=0.950 min, m/z=560.0 [M+H]*) and second eluting fraction (162.2, Rt=3.557 min; LCMS R_{T}=0.947 min, m/z=560.0 [M+H]+).

### Step 10: (S)-(7-(1-(5-(tert-butylsulfonyl)-5-azaspiro[3.4]octan-7-yl)-6-chloro-1,2,3,4-tetrahydroquinolin-8-yl)thieno[3,2-b]pyridin-2-yl)methyl methanesulfonate

To a solution of 159.1 (40 mg, 0.07 mmol) in methylene chloride (4 mL) was added methanesulfonyl chloride (24 mg, 0.21 mmol) and triethylamine (21 mg, 0.21 mmol) at 0° C. After stirred at 25° C. for 1 h, the reaction mixture was quenched by addition of sodium bicarbonate (5 mL), extracted by methylene chloride (10 mL×4). The organic layers were dried over anhydrous sodium sulfate, filtered and the filtrate was concentrated under reduced pressure to afford the title compound, which was used in next step without further purification. LCMS R_{T}=0.612 min, m/z=638.1 [M+H]⁺

### Step 11

To a solution of (S)-(7-(1-(5-(tert-butylsulfonyl)-5-azaspiro[3.4]octan-7-yl)-6-chloro-1,2,3,4-tetrahydroquinolin-8-yl)thieno[3,2-b]pyridin-2-yl)methyl methanesulfonate (40 mg, 0.06 mmol) in DMF (2 mL) was added 1-(3,3-difluorocyclobutyl)imidazolidine-2,4-dione (23 mg, 0.12 mmol), potassium iodide (20 mg, 0.12 mmol) and potassium hydroxide (10 mg, 0.18 mmol) at 25° C. After stirred at 60° C. for 1 h, the reaction mixture was concentrated under reduced pressure. The residue was dissolved in hydrochloric acid (1 mL, 4 M in dioxane) was added. After stirred at 25° C. for 0.5 h, the reaction mixture was concentrated under reduced pressure. The residue was purified by preparative HPLC to afford Example 159. ¹H NMR (400 MHz, CD₃OD) δ=8.69 (d, J=4.8 Hz, 1H), 7.51 (s, 1H), 7.46-7.39 (m, 1H), 7.18-7.09 (m, 2H), 4.96 (s, 2H), 4.59 (s, 4H), 4.44 (d, J=4.8 Hz, 1H), 4.13 (s, 2H), 3.52 (s, 1H), 3.24-3.10 (m, 2H), 2.94-2.80 (m, 5H), 2.11 (d, J=9.6 Hz, 1H), 1.91 (s, 3H), 1.69 (s, 4H), 0.85 (t, J=7.2 Hz, 1H). LCMS R_{T}=0.512 min, m/z=612.4 [M+H]⁺.

### Example 160: 3-((7-(6-chloro-1-(5-azaspiro[3.4]octan-7-yl)-1,2,3,4-tetrahydroquinolin-8-yl)thieno[3,2-b]pyridin-2-yl)methyl)-1-methylpyrazin-2(1H)-one, formic acid salt

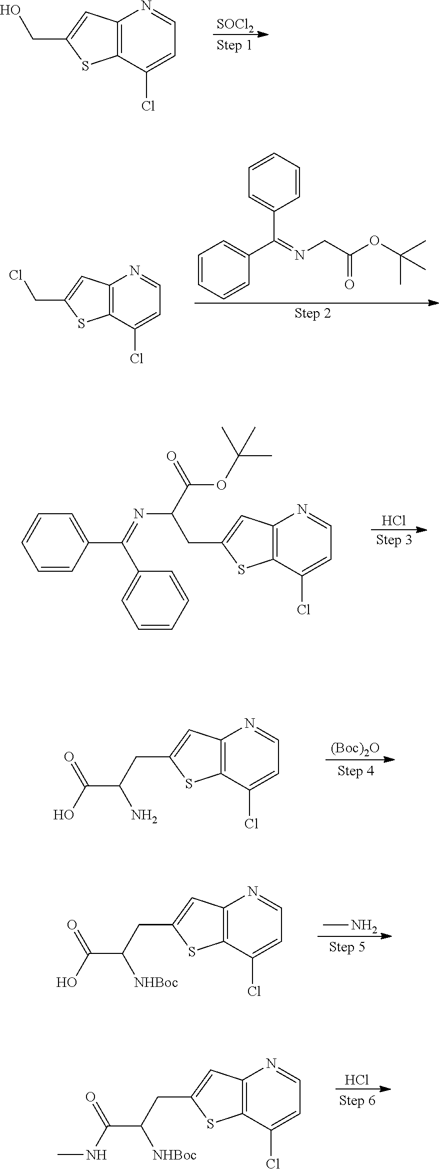

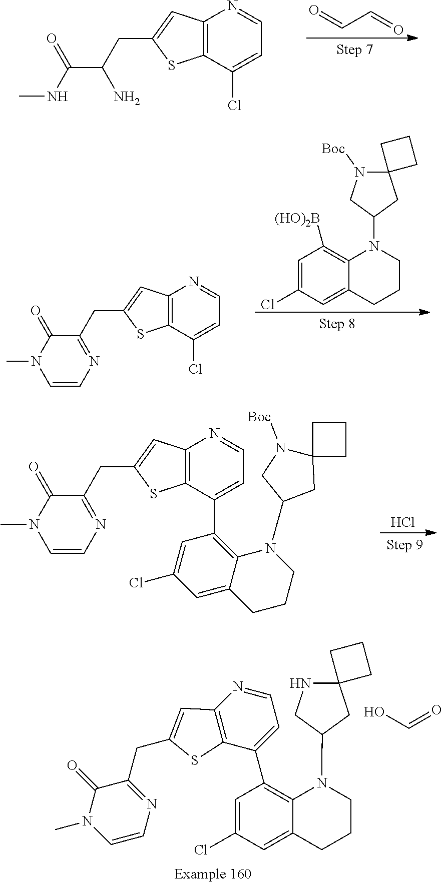

### Step 1: 7-chloro-2-(chloromethyl)thieno[3,2-b]pyridine

To a solution of (7-chlorothieno[3,2-b]pyridin-2-yl)methanol (14.0 g, 70.12 mmol) in dichloromethane (200 mL) was added sulfurous dichloride (25.0 g, 210.36 mmol). After stirred at 20° C. for 3 h, the mixture was quenched by addition of saturated aqueous sodium carbonate (150 mL) and filtered. The filtrate was extracted with dichloromethane (100 mL). The organic layer was washed with brine (50 mL), dried over anhydrous sodium sulfate and concentrated under reduced pressure. The residue was purified by column chromatography to afford the title compound. LCMS R_{T}=1.175 min, m/z=218.1 [M+H]⁺.

### Step 2: tert-butyl 3-(7-chlorothieno[3,2-b]pyridin-2-yl)-2-((diphenylmethylene)amino)propanoate

To a solution of 7-chloro-2-(chloromethyl)thieno[3,2-b]pyridine (4.9 g, 22.47 mmol) and tert-butyl 2-((diphenylmethylene)amino)acetate (6.9 g, 23.59 mmol) in toluene (120 mL) and dichloromethane (50 mL) was added tetrabutylammonium bromide (724 mg, 2.25 mmol) and potassium hydroxide (25.2 g, 449.33 mmol) in water (25 mL). After stirred at 20° C. for 8 h, the mixture was concentrated to remove dichloromethane and extracted with ethyl acetate (50 mL). The organic layer was washed with brine (50 mL), dried over anhydrous sodium sulfate and concentrated under reduced pressure. The residue was purified by column chromatography to afford the title compound. LCMS R_{T}=2.590 min, m/z=477.2 [M+H]⁺.

### Step 3: 2-amino-3-(7-chlorothieno[3,2-b]pyridin-2-yl)propanoic acid

A solution of tert-butyl 3-(7-chlorothieno[3,2-b]pyridin-2-yl)-2-((diphenylmethylene)amino)propanoate (8.7 g, 18.24 mmol) in hydrochloric acid (76.0 mL, 12 M) was stirred at 20° C. for 2 h. The mixture was concentrated under reduced pressure to afford the title compound which was used in next step without further purification. LCMS R_{T}=0.490 min, m/z=257.0 [M+H]⁺.

### Step 4: 2-((tert-butoxycarbonyl)amino)-3-(7-chlorothieno[3,2-b]pyridin-2-yl)propanoic acid

To a solution of 2-amino-3-(7-chlorothieno[3,2-b]pyridin-2-yl)propanoic acid (9.0 g, 21.04 mmol) in THF (50 mL) and water (50 mL) was added triethylamine (10.6 g, 105.18 mmol) and di-tert-butyl dicarbonate (9.2 g, 42.07 mmol). After stirred at 20° C. for 4 h, the mixture was concentrated to remove organic solvent and washed with ethyl acetate (20 mL). The aqueous layer was concentrated under reduced pressure to afford the title compound which was used in next step without further purification. LCMS R_{T}=1.112 min, m/z=357.2 [M+H]⁺.

### Step 5: tert-butyl (3-(7-chlorothieno[3,2-b]pyridin-2-yl)-1-(methylamino)-1-oxopropan-2-yl)carbamate

To a solution of 2-((tert-butoxycarbonyl)amino)-3-(7-chlorothieno[3,2-b]pyridin-2-yl)propanoic acid (0.6 g, 1.68 mmol) in DMF (15 mL) was added N-ethyl-N-isopropylpropan-2-amine (543 mg, 4.20 mmol), 2-(3H-[1,2,3]triazolo[4,5-b]pyridin-3-yl)-1,1,3,3-tetramethylisouronium hexafluorophosphate(V) (831 mg, 2.19 mmol) and methanamine hydrochloride (204 mg, 3.03 mmol). After stirred at 20° C. for 2 h, the mixture was diluted with ethyl acetate (100 mL) and washed with brine (100 mL×2). The organic layer was dried over anhydrous sodium sulfate and concentrated under reduced pressure. The residue was purified by column chromatography to afford the title compound. LCMS R_{T}=0.827 min, m/z=370.1 [M+H]⁺.

### Step 6: 2-amino-3-(7-chlorothieno[3,2-b]pyridin-2-yl)-N-methylpropanamide

A solution of tert-butyl (3-(7-chlorothieno[3,2-b]pyridin-2-yl)-1-(methylamino)-1-oxopropan-2-yl)carbamate (250 mg, 0.68 mmol) in hydrochloric acid (10 mL, 4 M in methanol) was stirred at 20° C. for 1 h. The mixture was concentrated under reduced pressure to afford the title compound which was used in next step without further purification. LCMS R_{T}=0.527 min, m/z=270.3 [M+H]⁺.

### Step 7: 3-((7-chlorothieno[3,2-b]pyridin-2-yl)methyl)-1-methylpyrazin-2(1H)-one

To a solution of 2-amino-3-(7-chlorothieno[3,2-b]pyridin-2-yl)-N-methylpropanamide (200 mg, 0.65 mmol) and oxalaldehyde (94 mg, 1.63 mmol) in methanol (10 mL) was added sodium hydroxide (52 mg, 1.31 mmol) at 0° C. After stirred at 0° C. for 1.5 h, the mixture was purified by column chromatography to afford the title compound. LCMS R_{T}=1.300 min, m/z=292.1 [M+H]⁺.

### Step 8: tert-butyl 7-(6-chloro-8-(2-((4-methyl-3-oxo-3,4-dihydropyrazin-2-yl)methyl)thieno[3,2-b]pyridin-7-yl)-3,4-dihydroquinolin-1(2H)-yl)-5-azaspiro[3.4]octane-5-carboxylate

To a solution of 3-((7-chlorothieno[3,2-b]pyridin-2-yl)methyl)-1-methylpyrazin-2(1H)-one (40 mg, 0.14 mmol) and (1-(5-(tert-butoxycarbonyl)-5-azaspiro[3.4]octan-7-yl)-6-chloro-1,2,3,4-tetrahydroquinolin-8-yl)boronic acid (86 mg, 0.21 mmol) in water (0.1 mL) and dioxane (1.5 mL) was added dichloro[1,1′-bis(diphenylphosphino)ferrocene]palladium(II) (20 mg, 0.027 mmol) and cesium carbonate (89 mg, 0.027 mmol). After stirred at 130° C. for 2 h in microwave under nitrogen atmosphere, the mixture was concentrated under reduced pressure. The residue was purified by preparative TLC to afford the title compound. LCMS R_{T}=0.993 min, m/z=632.1 [M+H]⁺.

### Step 9

A solution of tert-butyl 7-(6-chloro-8-(2-((4-methyl-3-oxo-3,4-dihydropyrazin-2-yl)methyl)thieno[3,2-b]pyridin-7-yl)-3,4-dihydroquinolin-1(2H)-yl)-5-azaspiro[3.4]octane-5-carboxylate (30 mg, 0.047 mmol) in hydrochloric acid (5 mL, 4 M in dioxane) was stirred at 20° C. for 1 h. The mixture was concentrated under reduced pressure. The residue was purified by preparative HPLC to afford Example 163. ¹H NMR (400 MHz, DMSO-d₆) δ=8.65 (d, J=4.8 Hz, 1H), 8.24 (s, 1H), 7.61 (d, J=4.4 Hz, 1H), 7.43-7.26 (m, 2H), 7.24-7.02 (m, 3H), 4.38-4.29 (m, 2H), 3.39-2.94 (m, 9H), 2.85-2.64 (m, 2H), 1.87-1.19 (m, 10H). LCMS R_{T}=1.210 min, m/z=532.4 [M+H]⁺.

### Example 161a and 161b: (S)-1-((4-(6-chloro-1-(5-azaspiro[3.4]octan-7-yl)-1,2,3,4-tetrahydroquinolin-8-yl)thieno[3,2-d]pyrimidin-6-yl)methyl)pyrrolidine-2,5-dione, formic acid salt and (R)-1-((4-(6-chloro-1-(5-azaspiro[3.4]octan-7-yl)-1,2,3,4-tetrahydroquinolin-8-yl)thieno[3,2-d]pyrimidin-6-yl)methyl)pyrrolidine-2,5-dione, formic acid salt

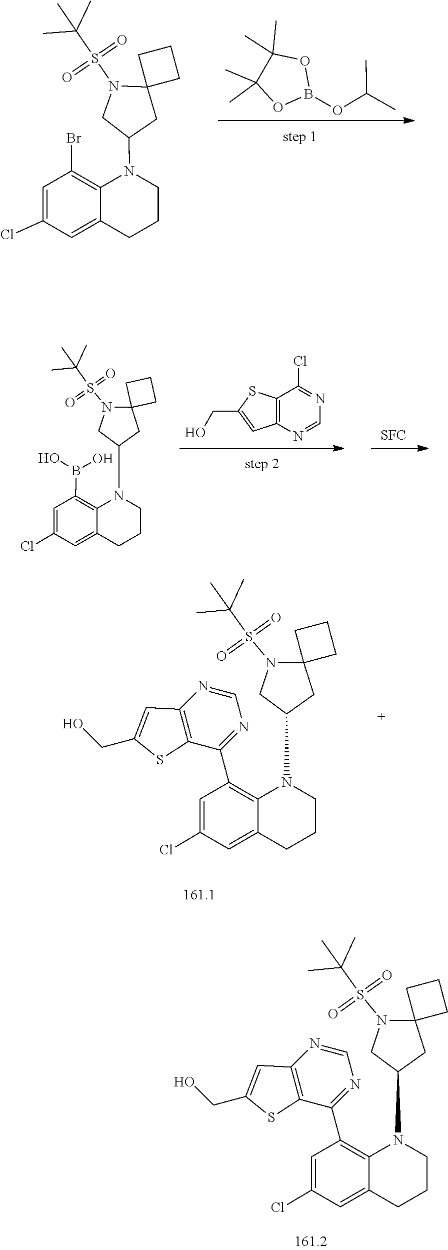

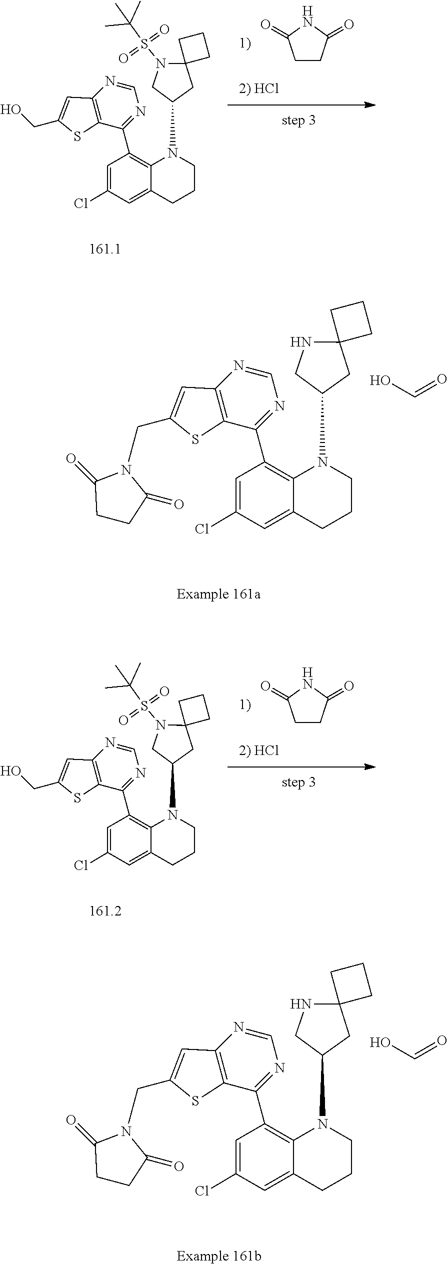

### Step 1: (1-(5-(tert-butylsulfonyl)-5-azaspiro[3.4]octan-7-yl)-6-chloro-1,2,3,4-tetrahydroquinolin-8-yl)boronic acid

To a solution of 8-bromo-1-(5-(tert-butylsulfonyl)-5-azaspiro[3.4]octan-7-yl)-6-chloro-1,2,3,4-tetrahydroquinoline (830 mg, 1.74 mmol) in THF (2 mL) was added lithium chloro(isopropyl)magnesium chloride (1.3 M in THF, 6.7 mL, 8.7 mmol) at 0° C. After stirred at 0° C. for 0.5 h, the mixture was added 2-isopropoxy-4,4,5,5-tetramethyl-1,3,2-dioxaborolane (973 mg, 5.23 mmol). After stirred at 20° C. for 0.5 h, the mixture was quenched by addition of water (10 mL), then extracted with ethyl acetate (5 mL×4). The combined organic layers were washed with brine (20 mL), dried over anhydrous sodium sulfate and concentrated under reduced pressure to afford the title compound which was used in next step without further purification. LCMS R_{T}=0.549 m, m/z=441.2 [M+H]⁺

### Step 2: (S)-(4-(1-(5-(tert-butylsulfonyl)-5-azaspiro[3.4]octan-7-yl)-6-chloro-1,2,3,4-tetrahydroquinolin-8-yl)thieno[3,2-d]pyrimidin-6-yl)methanol and (R)-(4-(1-(5-(tert-butylsulfonyl)-5-azaspiro[3.4]octan-7-yl)-6-chloro-1,2,3,4-tetrahydroquinolin-8-yl)thieno[3,2-d]pyrimidin-6-yl)methanol

To a solution of (1-(5-(tert-butylsulfonyl)-5-azaspiro[3.4]octan-7-yl)-6-chloro-1,2,3,4-tetrahydroquinolin-8-yl)boronic acid (1.1 g, 2.12 mmol) in THF (5 mL) was added (4-chlorothieno[3,2-d]pyrimidin-6-yl)methanol (638 mg, 3.18 mmol), [1,1-bis(diphenylphosphino)ferrocene]dichloropalladium(II) (388 mg, 0.53 mmol) and potassium trimethylsilanolate (408 mg, 3.18 mmol) at 20° C. After stirred at 60° C. for 2 h, the reaction mixture was filtered and the filtrate was concentrated under reduced pressure to remove solvent. The residue was purified by column chromatography. The racemate was separated by SFC to give first eluting fraction (164.1, Rt=3.977 min, LCMS R_{T}=0.576 min, m/z=561.3 [M+H]+) and second eluting fraction (164.2, Rt=4.265 min, LCMS R_{T}=0.576 min, m/z=561.3 [M+H]+).

### Step 3

Example 161a was prepared from 161.1 and succinimide, following the procedure described in the synthesis of Example 108. The product was purified by preparative HPLC. ¹H NMR (400 MHz, CD₃OD) δ=9.26-9.20 (m, 1H), 8.50 (s, 1H), 7.54 (s, 1H), 7.32 (d, J=2.8 Hz, 1H), 7.24 (d, J=2.4 Hz, 1H), 5.02 (d, J=0.8 Hz, 2H), 3.57-3.46 (m, 1H), 3.29-3.16 (m, 2H), 2.95-2.74 (m, 8H), 2.30-1.63 (m, 10H). LCMS R_{T}=1.316 m, m/z=522.3 [M+H]+

Example 161b was prepared from 161.2 and succinimide, following the procedure described in the synthesis of Example 108. The product was purified by preparative HPLC. ¹H NMR (400 MHz, CD₃OD) δ=9.28-9.15 (m, 1H), 8.52 (s, 1H), 7.53 (s, 1H), 7.31 (d, J=2.8 Hz, 1H), 7.22 (d, J=2.4 Hz, 1H), 5.01 (s, 2H), 3.49 (q, J=8.8 Hz, 1H), 3.28-3.15 (m, 2H), 2.99-2.65 (m, 8H), 2.25-1.52 (m, 10H). LCMS R_{T}=1.312 m, m/z=522.3 [M+H]+

The absolute configuration was tentatively assigned based on the relative potency of the pair diastereomers in biology assays.

### Example 162a and 162b: (S)-3-((7-(1-(5-azaspiro[3.4]octan-7-yl)-6-(trifluoromethyl)-1,2,3,4-tetrahydroquinolin-8-yl)thieno[3,2-b]pyridin-2-yl)methyl)-1-cyclopropylpyrimidine-2,4(1H,3H)-dione, formic acid salt and (R)-3-((7-(1-(5-azaspiro[3.4]octan-7-yl)-6-(trifluoromethyl)-1,2,3,4-tetrahydroquinolin-8-yl)thieno[3,2-b]pyridin-2-yl)methyl)-1-cyclopropylpyrimidine-2,4(1H,3H)-dione

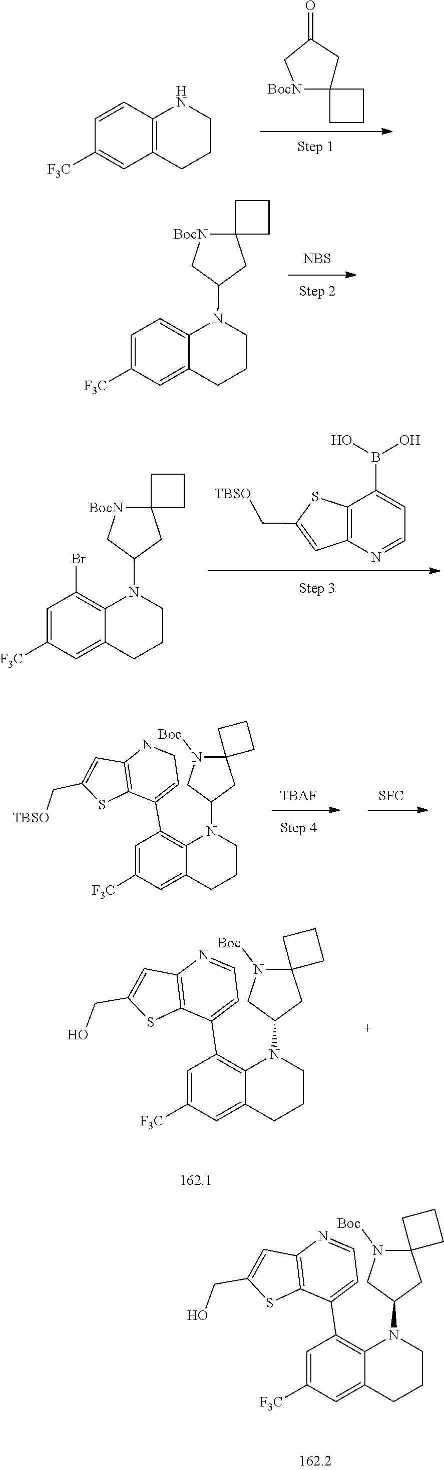

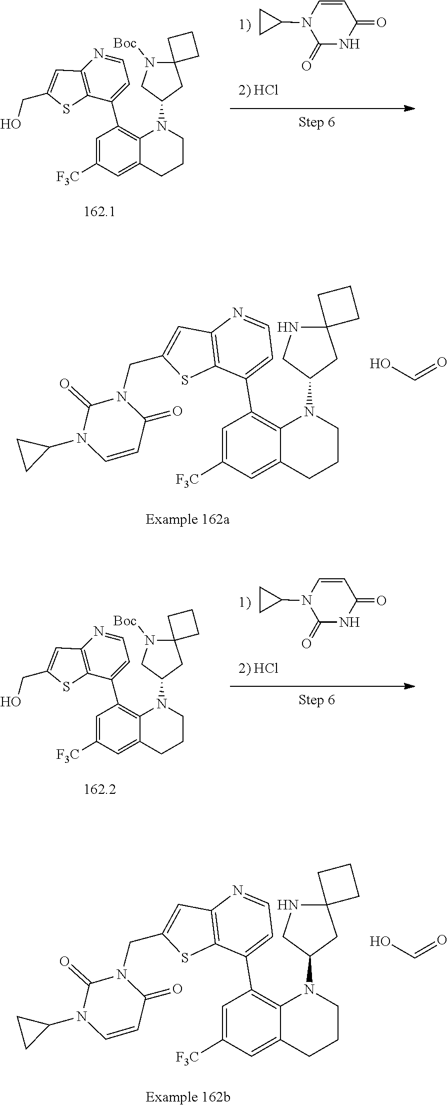

### Step 1: tert-butyl 7-(6-(trifluoromethyl)-3,4-dihydroquinolin-1(2H)-yl)-5-azaspiro[3.4]octane-5-carboxylate

To a solution of 6-(trifluoromethyl)-1,2,3,4-tetrahydroquinoline (400 mg, 1.99 mmol) and in acetic acid (5 mL) was added sodium borohydride acetate (1.1 g, 4.97 mmol) and tert-butyl 7-oxo-5-azaspiro[3.4]octane-5-carboxylate (373 mg, 1.66 mmol) at 25° C. After stirred at 25° C. for 12 h., the reaction mixture was extracted with ethyl acetate (20 mL×3). The combined organic layers were washed with brine (30 mL), dried over anhydrous sodium sulfate and concentrated under reduced pressure. The residue was purified by pre-TLC to afford the title compound. LCMS R_{T}=1.132 min, m/z=411.1 [M+H]⁺.

### Step 2: tert-butyl 7-(8-bromo-6-(trifluoromethyl)-3,4-dihydroquinolin-1(2H)-yl)-5-azaspiro[3.4]octane-5-carboxylate

To a solution of tert-butyl 7-(6-(trifluoromethyl)-3,4-dihydroquinolin-1(2H)-yl)-5-azaspiro[3.4]octane-5-carboxylate (300 mg, 0.73 mmol) in dichloromethane (5 mL) was added N-bromosuccinimide (117 mg, 0.66 mmol). After stirred at 25° C. for 4 h, the reaction mixture was extracted with ethyl acetate (10 mL×3). The combined organic layers were washed with brine (20 mL), dried over anhydrous sodium sulfate and concentrated under reduced pressure. The residue was purified by preparative TLC to afford the title compound. LCMS R_{T}=1.291 min, m/z=489.1 [M+H]⁺.

### Step 3: tert-butyl 7-(8-(2-(((tert-butyldimethylsilyl)oxy)methyl)thieno[3,2-b]pyridin-7-yl)-6-(trifluoromethyl)-3,4-dihydroquinolin-1(2H)-yl)-5-azaspiro[3.4]octane-5-carboxylate

To a solution of tert-butyl 7-(8-bromo-6-(trifluoromethyl)-3,4-dihydroquinolin-1(2H)-yl)-5-azaspiro[3.4]octane-5-carboxylate (120 mg, 0.25 mmol) in dioxane (5 mL) was added (2-(((tert-butyldimethylsilyl)oxy)methyl)thieno[3,2-b]pyridin-7-yl)boronic acid (95 mg, 0.29 mmol) and cesium carbonate (240 mg, 0.74 mmol) and water (1 mL) and dichloro[1,1′-bis(diphenylphosphino)ferrocene]palladium(II) (36 mg, 0.05 mol). After stirred at 100° C. for 1 h under nitrogen atmosphere, the reaction mixture was diluted with water (30 mL). The mixture was extracted with ethyl acetate (30 mL×2). The combined organic layers were washed with brine (30 mL), dried over anhydrous sodium sulfate and concentrated under reduced pressure. The residue was purified by column chromatography to afford the title compound. LCMS R_{T}=3.203 min, m/z=688.4 [M+H]⁺

### Step 4: (S)-tert-butyl 7-(8-(2-(hydroxymethyl)thieno[3,2-b]pyridin-7-yl)-6-(trifluoromethyl)-3,4-dihydroquinolin-1(2H)-yl)-5-azaspiro[3.4]octane-5-carboxylate and (R)-tert-butyl 7-(8-(2-(hydroxymethyl)thieno[3,2-b]pyridin-7-yl)-6-(trifluoromethyl)-3,4-dihydroquinolin-1(2H)-yl)-5-azaspiro[3.4]octane-5-carboxylate

To a solution of tert-butyl 7-(8-(2-(((tert-butyldimethylsilyl)oxy)methyl)thieno[3,2-b]pyridin-7-yl)-6-(trifluoromethyl)-3,4-dihydroquinolin-1(2H)-yl)-5-azaspiro[3.4]octane-5-carboxylate (110 mg, 0.16 mmol) in THF (5 mL) was added tetrabutylammonium fluoride (1.5 mL, 1.0 M in THF). After stirred for 30 min at 25° C., the reaction mixture was concentrated under reduced pressure. The residue was purified by column chromatography. The racemate was separated by SFC to give first eluting fraction (162.1, Rt=1.806 min, LCMS R_{T}=2.102 min, m/z=574.4 [M+H]+), second eluting fraction (162.2, Rt=2.031 min, LCMS R_{T}=2.102 min, m/z=574.4 [M+H]+).

### Step 6

Example 162a was prepared from 162.1 and 1-cyclopropylpyrimidine-2,4(1H,3H)-dione, following the procedure described in the synthesis of Example 78. The product was purified by preparative HPLC. ¹H NMR (400 MHz, CD₃OD) δ=8.72 (d, J=4.8 Hz, 1H), 8.54 (s, 1H), 7.60 (d, J=8.0 Hz, 2H), 7.51-7.33 (m, 3H), 5.73 (d, J=8.0 Hz, 1H), 5.46-5.32 (m, 2H), 3.78-3.52 (m, 1H), 3.12 (tt, J=3.6, 7.2 Hz, 5H), 2.62-2.29 (m, 5H), 2.02-1.91 (m, 1H), 1.80-1.20 (m, 6H), 1.04 (q, J=6.8 Hz, 2H), 0.89-0.83 (m, 2H). LCMS R_{T}=1.817 min, m/z=608.3 [M+H]+

Example 162b was prepared from 162.2 and 1-cyclopropylpyrimidine-2,4-dione (12 mg, 0.08 mmol), following the procedure described in the synthesis of Example 78. The product was purified by preparative HPLC. ¹H NMR (400 MHz, CD₃OD) δ=8.71 (d, J=4.8 Hz, 1H), 8.54 (s, 1H), 7.60 (d, J=8.0 Hz, 2H), 7.48-7.32 (m, 3H), 5.73 (d, J=8.0 Hz, 1H), 5.47-5.31 (m, 2H), 3.62 (s, 1H), 3.25-2.67 (m, 5H), 2.54-2.24 (m, 1H), 2.23-1.80 (m, 5H), 1.80-1.20 (m, 6H), 1.12-0.93 (m, 2H), 0.89-0.81 (m, 2H). LCMS R_{T}=1.512 min, m/z=608.4 [M+H]+

The absolute configuration was tentatively assigned based on the relative potency of the pair diastereomers in biology assays.

### Example 163: (S)-1-((7-(1-(5-azaspiro[3.4]octan-7-yl)-6-(trifluoromethyl)-1,2,3,4-tetrahydroquinolin-8-yl)thieno[3,2-b]pyridin-2-yl)methyl)pyrrolidine-2,5-dione, formic acid salt

Prepared from (S)-tert-butyl 7-(8-(2-(hydroxymethyl)thieno[3,2-b]pyridin-7-yl)-6-(trifluoromethyl)-3,4-dihydroquinolin-1(2H)-yl)-5-azaspiro[3.4]octane-5-carboxylate and succinimide, following the procedure described in the synthesis of Example 78. The product was purified by preparative HPLC. ¹H NMR (400 MHz, CD₃OD) δ=8.73 (d, J=4.8 Hz, 1H), 8.53 (s, 1H), 7.56-7.46 (m, 2H), 7.40 (d, J=13.6 Hz, 2H), 4.96 (s, 2H), 3.67 (s, 1H), 2.94 (d, J=6.4 Hz, 2H), 2.75 (s, 4H), 2.48 (s, 1H), 2.21-1.87 (m, 5H), 1.80-1.35 (m, 6H), 1.31-1.18 (m, 2H). LCMS R_{T}=1.426 min, m/z=555.4 [M+H]⁺

### Example 164a and 164b: (S)-8-(2-((4-methyl-6-oxopyridazin-1(6H)-yl)methyl)thieno[3,2-b]pyridin-7-yl)-1-(5-azaspiro[3.4]octan-7-yl)-1,2,3,4-tetrahydroquinoline-6-carbonitrile, formic acid salt and (R)-8-(2-((4-methyl-6-oxopyridazin-1(6H)-yl)methyl)thieno[3,2-b]pyridin-7-yl)-1-(5-azaspiro[3.4]octan-7-yl)-1,2,3,4-tetrahydroquinoline-6-carbonitrile, formic acid salt

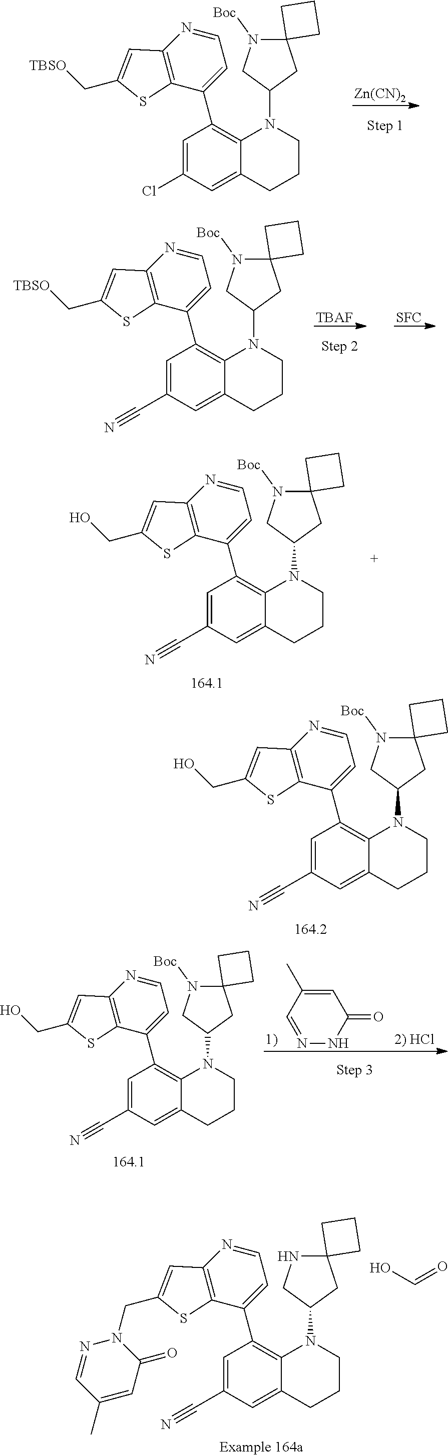

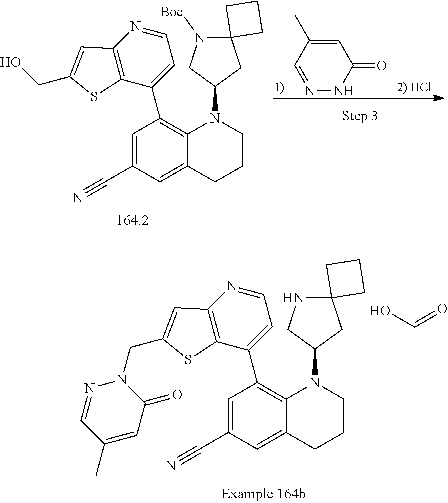

### Step 1: tert-butyl 7-(8-(2-(((tert-butyldimethylsilyl)oxy)methyl)thieno[3,2-b]pyridin-7-yl)-6-chloro-3,4-dihydroquinolin-1(2H)-yl)-5-azaspiro[3.4]octane-5-carboxylate

To a solution of tert-butyl 7-[8-[2-[[tert-butyl(dimethyl)silyl]oxymethyl]thieno[3,2-b]pyridin-7-yl]-6-chloro-3,4-dihydro-2H-quinolin-1-yl]-5-azaspiro[3.4]octane-5-carboxylate (1.2 g, 1.83 mmol) in dioxane (10 mL) was added cyanide zinc (430 mg, 3.67 mmol), N, N-diisopropylethylamine (0.9 mL, 5.50 mmol) and bis (tri-tert-butylphosphine) palladium (280 mg, 0.55 mmol) at 25° C. After stirred at 90° C. for 12 h, the reaction mixture was diluted with water (10 mL) and extracted with ethyl acetate (5 mL×4). The combined organic layers were dried over anhydrous sodium sulfate, filtered and the filtrate was concentrated under reduced pressure. The residue was purified by column chromatography to afford the title compound. LCMS R_{T}=0.660, m/z=645.4 [M+H]⁺.

### Step 2: (S)-tert-butyl 7-(6-cyano-8-(2-(hydroxymethyl)thieno[3,2-b]pyridin-7-yl)-3,4-dihydroquinolin-1(2H)-yl)-5-azaspiro[3.4]octane-5-carboxylate and (R)-tert-butyl 7-(6-cyano-8-(2-(hydroxymethyl)thieno[3,2-b]pyridin-7-yl)-3,4-dihydroquinolin-1(2H)-yl)-5-azaspiro[3.4]octane-5-carboxylate

To a solution of tert-butyl 7-[8-[2-[[tert-butyl(dimethyl)silyl]oxymethyl]thieno[3,2-b]pyridin-7-yl]-6-cyano-3,4-dihydro-2H-quinolin-1-yl]-5-azaspiro[3.4]octane-5-carboxylate (540 mg, 0.83 mmol) in THF (3 mL) was added tetrabutylammonium fluoride (0.8 mL, 1 M in THF) at 25° C. After stirred at 25° C. for 0.5 h, the reaction mixture was concentrated under reduced pressure. The residue was purified by column chromatography. The racemate was separated by SFC to give first eluting fraction (164.1, Rₜ=3.340 min; LCMS R_{T}=0.543 min, m/z=531.3 [M+H]+) and second eluting fraction (164.2, R₁=4.026 min; LCMS R_{T}=0.533 min, m/z=531.7 [M+H]+).

### Step 3

Example 164a was prepared from 4-methyl-1H-pyridazin-6-one and 164.1, following the procedure described in the synthesis of Example 108. The product was purified by preparative HPLC to afford the title compound. ¹H NMR (400 MHz, CD₃OD) δ=8.73 (d, J=3.6 Hz, 1H), 8.51 (s, 1H), 7.87 (d, J=2.0 Hz, 1H), 7.65-7.54 (m, 1H), 7.51-7.41 (m, 3H), 6.79 (dd, J=1.8 Hz, 1.2 Hz, 1H), 5.67-5.56 (m, 2H), 3.83-3.54 (m, 1H), 3.30-3.12 (m, 2H), 2.94-2.85 (m, 2H), 2.53-2.39 (m, 1H), 2.20-2.08 (m, 4H), 2.04-1.71 (m, 7H), 1.56-1.31 (m, 2H), 0.92-0.70 (m, 1H). LCMS R_{T}=0.439 min, m/z=523.2 [M+H]⁺.

Example 164b was prepared from 164.2 and 4-methyl-1H-pyridazin-6-one, following the procedure described in the synthesis of Example 108. The product was purified by preparative HPLC. ¹H NMR (400 MHz, CD₃OD) δ=8.73 (s, 1H), 8.51 (s, 1H), 7.87 (d, J=2.0 Hz, 1H), 7.63-7.42 (m, 4H), 6.79 (s, 1H), 5.66-5.53 (m, 2H), 3.80-3.58 (m, 1H), 3.27-2.80 (m, 4H), 2.56-2.35 (m, 1H), 2.25 (s, 3H), 2.21-1.23 (m, 10H), 0.91-0.66 (m, 1H). LCMS R_{T}=1.993, m/z=523.2 [M+H]⁺.

The absolute configuration was tentatively assigned based on the relative potency of the pair diastereomers in biology assays.

### Example 165a, 165b, 165c and 165d: 1-((7-((1aR,7bS)-6-chloro-3-((S)-5-azaspiro[3.4]octan-7-yl)-1a,2,3,7b-tetrahydro-1H-cyclopropa[c]quinolin-4-yl)thieno[3,2-b]pyridin-2-yl)methyl)pyrrolidine-2,5-dione, hydrochloride 1-((7-((1aR,7bS)-6-chloro-3-((R)-5-azaspiro[3.4]octan-7-yl)-1a,2,3,7b-tetrahydro-1H-cyclopropa[c]quinolin-4-yl)thieno[3,2-b]pyridin-2-yl)methyl)pyrrolidine-2,5-dione, hydrochloride 1-((7-((1aS,7bR)-6-chloro-3-((S)-5-azaspiro[3.4]octan-7-yl)-1a,2,3,7b-tetrahydro-1H-cyclopropa[c]quinolin-4-yl)thieno[3,2-b]pyridin-2-yl)methyl)pyrrolidine-2,5-dione, formic acid salt 1-((7-((1aS,7bR)-6-chloro-3-((R)-5-azaspiro[3.4]octan-7-yl)-1a,2,3,7b-tetrahydro-1H-cyclopropa[c]quinolin-4-yl)thieno[3,2-b]pyridin-2-yl)methyl)pyrrolidine-2,5-dione, formic acid salt

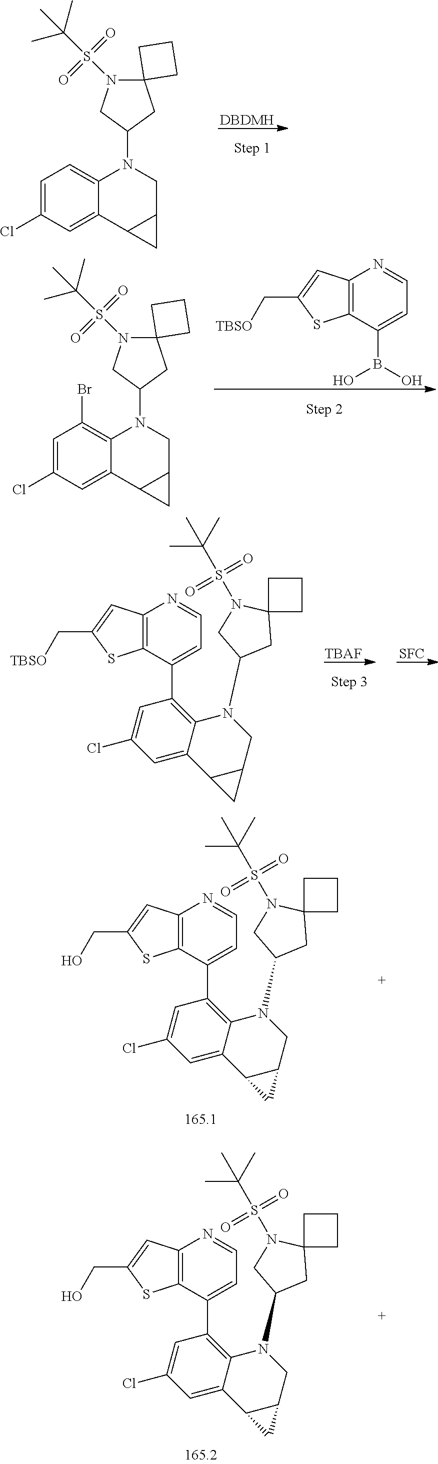

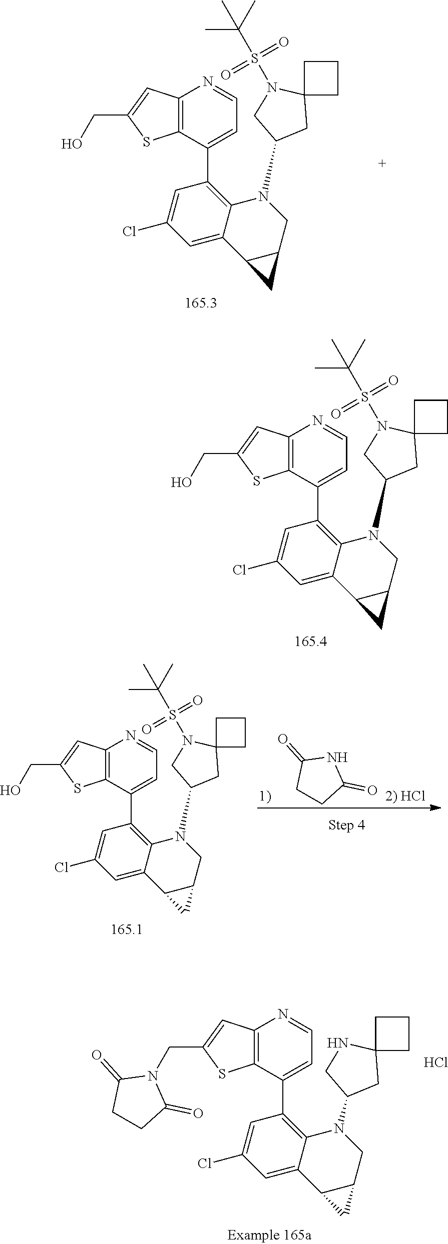

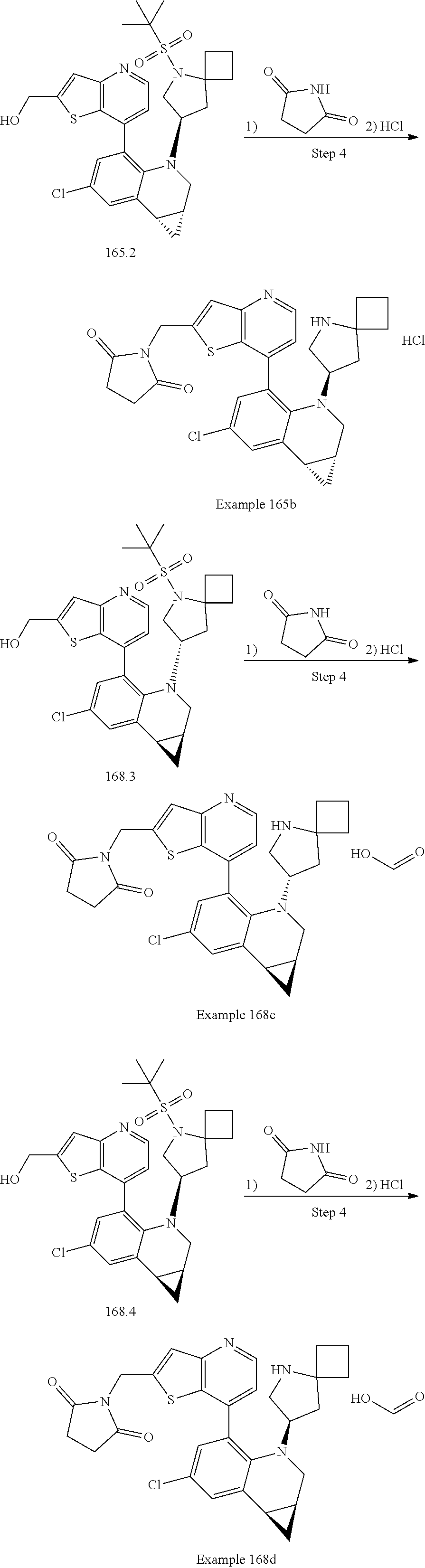

### Step 1: 4-bromo-3-(5-(tert-butylsulfonyl)-5-azaspiro[3.4]octan-7-yl)-6-chloro-1a,2,3,7b-tetrahydro-1H-cyclopropa[c]quinoline

To a solution of 3-(5-(tert-butylsulfonyl)-5-azaspiro[3.4]octan-7-yl)-6-chloro-1a,2,3,7b-tetrahydro-1H-cyclopropa[c]quinoline (540 mg, 1.32 mmol) in acetic acid (7 mL) was added 1,3-dibromo-5,5-dimethylimidazolidine-2,4-dione (188 mg, 0.66 mmol). After stirred at 25° C. at 1 h, the mixture was concentrated. Then the residue was diluted with water (20 mL) and extracted with ethyl acetate (15 mL×2). The combined organic layers were washed with brine (20 mL), dried over anhydrous sodium sulfate and concentrated under reduced pressure. The residue was purified by column chromatography to afford the title compound. LCMS R_{T}=1.692 min, m/z=487.2, 489.2 [M+H]⁺.

### Step 2: 2-(((tert-butyldimethylsilyl)oxy)methyl)-7-(3-(5-(tert-butylsulfonyl)-5-azaspiro[3.4]octan-7-yl)-6-chloro-1a,2,3,7b-tetrahydro-1H-cyclopropa[c]quinolin-4-yl)thieno[3,2-b]pyridine

To a solution of (2-(((tert-butyldimethylsilyl)oxy)methyl)thieno[3,2-b]pyridin-7-yl)boronic acid (244 mg, 0.75 mmol), 4-bromo-3-(5-(tert-butylsulfonyl)-5-azaspiro[3.4]octan-7-yl)-6-chloro-1a,2,3,7b-tetrahydro-1H-cyclopropa[c]quinoline (230 mg, 0.47 mmol) in dioxane (5 mL) and water (0.4 mL) was added potassium carbonate (163 mg, 1.18 mmol) and dichloro[1,1′-bis(diphenylphosphino)ferrocene]palladium(II) (69 mg, 0.09 mmol). After stirred at 100° C. for 2 h under nitrogen atmosphere, the mixture was concentrated. The residue was purified by column chromatography to afford the title compound. LCMS R_{T}=3.083 min, m/z=686.4 [M+H]⁺.

### Step 3

To a solution of 2-(((tert-butyldimethylsilyl)oxy)methyl)-7-(3-(5-(tert-butylsulfonyl)-5-azaspiro[3.4]octan-7-yl)-6-chloro-1a,2,3,7b-tetrahydro-1H-cyclopropa[c]quinolin-4-yl)thieno[3,2-b]pyridine (330 mg, 0.48 mmol) in THF (5 mL) was added tetrabutylammonium fluoride (1 M in THF, 0.53 mL). After stirred at 25° C. for 0.5 h, the mixture was concentrated. The residue was purified by column chromatography. The racemate was separated by SFC to afford first eluting fraction (165.1, Rt=3.267 min), second eluting fraction (165.2, Rt=3.600 min), third eluting fraction (165.3, Rt=3.960 min) and fourth eluting fraction (165.4, Rt=4.492 min). LCMS R_{T}=1.917 min, m/z=572.4 [M+H]⁺.

### Step 4

Example 165a was prepared from succinimide and 165.1, following the procedure described in the synthesis of Example 108. The product was purified by preparative HPLC. ¹H NMR (400 MHz, CD₃OD) δ=8.84 (d, J=4.8 Hz, 1H), 7.66 (s, 2H), 7.43 (d, J=2.0 Hz, 1H), 7.13 (s, 1H), 5.02 (s, 2H), 3.62 (d, J=8.0 Hz, 1H), 3.44-3.33 (m, 2H), 3.19 (d, J=14.0 Hz, 1H), 3.01-2.83 (m, 2H), 2.78 (s, 4H), 2.35-2.25 (m, 1H), 2.13-1.95 (m, 4H), 1.82-1.73 (m, 1H), 1.50 (d, J=8.8 Hz, 2H), 1.21-1.11 (m, 1H), 1.02 (s, 1H), 0.74 (br s, 1H). LCMS R_{T}=1.360 min, m/z=533.3 [M+H]⁺.

Example 165b was prepared from succinimide, 165.2, following the procedure described in the synthesis of Example 108. The product was purified by preparative HPLC. ¹H NMR (400 MHz, CD₃OD) δ=8.78 (s, 1H), 7.58 (s, 2H), 7.39 (d, J=2.4 Hz, 1H), 7.07 (d, J=2.4 Hz, 1H), 4.99 (s, 2H), 3.69 (s, 1H), 3.35 (s, 1H), 3.29-3.19 (m, 2H), 2.77 (s, 4H), 2.37-2.27 (m, 1H), 2.17 (dd, J=4.4, 8.0 Hz, 1H), 2.11-1.95 (m, 3H), 1.91-1.44 (m, 6H), 1.15 (dt, J=4.8, 8.4 Hz, 1H), 0.90 (d, J=4.0 Hz, 1H). LCMS R_{T}=1.450 min, m/z=533.4 [M+H]⁺.

Example 165c was prepared from 165.3 and succinimide, following the procedure described in the synthesis of Example 108. The product was purified by preparative HPLC. ¹H NMR (400 MHz, CD₃OD) δ=8.71 (d, J=4.8 Hz, 1H), 8.54 (s, 1H), 7.60 (d, J=8.0 Hz, 1H), 7.48-7.32 (m, 2H), 7.16-7.02 (m, 1H), 5.17-5.01 (m, 2H), 4.87-4.51 (m, 2H), 3.62 (s, 1H), 3.54-3.36 (m, 2H), 3.25-2.67 (m, 4H), 2.23-1.80 (m, 5H), 1.80-1.20 (m, 5H), 1.12-0.93 (m, 1H), 0.89-0.81 (m, 1H). LCMS R_{T}=1.449 min, m/z=533.3 [M+H]+

Example 165d was prepared from 165.4 (40 mg, 0.07 mmol) and succinimide (21 mg, 0.21 mmol), following the procedure described in the synthesis of Example 108. The product was purified by preparative HPLC. ¹H NMR (400 MHz, CD₃OD) δ=8.71 (d, J=4.8 Hz, 1H), 8.51 (s, 1H), 7.54 (s, 1H), 7.45-7.28 (m, 2H), 7.03 (d, J=2.4 Hz, 1H), 5.00-4.94 (m, 2H), 4.86-4.52 (m, 2H), 3.61 (s, 1H), 3.24-3.13 (m, 1H), 3.02-2.69 (m, 6H), 2.28-2.12 (m, 1H), 2.10-1.75 (m, 4H), 1.74-1.61 (m, 1H), 1.50-1.16 (m, 2H), 1.09 (dt, J=4.9, 8.2 Hz, 1H), 1.00-0.65 (m, 2H). LCMS R_{T}=1.352 min, m/z=533.4 [M+H]⁺

The absolute configuration was tentatively assigned based on the relative potency of the pair diastereomers in biology assays.

### Example 166: 2-((7-((1aS,7bR)-6-chloro-3-((S)-5-azaspiro[3.4]octan-7-yl)-1a,2,3,7b-tetrahydro-1H-cyclopropa[c]quinolin-4-yl)thieno[3,2-b]pyridin-2-yl)methyl)-5-methylpyridazin-3(2H)-one

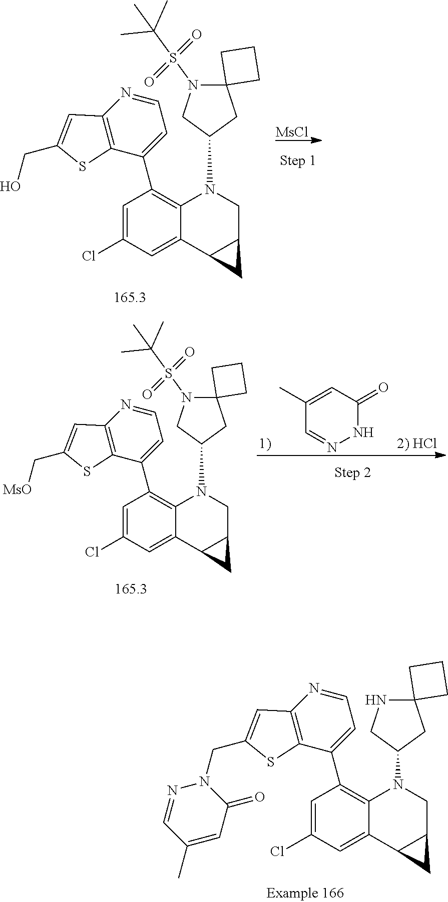

### Step 1: (7-((1aS,7bR)-3-((S)-5-(tert-butylsulfonyl)-5-azaspiro[3.4]octan-7-yl)-6-chloro-1a,2,3,7b-tetrahydro-1H-cyclopropa[c]quinolin-4-yl)thieno[3,2-b]pyridin-2-yl)methyl methanesulfonate

To a solution of 165.3 (30 mg, 0.05 mol) in dichloromethane (5 mL) was added triethylamine (16 mg, 0.16 mmol) and methane sulfonyl chloride (18 mg, 0.16 mmol) at 0° C. After stirred at 25° C. for 1 h, the reaction mixture was quenched by addition of saturated aqueous sodium bicarbonate (10 mL), and then extracted with dichloromethane (10 mL×3). The combined organic layers were dried over sodium sulphate, filtered and the filtrate was concentrated under reduced pressure to afford the title compound which was used in next step without further purification. LCMS R_{T}=0.628 min, m/z=650.1 [M+H]⁺.

### Step 2

To a solution of [7-[(1aS,7bR)-3-[(7R)-5-tert-butylsulfonyl-5-azaspiro[3.4]octan-7-yl]-6-chloro-1,1a,2,7b-tetrahydrocyclopropa[c]quinolin-4-yl]thieno[3,2-b]pyridin-2-yl]methyl methanesulfonate (30 mg, 0.05 mmol) in DMF (3 mL) was added potassium iodide (8 mg, 0.05 mmol) and potassium carbonate (13 mg, 0.09 mmol) and 4-methyl-1H-pyridazin-6-one (8 mg, 0.07 mmol) at 25° C. After stirred at 25° C. for 4 h, the reaction mixture was concentrated under reduced pressure to remove the solvent. The residue was added hydrochloric acid (12 M, 4 mL). After stirred at 25° C. for 30 min, the reaction mixture was concentrated under reduced pressure. The residue was purified by preparative HPLC to afford Example 166. ¹H NMR (400 MHz, CD30D) 6=8.71 (s, 1H), 7.87 (d, J=2.0 Hz, 1H), 7.58 (s, 1H), 7.35 (d, J=2.4 Hz, 2H), 7.02 (d, J=2.4 Hz, 1H), 6.79 (s, 1H), 5.60 (s, 2H), 3.87-3.45 (m, 1H), 3.26-3.03 (m, 2H), 2.25 (s, 4H), 2.17-1.89 (m, 5H), 1.77 (s, 3H), 1.82-1.69 (m, 1H), 1.12 (d, J=4.8, 8.4 Hz, 2H), 0.89 (d, J=4.0 Hz, 1H). LCMS: RT=0.505 min m/z=544.2 [M+H]⁺.

### Example 167a, 167b, 167c and 167d: 1-((4-((1aS,7bR)-6-chloro-3-((S)-5-azaspiro[3.4]octan-7-yl)-1a,2,3,7b-tetrahydro-1H-cyclopropa[c]quinolin-4-yl)thieno[3,2-d]pyrimidin-6-yl)methyl)pyrrolidine-2,5-dione, hydrochloride acid salt 1-((4-((1aS,7bR)-6-chloro-3-((R)-5-azaspiro[3.4]octan-7-yl)-1a,2,3,7b-tetrahydro-1H-cyclopropa[c]quinolin-4-yl)thieno[3,2-d]pyrimidin-6-yl)methyl)pyrrolidine-2,5-dione, hydrochloride acid salt 1-((4-((1aR,7bS)-6-chloro-3-((R)-5-azaspiro[3.4]octan-7-yl)-1a,2,3,7b-tetrahydro-1H-cyclopropa[c]quinolin-4-yl)thieno[3,2-d]pyrimidin-6-yl)methyl)pyrrolidine-2,5-dione, hydrochloride acid salt and 1-((4-((1aR,7bS)-6-chloro-3-((S)-5-azaspiro[3.4]octan-7-yl)-1a,2,3,7b-tetrahydro-1H-cyclopropa[c]quinolin-4-yl)thieno[3,2-d]pyrimidin-6-yl)methyl)pyrrolidine-2,5-dione, hydrochloride acid salt

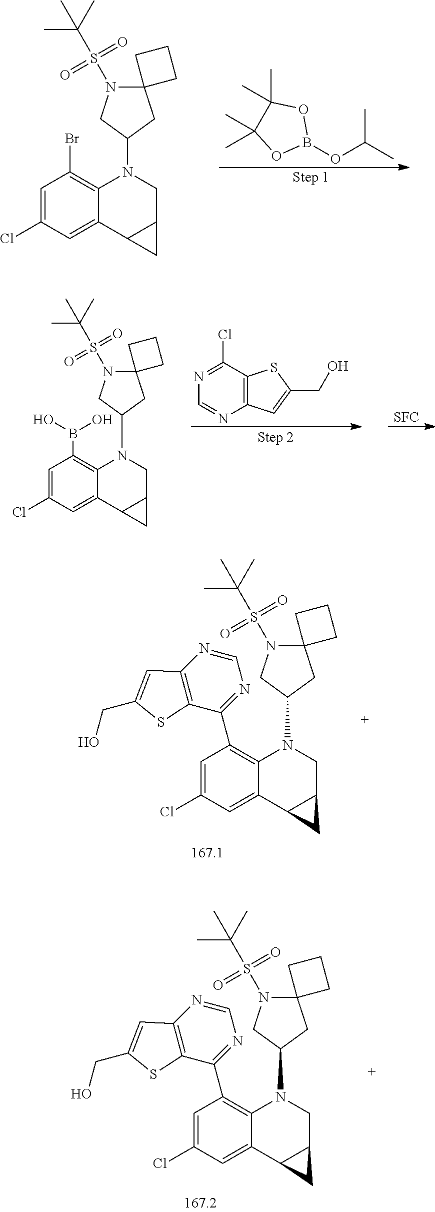

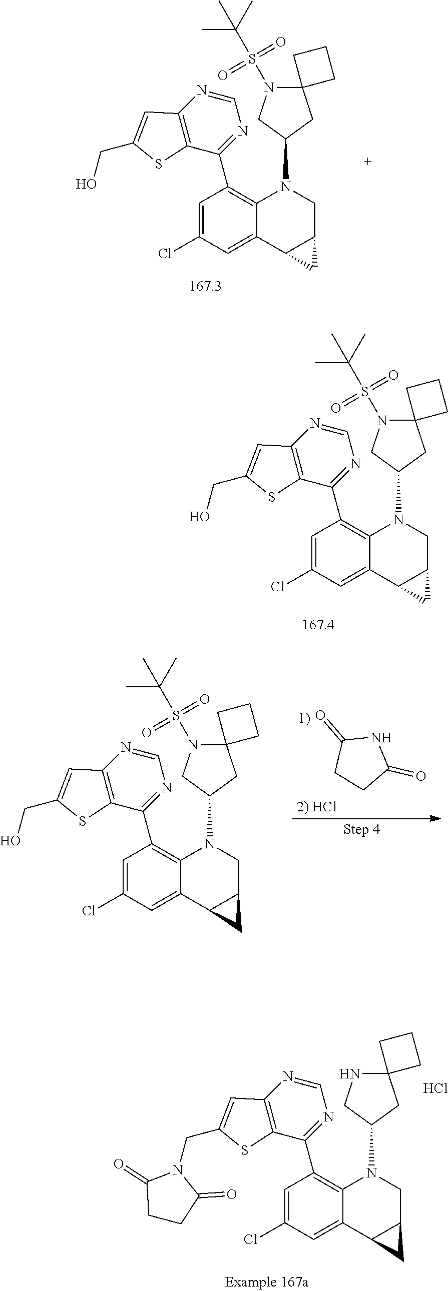 [SMILES: CC(C)(C)S(=O)(=O)N1C[C@H](N2C[C@@H]3C[C@@H]3c3cc(Cl)cc(-c4ncnc5cc(CO)sc45)c32)CC12CCC2 ; CC(C)(C)S(=O)(=O)N1C[C@@H](N2C[C@@H]3C[C@@H]3c3cc(Cl)cc(-c4ncnc5cc(CO)sc45)c32)CC12CCC2 ; CC(C)(C)S(=O)(=O)N1C[C@@H](N2C[C@H]3C[C@H]3c3cc(Cl)cc(-c4ncnc5cc(CO)sc45)c32)CC12CCC2 ; O=C1CCC(=O)N1 ; O=C1CCC(=O)N1Cc1cc2ncnc(-c3cc(Cl)cc4c3N([C@@H]3CNC5(CCC5)C3)C[C@H]3C[C@@H]43)c2s1 ; Cl]

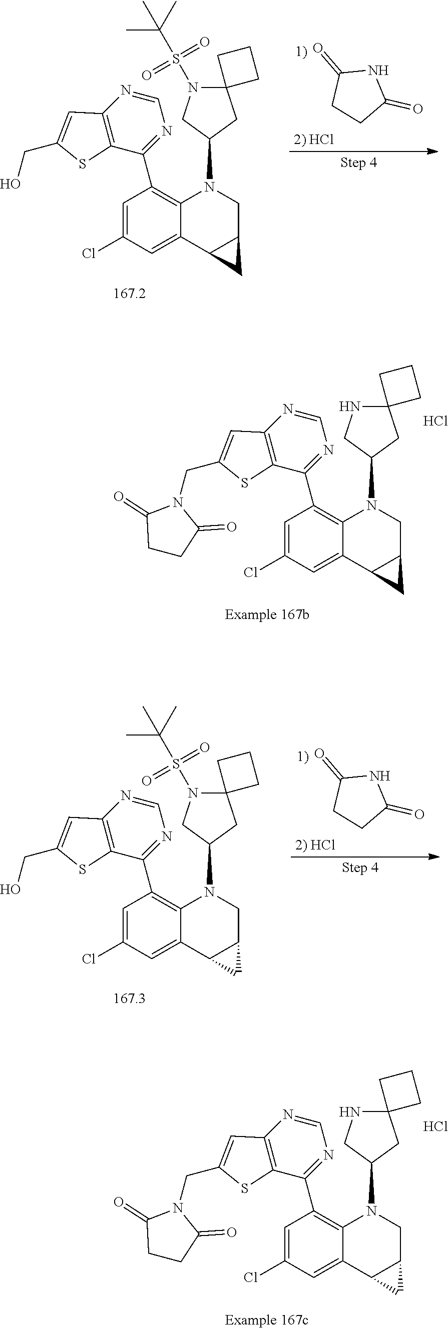

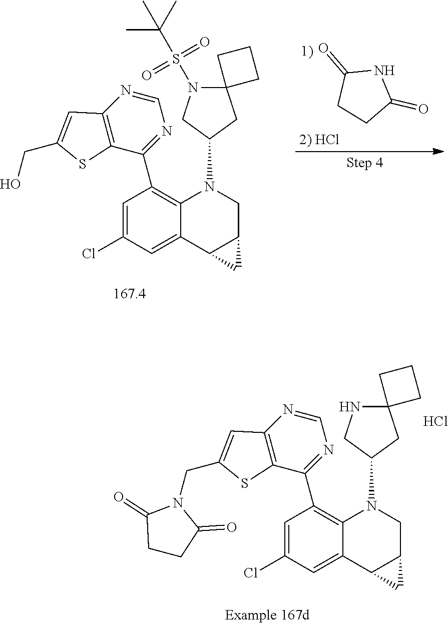 [SMILES: CC(C)(C)S(=O)(=O)N1C[C@@H](N2C[C@@H]3C[C@@H]3c3cc(Cl)cc(-c4ncnc5cc(CO)sc45)c32)CC12CCC2 ; O=C1CCC(=O)N1 ; O=C1CCC(=O)N1Cc1cc2ncnc(-c3cc(Cl)cc4c3N([C@@H]3CNC5(CCC5)C3)C[C@@H]3C[C@H]43)c2s1 ; Cl]

### Step 1: (3-(5-(tert-butylsulfonyl)-5-azaspiro[3.4]octan-7-yl)-6-chloro-1a,2,3,7b-tetrahydro-1H-cyclopropa[c]quinolin-4-yl)boronic acid

To a solution of isopropylmagnesium lithium chloride (7 mL, 1.3 M in THF) was added 4-bromo-3-(5-tert-butylsulfonyl-5-azaspiro[3.4]octan-7-yl)-6-chloro-1,1a,2,7b-tetrahydrocyclopropa[c]quinoline (700 mg, 1.43 mmol) in THF (10 mL) at 0° C. After stirred for 0.5 h, the mixture was added 2-isopropoxy-4,4,5,5-tetramethyl-1,3,2-dioxaborolane (800 mg, 4.30 mmol) at 0° C. After stirred at 0° C. for 0.5 h, the mixture was quenched by addition of saturated aqueous ammonium chloride (10 mL), then extracted with ethyl acetate (10 mL×3). The organic layers were dried over anhydrous sodium sulfate, filtered and the filtrate was concentrated under reduced pressure to afford the title compound which was used in next step without further purification. LCMS R_{T}=0.557 min, m/z=453.2 [M+H]⁺.

### Step 2

To a solution of [3-(5-tert-butylsulfonyl-5-azaspiro[3.4]octan-7-yl)-6-chloro-1,1a,2,7b-tetrahydrocyclopropa[c]quinolin-4-yl]boronic acid (649 mg, 1.43 mmol) in dioxane (20 mL) and water (2 mL) was added potassium carbonate (594 mg, 4.30 mmol), (4-chlorothieno[3,2-d]pyrimidin-6-yl)methanol (431 mg, 2.15 mmol) and palladium dichloride[1,1′-bis (diphenylphosphine) ferrocene](104 mg, 0.14 mmol) at 25° C. After stirred at 100° C. for 3 h, the reaction mixture was concentrated under reduced pressure. The residue was purified by column chromatography. The racemate was separated by SFC to give first eluting fraction (167.1, Rt=3.494 min; LCMS R_{T}=1.043 min, m/z=573.0 [M+H]+), second eluting fraction (167.2, Rt=4.017 min; LCMS R_{T}=1.043 min, m/z=573.0 [M+H]+), third eluting fraction (167.3, Rt=4.375 min; LCMS R_{T}=1.020 min, m/z=573.0 [M+H]+) and forth eluting fraction (167.4, Rt=4.677 min; LCMS R_{T}=1.023 min, m/z=573.0 [M+H]+).

### Step 4

Example 167a was prepared from 167.1 and succinimide, following the procedure described in the synthesis of Example 108. The product was purified by preparative HPLC. ¹H NMR (400 MHz, CD₃OD) δ=9.35-9.25 (m, 1H), 7.68-7.59 (m, 1H), 7.49-7.40 (m, 1H), 7.25-7.13 (m, 1H), 5.06-5.02 (m, 2H), 3.69-3.56 (m, 1H), 3.40-3.32 (m, 2H), 2.85-2.75 (m, 4H), 2.55-2.28 (m, 2H), 2.25-1.95 (m, 5H), 1.94-1.74 (m, 3H), 1.73-1.60 (m, 1H), 1.58-1.40 (m, 1H), 1.23-1.13 (m, 1H), 0.92-0.83 (m, 1H). LCMS R_{T}=1.362 min, m/z=534.3 [M+H]+

Example 167b was prepared from 167.2 and succinimide, following the procedure described in the synthesis of Example 108. The product was purified by preparative HPLC. ¹H NMR (400 MHz, CD₃OD) δ=9.34-9.10 (m, 1H), 7.54 (s, 1H), 7.37 (d, J=2.4 Hz, 1H), 7.13 (d, J=2.4 Hz, 1H), 5.01 (s, 2H), 3.55-3.42 (m, 1H), 3.32-3.19 (m, 2H), 2.78 (s, 4H), 2.09-1.91 (m, 5H), 1.80-1.35 (m, 7H), 1.17-1.07 (m, 1H), 0.99-0.91 (m, 1H). LCMS R_{T}=1.400 min, m/z=534.4 [M+H]⁺.

Example 167c was prepared from 167.3 and succinimide, following the procedure described in the synthesis of Example 108. The product was purified by preparative HPLC. ¹H NMR (400 MHz, CD₃OD) δ=9.26 (s, 1H), 7.62 (s, 1H), 7.43 (d, J=2.4 Hz, 1H), 7.20 (d, J=2.4 Hz, 1H), 5.04 (s, 2H), 3.62-3.52 (m, 1H), 3.43-3.35 (m, 1H), 3.24 (d, J=13.2 Hz, 1H), 3.03-2.86 (m, 2H), 2.80 (s, 4H), 2.37-2.27 (m, 1H), 2.14-1.99 (m, 4H), 1.83-1.76 (m, 1H), 1.59-1.35 (m, 3H), 1.15 (dt, J=4.8, 8.0 Hz, 1H), 1.10-1.00 (m, 1H), 0.74 (q, J=4.8 Hz, 1H). LCMS R_{T}=2.028 min, m/z=534.2 [M+H]⁺.

Example 167d was prepared from 167.4 and succinimide, following the procedure described in the synthesis of Example 108. The product was purified by preparative HPLC. ¹H NMR (400 MHz, CD₃OD) δ=9.26 (s, 1H), 7.62 (s, 1H), 7.43 (d, J=2.4 Hz, 1H), 7.20 (d, J=2.4 Hz, 1H), 5.04 (s, 2H), 3.62-3.52 (m, 1H), 3.43-3.35 (m, 1H), 3.24 (d, J=13.2 Hz, 1H), 3.03-2.86 (m, 2H), 2.80 (s, 4H), 2.37-2.27 (m, 1H), 2.14-1.99 (m, 4H), 1.83-1.76 (m, 1H), 1.59-1.35 (m, 3H), 1.15 (dt, J=8.0 Hz, 4.8 Hz, 1H), 1.10-1.00 (m, 1H), 0.74 (q, J=4.8 Hz, 1H). LCMS R_{T}=1.280 min, m/z=534.3 [M+H]⁺.

The absolute configuration was tentatively assigned based on the relative potency of the pair diastereomers in biology assays.

### Example 168: (1aS,7bR)-4-(2-((2,5-dioxopyrrolidin-1-yl)methyl)thieno[3,2-b]pyridin-7-yl)-3-((S)-5-azaspiro[3.4]octan-7-yl)-1a,2,3,7b-tetrahydro-1H-cyclopropa[c]quinoline-6-carbonitrile, formic acid salt

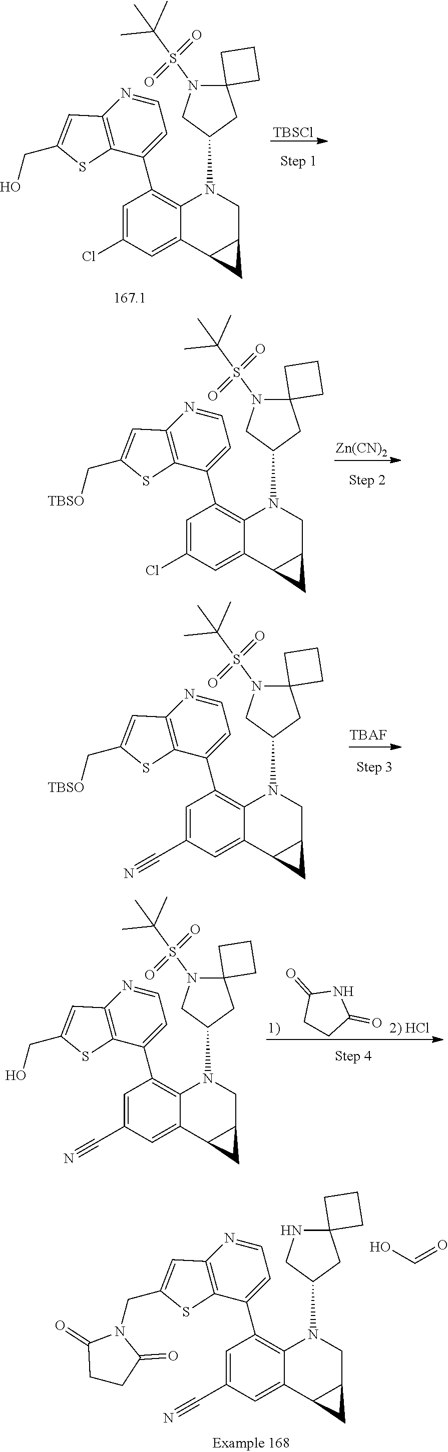

### Step 1: 2-(((tert-butyldimethylsilyl)oxy)methyl)-7-((1aS,7bR)-3-((S)-5-(tert-butylsulfonyl)-5-azaspiro[3.4]octan-7-yl)-6-chloro-1a,2,3,7b-tetrahydro-1H-cyclopropa[c]quinolin-4-yl)thieno[3,2-b]pyridine

To a solution of 167.1 (250 mg, 0.44 mmol) in dichloromethane (5 mL) was added imidazole (59 mg, 0.87 mmol) and tert-butyldimethylsilyl chloride (132 mg, 0.87 mmol) at 0° C. After stirred for 30 min at 25° C., the reaction mixture was concentrated under reduced pressure. The residue was purified by column chromatography to afford the title compound. LCMS R_{T}=0.683 min, m/z=686.6 [M+H]⁺.

### Step 2: (1aS,7bR)-4-(2-(((tert-butyldimethylsilyl)oxy)methyl)thieno[3,2-b]pyridin-7-yl)-3-((S)-5-(tert-butylsulfonyl)-5-azaspiro[3.4]octan-7-yl)-1a,2,3,7b-tetrahydro-1H-cyclopropa[c]quinoline-6-carbonitrile

To a solution of 2-(((tert-butyldimethylsilyl)oxy)methyl)-7-((1aS,7bR)-3-((S)-5-(tert-butylsulfonyl)-5-azaspiro[3.4]octan-7-yl)-6-chloro-1a,2,3,7b-tetrahydro-1H-cyclopropa[c]quinolin-4-yl)thieno[3,2-b]pyridine (250 mg, 0.36 mmol) and in dioxane (3 mL) was added zinc cyanide (64 mg, 0.55 mmol) and bis(tri-tert-butylphosphine)palladium (37 mg, 0.07 mmol) and diisopropylethylamine (118 mg, 0.91 mmol) at 25° C. After stirred at 90° C. for 12 h, the reaction mixture was concentrated under reduced pressure to remove the solvent. The residue was purified by preparative TLC to afford the title compound. LCMS R_{T}=2.084 min, m/z=677.3 [M+H]⁺.

### Step 3: (1aS,7bR)-3-((S)-5-(tert-butylsulfonyl)-5-azaspiro[3.4]octan-7-yl)-4-(2-(hydroxymethyl)thieno[3,2-b]pyridin-7-yl)-1a,2,3,7b-tetrahydro-1H-cyclopropa[c]quinoline-6-carbonitrile

To a solution of (1aS,7bR)-4-(2-(((tert-butyldimethylsilyl)oxy)methyl)thieno[3,2-b]pyridin-7-yl)-3-((S)-5-(tert-butylsulfonyl)-5-azaspiro[3.4]octan-7-yl)-1a,2,3,7b-tetrahydro-1H-cyclopropa[c]quinoline-6-carbonitrile (240 mg, 0.35 mmol) in THF (5 mL) was added tetrabutyl ammonium fluoride (1 M, 0.53 mL) at 25° C. After stirred at 25° C. for 1 h, the reaction mixture was concentrated under reduced pressure to remove the solvent. The residue was purified by pre-TLC to afford the title compound. LCMS R_{T}=0.532 min, m/z=563.2 [M+H]⁺.

### Step 4

Example 168 was prepared from (1aS,7bR)-3-((S)-5-(tert-butylsulfonyl)-5-azaspiro[3.4]octan-7-yl)-4-(2-(hydroxymethyl)thieno[3,2-b]pyridin-7-yl)-1a,2,3,7b-tetrahydro-1H-cyclopropa[c]quinoline-6-carbonitrile and succinimide, following the procedure described in the synthesis of Example 108. The product was purified by preparative HPLC. ¹H NMR (400 MHz, CD₃OD) δ=8.73-8.73 (m, 1H), 7.64 (d, J=2.0 Hz, 1H), 7.50 (s, 2H), 7.36-7.35 (m, 1H), 7.36 (s, 1H), 4.94 (s, 2H), 3.85-3.76 (m, 1H), 3.36 (s, 1H), 3.25-3.12 (m, 1H), 2.74 (s, 4H), 2.17-1.91 (m, 6H), 1.68 (m, 5H), 1.13 (dt, J=4.8, 8.4 Hz, 1H), 1.02-0.94 (m, 2H). LCMS R_{T}=1.477 min, m/z=524.3 [M+H]⁺.

### Example 169: (1aS,7bR)-4-(2-((2,6-dioxo-3-(2,2,2-trifluoroethyl)-3,6-dihydropyrimidin-1(2H)-yl)methyl)thieno[3,2-b]pyridin-7-yl)-3-((S)-5-azaspiro[3.4]octan-7-yl)-1a,2,3,7b-tetrahydro-1H-cyclopropa[c]quinoline-6-carbonitrile

Prepared from (1aS,7bR)-3-((S)-5-(tert-butylsulfonyl)-5-azaspiro[3.4]octan-7-yl)-4-(2-(hydroxymethyl)thieno[3,2-b]pyridin-7-yl)-1a,2,3,7b-tetrahydro-1H-cyclopropa[c]quinoline-6-carbonitrile in toluene (1 mL) and 1-(2,2,2-trifluoroethyl)pyrimidine-2,4-dione, following the procedure described in the synthesis of Example 108. The product was purified by preparative HPLC. ¹H NMR (400 MHz, CD₃OD) δ=8.75-8.56 (m, 1H), 7.68-7.41 (m, 4H), 7.32 (d, J=2.0 Hz, 1H), 5.86 (d, J=8.0 Hz, 1H), 5.44-5.28 (m, 2H), 4.61 (s, 2H), 3.79-3.42 (m, 1H), 3.29-3.21 (m, 1H), 2.13-1.66 (m, 6H), 1.62-1.41 (m, 5H), 1.09 (dt, J=8.4 Hz, 5.2 Hz, 1H), 1.35-1.03 (m, 1H), 1.02-0.88 (m, 2H). LCMS RT=1.633 min, m/z=619.4 [M+H]⁺.

### Example 170: (1aS,7bR)-4-(2-((2,6-dioxo-3-(2,2,2-trifluoroethyl)-3,6-dihydropyrimidin-1(2H)-yl)methyl)thieno[3,2-b]pyridin-7-yl)-3-((S)-5-azaspiro[3.4]octan-7-yl)-1a,2,3,7b-tetrahydro-1H-cyclopropa[c]quinoline-6-carboxamide

Example 170 was isolated as a side product in the synthesis of Example 169. ¹H NMR (400 MHz, DMSO) δ=8.90-8.56 (m, 1H), 7.95-7.68 (m, 3H), 7.62-7.29 (m, 2H), 7.29-7.02 (m, 1H), 7.11 (br s, 1H), 5.89 (br, 1H), 5.41-5.18 (m, 2H), 4.81-4.58 (m, 2H), 3.22-3.04 (m, 3H), 2.13-1.67 (m, 6H), 1.64-1.30 (m, 5H), 1.26-0.96 (m, 2H), 0.93-0.71 (m, 2H). LCMS R_{T}=0.418 min, m/z=637.2 [M+H]⁺

### Example 171a and 171b: (R)-1-((7-(7-chloro-4-(5-azaspiro[3.4]octan-7-yl)-3,4-dihydro-2H-benzo[b][1,4]oxazin-5-yl)thieno[3,2-b]pyridin-2-yl)methyl)pyrrolidine-2,5-dione and (S)-1-((7-(7-chloro-4-(5-azaspiro[3.4]octan-7-yl)-3,4-dihydro-2H-benzo[b][1,4]oxazin-5-yl)thieno[3,2-b]pyridin-2-yl)methyl)pyrrolidine-2,5-dione

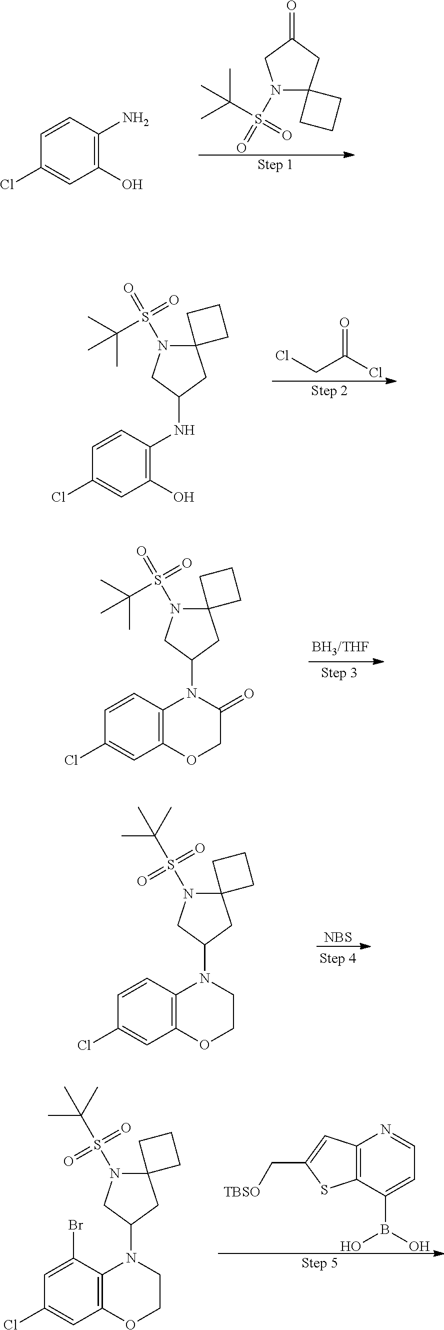

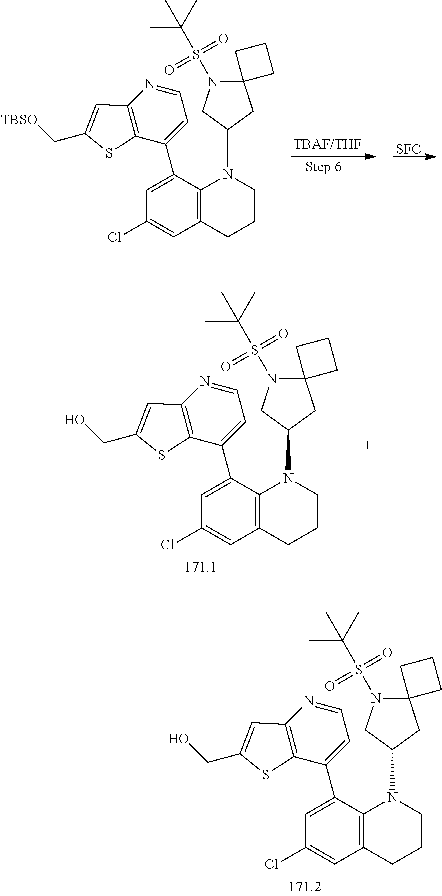 [SMILES: CC(C)(C)[Si](C)(C)OCc1cc2nccc(-c3cc(Cl)cc4c3N(C3CN(S(=O)(=O)C(C)(C)C)C5(CCC5)C3)CCC4)c2s1 ; "SFC" ; CC(C)(C)S(=O)(=O)N1C[C@H](N2CCCc3cc(Cl)cc(-c4ccnc5cc(CO)sc45)c32)CC12CCC2 ; CC(C)(C)S(=O)(=O)N1C[C@@H](N2CCCc3cc(Cl)cc(-c4ccnc5cc(CO)sc45)c32)CC12CCC2]

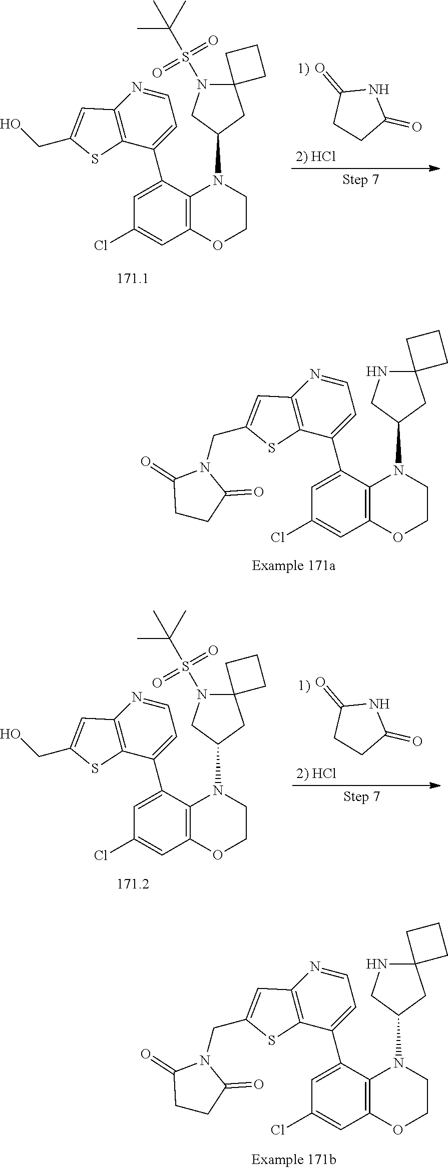 [SMILES: CC(C)(C)S(=O)(=O)N1C[C@H](N2CCOc3cc(Cl)cc(-c4ccnc5cc(CO)sc45)c32)CC12CCC2 ; O=C1CCC(=O)N1 ; O=C1CCC(=O)N1Cc1cc2nccc(-c3cc(Cl)cc4c3N([C@H]3CNC5(CCC5)C3)CCO4)c2s1 ; CC(C)(C)S(=O)(=O)N1C[C@@H](N2CCOc3cc(Cl)cc(-c4ccnc5cc(CO)sc45)c32)CC12CCC2 ; O=C1CCC(=O)N1 ; O=C1CCC(=O)N1Cc1cc2nccc(-c3cc(Cl)cc4c3N([C@@H]3CNC5(CCC5)C3)CCO4)c2s1]

### Step 1: 2-((5-(tert-butylsulfonyl)-5-azaspiro[3.4]octan-7-yl)amino)-5-chlorophenol

To a solution of 2-amino-5-chloro-phenol (2.6 g, 18.34 mmol) in acetic acid (40 mL) was added 5-(tert-butylsulfonyl)-5-azaspiro[3.4]octan-7-one (3.0 g, 12.23 mmol) and sodium triacetoxyborohydride (5.2 g, 24.46 mmol) at 25° C. After stirred at 25° C. for 2 h, the reaction mixture was concentrated under reduced pressure. Then the concentrate was quenched by addition of saturated aqueous sodium bicarbonate (20 mL) and extracted with ethyl acetate (20 mL×3). The combined organic layers were washed with brine (25 mL), dried over anhydrous sodium sulfate and concentrated under reduced pressure. The residue was purified by column chromatography to give the title compound. LCMS R_{T}=0.585 min, m/z=373.1 [M+H]⁺.

### Step 2: 4-(5-(tert-butylsulfonyl)-5-azaspiro[3.4]octan-7-yl)-7-chloro-2H-benzo[b][1,4]oxazin-3(4H)-one

To a solution of 2-((5-(tert-butylsulfonyl)-5-azaspiro[3.4]octan-7-yl)amino)-5-chlorophenol (1.3 g, 3.35 mmol) in acetonitrile (20 mL) was added potassium hydroxide (564 mg, 10.06 mmol) and 2-chloroacetyl chloride (379 mg, 3.35 mmol). After stirred at 80° C. for 0.5 h, the reaction mixture was concentrated under reduced pressure. Then the residue was quenched by addition of brine (20 mL) and extracted with ethyl acetate (15 mL×3). The combined organic layers were dried over anhydrous sodium sulfate and concentrated under reduced pressure. The residue was purified by column chromatography to give the title compound. LCMS R_{T}=0.620 min, m/z=413.1 [M+H]⁺.

### Step 3: 4-(5-(tert-butylsulfonyl)-5-azaspiro[3.4]octan-7-yl)-7-chloro-3,4-dihydro-2H-benzo[b][1,4]oxazine

To a solution of 4-(5-(tert-butylsulfonyl)-5-azaspiro[3.4]octan-7-yl)-7-chloro-2H-benzo[b][1,4]oxazin-3(4H)-one (1.0 g, 2.42 mmol) in THF (10 mL) was added borane-THF (1 M, 7.27 mL). After stirred at 60° C. for 12 h, the reaction mixture was diluted with methyl alcohol (20 mL) and concentrated under reduced pressure to give the crude title compound which was used in next step without further purification. LCMS R_{T}=0.623 min, m/z=399.2 [M+H]⁺.

### Step 4: 5-bromo-4-(5-(tert-butylsulfonyl)-5-azaspiro[3.4]octan-7-yl)-7-chloro-3,4-dihydro-2H-benzo[b][1,4]oxazine

To a solution of 4-(5-(tert-butylsulfonyl)-5-azaspiro[3.4]octan-7-yl)-7-chloro-3,4-dihydro-2H-benzo[b][1,4]oxazine (360 mg, 0.90 mmol) in dichloromethane (4 mL) was added N-bromosuccinimide (161 mg, 0.90 mmol). After stirred at 0° C. for 0.5 h, the reaction mixture was concentrated under reduced pressure. Then the residue was diluted with water (10 mL), extracted with ethyl acetate (5 mL×3). The combined organic layers were washed with brine (15 mL), dried over anhydrous sodium sulfate and concentrated under reduced pressure to give the crude title compound which was used in next step without further purification. LCMS R_{T}=0.675 min, m/z=479.0 [M+2+H]⁺.

### Step 5: 5-(2-(((tert-butyldimethylsilyl)oxy)methyl)thieno[3,2-b]pyridin-7-yl)-4-(5-(tert-butylsulfonyl)-5-azaspiro[3.4]octan-7-yl)-7-chloro-3,4-dihydro-2H-benzo[b][1,4]oxazine

The mixture of 5-bromo-4-(5-(tert-butylsulfonyl)-5-azaspiro[3.4]octan-7-yl)-7-chloro-3,4-dihydro-2H-benzo[b][1,4]oxazine (430 mg, 0.90 mmol), (2-(((tert-butyldimethylsilyl)oxy)methyl)thieno[3,2-b]pyridin-7-yl)boronic acid (436 mg, 1.35 mmol), dichloro[1,1′-bis(diphenylphosphino)ferrocene]palladium(II) (132 mg, 0.18 mmol) and potassium carbonate (311 mg, 2.25 mmol) in water (2 mL) and dioxane (20 mL) was stirred at 120° C. for 6 h. Then the reaction mixture was diluted with water (20 mL), extracted with ethyl acetate (15 mL×2). The combined organic layers were washed with brine (25 mL), dried over anhydrous sodium sulfate and concentrated under reduced pressure. The residue was purified by column chromatography to give the title compound. LCMS R_{T}=0.650 min, m/z=676.4 [M+H]⁺

### Step 6: (R)-(7-(4-(5-(tert-butylsulfonyl)-5-azaspiro[3.4]octan-7-yl)-7-chloro-3,4-dihydro-2H-benzo[b][1,4]oxazin-5-yl)thieno[3,2-b]pyridin-2-yl)methanol and (S)-(7-(4-(5-(tert-butylsulfonyl)-5-azaspiro[3.4]octan-7-yl)-7-chloro-3,4-dihydro-2H-benzo[b][1,4]oxazin-5-yl)thieno[3,2-b]pyridin-2-yl)methanol

To a solution of 5-(2-(((tert-butyldimethylsilyl)oxy)methyl)thieno[3,2-b]pyridin-7-yl)-4-(5-(tert-butylsulfonyl)-5-azaspiro[3.4]octan-7-yl)-7-chloro-3,4-dihydro-2H-benzo[b][1,4]oxazine (400 mg, 0.60 mmol) in THF (5 mL) was added tetrabutylammonium fluoride (0.8 mL, 1.0 M in THF). After stirred for 30 min at 25° C., the reaction mixture was concentrated under reduced pressure. The residue was purified by column chromatography. The racemate was separated by SFC to give first eluting fraction (171.1, Rt=4.151 min; LCMS R_{T}=0.551 min, m/z=562.2 [M+H]+) and second eluting fraction (171.2, Rt=3.722 min; LCMS R₁=0.551 min, m/z=562.2 [M+H]⁺.)

### Step 7

Example 171a was prepared from 171.1 and succinimide, following the procedure described in the synthesis of Example 108. The product was purified by preparative HPLC. ¹H NMR (400 MHz, CD₃OD) δ=8.69-8.65 (m, 1H), 7.50 (s, 1H), 6.96-6.92 (m, 1H), 6.87 (d, J=2.4 Hz, 1H), 4.96 (s, 2H), 4.18-4.09 (m, 2H), 3.27-3.22 (m, 2H), 2.74 (s, 5H), 2.45-2.30 (m, 1H), 1.97-1.07 (m, 10H). LCMS R_{T}=0.450 min, m/z=523.2 [M+H]+

Example 171b was prepared from 171.2 and succinimide, following the procedure described in the synthesis of Example 108. The product was purified by preparative HPLC. ¹H NMR (400 MHz, CD₃OD) δ=8.69-8.65 (m, 1H), 7.52-7.48 (m, 1H), 7.43-7.39 (m, 1H), 6.96-6.92 (m, 1H), 6.89-6.85 (m, 1H), 4.98-4.94 (m, 1H), 4.19-4.09 (m, 2H), 3.27-3.22 (m, 2H), 2.74 (s, 4H), 2.48-2.20 (m, 2H), 1.94-1.84 (m, 2H), 1.59-0.80 (m, 8H). LCMS R_{T}=0.543 min, m/z=523.2 [M+H]⁺. The absolute configuration was tentatively assigned based on the relative potency of the pair diastereomers in biology assays.

### Example 172: 3-((7-(7-chloro-4-((S)-5-azaspiro[3.4]octan-7-yl)-3,4-dihydro-2H-benzo[b][1,4]oxazin-5-yl)thieno[3,2-b]pyridin-2-yl)methyl)-6,6-dimethyl-3-azabicyclo[3.1.0]hexane-2,4-dione, hydrochloride acid salt

Example 172 was prepared from 171.2 and 6,6-dimethyl-3-azabicyclo[3.1.0]hexane-2,4-dione, following the procedure described in the synthesis of Example 108. The product was purified by preparative HPLC. ¹H NMR (400 MHz, CD₃OD) δ=8.91 (d, J=6.0, 1H), 7.84 (d, J=4.8, 1H), 7.7 (m, 1H), 7.12-7.11 (m, J=2.4, 1H), 7.04-7.03 (m, J=2.4, 1H), 4.87 (m, 2H), 4.35-4.13 (m, 2H), 3.55-3.39 (m, 2H), 2.89-2.69 (m, 1H), 2.62-2.51 (m, 2H), 2.42-1.45 (m, 10H), 1.31-1.25 (m, 3H), 1.21-1.15 (m, 3H). LCMS R_{T}=1.497 min, m/z=563.4 [M+H]⁺.

### Example 173a and 173b: (S)-1-((7-(7-chloro-3-oxo-4-(5-azaspiro[3.4]octan-7-yl)-3,4-dihydro-2H-benzo[b][1,4]oxazin-5-yl)thieno[3,2-b]pyridin-2-yl)methyl)pyrrolidine-2,5-dione, formic acid salt and (R)-1-((7-(7-chloro-3-oxo-4-(5-azaspiro[3.4]octan-7-yl)-3,4-dihydro-2H-benzo[b][1,4]oxazin-5-yl)thieno[3,2-b]pyridin-2-yl)methyl)pyrrolidine-2,5-dione, formic acid salt

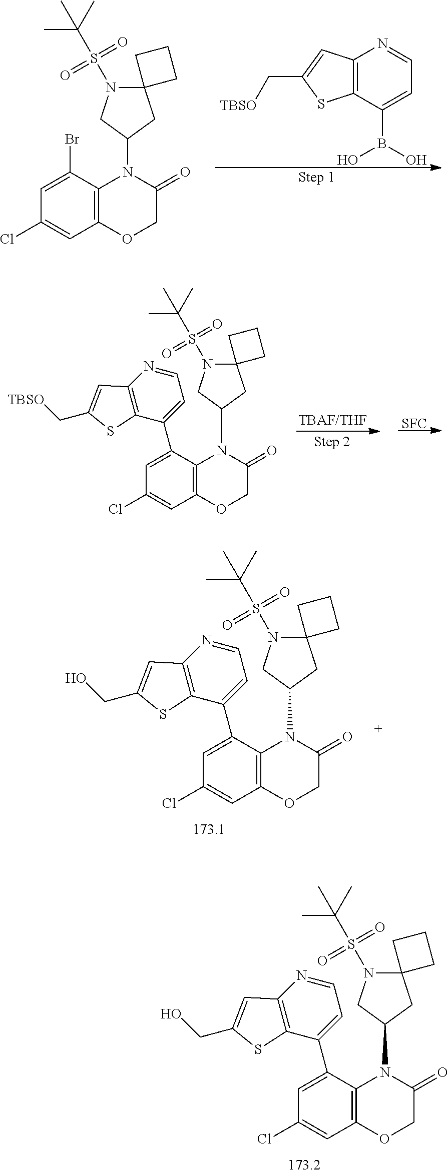

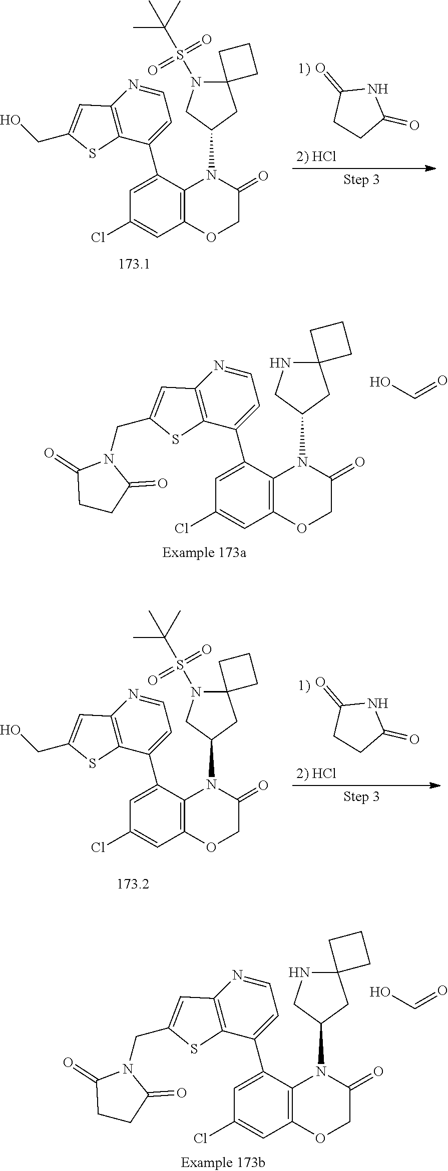

### Step 1: 5-(2-(((tert-butyldimethylsilyl)oxy)methyl)thieno[3,2-b]pyridin-7-yl)-4-(5-(tert-butylsulfonyl)-5-azaspiro[3.4]octan-7-yl)-7-chloro-2H-benzo[b][1,4]oxazin-3(4H)-one

The mixture of 5-bromo-4-(5-tert-butylsulfonyl-5-azaspiro[3.4]octan-7-yl)-7-chloro-1,4-benzoxazin-3-one (400 mg, 0.81 mmol), (2-(((tert-butyldimethylsilyl)oxy)methyl)thieno[3,2-b]pyridin-7-yl)boronic acid (316 mg, 0.98 mmol), dichloro[1,1′-bis(diphenylphosphino)ferrocene]palladium(II) (119 mg, 0.18 mmol) and potassium carbonate (282 mg, 2.03 mmol) in water (2 mL) and dioxane (5 mL) was stirred at 120° C. for 6 h. Then the reaction mixture was diluted with water (20 mL), extracted with ethyl acetate (15 mL×2). The combined organic layers were washed with brine (25 mL), dried over anhydrous sodium sulfate and concentrated under reduced pressure. The residue was purified by column chromatography to afford the title compound. LCMS R_{T}=0.681 min, m/z=690.4 [M+H]⁺

### Step 2: (S)-4-(5-(tert-butylsulfonyl)-5-azaspiro[3.4]octan-7-yl)-7-chloro-5-(2-(hydroxymethyl)thieno[3,2-b]pyridin-7-yl)-2H-benzo[b][1,4]oxazin-3(4H)-one and (R)-4-(5-(tert-butylsulfonyl)-5-azaspiro[3.4]octan-7-yl)-7-chloro-5-(2-(hydroxymethyl)thieno[3,2-b]pyridin-7-yl)-2H-benzo[b][1,4]oxazin-3(4H)-one

To a solution of 5-(2-(((tert-butyldimethylsilyl)oxy)methyl)thieno[3,2-b]pyridin-7-yl)-4-(5-(tert-butylsulfonyl)-5-azaspiro[3.4]octan-7-yl)-7-chloro-2H-benzo[b][1,4]oxazin-3(4H)-one (680 mg, 0.98 mmol) in THF (5 mL) was added tetrabutylammonium fluoride (1.3 mL, 1.0 M in THF). After stirred for 30 min at 25° C., the reaction mixture was concentrated under reduced pressure. The residue was purified by column chromatography. The racemate (270 mg, 0.47 mmol) was separated by SFC to afford first eluting fraction (173.1, Rt=1.683 min; LCMS R_{T}=0.558 min, m/z=576.1 [M+H]+) and second eluting fraction (173.2, R_{T}=2.191 min; LCMS Rt=0.558 min, m/z=576.1 [M+H]+).

### Step 3

Example 173a was prepared from 173.1 and succinimide, following the procedure described in the synthesis of Example 108. The product was purified by preparative HPLC. ¹H NMR (400 MHz, CD₃OD) δ=8.83 (d, J=4.4 Hz, 1H), 7.64-7.57 (m, 2H), 7.64-7.57 (m, 1H), 7.28 (d, J=2.0 Hz, 1H), 4.98 (d, J=4.4 Hz, 2H), 4.78-4.72 (m, 1H), 4.62 (d, J=11.2 Hz, 1H), 3.77 (d, J=4.0 Hz, 1H), 2.81-2.60 (m, 5H), 2.38-1.43 (m, 8H), 2.38-1.43 (m, 1H). LCMS R_{T}=1.327 min, m/z=537.3 [M+H]⁺.

Example 173b was prepared from 173.2 and succinimide, following the procedure described in the synthesis of Example 108. The product was purified by preparative HPLC. ¹H NMR (400 MHz, CD₃OD) δ=8.79 (d, J=4.8 Hz, 1H), 7.65-7.49 (m, 2H), 7.31 (s, 1H), 7.25 (d, J=2.4 Hz, 1H), 4.94 (d, J=4.8 Hz, 2H), 4.73-4.68 (m, 1H), 4.59-4.53 (m, 1H), 3.72-3.71 (m, 1H), 2.72 (d, J=4.4 Hz, 5H), 2.34-1.39 (m, 8H), 1.26-0.41 (m, 1H). LCMS R_{T}=1.458 min, m/z=537.0 [M+H]⁺. The absolute configuration was tentatively assigned based on the relative potency of the pair diastereomers in biology assays.

### Example 174: (S)-7-chloro-5-(2-((4-cyclopropyl-6-oxopyridazin-1(6H)-yl)methyl)thieno[3,2-b]pyridin-7-yl)-4-(5-azaspiro[3.4]octan-7-yl)-2H-benzo[b][1,4]oxazin-3(4H)-one, formic acid salt

Example 174 was prepared from 173.2 and 4-cyclopropyl-1H-pyridazin-6-one, following the procedure described in the synthesis of Example 108. The product was purified by preparative HPLC. ¹H NMR (400 MHz, CD₃OD) δ=8.82 (d, J=4.8 Hz, 1H), 7.84-7.74 (m, 1H), 7.72-7.64 (m, 1H), 7.59 (dd, J=8.0 Hz, 4.8 Hz, 1H), 7.34 (s, 1H), 7.27 (d, J=2.4 Hz, 1H), 6.58 (s, 1H), 5.71-5.54 (m, 2H), 4.77-4.52 (m, 2H), 3.80-3.60 (m, 1H), 2.67-2.41 (m, 1H), 2.32-2.02 (m, 2H), 1.98-1.64 (m, 5H), 1.60-1.14 (m, 4H), 0.95 (d, J=4.4 Hz, 2H), 0.44 (dd, J=12.8 Hz, 8.8 Hz, 1H). LCMS R_{T}=1.692 min, m/z=574.1 [M+H]⁺.

### Example 175a and 175b: (R)-1-((7-(4-(5-azaspiro[3.4]octan-7-yl)-7-(trifluoromethyl)-3,4-dihydro-2H-benzo[b][1,4]oxazin-5-yl)thieno[3,2-b]pyridin-2-yl)methyl)pyrrolidine-2,5-dione, hydrochloride and (S)-1-((7-(4-(5-azaspiro[3.4]octan-7-yl)-7-(trifluoromethyl)-3,4-dihydro-2H-benzo[b][1,4]oxazin-5-yl)thieno[3,2-b]pyridin-2-yl)methyl)pyrrolidine-2,5-dione, hydrochloride

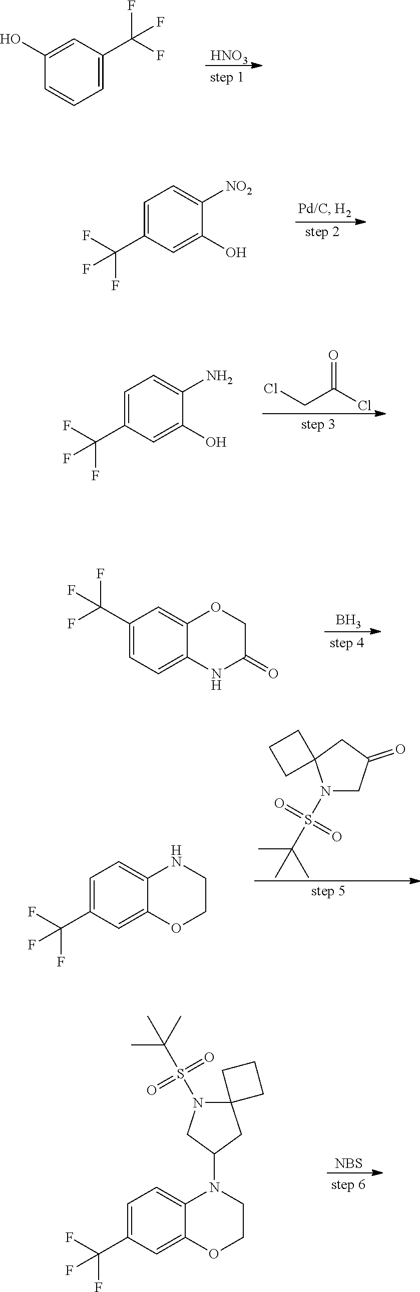

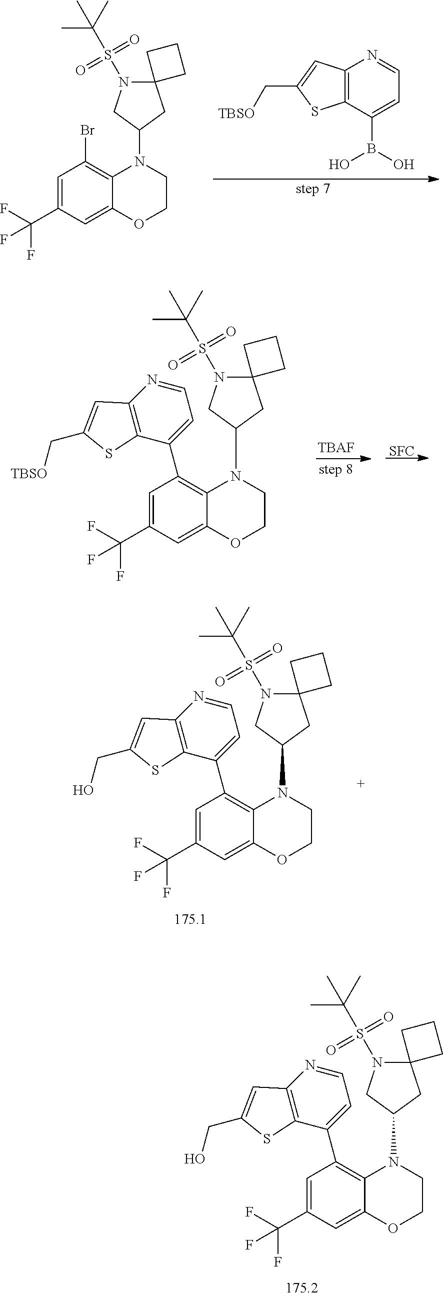

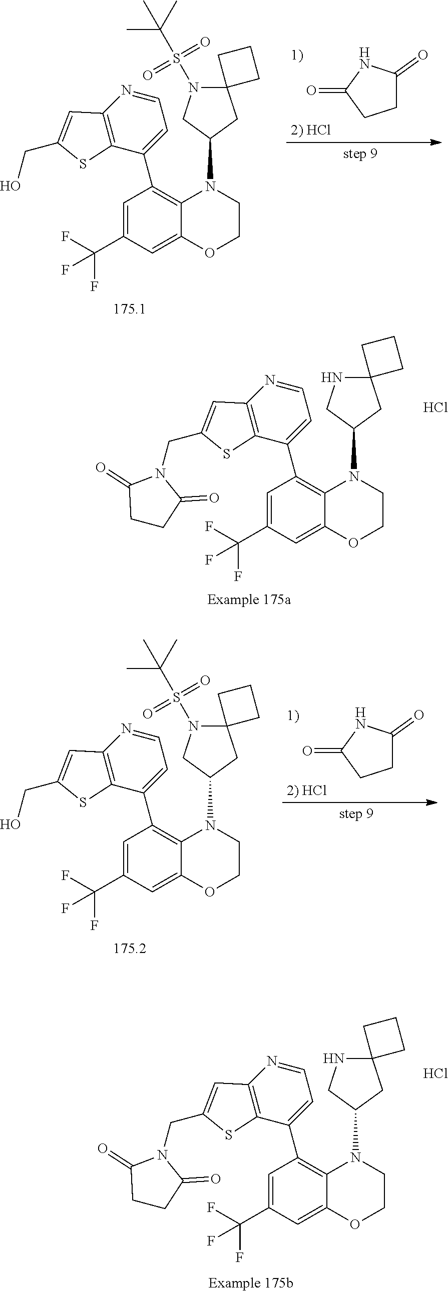

### Step 1: 2-nitro-5-(trifluoromethyl)phenol

To a solution of 3-(trifluoromethyl)phenol (9.0 g, 55.52 mmol) in acetic acid (18 mL) was added nitric acid (5.9 g, 60.65 mmol) in acetic acid (100 mL) dropwise over 15 min at 40° C. After stirred at 40° C. for 1 h, the mixture was quenched by addition of ice water (100 mL), then extracted with ethyl acetate (200 mL). The combined organic layers were dried over anhydrous sodium sulfate and concentrated under reduced pressure. The residue was purified by column chromatography to afford the title compound. ¹HNMR (400 MHz, CDCl₃) δ=10.47-10.36 (m, 1H), 8.10-8.01 (m, 1H), 7.31-7.23 (m, 1H), 7.12-7.00 (m, 1H).

### Step 2: 2-amino-5-(trifluoromethyl)phenol

To a solution of 2-nitro-5-(trifluoromethyl)phenol (5.0 g, 24.14 mmol) in ethyl alcohol (50 mL) was added 10% palladium on carbon (1.0 g). After stirred under hydrogen atmosphere (15 Psi) at 80° C. for 2 h, the reaction mixture was filtered and the filtrate was concentrated under reduced pressure to give a residue. The residue was purified by column chromatography to afford the title compound. LCMS R_{T}=0.966 min, m/z=178.2 [M+H]⁺.

### Step 3: 7-(trifluoromethyl)-2H-benzo[b][1,4]oxazin-3(4H)-one

To a solution of 2-amino-5-(trifluoromethyl)phenol (1.9 g, 10.73 mmol) and potassium carbonate (4.5 g, 32.18 mmol) in acetonitrile (12 mL) was added 2-chloroacetyl chloride (1.3 g, 11.80 mmol) under nitrogen atmosphere. After stirred at 85° C. for 2 h, the reaction mixture was concentrated under reduced pressure to remove acetonitrile. The residue was diluted with water (30 mL) and extracted with ethyl acetate (60 mL). The combined organic layers were dried over anhydrous sodium sulfate, filtered and the filtrate was concentrated under reduced pressure to give a residue. The residue was purified by column chromatography to afford the title compound. LCMS R_{T}=1.298 m/z=218.0 [M+H]⁺.

### Step 4: 7-(trifluoromethyl)-3,4-dihydro-2H-benzo[b][1,4]oxazine

To a solution of 7-(trifluoromethyl)-2H-benzo[b][1,4]oxazin-3(4H)-one (1.6 g, 7.37 mmol) in THF (10 mL) was added borane THF (1 M, 29.47 mL). After stirred at 70° C. for 2 h, the reaction mixture was quenched by addition of methanol (15 mL) and water (40 mL) and extracted with ethyl acetate (120 mL×3). The combined organic layers were dried over anhydrous sodium sulfate, filtered and the filtrate was concentrated under reduced pressure to give a residue. The residue was purified by column chromatography to afford the title compound. LCMS R_{T}=1.195 min, m/z=204.2 [M+H]⁺.

### Step 5: 4-(5-(tert-butylsulfonyl)-5-azaspiro[3.4]octan-7-yl)-7-(trifluoromethyl)-3,4-dihydro-2H-benzo[b][1,4]oxazine

To a solution of 7-(trifluoromethyl)-3,4-dihydro-2H-benzo[b][1,4]oxazine (2.2 g, 10.83 mmol) and 5-(tert-butylsulfonyl)-5-azaspiro[3.4]octan-7-one (3.2 g, 12.99 mmol) in methanol (20 mL) was added indium chloride (718 mg, 3.25 mmol) and triethyl silicane (7.5 g, 32.49 mmol) at 25° C. After stirred at 25° C. for 16 h, the reaction mixture was concentrated under reduced pressure. The residue was purified by preparative HPLC to afford the title compound. LCMS R_{T}=0.643 min, m/z=433.3 [M+H]⁺.

### Step 6: 5-bromo-4-(5-(tert-butylsulfonyl)-5-azaspiro[3.4]octan-7-yl)-7-(trifluoromethyl)-3,4-dihydro-2H-benzo[b][1,4]oxazine

To a solution of 4-(5-(tert-butylsulfonyl)-5-azaspiro[3.4]octan-7-yl)-7-(trifluoromethyl)-3,4-dihydro-2H-benzo[b][1,4]oxazine (280 mg, 0.65 mmol) in DMF (5 mL) was added N-bromosuccinimide (127 mg, 0.71 mmol) at 0° C. After stirred at 0° C. for 1 h, the reaction mixture was diluted with water (20 mL) and extracted with ethyl acetate (40 mL×3). The combined organic layers were dried over anhydrous sodium sulfate, filtered and the filtrate was concentrated under reduced pressure to give a residue. The residue was purified by column chromatography to afford the title compound. LCMS R_{T}=0.672 min, m/z=511.1 [M+H]⁺.

### Step 7: 5-(2-(((tert-butyldimethylsilyl)oxy)methyl)thieno[3,2-b]pyridin-7-yl)-4-(5-(tert-butylsulfonyl)-5-azaspiro[3.4]octan-7-yl)-7-(trifluoromethyl)-3,4-dihydro-2H-benzo[b][1,4]oxazine

To a solution of 5-bromo-4-(5-(tert-butylsulfonyl)-5-azaspiro[3.4]octan-7-yl)-7-(trifluoromethyl)-3,4-dihydro-2H-benzo[b][1,4]oxazine (310 mg, 0.61 mmol) and (2-(((tert-butyldimethylsilyl)oxy)methyl)thieno[3,2-b]pyridin-7-yl)boronic acid (255 mg, 0.79 mmol) in dioxane (8 mL) and water (0.8 mL) was added potassium carbonate (251 mg, 1.82 mmol) and [1,1′-bis(diphenylphosphino)ferrocene]dichloropalladium(II) (133 mg, 0.18 mmol) at 25° C. After stirred at 100° C. for 2 h under nitrogen atmosphere, the reaction mixture was concentrated under reduced pressure to remove the solvent. The residue was purified by column chromatography to afford the title compound. LCMS R_{T}=0.653 min, m/z=710.2 [M+H]⁺.

### Step 8: (R)-(7-(4-(5-(tert-butylsulfonyl)-5-azaspiro[3.4]octan-7-yl)-7-(trifluoromethyl)-3,4-dihydro-2H-benzo[b][1,4]oxazin-5-yl)thieno[3,2-b]pyridin-2-yl)methanol and (S)-(7-(4-(5-(tert-butylsulfonyl)-5-azaspiro[3.4]octan-7-yl)-7-(trifluoromethyl)-3,4-dihydro-2H-benzo[b][1,4]oxazin-5-yl)thieno[3,2-b]pyridin-2-yl)methanol

To a solution of 5-(2-(((tert-butyldimethylsilyl)oxy)methyl)thieno[3,2-b]pyridin-7-yl)-4-(5-(tert-butylsulfonyl)-5-azaspiro[3.4]octan-7-yl)-7-(trifluoromethyl)-3,4-dihydro-2H-benzo[b][1,4]oxazine (672 mg, 0.67 mmol) in THF (3 mL) was added tetrabutyl ammonium fluoride (1 M, 0.66 mL). After stirred at 25° C. for 0.5 h. The reaction mixture was concentrated under reduced pressure to remove the solvent. The residue was purified by column chromatography. The racemate was separated by SFC to give first eluting fraction (175.1, Rt=0.547 min; LCMS RT=0.547 min, m/z=596.2 [M+H]+) and second eluting fraction (175.2, Rt=0.528 min; LCMS RT=0.528 min, m/z=596.3 [M+H]+).

### Step 9

Example 175a was prepared from 175.1 and succinimide, following the procedure described in the synthesis of Example 108. The product was purified by preparative HPLC. ¹H NMR (400 MHz, CD₃OD) δ=9.05-8.96 (m, 1H), 8.10-7.74 (m, 2H), 7.39-7.29 (m, 2H), 5.14-5.04 (m, 2H), 4.56-4.12 (m, 2H), 3.62-3.35 (m, 3H), 2.82-2.78 (m, 4H), 2.70-2.07 (m, 4H), 2.04-0.90 (m, 6H). LCMS R_{T}=1.348 min, m/z=557.3 [M+H]⁺.

Example 175b was prepared from 175.2 and succinimide, following the procedure described in the synthesis of Example 108. The product was purified by preparative HPLC. ¹H NMR (400 MHz, CD₃OD) δ=9.03-8.95 (m, 1H), 8.04-7.72 (m, 2H), 7.40-7.28 (m, 2H), 5.11-5.03 (m, 2H), 4.45-4.14 (m, 2H), 3.62-3.36 (m, 3H), 2.81-2.78 (m, 4H), 2.41-1.83 (m, 6H), 1.77-1.42 (m, 4H). LCMS R_{T}=1.355 min, m/z=557.3 [M+H]⁺.

The absolute configuration was tentatively assigned based on the relative potency of the pair diastereomers in biology assays.

### Example 176a and 179b: (R)-2-((7-(4-(5-azaspiro[3.4]octan-7-yl)-7-(trifluoromethyl)-3,4-dihydro-2H-benzo[b][1,4]oxazin-5-yl)thieno[3,2-b]pyridin-2-yl)methyl)-4-methylpyridazin-3(2H)-one and (S)-2-((7-(4-(5-azaspiro[3.4]octan-7-yl)-7-(trifluoromethyl)-3,4-dihydro-2H-benzo[b][1,4]oxazin-5-yl)thieno[3,2-b]pyridin-2-yl)methyl)-4-methylpyridazin-3(2H)-one

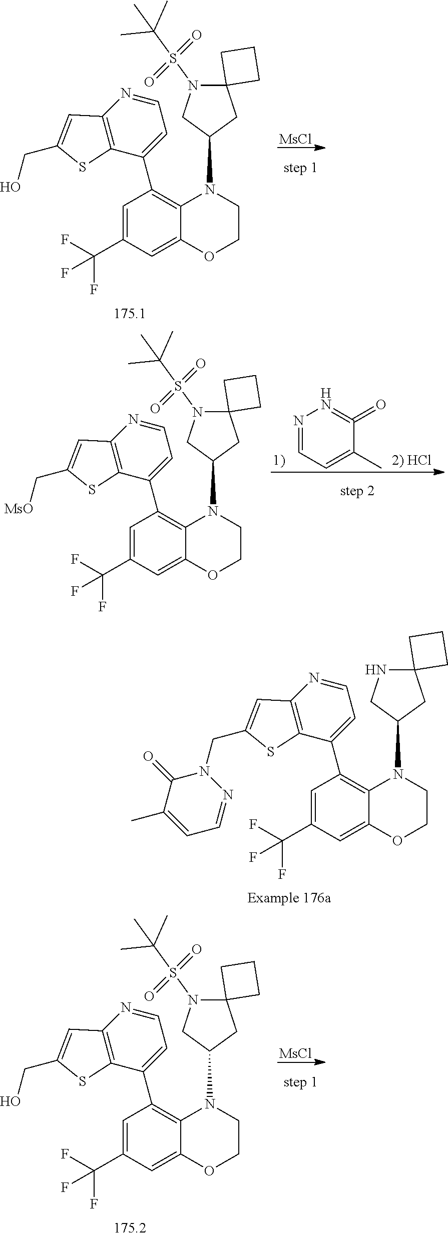

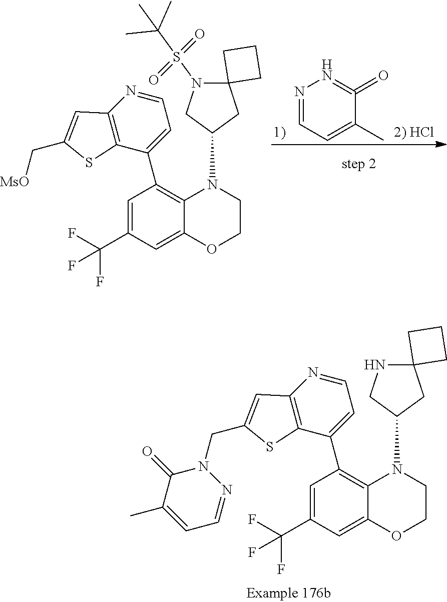

### Step 1: (R)-(7-(4-(5-(tert-butylsulfonyl)-5-azaspiro[3.4]octan-7-yl)-7-(trifluoromethyl)-3,4-dihydro-2H-benzo[b][1,4]oxazin-5-yl)thieno[3,2-b]pyridin-2-yl)methyl methanesulfonate

To a solution of 175.1 (50 mg, 0.08 mmol) in methylene chloride (3 mL) was added methane sulfonyl chloride (28 mg, 0.25 mmol) and triethylamine (25 mg, 0.25 mmol) at 20° C. After stirred at 20° C. for 1 h, the mixture was quenched by addition of saturated aqueous sodium bicarbonate (5 mL), extracted with methylene chloride (5 mL×4), dried over anhydrous sodium sulfate and concentrated under reduced pressure. The combined organic layers were dried over anhydrous sodium sulfate and concentrated under reduced pressure to afford the title compound which was used in next step without further purification. LCMS R_{T}=0.607 min, m/z=674.2 [M+H]⁺.

### Step 2

To a solution of (R)-(7-(4-(5-(tert-butylsulfonyl)-5-azaspiro[3.4]octan-7-yl)-7-(trifluoromethyl)-3,4-dihydro-2H-benzo[b][1,4]oxazin-5-yl)thieno[3,2-b]pyridin-2-yl)methyl methanesulfonate (50 mg, 0.07 mmol) and 4-methylpyridazin-3(2H)-one (16 mg, 0.14 mmol) in acetonitrile (1 mL) was added 1,8-diazabicyclo[5.4.0]undec-7-ene (33 mg, 0.22 mmol) at 20° C. After stirred at 20° C. for 1 h, the reaction mixture was concentrated under reduced pressure to remove the solvent. The residue was added hydrochloric acid (12 M, 3 mL). After stirred at 25° C. for 30 min, the reaction mixture was concentrated under reduced pressure. The residue was purified by preparative HPLC to afford Example 176a. ¹H NMR (400 MHz, CD₃OD) δ=0.96-2.21 (m, 10H) 2.25 (s, 3H) 2.34-3.04 (m, 3H) 3.49 (s, 1H) 4.17 (s, 1H) 5.62 (s, 2H) 6.80 (s, 1H) 7.14-7.25 (m, 2H) 7.40-7.70 (m, 2H) 7.88 (d, J=1.6 Hz, 1H) 8.75 (d, J=4.0 Hz, 1H). LCMS R_{T}=2.299 m, m/z=568.4 [M+H]⁺.

Example 176b was prepared from 175.2, following the procedure described in the synthesis of Example 179a. The final product was purified by preparative HPLC. ¹H NMR (400 MHz, CD₃OD) δ=8.75 (d, J=4.8 Hz, 1H), 7.87 (d, J=2.0 Hz, 1H), 7.41-7.69 (m, 2H), 7.12-7.28 (m, 2H), 6.79 (s, 1H), 5.61 (s, 2H), 4.16 (s, 1H), 3.49 (s, 1H), 2.31-3.07 (m, 3H), 2.25 (s, 3H), 0.89-2.20 (m, 9H). LCMS R_{T}=2.284 m, m/z=568.4 [M+H]⁺

### Example 177a and 177b: (R)-1-((7-(6-chloro-1-(2-fluoro-5-azaspiro[3.4]octan-7-yl)-1,2,3,4-tetrahydroquinolin-8-yl)thieno[3,2-b]pyridin-2-yl)methyl)pyrrolidine-2,5-dione, formic acid salt and (S)-1-((7-(6-chloro-1-(2-fluoro-5-azaspiro[3.4]octan-7-yl)-1,2,3,4-tetrahydroquinolin-8-yl)thieno[3,2-b]pyridin-2-yl)methyl)pyrrolidine-2,5-dione, formic acid salt

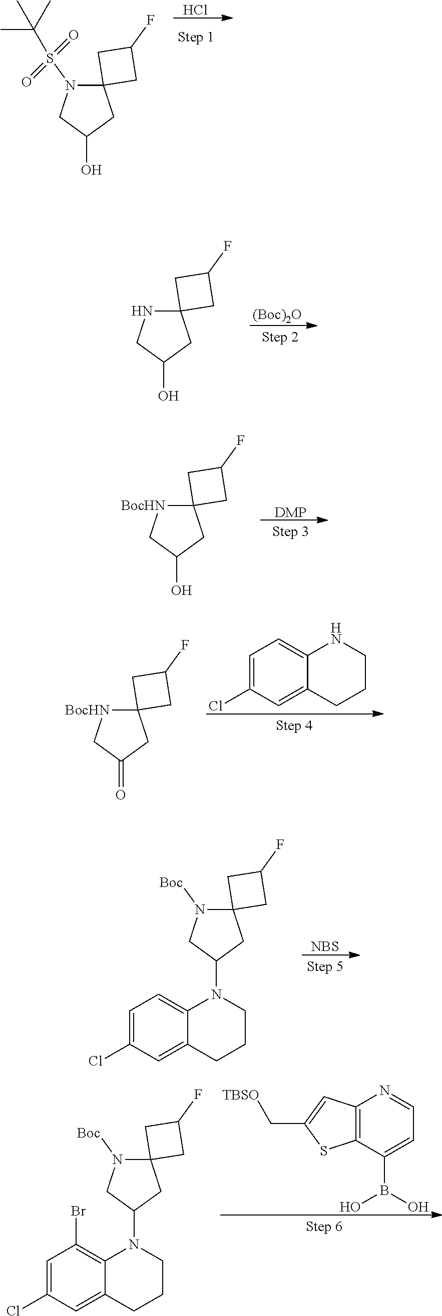

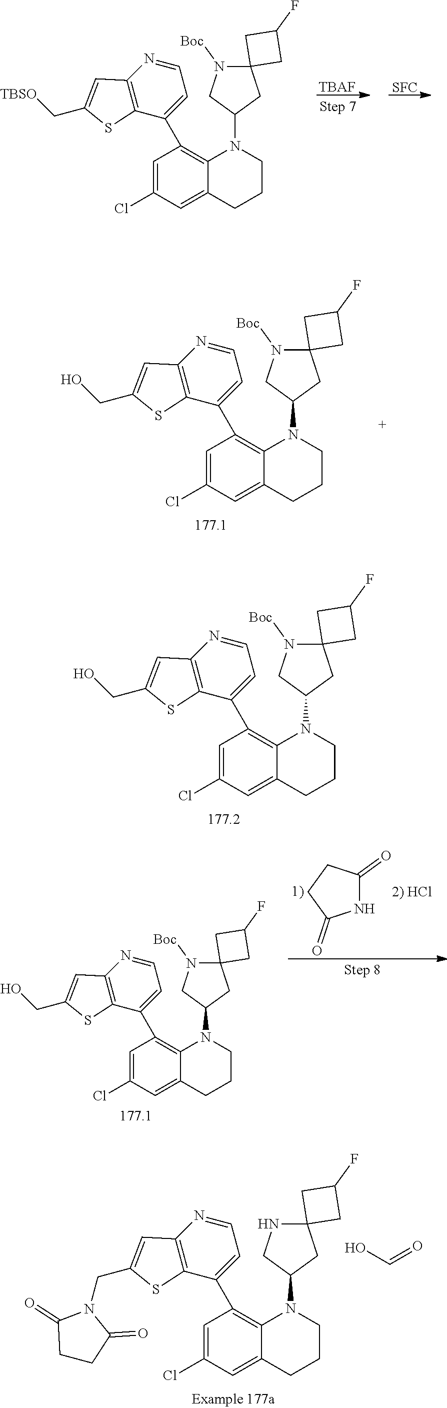

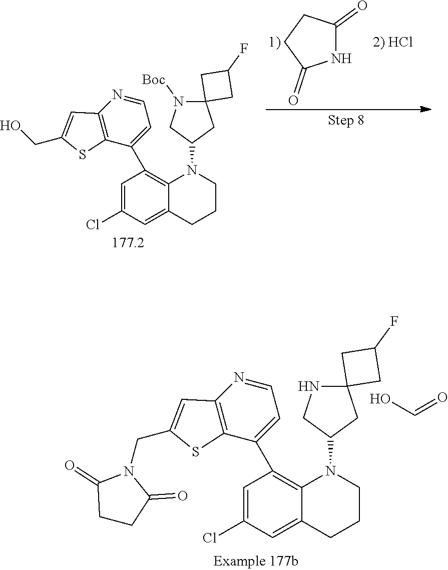

### Step 1: 2-fluoro-5-azaspiro[3.4]octan-7-ol

The solution of 5-(tert-butylsulfonyl)-2-fluoro-5-azaspiro[3.4]octan-7-ol (230 mg, 0.87 mmol) in hydrochloric acid (12 M, 5 mL) was stirred at 25° C. for 2 h. The reaction mixture was concentrated under reduced pressure to afford the title compound.

### Step 2: tert-butyl 2-fluoro-7-hydroxy-5-azaspiro[3.4]octane-5-carboxylate

To a solution of 2-fluoro-5-azaspiro[3.4]octan-7-ol (230 mg, 1.58 mmol) in THF (0.75 mL) and water (0.75 mL) was added trimethylamine (962 mg, 9.51 mmol) and di-tert-butyl dicarbonate (1.04 g, 4.75 mmol). After stirred at 25° C. for 12 h, the reaction mixture was concentrated under reduced pressure. The residue was purified by column chromatography to afford the title compound. LCMS R_{T}=0.505 min, m/z=190.2 [M+H−56]⁺.

### Step 3: tert-butyl 2-fluoro-7-oxo-5-azaspiro[3.4]octane-5-carboxylate

To a solution of tert-butyl 2-fluoro-7-hydroxy-5-azaspiro[3.4]octane-5-carboxylate (600 mg, 2.45 mmol) in dichloromethane (10 mL) was added Triacetoxy)-1,1-dihydro-1,2-benziodoxol-3(1H)-one (1.56 g, 3.67 mmol) at 20° C. After stirred at 20° C. for 1 h, the mixture was quenched by addition of saturated aqueous sodium hydrogen carbonate (30 mL) and extracted with dichloromethane (10 mL×4). The combined organic layers were washed with brine (60 mL), dried over sodium sulfate, filtered and the filtrate was concentrated under reduced pressure. The residue was purified by column chromatography to afford the title compound. ¹H NMR (400 MHz, CD₃OD) δ=5.44-5.12 (m, 1H), 3.85-3.62 (m, 2H), 3.61-3.27 (m, 2H), 2.89 (s, 2H), 2.45-2.16 (m, 2H), 1.43 (s, 9H).

### Step 4: tert-butyl 7-(6-chloro-3,4-dihydroquinolin-1(2H)-yl)-2-fluoro-5-azaspiro[3.4]octane-5-carboxylate

To a solution tert-butyl 2-fluoro-7-oxo-5-azaspiro[3.4]octane-5-carboxylate (260 mg, 1.07 mmol) in acetic acid (1 mL) and methanol (5 mL) was added 6-chloro-1,2,3,4-tetrahydroquinoline (215 mg, 1.28 mmol) and (2-methylpyridin-1-ium-1-yl)boranuide (227 mg, 2.14 mmol) at 20° C. After stirred at 20° C. for 1 h, the mixture was quenched by addition of 1N hydrogen chloride and extracted with dichloromethane (10 mL×4). The combined organic layers were washed with brine (10 mL), dried over anhydrous sodium sulfate, filtered and the filtrate was concentrated under reduced pressure. The residue was purified by column chromatography to afford the title compound. LCMS R_{T}=0.683 min, m/z=395.3 [M+H]⁺.

### Step 5: tert-butyl 7-(8-bromo-6-chloro-3,4-dihydroquinolin-1(2H)-yl)-2-fluoro-5-azaspiro[3.4]octane-5-carboxylate

To a solution of tert-butyl 7-(6-chloro-3,4-dihydroquinolin-1(2H)-yl)-2-fluoro-5-azaspiro[3.4]octane-5-carboxylate (240 mg, 0.61 mmol) in DMF (1 mL) was added 1-bromopyrrolidine-2,5-dione (87 mg, 0.49 mmol) at 20° C. After stirred at 20° C. for 1 h, the reaction mixture was diluted with water (20 mL) and extracted with ethyl acetate (10 mL×4). The combined organic layers were washed with brine (10 mL), dried over anhydrous sodium sulfate, filtered and the filtrate was concentrated under reduced pressure. The residue was purified by column chromatography to afford the title compound. ¹H NMR (400 MHz, CD₃OD) δ=7.40 (s, 1H), 7.02-6.95 (m, 1H), 5.45-5.06 (m, 1H), 4.10-4.00 (m, 1H), 3.81-3.52 (m, 2H), 3.44-2.98 (m, 4H), 2.83-2.65 (m, 2H), 2.53-2.11 (m, 4H), 1.81 (t, J=6.4 Hz, 2H), 1.54-1.44 (m, 9H). LCMS R_{T}=0.721 min, m/z=474.2 [M+H]⁺.

### Step 6: tert-butyl 7-(8-(2-(((tert-butyldimethylsilyl)oxy)methyl)thieno[3,2-b]pyridin-7-yl)-6-chloro-3,4-dihydroquinolin-1(2H)-yl)-2-fluoro-5-azaspiro[3.4]octane-5-carboxylate

To a solution of tert-butyl 7-(8-bromo-6-chloro-3,4-dihydroquinolin-1(2H)-yl)-2-fluoro-5-azaspiro[3.4]octane-5-carboxylate (250 mg, 0.53 mmol) in water (0.5 mL) and dioxane (5 mL) was added (2-(((tert-butyldimethylsilyl)oxy)methyl)thieno[3,2-b]pyridin-7-yl)boronic acid (205 mg, 0.63 mmol), dichloro[1,1′-bis(diphenylphosphino)ferrocene]palladium(II) (77 mg, 0.11 mmol) and dipotassium carbonate (73 mg, 0.53 mmol) at 20° C. under nitrogen atmosphere. After stirred at 100° C. for 2 h, the reaction mixture was filtered and the filtrate was concentrated under reduced pressure to remove the solvent. The residue was purified by column chromatography to afford the title compound. LCMS R_{T}=0.677 min, m/z=672.5 [M+H]⁺.

### Step 7: (R)-tert-butyl 7-(6-chloro-8-(2-(hydroxymethyl)thieno[3,2-b]pyridin-7-yl)-3,4-dihydroquinolin-1(2H)-yl)-2-fluoro-5-azaspiro[3.4]octane-5-carboxylate and (S)-tert-butyl 7-(6-chloro-8-(2-(hydroxymethyl)thieno[3,2-b]pyridin-7-yl)-3,4-dihydroquinolin-1(2H)-yl)-2-fluoro-5-azaspiro[3.4]octane-5-carboxylate

To a solution of tert-butyl 7-(8-(2-(((tert-butyldimethylsilyl)oxy)methyl)thieno[3,2-b]pyridin-7-yl)-6-chloro-3,4-dihydroquinolin-1(2H)-yl)-2-fluoro-5-azaspiro[3.4]octane-5-carboxylate (200 mg, 0.30 mmol) in THF (1 mL) was added tetrabutylammonium fluoride (1 M in THF, 1 mmol). After stirred at 20° C. for 0.5 h, the reaction mixture was concentrated under reduced pressure to remove solvent. The reaction mixture was diluted with water (20 mL) and extracted with ethyl acetate (10 mL×4). The combined organic layers were washed with brine (10 mL), dried over anhydrous sodium sulfate, filtered and the filtrate was concentrated under reduced pressure. The residue was purified by column chromatography. The racemate was separated by SFC to give first eluting fraction (177.1, Rt=1.474; LCMS R_{T}=0.543 min, m/z=558.3 [M+H]+) and second eluting fraction (177.2, Rt=1.617; LCMS R_{T}=0.547 min, m/z=558.3 [M+H]+).

### Step 8

Example 177a was prepared from 177.1 and succinimide, following the procedure described in the synthesis of Example 108. The product was purified by preparative HPLC. ¹H NMR (400 MHz, CD₃OD) δ=8.67 (d, J=4.8 Hz, 1H), 8.44 (s, 1H), 7.48 (s, 1H), 7.40 (d, J=4.8 Hz, 1H), 7.12 (dd, J=2.4, 12.8 Hz, 2H), 4.93 (s, 2H), 3.51 (d, J=8.0 Hz, 1H), 3.16 (d, J=13.6 Hz, 2H), 2.91-2.54 (m, 7H), 2.63-0.73 (m, 10H). LCMS: R_{T}=1.247 min, m/z=539.4 [M+H]⁺.

Example 177b was prepared from 177.2 and succinimide, following the procedure described in the synthesis of Example 108. The product was purified by preparative HPLC. ¹H NMR (400 MHz, CD₃OD) δ=8.67 (d, J=4.8 Hz, 1H), 8.44 (s, 1H), 7.48 (s, 1H), 7.40 (d, J=4.8 Hz, 1H), 7.11 (dd, J=2.4, 12.8 Hz, 2H), 4.93 (s, 2H), 3.51 (d, J=7.6 Hz, 1H), 3.16 (d, J=16.4 Hz, 2H), 2.91-2.67 (m, 7H), 2.65-1.19 (m, 10H). LCMS: R_{T}=1.233 min, m/z=539.2[M+H]⁺.

The absolute configuration was tentatively assigned based on the relative potency of the pair diastereomers in biology assays.

### Example 178a and 178b: 8-(2-((2,4-dioxo-3-azabicyclo[3.1.0]hexan-3-yl)methyl)thieno[3,2-b]pyridin-7-yl)-1-((R)-1-azaspiro[4.4]nonan-3-yl)-1,2,3,4-tetrahydroquinoline-6-carbonitrile, formic acid salt and 8-(2-((2,4-dioxo-3-azabicyclo[3.1.0]hexan-3-yl)methyl)thieno[3,2-b]pyridin-7-yl)-1-((S)-1-azaspiro[4.4]nonan-3-yl)-1,2,3,4-tetrahydroquinoline-6-carbonitrile

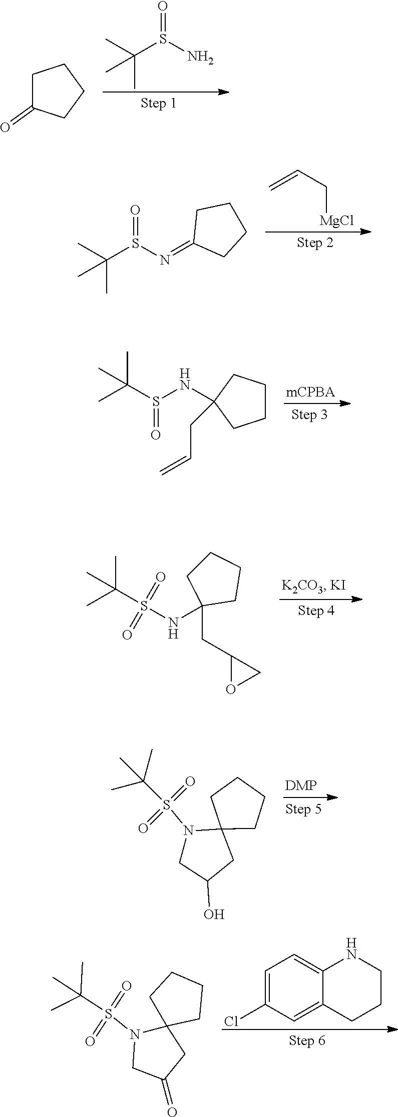

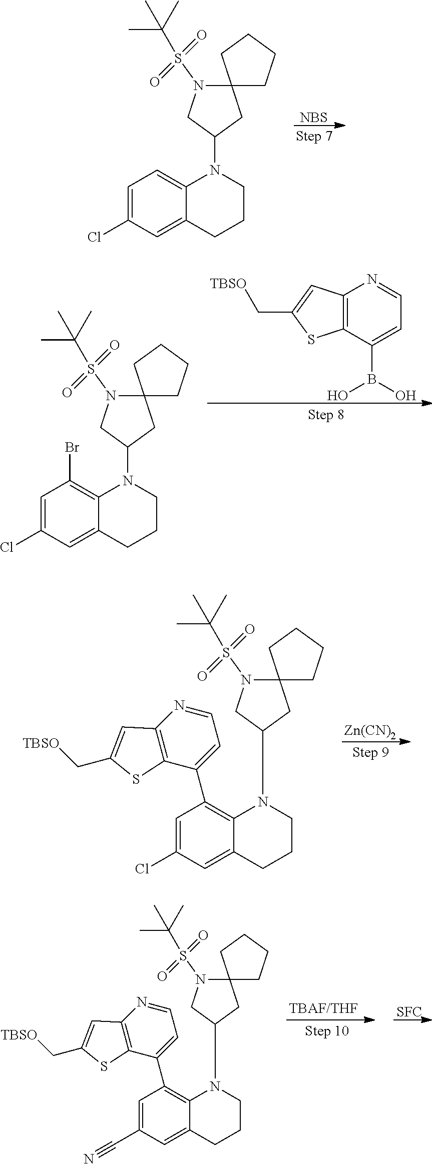

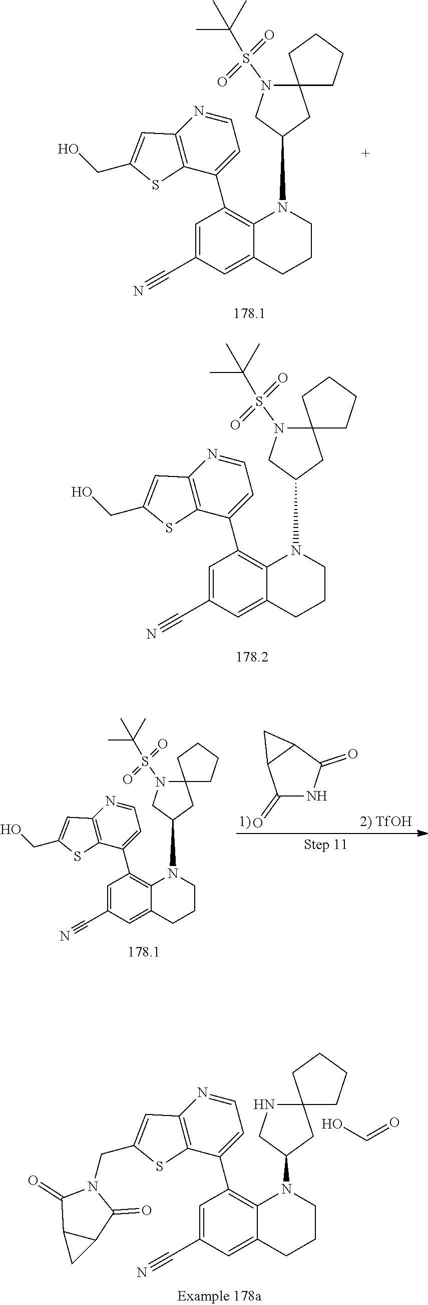

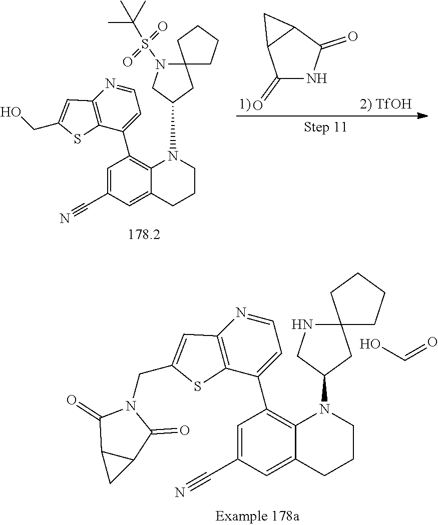

### Step 1: N-cyclopentylidene-2-methylpropane-2-sulfinamide

To a solution of cyclopentanone (30 g, 356.65 mmol) in THF (200 mL) was added 2-methylpropane-2-sulfinamide (43.23 g, 356.65 mmol) and titanium(iv) isopropoxide (152.05 g, 534.97 mmol) at 20° C. After stirred at 20° C. for 12 h, the mixture was quenched by addition of saturated aqueous sodium hydrogencarbonate (400 mL) and extracted with ethyl acetate (200 mL×4). The combined organic layers were washed with brine (100 mL), dried over anhydrous sodium sulfate, filtered and the filtrate was concentrated under reduced pressure. The residue was purified by column chromatography to afford the title compound. ¹H NMR (400 MHz, CD₃OD) δ=2.96-2.82 (m, 1H), 2.61-2.46 (m, 3H), 1.96-1.72 (m, 4H), 1.25-1.20 (m, 9H).

### Step 2: N-(1-allylcyclopentyl)-2-methylpropane-2-sulfinamide

To a solution of allylmagnesium chloride (400.42 mmol, 2 M in THF) in THF (200 mL) was added N-cyclopentylidene-2-methylpropane-2-sulfinamide (50 g, 266.95 mmol) at −78° C. After stirred at −78° C. for 3 h, the mixture was quenched by addition of saturated aqueous ammonium chloride (300 mL) and extracted with ethyl acetate (100 mL×4). The combined organic layers were washed with brine (100 mL), dried over anhydrous sodium sulfate, filtered and the filtrate was concentrated under reduced pressure. The residue was purified by column chromatography to afford the title compound. ¹H NMR (400 MHz, CD₃OD) δ=5.94-5.78 (m, 1H), 5.20-5.09 (m, 2H), 4.85-4.78 (m, 1H), 2.60-2.29 (m, 3H), 1.94-1.67 (m, 7H), 1.21-1.17 (m, 9H).

### Step 3: 2-methyl-N-(1-(oxiran-2-ylmethyl)cyclopentyl)propane-2-sulfonamide

To a solution of N-(1-allylcyclopentyl)-2-methylpropane-2-sulfinamide (40 g, 174.38 mmol) in dichloromethane (200 mL) was added 3-chloroperbenzoic acid (90.28 g, 523.15 mmol) at 20° C. After stirred at 20° C. for 12 h, the mixture was quenched by addition of saturated aqueous sodium thiosulfate/sodium hydrogencarbonate (100 mL) and extracted with dichloromethane (50 mL×4). The combined organic layers were washed with brine (100 mL), dried over anhydrous sodium sulfate, filtered and the filtrate was concentrated under reduced pressure. The residue was purified by column chromatography to afford the title compound. ¹H NMR (400 MHz, CD₃OD) δ=3.99-3.87 (m, 1H), 3.24-3.17 (m, 1H), 2.83-2.77 (m, 1H), 2.58-2.52 (m, 1H), 2.46-2.39 (m, 1H), 2.22-2.11 (m, 1H), 2.02-1.94 (m, 1H), 1.83-1.64 (m, 6H), 1.59 (dd, J=7.6, 14.7 Hz, 1H), 1.42-1.39 (m, 9H).

### Step 4: 1-(tert-butylsulfonyl)-1-azaspiro[4.4]nonan-3-ol

To a solution of 2-methyl-N-(1-(oxiran-2-ylmethyl)cyclopentyl)propane-2-sulfonamide (30 g, 114.78 mmol) in DMF (150 mL) was added potassium iodide (19.05 g, 114.78 mmol) and potassium carbonate (31.73 g, 229.55 mmol) at 20° C. After stirred at 100° C. for 3 h, the reaction mixture was diluted with water (1000 mL) and extracted with ethyl acetate (250 mL×4). The combined organic layers were washed with brine (500 mL), dried over anhydrous sodium sulfate, filtered and the filtrate was concentrated under reduced pressure. The residue was purified by column chromatography to afford the title compound. ¹H NMR (400 MHz, CD₃OD) δ=4.38-4.29 (m, 1H), 3.73 (dd, J=6.4, 10.1 Hz, 1H), 3.37-3.26 (m, 1H), 2.48-2.23 (m, 2H), 2.21-2.11 (m, 1H), 1.89-1.74 (m, 4H), 1.67-1.50 (m, 3H), 1.39 (s, 9H).

### Step 5: 1-(tert-butylsulfonyl)-1-azaspiro[4.4]nonan-3-one

To a solution of 1-(tert-butylsulfonyl)-1-azaspiro[4.4]nonan-3-ol (10 g, 38.26 mmol) in dichloromethane (100 mL) was added dess-martin periodinane (24.34 g, 57.39 mmol) at 20° C. After stirred at 20° C. for 12 h, the mixture was quenched by addition of saturated aqueous sodium thiosulfate (100 mL) and saturated aqueous sodium hydrogencarbonate (100 mL) extracted with dichloromethane (100 mL×4). The combined organic layers were washed with brine (100 mL), dried over anhydrous sodium sulfate, filtered and the filtrate was concentrated under reduced pressure. The residue was purified by column chromatography to afford the title compound. ¹H NMR (400 MHz, CD₃OD) δ=3.94-3.87 (m, 2H), 2.60-2.47 (m, 4H), 1.96-1.84 (m, 2H), 1.79-1.69 (m, 2H), 1.62-1.51 (m, 2H), 1.43 (s, 9H).

### Step 6: 1-(1-(tert-butylsulfonyl)-1-azaspiro[4.4]nonan-3-yl)-6-chloro-1,2,3,4-tetrahydroquinoline

To a solution of 1-(tert-butylsulfonyl)-1-azaspiro[4.4]nonan-3-one (2.5 g, 9.64 mmol) in acetic acid (80 mL) was added 6-chloro-1,2,3,4-tetrahydroquinoline (1.62 g, 9.64 mmol) and sodium triacetoxyborohydride (6.13 g, 28.92 mmol) at 20° C. After stirred at 20° C. for 12 h, the mixture was quenched by addition of saturated aqueous sodium bicarbonate (150 mL) and extracted with ethyl acetate (50 mL×4). The combined organic layers were washed with brine (500 mL), dried over anhydrous sodium sulfate, filtered and the filtrate was concentrated under reduced pressure. The residue was purified by column chromatography to afford the title compound. LCMS R_{T}=0.672 min, m/z=411.5 [M+H]⁺.

### Step 7: 8-bromo-1-(1-(tert-butylsulfonyl)-1-azaspiro[4.4]nonan-3-yl)-6-chloro-1,2,3,4-tetrahydroquinoline

To a solution of 1-(1-(tert-butylsulfonyl)-1-azaspiro[4.4]nonan-3-yl)-6-chloro-1,2,3,4-tetrahydroquinoline (480 mg, 1.17 mmol) in DMF (4 mL) was added 1-bromopyrrolidine-2,5-dione (208 mg, 1.17 mmol) at 20° C. After stirred at 20° C. for 1 h, the reaction mixture was diluted with water (50 mL) and extracted with ethyl acetate (10 mL×4). The combined organic layers were washed with brine (10 mL), dried over anhydrous sodium sulfate, filtered and the filtrate was concentrated under reduced pressure. The residue was purified by column chromatography to afford the title compound. ¹H NMR (400 MHz, CD₃OD) δ=7.43-7.36 (m, 1H), 7.07-7.01 (m, 1H), 4.34-4.22 (m, 1H), 3.96-3.87 (m, 1H), 3.27-3.22 (m, 1H), 3.10-3.01 (m, 1H), 2.82-2.73 (m, 2H), 2.58-2.47 (m, 1H), 2.30-2.19 (m, 1H), 2.07-2.01 (m, 2H), 1.85-1.74 (m, 4H), 1.71-1.45 (m, 5H), 1.41-1.40 (m, 9H). LCMS R_{T}=3.055 min, m/z=491.2 [M+H]⁺.

### Step 8: 2-(((tert-butyldimethylsilyl)oxy)methyl)-7-(1-(1-(tert-butylsulfonyl)-1-azaspiro[4.4]nonan-3-yl)-6-chloro-1,2,3,4-tetrahydroquinolin-8-yl)thieno[3,2-b]pyridine

To a solution of 8-bromo-1-(1-(tert-butylsulfonyl)-1-azaspiro[4.4]nonan-3-yl)-6-chloro-1,2,3,4-tetrahydroquinoline (300 mg, 0.61 mmol) in water (0.5 mL) and dioxane (5 mL) was added (2-(((tert-butyldimethylsilyl)oxy)methyl)thieno[3,2-b]pyridin-7-yl)boronic acid (396 mg, 1.22 mmol), dichloro[1,1′-bis(diphenylphosphino)ferrocene]palladium(II) (67 mg, 0.092 mmol) and dipotassium carbonate (254 mg, 1.84 mmol) at 20° C. under nitrogen atmosphere. After stirred at 100° C. for 3 h, the reaction mixture was filtered and the filtrate was concentrated under reduced pressure to remove the solvent. The residue was purified by column chromatography to afford the title compound. LCMS R_{T}=0.678 min, m/z=688.4 [M+H]⁺.

### Step 9: 8-(2-(((tert-butyldimethylsilyl)oxy)methyl)thieno[3,2-b]pyridin-7-yl)-1-(1-(tert-butylsulfonyl)-1-azaspiro[4.4]nonan-3-yl)-1,2,3,4-tetrahydroquinoline-6-carbonitrile

To a solution of 2-(((tert-butyldimethylsilyl)oxy)methyl)-7-(1-(1-(tert-butylsulfonyl)-1-azaspiro[4.4]nonan-3-yl)-6-chloro-1,2,3,4-tetrahydroquinolin-8-yl)thieno[3,2-b]pyridine (800 mg, 1.16 mmol) in 1-methylpyrrolidin-2-one (10 mL) was added bis(tri-tert-butylphosphine)palladium(0) (119 mg, 0.23 mmol) and zinc cyanide (2.32 g, 19.76 mmol) at 20° C. under nitrogen atmosphere. After stirred at 170° C. for 1 h in a microwave oven, the reaction mixture was diluted with water (100 mL) and extracted with ethyl acetate (20 mL×4). The combined organic layers were washed with brine (20 mL), dried over anhydrous sodium sulfate, filtered and the filtrate was concentrated under reduced pressure. The residue was purified by column chromatography to afford the title compound. The residue was purified by column chromatography to afford the title compound. LCMS R_{T}=0.633 min, m/z=679.9 [M+H]⁺.

### Step 10: (R)-1-(1-(tert-butylsulfonyl)-1-azaspiro[4.4]nonan-3-yl)-8-(2-(hydroxymethyl)thieno[3,2-b]pyridin-7-yl)-1,2,3,4-tetrahydroquinoline-6-carbonitrile and (S)-1-(1-(tert-butylsulfonyl)-1-azaspiro[4.4]nonan-3-yl)-8-(2-(hydroxymethyl)thieno[3,2-b]pyridin-7-yl)-1,2,3,4-tetrahydroquinoline-6-carbonitrile

To a solution of 8-(2-(((tert-butyldimethylsilyl)oxy)methyl)thieno[3,2-b]pyridin-7-yl)-1-(1-(tert-butylsulfonyl)-1-azaspiro[4.4]nonan-3-yl)-1,2,3,4-tetrahydroquinoline-6-carbonitrile (780 mg, 1.15 mmol) in THF (1 mL) was added tetrabutylammonium fluoride (1 M in THF, 1 mmol) at 15° C. After stirred at 15° C. for 0.5 h, the reaction mixture was concentrated under reduced pressure to remove solvent. The residue was purified by column chromatography. The racemate was separated by SFC to give first eluting fraction (178.1, Rt=1.805; LCMS R_{T}=0.897 min, m/z=565.1 [M+H]+) and second eluting fraction (178.2, Rₜ=2.356; LCMS R_{T}=0.897 min, m/z=565.1 [M+H]+).

### Step 11

Example 178a was prepared from 178.1 and 3-azabicyclo[3.1.0]hexane-2,4-dione, following the procedure described in the synthesis of Example 108. The product was purified by preparative HPLC. ¹H NMR (400 MHz, CD₃OD) δ=8.82-8.70 (m, 1H), 8.60-8.46 (m, 1H), 7.57-7.39 (m, 4H), 4.82-4.79 (m, 2H), 3.96-3.72 (m, 1H), 3.10-2.76 (m, 3H), 2.71-2.50 (m, 3H), 2.09-1.65 (m, 5H), 1.65-1.47 (m, 6H), 1.46-1.29 (m, 2H), 1.24-0.32 (m, 3H). LCMS: R_{T}=0.433 min, m/z=538.3 [M+H]⁺.

Example 178b was prepared from 178.2 and 3-azabicyclo[3.1.0]hexane-2,4-dione, following the procedure described in the synthesis of Example 108. The product was purified by preparative HPLC. ¹H NMR (400 MHz, CD₃OD) δ=8.82-8.70 (m, 1H), 8.60-8.46 (m, 1H), 7.57-7.39 (m, 4H), 4.82-4.79 (m, 2H), 3.96-3.72 (m, 1H), 3.10-2.76 (m, 3H), 2.71-2.50 (m, 3H), 2.09-1.65 (m, 5H), 1.65-1.47 (m, 6H), 1.46-1.29 (m, 2H), 1.24-0.32 (m, 3H). LCMS: R_{T}=0.437 min, m/z=538.3 [M+H]⁺.

The absolute configuration was tentatively assigned based on the relative potency of the pair diastereomers in biology assays.

### Example 179a and 179b: 1-((7-(6-chloro-1-((5S,7R)-1-azaspiro[4.4]nonan-7-yl)-1,2,3,4-tetrahydroquinolin-8-yl)thieno[3,2-b]pyridin-2-yl)methyl)pyrrolidine-2,5-dione, formic acid salt and 1-((7-(6-chloro-1-((5R,7S)-1-azaspiro[4.4]nonan-7-yl)-1,2,3,4-tetrahydroquinolin-8-yl)thieno[3,2-b]pyridin-2-yl)methyl)pyrrolidine-2,5-dione, formic acid salt

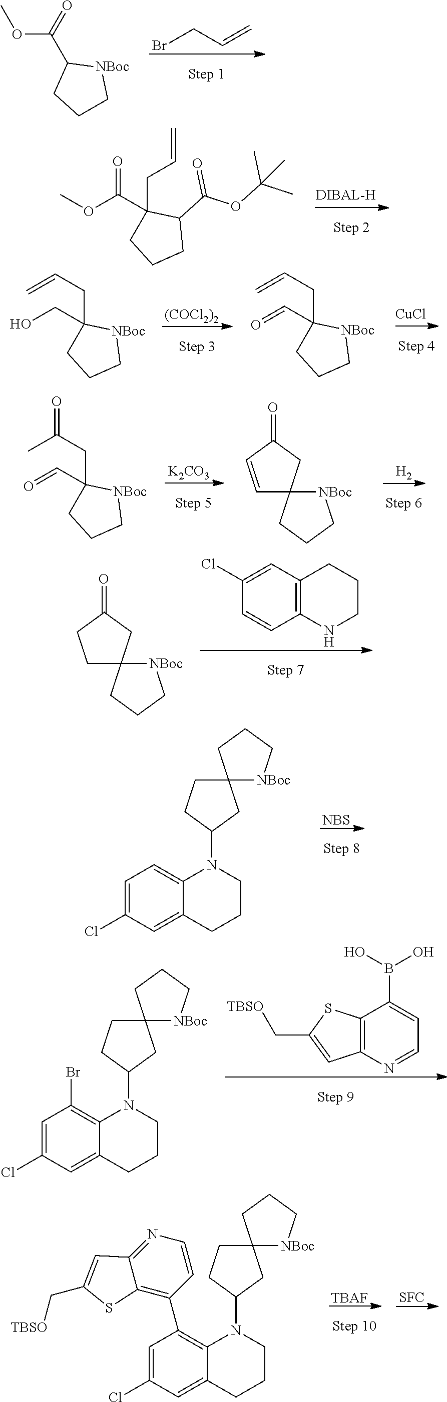

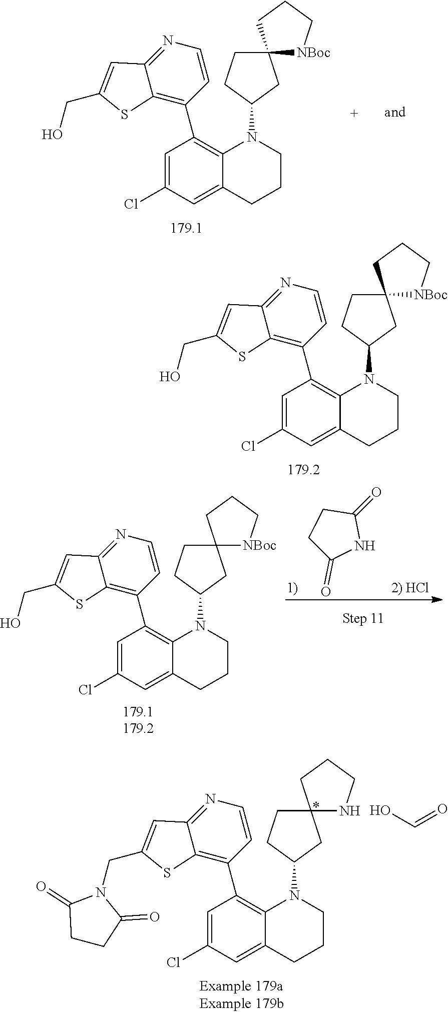

### Step 1: 1-tert-butyl 2-methyl 2-allylpyrrolidine-1,2-dicarboxylate

To a solution of 1-tert-butyl 2-methyl pyrrolidine-1,2-dicarboxylate (50 g, 218.08 mmol) in THF (100 mL) was added [bis(trimethylsilyl)amino]lithium (261.70 mmol, 1M in THF) at −78° C. The mixture was stirred at −78° C. for 0.5 h, then the mixture 3-bromoprop-1-ene (39.57 g, 327.12 mmol) in THF (2 mL) was stirred at −78° C. After stirred at −78° C. for 0.5 h, the mixture was quenched by addition of saturated aqueous ammonium chloride (60 mL) and extracted with ethyl acetate (40 mL×3). The combined organic layers were washed with brine (40 mL), dried over anhydrous sodium sulfate, filtered and the filtrate was concentrated under reduced pressure. The residue was purified by column chromatography to afford the title compound. ¹H NMR (400 MHz, CDCl₃) δ=5.68-5.51 (m, 1H), 5.02-4.81 (m, 2H), 3.54-3.35 (m, 1H), 3.28-3.14 (m, 1H), 2.76 (dd, J=14.4 Hz, 6.4 Hz, 1H), 2.44 (dd, J=14.4 Hz, 8.0 Hz, 1H), 2.02-1.83 (m, 3H), 1.76-1.60 (m, 2H), 1.31-1.29 (m, 1H), 1.32-1.28 (m, 3H), 1.26 (s, 7H).

### Step 2: tert-butyl 2-allyl-2-(hydroxymethyl)pyrrolidine-1-carboxylate

To a solution of 1-tert-butyl 2-methyl 2-allylpyrrolidine-1,2-dicarboxylate (48 g, 178.22 mmol) in THF (20 mL) was added bis(2-methylpropyl)aluminum hydride (535 mL, 1 M in THF) at −78° C. After stirred at −78° C. for 2 h, the reaction mixture was quenched by addition of saturated aqueous ammonium chloride (100 mL) and extracted with ethyl acetate (50 mL×3). The combined organic layers were washed with brine (80 mL), dried over anhydrous sodium sulfate, filtered and the filtrate was concentrated under reduced pressure. The residue was purified by column chromatography to afford the title compound. LCMS R_{T}=0.942 min, m/z=186.2 [M+H−56]⁺.

### Step 3: tert-butyl 2-allyl-2-formyl-pyrrolidine-1-carboxylate

To a solution of dimethyl sulfoxide (66.05 g, 845.33 mmol) in dichloromethane (10 mL) was added oxalyl dichloride (53.65 g, 422.67 mmol) in dichloromethane (10 mL) at −78° C. After stirred at −78° C. for 0.5 h, the solution of tert-butyl 2-allyl-2-(hydroxymethyl)pyrrolidine-1-carboxylate (34 g, 140.89 mmol) in dichloromethane (10 mL) was dropwise added at −78° C. After stirred at −78° C. for 0.5 h, N,N-diethylethanamine (71.28 g, 704.44 mmol) was dropwise added to the reaction mixture at −78° C. for 0.5 h and the reaction mixture was stirred at 20° C. for 1.5 h. The reaction mixture was diluted with water (200 mL) and extracted with dichloromethane (80 mL×3). The combined organic layers were washed with brine (80 mL), dried over anhydrous sodium sulfate, filtered and the filtrate was concentrated under reduced pressure. The residue was purified by column chromatography to afford the title compound. LCMS R_{T}=0.941 min, m/z=184.2 [M+H−56]⁺.

### Step 4: tert-butyl 2-formyl-2-(2-oxopropyl)pyrrolidine-1-carboxylate

To a solution of tert-butyl 2-allyl-2-formyl-pyrrolidine-1-carboxylate (20 g, 83.57 mmol) in DMF (70 mL) and water (10 mL) was added dichloropalladium (2.22 g, 12.54 mmol) and copper(I) chloride (8.27 g, 83.57 mmol) at 20° C. under oxygen. After stirred at 25° C. for 16 h, the reaction mixture was diluted with water (100 mL) and extracted with ethyl acetate (50 mL×3). The combined organic layers were washed with brine (50 mL), dried over anhydrous sodium sulfate, filtered and the filtrate was concentrated under reduced pressure to give a residue. The residue was purified by column chromatography to afford the title compound. LCMS R_{T}=0.840 min, m/z=200.2 [M+H−56]⁺.

### Step 5: tert-butyl 8-oxo-1-azaspiro[4.4]non-6-ene-1-carboxylate

To a solution of tert-butyl 2-formyl-2-(2-oxopropyl)pyrrolidine-1-carboxylate (20 g, 78.34 mmol) in methanol (50 mL) and water (50 mL) was added the mixture of dipotassium carbonate (16.24 g, 117.50 mmol) in water (50 mL) at 20° C. After stirred at 70° C. for 1 h, the reaction mixture was diluted with saturated aqueous ammonium chloride (100 mL) and extracted with dichloromethane (50 mL×3). The combined organic layers were washed with brine (15 mL), dried over anhydrous sodium sulfate, filtered and the filtrate was concentrated under reduced pressure to give a residue. The residue was purified by column chromatography to afford the title compound. LCMS R_{T}=0.811 min, m/z=182.2 [M+H−56]⁺.

### Step 6: tert-butyl 7-oxo-1-azaspiro[4.4]nonane-1-carboxylate

To a solution of tert-butyl 8-oxo-1-azaspiro[4.4]non-6-ene-1-carboxylate (8.5 g, 35.82 mmol) in ethanol (100 mL) was added palladium on carbon (900 mg, 35.82 mmol, 10% purity) at 20° C. After stirred at 25° C. for 12 h, the reaction mixture was filtered and the filtrate was concentrated under reduced pressure to give a residue. The residue was purified by column chromatography to afford the title compound. LCMS R_{T}=1.147 min, m/z=184.2 [M+H−56]⁺.

### Step 7: tert-butyl 7-(6-chloro-3,4-dihydroquinolin-1(2H)-yl)-1-azaspiro[4.4]nonane-1-carboxylate

To a solution of tert-butyl 7-oxo-1-azaspiro[4.4]nonane-1-carboxylate (6.4 g, 26.74 mmol) in acetic acid (5 mL) was added 6-chloro-1,2,3,4-tetrahydroquinoline (6.58 g, 29.42 mmol) and sodium triacetoxyborohydride (11.34 g, 53.49 mmol) at 20° C. After stirred at 20° C. for 1 h, The reaction mixture was quenched by addition of saturated aqueous sodium hydrogen carbonate (300 mL) and extracted with dichloromethane (30 mL×3). The combined organic layers were washed with brine (15 mL), dried over anhydrous sodium sulfate, filtered and the filtrate was concentrated under reduced pressure. The residue was purified by column chromatography to afford the title compound. LCMS R_{T}=2.657 min, m/z=391.0 [M+H]⁺.

### Step 8: tert-butyl 7-(6-chloro-3,4-dihydroquinolin-1(2H)-yl)-1-azaspiro[4.4]nonane-1-carboxylate

To a solution of tert-butyl 8-(6-chloro-3,4-dihydro-2H-quinolin-1-yl)-1-azaspiro[4.4]nonane-1-carboxylate (400 mg, 1.02 mmol) in dichloromethane (5 mL) was added 1-bromopyrrolidine-2,5-dione (1.76 g, 9.87 mmol) in dichloromethane (4 mL) at 0° C. After stirred at 20° C. for 1 h, then the reaction mixture was concentrated under reduced pressure to remove solvent. The residue was purified by column chromatography to afford the title compound. LCMS R_{T}=0.901 min, m/z=471.2 [M+H]⁺.

### Step 9: tert-butyl 7-(8-(2-(((tert-butyldimethylsilyl)oxy)methyl)thieno[3,2-b]pyridin-7-yl)-6-chloro-3,4-dihydroquinolin-1(2H)-yl)-1-azaspiro[4.4]nonane-1-carboxylate

To a solution of tert-butyl 7-(6-chloro-3,4-dihydroquinolin-1(2H)-yl)-1-azaspiro[4.4]nonane-1-carboxylate (200 mg, 0.43 mmol) in water (0.4 mL) and dioxane (2 mL) was added (2-(((tert-butyldimethylsilyl)oxy)methyl)thieno[3,2-b]pyridin-7-yl)boronic acid (138 mg, 0.43 μmo), bis(tri-tert-butylphosphine)palladium(0) (95 mg, 0.13 mmol) and dipotassium carbonate (177 mg, 1.28 mmol) at 20° C. under nitrogen atmosphere. After stirred at 100° C. for 2 h, the reaction mixture was filtered and the filtrate was concentrated under reduced pressure to remove the solvent. The residue was purified by column chromatography to afford the title compound. LCMS R_{T}=1.134 min, m/z=668.2 [M+H]⁺.

### Step 10: tert-butyl (5R,7R)-7-(6-chloro-8-(2-(hydroxymethyl)thieno[3,2-b]pyridin-7-yl)-3,4-dihydroquinolin-1(2H)-yl)-1-azaspiro[4.4]nonane-1-carboxylate and tert-butyl (5S,7S)-7-(6-chloro-8-(2-(hydroxymethyl)thieno[3,2-b]pyridin-7-yl)-3,4-dihydroquinolin-1(2H)-yl)-1-azaspiro[4.4]nonane-1-carboxylate

To a solution of tert-butyl 7-(8-(2-(((tert-butyldimethylsilyl)oxy)methyl)thieno[3,2-b]pyridin-7-yl)-6-chloro-3,4-dihydroquinolin-1(2H)-yl)-1-azaspiro[4.4]nonane-1-carboxylate (250 mg, 0.37 mmol) in THF (2 mL) was added tetrabutylammonium fluoride (2 mL, 1 M in THF). After stirred at 20° C. for 0.5 h, the reaction mixture was concentrated under reduced pressure to remove solvent. The residue was purified by column chromatography. The racemate was separated by SFC to give first eluting fraction (179.1, R₁=2.059 min; LCMS R_{T}=0.929 min, m/z=554.0 [M+H]+) and second eluting fraction (179.2, Rt=2.479 min; LCMS R_{T}=0.933 min, m/z=554.1 [M+H]+).

### Step 11

Example 179a was prepared from 179.1 and succinimide, following the procedure described in the synthesis of Example 54. The product was purified by preparative HPLC. ¹H NMR (400 MHz, CD₃OD) δ=8.68 (d, J=4.8 Hz, 1H), 8.52 (s, 1H), 7.51-7.34 (m, 2H), 7.10 (dd, J=17.6 Hz, 2.4 Hz, 2H), 4.94 (s, 2H), 3.28-3.02 (m, 5H), 2.85 (t, J=6.4 Hz, 2H), 2.76 (s, 4H), 1.96-1.18 (m, 11H), 0.88 (s, 1H). LCMS R_{T}=0.741 min, m/z=535.1 [M+H]⁺. *Absolute configuration was not determined.

Example 179b was prepared from 179.2 and succinimide, following the procedure described in the synthesis of Example 54. The product was purified by preparative HPLC. ¹H NMR (400 MHz, CD₃OD) δ=8.65 (d, J=4.8 Hz, 1H), 8.50 (s, 1H), 7.48-7.30 (m, 2H), 7.07 (dd, J=17.6 Hz, 2.4 Hz, 2H), 4.92 (s, 2H), 3.25-2.99 (m, 5H), 2.82 (t, J=6.4 Hz, 2H), 2.74 (s, 4H), 2.04-1.06 (m, 11H), 0.85 (s, 1H). LCMS R_{T}=0.741 min, m/z=535.1 [M+H]⁺. *Absolute configuration was not determined.

### Example 180: 1-((7-(6-chloro-1-(5-azaspiro[3.4]octan-2-yl)-1,2,3,4-tetrahydroquinolin-8-yl)thieno[3,2-b]pyridin-2-yl)methyl)pyrrolidine-2,5-dione, formic acid salt

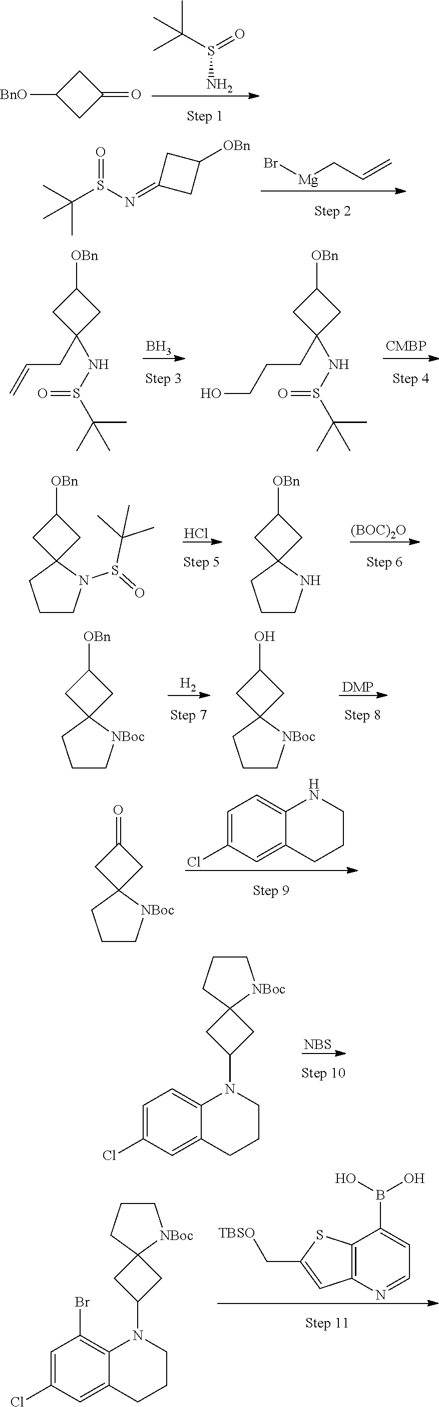

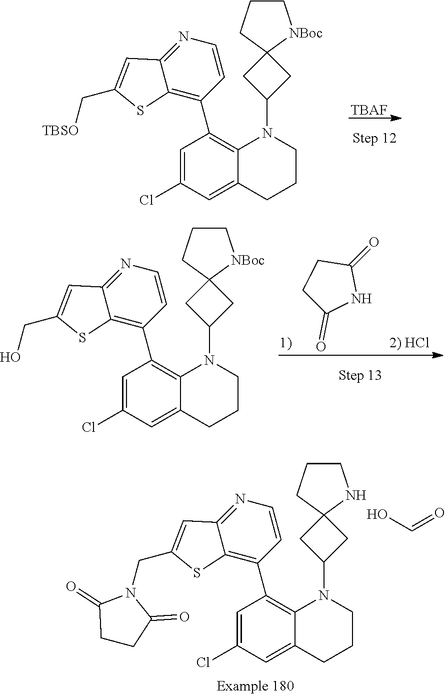

### Step 1: N-(3-(benzyloxy)cyclobutylidene)-2-methylpropane-2-sulfinamide

To a solution of 3-(benzyloxy)cyclobutanone (8.8 g, 49.94 mmol) in THF (80 mL) was added (S)-2-methylpropane-2-sulfinamide (6.7 g, 54.93 mmol) and tetraisopropoxytitanium (16.9 g, 59.43 mmol) at 20° C. After stirred at 50° C. for 12 h, the reaction mixture was quenched by addition saturated sodium chloride (50 mL) at 0° C. and extracted with ethyl acetate (40 mL×2). The combined organic layers were concentrated directly. The residue was purified by column chromatography to afford the title compound. LCMS R_{T}=0.671 min, m/z=280.5 [M+H]⁺.

### Step 2: N-(1-allyl-3-(benzyloxy)cyclobutyl)-2-methylpropane-2-sulfinamide

To a solution of N-(3-(benzyloxy)cyclobutylidene)-2-methylpropane-2-sulfinamide (21.8 g, 77.85 mmol) in THF (200 mL) was added allylmagnesium bromide (116.77 mL, 1M in THF) at −78° C. After stirred at −78° C. for 1 h, the reaction mixture was quenched by addition of ammonium chloride (100 mL) and extracted with ethyl acetate (100 mL×3). The combined organic layers were washed with brine (300 mL), dried over anhydrous sodium sulfate and concentrated under reduced pressure. The residue was purified by column chromatography to afford the title compound. LCMS R_{T}=1.367 min, m/z=322.2[M+H]⁺.

### Step 3: N-(3-(benzyloxy)-1-(3-hydroxypropyl)cyclobutyl)-2-methylpropane-2-sulfinamide

To a solution of N-(1-allyl-3-(benzyloxy)cyclobutyl)-2-methylpropane-2-sulfinamide (12.1 g, 37.73 mmol) in THF (120 mL) was added borane (94.33 mL, 1M in THF) at 0° C. After stirred at 20° C. for 1 h, the reaction mixture was added hydrogen peroxide (97 g, 855.87 mmol) and sodium hydroxide (94.33 mL, 2M in water) at 0° C. After stirred at 20° C. for 1 h, the reaction mixture was quenched by addition of sodium thiosulfate (50 mL) at 0° C. and extracted with methylene chloride (60 mL×3). The combined organic layers were washed with brine (180 mL), dried over anhydrous sodium sulfate and concentrated under reduced pressure. The residue was purified by column chromatography to afford the title compound. LCMS R_{T}=0.609 min, m/z=340.1[M+H]⁺.

### Step 4: 2-(benzyloxy)-5-(tert-butylsulfinyl)-5-azaspiro[3.4]octane

To a solution of N-(3-(benzyloxy)-1-(3-hydroxypropyl)cyclobutyl)-2-methylpropane-2-sulfinamide (4.3 g, 12.58 mmol) in toluene (50 mL) was added 2-(tributyl-,5-phosphanylidene) acetonitrile (4.6 g, 18.87 mmol) at 20° C. After stirred at 110° C. for 1 h, the reaction mixture was concentrated under reduced pressure to remove solvent. The residue was purified by column chromatography to afford the title compound. LCMS R_{T}=0.717 min, m/z=322.2[M+H]⁺.

### Step 5: 2-(benzyloxy)-5-azaspiro[3.4]octane

To a solution of 2-(benzyloxy)-5-(tert-butylsulfinyl)-5-azaspiro[3.4]octane (2 g, 6.22 mmol) was dissolved in dioxane (10 mL) and added hydrochloric acid (10 mL, 4 M in dioxane). After stirred at 25° C. for 30 min, the reaction mixture was concentrated under reduced pressure to afford the title compound. It was used directly and without further purification in the next step. LCMS R_{T}=0.547 min, m/z=218.3 [M+H]⁺.

### Step 6: tert-butyl 2-(benzyloxy)-5-azaspiro[3.4]octane-5-carboxylate

To a solution of 2-(benzyloxy)-5-azaspiro[3.4]octane (1.4 g, 6.21 mmol) in dichloromethane (30 mL) was added dimethylaminopyridine (76 mg, 0.62 mmol), triethylamine (1.9 g, 18.64 mmol) and tert-butyldicarbonate (2 g, 9.32 mmol). After stirred at 25° C. for 1 h, the reaction mixture was concentrated under reduced pressure to give a residue. The residue was purified by column chromatography to afford the title compound. LCMS R_{T}=1.508 min, m/z=262.3 [M+H−56]⁺.

### Step 7: tert-butyl 2-hydroxy-5-azaspiro[3.4]octane-5-carboxylate

To a solution of tert-butyl 2-(benzyloxy)-5-azaspiro[3.4]octane-5-carboxylate (2 g, 6.30 mmol) in ethyl alcohol (10 mL) was added Hydroxide palladium (885 mg, 6.30 mmol). After stirred at 60° C. for 3 h under hydrogen atmosphere, the reaction mixture was filtered by suction filtration and the filtrate was concentrated under reduced pressure to afford the title compound. It was used directly and without further purification in the next step. LCMS R_{T}=0.900 min, m/z=172.2 [M+H−56]⁺.

### Step 8: tert-butyl 2-oxo-5-azaspiro[3.4]octane-5-carboxylate

To a solution of tert-butyl 2-hydroxy-5-azaspiro[3.4]octane-5-carboxylate (1.3 g, 5.72 mmol) in dimethylaminopyridine (40 mL) was added Dess-Martin Oxidation (3.6 g, 8.58 mmol). After stirred at 25° C. for 1 h, the reaction mixture was filtered and the filtrate was concentrated under reduced pressure to give a residue. The residue was purified by column chromatography to afford the title compound. ¹H NMR (400 MHz, CDCl₃) δ=4.04-3.99 (m, 1H), 3.73-3.68 (m, 1H), 3.56-3.46 (m, 2H), 3.00-2.95 (m, 1H), 2.84-2.80 (m, 1H), 2.25-2.13 (m, 2H), 1.84-1.81 (m, 2H), 1.48 (s, 9H).

### Step 9: tert-butyl 2-(6-chloro-3,4-dihydroquinolin-1(2H)-yl)-5-azaspiro[3.4]octane-5-carboxylate

To a solution of 6-chloro-1,2,3,4-tetrahydroquinoline (880 mg, 5.25 mmol) and tert-butyl 2-oxo-5-azaspiro[3.4]octane-5-carboxylate (1.1 g, 4.88 mmol) in acetic acid (20 mL) was added sodium triacetoborohydride (2.1 g, 9.77 mmol). After stirred at 25° C. for 1 h, the reaction mixture was concentrated under reduced pressure. The residue was purified by column chromatography to afford the title compound. LCMS R_{T}=1.377 min, m/z=377.3 [M+H]⁺.

### Step 10: tert-butyl 2-(8-bromo-6-chloro-3,4-dihydroquinolin-1(2H)-yl)-5-azaspiro[3.4]octane-5-carboxylate

To a solution of tert-butyl 2-(6-chloro-3,4-dihydroquinolin-1(2H)-yl)-5-azaspiro[3.4]octane-5-carboxylate (300 mg, 0.80 mmol) in DMF (10 mL) was added N-bromosuccinimide (283 mg, 1.59 mmol). After stirred at 0° C. for 0.5 h, the reaction mixture was concentrated under reduced pressure to remove the solvent. The residue was purified by column chromatography to afford the title compound. LCMS R_{T}=1.583 min, m/z=457.3 [M+H]⁺.

### Step 11: tert-butyl 2-(8-(2-(((tert-butyldimethylsilyl)oxy)methyl)thieno[3,2-b]pyridin-7-yl)-6-chloro-3,4-dihydroquinolin-1(2H)-yl)-5-azaspiro[3.4]octane-5-carboxylate

To a solution of tert-butyl 2-(8-bromo-6-chloro-3,4-dihydroquinolin-1(2H)-yl)-5-azaspiro[3.4]octane-5-carboxylate (200 mg, 0.44 mmol), (2-(((tert-butyldimethylsilyl)oxy)methyl)thieno[3,2-b]pyridin-7-yl)boronic acid (213 mg, 0.66 mmol) in dioxane (10 mL) and water (1 mL) was added 1,1-bis(diphenylphosphorus) ferrocene palladium chloride (64 mg, 0.09 mol) and cesium carbonate (429 mg, 1.32 mmol). After stirred at 100° C. for 2 h under nitrogen atmosphere, the reaction mixture was filtered by suction filtration and the filtrate was concentrated under reduced pressure. The residue was purified by column chromatography to afford the title compound. LCMS R_{T}=1.178 min, m/z=654.5 [M+H]⁺.

### Step 12: tert-butyl 2-(6-chloro-8-(2-(hydroxymethyl)thieno[3,2-b]pyridin-7-yl)-3,4-dihydroquinolin-1(2H)-yl)-5-azaspiro[3.4]octane-5-carboxylate

To a solution of tert-butyl 2-(8-(2-(((tert-butyldimethylsilyl)oxy)methyl)thieno[3,2-b]pyridin-7-yl)-6-chloro-3,4-dihydroquinolin-1(2H)-yl)-5-azaspiro[3.4]octane-5-carboxylate (110 mg, 0.17 mmol) in THF (5 mL) was added tetrabutylammonium fluoride (88 mg, 0.34 mmol). After stirred at 25° C. for 1 h, the reaction mixture was concentrated under reduced pressure. The residue was purified by column chromatography to afford the title compound. LCMS R_{T}=2.422 min, m/z=540.4 [M+H]⁺.

### Step 13

Example 180 was prepared from tert-butyl 2-(6-chloro-8-(2-(hydroxymethyl)thieno[3,2-b]pyridin-7-yl)-3,4-dihydroquinolin-1(2H)-yl)-5-azaspiro[3.4]octane-5-carboxylate and succinimide, following the procedure described in the synthesis of Example 54. The product was purified by preparative HPLC. ¹H NMR (400 MHz, CD₃OD) δ=8.68 (d, J=4.8 Hz, 1H), 8.48 (s, 1H), 7.45 (s, 1H), 7.42 (d, J=4.8 Hz, 1H), 7.16-7.12 (m, 2H), 4.94 (s, 2H), 3.27-3.13 (m, 4H), 3.05 (t, J=7.6 Hz, 2H), 2.88 (t, J=6.4 Hz, 2H), 2.20 (s, 4H), 1.83-1.72 (m, 7H), 1.37 (t, J=6.8 Hz, 2H). LCMS R_{T}=1.319 min, m/z=521.3 [M+H]⁺.

### Example 181a and 181b: (S)-1-((7-(1-(5,5-bis(hydroxymethyl)pyrrolidin-3-yl)-6-chloro-1,2,3,4-tetrahydroquinolin-8-yl)thieno[3,2-b]pyridin-2-yl)methyl)pyrrolidine-2,5-dione and (R)-1-((7-(1-(5,5-bis(hydroxymethyl)pyrrolidin-3-yl)-6-chloro-1,2,3,4-tetrahydroquinolin-8-yl)thieno[3,2-b]pyridin-2-yl)methyl)pyrrolidine-2,5-dione

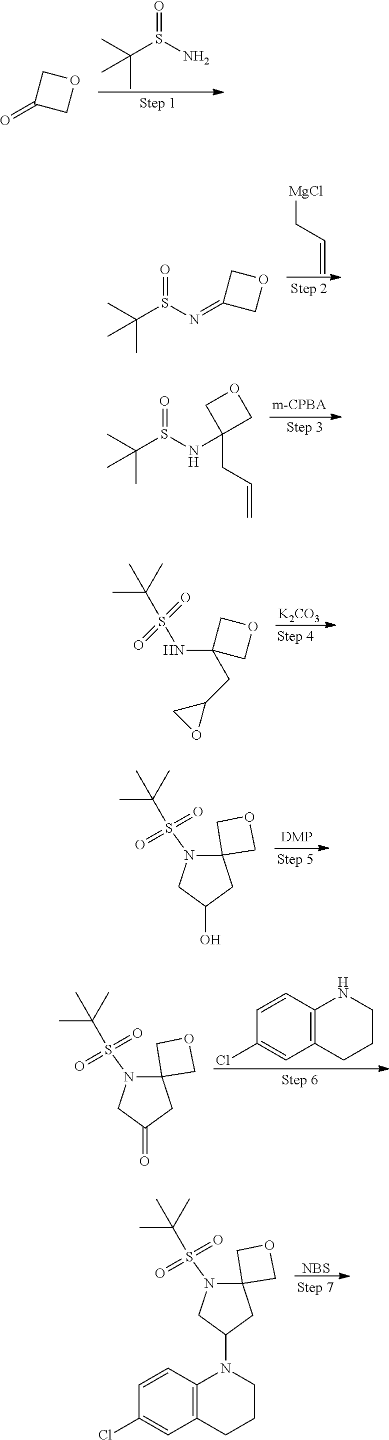

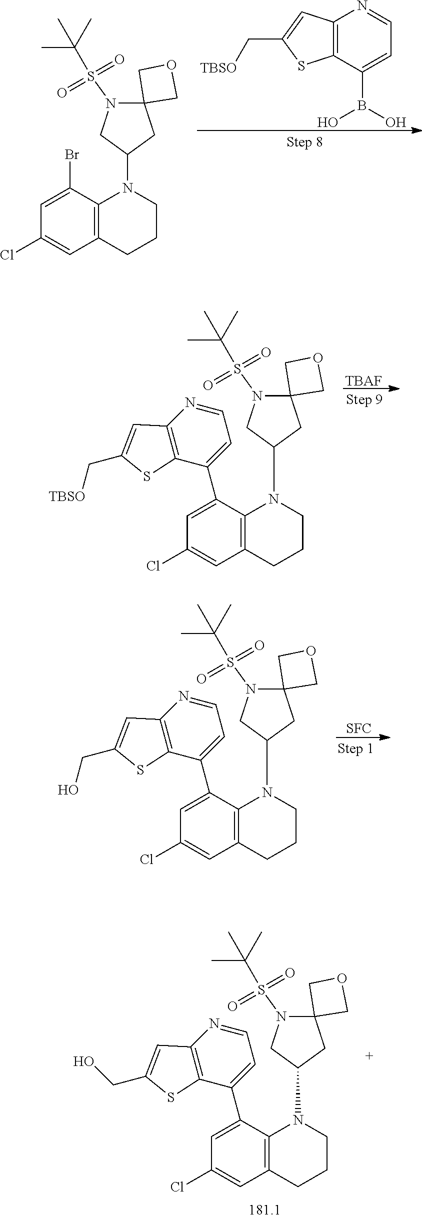

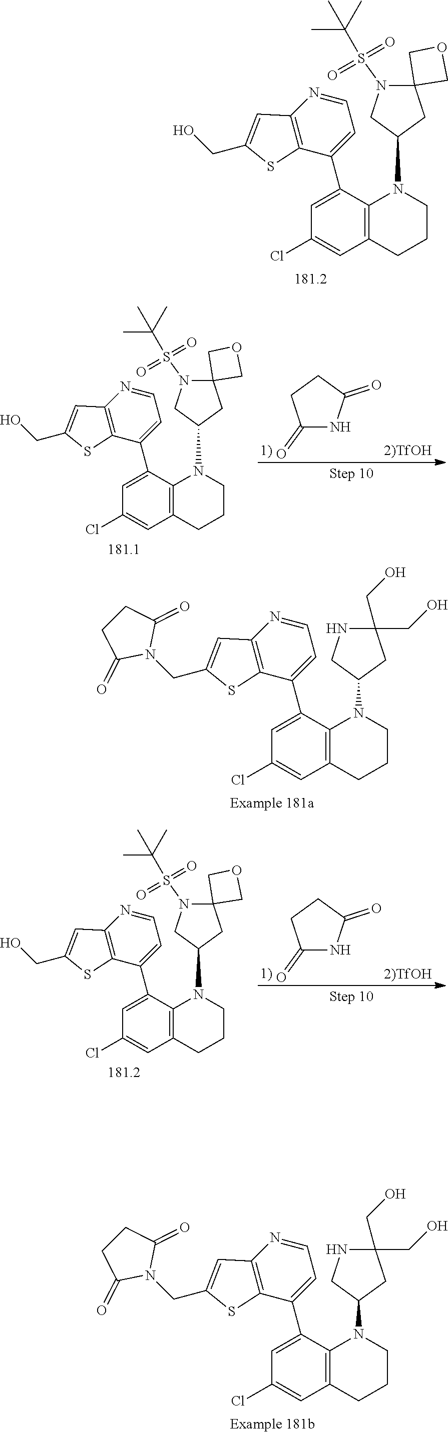

### Step 1: 2-methyl-N-(oxetan-3-ylidene)propane-2-sulfinamide

To a solution of oxetan-3-one (25 g, 346.92 mmol) in THF (100 mL) was added 2-methylpropane-2-sulfinamide (42.05 g, 346.92 mmol) and titanium(iv)isopropoxide (147.90 g, 520.38 mmol) at 20° C. After stirred at 20° C. for 1 h, the mixture was quenched by addition of saturated aqueous sodium hydrogen carbonate (30 mL) and filtered. The filtrate was diluted with water (20 mL) and extracted with ethyl acetate (50 mL×4). The combined organic layers were washed with brine (50 mL), dried over anhydrous sodium sulfate, filtered and the filtrate was concentrated under reduced pressure. The residue was purified by column chromatography to afford the title compound. ¹H NMR (400 MHz, CD₃OD) δ=5.84-5.75 (m, 1H), 5.69-5.60 (m, 1H), 5.52-5.37 (m, 2H), 1.26 (s, 9H).

### Step 2: N-(3-allyloxetan-3-yl)-2-methylpropane-2-sulfinamide

To a solution of 2-methyl-N-(oxetan-3-ylidene)propane-2-sulfinamide (18 g, 102.71 mmol) in THF (100 mL) was added allylmagnesium chloride (102.71 mL, 2 M in THF) at −78° C. After stirred at −78° C. for 2 h, the mixture was quenched by addition of saturated aqueous ammonium chloride (10 mL). The mixture was diluted with water (20 mL) and extracted with ethyl acetate (20 mL×4). The combined organic layers were washed with brine (20 mL), dried over anhydrous sodium sulfate, filtered and the filtrate was concentrated under reduced pressure. The residue was purified by column chromatography to afford the title compound. ¹H NMR (400 MHz, CD₃OD) δ=5.92 (td, J=8.0, 10.4, 17.2 Hz, 1H), 5.33-5.17 (m, 2H), 4.76 (dd, J=6.4, 9.2 Hz, 2H), 4.52 (dd, J=6.4, 16.8 Hz, 2H), 2.86-2.71 (m, 2H), 1.27 (s, 9H).

### Step 3: 2-methyl-N-(3-(oxiran-2-ylmethyl)oxetan-3-yl)propane-2-sulfonamide

To a solution of N-(3-allyloxetan-3-yl)-2-methylpropane-2-sulfinamide (11 g, 50.61 mmol) in dichloromethane (100 mL) was added 3-chlorobenzenecarboperoxoic acid (26.20 g, 151.84 mmol) at 20° C. After stirred at 20° C. for 1 h, the mixture was quenched by addition of saturated aqueous sodium thiosulfate (100 mL), saturated aqueous sodium hydrogencarbonate (100 mL) and extracted with dichloromethane (30 mL×4). The combined organic layers were washed with brine (20 mL), dried over anhydrous sodium sulfate, filtered and the filtrate was concentrated under reduced pressure. The residue was purified by column chromatography to afford the title compound. ¹H NMR (400 MHz, CD₃OD) δ=4.79 (dd, J=6.4, 14.4 Hz, 2H), 4.64 (d, J=6.4 Hz, 1H), 4.54 (d, J=6.4 Hz, 1H), 3.28-3.20 (m, 1H), 2.82 (t, J=4.8 Hz, 1H), 2.62 (dd, J=2.8, 5.2 Hz, 1H), 2.37 (dd, J=4.8, 14.4 Hz, 1H), 2.12 (dd, J=7.6, 14.8 Hz, 1H), 1.48-1.41 (m, 9H).

### Step 4: 5-(tert-butylsulfonyl)-2-oxa-5-azaspiro[3.4]octan-7-ol

To a solution of 2-methyl-N-(3-(oxiran-2-ylmethyl)oxetan-3-yl)propane-2-sulfonamide (7.3 g, 31.29 mmol) in DMF (20 mL) was added potassium iodide (5.19 g, 31.29 mmol) and potassium carbonate (10.81 g, 78.22 mmol) at 20° C. After stirred at 100° C. for 2 h, the reaction mixture was diluted with water (200 mL) and extracted with ethyl acetate (50 mL×4). The combined organic layers were washed with brine (50 mL), dried over anhydrous sodium sulfate, filtered and the filtrate was concentrated under reduced pressure. The residue was purified by column chromatography to afford the title compound. ¹H NMR (400 MHz, CD₃OD) δ=5.37 (d, J=6.0 Hz, 1H), 5.27 (d, J=5.6 Hz, 1H), 4.63 (d, J=5.6 Hz, 1H), 4.49 (d, J=6.0 Hz, 1H), 4.33 (tt, J=2.4, 4.4 Hz, 1H), 3.61 (dd, J=4.4, 10.4 Hz, 1H), 3.49 (d, J=10.4 Hz, 1H), 2.55 (d, J=12.8 Hz, 1H), 2.38 (dd, J=4.4, 12.8 Hz, 1H), 1.40 (s, 9H).

### Step 5: 5-(tert-butylsulfonyl)-2-oxa-5-azaspiro[3.4]octan-7-one

To a solution of 5-(tert-butylsulfonyl)-2-oxa-5-azaspiro[3.4]octan-7-ol (3 g, 12.03 mmol) in dichloromethane (20 mL) was added dess-martin periodinane (6.63 g, 15.64 mmol) at 20° C. After stirred at 20° C. for 8 h, the mixture was quenched by addition of saturated aqueous sodium hydrogencarbonate (30 mL), saturated aqueous sodium thiosulfate (30 mL) and extracted with dichloromethane (20 mL×4). The combined organic layers were washed with brine (20 mL), dried over anhydrous sodium sulfate, filtered and the filtrate was concentrated under reduced pressure. The residue was purified by column chromatography to afford the title compound. ¹H NMR (400 MHz, CD₃OD) δ=5.34 (d, J=6.8 Hz, 2H), 4.56 (d, J=6.8 Hz, 2H), 3.86 (s, 2H), 3.11 (s, 2H), 1.46 (s, 9H).

### Step 6: 5-(tert-butylsulfonyl)-7-(6-chloro-3,4-dihydroquinolin-1(2H)-yl)-2-oxa-5-azaspiro[3.4]octane

To a solution of 5-(tert-butylsulfonyl)-2-oxa-5-azaspiro[3.4]octan-7-one (900 mg, 3.64 mmol) in methanol (5 mL) was added 6-chloro-1,2,3,4-tetrahydroquinoline (732 mg, 4.37 mmol), triethyl silicane (1.67 g, 14.36 mmol) and indium(III) chloride (402.45 mg, 1.82 mmol) at 20° C. After stirred at 20° C. for 20 h, the mixture was filtered and the filtrate was concentrated under reduced pressure. The residue was purified by column chromatography to afford the title compound. LCMS R_{T}=0.608 min, m/z=399.1 [M+H]⁺.

### Step 7: 7-(8-bromo-6-chloro-3,4-dihydroquinolin-1(2H)-yl)-5-(tert-butylsulfonyl)-2-oxa-5-azaspiro[3.4]octane

To a solution of 5-(tert-butylsulfonyl)-7-(6-chloro-3,4-dihydroquinolin-1(2H)-yl)-2-oxa-5-azaspiro[3.4]octane (200 mg, 501.32 mmol) in DMF (1 mL) was added 1-bromopyrrolidine-2,5-dione (71.38 mg, 0.4 mmol) at 0° C. After stirred at 20° C. for 1 h, the mixture was diluted with water (20 mL) and extracted with ethyl acetate (5 mL×4). The combined organic layers were washed with brine (10 mL), dried over anhydrous sodium sulfate, filtered and the filtrate was concentrated under reduced pressure. The residue was purified by column chromatography to afford the title compound. ¹H NMR (400 MHz, CD₃OD) δ=7.41 (d, J=2.4 Hz, 1H), 7.09-7.06 (m, 1H), 5.39 (d, J=6.0 Hz, 1H), 5.19 (d, J=5.6 Hz, 1H), 4.53 (d, J=6.0 Hz, 1H), 4.32 (d, J=5.6 Hz, 1H), 4.17-4.07 (m, 2H), 3.89 (t, J=8.4 Hz, 1H), 3.40 (t, J=9.6 Hz, 1H), 3.30-3.24 (m, 1H), 3.08-3.01 (m, 1H), 2.86-2.74 (m, 2H), 2.68-2.61 (m, 1H), 2.57-2.49 (m, 1H), 1.89-1.79 (m, 2H), 1.43 (s, 8H). LCMS R_{T}=2.553 min, m/z=478.8 [M+H]⁺.

### Step 8: 7-(8-(2-(((tert-butyldimethylsilyl)oxy)methyl)thieno[3,2-b]pyridin-7-yl)-6-chloro-3,4-dihydroquinolin-1(2H)-yl)-5-(tert-butylsulfonyl)-2-oxa-5-azaspiro[3.4]octane

To a solution of 7-(8-bromo-6-chloro-3,4-dihydroquinolin-1(2H)-yl)-5-(tert-butylsulfonyl)-2-oxa-5-azaspiro[3.4]octane (200 mg, 0.42 mmol) in water (0.2 mL) and dioxane (1 mL) was added (2-(((tert-butyldimethylsilyl)oxy)methyl)thieno[3,2-b]pyridin-7-yl)boronic acid (271 mg, 0.84 mmol), dichloro[1,1′-bis(diphenylphosphino)ferrocene]palladium(II) (61 mg, 0.084 mmol) and potassium carbonate (174 mg, 1.26 mmol) at 20° C. After stirred at 100° C. for 2 h under nitrogen atmosphere, the mixture was filtered and the filtrate was concentrated under reduced pressure to remove the solvent. The residue was purified by preparative TLC to afford the title compound. LCMS R_{T}=0.645 min, m/z=676.3 [M+H]⁺.

### Step 9: (S)-(7-(1-(5-(tert-butylsulfonyl)-2-oxa-5-azaspiro[3.4]octan-7-yl)-6-chloro-1,2,3,4-tetrahydroquinolin-8-yl)thieno[3,2-b]pyridin-2-yl)methanol and (R)-(7-(1-(5-(tert-butylsulfonyl)-2-oxa-5-azaspiro[3.4]octan-7-yl)-6-chloro-1,2,3,4-tetrahydroquinolin-8-yl)thieno[3,2-b]pyridin-2-yl)methanol

To a solution of 7-(8-(2-(((tert-butyldimethylsilyl)oxy)methyl)thieno[3,2-b]pyridin-7-yl)-6-chloro-3,4-dihydroquinolin-1(2H)-yl)-5-(tert-butylsulfonyl)-2-oxa-5-azaspiro[3.4]octane (250 mg, 0.37 mmol) in THF (1 mL) was added tetrabutylammonium fluoride (1 mL, 1 M in THF). After stirred at 20° C. for 0.5 h, the mixture was concentrated under reduced pressure. The residue was purified by column chromatography. The racemic product was separated by SFC to afford first eluting fraction (181.1, Rₜ=3.803 min; LCMS R_{T}=0.871 min, m/z=562.0 [M+H]+) and second eluting fraction (181.1, R₁=4.166 min; LCMS R_{T}=0.871 min, m/z=562.0 [M+H]+).

### Step 10

Example 181a was prepared from 181.1 and succinimide, following the procedure described in the synthesis of Example 108. The product was purified by preparative HPLC. ¹H NMR (400 MHz, CD₃OD) δ=8.96 (d, J=6.0 Hz, 1H), 8.02 (d, J=5.6 Hz, 1H), 7.79 (s, 1H), 7.31 (dd, J=2.4, 11.2 Hz, 2H), 5.07 (s, 2H), 3.70-3.69 (m, 1H), 3.58-3.42 (m, 2H), 3.41-3.32 (m, 1H), 3.31-3.04 (m, 1H), 3.31-3.04 (m, 3H), 2.92 (t, J=6.4 Hz, 2H), 2.79 (s, 4H), 2.23-0.31 (m, 5H). LCMS R_{T}=1.080 min, m/z=541.4 [M+H]⁺.

Example 181b was prepared from 181.2 and succinimide, following the procedure described in the synthesis of Example 108. The product was purified by preparative HPLC. ¹H NMR (400 MHz, CD₃OD) δ=8.96 (d, J=6.0 Hz, 1H), 8.02 (d, J=5.6 Hz, 1H), 7.79 (s, 1H), 7.31 (dd, J=2.4, 11.2 Hz, 2H), 5.07 (s, 2H), 3.70-3.69 (m, 1H), 3.58-3.42 (m, 2H), 3.41-3.32 (m, 1H), 3.31-3.04 (m, 1H), 3.31-3.04 (m, 3H), 2.92 (t, J=6.4 Hz, 2H), 2.79 (s, 4H), 2.23-0.31 (m, 5H). LCMS R_{T}=0.462, m/z=541.1 [M+H]⁺.

### Example 182: 1-((7-(6-chloro-1-((3R,5S)-5-(hydroxymethyl)pyrrolidin-3-yl)-1,2,3,4-tetrahydroquinolin-8-yl)thieno[3,2-b]pyridin-2-yl)methyl)pyrrolidine-2,5-dione, formic acid salt

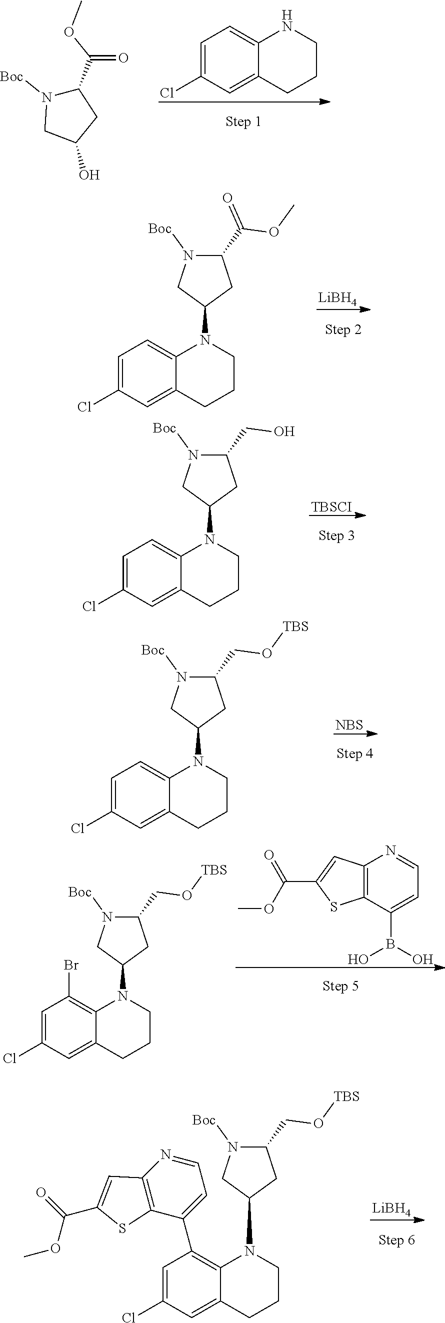

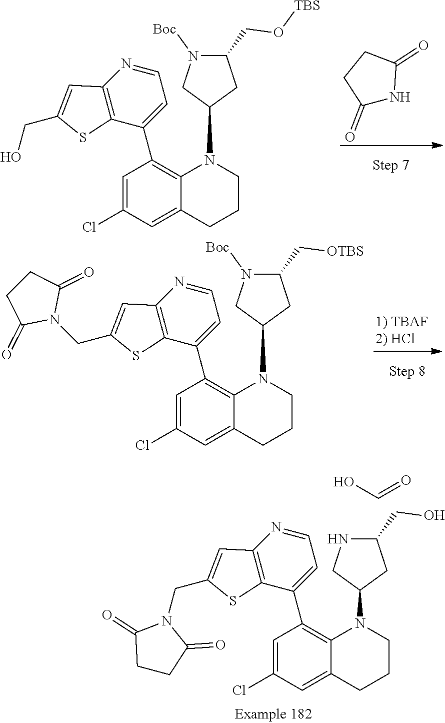

### Step 1: (2S,4R)-1-tert-butyl 2-methyl 4-(6-chloro-3,4-dihydroquinolin-1(2H)-yl)pyrrolidine-1,2-dicarboxylate

To a solution of 6-chloro-1,2,3,4-tetrahydroquinoline (5.4 g, 32.62 mmol) in methylene chloride (25 mL) was added N, N-diisopropylethylamine (3.9 g, 30.58 mmol) and trifluoromethylanhydride (6.9 g, 24.46 mmol) at −65° C. The mixture was allowed to warm to −30° C. and added a solution of (2S,4S)-1-tert-butyl 2-methyl 4-hydroxypyrrolidine-1,2-dicarboxylate (5.0 g, 20.39 mmol) in methylene chloride (25 mL). After stirred at 25° C. for 3 h, the reaction mixture was quenched with saturated aqueous sodium bicarbonate (50 mL), extracted with methylene chloride (20 mL×4) and water (10 mL×4), and dried over anhydrous sodium sulfate, filtered and the filtrate was concentrated under reduced pressure. The residue was purified by column chromatography to afford the title compound. LCMS R_{T}=0.620, m/z=395.2 [M+H]⁺.

### Step 2: (2S,4R)-tert-butyl 4-(6-chloro-3,4-dihydroquinolin-1(2H)-yl)-2-(hydroxymethyl)pyrrolidine-1-carboxylate

To a solution of (2S,4R)-1-tert-butyl 2-methyl 4-(6-chloro-3,4-dihydroquinolin-1(2H)-yl)pyrrolidine-1,2-dicarboxylate (6.7 g, 16.97 mmol) in THF (50 mL) was added lithium borohydride (740 mg, 33.93 mmol) at −78° C. After stirred at 25° C. for 12 h, the reaction mixture was quenched with saturated aqueous ammonium chloride (50 mL), the residue was extracted with ethyl acetate (25 mL×4). The organic layers were washed with water (10 mL×4), dried over anhydrous sodium sulfate, filtered and the filtrate was concentrated under reduced pressure. The residue was purified by column chromatography to afford the title compound. LCMS R_{T}=1.027 min, m/z=367.1 [M+H]⁺.

### Step 3: (2S,4R)-tert-butyl 2-(((tert-butyldimethylsilyl)oxy)methyl)-4-(6-chloro-3,4-dihydroquinolin-1(2H)-yl)pyrrolidine-1-carboxylate

To a solution of (2S,4R)-tert-butyl 4-(6-chloro-3,4-dihydroquinolin-1(2H)-yl)-2-(hydroxymethyl)pyrrolidine-1-carboxylate (6.9 g, 18.81 mmol) in methylene chloride (50 mL) was added tert-butyldimethylsilyl chloride (3.4 g, 22.57 mmol) and imidazole (3.8 g, 56.42 mmol) at 25° C. After stirred at 25° C. for 2 h, the reaction mixture was quenched with water (80 mL). The mixture was extracted with methylene chloride (10 mL×4). The combined organic layers were washed with water (10 mL×4), dried over anhydrous sodium sulfate, filtered and the filtrate was concentrated under reduced pressure. The residue was purified by column chromatography to afford the title compound. LCMS R_{T}=0.757 min, m/z=481.3 [M+H]⁺.

### Step 4: (2S,4R)-tert-butyl 4-(8-bromo-6-chloro-3,4-dihydroquinolin-1(2H)-yl)-2-(((tert-butyldimethylsilyl)oxy)methyl)pyrrolidine-1-carboxylate

To a solution of (2S,4R)-tert-butyl 2-(((tert-butyldimethylsilyl)oxy)methyl)-4-(6-chloro-3,4-dihydroquinolin-1(2H)-yl)pyrrolidine-1-carboxylate (7.2 g, 14.96 mmol) in methylene chloride (70 mL) was added 1-bromopyrrolidine-2,5-dione (3.5 g, 19.45 mmol) at 0° C. After stirred at 25° C. for 0.5 h, the reaction mixture was concentrated under reduced pressure. The residue was purified by column chromatography to afford the title compound. LCMS R_{T}=3.483 min, m/z=561.2 [M+H]⁺.

### Step 5: methyl 7-(1-((3R,5S)-1-(tert-butoxycarbonyl)-5-(((tert-butyldimethylsilyl)oxy)methyl)pyrrolidin-3-yl)-6-chloro-1,2,3,4-tetrahydroquinolin-8-yl)thieno[3,2-b]pyridine-2-carboxylate

To a solution of (2S,4R)-tert-butyl 4-(8-bromo-6-chloro-3,4-dihydroquinolin-1(2H)-yl)-2-(((tert-butyldimethylsilyl)oxy)methyl)pyrrolidine-1-carboxylate (400 mg, 0.7 mmol) in dioxane (2 mL) and water (0.1 mL) was added (2-methoxycarbonylthieno[3,2-b]pyridin-7-yl) boronic acid (340 mg, 1.43 mmol), palladium dichloride[1,1′-bis (diphenylphosphine) ferrocene] (100 mg, 0.1 mmol) and cesium carbonate (470 mg, 1.43 mmol) at 25° C. After stirred at 100° C. for 3 h, the reaction mixture was concentrated under reduced pressure. The residue was purified by column chromatography to afford the title compound. LCMS R_{T}=0.737 min, m/z=672.4 [M+H]⁺.

### Step 6: (2S,4R)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)pyrrolidine-1-carboxylate

To a solution of methyl 7-(1-((3R,5S)-1-(tert-butoxycarbonyl)-5-(((tert-butyldimethylsilyl)oxy)methyl)pyrrolidin-3-yl)-6-chloro-1,2,3,4-tetrahydroquinolin-8-yl)thieno[3,2-b]pyridine-2-carboxylate (550 mg, 0.81 mmol) in THF (5 mL) was added lithium borohydride (4 mg, 0.2 mmol) at −78° C. After stirred at 25° C. for 12 h, the reaction mixture was quenched with saturated aqueous ammonium chloride (10 mL). The mixture was extracted by ethyl acetate (5 mL×4). The combined organic layers were washed with water (10 mL×4), dried over anhydrous sodium sulfate, filtered and the filtrate was concentrated under reduced pressure. The residue was purified by column chromatography to afford the title compound. LCMS R_{T}=0.690 min, m/z=644.4 [M+H]⁺.

### Step 7: (2S,4R)-tert-butyl 2-(((tert-butyldimethylsilyl)oxy)methyl)-4-(6-chloro-8-(2-((2,5-dioxopyrrolidin-1-yl)methyl)thieno[3,2-b]pyridin-7-yl)-3,4-dihydroquinolin-1(2H)-yl)pyrrolidine-1-carboxylate

To a solution of (2S,4R)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)pyrrolidine-1-carboxylate (150 mg, 0.2 mmol) in toluene (0.5 mL) was added succinimide (50 mg, 0.5 mmol), tetramethylazodicarboxamide (160 mg, 0.9 mmol) and tributylphosphane (280 mg, 1.40 mmol) at 25° C. After stirred at 100° C. for 1 h, the reaction mixture was concentrated under reduced pressure. The residue was purified by preparative TLC to afford the title compound. LCMS R_{T}=0.643, m/z=725.5 [M+H]⁺.

### Step 8

To a solution of (2S,4R)-tert-butyl 2-(((tert-butyldimethylsilyl)oxy)methyl)-4-(6-chloro-8-(2-((2,5-dioxopyrrolidin-1-yl)methyl)thieno[3,2-b]pyridin-7-yl)-3,4-dihydroquinolin-1(2H)-yl)pyrrolidine-1-carboxylate (30 mg, 0.04 mmol) in methylene chloride (1 mL) was added trifluoroacetic acid (5 mg, 0.04 mmol) at 25° C. After stirred at 25° C. for 1.5 h, the mixture was added hydrochloric acid (0.02 mL, 4 M in dioxane) and stirred for 0.5 h. The reaction mixture was concentrated under reduced pressure. The residue was purified by preparative HPLC to afford Example 182. ¹H NMR (400 MHz, CD₃OD) δ=8.66-8.73 (m, 1H), 8.49 (s, 1H), 7.41-7.50 (m, 2H), 7.15 (d, J=10.0 Hz, 2H), 4.94 (s, 2H), 3.36-3.83 (m, 4H), 3.24 (s, 2H), 2.87 (s, 3H), 2.75 (s, 5H), 1.73-2.05 (m, 4H). LCMS R_{T}=1.130 min, m/z=511.4 [M+H]⁺.

### Example 183: 1-[[7-[6-chloro-1-[(3S,5R)-5-(hydroxymethyl)pyrrolidin-3-yl]-3,4-dihydro-2H-quinolin-8-yl]thieno[3,2-b]pyridin-2-yl]methyl]pyrrolidine-2,5-dione, formic acid salt

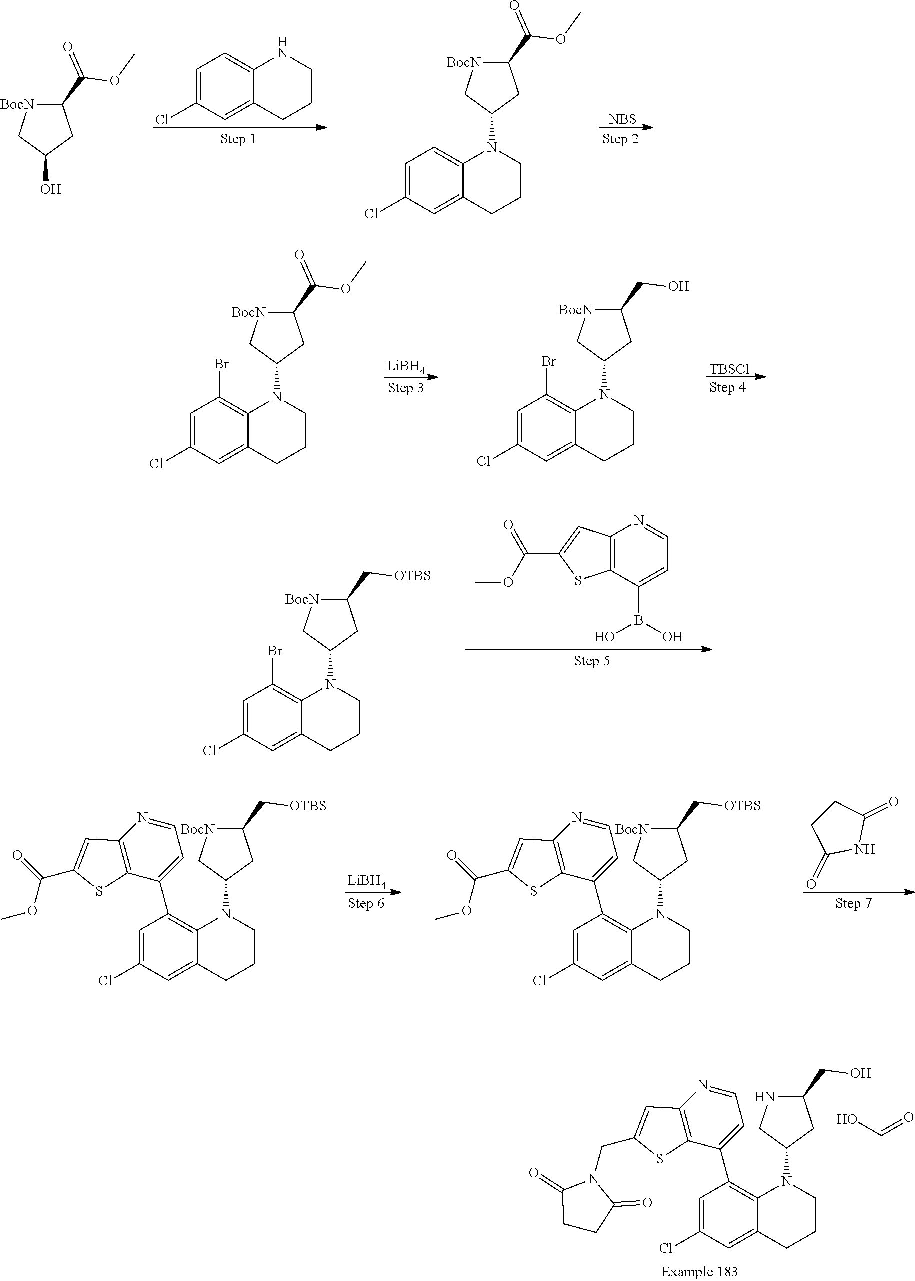

### Step 1: (2R,4S)-1-tert-butyl 2-methyl 4-(6-chloro-3,4-dihydroquinolin-1(2H)-yl)pyrrolidine-1,2-dicarboxylate

To a solution of (2R,4R)-1-tert-butyl 2-methyl 4-hydroxypyrrolidine-1,2-dicarboxylate (3.0 g, 12.23 mmol) in dichloromethane (30 mL) was added diisopropyl ethyl amine (2.4 g, 18.35 mmol) and trifluoromethylanhydride (4.2 g, 14.68 mmol) and 6-chloro-1,2,3,4-tetrahydroquinoline (3.3 g, 19.57 mmol) at −65° C. After stirred at 25° C. for 12 h, the reaction mixture was quenched by addition of saturated aqueous sodium bicarbonate (100 mL), and then extracted with dichloromethane (50 mL×3). The combined organic layers were dried over sodium sulphate, filtered and the filtrate was concentrated under reduced pressure to give a residue. The residue was purified by column chromatography to afford the title compound. LCMS R_{T}=2.283 min, m/z=395.3 [M+H]⁺.

### Step 2: (2R,4S)-1-tert-butyl 2-methyl 4-(8-bromo-6-chloro-3,4-dihydroquinolin-1(2H)-yl)pyrrolidine-1,2-dicarboxylate

To a solution of (2R,4S)-1-tert-butyl 2-methyl 4-(6-chloro-3,4-dihydroquinolin-1(2H)-yl)pyrrolidine-1,2-dicarboxylate (700 mg, 1.42 mmol) in dichloromethane (10 mL) was added N-bromosuccinimide (379 mg, 2.13 mmol) at 0° C. After stirred at 25° C. for 3 h, the reaction mixture was quenched by addition of saturated aqueous ammonium chloride (30 mL), and then extracted with dichloromethane (10 mL×3). The combined organic layers were dried over sodium sulphate, filtered and the filtrate was concentrated under reduced pressure to give a residue. The residue was purified by column chromatography to afford the title compound. LCMS R_{T}=1.137 min, m/z=474.9 [M+H]⁺.

### Step 3: (2R,4S)-tert-butyl 4-(8-bromo-6-chloro-3,4-dihydroquinolin-1(2H)-yl)-2-(hydroxymethyl)pyrrolidine-1-carboxylate

To a solution of (2R,4S)-1-tert-butyl 2-methyl 4-(8-bromo-6-chloro-3,4-dihydroquinolin-1(2H)-yl)pyrrolidine-1,2-dicarboxylate (700 mg, 1.48 mmol) in THF (10 mL) was added lithium borohydride (97 mg, 4.43 mmol) at 0° C. After stirred at 25° C. for 12 h, the reaction mixture was quenched by addition of saturated aqueous ammonium chloride (20 mL), and then extracted with ethyl acetate (20 mL×3). The combined organic layers were dried over sodium sulphate, filtered and the filtrate was concentrated under reduced pressure to give a residue. The residue was purified by column chromatography to afford the title compound. LCMS R_{T}=1.587 min, m/z=447.2 [M+2+H]⁺.

### Step 4: (2R,4S)-tert-butyl 4-(8-bromo-6-chloro-3,4-dihydroquinolin-1(2H)-yl)-2-(((tert-butyldimethylsilyl)oxy)methyl)pyrrolidine-1-carboxylate

To a solution of (2R,4S)-tert-butyl 4-(8-bromo-6-chloro-3,4-dihydroquinolin-1(2H)-yl)-2-(hydroxymethyl)pyrrolidine-1-carboxylate (560 mg, 1.26 mmol) in dichloromethane (10 mL) was added imidazole (342 mg, 5.02 mmol) and tert-butyldimethylsilyl chloride (474 mg, 3.14 mmol). After stirred at 25° C. for 12 h, the reaction mixture was quenched by addition of saturated aqueous ammonium chloride (20 mL), and then extracted with dichloromethane (10 mL×3). The combined organic layers were dried over sodium sulphate, filtered and the filtrate was concentrated under reduced pressure to give a residue. The residue was purified by column chromatography to afford the title compound. LCMS R_{T}=0.842 min, m/z=560.9 [M+H]⁺.

### Step 5: methyl 7-(1-((3S,5R)-1-(tert-butoxycarbonyl)-5-(((tert-butyldimethylsilyl)oxy)methyl)pyrrolidin-3-yl)-6-chloro-1,2,3,4-tetrahydroquinolin-8-yl)thieno[3,2-b]pyridine-2-carboxylate

To a solution of (2R,4S)-tert-butyl 4-(8-bromo-6-chloro-3,4-dihydroquinolin-1(2H)-yl)-2-(((tert-butyldimethylsilyl)oxy)methyl)pyrrolidine-1-carboxylate (550 mg, 0.98 mmol) in dioxane (8 mL) and water (2 mL) was added (2-methoxycarbonylthieno[3,2-b]pyridin-7-yl)boronic acid (349 mg, 1.47 mmol) and dichloro[1,1′-bis(diphenylphosphino)ferrocene]palladium(II) (144 mg, 0.20 mmol) and cesium carbonate (640 mg, 1.96 mmol) under nitrogen atmosphere. After stirred at 120° C. for 1 h, the reaction mixture was diluted with water (10 mL), extracted with ethyl acetate (10 mL×2). The combined organic layers were washed with brine (10 mL), dried over anhydrous sodium sulfate and concentrated under reduced pressure. The residue was purified by column chromatography to afford the title compound. LCMS R_{T}=2.387 min, m/z=672.3 [M+H]⁺.

### Step 6: (2R,4S)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)pyrrolidine-1-carboxylate

To a solution of methyl 7-(1-((3S,5R)-1-(tert-butoxycarbonyl)-5-(((tert-butyldimethylsilyl)oxy)methyl)pyrrolidin-3-yl)-6-chloro-1,2,3,4-tetrahydroquinolin-8-yl)thieno[3,2-b]pyridine-2-carboxylate (300 mg, 0.45 mmol) in THF (5 mL) was added lithium borohydride (58 mg, 2.68 mmol) at 0° C. After stirred at 25° C. for 12 h, the reaction mixture was quenched by addition of saturated aqueous ammonium chloride (10 mL), and then extracted with ethyl acetate (10 mL×3). The combined organic layers were dried over sodium sulphate, filtered and the filtrate was concentrated under reduced pressure to give a residue. The residue was purified by column chromatography to afford the title compound. LCMS R_{T}=0.689 min, m/z=644.4 [M+H]⁺.

### Step 7

Example 183 was prepared from (2R,4S)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)pyrrolidine-1-carboxylate in toluene (5 mL) and succinimide, following the procedure described in the synthesis of Example 108. The product was purified by preparative HPLC. To a solution of ¹H NMR (400 MHz, CD₃OD) δ=8.70 (d, J=4.8 Hz, 1H), 8.42 (s, 1H), 7.49-7.41 (m, 2H), 7.15 (dd, J=2.4, 11.6 Hz, 2H), 4.94 (s, 2H), 3.83-3.36 (m, 3H), 3.24 (s, 2H), 3.14-2.83 (m, 4H), 3.14-2.83 (m, 5H), 2.17-1.73 (m, 4H). LCMS R_{T}=0.449 min, m/z=511.2 [M+H]⁺.

### Example 184: 1-((7-(6-chloro-1-((3S,5S)-5-(hydroxymethyl)pyrrolidin-3-yl)-1,2,3,4-tetrahydroquinolin-8-yl)thieno[3,2-b]pyridin-2-yl)methyl)pyrrolidine-2,5-dione, formic acid salt

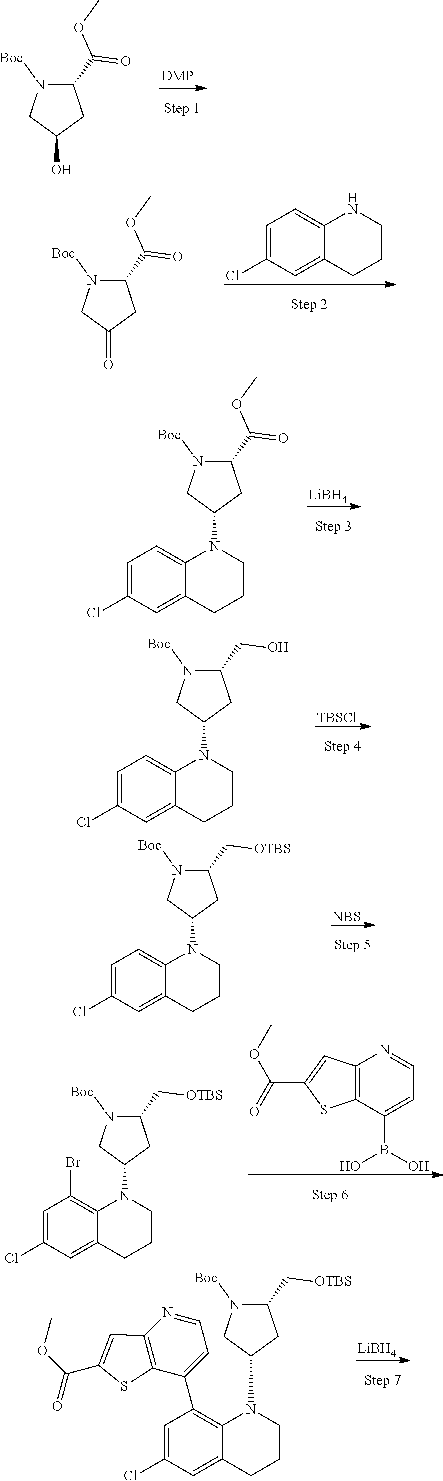

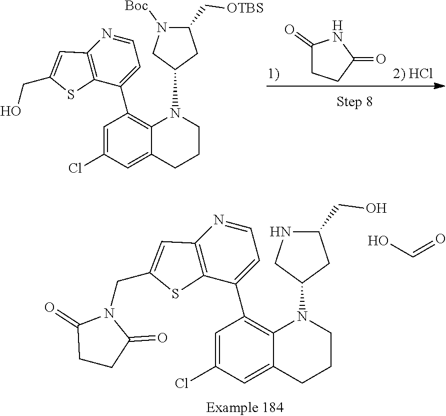

### Step 1: (S)-1-tert-butyl 2-methyl 4-oxopyrrolidine-1,2-dicarboxylate

To a solution of (2S,4R)-1-tert-butyl 2-methyl 4-hydroxypyrrolidine-1,2-dicarboxylate (5 g, 20.39 mmol) in dichloromethane (200 mL) was added dess-martin periodinane (10.38 g, 24.46 mmol). After stirred at 20° C. for 3 h, the mixture was quenched by addition of water (150 mL) and the mixture was filtered. The organic layer was separated and washed with brine (100 mL), dried over anhydrous sodium sulfate and concentrated under reduced pressure. The residue was purified by column chromatography to afford the title compound. ¹H NMR (400 MHz, CDCl₃) δ=4.87-4.65 (m, 1H), 3.95-3.85 (m, 2H), 3.79-3.74 (m, 3H), 2.94 (dt, J=10.8, 17.6 Hz, 1H), 2.58 (d, J=18.8 Hz, 1H), 1.47 (d, J=8.8 Hz, 9H).

### Step 2: (2S,4S)-1-tert-butyl 2-methyl 4-(6-chloro-3,4-dihydroquinolin-1(2H)-yl)pyrrolidine-1,2-dicarboxylate

To a solution of (S)-1-tert-butyl 2-methyl 4-oxopyrrolidine-1,2-dicarboxylate (2.9 g, 11.93 mmol) and 6-chloro-1,2,3,4-tetrahydroquinoline (2 g, 11.93 mmol) in acetonitrile (60 mL) was added trifluoroacetic acid (6.12 g, 53.69 mmol) and sodium borohydride acetate (6.32 g, 29.83 mmol). After stirred at 35° C. for 3 h, the mixture was quenched by addition of saturated aqueous sodium hydrogencarbonate (70 mL) and concentrated to remove acetonitrile. The mixture was extracted with ethyl acetate (80 mL). The organic layer was washed with brine (50 mL), dried over anhydrous sodium sulfate and concentrated under reduced pressure. The residue was purified by column chromatography to afford the title compound. LCMS R_{T}=1.103 min, m/z=395.0 [M+H]⁺.

### Step 3: (2S,4S)-tert-butyl 4-(6-chloro-3,4-dihydroquinolin-1(2H)-yl)-2-(hydroxymethyl)pyrrolidine-1-carboxylate

To a solution of (2S,4S)-1-tert-butyl 2-methyl 4-(6-chloro-3,4-dihydroquinolin-1(2H)-yl)pyrrolidine-1,2-dicarboxylate (3.4 g, 8.61 mmol) in THF (50 mL) was added lithium borohydride (0.54 g, 24.79 mmol) at −65° C. After stirred at 15° C. for 15 h, the mixture was quenched by addition of saturated aqueous ammonium chloride (80 mL) and extracted with ethyl acetate (60 mL×2). The combined organic layers were washed with brine (80 mL), dried over anhydrous sodium sulfate and concentrated under reduced pressure. The residue was purified by column chromatography to afford the title compound. LCMS R_{T}=1.703 min, m/z=366.9 [M+H]⁺.

### Step 4: (2S,4S)-tert-butyl 2-(((tert-butyldimethylsilyl)oxy)methyl)-4-(6-chloro-3,4-dihydroquinolin-1(2H)-yl)pyrrolidine-1-carboxylate

To a solution of (2S,4S)-tert-butyl 4-(6-chloro-3,4-dihydroquinolin-1(2H)-yl)-2-(hydroxymethyl)pyrrolidine-1-carboxylate (3.1 g, 8.45 mmol) in dichloromethane (50 mL) was added 1H-imidazole (1.44 g, 21.12 mmol) and tert-butylchlorodimethylsilane (1.78 g, 11.83 mmol). After stirred at 15° C. for 2 h, the mixture was diluted with water (60 mL) and extracted with dichloromethane (50 mL). The organic layer was washed with brine (50 mL), dried over anhydrous sodium sulfate and concentrated under reduced pressure. The residue was purified by column chromatography to afford the title compound. LCMS R_{T}=2.760 min, m/z=481.5 [M+H]⁺.

### Step 5: (2S,4S)-tert-butyl 4-(8-bromo-6-chloro-3,4-dihydroquinolin-1(2H)-yl)-2-(((tert-butyldimethylsilyl)oxy)methyl)pyrrolidine-1-carboxylate

To a solution of (2S,4S)-tert-butyl 2-(((tert-butyldimethylsilyl)oxy)methyl)-4-(6-chloro-3,4-dihydroquinolin-1(2H)-yl)pyrrolidine-1-carboxylate (600 mg, 1.25 mmol) in DMF (8 mL) was added 1-bromopyrrolidine-2,5-dione (222 mg, 1.25 mmol) at 0° C. After stirred at 0° C. for 1 h, the mixture was diluted with ethyl acetate (50 mL) and washed with water (50 mL×2). The organic layer was dried over anhydrous sodium sulfate and concentrated under reduced pressure. The residue was purified by column chromatography to afford the title compound. LCMS R_{T}=1.406 min, m/z=599.0 [M+H]⁺.

### Step 6: methyl 7-(1-((3S,5S)-1-(tert-butoxycarbonyl)-5-(((tert-butyldimethylsilyl)oxy)methyl)pyrrolidin-3-yl)-6-chloro-1,2,3,4-tetrahydroquinolin-8-yl)thieno[3,2-b]pyridine-2-carboxylate

To a solution of (2S,4S)-tert-butyl 4-(8-bromo-6-chloro-3,4-dihydroquinolin-1(2H)-yl)-2-(((tert-butyldimethylsilyl)oxy)methyl)pyrrolidine-1-carboxylate (250 mg, 0.45 mmol) and (2-(methoxycarbonyl)thieno[3,2-b]pyridin-7-yl)boronic acid (138 mg, 0.58 mmol) in water (0.5 mL) and dioxane (6 mL) was added dichloro[1,1′-bis(diphenylphosphino)ferrocene]palladium(II) (33 mg, 0.045 mmol) and potassium carbonate (123 mg, 0.89 mmol). After stirred at 100° C. for 2 h under nitrogen atmosphere, the mixture was concentrated under reduced pressure. The residue was purified by column chromatography to afford the title compound. LCMS R_{T}=2.413 min, m/z=672.4 [M+H]⁺.

### Step 7: (2S,4S)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)pyrrolidine-1-carboxylate

To a solution of methyl 7-(1-((3S,5S)-1-(tert-butoxycarbonyl)-5-(((tert-butyldimethylsilyl)oxy)methyl)pyrrolidin-3-yl)-6-chloro-1,2,3,4-tetrahydroquinolin-8-yl)thieno[3,2-b]pyridine-2-carboxylate (200 mg, 0.30 mmol) in THF (4 mL) was added lithium borohydride (19 mg, 0.90 mmol) at 0° C. After stirred at 20° C. for 15 h, the mixture was quenched by addition of saturated aqueous ammonium chloride (20 mL) and extracted with ethyl acetate (15 mL). The organic layer was washed with brine (15 mL), dried over anhydrous sodium sulfate and concentrated under reduced pressure. The residue was purified by preparative TLC to afford the title compound. LCMS R_{T}=2.167 min, m/z=644.4 [M+H]⁺.

### Step 8

Example 184 was prepared from (2S,4S)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)pyrrolidine-1-carboxylate and succinimide, following the procedure described in the synthesis of Example 108. ¹H NMR (400 MHz, CD₃OD) δ=8.72 (d, J=4.8 Hz, 1H), 8.50 (s, 1H), 7.51-7.45 (m, 2H), 7.17 (dd, J=2.4, 11.6 Hz, 2H), 4.94 (s, 2H), 3.74-3.59 (m, 2H), 3.53-3.43 (m, 1H), 3.31-3.04 (m, 5H), 2.89 (t, J=6.4 Hz, 2H), 2.75 (s, 4H), 2.09-1.64 (m, 4H). LCMS R_{T}=1.073 min, m/z=511.4 [M+H]⁺.

### Example 185: 3-((7-(6-chloro-1-((3S,5R)-5-(hydroxymethyl)pyrrolidin-3-yl)-1,2,3,4-tetrahydroquinolin-8-yl)thieno[3,2-b]pyridin-2-yl)methyl)-3-azabicyclo[3.1.0]hexane-2,4-dione, formic acid salt

Example 185 was prepared from tert-butyl (2R,4S)-2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)pyrrolidine-1-carboxylate and 3-azabicyclo[3.1.0]hexane-2,4-dione, following the procedure described in the synthesis of Example 108. ¹H NMR (400 MHz, CD₃OD) δ=8.67-8.71 (m, 1H), 8.51-8.54 (m, 1H), 7.39-7.45 (m, 2H), 7.12-7.18 (m, 2H), 4.78-4.80 (m, 2H), 3.64-3.74 (m, 1H), 3.33-3.50 (m, 4H), 3.23 (s, 2H), 2.98-3.14 (m, 1H), 2.86 (t, J=6.16 Hz, 2H), 2.58 (dd, J=8.12, 3.64 Hz, 2H), 1.70-2.19 (m, 4H), 1.61 (td, J=8.07, 4.50 Hz, 1H), 1.39 (q, J=3.68 Hz, 1H). LCMS R_{T}=1.137 min, m/z=523.3 [M+H]⁺.

### Example 186: 2-[[7-[6-chloro-1-[(3S,5R)-5-(hydroxymethyl)pyrrolidin-3-yl]-3,4-dihydro-2H-quinolin-8-yl]thieno[3,2-b]pyridin-2-yl]methyl]-5-cyclopropyl-pyridazin-3-one, formic acid salt

Example 186 was prepared from tert-butyl (2R,4S)-2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)pyrrolidine-1-carboxylate and 4-cyclopropyl-1H-pyridazin-6-one, following the procedure described in the synthesis of Example 108. ¹H NMR (400 MHz, CD₃OD) δ=8.69 (d, J=4.8 Hz, 1H), 7.79 (d, J=2.4 Hz, 1H), 7.51 (s, 1H), 7.45-7.11 (m, 3H), 6.60 (d, J=2.4 Hz, 1H), 5.58 (s, 2H), 3.82-3.33 (m, 3H), 3.18 (s, 2H), 3.12-2.70 (m, 5H), 2.04-1.75 (m, 4H), 1.21-1.11 (m, 2H), 0.94-0.86 (m, 2H), 0.82-0.24 (m, 1H). LCMS R_{T}=1.263 min, m/z=548.4 [M+H]⁺.

### Example 187: 2-((7-(6-chloro-1-((3S,5S)-5-(hydroxymethyl)pyrrolidin-3-yl)-1,2,3,4-tetrahydroquinolin-8-yl)thieno[3,2-b]pyridin-2-yl)methyl)-5-cyclopropylpyridazin-3(2H)-one, formic acid salt

Example 187 was prepared from tert-butyl (2S,4S)-2-[[tert-butyl(dimethyl)silyl]oxymethyl]-4-[6-chloro-8-[2-(hydroxymethyl)thieno[3,2-b]pyridin-7-yl]-3,4-dihydro-2H-quinolin-1-yl]pyrrolidine-1-carboxylate (60 mg, 0.09 mmol) in toluene (1.5 mL) and 4-cyclopropyl-1H-pyridazin-6-one (38 mg, 0.28 mmol), following the procedure described in the synthesis of Example 108. The product was purified by preparative HPLC. ¹H NMR (400 MHz, CD₃OD) δ=8.75-8.64 (m, 1H), 8.54-8.40 (m, 1H), 7.81-7.74 (m, 1H), 7.53-7.42 (m, 2H), 7.20-7.07 (m, 2H), 6.68-6.46 (m, 1H), 5.72-0.37 (m, 2H), 3.78-3.36 (m, 5H), 3.27-3.02 (m, 3H), 2.96-2.74 (m, 2H), 2.21-1.48 (m, 5H), 1.22-1.08 (m, 2H), 0.94-0.81 (m, 2H). LCMS R_{T}=1.384 min, m/z=548.1 [M+H]⁺.

### Example 188: 2-((7-(6-chloro-1-((3S,5R)-5-(hydroxymethyl)pyrrolidin-3-yl)-1,2,3,4-tetrahydroquinolin-8-yl)thieno[3,2-b]pyridin-2-yl)methyl)-5-(1-methylcyclopropyl)pyridazin-3(2H)-one, formic acid salt

Example 188 was prepared from (2R,4S)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)pyrrolidine-1-carboxylate and 4-(1-methylcyclopropyl)-1H-pyridazin-6-one, following the procedure described in the synthesis of Example 108. The product was purified by preparative HPLC. ¹H NMR (400 MHz, CD₃OD) δ=8.72-8.67 (m, 1H), 8.54-8.49 (m, 1H), 7.76-7.72 (m, 1H), 7.52 (s, 1H), 7.46-7.41 (m, 1H), 7.18-7.12 (m, 2H), 6.82-6.72 (m, 1H), 5.62-5.54 (m, 2H), 3.84-3.81 (m, 1H), 3.83-3.64 (m, 1H), 3.32 (s, 3H), 3.24-3.15 (m, 2H), 3.04 (s, 4H), 2.07-1.80 (m, 3H), 1.39 (s, 3H), 1.14-1.08 (m, 2H), 1.01-0.95 (m, 2H). LCMS R_{T}=1.380 min, m/z=562.4 [M+H]⁺.

### Example 189: 1-((7-(6-chloro-1-((3S,5R)-5-(2-hydroxypropan-2-yl)pyrrolidin-3-yl)-1,2,3,4-tetrahydroquinolin-8-yl)thieno[3,2-b]pyridin-2-yl)methyl)pyrrolidine-2,5-dione, formic acid salt

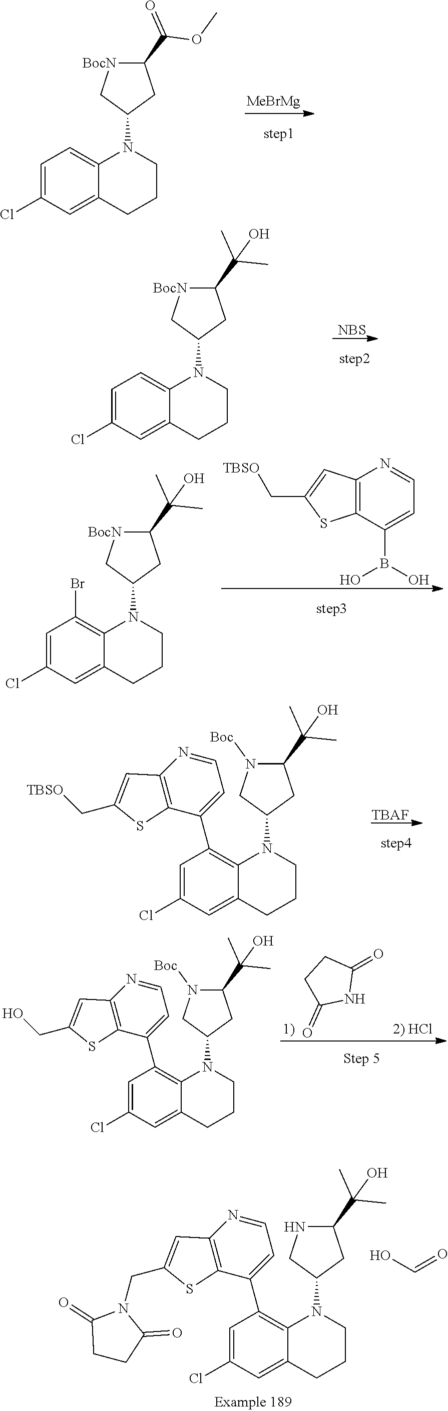

### Step 1: (2R,4S)-tert-butyl 4-(6-chloro-3,4-dihydroquinolin-1(2H)-yl)-2-(2-hydroxypropan-2-yl)pyrrolidine-1-carboxylate

To a solution of (2R,4S)-1-tert-butyl 2-methyl 4-(6-chloro-3,4-dihydroquinolin-1(2H)-yl)pyrrolidine-1,2-dicarboxylate (1.2 g, 3.04 mmol) in THF (2 mL) was added bromo(methyl)magnesium (3 M in THF, 5.06 mL) at 0° C. After stirred at 0° C. for 1 h, the reaction mixture was quenched by addition of saturated aqueous ammonium chloride solution (50 mL) and extracted with ethyl acetate (20 mL×2). The combined organic layers were washed with brine (20 mL), dried over anhydrous sodium sulfate, filtered and the filtrate was concentrated under reduced pressure. The residue was purified by column chromatography to afford the title compound. LCMS R_{T}=0.619 min, m/z=395.2 [M+H]⁺

### Step 2: (2R,4S)-tert-butyl 4-(8-bromo-6-chloro-3,4-dihydroquinolin-1(2H)-yl)-2-(2-hydroxypropan-2-yl)pyrrolidine-1-carboxylate

To a solution of (2R,4S)-tert-butyl 4-(6-chloro-3,4-dihydroquinolin-1(2H)-yl)-2-(2-hydroxypropan-2-yl)pyrrolidine-1-carboxylate (500 mg, 1.27 mmol) in dichloromethane (10 mL) was added N-bromosuccinimide (225 mg, 1.27 mmol) at 0° C. After stirred at 0° C. for 1 h, the reaction mixture was quenched by addition of water (30 mL) and extracted with ethyl acetate (20 mL×2). The combined organic layers were washed with brine (20 mL), dried over anhydrous sodium sulfate, filtered and the filtrate was concentrated under reduced pressure. The residue was purified by column chromatography to afford the title compound. LCMS R_{T}=2.433 min, m/z=475.0 [M+H]⁺

### Step 3: (2R,4S)-tert-butyl 4-(8-(2-(((tert-butyldimethylsilyl)oxy)methyl)thieno[3,2-b]pyridin-7-yl)-6-chloro-3,4-dihydroquinolin-1(2H)-yl)-2-(2-hydroxypropan-2-yl)pyrrolidine-1-carboxylate

To a solution of [2-[[tert-butyl(dimethyl)silyl]oxymethyl]thieno[3,2-b]pyridin-7-yl]boronic acid (256 mg, 0.8 mmol) and tert-butyl (2R,4S)-4-(8-bromo-6-chloro-3,4-dihydro-2H-quinolin-1-yl)-2-(1-hydroxy-1-methyl-ethyl)pyrrolidine-1-carboxylate (250 mg, 0.5 mmol) in dioxane (1 mL), water (0.1 mL) was added cesium carbonate (344 mg, 1.06 mmol), dichloro[1,1′-bis(diphenylphosphino)ferrocene]palladium(II) (77 mg, 0.1 mmol) at 20° C. After stirred at 100° C. for 3 h, the reaction mixture was quenched by addition of water (30 mL) and extracted with ethyl acetate (20 mL×2). The combined organic layers were washed with brine (20 mL), dried over anhydrous sodium sulfate, filtered and the filtrate was concentrated under reduced pressure. The residue was purified by column chromatography to afford the title compound. LCMS R_{T}=5.201 min, m/z=672.4 [M+H]⁺.

### Step 4: (2R,4S)-tert-butyl 4-(6-chloro-8-(2-(hydroxymethyl)thieno[3,2-b]pyridin-7-yl)-3,4-dihydroquinolin-1(2H)-yl)-2-(2-hydroxypropan-2-yl)pyrrolidine-1-carboxylate

To a solution of (2R,4S)-tert-butyl 4-(8-(2-(((tert-butyldimethylsilyl)oxy)methyl)thieno[3,2-b]pyridin-7-yl)-6-chloro-3,4-dihydroquinolin-1(2H)-yl)-2-(2-hydroxypropan-2-yl)pyrrolidine-1-carboxylate (200 mg, 0.3 mmol) in THF (2 mL) was added tetrabutylammonium fluoride (1 M in THF, 0.3 mL) at 20° C. After stirred at 20° C. for 1 h, the reaction mixture was concentrated under reduced pressure. The residue was purified by column chromatography to afford the title compound. LCMS R_{T}=0.533 min, m/z=558.2 [M+H]⁺

### Step 5

Example 189 was prepared from succinimide and tert-butyl (2R,4S)-4-[6-chloro-8-[2-(hydroxymethyl)thieno[3,2-b]pyridin-7-yl]-3,4-dihydro-2H-quinolin-1-yl]-2-(1-hydroxy-1-methyl-ethyl)pyrrolidine-1-carboxylate, following the procedure described in the synthesis of Example 54. The product was purified by preparative HPLC. ¹H NMR (400 MHz, CD₃OD) δ=8.79-8.67 (m, 1H), 8.52 (s, 1H), 7.53-7.42 (m, 2H), 7.19 (q, J=2.4 Hz, 2H), 4.97 (s, 2H), 3.75-3.60 (m, 1H), 3.30 (br. d, J=1.6 Hz, 3H), 2.80-2.60 (s, 8H), 2.11-1.35 (m, 4H), 1.08 (s, 3H), 0.96-0.70 (m, 3H). LCMS R_{T}=1.535 min, m/z=539.2 [M+H]⁺

### Example 190: 8-[2-[(4-tert-butyl-6-oxo-pyridazin-1-yl)methyl]thieno[3,2-b]pyridin-7-yl]-1-[(3S,5R)-5-(hydroxymethyl)pyrrolidin-3-yl]-3,4-dihydro-2H-quinoline-6-carbonitrile, formic acid salt

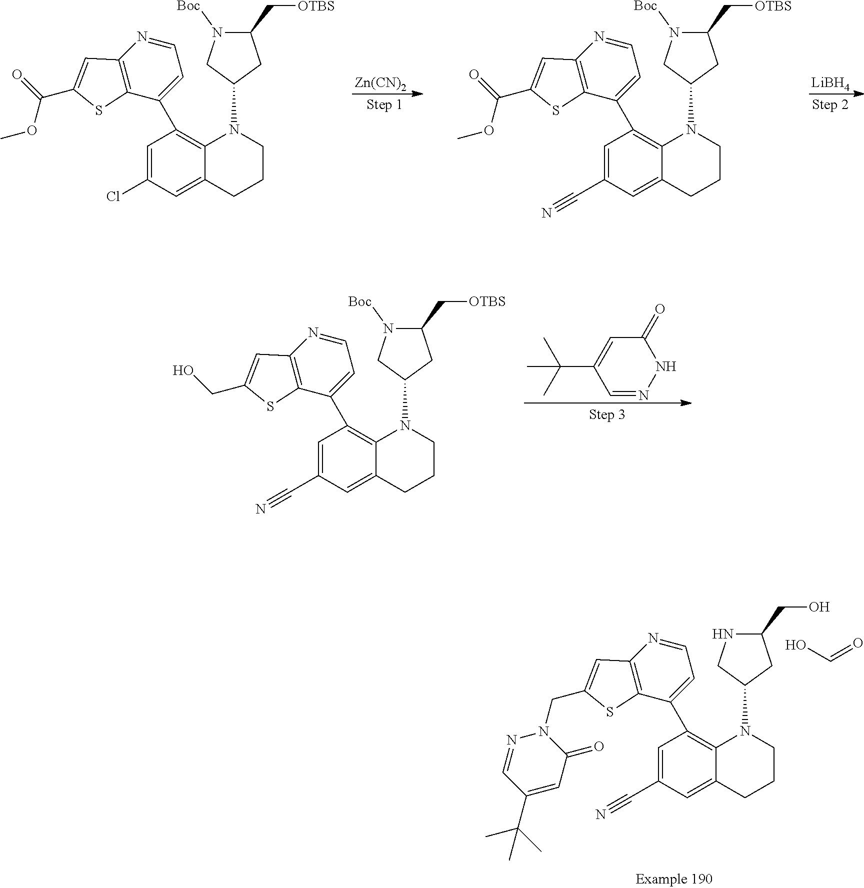

### Step 1: methyl 7-[1-[(3S,5R)-1-tert-butoxycarbonyl-5-[[tert-butyl(dimethyl)silyl]oxymethyl]pyrrolidin-3-yl]-6-cyano-3,4-dihydro-2H-quinolin-8-yl]thieno[3,2-b]pyridine-2-carboxylate

To a solution of methyl 7-[1-[(3S,5R)-1-tert-butoxycarbonyl-5-[[tert-butyl(dimethyl)silyl]oxymethyl]pyrrolidin-3-yl]-6-chloro-3,4-dihydro-2H-quinolin-8-yl]thieno[3,2-b]pyridine-2-carboxylate (300 mg, 0.45 mmol) in N-methylpyrrolidone (5 mL) was added Bis(tri-tert-butylphosphine)palladium(0) (46 mg, 0.09 mmol) and zinc cyanide (524 mg, 4.46 mmol) at 25° C. After stirred at 170° C. for 1 h, the reaction mixture was quenched by water 10 ml, and then extracted with ethyl acetate (10 mL×3). The combined organic layers were dried over sodium sulphate, filtered and the filtrate was concentrated under reduced pressure to give a residue. The residue was purified by column chromatography to afford the title compound. LCMS R_{T}=2.061 min, m/z=663.1 [M+H]⁺.

### Step 2: (2R,4S)-tert-butyl 2-(((tert-butyldimethylsilyl)oxy)methyl)-4-(6-cyano-8-(2-(hydroxymethyl)thieno[3,2-b]pyridin-7-yl)-3,4-dihydroquinolin-1(2H)-yl)pyrrolidine-1-carboxylate

To a solution of methyl 7-[1-[(3S,5R)-1-tert-butoxycarbonyl-5-[[tert-butyl(dimethyl)silyl]oxymethyl]pyrrolidin-3-yl]-6-cyano-3,4-dihydro-2H-quinolin-8-yl]thieno[3,2-b]pyridine-2-carboxylate (200 mg, 0.30 mmol) in THF (5 mL) was added lithium borohydride (132 mg, 6.03 mmol) at 0° C. After stirred at 25° C. for 12 h, the reaction mixture was quenched by addition ammonium chloride 10 mL, and then extracted with ethyl acetate (10 mL×3). The combined organic layers were dried over sodium sulphate, filtered and the filtrate was concentrated under reduced pressure to give a residue. The residue was purified by column chromatography to afford the title compound. LCMS R_{T}=1.849 min, m/z=635.4 [M+H]⁺.

### Step 3

Example 190 was prepared from (2R,4S)-tert-butyl 2-(((tert-butyldimethylsilyl)oxy)methyl)-4-(6-cyano-8-(2-(hydroxymethyl)thieno[3,2-b]pyridin-7-yl)-3,4-dihydroquinolin-1(2H)-yl)pyrrolidine-1-carboxylate and 4-tert-butyl-1H-pyridazin-6-one, following the procedure described in the synthesis of Example 108. The product was purified by preparative HPLC. ¹H NMR (400 MHz, CD₃OD) δ=8.72 (d, J=4.8 Hz, 1H), 8.14 (d, J=2.4 Hz, 1H), 7.55 (s, 1H), 7.50-7.41 (m, 3H), 6.83 (d, J=2.4 Hz, 1H), 5.60 (d, J=9.2 Hz, 2H), 5.65-5.54 (m, 1H), 3.51-3.34 (m, 2H), 3.27 (s, 3H), 3.08-2.45 (m, 4H), 2.12-1.39 (m, 4H), 1.30 (s, 9H). LCMS R_{T}=1.615 min, m/z=555.3 [M+H]⁺.

### Example 191: 8-(2-((3-(3,3-difluorocyclobutyl)-2,6-dioxo-2,3-dihydropyrimidin-1(6H)-yl)methyl)thieno[3,2-b]pyridin-7-yl)-1-((3S,5R)-5-(hydroxymethyl)pyrrolidin-3-yl)-1,2,3,4-tetrahydroquinoline-6-carbonitrile, formic acid salt

Example 191 was prepared from 1-(3,3-difluorocyclobutyl)pyrimidine-2,4-dione and (2R,4S)-tert-butyl 2-(((tert-butyldimethylsilyl)oxy)methyl)-4-(6-cyano-8-(2-(hydroxymethyl)thieno[3,2-b]pyridin-7-yl)-3,4-dihydroquinolin-1(2H)-yl)pyrrolidine-1-carboxylate, following the procedure described in the synthesis of Example 108. The product was purified by preparative HPLC. ¹H NMR (400 MHz, CD₃OD) δ=8.78-8.66 (m, 1H), 7.70-7.61 (m, 1H), 7.58-7.37 (m, 4H), 5.87-5.76 (m, 1H), 5.46-5.26 (m, 2H), 4.71-4.53 (m, 1H), 4.09-3.79 (m, 1H), 3.67-3.36 (m, 2H), 3.29-2.55 (m, 11H), 2.21-1.31 (m, 4H). LCMS R_{T}=1.075 min, m/z=605.4 [M+H]⁺

### Example 192a and 192b: (1aS,7bR)-4-(2-((4-(tert-butyl)-6-oxopyridazin-1(6H)-yl)methyl)thieno[3,2-b]pyridin-7-yl)-3-((3S,5R)-5-(hydroxymethyl)pyrrolidin-3-yl)-1a,2,3,7b-tetrahydro-1H-cyclopropa[c]quinoline-6-carbonitrile, formic acid salt and (1aR,7bS)-4-(2-((4-(tert-butyl)-6-oxopyridazin-1(6H)-yl)methyl)thieno[3,2-b]pyridin-7-yl)-3-((3S,5R)-5-(hydroxymethyl)pyrrolidin-3-yl)-1a,2,3,7b-tetrahydro-1H-cyclopropa[c]quinoline-6-carbonitrile, formic acid salt

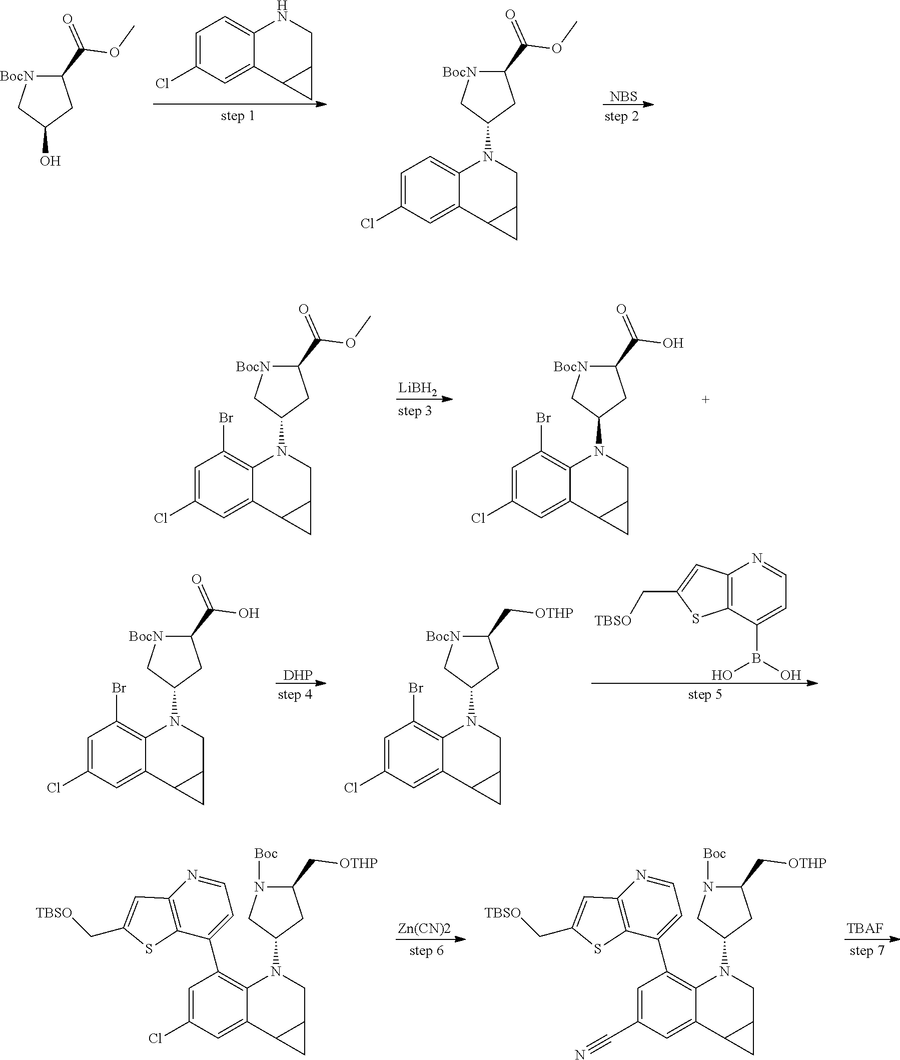

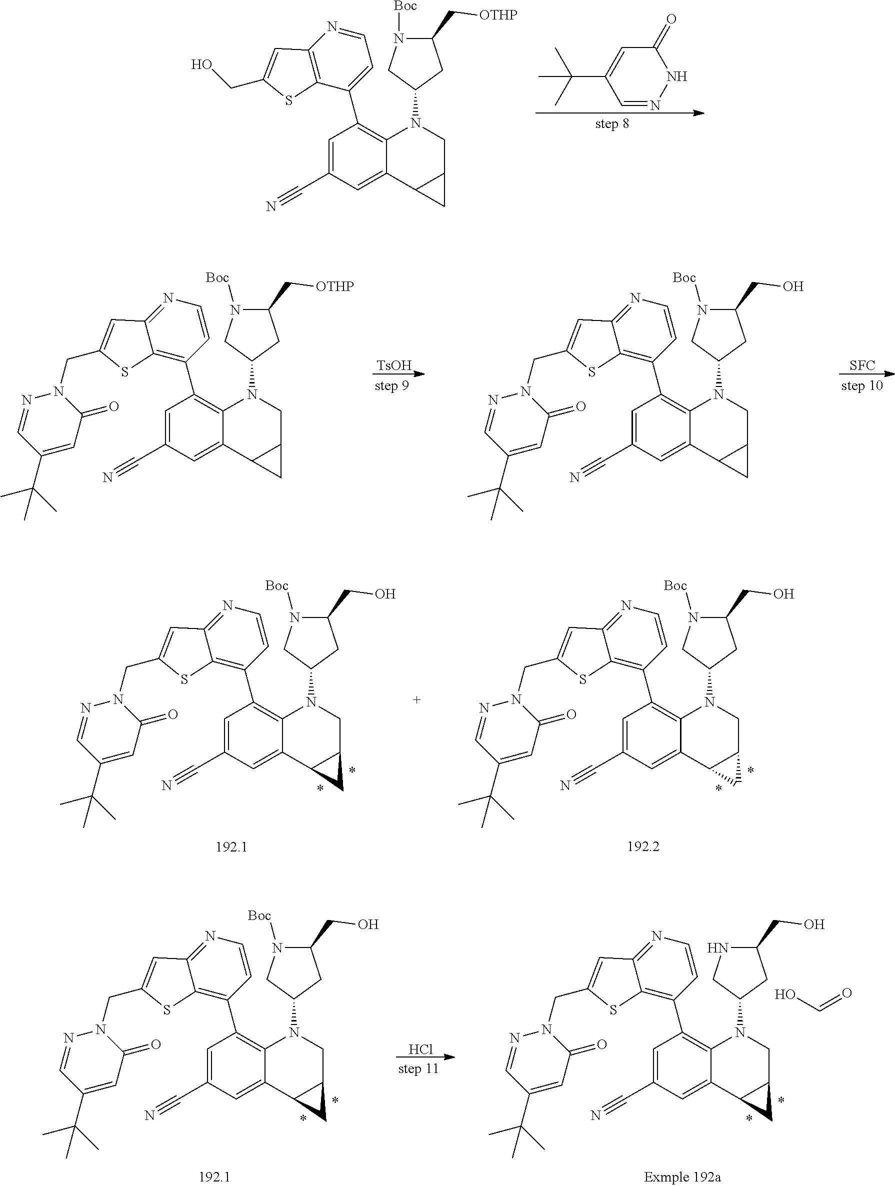

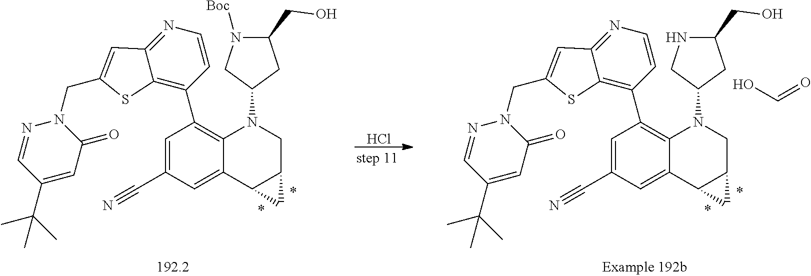

### Step 1: (2R,4S)-1-tert-butyl 2-methyl 4-(6-chloro-1a,2-dihydro-1H-cyclopropa[c]quinolin-3(7bH)-yl)pyrrolidine-1,2-dicarboxylate

To a solution of (2R,4R)-1-tert-butyl 2-methyl 4-hydroxypyrrolidine-1,2-dicarboxylate (1.50 g, 6.12 mmol) in dichloromethane (30 mL) was added diisopropylethylamine (1.08 g, 8.35 mmol) and Trifluoromethanesulfonic anhydride (1.88 g, 6.68 mmol) at −65° C. The mixture was warmed to −30° C. and was added 6-chloro-1a,2,3,7b-tetrahydro-1H-cyclopropa[c]quinoline (1 g, 5.57 mmol) in dichloromethane (10 mL). After stirred at 25° C. for 2 h, the mixture was quenched by addition of water (50 mL) and extracted with ethyl acetate (30 mL×3). The combined organic layers were washed with brine (20 mL), dried over anhydrous sodium sulfate, filtered and the filtrate was concentrated under reduced pressure. The residue was purified by column chromatography to afford the title compound. LCMS R_{T}=1.863 min, m/z=407.1 [M+H]⁺.

### Step 2: (2R,4S)-1-tert-butyl 2-methyl 4-(4-bromo-6-chloro-1a,2-dihydro-1H-cyclopropa[c]quinolin-3(7bH)-yl)pyrrolidine-1,2-dicarboxylate

To a solution of (2R,4S)-1-tert-butyl 2-methyl 4-(6-chloro-1a,2-dihydro-1H-cyclopropa[c]quinolin-3(7bH)-yl)pyrrolidine-1,2-dicarboxylate (1.2 g, 2.95 mmol) in acetic acid (10 mL) was added 1,3-dibromo-5,5-dimethyl-imidazolidine-2,4-dione (506 mg, 1.77 mmol) at 0° C. After stirred at 0° C. for 10 min, the mixture was quenched by addition of saturated aqueous sodium bicarbonate (50 mL) and extracted with ethyl acetate (30 mL×3). The combined organic layers were washed with brine (20 mL), dried over anhydrous sodium sulfate, filtered and the filtrate was concentrated under reduced pressure. The residue was purified by column chromatography to afford the title compound. LCMS R_{T}=2.246 min, m/z=487.2 [M+H]⁺.

### Step 3: (2R,4S)-tert-butyl 4-(4-bromo-6-chloro-1a,2-dihydro-1H-cyclopropa[c]quinolin-3(7bH)-yl)-2-(hydroxymethyl)pyrrolidine-1-carboxylate and (2R,4R)-tert-butyl 4-(4-bromo-6-chloro-1a,2-dihydro-1H-cyclopropa[c]quinolin-3(7bH)-yl)-2-(hydroxymethyl)pyrrolidine-1-carboxylate

To a solution of (2R,4S)-1-tert-butyl 2-methyl 4-(4-bromo-6-chloro-1a,2-dihydro-1H-cyclopropa[c]quinolin-3(7bH)-yl)pyrrolidine-1,2-dicarboxylate (1 g, 2.06 mmol) in THF (20 mL) was added lithium borohydride (200.00 mg, 9.18 mmol) at 0° C. After stirred at 0° C. for 2 h, the mixture was quenched by addition of saturated aqueous ammonium chloride (40 mL) and extracted with ethyl acetate (20 mL×3). The combined organic layers were washed with brine (20 mL), dried over anhydrous sodium sulfate, filtered and the filtrate was concentrated under reduced pressure. The residue was purified by column chromatography to afford first eluting fraction (LCMS R_{T}=1.557 min, m/z=459.2 [M+H]+) and second eluting fraction (LCMS R_{T}=1,653 min, m/z=457.2 [M+H]+).

### Step 4: tert-butyl (2R,4S)-4-(4-bromo-6-chloro-1,1a,2,7b-tetrahydrocyclopropa[c]quinolin-3-yl)-2-(tetrahydropyran-2-yloxymethyl)pyrrolidine-1-carboxylate

To a solution of tert-butyl (2R,4S)-4-(4-bromo-6-chloro-1,1a,2,7b-tetrahydrocyclopropa[c]quinolin-3-yl)-2-(hydroxymethyl)pyrrolidine-1-carboxylate (350 mg, 0.76 mmol) in acetone (3 mL) was added 2,3-dihydropyran (129 mg, 1.53 mmol), Pyridinium 4-toluenesulfonate (58 mg, 0.23 mmol) at 20° C. After stirred at 50° C. for 3 h, the mixture was concentrated under reduced pressure. The residue was purified by column chromatography to afford the title compound. LCMS R_{T}=0.713 min, m/z=543.2 [M+H]⁺.

### Step 5: tert-butyl (2R,4S)-4-[4-[2-[[tert-butyl(dimethyl)silyl]oxymethyl]thieno[3,2-b]pyridin-7-yl]-6-chloro-1,1a,2,7b-tetrahydrocyclopropa[c]quinolin-3-yl]-2-(tetrahydropyran-2-yloxymethyl)pyrrolidine-1-carboxylate

To a solution of [2-[[tert-butyl(dimethyl)silyl]oxymethyl]thieno[3,2-b]pyridin-7-yl]boronic acid (358 mg, 1.11 mmol) and tert-butyl (2R,4S)-4-(4-bromo-6-chloro-1,1a,2,7b-tetrahydrocyclopropa[c]quinolin-3-yl)-2-(tetrahydropyran-2-yloxymethyl)pyrrolidine-1-carboxylate (400 mg, 0.74 mmol) in dioxane (1 mL), Water (0.1 mL) was added cesium carbonate (722 mg, 2.21 mmol), dichloro[1,1′-bis(diphenylphosphino)ferrocene]palladium(II) (54 mg, 0.07 mmol) at 20° C. After stirred at 100° C. for 3 h, the mixture was concentrated under reduced pressure. The residue was purified by column chromatography to afford the title compound. LCMS R_{T}=0.679 min, m/z=740.5 [M+H]⁺.

### Step 6: tert-butyl (2R,4S)-4-[4-[2-[[tert-butyl(dimethyl)silyl]oxymethyl]thieno[3,2-b]pyridin-7-yl]-6-cyano-1,1a,2,7b-tetrahydrocyclopropa[c]quinolin-3-yl]-2-(tetrahydropyran-2-yloxymethyl)pyrrolidine-1-carboxylate

To a solution of tert-butyl (2R,4S)-4-[4-[2-[[tert-butyl(dimethyl)silyl]oxymethyl]thieno[3,2-b]pyridin-7-yl]-6-chloro-1,1a,2,7b-tetrahydrocyclopropa[c]quinolin-3-yl]-2-(tetrahydropyran-2-yloxymethyl)pyrrolidine-1-carboxylate (900 mg, 1.22 mmol) and diisopropylethylamine (471 mg, 3.65 mmol) in dioxane (10 mL) was added zinc cyanide (285 mg, 2.43 mmol), Bis(tri-tert-butylphosphine)palladium(0) (124 mg, 0.24 mmol) at 20° C. After stirred at 90° C. for 12 h, the mixture was quenched by addition of water (60 mL) and extracted with ethyl acetate (20 mL×3). The combined organic layers were washed with brine (20 mL), dried over anhydrous sodium sulfate, filtered and the filtrate was concentrated under reduced pressure. The residue was purified by column chromatography to afford the title compound. LCMS R_{T}=0.664 min, m/z=731.5 [M+H]⁺.

### Step 7: tert-butyl (2R,4S)-4-[6-cyano-4-[2-(hydroxymethyl)thieno[3,2-b]pyridin-7-yl]-1,1a,2,7b-tetrahydrocyclopropa[c]quinolin-3-yl]-2-(tetrahydropyran-2-yloxymethyl)pyrrolidine-1-carboxylate

To a solution of tert-butyl (2R,4S)-4-[4-[2-[[tert-butyl(dimethyl)silyl]oxymethyl]thieno[3,2-b]pyridin-7-yl]-6-cyano-1,1a,2,7b-tetrahydrocyclopropa[c]quinolin-3-yl]-2-(tetrahydropyran-2-yloxymethyl)pyrrolidine-1-carboxylate (260 mg, 0.35 mmol) in THF (5 mL) was added tetrabutylammonium fluoride (1 M, 0.4 mL) at 20° C. After stirred at 20° C. for 1 h, the mixture was concentrated under reduced pressure. The residue was purified by column chromatography to afford the title compound. LCMS R_{T}=0.535 min, m/z=617.4 [M+H]⁺.

### Step 8: tert-butyl (2R,4S)-4-[4-[2-[(4-tert-butyl-6-oxo-pyridazin-1-yl)methyl]thieno[3,2-b]pyridin-7-yl]-6-cyano-1,1a,2,7b-tetrahydrocyclopropa[c]quinolin-3-yl]-2-(tetrahydropyran-2-yloxymethyl)pyrrolidine-1-carboxylate

To a solution of tert-butyl (2R,4S)-4-[6-cyano-4-[2-(hydroxymethyl)thieno[3,2-b]pyridin-7-yl]-1,1a,2,7b-tetrahydrocyclopropa[c]quinolin-3-yl]-2-(tetrahydropyran-2-yloxymethyl)pyrrolidine-1-carboxylate (160 mg, 0.26 mmol) and 4-tert-butyl-1H-pyridazin-6-one (79 mg, 0.52 mmol) in toluene (1 mL) was added tetramethylazodicarboxamide (134 mg, 0.78 mmol), tributylphosphane (315 mg, 1.56 mmol) at 20° C. After stirred at 100° C. for 1 h, the mixture was concentrated under reduced pressure. The residue was purified by column chromatography to afford the title compound. LCMS R_{T}=0.604 min, m/z=751.5 [M+H]⁺.

### Step 9: tert-butyl (2R,4S)-4-[4-[2-[(4-tert-butyl-6-oxo-pyridazin-1-yl)methyl]thieno[3,2-b]pyridin-7-yl]-6-cyano-1,1a,2,7b-tetrahydrocyclopropa[c]quinolin-3-yl]-2-(hydroxymethyl)pyrrolidine-1-carboxylate

To a solution of tert-butyl (2R,4S)-4-[4-[2-[(4-tert-butyl-6-oxo-pyridazin-1-yl)methyl]thieno[3,2-b]pyridin-7-yl]-6-cyano-1,1a,2,7b-tetrahydrocyclopropa[c]quinolin-3-yl]-2-(tetrahydropyran-2-yloxymethyl)pyrrolidine-1-carboxylate (190 mg, 0.25 mmol) in methyl alcohol (1 mL) was added p-toluenesulfonic acid (44 mg, 0.2 mmol) at 20° C. After stirred at 20° C. for 1 h, the mixture was quenched by addition of water (30 mL) and extracted with ethyl acetate (10 mL×3). The combined organic layers were washed with brine (20 mL), dried over anhydrous sodium sulfate, filtered and the filtrate was concentrated under reduced pressure. The residue was purified by column chromatography to afford the title compound. LCMS R_{T}=0.541 min, m/z=667.4 [M+H]⁺.

### Step 10: (2R,4S)-tert-butyl 4-((1aS,7bR)-4-(2-((4-(tert-butyl)-6-oxopyridazin-1(6H)-yl)methyl)thieno[3,2-b]pyridin-7-yl)-6-cyano-1a,2-dihydro-1H-cyclopropa[c]quinolin-3(7bH)-yl)-2-(hydroxymethyl)pyrrolidine-1-carboxylate and (2R,4S)-tert-butyl 4-((1aR,7bS)-4-(2-((4-(tert-butyl)-6-oxopyridazin-1(6H)-yl)methyl)thieno[3,2-b]pyridin-7-yl)-6-cyano-1a,2-dihydro-1H-cyclopropa[c]quinolin-3(7bH)-yl)-2-(hydroxymethyl)pyrrolidine-1-carboxylate

The above racemic product was separated by SFC to give first eluting fraction (192.1, Rt=4.136 min; LCMS RT=0.544 min, m/z=667.4 [M+H]+) and second eluting fraction (192.2, Rt=5.545 min; LCMS RT=0.544 min, m/z=667.4 [M+H]+).

### Step 11

A solution of 192.1 (30 mg, 0.05 mmol) in hydrochloric acid in dioxane (4 M, 1 mL) was stirred at 20° C. for 1 h. The mixture was concentrated under reduced pressure. The residue was purified by preparative HPLC to afford Example 195a. ¹H NMR (400 MHz, CD₃OD) δ=8.73-8.45 (m, 1H), 8.05 (d, J=2.0 Hz, 1H), 7.57-7.01 (m, 4H), 6.73 (s, 1H), 5.63-5.27 (m, 2H), 3.56-3.40 (m, 1H), 3.21-2.83 (m, 3H), 2.79-2.38 (m, 4H), 2.12-1.84 (m, 2H), 1.68-1.02 (m, 10H), 0.98-0.22 (m, 3H). LCMS R_{T}=1.323 min, m/z=567.5 [M+H]⁺.

A solution of 192.2 (30 mg, 0.045 mmol) in hydrochloric acid in dioxane (4 M, 2 mL) was stirred at 20° C. for 1 h. The mixture was concentrated under reduced pressure. The residue was purified by preparative HPLC to afford Example 195b. ¹H NMR (400 MHz, CD₃OD) δ=8.74 (br d, J=4.0 Hz, 1H), 8.53 (s, 1H), 8.15 (d, J=1.6 Hz, 1H), 7.72-7.26 (m, 4H), 6.83 (d, J=1.6 Hz, 1H), 5.76-5.41 (m, 2H), 4.13-3.91 (m, 1H), 3.70-3.36 (m, 2H), 3.26 (br d, J=5.6 Hz, 2H), 2.50-1.87 (m, 4H), 1.76-1.52 (m, 1H), 1.31 (s, 9H), 1.22-1.04 (m, 2H), 0.94 (br s, 1H). LCMS R_{T}=1.323 min, m/z=567.5 [M+H]⁺. (*absolute configuration was not determined.)

### Example 193a and 193b: (1aS,7bR)-4-(2-((4-(tert-butyl)-6-oxopyridazin-1(6H)-yl)methyl)thieno[3,2-b]pyridin-7-yl)-3-((3R,5R)-5-(hydroxymethyl)pyrrolidin-3-yl)-1a,2,3,7b-tetrahydro-1H-cyclopropa[c]quinoline-6-carbonitrile, formic acid salt and

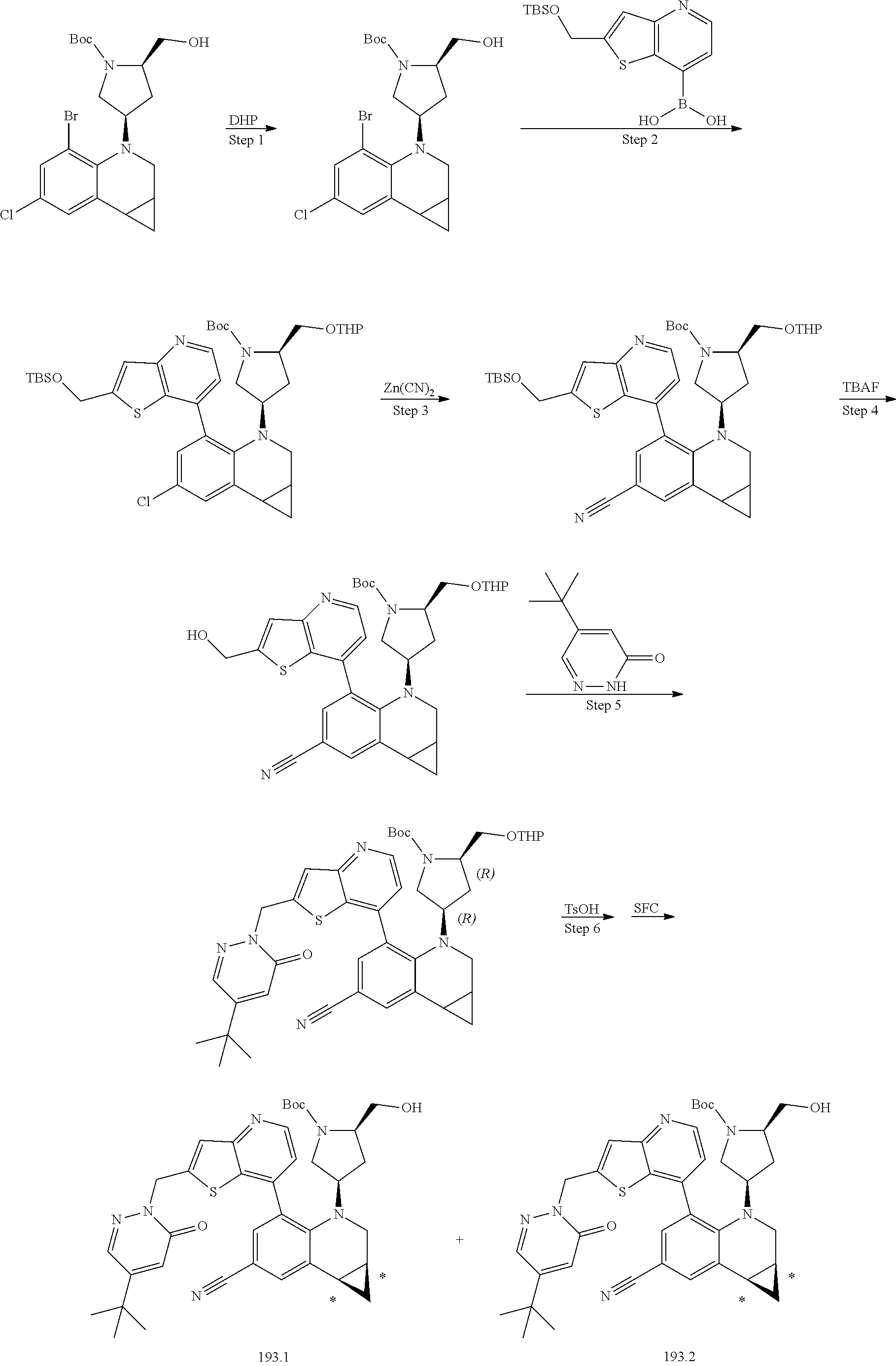

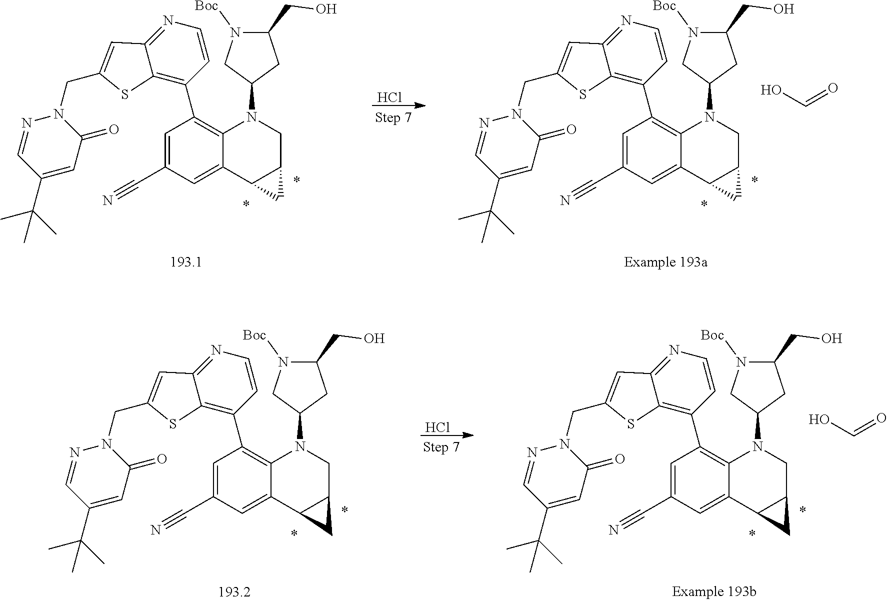

### Step 1: (2R,4R)-tert-butyl 4-(4-bromo-6-chloro-1a,2-dihydro-1H-cyclopropa[c]quinolin-3(7bH)-yl)-2-(((tetrahydro-2H-pyran-2-yl)oxy)methyl)pyrrolidine-1-carboxylate

To a solution of (2R,4R)-tert-butyl 4-(4-bromo-6-chloro-1a,2-dihydro-1H-cyclopropa[c]quinolin-3(7bH)-yl)-2-(hydroxymethyl)pyrrolidine-1-carboxylate (400 mg, 0.87 mmol) in acetone (4 mL) were added (3,4-dihydro-2H-pyran-2-yl)methanol (220 mg, 2.62 mmol) and Pyridinium p-toluenesulfonate (66 mg, 0.26 mmol). After stirred at 50° C. for 2 h, the reaction mixture was concentrated under reduced pressure. The residue was purified by column chromatography to give the title compound. LCMS R_{T}=0.727 min, m/z=543.2 [M+2+H]⁺.

### Step 2: (2R,4R)-tert-butyl 4-(4-(2-(((tert-butyldimethylsilyl)oxy)methyl)thieno[3,2-b]pyridin-7-yl)-6-chloro-1a,2-dihydro-1H-cyclopropa[c]quinolin-3(7bH)-yl)-2-(((tetrahydro-2H-pyran-2-yl)oxy)methyl)pyrrolidine-1-carboxylate

To a solution of (2R,4R)-tert-butyl 4-(4-bromo-6-chloro-1a,2-dihydro-1H-cyclopropa[c]quinolin-3(7bH)-yl)-2-(((tetrahydro-2H-pyran-2-yl)oxy)methyl)pyrrolidine-1-carboxylate (230 mg, 0.42 mmol) in dioxane (2 mL) and water (0.2 mL) were added cesium carbonate (277 mg, 0.85 mmol), [2-[[tert-butyl(dimethyl)silyl]oxymethyl]thieno[3,2-b]pyridin-7-yl]boronic acid (137 mg, 0.42 mmol) and Dichloro[1,1′-bis(diphenylphosphino)ferrocene]palladium(II) (62 mg, 0.08 mmol). After stirred at 120° C. for 2 h, the residue was diluted with saturated ammonium chloride (15 mL), extracted with dichloromethane (15 mL×3). The combined organic layers were washed with brine (15 mL), dried over anhydrous sodium sulfate and concentrated under reduced pressure. The residue was purified by column chromatography to give the title compound. LCMS R_{T}=0.653 min, m/z=740.5 [M+H]⁺.

### Step 3: (2R,4R)-tert-butyl 4-(4-(2-(((tert-butyldimethylsilyl)oxy)methyl)thieno[3,2-b]pyridin-7-yl)-6-cyano-1a,2-dihydro-1H-cyclopropa[c]quinolin-3(7bH)-yl)-2-(((tetrahydro-2H-pyran-2-yl)oxy)methyl)pyrrolidine-1-carboxylate

To a solution of (2R,4R)-tert-butyl 4-(4-(2-(((tert-butyldimethylsilyl)oxy)methyl)thieno[3,2-b]pyridin-7-yl)-6-chloro-1a,2-dihydro-1H-cyclopropa[c]quinolin-3(7bH)-yl)-2-(((tetrahydro-2H-pyran-2-yl)oxy)methyl)pyrrolidine-1-carboxylate (330 mg, 0.45 mmol) in dioxane (3 mL) were added zinc cyanide (680 mg, 5.79 mmol) and bis(tri-tert-butylphosphine) palladium(0) (46 mg, 0.09 mmol). After stirred at 95° C. for 12 h, the reaction mixture was diluted with water (20 mL) and extracted with ethyl acetate (20 mL×3). The combined organic layers were washed with brine (20 mL), dried over anhydrous sodium sulfate, filtered and the filtrate was concentrated under reduced pressure. The residue was purified by column chromatography to afford the title compound. LCMS R_{T}=3.100 min, m/z=731.5 [M+H]⁺.

### Step 4: (2R,4R)-tert-butyl 4-(6-cyano-4-(2-(hydroxymethyl)thieno[3,2-b]pyridin-7-yl)-1a,2-dihydro-1H-cyclopropa[c]quinolin-3(7bH)-yl)-2-(((tetrahydro-2H-pyran-2-yl)oxy)methyl)pyrrolidine-1-carboxylate

To a solution of (2R,4R)-tert-butyl 4-(4-(2-(((tert-butyldimethylsilyl)oxy)methyl)thieno[3,2-b]pyridin-7-yl)-6-cyano-1a,2-dihydro-1H-cyclopropa[c]quinolin-3(7bH)-yl)-2-(((tetrahydro-2H-pyran-2-yl)oxy)methyl)pyrrolidine-1-carboxylate (250 mg, 0.34 mmol) in THF (10 mL) were added tetrabutylammonium fluoride (0.5 mL, 1.0 M in THF). After stirred for 0.5 h at 25° C., the residue was concentrated under reduced pressure. The residue was purified by column chromatography to give the title compound. LCMS R_{T}=0.538 min, m/z=617.3 [M+H]⁺.

### Step 5: (2R,4R)-tert-butyl 4-(4-(2-((4-(tert-butyl)-6-oxopyridazin-1(6H)-yl)methyl)thieno[3,2-b]pyridin-7-yl)-6-cyano-1a,2-dihydro-1H-cyclopropa[c]quinolin-3(7bH)-yl)-2-(((tetrahydro-2H-pyran-2-yl)oxy)methyl)pyrrolidine-1-carboxylate

To a solution of (2R,4R)-tert-butyl 4-(6-cyano-4-(2-(hydroxymethyl)thieno[3,2-b]pyridin-7-yl)-1a,2-dihydro-1H-cyclopropa[c]quinolin-3(7bH)-yl)-2-(((tetrahydro-2H-pyran-2-yl)oxy)methyl)pyrrolidine-1-carboxylate (100 mg, 0.16 mmol) in toluene (2 mL) were added 4-tert-butyl-1H-pyridazin-6-one (37 mg, 0.24 mmol), (3E)-3-(dimethylcarbamoylimino)-1,1-dimethyl-urea (84 mg, 0.49 mmol) and tributylphosphane (197 mg, 0.97 mmol) at 25° C. After stirred at 100° C. for 1 h, the reaction mixture was concentrated under reduced pressure to remove the solvent. The residue was purified by preparative TLC to give the title compound. LCMS R_{T}=0.607 min, m/z=751.5 [M+H]⁺.

### Step 6: (2R,4R)-tert-butyl 4-((1aS,7bR)-4-(2-((4-(tert-butyl)-6-oxopyridazin-1(6H)-yl)methyl)thieno[3,2-b]pyridin-7-yl)-6-cyano-1a,2-dihydro-1H-cyclopropa[c]quinolin-3(7bH)-yl)-2-(hydroxymethyl)pyrrolidine-1-carboxylate and (2R,4R)-tert-butyl 4-((1aR,7bS)-4-(2-((4-(tert-butyl)-6-oxopyridazin-1(6H)-yl)methyl)thieno[3,2-b]pyridin-7-yl)-6-cyano-1a,2-dihydro-1H-cyclopropa[c]quinolin-3(7bH)-yl)-2-(hydroxymethyl)pyrrolidine-1-carboxylate

To a solution of (2R,4R)-tert-butyl 4-(4-(2-((4-(tert-butyl)-6-oxopyridazin-1(6H)-yl)methyl)thieno[3,2-b]pyridin-7-yl)-6-cyano-1a,2-dihydro-1H-cyclopropa[c]quinolin-3(7bH)-yl)-2-(((tetrahydro-2H-pyran-2-yl)oxy)methyl)pyrrolidine-1-carboxylate (120 mg, 0.16 mmol) in Methanol (3 mL) was added 4-methylbenzenesulfonic acid (55 mg, 0.32 mmol) at 25° C. After stirred at 25° C. for 0.5 h, the reaction mixture was concentrated under reduced pressure to remove the solvent. The residue was purified by preparative TLC.

The racemic product was separated by SFC to give first eluting fraction (193.1, Rt=1.510 min; LCMS R_{T}=0.535 min, m/z=667.4 [M+H]+) and second eluting fraction (193.2, Rt=4.649 min; LCMS R_{T}=0.543 min, m/z=667.4 [M+H]⁺.

### Step 7

To a solution of 193.1 (20 mg, 0.03 mmol) was dissolved in hydrochloric acid (4 mL, 4 M in dioxane). After stirred at 25° C. for 0.5 h, the reaction mixture was concentrated under reduced pressure. The residue was purified by preparative HPLC to give Example 193a. ¹H NMR (400 MHz, CD₃OD) δ=8.76 (d, J=4.0 Hz, 1H), 8.15 (d, J=2.0 Hz, 1H), 7.65-7.34 (m, 4H), 6.83-6.82 (m, 1H), 5.59 (q, J=14.8 Hz, 2H), 3.91-3.80 (m, 1H), 3.59-3.44 (m, 2H), 3.26 (s, 2H), 2.86 (s, 1H), 2.15-1.97 (m, 3H), 1.82-1.56 (m, 2H), 1.31 (s, 9H), 1.16-0.94 (m, 3H). LCMS R_{T}=1.677 min, m/z=567.4 [M+H]⁺.

To a solution of 193.2 (10 mg, 0.02 mmol) was dissolved in hydrochloric acid (4 mL, 4 M in dioxane). After stirred at 25° C. for 0.5 h, the reaction mixture was concentrated under reduced pressure. The residue was purified by preparative HPLC to give Example 193b. ¹H NMR (400 MHz, CD₃OD) δ=8.74 (s, 1H), 8.15 (d, J=2.0 Hz, 1H), 7.67-7.36 (m, 4H), 6.83 (d, J=1.6 Hz, 1H), 5.67-5.52 (m, 2H), 3.81 (s, 1H), 3.47 (d, J=10.0 Hz, 1H), 3.28 (s, 3H), 2.99-2.80 (m, 3H), 2.17-2.03 (m, 2H), 1.73-1.38 (m, 1H), 1.31 (s, 9H), 1.17-0.81 (m, 3H). LCMS R_{T}=1.640 min, m/z=567.4 [M+H]⁺. (*absolute configuration was not determined.)

### Example 194a and 194b: (1aS,7bR)-3-((3S,5R)-5-(hydroxymethyl)pyrrolidin-3-yl)-4-(2-((6-oxo-4-(1-(trifluoromethyl)cyclopropyl)pyridazin-1(6H)-yl)methyl)thieno[3,2-b]pyridin-7-yl)-1a,2,3,7b-tetrahydro-1H-cyclopropa[c]quinoline-6-carbonitrile, formic acid salt and (1aS,7bR)-3-((3S,5R)-5-(hydroxymethyl)pyrrolidin-3-yl)-4-(2-((6-oxo-4-(1-(trifluoromethyl)cyclopropyl)pyridazin-1(6H)-yl)methyl)thieno[3,2-b]pyridin-7-yl)-1a,2,3,7b-tetrahydro-1H-cyclopropa[c]quinoline-6-carbonitrile, formic acid salt

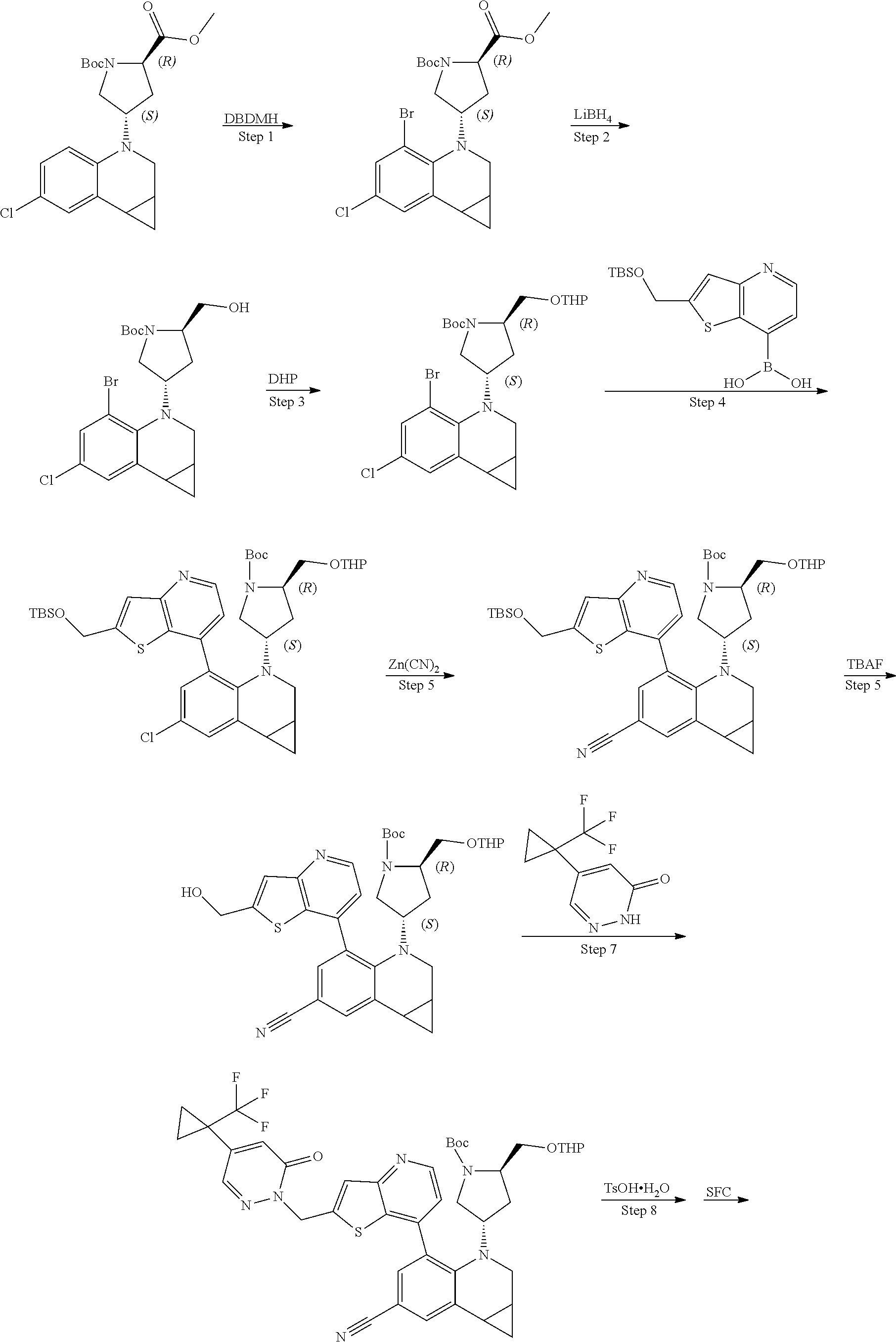

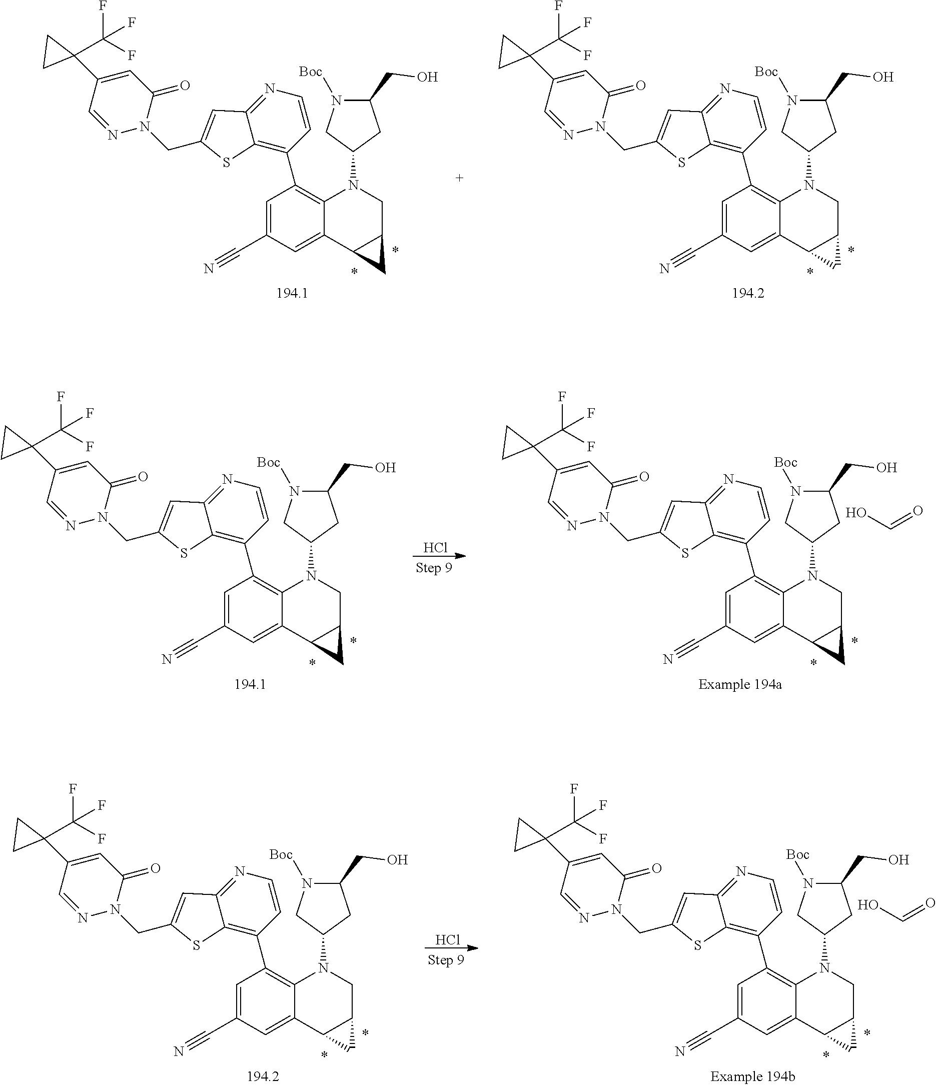

### Step 1: (2R,4S)-1-tert-butyl 2-methyl 4-(4-bromo-6-chloro-1a,2-dihydro-1H-cyclopropa[c]quinolin-3(7bH)-yl)pyrrolidine-1,2-dicarboxylate

To a solution of (2R,4S)-1-tert-butyl 2-methyl 4-(6-chloro-1a,2-dihydro-1H-cyclopropa[c]quinolin-3(7bH)-yl)pyrrolidine-1,2-dicarboxylate (5.4 g, 13.27 mmol) in acetic acid (50 mL) was added 1,3-dibromo-5,5-dimethyl-imidazolidine-2,4-dione (1.9 g, 6.64 mmol). After stirred at 25° C. for 1 h, the mixture was concentrated under reduced pressure. The residue was quenched by addition of saturated sodium bicarbonate (150 mL) and extracted with ethyl acetate (100 mL×3). The combined organic layers were washed with brine (250 mL), dried over anhydrous sodium sulfate and concentrated under reduced pressure. The residue was purified by column chromatography to afford the title compound. LCMS R_{T}=2.237 min, m/z=487.2 [M+H]⁺.

### Step 2: (2R,4S)-tert-butyl 4-(5-bromo-7-chloro-2H-benzo[b][1,4]oxazin-4(3H)-yl)-2-(hydroxymethyl)-2-methylpyrrolidine-1-carboxylate

To a solution of (2R,4S)-1-tert-butyl 2-methyl 4-(4-bromo-6-chloro-1a,2-dihydro-1H-cyclopropa[c]quinolin-3(7bH)-yl)pyrrolidine-1,2-dicarboxylate (2.6 g, 5.43 mmol) in THF (30 mL) was added lithium borohydride (521 mg, 23.91 mmol) at 0° C. After stirred at 20° C. for 12 h, the mixture was quenched by addition of saturated ammonium chloride (20 mL) and extracted with ethyl acetate (20 mL×3). The combined organic layers were washed with brine (30 mL), dried over anhydrous sodium sulfate and concentrated under reduced pressure. The residue was purified by column chromatography to afford the title compound. LCMS R_{T}=1.139 min, m/z=458.9 [M+H]⁺.

### Step 3: (2R,4S)-tert-butyl 4-(4-bromo-6-chloro-1a,2-dihydro-1H-cyclopropa[c]quinolin-3(7bH)-yl)-2-(((tetrahydro-2H-pyran-2-yl)oxy)methyl)pyrrolidine-1-carboxylate

To a solution of (2R,4S)-tert-butyl 4-(4-bromo-6-chloro-1a,2-dihydro-1H-cyclopropa[c]quinolin-3(7bH)-yl)-2-(hydroxymethyl)pyrrolidine-1-carboxylate (1.2 g, 2.51 mmol) in acetone (20 mL) were added (3,4-dihydro-2H-pyran-2-yl)methanol (634 mg, 7.54 mmol) and pyridinium p-toluenesulfonate (189 mg, 0.75 mmol). After stirred at 50° C. for 3 h, the mixture was concentrated under reduced pressure. The residue was purified by column chromatography to afford the title compound. LCMS R_{T}=1.067 min, m/z=541.2 [M+H]⁺.

### Step 4: (2R,4S)-tert-butyl 4-(4-(2-(((tert-butyldimethylsilyl)oxy)methyl)thieno[3,2-b]pyridin-7-yl)-6-chloro-1a,2-dihydro-1H-cyclopropa[c]quinolin-3(7bH)-yl)-2-(((tetrahydro-2H-pyran-2-yl)oxy)methyl)pyrrolidine-1-carboxylate

To a solution of (2R,4S)-tert-butyl 4-(4-bromo-6-chloro-1a,2-dihydro-1H-cyclopropa[c]quinolin-3(7bH)-yl)-2-(((tetrahydro-2H-pyran-2-yl)oxy)methyl)pyrrolidine-1-carboxylate (1.3 g, 2.40 mmol) in dioxane (30 mL) and water (3 mL) was added potassium carbonate (663 mg, 4.80 mmol), (2-(((tert-butyldimethylsilyl)oxy)methyl)thieno[3,2-b]pyridin-7-yl)boronic acid (1.2 g, 3.40 mmol) and dichloro[1,1′-bis(diphenylphosphino)ferrocene]palladium(II) (351 mg, 0.48 mmol). After stirred at 95° C. for 3 h, the mixture was filtered and the filtrate was concentrated under reduced pressure. The residue was purified by column chromatography to give the title compound. LCMS R_{T}=3.508 min, m/z=740.3 [M+H]⁺.

### Step 5: (2R,4S)-tert-butyl 4-(4-(2-(((tert-butyldimethylsilyl)oxy)methyl)thieno[3,2-b]pyridin-7-yl)-6-cyano-1a,2-dihydro-1H-cyclopropa[c]quinolin-3(7bH)-yl)-2-(((tetrahydro-2H-pyran-2-yl)oxy)methyl)pyrrolidine-1-carboxylate

To a solution of (2R,4S)-tert-butyl 4-(4-(2-(((tert-butyldimethylsilyl)oxy)methyl)thieno[3,2-b]pyridin-7-yl)-6-chloro-1a,2-dihydro-1H-cyclopropa[c]quinolin-3(7bH)-yl)-2-(((tetrahydro-2H-pyran-2-yl)oxy)methyl)pyrrolidine-1-carboxylate (1.0 g, 1.35 mmol) in dioxane (10 mL) was added zinc cyanide (560 mg, 4.77 mmol), N-ethyl-N-isopropylpropan-2-amine (524 mg, 4.05 mmol) and bis(tri-tert-butylphosphine) palladium(0) (138 mg, 0.27 mmol). After stirred at 90° C. for 12 h, the mixture was filtered. The filtrate was diluted with water (20 mL) and extracted with dichloromethane (15 mL×3). The combined organic layers were washed with brine (25 mL), dried over anhydrous sodium sulfate, filtered and the filtrate was concentrated under reduced pressure. The residue was purified by column chromatography to afford the title compound. LCMS R_{T}=1.998 min, m/z=731.4 [M+H]⁺.

### Step 6: (2R,4S)-tert-butyl 4-(6-cyano-4-(2-(hydroxymethyl)thieno[3,2-b]pyridin-7-yl)-1a,2-dihydro-1H-cyclopropa[c]quinolin-3(7bH)-yl)-2-(((tetrahydro-2H-pyran-2-yl)oxy)methyl)pyrrolidine-1-carboxylate

To a solution of (2R,4S)-tert-butyl 4-(4-(2-(((tert-butyldimethylsilyl)oxy)methyl)thieno[3,2-b]pyridin-7-yl)-6-cyano-1a,2-dihydro-1H-cyclopropa[c]quinolin-3(7bH)-yl)-2-(((tetrahydro-2H-pyran-2-yl)oxy)methyl)pyrrolidine-1-carboxylate (600 mg, 0.82 mmol) in THF (5 mL) were added tetrabutylammonium fluoride (0.8 mL, 1.0 M in THF). After stirred at 25° C. for 2 h, the mixture was diluted with water (20 mL) and extracted with ethyl acetate (15 mL×3). The combined organic layers were washed with brine (25 mL), dried over anhydrous sodium sulfate and concentrated under reduced pressure. The residue was purified by column chromatography to afford the title compound. LCMS R_{T}=0.929 min, m/z=617.3 [M+H]⁺.

### Step 7: (2R,4S)-tert-butyl 4-(6-cyano-4-(2-((6-oxo-4-(1-(trifluoromethyl)cyclopropyl)pyridazin-1(6H)-yl)methyl)thieno[3,2-b]pyridin-7-yl)-1a,2-dihydro-1H-cyclopropa[c]quinolin-3(7bH)-yl)-2-(((tetrahydro-2H-pyran-2-yl)oxy)methyl)pyrrolidine-1-carboxylate

To a solution of (2R,4S)-tert-butyl 4-(6-cyano-4-(2-(hydroxymethyl)thieno[3,2-b]pyridin-7-yl)-1a,2-dihydro-1H-cyclopropa[c]quinolin-3(7bH)-yl)-2-(((tetrahydro-2H-pyran-2-yl)oxy)methyl)pyrrolidine-1-carboxylate (160 mg, 0.26 mmol) in toluene (1.5 mL) was added 4-[1-(trifluoromethyl)cyclopropyl]-1H-pyridazin-6-one (79 mg, 0.39 mmol), tetramethylazodicarboxamide (89 mg, 0.52 mmol) and tributylphosphane (210 mg, 1.04 mmol). After stirred at 100° C. for 1 h, the mixture was concentrated under reduced pressure. The residue was purified by preparative TLC to afford the title compound. LCMS R_{T}=1.771 min, m/z=803.3 [M+H]⁺.

### Step 8: (2R,4S)-tert-butyl 4-((1aS,7bR)-6-cyano-4-(2-((6-oxo-4-(1-(trifluoromethyl)cyclopropyl)pyridazin-1(6H)-yl)methyl)thieno[3,2-b]pyridin-7-yl)-1a,2-dihydro-1H-cyclopropa[c]quinolin-3(7bH)-yl)-2-(hydroxymethyl)pyrrolidine-1-carboxylate and (2R,4S)-tert-butyl 4-((1aR,7bS)-6-cyano-4-(2-((6-oxo-4-(1-(trifluoromethyl)cyclopropyl)pyridazin-1(6H)-yl)methyl)thieno[3,2-b]pyridin-7-yl)-1a,2-dihydro-1H-cyclopropa[c]quinolin-3(7bH)-yl)-2-(hydroxymethyl)pyrrolidine-1-carboxylate

To a solution of (2R,4S)-tert-butyl 4-(6-cyano-4-(2-((6-oxo-4-(1-(trifluoromethyl)cyclopropyl)pyridazin-1(6H)-yl)methyl)thieno[3,2-b]pyridin-7-yl)-1a,2-dihydro-1H-cyclopropa[c]quinolin-3(7bH)-yl)-2-(((tetrahydro-2H-pyran-2-yl)oxy)methyl)pyrrolidine-1-carboxylate (200 mg, 0.25 mmol) in methanol (2 mL) was added 4-methylbenzenesulfonic acid hydrate (47 mg, 0.25 mmol). After stirred at 25° C. for 1 h, the mixture was concentrated under reduced pressure. The residue was purified by preparative TLC. The racemate was separated by SFC to afford (194.1, Rt=2.387 min, and (194.2, Rt=2.927 min, LCMS R_{T}=0.546 min, m/z=719.3 [M+H]⁺.)

### Step 9

To a solution of 194.1 (30 mg, 0.04 mmol) was dissolved in hydrochloric acid (1 mL, 4 M in dioxane). After stirred at 25° C. for 0.5 h, the mixture was concentrated under reduced pressure. The residue was purified by preparative HPLC to afford Example 194a. ¹H NMR (400 MHz, CD₃OD) δ=8.77 (s, 1H), 8.07 (s, 1H), 7.68 (d, J=1.6 Hz, 1H), 7.59 (s, 1H), 7.54-7.16 (m, 2H), 7.09 (d, J=2.0 Hz, 1H), 5.65 (s, 2H), 4.24-3.94 (m, 1H), 3.68-3.39 (m, 2H), 3.28 (s, 3H), 2.27-1.92 (m, 4H), 1.83-1.62 (m, 1H), 1.49 (s, 2H), 1.35-1.07 (m, 4H), 0.95 (s, 1H). LCMS R_{T}=0.460 min, m/z=619.2 [M+H]⁺.

To a solution of 194.2 (30 mg, 0.04 mmol) was dissolved in hydrochloric acid (1 mL, 4 M in dioxane). After stirred at 25° C. for 0.5 h, the mixture was concentrated under reduced pressure. The residue was purified by preparative HPLC to afford the title compound. ¹H NMR (400 MHz, CD₃OD) δ=8.82-8.65 (m, 1H), 8.48 (s, 1H), 8.05 (s, 1H), 7.65 (d, J=1.6 Hz, 1H), 7.59 (s, 1H), 7.53-7.30 (m, 2H), 7.07 (s, 1H), 5.70-5.54 (m, 2H), 3.90-3.63 (m, 1H), 3.48 (s, 2H), 3.30-3.17 (m, 3H), 3.11-2.79 (m, 3H), 2.19-2.04 (m, 2H), 1.51-1.45 (m, 2H), 1.25 (s, 2H), 1.20-1.04 (m, 2H), 0.87 (d, J=3.6 Hz, 1H). LCMS R_{T}=0.453 min, m/z=619.2 [M+H]⁺.

### Example 195a and 195b: (1aS,7bR)-3-((3S,5R)-5-(hydroxymethyl)pyrrolidin-3-yl)-4-(2-((4-(1-methylcyclopropyl)-6-oxopyridazin-1(6H)-yl)methyl)thieno[3,2-b]pyridin-7-yl)-1a,2,3,7b-tetrahydro-1H-cyclopropa[c]quinoline-6-carbonitrile, formic acid salt and (1aR,7bS)-3-((3S,5R)-5-(hydroxymethyl)pyrrolidin-3-yl)-4-(2-((4-(1-methylcyclopropyl)-6-oxopyridazin-1(6H)-yl)methyl)thieno[3,2-b]pyridin-7-yl)-1a,2,3,7b-tetrahydro-1H-cyclopropa[c]quinoline-6-carbonitrile, formic acid salt

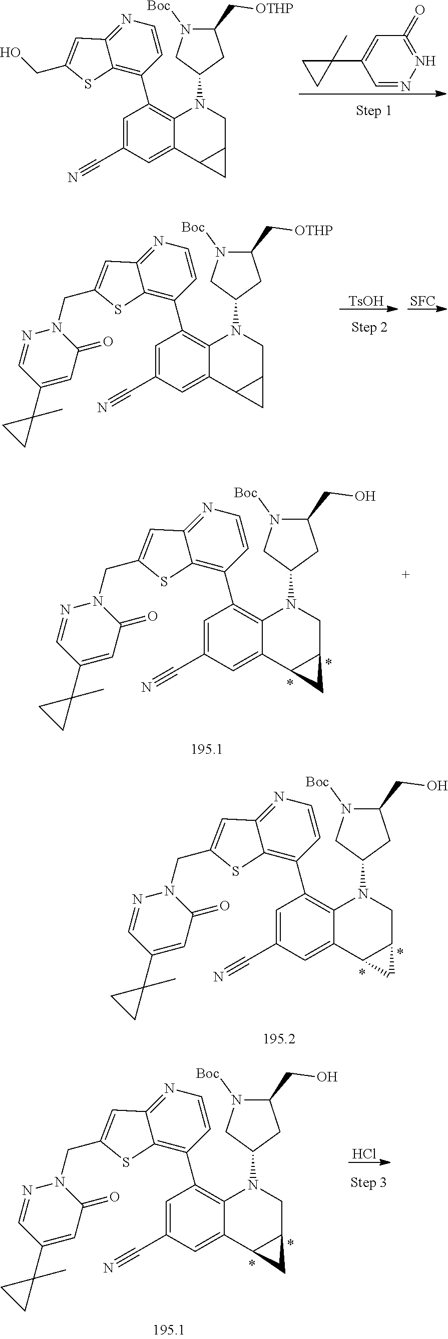

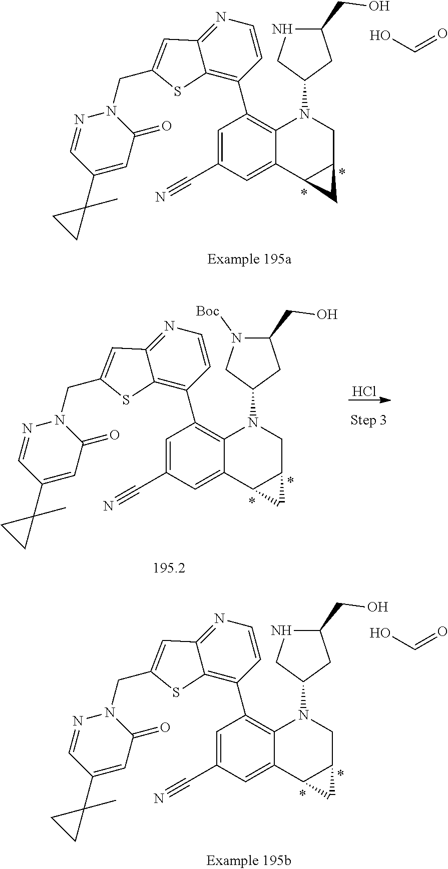

### Step 1: (2R,4S)-tert-butyl 4-(6-cyano-4-(2-((4-(1-methylcyclopropyl)-6-oxopyridazin-1(6H)-yl)methyl)thieno[3,2-b]pyridin-7-yl)-1a,2-dihydro-1H-cyclopropa[c]quinolin-3(7bH)-yl)-2-(((tetrahydro-2H-pyran-2-yl)oxy)methyl)pyrrolidine-1-carboxylate

To a solution of (2R,4S)-tert-butyl 4-(6-cyano-4-(2-(hydroxymethyl)thieno[3,2-b]pyridin-7-yl)-1a,2-dihydro-1H-cyclopropa[c]quinolin-3(7bH)-yl)-2-(((tetrahydro-2H-pyran-2-yl)oxy)methyl)pyrrolidine-1-carboxylate (130 mg, 0.21 mmol), 4-(1-methylcyclopropyl)-1H-pyridazin-6-one (47 mg, 0.32 mmol) and tetramethylazodicarboxamide (109 mg, 0.63 mmol) in toluene (2 mL) was added tributylphosphane (171 mg, 0.84 mmol). After stirred at 100° C. for 1 h, the mixture was concentrated under reduced pressure. The residue was purified by preparative TLC to give the title compound. LCMS R_{T}=0.594 min, m/z=749 [M+H]⁺.

### Step 2

To a solution of (2R,4S)-tert-butyl 4-(6-cyano-4-(2-((4-(1-methylcyclopropyl)-6-oxopyridazin-1(6H)-yl)methyl)thieno[3,2-b]pyridin-7-yl)-1a,2-dihydro-1H-cyclopropa[c]quinolin-3(7bH)-yl)-2-(((tetrahydro-2H-pyran-2-yl)oxy)methyl)pyrrolidine-1-carboxylate (130 mg, 0.17 mmol) in methanol (3 mL) was added hydrated p-toluenesulfonic acid (23 mg, 0.12 mmol). After stirred at 25° C. for 1 h, the reaction mixture was concentrated under reduced pressure to remove the solvent. The residue was purified by preparative TLC. The racemate was separated by SFC to give first eluting fraction (195.1, Rt=1.323 min, LCMS R_{T}=0.540 min, m/z=665.4 [M+H]⁺.) and second eluting fraction (195.2, Rt=2.498 min, LCMS R_{T}=0.540 min, m/z=665.4 [M+H]+).

### Step 3

The mixture of 195.1 (50 mg, 0.07 mmol) was dissolved in hydrochloric acid (1 mL, 4 M in dioxane). After stirred at 25° C. for 0.5 h, the reaction mixture was concentrated under reduced pressure. The residue was purified by preparative HPLC to give Example 195a. ¹H NMR (400 MHz, CD₃OD) δ=8.74 (s, 1H), 7.74 (d, J=2.0 Hz, 1H), 7.65 (d, J=2.0 Hz, 1H), 7.55 (s, 1H), 7.48-7.27 (m, 2H), 6.77 (d, J=2.4 Hz, 1H), 5.70-5.47 (m, 2H), 4.09 (d, J=6.4 Hz, 1H), 3.65-3.41 (m, 2H), 3.30-3.18 (m, 3H), 2.29-1.56 (m, 5H), 1.40 (s, 3H), 1.25-0.91 (m, 7H). LCMS R_{T}=1.430 min, m/z=565.4 [M+H]+

The mixture of 195.2 (50 mg, 0.07 mmol) was dissolved in hydrochloric acid (1 mL, 4 M in dioxane). After stirred at 25° C. for 0.5 h, the reaction mixture was concentrated under reduced pressure. The residue was purified by preparative HPLC to give Example 195b. ¹H NMR (400 MHz, CD₃OD) δ=8.74 (s, 1H), 7.74 (s, 1H), 7.64 (d, J=1.6 Hz, 1H), 7.55 (s, 1H), 7.51-7.31 (m, 2H), 6.77 (s, 1H), 5.59 (t, J=16.0 Hz, 2H), 3.93-3.74 (m, 1H), 3.31-3.16 (m, 5H), 3.08-2.81 (m, 3H), 2.12 (dd, J=3.6, 8.4 Hz, 2H), 1.40 (s, 3H), 1.19-0.84 (m, 7H). LCMS R_{T}=1.430 min, m/z=565.5 [M+H]⁺.

### Example 196: 1-[[7-[6-chloro-1-[(3R,5S)-5-(hydroxymethyl)-5-methyl-pyrrolidin-3-yl]-3,4-dihydro-2H-quinolin-8-yl]thieno[3,2-b]pyridin-2-yl]methyl]pyrrolidine-2,5-dione, formic acid salt

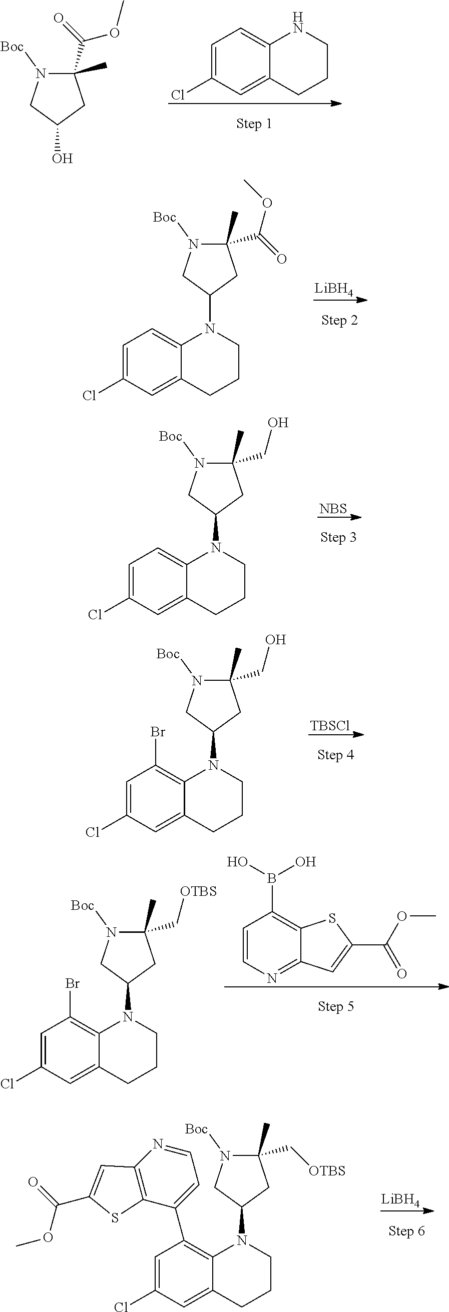

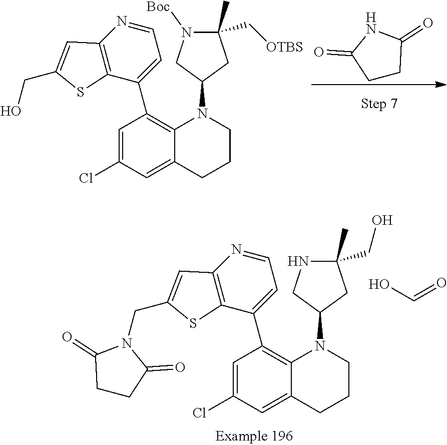

### Step 1: (2S)-1-tert-butyl 2-methyl 4-(6-chloro-3,4-dihydroquinolin-1(2H)-yl)-2-methylpyrrolidine-1,2-dicarboxylate

To a solution of (2S,4S)-1-tert-butyl 2-methyl 4-hydroxy-2-methylpyrrolidine-1,2-dicarboxylate (1.9 g, 7.33 mmol) in dichloromethane (20 mL) was added trifluoromethylanhydride (1.4 g, 10.99 mmol) and diisopropyl ethyl amine (2.5 g, 8.79 mmol) and 6-chloro-1,2,3,4-tetrahydroquinoline (1.2 g, 7.33 mmol) at −65° C. After stirred at 25° C. for 12 h, the reaction mixture was quenched by addition sodium bicarbonate (100 mL), and then extracted with dichloromethane (50 mL×3). The combined organic layers were dried over sodium sulphate, filtered and the filtrate was concentrated under reduced pressure to give a residue. The residue was purified by column chromatography to afford the title compound. LCMS R_{T}=2.833 min, m/z=409.1 [M+H]⁺.

### Step 2: (2S,4R)-tert-butyl 4-(6-chloro-3,4-dihydroquinolin-1(2H)-yl)-2-(hydroxymethyl)-2-methylpyrrolidine-1-carboxylate

To a solution of (2S)-1-tert-butyl 2-methyl 4-(6-chloro-3,4-dihydroquinolin-1(2H)-yl)-2-methylpyrrolidine-1,2-dicarboxylate (1.4 g, 3.42 mmol) in THF (20 mL) was added lithium borohydride (224 mg, 10.27 mmol) at 0° C. After stirred at 25° C. for 12 h, the reaction mixture was quenched by addition ammonium chloride (50 mL), and then extracted with ethyl acetate (50 mL×3). The combined organic layers were dried over sodium sulphate, filtered and the filtrate was concentrated under reduced pressure to give a residue. The residue was purified by column chromatography to afford the title compound. LCMS R_{T}=0.603 min, m/z=381.2 [M+H]⁺.

### Step 3: (2S,4R)-tert-butyl 4-(8-bromo-6-chloro-3,4-dihydroquinolin-1(2H)-yl)-2-(hydroxymethyl)-2-methylpyrrolidine-1-carboxylate

To a solution of (2S,4R)-tert-butyl 4-(6-chloro-3,4-dihydroquinolin-1(2H)-yl)-2-(hydroxymethyl)-2-methylpyrrolidine-1-carboxylate (200 mg, 0.53 mmol) in dichloromethane (5 mL) was added N-bromosuccinimide (84 mg, 0.47 mmol) at 0° C. After stirred at 25° C. for 3 h, the reaction mixture was quenched by addition of ammonium chloride (30 mL), and then extracted with dichloromethane (10 mL×3). The combined organic layers were dried over sodium sulphate, filtered and the filtrate was concentrated under reduced pressure to give a residue. The residue was purified by column chromatography to afford the title compound. LCMS R_{T}=2.217 min, m/z=461.2 [M+H]⁺.

### Step 4: (2S,4R)-tert-butyl 4-(8-bromo-6-chloro-3,4-dihydroquinolin-1(2H)-yl)-2-(((tert-butyldimethylsilyl)oxy)methyl)-2-methylpyrrolidine-1-carboxylate

To a solution of (2S,4R)-tert-butyl 4-(8-bromo-6-chloro-3,4-dihydroquinolin-1(2H)-yl)-2-(hydroxymethyl)-2-methylpyrrolidine-1-carboxylate (170 mg, 0.37 mmol) in dichloromethane (10 mL) was added imidazole (88 mg, 1.29 mmol) and tert-butyldimethylsilyl chloride (140 mg, 0.92 mmol). After stirred at 25° C. for 12 h, the reaction mixture was quenched by addition of ammonium chloride (20 mL), and then extracted with dichloromethane (10 mL×3). The combined organic layers were dried over sodium sulphate, filtered and the filtrate was concentrated under reduced pressure to give a residue. The residue was purified by column chromatography to afford the title compound. LCMS R_{T}=2.017 min, m/z=575.4 [M+H]⁺.

### Step 5: methyl 7-[1-[(3R,5S)-1-tert-butoxycarbonyl-5-[[tert-butyl(dimethyl)silyl]oxymethyl]-5-methyl-pyrrolidin-3-yl]-6-chloro-3,4-dihydro-2H-quinolin-8-yl]thieno[3,2-b]pyridine-2-carboxylate

To a solution of (2S,4R)-tert-butyl 4-(8-bromo-6-chloro-3,4-dihydroquinolin-1(2H)-yl)-2-(((tert-butyldimethylsilyl)oxy)methyl)-2-methylpyrrolidine-1-carboxylate (140 mg, 0.24 mmol) in dioxane (4 mL) and water (2 mL) was added (2-methoxycarbonylthieno[3,2-b]pyridin-7-yl)boronic acid (88 mg, 0.37 mmol) and dichloro[1,1′-bis(diphenylphosphino)ferrocene]palladium(II) (36 mg, 0.05 mmol) and cesium carbonate (159 mg, 0.49 mmol) under nitrogen atmosphere. After stirred at 120° C. for 1 h, the reaction mixture was diluted with water (10 mL), extracted with ethyl acetate (10 mL×2). The combined organic layers were washed with brine (10 mL), dried over anhydrous sodium sulfate and concentrated under reduced pressure. The residue was purified by column chromatography to afford the title compound. LCMS R_{T}=3.631 min, m/z=686.4 [M+H]⁺.

### Step 6: (2S,4R)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)-2-methylpyrrolidine-1-carboxylate

To a solution of methyl 7-[1-[(3R,5S)-1-tert-butoxycarbonyl-5-[[tert-butyl(dimethyl)silyl]oxymethyl]-5-methyl-pyrrolidin-3-yl]-6-chloro-3,4-dihydro-2H-quinolin-8-yl]thieno[3,2-b]pyridine-2-carboxylate (180 mg, 0.26 mmol) in THF (5 mL) was added lithium borohydride (17 mg, 0.79 mmol) at 0° C. After stirred at 25° C. for 12 h, the reaction mixture was quenched by addition of ammonium chloride (10 mL), and then extracted with ethyl acetate (10 mL×3). The combined organic layers were dried over sodium sulphate, filtered and the filtrate was concentrated under reduced pressure to give a residue. The residue was purified by column chromatography to afford the title compound. LCMS R_{T}=1.951 min, m/z=658.2 [M+H]⁺.

### Step 7

Example 196 was prepared from (2S,4R)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)-2-methylpyrrolidine-1-carboxylate and succinimide, following the procedure described in the synthesis of Example 108. ¹H NMR (400 MHz, CD₃OD) δ=8.69 (d, J=4.8 Hz, 1H), 7.48 (s, 1H), 7.42 (d, J=4.8 Hz, 1H), 7.15 (dd, J=2.4, 14.4 Hz, 2H), 4.95 (s, 2H), 3.76 (s, 1H), 3.28-3.01 (m, 4H), 2.92-2.73 (m, 8H), 2.18-1.76 (m, 4H), 1.22 (s, 3H). LCMS R_{T}=1.462 min, m/z=525.1 [M+H]⁺.

### Example 197: 1-((7-(6-chloro-1-((3S,5S)-5-(hydroxymethyl)-5-methylpyrrolidin-3-yl)-1,2,3,4-tetrahydroquinolin-8-yl)thieno[3,2-b]pyridin-2-yl)methyl)pyrrolidine-2,5-dione, formic acid salt

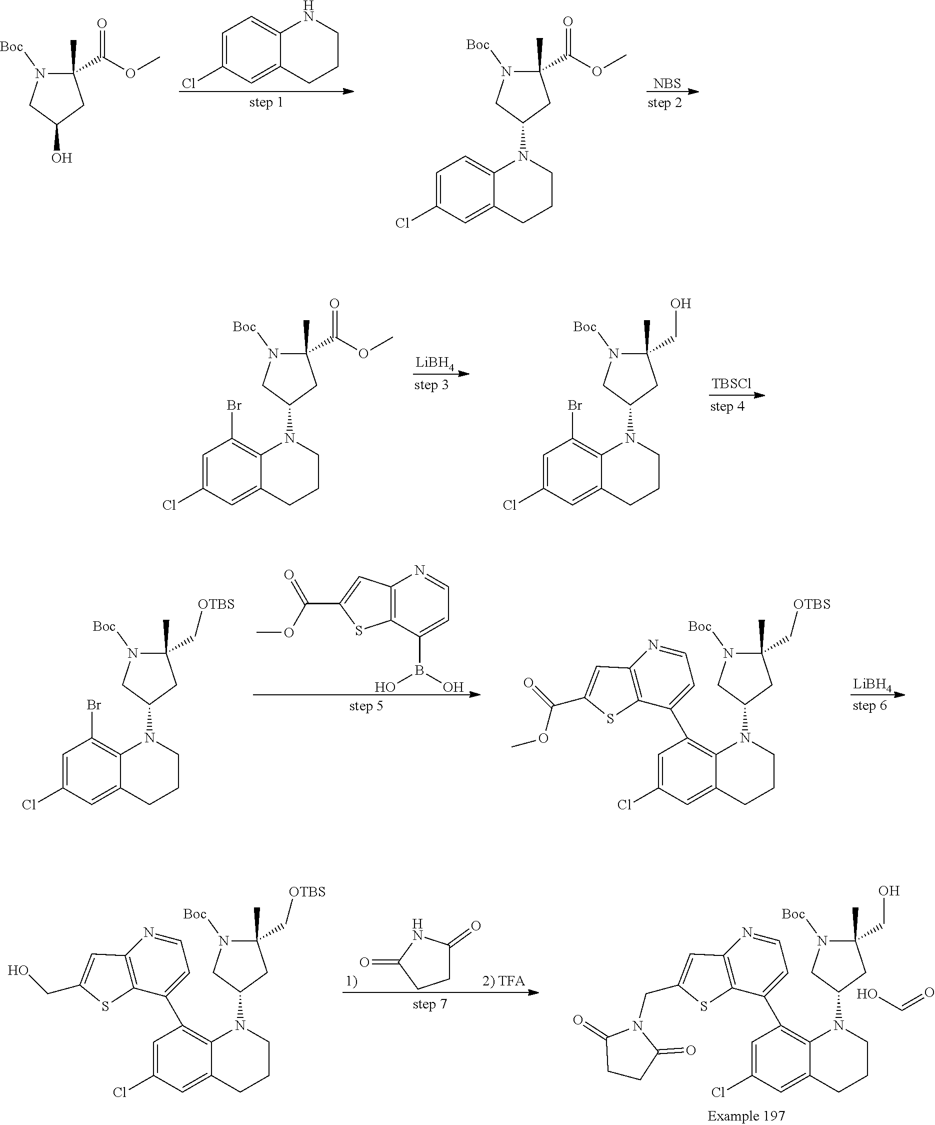

### Step 1: (2S,4S)-1-tert-butyl 2-methyl 4-(6-chloro-3,4-dihydroquinolin-1(2H)-yl)-2-methylpyrrolidine-1,2-dicarboxylate

To a solution of (2S,4R)-4-hydroxy-2-methyl-pyrrolidine-1,2-dicarboxylate (1.2 g, 4.51 mmol) in dichloromethane (10 mL) was added diisopropylethylamine (875 mg, 6.77 mmol), trifluoromethylsulfonyl trifluoromethanesulfonate (1.5 g, 5.41 mmol) at −65° C. The mixture was allowed to warm to −30° C. and then added a solution of 6-chloro-1,2,3,4-tetrahydroquinoline (890 mg, 4.51 mmol). After stirred at 20° C. for 3 h, the reaction mixture was quenched by addition of water (100 mL) and extracted with dichloromethane (200 mL). The combined organic layers were washed with brine (200 mL), dried over anhydrous sodium sulfate, filtered and the filtrate was concentrated under reduced pressure. The residue was purified by column chromatography to afford the title compound. LCMS R_{T}=1.693 min, m/z=409.3 [M+H]⁺.

### Step 2: (2S,4S)-1-tert-butyl 2-methyl 4-(8-bromo-6-chloro-3,4-dihydroquinolin-1(2H)-yl)-2-methylpyrrolidine-1,2-dicarboxylate

To a solution of (2S,4S)-1-tert-butyl 2-methyl 4-(6-chloro-3,4-dihydroquinolin-1(2H)-yl)-2-methylpyrrolidine-1,2-dicarboxylate (425 mg, 1.04 mmol) in dimethyl formamide (1 mL) was added N-bromosuccinimide (185 mg, 1.04 mmol) at 0° C. After stirred at 20° C. for 1 h, the residue was purified by column chromatography to afford the title compound. LCMS R_{T}=1.947 min, m/z=489.2 [M+H]⁺.

### Step 3: (2S,4S)-tert-butyl 4-(8-bromo-6-chloro-3,4-dihydroquinolin-1(2H)-yl)-2-(hydroxymethyl)-2-methylpyrrolidine-1-carboxylate

To a solution of (2S,4S)-1-tert-butyl 2-methyl 4-(8-bromo-6-chloro-3,4-dihydroquinolin-1(2H)-yl)-2-methylpyrrolidine-1,2-dicarboxylate (280 mg, 0.574 mmol) in DMF (2 mL) was added lithium borohydride (38 mg, 1.72 mmol) at 0° C. under nitrogen atmosphere. After stirred at 20° C. for 12 h, the reaction mixture was quenched by addition of water (50 mL) and extracted with ethyl acetate (100 mL). The combined organic layers were washed with brine (100 mL), dried over anhydrous sodium sulfate, filtered and the filtrate was concentrated under reduced pressure. The residue was purified by column chromatography to afford the title compound. LCMS R_{T}=1.782 min, m/z=461.2 [M+H]⁺.

### Step 4: (2S,4S)-tert-butyl 4-(8-bromo-6-chloro-3,4-dihydroquinolin-1(2H)-yl)-2-(((tert-butyldimethylsilyl)oxy)methyl)-2-methylpyrrolidine-1-carboxylate

To a solution of (2S,4S)-tert-butyl 4-(8-bromo-6-chloro-3,4-dihydroquinolin-1(2H)-yl)-2-(hydroxymethyl)-2-methylpyrrolidine-1-carboxylate (215 mg, 0.47 mmol) in dichloromethane (3 mL) was added tert-butyl-chloro-dimethyl-silane (176 mg, 1 mmol) and imidazole (127 mg, 1.87 mmol) at 0° C. After stirred at 30° C. for 16 h, the reaction mixture was quenched by addition of water (400 mL) and extracted with dichloromethane (600 mL). The combined organic layers were washed with brine (500 mL), dried over anhydrous sodium sulfate, filtered and the filtrate was concentrated under reduced pressure. The residue was purified by column chromatography to afford the title compound. LCMS R_{T}=2.697 min, m/z=575.1 [M+H]⁺.

### Step 5: methyl 7-(1-((3S,5S)-1-(tert-butoxycarbonyl)-5-(((tert-butyldimethylsilyl)oxy)methyl)-5-methylpyrrolidin-3-yl)-6-chloro-1,2,3,4-tetrahydroquinolin-8-yl)thieno[3,2-b]pyridine-2-carboxylate

To a solution of (2S,4S)-tert-butyl 4-(8-bromo-6-chloro-3,4-dihydroquinolin-1(2H)-yl)-2-(((tert-butyldimethylsilyl)oxy)methyl)-2-methylpyrrolidine-1-carboxylate (180 mg, 0.31 mmol) in dioxane (5 mL) and water (0.5 mL) was added [2-[[tert-butyl(dimethyl)silyl]oxymethyl]thieno[3,2-b]pyridin-7-yl]boronic acid (111 mg, 0.47 mmol), potassium carbonate (204 mg, 0.63 mmol), [1,1′-bis(diphenylphosphino)ferrocene]dichloropalladium(II) (46 mg, 0.063 mmol). After stirred at 100° C. for 3 h under nitrogen atmosphere, the reaction mixture was filtered and the filtrate was concentrated under reduced pressure. The residue was purified by column chromatography to afford the title compound. LCMS R_{T}=2.408 min, m/z=686.2 [M+H]⁺.

### Step 6: (2S,4S)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)-2-methylpyrrolidine-1-carboxylate

To a solution of methyl 7-(1-((3S,5S)-1-(tert-butoxycarbonyl)-5-(((tert-butyldimethylsilyl)oxy)methyl)-5-methylpyrrolidin-3-yl)-6-chloro-1,2,3,4-tetrahydroquinolin-8-yl)thieno[3,2-b]pyridine-2-carboxylate (180 mg, 0.26 mmol) in DMF (3 mL) was added lithium borohydride (17 mg, 0.79 mmol) at 0° C. After stirred at 20° C. for 12 h, the reaction mixture was quenched by addition of water (50 mL) and extracted with ethyl acetate (100 mL). The combined organic layers were washed with brine (100 mL), dried over anhydrous sodium sulfate, filtered and the filtrate was concentrated under reduced pressure. The residue was purified by column chromatography to afford the title compound. LCMS R_{T}=2.260 min, m/z=658.5 [M+H]⁺.

### Step 7

Example 197 was prepared from (2S,4S)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)-2-methylpyrrolidine-1-carboxylate and succinimide, following the procedure described in the synthesis of Example 108. ¹H NMR (400 MHz, CD₃OD) δ=8.51-8.49 (m, 1H), 8.29-8.26 (m, 1H), 7.29-7.21 (m, 2H), 6.98-6.90 (m, 2H), 4.74-4.70 (m, 2H), 3.61-3.46 (m, 1H), 3.22-3.12 (m, 2H), 3.05-2.95 (m, 2H), 2.53 (s, 8H), 1.96-1.39 (m, 4H), 0.61-0.37 (m, 3H) LCMS R_{T}=1.113 min, m/z=525.4 [M+H]⁺.

### Example 198: 1-((7-(6-chloro-1-((3S,5R)-5-(hydroxymethyl)-5-methylpyrrolidin-3-yl)-1,2,3,4-tetrahydroquinolin-8-yl)thieno[3,2-b]pyridin-2-yl)methyl)pyrrolidine-2,5-dione, formic acid salt

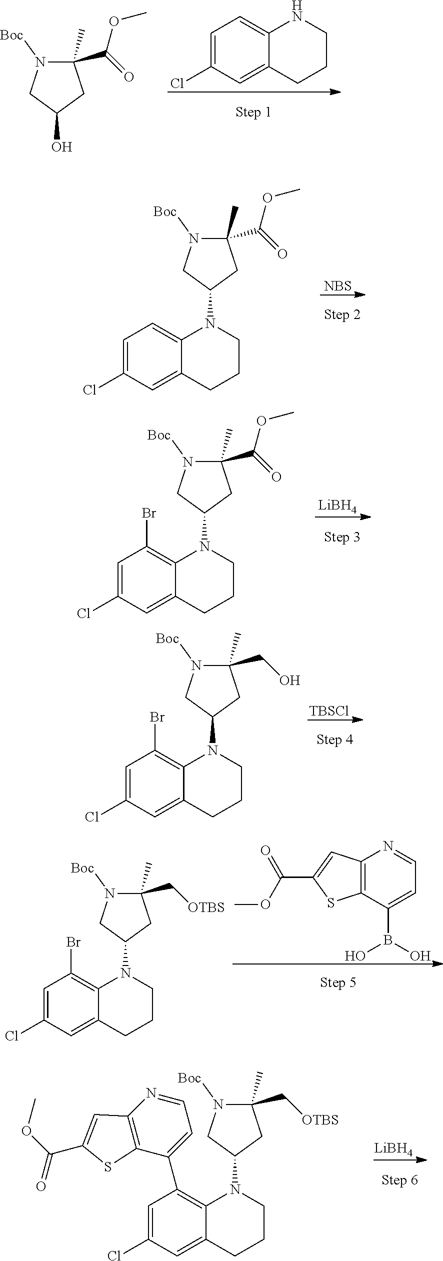

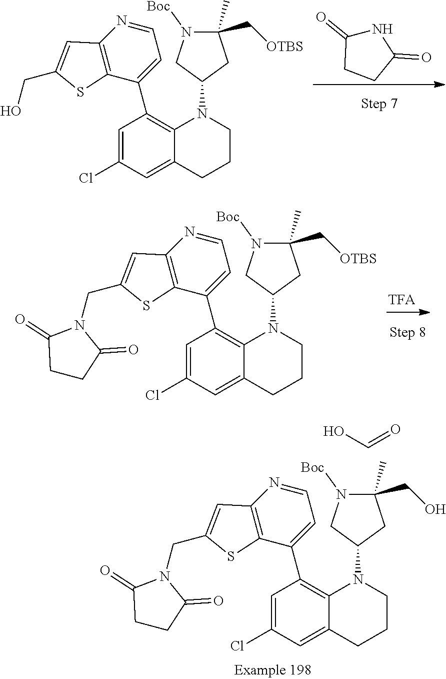

### Step 1: (2R,4S)-1-tert-butyl 2-methyl 4-(6-chloro-3,4-dihydroquinolin-1(2H)-yl)-2-methylpyrrolidine-1,2-dicarboxylate

To a solution of (2R,4R)-1-tert-butyl 2-methyl 4-hydroxy-2-methylpyrrolidine-1,2-dicarboxylate (2.0 g, 7.71 mmol) in dichloromethane (35 mL) was added N-ethyl-N-isopropylpropan-2-amine (1.5 g, 11.57 mmol), trifluoromethanesulfonic anhydride (2.6 g, 9.26 mmol) at −65° C. After stirred at −30° C. for 1 h, 6-chloro-1,2,3,4-tetrahydroquinoline (2.3 g, 11.57 mmol) was added at −65° C. After stirred at 35° C. for 15 h, the mixture was quenched by addition of saturated aqueous sodium hydrogencarbonate (100 mL) and extracted with dichloromethane (50 mL×2). The combined organic layers were washed with brine (100 mL), dried over anhydrous sodium sulfate and concentrated under reduced pressure. The residue was purified by column chromatography to afford the title compound. ¹H NMR (400 MHz, CDCl₃) δ=7.07-6.92 (m, 2H), 6.70-6.59 (m, 1H), 4.53-4.38 (m, 1H), 3.76 (s, 4H), 3.56-3.40 (m, 1H), 3.27-3.17 (m, 2H), 2.71 (t, J=6.0 Hz, 2H), 2.45-2.35 (m, 1H), 2.11-1.99 (m, 1H), 1.91-1.86 (m, 2H), 1.60 (s, 3H), 1.45-1.41 (m, 9H). LCMS R_{T}=0.648 min, m/z=409.1 [M+H]⁺.

### Step 2: (2R,4S)-1-tert-butyl 2-methyl 4-(8-bromo-6-chloro-3,4-dihydroquinolin-1(2H)-yl)-2-methylpyrrolidine-1,2-dicarboxylate

To a solution of (2R,4S)-1-tert-butyl 2-methyl 4-(6-chloro-3,4-dihydroquinolin-1(2H)-yl)-2-methylpyrrolidine-1,2-dicarboxylate (1.0 g, 2.45 mmol) in DMF (15 mL) was added 1-bromopyrrolidine-2,5-dione (435 mg, 2.45 mmol). After stirred at 10° C. for 1 h, the mixture was diluted with ethyl acetate (50 mL) and washed with water (80 mL×2). The organic layer was dried over anhydrous sodium sulfate and concentrated under reduced pressure. The residue was purified by column chromatography to afford the title compound. LCMS R_{T}=0.665 min, m/z=489.1 [M+H]⁺.

### Step 3: (2R,4S)-tert-butyl 4-(8-bromo-6-chloro-3,4-dihydroquinolin-1(2H)-yl)-2-(hydroxymethyl)-2-methylpyrrolidine-1-carboxylate

To a solution of (2R,4S)-1-tert-butyl 2-methyl 4-(8-bromo-6-chloro-3,4-dihydroquinolin-1(2H)-yl)-2-methylpyrrolidine-1,2-dicarboxylate (600 mg, 1.23 mmol) in THF (8 mL) was added lithium borohydride (110 mg, 5.05 mmol) at −65° C. After stirred at 35° C. for 12 h, the mixture was quenched by addition of saturated aqueous ammonium chloride (100 mL) and extracted with ethyl acetate (40 mL×2). The combined organic layers were washed with brine (50 mL), dried over anhydrous sodium sulfate and concentrated under reduced pressure. The residue was purified by column chromatography to afford the title compound. ¹H NMR (400 MHz, CDCl₃) δ=7.39 (s, 1H), 7.01-6.92 (m, 1H), 4.41-4.13 (m, 2H), 3.83-3.67 (m, 4H), 3.63-3.51 (m, 1H), 3.41-3.29 (m, 1H), 3.19-3.04 (m, 2H), 2.75 (t, J=6.4 Hz, 2H), 1.97-1.73 (m, 5H), 1.50-1.45 (m, 9H). LCMS R_{T}=0.648 min, m/z=460.8 [M+H]⁺.

### Step 4: (2R,4S)-tert-butyl 4-(8-bromo-6-chloro-3,4-dihydroquinolin-1(2H)-yl)-2-(((tert-butyldimethylsilyl)oxy)methyl)-2-methylpyrrolidine-1-carboxylate

To a solution of (2R,4S)-tert-butyl 4-(8-bromo-6-chloro-3,4-dihydroquinolin-1(2H)-yl)-2-(hydroxymethyl)-2-methylpyrrolidine-1-carboxylate (450 mg, 0.98 mmol) in dichloromethane (15 mL) was added 1H-imidazole (167 mg, 2.45 mmol) and tert-butylchlorodimethylsilane (295 mg, 1.96 mmol). After stirred at 20° C. for 2 h, the mixture was diluted with dichloromethane (20 mL). The mixture was washed with brine (15 mL), dried over anhydrous sodium sulfate and concentrated under reduced pressure. The residue was purified by column chromatography to afford the title compound. LCMS R_{T}=4.983 min, m/z=575.2 [M+H]⁺.

### Step 5: methyl 7-(1-((3S,5R)-1-(tert-butoxycarbonyl)-5-(((tert-butyldimethylsilyl)oxy)methyl)-5-methylpyrrolidin-3-yl)-6-chloro-1,2,3,4-tetrahydroquinolin-8-yl)thieno[3,2-b]pyridine-2-carboxylate

To a solution of (2R,4S)-tert-butyl 4-(8-bromo-6-chloro-3,4-dihydroquinolin-1(2H)-yl)-2-(((tert-butyldimethylsilyl)oxy)methyl)-2-methylpyrrolidine-1-carboxylate (450 mg, 0.78 mmol) and (2-(methoxycarbonyl)thieno[3,2-b]pyridin-7-yl)boronic acid (260 mg, 1.10 mmol) in water (0.8 mL) and dioxane (10 mL) was added dichloro[1,1′-bis(diphenylphosphino)ferrocene]palladium(II) (115 mg, 0.16 mmol) and potassium carbonate (217 mg, 1.57 mmol). After stirred at 90° C. for 2 h under nitrogen atmosphere, the mixture was concentrated under reduced pressure. The residue was purified by column chromatography to afford the title compound. LCMS R_{T}=2.817 min, m/z=686.5 [M+H]⁺.

### Step 6: (2R,4S)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)-2-methylpyrrolidine-1-carboxylate

To a solution of methyl 7-(1-((3S,5R)-1-(tert-butoxycarbonyl)-5-(((tert-butyldimethylsilyl)oxy)methyl)-5-methylpyrrolidin-3-yl)-6-chloro-1,2,3,4-tetrahydroquinolin-8-yl)thieno[3,2-b]pyridine-2-carboxylate (170 mg, 0.25 mmol) in THF (2 mL) was added lithium borohydride (16 mg, 0.74 mmol). After stirred at room temperature for 12 h, the mixture was quenched by addition of saturated aqueous ammonium chloride (30 mL) and extracted with ethyl acetate (10 mL×2). The combined organic layers were washed with brine (15 mL), dried over anhydrous sodium sulfate and concentrated under reduced pressure. The residue was purified by preparative HPLC to afford the title compound. LCMS R_{T}=2.227 min, m/z=658.5 [M+H]⁺.

### Step 7: (2R,4S)-tert-butyl 2-(((tert-butyldimethylsilyl)oxy)methyl)-4-(6-chloro-8-(2-((2,5-dioxopyrrolidin-1-yl)methyl)thieno[3,2-b]pyridin-7-yl)-3,4-dihydroquinolin-1(2H)-yl)-2-methylpyrrolidine-1-carboxylate

To a solution of (2R,4S)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)-2-methylpyrrolidine-1-carboxylate (70 mg, 0.11 mmol) in toluene (1.5 mL) was added succinimide (21 mg, 0.21 mmol), tetramethylazodicarboxamide (55 mg, 0.32 mmol) and tributylphosphine (108 mg, 0.53 mmol). After stirred at 95° C. for 1 h, the mixture was concentrated under reduced pressure. The residue was purified by preparative TLC to afford the title compound. LCMS R_{T}=1.197 min, m/z=739.1 [M+H]⁺.

### Step 8

To a solution of (2R,4S)-tert-butyl 2-(((tert-butyldimethylsilyl)oxy)methyl)-4-(6-chloro-8-(2-((2,5-dioxopyrrolidin-1-yl)methyl)thieno[3,2-b]pyridin-7-yl)-3,4-dihydroquinolin-1(2H)-yl)-2-methylpyrrolidine-1-carboxylate (45 mg, 0.061 mmol) in dichloromethane (1.5 mL) was added trifluoroacetic acid (693 mg, 6.09 mmol). After stirred at 20° C. for 2 h. The mixture was concentrated under reduced pressure. The residue was purified by preparative HPLC to afford Example 198. ¹H NMR (400 MHz, CD₃OD) δ=8.69 (d, J=4.8 Hz, 1H), 8.51 (s, 1H), 7.48 (s, 1H), 7.42 (d, J=4.8 Hz, 1H), 7.15 (dd, J=2.4, 14.4 Hz, 2H), 4.95 (s, 2H), 3.77 (s, 1H), 3.26-2.67 (m, 12H), 2.06-1.46 (m, 4H), 1.22 (s, 3H). LCMS R_{T}=1.073 min, m/z=525.3 [M+H]⁺.

### Example 199: 1-((7-(6-chloro-1-((3R,5R)-5-(hydroxymethyl)-5-methylpyrrolidin-3-yl)-1,2,3,4-tetrahydroquinolin-8-yl)thieno[3,2-b]pyridin-2-yl)methyl)pyrrolidine-2,5-dione, formic acid salt

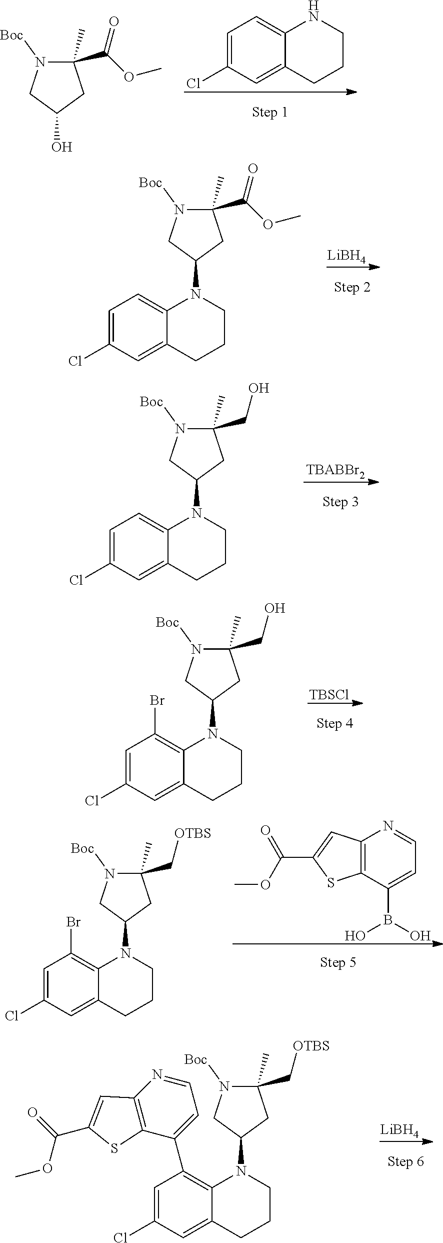

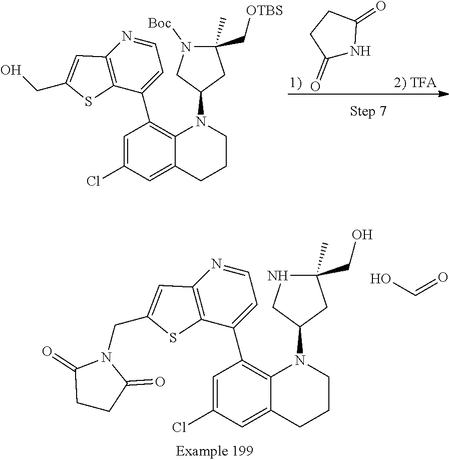

### Step 1: (2R,4R)-1-tert-butyl 2-methyl 4-(6-chloro-3,4-dihydroquinolin-1(2H)-yl)-2-methylpyrrolidine-1,2-dicarboxylate

To a solution of (2R,4S)-1-tert-butyl 2-methyl 4-hydroxy-2-methylpyrrolidine-1,2-dicarboxylate (3.9 g, 15.04 mmol) in dichloromethane (45 mL) were added N-ethyl-N-isopropylpropan-2-amine (2.9 g, 22.56 mmol) and trifluoromethanesulfonic anhydride (5.1 g, 18.05 mmol) at −65° C. The mixture was warmed to −30° C. and was added 6-chloro-1,2,3,4-tetrahydroquinoline (2.5 g, 15.04 mmol). After stirred at 25° C. for 2 h, the reaction mixture was diluted with aqueous sodium bicarbonate solution (70 mL) and extracted with ethyl acetate (50 mL×3). The combined organic layers were washed with brine (50 mL), dried over anhydrous sodium sulfate and concentrated under reduced pressure. The residue was purified by column chromatography to give the title compound. LCMS R_{T}=2.662 min, m/z=409.1 [M+H]⁺.

### Step 2: (2R,4R)-tert-butyl 4-(6-chloro-3,4-dihydroquinolin-1(2H)-yl)-2-(hydroxymethyl)-2-methylpyrrolidine-1-carboxylate

To a solution of (2R,4R)-1-tert-butyl 2-methyl 4-(6-chloro-3,4-dihydroquinolin-1(2H)-yl)-2-methylpyrrolidine-1,2-dicarboxylate (3.0 g, 7.34 mmol) in THF (40 mL) was added lithium borohydride (479 mg, 22.01 mmol). After stirred at −0° C. for 12 h, the reaction mixture was diluted with saturated ammonium chloride (20 mL), extracted with ethyl acetate (15 mL×3). The combined organic layers were washed with brine (25 mL), dried over anhydrous sodium sulfate and concentrated under reduced pressure. The residue was purified by column chromatography to give the title compound. LCMS R_{T}=1.077 min, m/z=381.1 [M+H]⁺

### Step 3: (2R,4R)-tert-butyl 4-(8-bromo-6-chloro-3,4-dihydroquinolin-1(2H)-yl)-2-(hydroxymethyl)-2-methylpyrrolidine-1-carboxylate

To a solution of (2R,4R)-tert-butyl 4-(6-chloro-3,4-dihydroquinolin-1(2H)-yl)-2-(hydroxymethyl)-2-methylpyrrolidine-1-carboxylate (380 mg, 0.1 mmol) in dichloromethane (5 mL) and methanol (5 mL) was added tetrabutylammonium tribromide (481 mg, 0.1 mmol). After stirred at 25° C. for 0.5 h, the reaction mixture was diluted with water (10 mL), extracted with ethyl acetate (5 mL×3). The combined organic layers were washed with brine (10 mL), dried over anhydrous sodium sulfate and concentrated under reduced pressure. The residue was purified by column chromatography to give the title compound. LCMS R_{T}=2.247 min, m/z=461.2 [M+H]⁺.

### Step 4: (2R,4R)-tert-butyl 4-(8-bromo-6-chloro-3,4-dihydroquinolin-1(2H)-yl)-2-(((tert-butyldimethylsilyl)oxy)methyl)-2-methylpyrrolidine-1-carboxylate

To a solution of (2R,4R)-tert-butyl 4-(8-bromo-6-chloro-3,4-dihydroquinolin-1(2H)-yl)-2-(hydroxymethyl)-2-methylpyrrolidine-1-carboxylate (280 mg, 0.61 mmol) in dichloromethane (10 mL) were added tert-butyldimethylsilyl chloride (229 mg, 1.52 mmol) and imidazole (166 mg, 2.44 mmol). After stirred at 20° C. for 1 h, the residue was diluted with water (20 mL), extracted with ethyl acetate (10 mL×3). The combined organic layers were washed with brine (20 mL), dried over anhydrous sodium sulfate and concentrated under reduced pressure. The residue was purified by column chromatography to give the title compound.

### Step 5: methyl 7-(1-((3R,5R)-1-(tert-butoxycarbonyl)-5-(((tert-butyldimethylsilyl)oxy)methyl)-5-methylpyrrolidin-3-yl)-6-chloro-1,2,3,4-tetrahydroquinolin-8-yl)thieno[3,2-b]pyridine-2-carboxylate

To a solution of (2R,4R)-tert-butyl 4-(8-bromo-6-chloro-3,4-dihydroquinolin-1(2H)-yl)-2-(((tert-butyldimethylsilyl)oxy)methyl)-2-methylpyrrolidine-1-carboxylate (290 mg, 0.51 mmol) in dioxane (10 mL) and water (1 mL) were added potassium carbonate (140 mg, 1.01 mmol), (2-methoxycarbonylthieno[3,2-b]pyridin-7-yl)boronic acid (180 mg 0.76 mmol) and dichloro[1,1′-bis(diphenylphosphino)ferrocene]palladium(II) (74 mg, 0.1 mmol). After stirred at 95° C. for 3 h, the reaction mixture was filtered. Then the filtrate was concentrated under reduced pressure. The residue was purified by column chromatography to afford the title compound. LCMS R_{T}=2.503 min, m/z=686.2 [M+H]⁺.

### Step 6: (2R,4R)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)-2-methylpyrrolidine-1-carboxylate

To a solution of methyl 7-[1-[(3R,5R)-1-tert-butoxycarbonyl-5-[[tert-butyl(dimethyl)silyl]oxymethyl]-5-methyl-pyrrolidin-3-yl]-6-chloro-3,4-dihydro-2H-quinolin-8-yl]thieno[3,2-b]pyridine-2-carboxylate (250 mg, 0.36 mmol) in THF (8 mL) was added lithium borohydride (24 mg, 1.09 mmol) at 0° C. After stirred at 20° C. for 1 h, the reaction mixture was diluted with saturated ammonium chloride (10 mL), extracted with ethyl acetate (8 mL×3). The combined organic layers were washed with brine (15 mL), dried over anhydrous sodium sulfate and concentrated under reduced pressure. The residue was purified by column chromatography to afford the title compound. LCMS R_{T}=2.308 min, m/z=658.3 [M+H]⁺

### Step 7

To a solution of (2R,4R)-tert-butyl 2-(((tert-butyldimethylsilyl)oxy)methyl)-4-(6-chloro-8-(2-(hydroxymethyl)thieno[3,2-b]pyridin-7-yl)-3,4-dihydroquinolin-1(2H)-yl)-2-methylpyrrolidine-1-carboxylate (140 mg, 0.21 mmol) in toluene (1.5 mL) was added succinimide (63 mg, 0.64 mmol), (3E)-3-(dimethylcarbamoylimino)-1,1-dimethyl-urea (110 mg, 0.64 mmol) and tributylphosphane (258 mg, 1.28 mmol) at 25° C. After stirred at 90° C. for 1 h, the reaction mixture was diluted with saturated ammonium chloride (10 mL), extracted with ethyl acetate (5 mL×3). The combined organic layers were washed with brine (10 mL), dried over anhydrous sodium sulfate and concentrated under reduced pressure. The residue was dissolved in trifluoroacetic acid (770 mg, 6.75 mmol). After stirred at 25° C. for 2 h, the reaction mixture was concentrated under reduced pressure. The residue was purified by preparative HPLC to give Example 199. ¹H NMR (400 MHz, CD₃OD) δ=8.75-8.68 (m, 1H), 8.56-8.41 (m, 1H), 7.51-7.45 (m, 2H), 7.20-7.13 (m, 2H), 4.94 (s, 2H), 3.84-3.68 (m, 1H), 3.48-3.35 (m, 2H), 3.28-3.17 (m, 2H), 2.79-2.73 (m, 1H), 3.08-2.53 (m, 7H), 2.11-1.56 (m, 4H), 0.83-0.63 (m, 3H). LCMS R_{T}=1.130 min, m/z=525.3 [M+H]⁺

### Example 200: 1-((7-(6-chloro-1-((3S,5R)-5-(methoxymethyl)-5-methylpyrrolidin-3-yl)-1,2,3,4-tetrahydroquinolin-8-yl)thieno[3,2-b]pyridin-2-yl)methyl)pyrrolidine-2,5-dione, formic acid salt

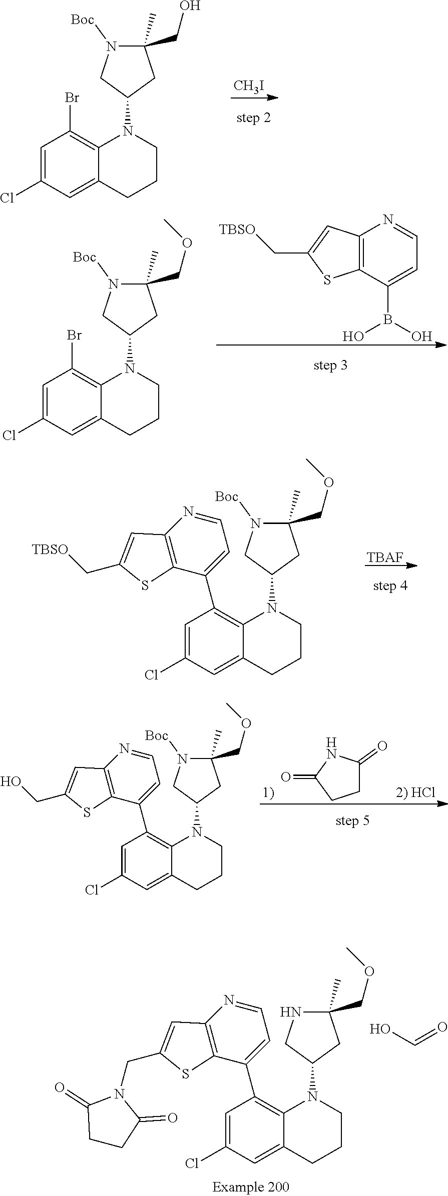

### Step 1: (2R,4S)-tert-butyl 4-(8-bromo-6-chloro-3,4-dihydroquinolin-1(2H)-yl)-2-(hydroxymethyl)-2-methylpyrrolidine-1-carboxylate

To a solution of (2R,4S)-1-tert-butyl 2-methyl 4-(8-bromo-6-chloro-3,4-dihydroquinolin-1(2H)-yl)-2-methylpyrrolidine-1,2-dicarboxylate (300 mg, 0.615 mmol) in THF (5 mL) was added lithium borohydride (40 mg, 1.84 mmol) at 0° C. After the mixture was stirred at 20° C. for 2 h, the reaction mixture was quenched with saturated aqueous ammonium chloride (20 mL) and extracted with dichloromethane (10 mL×2). The combined organic layers were washed with brine (20 mL), dried over anhydrous sodium sulfate, filtered and the filtrate was concentrated under reduced pressure. The residue was purified by column chromatography to afford the title compound. LCMS R_{T}=1.887 min, m/z=459.1 [M+H]⁺

### Step 2: (2R,4S)-tert-butyl 4-(8-bromo-6-chloro-3,4-dihydroquinolin-1(2H)-yl)-2-(methoxymethyl)-2-methylpyrrolidine-1-carboxylate

To a solution of (2R,4S)-tert-butyl 4-(8-bromo-6-chloro-3,4-dihydroquinolin-1(2H)-yl)-2-(hydroxymethyl)-2-methylpyrrolidine-1-carboxylate (170 mg, 0.37 mmol) in THF (2 mL) was added lithium bis(trimethylsilyl)amide (1 M, 0.56 mL) at 0° C. After stirred for 30 min, iodomethane (262 mg, 1.85 mmol) was added. After stirred at 20° C. for 2 h, the reaction mixture was quenched with saturated aqueous ammonium chloride (10 mL) and extracted with dichloromethane (5 mL×2). The combined organic layers were washed with brine (5 mL), dried over anhydrous sodium sulfate, filtered and the filtrate was concentrated under reduced pressure. The residue was purified by column chromatography to afford the title compound. LCMS R_{T}=1.278 min, m/z=475.0 [M+H]⁺

### Step 3: (2R,4S)-tert-butyl 4-(8-(2-(((tert-butyldimethylsilyl)oxy)methyl)thieno[3,2-b]pyridin-7-yl)-6-chloro-3,4-dihydroquinolin-1(2H)-yl)-2-(methoxymethyl)-2-methylpyrrolidine-1-carboxylate

To a solution of (2R,4S)-tert-butyl 4-(8-bromo-6-chloro-3,4-dihydroquinolin-1(2H)-yl)-2-(methoxymethyl)-2-methylpyrrolidine-1-carboxylate (50 mg, 0.1 mmol),[2-[[tert-butyl(dimethyl)silyl]oxymethyl]thieno[3,2-b]pyridin-7-yl]boronic acid (41 mg, 0.13 mmol) and potassium carbonate (103 mg, 0.32 mmol) in dioxane (2 mL) and water (0.3 mL) was added dichloro[1,1′-bis(diphenylphosphino)ferrocene]palladium(II) (15 mg, 0.02 mmol). After stirred at 100° C. for 1 h under nitrogen atmosphere, the reaction mixture was filtered and the filtrate was concentrated under reduced pressure. The residue was purified by preparative TLC to afford the title compound. LCMS R_{T}=2.012 min, m/z=672.4 [M+H]⁺

### Step 4: (2R,4S)-tert-butyl 4-(6-chloro-8-(2-(hydroxymethyl)thieno[3,2-b]pyridin-7-yl)-3,4-dihydroquinolin-1(2H)-yl)-2-(methoxymethyl)-2-methylpyrrolidine-1-carboxylate

To a solution of (2R,4S)-tert-butyl 4-(8-(2-(((tert-butyldimethylsilyl)oxy)methyl)thieno[3,2-b]pyridin-7-yl)-6-chloro-3,4-dihydroquinolin-1(2H)-yl)-2-(methoxymethyl)-2-methylpyrrolidine-1-carboxylate (60 mg, 0.09 mmol) in THF (1 mL) was added tetrabutylammonium fluoride (1 M, 0.09 mL). After stirred at 20° C. for 1 h under nitrogen atmosphere, the reaction mixture was concentrated under reduced pressure. The residue was purified by preparative TLC to afford the title compound. LCMS R_{T}-2.187 min, m/z=557.2 [M+H]⁺

### Step 5

Example 200 was prepared from (2R,4S)-tert-butyl 4-(6-chloro-8-(2-(hydroxymethyl)thieno[3,2-b]pyridin-7-yl)-3,4-dihydroquinolin-1(2H)-yl)-2-(methoxymethyl)-2-methylpyrrolidine-1-carboxylate and succinimide, following the procedure described in the synthesis of Example 108. The product was purified by preparative TLC. ¹H NMR (400 MHz, CD₃OD) δ=8.63 (d, J=4.8 Hz, 1H), 7.43-7.31 (m, 2H), 7.12-7.00 (m, 2H), 4.85 (s, 2H), 3.65 (s, 1H), 3.27-3.21 (m, 2H), 3.13-3.04 (m, 3H), 2.90-2.54 (m, 10H), 2.00-1.28 (m, 4H), 1.12 (m, 3H). LCMS R_{T}=0.455 min, m/z=539.2 [M+H]⁺

### Example 201: Methyl (2R,4R)-4-(6-chloro-8-(2-((2,5-dioxopyrrolidin-1-yl)methyl)thieno[3,2-b]pyridin-7-yl)-3,4-dihydroquinolin-1(2H)-yl)-2-methylpyrrolidine-2-carboxylate

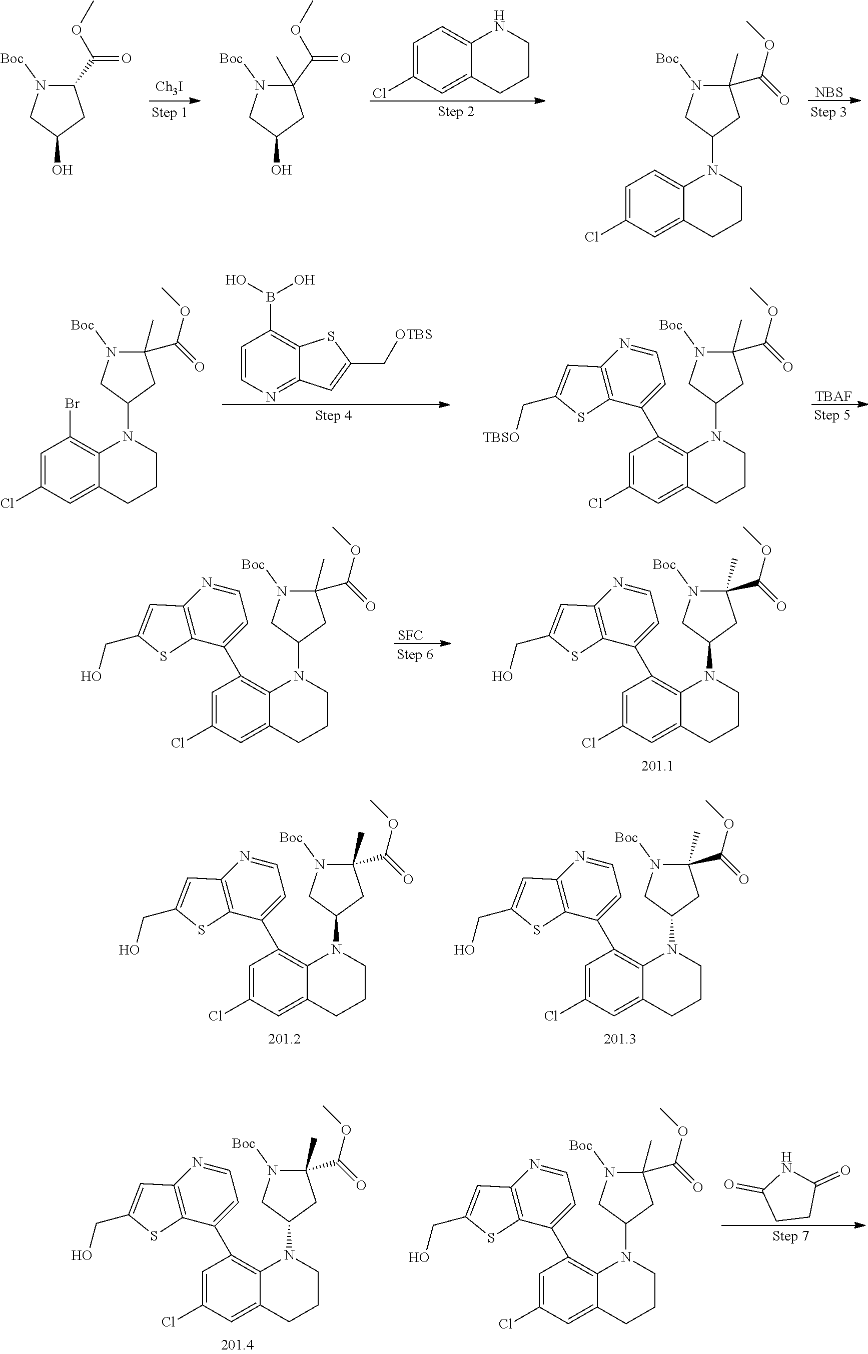

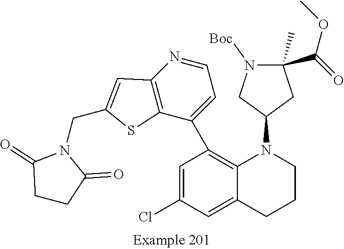 [SMILES: COC(=O)[C@@]1(C)C[C@@H](N2CCCc3cc(Cl)cc(-c4ccnc5cc(CN6C(=O)CCC6=O)sc45)c32)CN1C(=O)OC(C)(C)C]

### Step 1: (4R)-1-tert-butyl 2-methyl 4-hydroxy-2-methylpyrrolidine-1,2-dicarboxylate

To a solution of (2S,4R)-1-tert-butyl 2-methyl 4-hydroxypyrrolidine-1,2-dicarboxylate (19.0 g, 79.10 mmol) in THF (150 mL) was added diisopropylamino lithium (2 M, 148 mL), then methyl iodide (44.0 g, 316.38 mmol) was dropwise added at −40° C. After stirred at 25° C. for 12 h, the reaction mixture was quenched by addition of ammonium chloride (100 mL), and then extracted with ethyl acetate (50 mL×3). The combined organic layers were dried over sodium sulphate, filtered and the filtrate was concentrated under reduced pressure to give a residue. The residue was purified by column chromatography to afford the title compound. LCMS R_{T}=1.356 min, m/z=260.4 [M+H]⁺.

### Step 2: 1-tert-butyl 2-methyl 4-(6-chloro-3,4-dihydroquinolin-1(2H)-yl)-2-methylpyrrolidine-1,2-dicarboxylate

To a solution of (4R)-1-tert-butyl 2-methyl 4-hydroxy-2-methylpyrrolidine-1,2-dicarboxylate (7.0 g, 27.00 mmol) in dichloromethane (50 mL) was added trifluoromethylanhydride (9.0 g, 32.40 mmol) and diisopropyl ethyl amine (5.0 g, 40.49 mmol) and 6-chloro-1,2,3,4-tetrahydroquinoline (5.0 g, 27.00 mmol) at −65° C. After stirred at 25° C. for 12 h, the reaction mixture was quenched by addition sodium bicarbonate (100 mL), and then extracted with dichloromethane (50 mL×3). The combined organic layers were dried over sodium sulphate, filtered and the filtrate was concentrated under reduced pressure to give a residue. The residue was purified by column chromatography to afford the title compound. LCMS R_{T}=0.658 min, m/z=409.3 [M+H]⁺.

### Step 3: 1-tert-butyl 2-methyl 4-(8-bromo-6-chloro-3,4-dihydroquinolin-1(2H)-yl)-2-methylpyrrolidine-1,2-dicarboxylate

To a solution of 1-tert-butyl 2-methyl 4-(6-chloro-3,4-dihydroquinolin-1(2H)-yl)-2-methylpyrrolidine-1,2-dicarboxylate (1.0 g, 2.45 mmol) in dichloromethane (10 mL) was added N-bromosuccinimide (653 mg, 3.67 mmol) at 25° C. After stirred at 25° C. for 3 h, the reaction mixture was quenched by addition of ammonium chloride (30 mL), and then extracted with dichloromethane (10 mL×3). The combined organic layers were dried over sodium sulphate, filtered and the filtrate was concentrated under reduced pressure to give a residue. The residue was purified by column chromatography to afford the title compound. LCMS R_{T}=0.670 min, m/z=489.0 [M+H]⁺.

### Step 4: 1-tert-butyl 2-methyl 4-(8-(2-(((tert-butyldimethylsilyl)oxy)methyl)thieno[3,2-b]pyridin-7-yl)-6-chloro-3,4-dihydroquinolin-1(2H)-yl)-2-methylpyrrolidine-1,2-dicarboxylate

To a solution of 1-tert-butyl 2-methyl 4-(8-bromo-6-chloro-3,4-dihydroquinolin-1(2H)-yl)-2-methylpyrrolidine-1,2-dicarboxylate (900 mg, 1.84 mmol) in dioxane (10 mL) and water (5 mL) was added [2-[[tert-butyl(dimethyl)silyl]oxymethyl]thieno[3,2-b]pyridin-7-yl]boronic acid (895 mg, 2.77 mmol) and dichloro[1,1′-bis(diphenylphosphino)ferrocene]palladium(II) (270 mg, 0.37 mmol) and cesium carbonate (2.0 g, 5.53 mmol) under nitrogen atmosphere. After stirred at 120° C. for 1 h, the reaction mixture was diluted with water (30 mL), extracted with ethyl acetate (30 mL×2). The combined organic layers were washed with brine (30 mL), dried over anhydrous sodium sulfate and concentrated under reduced pressure. The residue was purified by column chromatography to afford the title compound. LCMS R_{T}=3.253 min, m/z=686.2 [M+H]⁺.

### Step 5: 1-tert-butyl 2-methyl 4-(6-chloro-8-(2-(hydroxymethyl)thieno[3,2-b]pyridin-7-yl)-3,4-dihydroquinolin-1(2H)-yl)-2-methylpyrrolidine-1,2-dicarboxylate

To a solution of 1-tert-butyl 2-methyl 4-(8-(2-(((tert-butyldimethylsilyl)oxy)methyl)thieno[3,2-b]pyridin-7-yl)-6-chloro-3,4-dihydroquinolin-1(2H)-yl)-2-methylpyrrolidine-1,2-dicarboxylate (1.0 g, 1.46 mmol) in THF (10 mL) was added tetrabutylammonium fluoride (1 M, 3 ml) at 25° C. After stirred at 25° C. for 30 min, the combined organic layers were washed with brine (30 mL), dried over anhydrous sodium sulfate and concentrated under reduced pressure. The residue was purified by column chromatography to afford the title compound. LCMS R_{T}=0.547 min, m/z=572.2 [M+H]⁺.

### Step 6

The racemate was separated by SFC to give first eluting fraction (201.1, LCMS R_{T}=0.925 min, m/z=572.1 [M+H]+), second eluting fraction (201.2, LCMS R_{T}=0.925 min, m/z=572.1 [M+H]+), third eluting fraction (201.3, LCMS R_{T}=0.925 min, m/z=572.1 [M+H]+), and fourth eluting fraction (201.4, LCMS R_{T}=0.925 min, m/z=572.1 [M+H]+).

Example 201 was prepared from 201.1 and succinimide, following the procedure described in the synthesis of Example 108. The product was purified by HPLC. ¹H NMR (400 MHz, CD₃OD) δ=8.29-8.33 (m, 1H), 7.12 (s, 1H), 7.07 (d, J=4.8 Hz, 1H), 6.70-6.78 (m, 2H), 4.54-4.59 (m, 2H), 3.13-3.23 (m, 1H), 2.93 (dt, J=3.24, 1.63 Hz, 2H), 2.76 (s, 1H), 2.56 (m, 1H), 2.46 (t, J=6.0 Hz, 4H), 2.37 (s, 4H), 0.77-1.59 (m, 4H), 0.49 (s, 3H). LCMS R_{T}=1.183, m/z=553.4 [M+H]⁺.

### Example 202: Methyl (2S,4R)-4-(6-chloro-8-(2-((2,5-dioxopyrrolidin-1-yl)methyl)thieno[3,2-b]pyridin-7-yl)-3,4-dihydroquinolin-1(2H)-yl)-2-methylpyrrolidine-2-carboxylate

Example 202 was prepared from 201.2 and succinimide, following the procedure described in the synthesis of Example 108. The product was purified by HPLC. ¹H NMR (400 MHz, CD₃OD) δ=8.70-8.64 (m, 1H), 7.51-7.47 (m, 1H), 7.39 C 7.34 (m, 1H), 7.15-7.06 (m, 2H), 4.98-4.92 (m, 2H), 3.51 (s, 3H), 3.47-3.36 (m, 1H), 3.28-3.14 (m, 2H), 2.89-2.80 (m, 1H), 2.94-2.26 (m, 7H), 1.98-1.40 (m, 4H), 1.31-1.22 (m, 3H). LCMS R_{T}=0.462 min, m/z=553.2[M+H]⁺.

### Example 203: Methyl (2R,4S)-4-(6-chloro-8-(2-((2,5-dioxopyrrolidin-1-yl)methyl)thieno[3,2-b]pyridin-7-yl)-3,4-dihydroquinolin-1(2H)-yl)-2-methylpyrrolidine-2-carboxylate

Example 203 was prepared from 201.3 and succinimide, following the procedure described in the synthesis of Example 108. The product was purified by HPLC. ¹H NMR (400 MHz, CD₃OD) δ=8.61-8.39 (m, 1H), 7.42-7.19 (m, 2H), 7.00-6.75 (m, 2H), 4.72 (s, 2H), 3.47 (s, 3H), 3.42-3.26 (m, 1H), 2.91 (br. s, 2H), 2.69-2.23 (m, 8H), 1.78-1.51 (m, 3H), 0.96-0.29 (m, 4H) LCMS R_{T}=1.237 min, m/z=553.4 [M+H]+

### Example 204: Methyl (2R,4R)-4-[6-chloro-8-[2-[(2,5-dioxopyrrolidin-1-yl)methyl]thieno[3,2-b]pyridin-7-yl]-3,4-dihydro-2H-quinolin-1-yl]-2-methyl-pyrrolidine-2-carboxylate

Example 204 was prepared from 201.4 and succinimide, following the procedure described in the synthesis of Example 108. The product was purified by HPLC. ¹H NMR (400 MHz, CD₃OD) δ=8.67 (d, J=5.2 Hz, 1H), 7.50 (s, 1H), 7.37 (d, J=4.8 Hz, 1H), 7.11 (dd, J=2.4, 14.9 Hz, 2H), 4.99-4.92 (m, 2H), 3.50 (s, 3H), 3.45-3.36 (m, 1H), 3.28-3.13 (m, 2H), 2.95-2.31 (m, 8H), 2.01-1.79 (m, 2H), 1.70-0.93 (m, 5H). LCMS R_{T}=0.454 min, m/z=553.3 [M+H]⁺.

### Example 205: (2S,4S)-4-(6-chloro-8-(2-((2,5-dioxopyrrolidin-1-yl)methyl)thieno[3,2-b]pyridin-7-yl)-3,4-dihydroquinolin-1(2H)-yl)-2-methylpyrrolidine-2-carboxylic acid

A solution of methyl (2S,4S)-4-[6-chloro-8-[2-[(2,5-dioxopyrrolidin-1-yl)methyl]thieno[3,2-b]pyridin-7-yl]-3,4-dihydro-2H-quinolin-1-yl]-2-methyl-pyrrolidine-2-carboxylate (7 mg, 0.01 mmol) in hydrochloric acid (1 mL, 6 M in water) was stirred at 80° C. for 1 h. The mixture was concentrated under reduced pressure. The residue was purified by preparative HPLC to afford the title compound. ¹H NMR (400 MHz, CD₃OD) δ=8.88-8.58 (m, 1H), 7.64-7.40 (m, 2H), 7.29-7.02 (m, 2H), 4.97 (s, 2H), 3.72 (d, J=4.4 Hz, 1H), 3.26-3.12 (m, 2H), 2.99-2.59 (m, 8H), 2.20-1.57 (m, 4H), 1.16-0.89 (m, 3H). LCMS R_{T}=1.368 min, m/z=539.4 [M+H]⁺.

### Example 206: 8-(2-((2,4-dioxo-3-azabicyclo[3.1.0]hexan-3-yl)methyl)thieno[3,2-b]pyridin-7-yl)-1-((3S,5R)-5-(hydroxymethyl)-5-methylpyrrolidin-3-yl)-1,2,3,4-tetrahydroquinoline-6-carbonitrile, formic acid salt

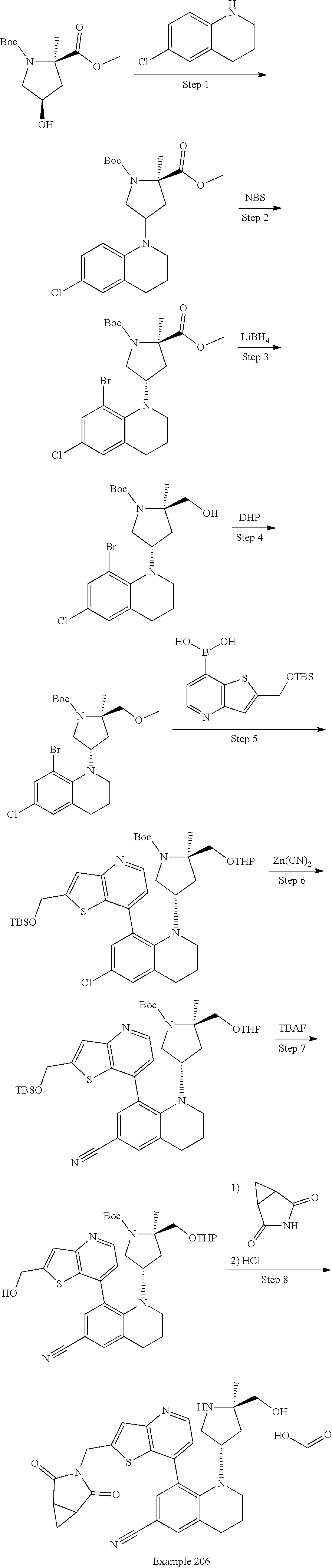

### Step 1: (2R)-1-tert-butyl 2-methyl 4-(6-chloro-3,4-dihydroquinolin-1(2H)-yl)-2-methylpyrrolidine-1,2-dicarboxylate

To a solution of (2R,4R)-1-tert-butyl 2-methyl 4-hydroxy-2-methylpyrrolidine-1,2-dicarboxylate (20 mL) were added N-ethyl-N-isopropylpropan-2-amine (2.3 g, 17.35 mmol), trifluoromethylsulfonyl trifluoromethanesulfonate (3.9 g, 13.88 mmol) and 6-chloro-1,2,3,4-tetrahydroquinoline (3.3 g, 19.67 mmol) at −65° C. After stirred at 25° C. for 3 h, the reaction mixture was concentrated under reduced pressure. Then the concentrate was quenched by addition of saturated sodium bicarbonate (40 mL) and extracted with ethyl acetate (30 mL×3). The combined organic layers were washed with brine (60 mL), dried over anhydrous sodium sulfate and concentrated under reduced pressure. The residue was purified by column chromatography to give the title compound. LCMS R_{T}=0.647 min, m/z=409.1 [M+H]⁺.

### Step 2: (2R,4S)-1-tert-butyl 2-methyl 4-(8-bromo-6-chloro-3,4-dihydroquinolin-1(2H)-yl)-2-methylpyrrolidine-1,2-dicarboxylate

To a solution of (2R)-1-tert-butyl 2-methyl 4-(6-chloro-3,4-dihydroquinolin-1(2H)-yl)-2-methylpyrrolidine-1,2-dicarboxylate (3.0 g, 7.34 mmol) in dichloromethane (20 mL) was added N-bromosuccinimide (1.3 g, 6.97 mmol). After stirred for 1 h at 25° C., the residue was diluted with saturated aqueous ammonium chloride (10 mL), extracted with dichloromethane (15 mL×2). The combined organic layers were washed with brine (20 mL), dried over anhydrous sodium sulfate and concentrated under reduced pressure to afford the title crude compound. It was used in next step without further purification. LCMS R_{T}=0.672 min, m/z=489.0 [M+H]⁺.

### Step 3: (2R,4S)-tert-butyl 4-(8-bromo-6-chloro-3,4-dihydroquinolin-1(2H)-yl)-2-(hydroxymethyl)-2-methylpyrrolidine-1-carboxylate

To a solution of (2R,4S)-1-tert-butyl 2-methyl 4-(8-bromo-6-chloro-3,4-dihydroquinolin-1(2H)-yl)-2-methylpyrrolidine-1,2-dicarboxylate (1.0 g, 2.05 mmol) in THF (10 mL) was added lithium borohydride (180 mg, 8.20 mmol) at 0° C. After stirred at 25° C. for 12 h, the reaction mixture was concentrated under reduced pressure. The residue was quenched by addition of saturated aqueous ammonium chloride (30 mL) and extracted with ethyl acetate (50 mL×3). The combined organic layers were washed with saturated ammonium chloride (50 mL×2), dried over anhydrous sodium sulfate and concentrated under reduced pressure. The residue was purified by column chromatography to give the title compound. LCMS R_{T}=0.963 min, m/z=461.1 [M+H]⁺.

### Step 4: (2R,4S)-tert-butyl 4-(8-bromo-6-chloro-3,4-dihydroquinolin-1(2H)-yl)-2-methyl-2-(((tetrahydro-2H-pyran-2-yl)oxy)methyl)pyrrolidine-1-carboxylate

To a solution of (2R,4S)-tert-butyl 4-(8-bromo-6-chloro-3,4-dihydroquinolin-1(2H)-yl)-2-(hydroxymethyl)-2-methylpyrrolidine-1-carboxylate (750 mg, 1.63 mmol) in acetone (8 mL) was added (3,4-dihydro-2H-pyran-2-yl)methanol (610 mg, 4.89 mmol) and pyridinium p-toluenesulfonate (125 mg, 0.49 mmol). After stirred at 50° C. for 2 h, the reaction mixture was concentrated under reduced pressure. The residue was purified by column chromatography to give the title compound. LCMS R_{T}=2.220 min, m/z=545.2 [M+H]⁺.

### Step 5: (2R,4S)-tert-butyl 4-(8-(2-(((tert-butyldimethylsilyl)oxy)methyl)thieno[3,2-b]pyridin-7-yl)-6-chloro-3,4-dihydroquinolin-1(2H)-yl)-2-methyl-2-(((tetrahydro-2H-pyran-2-yl)oxy)methyl)pyrrolidine-1-carboxylate

To a solution of (2R,4S)-tert-butyl 4-(8-bromo-6-chloro-3,4-dihydroquinolin-1(2H)-yl)-2-methyl-2-(((tetrahydro-2H-pyran-2-yl)oxy)methyl)pyrrolidine-1-carboxylate (900 mg, 1.65 mmol) in dioxane (10 mL) and water (2 mL) were added cesium carbonate (1.1 g, 3.31 mmol), [2-[[tert-butyl(dimethyl)silyl]oxymethyl]thieno[3,2-b]pyridin-7-yl]boronic acid (535 mg, 1.65 mmol) and dichloro[1,1′-bis(diphenylphosphino)ferrocene]palladium(II) (242 mg, 0.33 mmol). After stirred at 120° C. for 2 h under nitrogen atmosphere, the residue was diluted with saturated aqueous ammonium chloride (15 mL), extracted with dichloromethane (15 mL×3). The combined organic layers were washed with brine (15 mL), dried over anhydrous sodium sulfate and concentrated under reduced pressure. The residue was purified by column chromatography to give the title compound. LCMS R_{T}=1.635 min, m/z=742.3 [M+H]⁺.

### Step 6: (2R,4S)-tert-butyl 4-(8-(2-(((tert-butyldimethylsilyl)oxy)methyl)thieno[3,2-b]pyridin-7-yl)-6-cyano-3,4-dihydroquinolin-1(2H)-yl)-2-methyl-2-(((tetrahydro-2H-pyran-2-yl)oxy)methyl)pyrrolidine-1-carboxylate

To a solution of (2R,4S)-tert-butyl 4-(8-(2-(((tert-butyldimethylsilyl)oxy)methyl)thieno[3,2-b]pyridin-7-yl)-6-chloro-3,4-dihydroquinolin-1(2H)-yl)-2-methyl-2-(((tetrahydro-2H-pyran-2-yl)oxy)methyl)pyrrolidine-1-carboxylate (330 mg, 0.44 mmol) in 1-methylpyrrolidin-2-one (2 mL) were added zinc cyanide (522 mg, 4.44 mmol) and bis(tri-tert-butylphosphine) palladium(0) (45 mg, 0.09 mmol). After stirred at 95° C. for 12 h under nitrogen atmosphere, the reaction mixture was diluted with water (3 mL) and extracted with ethyl acetate (15 mL×3). The combined organic layers were washed with brine (15 mL), dried over anhydrous sodium sulfate, filtered and the filtrate was concentrated under reduced pressure. The residue was purified by column chromatography to give the title compound. LCMS R_{T}=0.987 min, m/z=733.5 [M+H]⁺.

### Step 7: (2R,4S)-tert-butyl 4-(6-cyano-8-(2-(hydroxymethyl)thieno[3,2-b]pyridin-7-yl)-3,4-dihydroquinolin-1(2H)-yl)-2-methyl-2-(((tetrahydro-2H-pyran-2-yl)oxy)methyl)pyrrolidine-1-carboxylate

To a solution of (2R,4S)-tert-butyl 4-(8-(2-(((tert-butyldimethylsilyl)oxy)methyl)thieno[3,2-b]pyridin-7-yl)-6-cyano-3,4-dihydroquinolin-1(2H)-yl)-2-methyl-2-(((tetrahydro-2H-pyran-2-yl)oxy)methyl)pyrrolidine-1-carboxylate (150 mg, 0.20 mmol) in THF (10 mL) were added tetrabutylammonium fluoride (0.3 mL, 1 M in THF). After stirred for 0.5 h at 25° C., the residue was concentrated under reduced pressure. The residue was purified by column chromatography to give the title compound. LCMS R_{T}=0.535 min, m/z=619.3 [M+H]⁺

### Step 8

Example 206 was prepared from (2R,4S)-tert-butyl 4-(6-cyano-8-(2-(hydroxymethyl)thieno[3,2-b]pyridin-7-yl)-3,4-dihydroquinolin-1(2H)-yl)-2-methyl-2-(((tetrahydro-2H-pyran-2-yl)oxy)methyl)pyrrolidine-1-carboxylate and 3-azabicyclo[3.1.0]hexane-2,4-dione (13 mg, 0.12 mmol), following the procedure described in the synthesis of Example 108. The product was purified by preparative HPLC. ¹H NMR (400 MHz, CD₃OD) δ=8.73 (d, J=4.8 Hz, 1H), 8.50 (s, 1H), 7.48 (t, J=5.2 Hz, 3H), 7.44 (s, 1H), 4.80 (s, 2H), 4.13-3.80 (m, 1H), 3.23-2.66 (m, 7H), 2.58 (dd, J=3.6, 8.4 Hz, 2H), 2.05-1.84 (m, 3H), 1.76-1.57 (m, 2H), 1.41-1.16 (m, 5H). LCMS R_{T}=1.290 min, m/z=528.2 [M+H]+

### Example 207: 2-((7-(6-chloro-1-((3S,5R)-5-(hydroxymethyl)-5-methylpyrrolidin-3-yl)-1,2,3,4-tetrahydroquinolin-8-yl)thieno[3,2-b]pyridin-2-yl)methyl)-5-(1-(trifluoromethyl)cyclopropyl)pyridazin-3(2H)-one, formic acid salt

Example 207 was prepared from (2R,4S)-tert-butyl 4-(6-chloro-8-(2-(hydroxymethyl)thieno[3,2-b]pyridin-7-yl)-3,4-dihydroquinolin-1(2H)-yl)-2-methyl-2-(((tetrahydro-2H-pyran-2-yl)oxy)methyl)pyrrolidine-1-carboxylate and 5-(1-(trifluoromethyl)cyclopropyl)pyridazin-3(2H)-one, following the procedure described in the synthesis of Example 108. The product was purified by HPLC. ¹H NMR (400 MHz, CD₃OD) δ=8.70 (d, J=4.8 Hz, 1H), 8.05 (d, J=1.6 Hz, 1H), 7.55 (s, 1H), 7.43 (d, J=4.8 Hz, 1H), 7.18-7.11 (m, 2H), 7.07 (d, J=2.0 Hz, 1H), 5.62 (s, 2H), 3.67 (s, 1H), 3.21-3.09 (m, 3H), 3.05-2.77 (m, 5H), 1.97-1.80 (m, 2H), 1.54-1.34 (m, 3H), 1.32-1.10 (m, 6H). LCMS R_{T}=0.850 min, m/z=630.1 [M+H]⁺.

### Example 208: 7-(6-chloro-1-((3S,5R)-5-(hydroxymethyl)-5-methylpyrrolidin-3-yl)-1,2,3,4-tetrahydroquinolin-8-yl)-2-((2,5-dioxopyrrolidin-1-yl)methyl)thieno[3,2-b]pyridine 4-oxide, formic acid salt

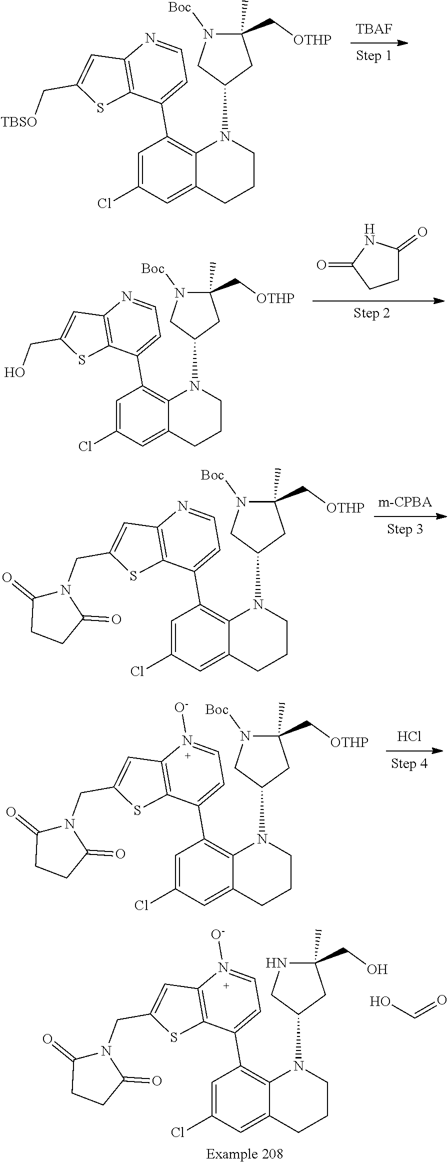

### Step 1: (2R,4S)-tert-butyl 4-(6-chloro-8-(2-(hydroxymethyl)thieno[3,2-b]pyridin-7-yl)-3,4-dihydroquinolin-1(2H)-yl)-2-methyl-2-(((tetrahydro-2H-pyran-2-yl)oxy)methyl)pyrrolidine-1-carboxylate

To a solution of (2R,4S)-tert-butyl 4-(8-(2-(((tert-butyldimethylsilyl)oxy)methyl)thieno[3,2-b]pyridin-7-yl)-6-chloro-3,4-dihydroquinolin-1(2H)-yl)-2-methyl-2-(((tetrahydro-2H-pyran-2-yl)oxy)methyl)pyrrolidine-1-carboxylate (150 mg, 0.20 mmol) in THF (10 mL) were added tetrabutylammonium fluoride (0.3 mL, 1 M in THF). After stirred for 0.5 h at 25° C., the residue was concentrated under reduced pressure. The residue was purified by column chromatography to give the title compound. LCMS R_{T}=0.849 min, m/z=628.2 [M+H]⁺

### Step 2: (2R,4S)-tert-butyl 4-(6-chloro-8-(2-((2,5-dioxopyrrolidin-1-yl)methyl)thieno[3,2-b]pyridin-7-yl)-3,4-dihydroquinolin-1(2H)-yl)-2-methyl-2-(((tetrahydro-2H-pyran-2-yl)oxy)methyl)pyrrolidine-1-carboxylate

To a solution of (2R,4S)-tert-butyl 4-(6-chloro-8-(2-(hydroxymethyl)thieno[3,2-b]pyridin-7-yl)-3,4-dihydroquinolin-1(2H)-yl)-2-methyl-2-(((tetrahydro-2H-pyran-2-yl)oxy)methyl)pyrrolidine-1-carboxylate (50 mg, 0.08 mmol) in toluene (2 mL) were added 3-azabicyclo[3.1.0]hexane-2,4-dione (13 mg, 0.12 mmol), (3E)-3-(dimethylcarbamoylimino)-1,1-dimethyl-urea (42 mg, 0.24 mmol) and tributylphosphane (98 mg, 0.48 mmol) at 25° C. After stirred at 100° C. for 1 h, the reaction mixture was concentrated under reduced pressure. The residue was purified by preparative HPLC to give the title compound. LCMS R_{T}=0.595 min, m/z=709.4 [M+H]⁺

### Step 3: tert-butyl (2R,4S)-4-[6-chloro-8-[2-[(2,5-dioxopyrrolidin-1-yl)methyl]-4-oxido-thieno[3,2-b]pyridin-4-ium-7-yl]-3,4-dihydro-2H-quinolin-1-yl]-2-methyl-2-(tetrahydropyran-2-yloxymethyl)pyrrolidine-1-carboxylate

To a solution of (2R,4S)-tert-butyl 4-(6-chloro-8-(2-((2,5-dioxopyrrolidin-1-yl)methyl)thieno[3,2-b]pyridin-7-yl)-3,4-dihydroquinolin-1(2H)-yl)-2-methyl-2-(((tetrahydro-2H-pyran-2-yl)oxy)methyl)pyrrolidine-1-carboxylate (50 mg, 0.14 mmol) in dichloromethane (2 mL) was added 3-Chloroperbenzoic acid (25 mg, 292.54 mmol). After stirred at 0° C. for 16 h, the reaction mixture was diluted with saturated sodium bicarbonate (10 mL), the filtrate extracted with ethyl acetate (10 mL×2). The combined organic layers were washed with saturated sodium thiosulfate (20 mL), dried over anhydrous sodium sulfate and concentrated under reduced pressure. The residue was purified by preparative HPLC to give the title compound. LCMS R_{T}=0.670 min, m/z=725.5 [M+H]⁺.

### Step 4

To a solution of tert-butyl (2R,4S)-4-[6-chloro-8-[2-[(2,5-dioxopyrrolidin-1-yl)methyl]-4-oxido-thieno[3,2-b]pyridin-4-ium-7-yl]-3,4-dihydro-2H-quinolin-1-yl]-2-methyl-2-(tetrahydropyran-2-yloxymethyl)pyrrolidine-1-carboxylate (30 mg, 0.04 mmol) was dissolved in hydrochloric acid (4 mL, 4 M in dioxane). After stirred at 25° C. for 0.5 h, the reaction mixture was concentrated under reduced pressure. The residue was purified by preparative HPLC to give Example 211. ¹H NMR (400 MHz, CD₃OD) δ=8.52-8.43 (m, 2H), 7.54-7.48 (m, 1H), 7.20 (s, 2H), 4.96 (s, 2H), 3.83 (br s, 1H), 3.29-2.97 (m, 6H), 2.94-2.71 (m, 8H), 2.01-1.84 (m, 2H), 1.29-1.23 (m, 3H). LCMS R_{T}=1.287 min, m/z=541.4 [M+H]⁺

### Example 209: 8-(2-((2,5-dioxopyrrolidin-1-yl)methyl)thieno[3,2-b]pyridin-7-yl)-1-((3S,5R)-5-(hydroxymethyl)-5-methylpyrrolidin-3-yl)-1,2,3,4-tetrahydroquinoline-6-carbonitrile, formic acid salt Example 209 was prepared from (2R,4S)-tert-butyl 4-(6-cyano-8-(2-(hydroxymethyl)thieno[3,2-b]pyridin-7-yl)-3,4-dihydroquinolin-1(2H)-yl)-2-methyl-2-(((tetrahydro-2H-pyran-2-yl)oxy)methyl)pyrrolidine-1-carboxylate and succinimide, following the procedure described in the synthesis of Example 108. The product was purified by preparative HPLC. ¹H NMR (400 MHz, CD₃OD) δ=8.72 (d, J=4.8 Hz, 1H), 7.50-7.44 (m, 4H), 4.94 (s, 2H), 4.15-3.84 (m, 1H), 3.25-2.59 (m, 11H), 2.02-1.61 (m, 4H), 1.49-1.19 (m, 4H). LCMS R_{T}=1.113, m/z=516.4 [M+H]⁺

### Example 210: 1-[[7-[6-chloro-1-[(3S,5R)-5-(hydroxymethyl)-5-methyl-pyrrolidin-3-yl]-3,4-dihydro-2H-quinolin-8-yl]-3-fluoro-thieno[3,2-b]pyridin-2-yl]methyl]pyrrolidine-2,5-dione, formic acid salt

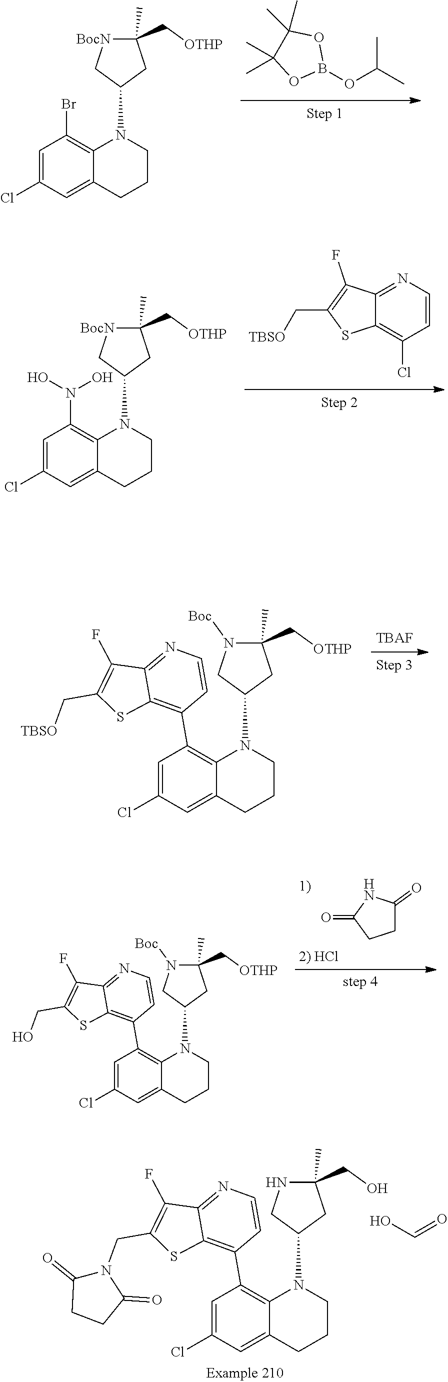

### Step 1: [1-[(3S,5R)-1-tert-butoxycarbonyl-5-methyl-5-(tetrahydropyran-2-yloxymethyl)pyrrolidin-3-yl]-6-chloro-3,4-dihydro-2H-quinolin-8-yl]boronic acid

To a solution of (2R,4S)-tert-butyl 4-(8-bromo-6-chloro-3,4-dihydroquinolin-1(2H)-yl)-2-methyl-2-(((tetrahydro-2H-pyran-2-yl)oxy)methyl)pyrrolidine-1-carboxylate (1.3 M in THF, 3.8 mL) in THF (3 mL) was added tert-butyl (2R,4S)-4-(8-bromo-6-chloro-3,4-dihydro-2H-quinolin-1-yl)-2-methyl-2-(tetrahydropyran-2-yloxymethyl)pyrrolidine-1-carboxylate (500 mg, 0.91 mmol) and 2-isopropoxy-4,4,5,5-tetramethyl-1,3,2-dioxaborolane (342 mg, 1.84 mmol) at 0° C. After stirred at 0° C. for 1 h, the mixture was quenched by addition of saturated aqueous ammonium chloride (10 mL) and extracted with ethyl acetate (10 mL). The organic layer washed with brine (10 mL), dried over sodium sulphate and concentrated. The residue was purified by column chromatography to afford the title compound. LCMS R_{T}=2.215 min, m/z=509.2 [M+H]⁺.

### Step 2: tert-butyl (2R,4S)-4-[8-[2-[[tert-butyl(dimethyl)silyl]oxymethyl]-3-fluoro-thieno[3,2-b]pyridin-7-yl]-6-chloro-3,4-dihydro-2H-quinolin-1-yl]-2-methyl-2-(tetrahydropyran-2-yloxymethyl)pyrrolidine-1-carboxylate

To a solution of [1-[(3S,5R)-1-tert-butoxycarbonyl-5-methyl-5-(tetrahydropyran-2-yloxymethyl)pyrrolidin-3-yl]-6-chloro-3,4-dihydro-2H-quinolin-8-yl]boronic acid (500 mg, 0.9 mmol) in dioxane (2 mL) and water (0.5 mL) was added tert-butyl-[(7-chloro-3-fluoro-thieno[3,2-b]pyridin-2-yl)methoxy]-dimethyl-silane (326 mg, 0.9 mmol) and cyclopentyl(diphenyl)phosphane dichloropalladium iron (145 mg, 0.1 mmol) and cesium carbonate (960 mg, 2.95 mmol). After stirred at 120° C. for 2 h, the reaction mixture was quenched by addition saturated aqueous ammonium chloride (10 mL), and then diluted with water (15 mL) and extracted with ethyl acetate (5 mL×3). The combined organic layers were washed with brine (15 mL), dried over anhydrous sodium sulfate, filtered and the filtrate was concentrated under reduced pressure to give a residue. The residue was purified by column chromatography to afford the title compound. LCMS R_{T}=2.570 min, m/z=760.6 [M+H]⁺.

### Step 3: tert-butyl (2R,4S)-4-[6-chloro-8-[3-fluoro-2-(hydroxymethyl)thieno[3,2-b]pyridin-7-yl]-3,4-dihydro-2H-quinolin-1-yl]-2-methyl-2-(tetrahydropyran-2-yloxymethyl)pyrrolidine-1-carboxylate

To a solution of tert-butyl (2R,4S)-4-[8-[2-[[tert-butyl(dimethyl)silyl]oxymethyl]-3-fluoro-thieno[3,2-b]pyridin-7-yl]-6-chloro-3,4-dihydro-2H-quinolin-1-yl]-2-methyl-2-(tetrahydropyran-2-yloxymethyl)pyrrolidine-1-carboxylate (200 mg, 0.2 mmol) in THF (10 mL) was added tetrabutylammonium fluoride (1 M in THF, 3 mL). After stirred at 25° C. for 0.5 h, the reaction mixture was concentrated under reduced pressure. The residue was purified by column chromatography to afford the title compound. LCMS R_{T}=1.035 min, m/z=646.3 [M+H]⁺.

### Step 4

Example 210 was prepared from tert-butyl (2R,4S)-4-[6-chloro-8-[3-fluoro-2-(hydroxymethyl)thieno[3,2-b]pyridin-7-yl]-3,4-dihydro-2H-quinolin-1-yl]-2-methyl-2-(tetrahydropyran-2-yloxymethyl)pyrrolidine-1-carboxylate and succinimide, following the procedure described in the synthesis of Example 108. The product was purified by preparative HPLC. ¹H NMR (400 MHz, CD₃OD) δ=8.76 (d, J=4.8 Hz, 1H), 7.52 (d, J=4.8 Hz, 1H), 7.21-7.09 (m, 2H), 4.96 (s, 2H), 3.77 (s, 1H), 3.29-3.21 (m, 2H), 3.10 (s, 1H), 2.88 (t, J=6.4 Hz, 4H), 2.74 (s, 5H), 1.99-1.41 (m, 4H), 1.24 (s, 3H). LCMS R_{T}=1.453 min, m/z=543.4 [M+H]⁺.

### Example 211: 1-((7-(6-chloro-1-(2-fluoro-5-azaspiro[3.4]octan-7-yl)-1,2,3,4-tetrahydroquinolin-8-yl)thieno[3,2-b]pyridin-2-yl)methyl)pyrrolidine-2,5-dione, formic acid salt

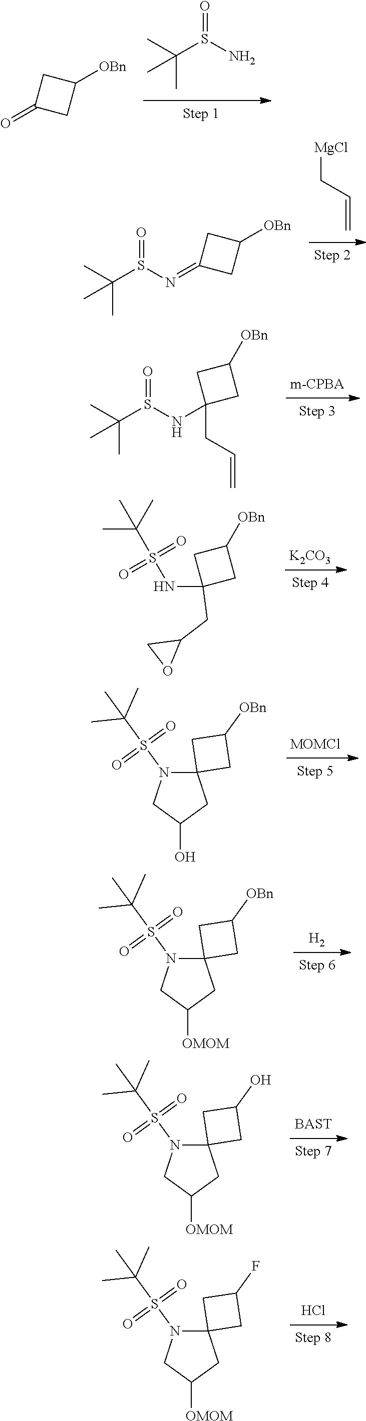

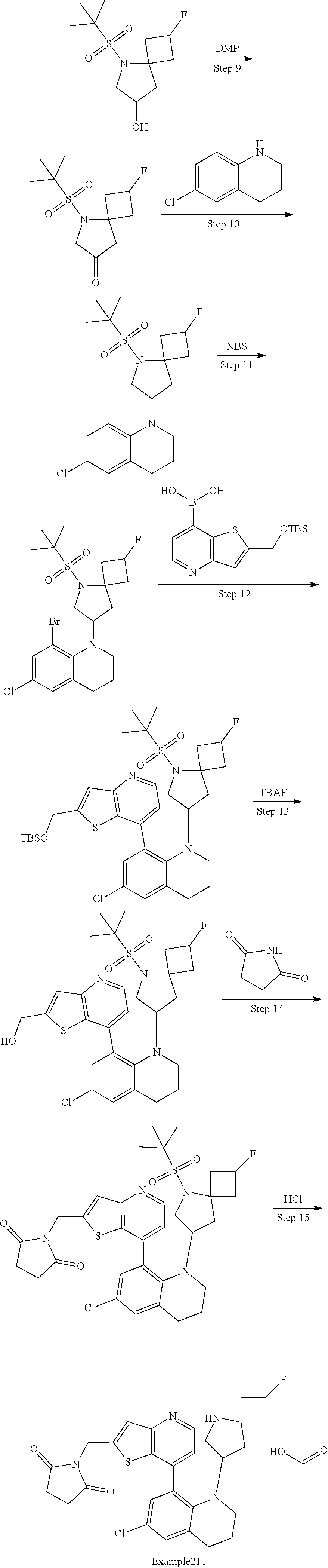

### Step 1: N-(3-(benzyloxy)cyclobutylidene)-2-methylpropane-2-sulfinamide

To a solution of 3-(benzyloxy)cyclobutanone (16 g, 90.80 mmol) in THF (200 mL) was added 2-methylpropane-2-sulfinamide (12.11 g, 99.88 mmol) and titanium(iv)isopropoxide (30.71 g, 108.05 mmol) at 25° C. After stirred at 50° C. for 12 h, the mixture was quenched by addition of water (50 mL) and filtered. The filtrate was diluted with water (200 mL) and extracted with ethyl acetate (100 mL×3). The combined organic layers were washed with brine (100 mL), dried over anhydrous sodium sulfate and concentrated under reduced pressure. The residue was purified by column chromatography to afford the title compound. LCMS R_{T}=0.523 min, m/z=280.1 [M+H]⁺.

### Step 2: N-(1-allyl-3-(benzyloxy)cyclobutyl)-2-methylpropane-2-sulfinamide

To a solution of allyl(chloro)magnesium (62.99 mL, 2 M in THF) was added N-(3-(benzyloxy)cyclobutylidene)-2-methylpropane-2-sulfinamide (22 g, 62.99 mmol) in THF (50 mL) at −40° C. After stirred at 25° C. for 6 h, the mixture was quenched by addition of saturated aqueous ammonium chloride (50 mL) and water (100 mL). The mixture was extracted with ethyl acetate (100 mL×2). The combined organic layers were washed with brine (100 mL), dried over anhydrous sodium sulfate and concentrated under reduced pressure. The residue was purified by column chromatography to afford the title compound. LCMS R_{T}=1.980 min, m/z=322.0 [M+H]⁺.

### Step 3: N-(3-(benzyloxy)-1-(oxiran-2-ylmethyl)cyclobutyl)-2-methylpropane-2-sulfonamide

To a solution of N-(1-allyl-3-(benzyloxy)cyclobutyl)-2-methylpropane-2-sulfinamide (11 g, 27.37 mmol) in dichloromethane (150 mL) was added 3-chlorobenzenecarboperoxoic acid (15.01 g, 73.91 mmol) at 0° C. After stirred at 25° C. for 12 h, the mixture was quenched by addition of saturated aqueous sodium hydrogencarbonate (150 mL) and extracted with dichloromethane (100 mL). The organic layer was washed with saturated aqueous sodium sulfite (40 mL) and brine (80 mL), dried over anhydrous sodium sulfate and concentrated under reduced pressure. The residue was purified by column chromatography to afford the title compound. LCMS R_{T}=2.289 min, m/z=354.2 [M+H]⁺.

### Step 4: 2-(benzyloxy)-5-(tert-butylsulfonyl)-5-azaspiro[3.4]octan-7-ol

To a solution of N-(3-(benzyloxy)-1-(oxiran-2-ylmethyl)cyclobutyl)-2-methylpropane-2-sulfonamide (10.4 g, 29.42 mmol) in DMF (100 mL) was added potassium iodide (4.88 g, 29.42 mmol) and potassium carbonate (12.2 g, 88.27 mmol). After stirred at 100° C. for 2.5 h, the reaction mixture was filtered and the filtrate was concentrated under reduced pressure. The residue was purified by column chromatography to afford the title compound. LCMS R_{T}=0.795 min, m/z=354.2 [M+H]⁺.

### Step 5: 2-(benzyloxy)-5-(tert-butylsulfonyl)-7-(methoxymethoxy)-5-azaspiro[3.4]octane

To a solution of 2-(benzyloxy)-5-(tert-butylsulfonyl)-5-azaspiro[3.4]octan-7-ol (6.4 g, 18.11 mmol) in THF (80 mL) was added sodium hydrogen (1.09 g, 27.17 mmol). After stirred at 25° C. for 0.5 h, chloro(methoxy)methane (2.9 g, 36.02 mmol) was added. After stirred at 45° C. for 2 h and 25° C. for 14 h, the mixture was quenched by addition of saturated aqueous sodium hydrogencarbonate (80 mL) and concentrated to remove the solvent. The mixture was extracted with ethyl acetate (60 mL×2). The combined organic layers were washed with brine (80 mL), dried over anhydrous sodium sulfate and concentrated under reduced pressure. The residue was purified by column chromatography to afford the title compound.

### Step 6: 5-(tert-butylsulfonyl)-7-(methoxymethoxy)-5-azaspiro[3.4]octan-2-ol

To a solution of 2-(benzyloxy)-5-(tert-butylsulfonyl)-7-(methoxymethoxy)-5-azaspiro[3.4]octane (5.2 g, 13.08 mmol) in methanol (90 mL) was added palladium 10% on activated carbon (2.77 g, 2.62 mmol) at 25° C. After stirred at 25° C. for 15 h under hydrogen atmosphere, the mixture was filtered and the filtrate was concentrated under reduced pressure. The residue was purified by column chromatography to afford the title compound. ¹H NMR (400 MHz, CD₃OD) δ=4.69-4.59 (m, 2H), 4.39-3.92 (m, 2H), 3.68 (dt, J=6.0, 10.8 Hz, 1H), 3.50 (td, J=3.6, 10.4 Hz, 1H), 3.38-3.33 (m, 3H), 3.19-2.80 (m, 2H), 2.60-2.09 (m, 4H), 1.41-1.35 (m, 9H).

### Step 7: 5-(tert-butylsulfonyl)-2-fluoro-7-(methoxymethoxy)-5-azaspiro[3.4]octane

To a solution of 5-(tert-butylsulfonyl)-7-(methoxymethoxy)-5-azaspiro[3.4]octan-2-ol (1 g, 3.25 mmol) in dichloromethane (10 mL) was added bis(2-methoxyethyl)aminosulfur trifluoride (1.8 g, 8.13 mmol) at 0° C. After stirred at 0° C. for 1 h, the mixture was quenched by addition of saturated aqueous sodium hydrogencarbonate (50 mL) and extracted with dichloromethane (30 mL×2). The combined organic layers were washed with brine (50 mL), dried over anhydrous sodium sulfate and concentrated under reduced pressure. The residue was purified by column chromatography to afford the title compound. ¹H NMR (400 MHz, CD₃OD) δ=5.24-5.03 (m, 1H), 4.70-4.62 (m, 2H), 4.29-4.20 (m, 1H), 3.70-3.14 (m, 7H), 2.59-2.23 (m, 4H), 1.42-1.35 (m, 9H).

### Step 8: 5-(tert-butylsulfonyl)-2-fluoro-5-azaspiro[3.4]octan-7-ol

The solution of 5-(tert-butylsulfonyl)-2-fluoro-7-(methoxymethoxy)-5-azaspiro[3.4]octane (390 mg, 1.26 mmol) in hydrochloric acid (9.45 mL, 4 M in methanol) was stirred at 20° C. for 0.5 h. The mixture was concentrated under reduced pressure to afford the title compound, which was used in next step without further purification. ¹H NMR (400 MHz, CD₃OD) δ=5.23-5.03 (m, 1H), 4.36-4.29 (m, 1H), 3.69-3.52 (m, 1H), 3.45-3.36 (m, 1H), 3.30-3.15 (m, 2H), 2.60-2.20 (m, 4H), 1.38 (s, 9H).

### Step 9: 5-(tert-butylsulfonyl)-2-fluoro-5-azaspiro[3.4]octan-7-one

To a solution of 5-(tert-butylsulfonyl)-2-fluoro-5-azaspiro[3.4]octan-7-ol (450 mg, 1.70 mmol) in dichloromethane (10 mL) was added dess-martin periodinane (1.44 g, 3.39 mmol) at 20° C. After stirred at 20° C. for 1 h, the mixture was filtered. The filtrate was quenched by addition of saturated aqueous sodium hydrogencarbonate (15 mL) and extracted with dichloromethane (15 mL×2). The combined organic layers were washed with brine (20 mL), dried over anhydrous sodium sulfate and concentrated under reduced pressure. The residue was purified by column chromatography to afford the title compound. ¹H NMR (400 MHz, CD₃OD) δ=5.33-5.02 (m, 1H), 3.84-3.77 (m, 1H), 3.66-3.45 (m, 1H), 3.29-3.12 (m, 1H), 2.94 (s, 1H), 2.55-2.30 (m, 4H), 1.39 (d, J=13.2 Hz, 9H).

### Step 10: 1-(5-(tert-butylsulfonyl)-2-fluoro-5-azaspiro[3.4]octan-7-yl)-6-chloro-1,2,3,4-tetrahydroquinoline

To a solution of 5-(tert-butylsulfonyl)-2-fluoro-5-azaspiro[3.4]octan-7-one (456 mg, 1.73 mmol) and 6-chloro-1,2,3,4-tetrahydroquinoline (290 mg, 1.73 mmol) in methanol (10 mL) and acetic acid (2 mL) was added borane-2-picoline complex (370 mg, 3.46 mmol). After stirred at 20° C. for 15 h, the mixture was concentrated under reduced pressure. The residue was diluted with saturated aqueous sodium hydrogencarbonate (50 mL) and extracted with ethyl acetate (30 mL×2). The combined organic layers were dried over anhydrous sodium sulfate and concentrated under reduced pressure. The residue was purified by column chromatography to afford the title compound. LCMS R_{T}=1.472 min, m/z=415.3 [M+H]⁺.

### Step 11: 8-bromo-1-(5-(tert-butylsulfonyl)-2-fluoro-5-azaspiro[3.4]octan-7-yl)-6-chloro-1,2,3,4-tetrahydroquinoline

To a solution of 1-(5-(tert-butylsulfonyl)-2-fluoro-5-azaspiro[3.4]octan-7-yl)-6-chloro-1,2,3,4-tetrahydroquinoline (55 mg, 0.13 mmol) in DMF (2 mL) was added 1-bromopyrrolidine-2,5-dione (24 mg, 0.13 mmol). After stirred at 20° C. for 1 h, the mixture was diluted with ethyl acetate (7 mL) and washed with water (8 mL×2). The organic layer was dried over anhydrous sodium sulfate and concentrated under reduced pressure. The residue was purified by preparative TLC to afford the title compound. LCMS R_{T}=3.148 min, m/z=493.1 [M+H]⁺.

### Step 12: 2-(((tert-butyldimethylsilyl)oxy)methyl)-7-(1-(5-(tert-butylsulfonyl)-2-fluoro-5-azaspiro[3.4]octan-7-yl)-6-chloro-1,2,3,4-tetrahydroquinolin-8-yl)thieno[3,2-b]pyridine

To a solution of 8-bromo-1-(5-(tert-butylsulfonyl)-2-fluoro-5-azaspiro[3.4]octan-7-yl)-6-chloro-1,2,3,4-tetrahydroquinoline (30 mg, 0.061 mmol) in water (0.1 mL) and dioxane (1 mL) was added (2-(((tert-butyldimethylsilyl)oxy)methyl)thieno[3,2-b]pyridin-7-yl)boronic acid (29 mg, 0.091 mmol), dichloro[1,1′-bis(diphenylphosphino)ferrocene]palladium(II) (9 mg, 0.012 mmol) and potassium carbonate (17 mg, 0.12 mmol) at 20° C. After stirred at 100° C. for 2 h under nitrogen atmosphere, the mixture was concentrated under reduced pressure to remove the solvent. The residue was purified by preparative TLC to afford the title compound. LCMS R_{T}=1.163 min, m/z=693.0 [M+H]⁺.

### Step 13: (7-(1-(5-(tert-butylsulfonyl)-2-fluoro-5-azaspiro[3.4]octan-7-yl)-6-chloro-1,2,3,4-tetrahydroquinolin-8-yl)thieno[3,2-b]pyridin-2-yl)methanol

To a solution of 2-(((tert-butyldimethylsilyl)oxy)methyl)-7-(1-(5-(tert-butylsulfonyl)-2-fluoro-5-azaspiro[3.4]octan-7-yl)-6-chloro-1,2,3,4-tetrahydroquinolin-8-yl)thieno[3,2-b]pyridine (30 mg, 0.043 mmol) in THF (1.5 mL) was added tetrabutylammonium fluoride (0.048 mL, 1 M in THF). After stirred at 20° C. for 0.5 h, the mixture was concentrated under reduced pressure. The residue was purified by preparative HPLC to afford the title compound. LCMS R_{T}=1.230 min, m/z=578.3 [M+H]⁺.

### Step 14: 1-((7-(1-(5-(tert-butylsulfonyl)-2-fluoro-5-azaspiro[3.4]octan-7-yl)-6-chloro-1,2,3,4-tetrahydroquinolin-8-yl)thieno[3,2-b]pyridin-2-yl)methyl)pyrrolidine-2,5-dione

To a solution of (7-(1-(5-(tert-butylsulfonyl)-2-fluoro-5-azaspiro[3.4]octan-7-yl)-6-chloro-1,2,3,4-tetrahydroquinolin-8-yl)thieno[3,2-b]pyridin-2-yl)methanol (10 mg, 0.017 mmol) in toluene (1.5 mL) was added succinimide (5 mg, 0.05 mmol), tetramethylazodicarboxamide (12 mg, 0.07 mmol) and tributylphosphine (21 mg, 0.10 mmol). After stirred at 100° C. for 1 h, the mixture was concentrated under reduced pressure. The residue was purified by preparative TLC to afford the title compound. LCMS R_{T}=2.423 min, m/z=659.3 [M+H]⁺.

### Step 15

A solution of 1-((7-(1-(5-(tert-butylsulfonyl)-2-fluoro-5-azaspiro[3.4]octan-7-yl)-6-chloro-1,2,3,4-tetrahydroquinolin-8-yl)thieno[3,2-b]pyridin-2-yl)methyl)pyrrolidine-2,5-dione (15 mg, 0.023 mmol) in hydrochloric acid (0.95 mL, 12 M) was stirred at 20° C. for 5 h. The mixture was concentrated under reduced pressure. The residue was purified by preparative HPLC to afford Example 211. ¹H NMR (400 MHz, CD₃OD) δ=8.63 (d, J=4.8 Hz, 1H), 8.40 (s, 1H), 7.50-7.31 (m, 2H), 7.13-7.02 (m, 2H), 4.89 (s, 2H), 3.47 (s, 1H), 3.24 (td, J=1.6, 3.2 Hz, 2H), 3.10 (s, 2H), 2.68 (s, 4H), 2.55-1.20 (m, 11H). LCMS R_{T}=1.203 min, m/z=539.4 [M+H]⁺.

### Example 212: 3-((7-(4-((R)-5,5-bis(hydroxymethyl)pyrrolidin-3-yl)-7-chloro-3,4-dihydro-2H-benzo[b][1,4]oxazin-5-yl)thieno[3,2-b]pyridin-2-yl)methyl)-3-azabicyclo[3.1.0]hexane-2,4-dione, formic acid salt and 3-((7-(4-((S)-5,5-bis(hydroxymethyl)pyrrolidin-3-yl)-7-chloro-3,4-dihydro-2H-benzo[b][1,4]oxazin-5-yl)thieno[3,2-b]pyridin-2-yl)methyl)-3-azabicyclo[3.1.0]hexane-2,4-dione, formic acid salt

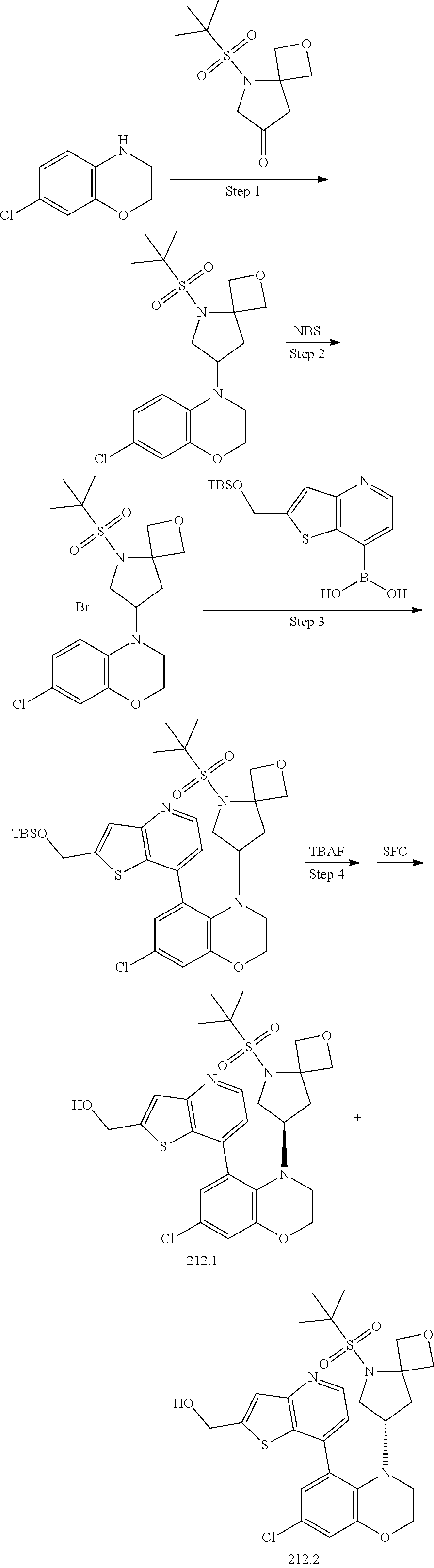

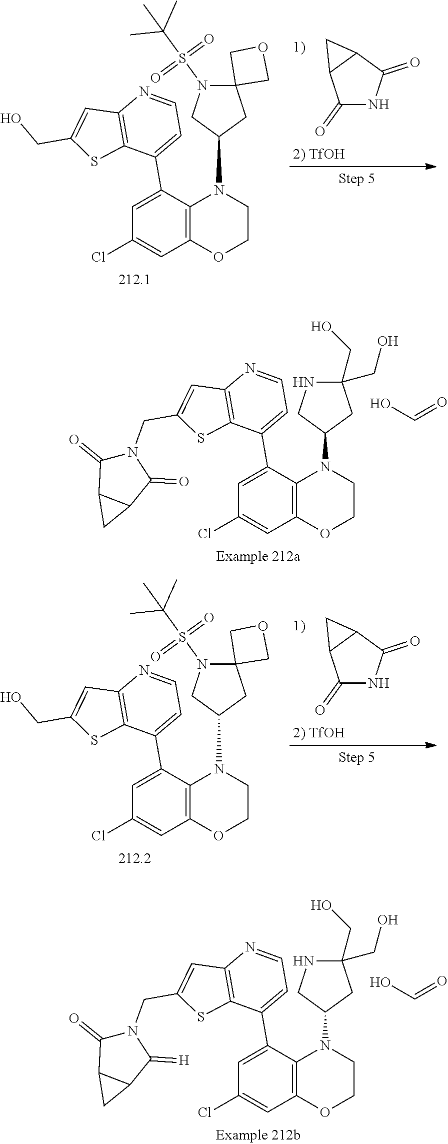

### Step 1: 4-(5-(tert-butylsulfonyl)-2-oxa-5-azaspiro[3.4]octan-7-yl)-7-chloro-3,4-dihydro-2H-benzo[b][1,4]oxazine

To a solution of 7-chloro-3,4-dihydro-2H-benzo[b][1,4]oxazine (2 g, 11.79 mmol) in acetic acid (50 mL) were added 5-(tert-butylsulfonyl)-2-oxa-5-azaspiro[3.4]octan-7-one (2.92 g, 11.79 mmol) and sodium triacetoxyborohydride (5 g, 23.58 mmol) at 25° C. After stirred at 25° C. for 12 h, the reaction mixture was concentrated under reduced pressure. The residue was diluted with aqueous sodium bicarbonate solution (50 mL) and extracted with ethyl acetate (20 mL×3). The combined organic layers were washed with brine (50 mL), dried over anhydrous sodium sulfate, filtered and the filtrate was concentrated under reduced pressure. The residue was purified by column chromatography to afford the title compound. LCMS R_{T}=1.873 min, m/z=401.2 [M+H]⁺.

### Step 2: 5-bromo-4-(5-(tert-butylsulfonyl)-2-oxa-5-azaspiro[3.4]octan-7-yl)-7-chloro-3,4-dihydro-2H-benzo[b][1,4]oxazine

To a solution of 4-(5-(tert-butylsulfonyl)-2-oxa-5-azaspiro[3.4]octan-7-yl)-7-chloro-3,4-dihydro-2H-benzo[b][1,4]oxazine (1.9 g, 4.74 mmol) in dichloromethane (25 mL) was added N-bromosuccinimide (843 mg, 4.74 mmol). After stirred at 0° C. for 1 h, the reaction mixture was concentrated under reduced pressure. Then the mixture was diluted with water (30 mL), extracted with ethyl acetate (15 mL×3). The combined organic layers were washed with brine (25 mL), dried over anhydrous sodium sulfate and concentrated under reduced pressure. The residue was purified by column chromatography to afford the title compound. LCMS R_{T}=1.738 min, m/z=481.0 [M+H]⁺.

### Step 3: 5-(2-(((tert-butyldimethylsilyl)oxy)methyl)thieno[3,2-b]pyridin-7-yl)-4-(5-(tert-butylsulfonyl)-2-oxa-5-azaspiro[3.4]octan-7-yl)-7-chloro-3,4-dihydro-2H-benzo[b][1,4]oxazine

To a solution of 5-bromo-4-(5-(tert-butylsulfonyl)-2-oxa-5-azaspiro[3.4]octan-7-yl)-7-chloro-3,4-dihydro-2H-benzo[b][1,4]oxazine (790 mg, 1.65 mmol) in dioxane (10 mL) and water (1 mL) were added potassium carbonate (455 mg, 3.29 mmol), (2-(((tert-butyldimethylsilyl)oxy)methyl)thieno[3,2-b]pyridin-7-yl)boronic acid (798 mg, 2.47 mmol) and dichloro[1,1′-bis(diphenylphosphino)ferrocene]palladium(II) (240.95 mg, 0.33 mmol). After stirred at 95° C. for 3 h, the reaction mixture was filtered and the filtrate was concentrated under reduced pressure. The residue was purified by column chromatography to afford the title compound. LCMS R_{T}=2.143 min, m/z=678.3 [M+H]⁺.

### Step 4: (R)-(7-(4-(5-(tert-butylsulfonyl)-2-oxa-5-azaspiro[3.4]octan-7-yl)-7-chloro-3,4-dihydro-2H-benzo[b][1,4]oxazin-5-yl)thieno[3,2-b]pyridin-2-yl)methanol and (S)-(7-(4-(5-(tert-butylsulfonyl)-2-oxa-5-azaspiro[3.4]octan-7-yl)-7-chloro-3,4-dihydro-2H-benzo[b][1,4]oxazin-5-yl)thieno[3,2-b]pyridin-2-yl)methanol

To a solution of 5-(2-(((tert-butyldimethylsilyl)oxy)methyl)thieno[3,2-b]pyridin-7-yl)-4-(5-(tert-butylsulfonyl)-2-oxa-5-azaspiro[3.4]octan-7-yl)-7-chloro-3,4-dihydro-2H-benzo[b][1,4]oxazine (400 mg, 0.59 mmol) in THF (5 mL) was added tetrabutylammonium fluoride (0.59 mL, 1 M in THF) at 25° C. After stirred at 25° C. for 0.5 h, the reaction mixture was diluted with water (20 mL) and extracted with dichloromethane (15 mL×3). The combined organic layers were washed with brine (25 mL), dried over anhydrous sodium sulfate, filtered and the filtrate was concentrated under reduced pressure. The residue was purified by column chromatography. The racemic product was separated by SFC to give first eluting fraction (212.1, Rt=1.967 min; LCMS R_{T}=0.493 min, m/z=564.2 [M+H]+) and second eluting fraction (212.2, Rt=2.193 min; LCMS R_{T}=0.493 min, m/z=564.2 [M+H]+)

### Step 5

To a solution of 212.1 was added 3-azabicyclo[3.1.0]hexane-2,4-dione (18 mg, 0.16 mmol), (3E)-3-(dimethylcarbamoylimino)-1,1-dimethyl-urea (27 mg, 0.16 mmol) and tributylphosphane (65 mg, 0.32 mmol) at 25° C. After stirred at 100° C. for 1 h, the reaction mixture was concentrated under reduced pressure to remove the solvent. The residue was dissolved in trifluoromethanesulfonic acid (274 mg, 1.83 mmol). After stirred at 25° C. for 1 h, the reaction mixture was concentrated under reduced pressure. The residue was purified by preparative HPLC to give Example 212a. 1H NMR (400 MHz, CD30D-d4) δ=8.74-8.65 (m, 1H), 8.56-8.47 (m, 1H), 7.47-7.39 (m, 2H), 7.05-6.86 (m, 2H), 4.80 (s, 2H), 4.31-4.02 (m, 2H), 3.43-3.36 (m, 1H), 3.38 (br d, J=11.4 Hz, 3H), 2.97-2.52 (m, 1H), 2.59 (dd, J=3.5, 8.1 Hz, 6H), 1.81-1.58 (m, 4H). LCMS R_{T}=0.787 min, m/z=555.1 [M+H]+

Example 212b was prepared from 212.2, following the procedure described in the synthesis of Example 212a. The residue was purified by preparative HPLC. ¹H NMR (400 MHz, CD₃OD) δ=8.70 (d, J=4.8 Hz, 1H), 7.48-7.38 (m, 2H), 7.01-6.89 (m, 2H), 4.80 (s, 3H), 4.36-3.55 (m, 3H), 3.51-3.33 (m, 4H), 3.26-2.72 (m, 3H), 2.67-2.50 (m, 2H), 1.93-1.02 (m, 4H). LCMS R_{T}=1.079 min, m/z=555.3 [M+H]⁺.

### Example 213a: (R)-2-((7-(4-(5,5-bis(hydroxymethyl)pyrrolidin-3-yl)-7-chloro-3,4-dihydro-2H-benzo[b][1,4]oxazin-5-yl)thieno[3,2-b]pyridin-2-yl)methyl)-5-(tert-butyl)pyridazin-3(2H)-one

Example 213a was prepared from (R)-(7-(4-(5-(tert-butylsulfonyl)-2-oxa-5-azaspiro[3.4]octan-7-yl)-7-chloro-3,4-dihydro-2H-benzo[b][1,4]oxazin-5-yl)thieno[3,2-b]pyridin-2-yl)methanol and 5-(tert-butyl)pyridazin-3(2H)-one, following the procedure described in the synthesis of Example 212a. The product was purified by preparative HPLC. ¹H NMR (400 MHz, CD₃OD-d4) 6=8.70 (d, J=4.8 Hz, 1H), 8.15 (d, J=2.4 Hz, 1H), 7.55 (s, 1H), 7.43 (d, J=4.8 Hz, 1H), 6.96-6.83 (m, 3H), 5.60 (s, 2H), 4.33-3.33 (m, 6H), 3.30-2.70 (m, 5H), 2.04 (s, 2H), 1.41-1.15 (m, 9H). LCMS R_{T}=1.747 min, m/z=596.3 [M+H]⁺

### Example 213b: (S)-2-((7-(4-(5,5-bis(hydroxymethyl)pyrrolidin-3-yl)-7-chloro-3,4-dihydro-2H-benzo[b][1,4]oxazin-5-yl)thieno[3,2-b]pyridin-2-yl)methyl)-5-(tert-butyl)pyridazin-3(2H)-one, formic acid salt

Example 213a was prepared from (S)-(7-(4-(5-(tert-butylsulfonyl)-2-oxa-5-azaspiro[3.4]octan-7-yl)-7-chloro-3,4-dihydro-2H-benzo[b][1,4]oxazin-5-yl)thieno[3,2-b]pyridin-2-yl)methanol and 4-tert-butyl-1H-pyridazin-6-one, following the procedure described in the synthesis of Example 212a. The product was purified by preparative HPLC. ¹H NMR (400 MHz, CD₃OD) δ=8.70 (d, J=4.8 Hz, 1H), 8.15 (d, J=2.4 Hz, 1H), 7.55 (s, 1H), 7.43 (d, J=4.8 Hz, 1H), 6.96-6.83 (m, 3H), 5.60 (s, 2H), 4.33-3.33 (m, 6H), 3.30-2.70 (m, 5H), 2.04 (s, 2H), 1.41-1.15 (m, 9H). LCMS R_{T}=1.404 min, m/z=596.4 [M+H]⁺.

### Example 214: 3-((7-(7-chloro-4-((3S,5R)-5-(hydroxymethyl)pyrrolidin-3-yl)-3,4-dihydro-2H-benzo[b][1,4]oxazin-5-yl)thieno[3,2-b]pyridin-2-yl)methyl)-3-azabicyclo[3.1.0]hexane-2,4-dione, formic acid salt

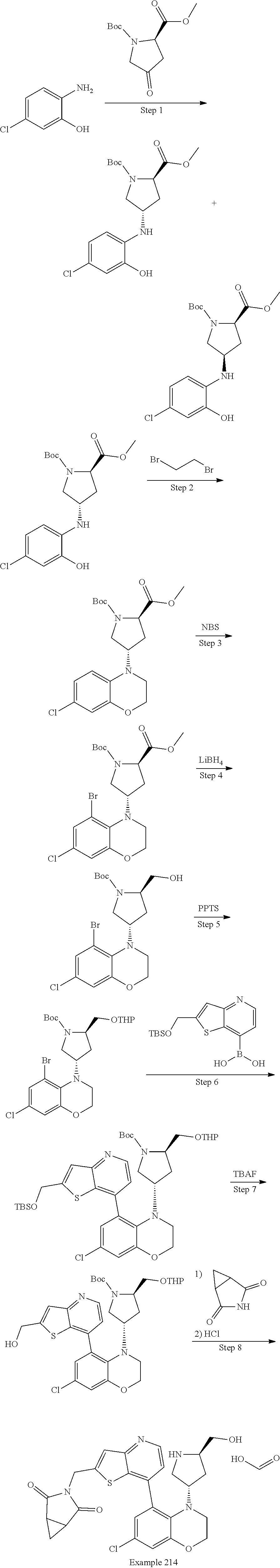

### Step 1: (2R,4S)-1-tert-butyl 2-methyl 4-((4-chloro-2-hydroxyphenyl)amino)pyrrolidine-1,2-dicarboxylate (2R,4R)-1-tert-butyl 2-methyl 4-((4-chloro-2-hydroxyphenyl)amino)pyrrolidine-1,2-dicarboxylate

To a solution of 2-amino-5-chlorophenol (7.06 g, 29.02 mmol) in dichloroethane (60 mL) was added acetic acid (1.74 g, 29.02 mmol), sodium borohydride acetate (6.15 g, 29.02 mmol) and 2-amino-5-chloro-phenol (5 g, 34.83 mmol) at 25° C. After stirred at 25° C. for 1 h, the reaction mixture was diluted with water (300 mL) and extracted with methylene chloride (10 mL×4). The combined organic layers dried over anhydrous sodium sulfate, filtered and the filtrate was concentrated under reduced pressure. The residue was purified by preparative HPLC to afford (2R,4S)-1-tert-butyl 2-methyl 4-((4-chloro-2-hydroxyphenyl)amino)pyrrolidine-1,2-dicarboxylate (Rt=2.101 min; LCMS R_{T}=1.458 min, m/z=271.2 [M+H−100]+) and (2R,4R)-1-tert-butyl 2-methyl 4-((4-chloro-2-hydroxyphenyl)amino)pyrrolidine-1,2-dicarboxylate (Rt=2.275 min; LCMS R_{T}=1.498 min, m/z=371.2 [M+H]+)

### Step 2: (2R,4S)-1-tert-butyl 2-methyl 4-(7-chloro-2H-benzo[b][1,4]oxazin-4(3H)-yl)pyrrolidine-1,2-dicarboxylate

To a solution of 1,2-dibromoethane (810 mg, 4.31 mmol) in (methylsulfinyl)methane (10 mL) was added (2R,4S)-1-tert-butyl 2-methyl 4-((4-chloro-2-hydroxyphenyl)amino)pyrrolidine-1,2-dicarboxylate (1.6 g, 4.31 mmol) and potassium carbonate (1.49 g, 10.79 mmol) at 25° C. After stirred at 80° C. for 2 h, the reaction mixture was diluted with Ammonium chloride (20 mL) and extracted with ethyl acetate (10 mL×4). The combined organic layers dried over anhydrous sodium sulfate, filtered and the filtrate was concentrated under reduced pressure. The residue was purified by column chromatography to afford the title compound. LCMS R_{T}=1.748, m/z=396.2 [M+H]⁺.

### Step 3: (2R,4S)-1-tert-butyl 2-methyl 4-(5-bromo-7-chloro-2H-benzo[b][1,4]oxazin-4(3H)-yl)pyrrolidine-1,2-dicarboxylate

To a solution of (2R,4S)-1-tert-butyl 2-methyl 4-(7-chloro-2H-benzo[b][1,4]oxazin-4(3H)-yl)pyrrolidine-1,2-dicarboxylate (890 mg, 2.24 mmol) in methylene chloride (1 mL) was added 1-bromopyrrolidine-2,5-dione (359 mg, 2.02 mmol) at 0° C. After stirred at 25° C. for 1 h, the reaction mixture was diluted with water (5 mL) and extracted with methylene chloride (5 mL×4). The combined organic layers dried over anhydrous sodium sulfate, filtered and the filtrate was concentrated under reduced pressure. The residue was purified by preparative TLC to afford the title compound. LCMS R_{T}=1.103, m/z=421.0 [M+2+H−56]⁺.

### Step 4: (2R,4S)-tert-butyl 4-(5-bromo-7-chloro-2H-benzo[b][1,4]oxazin-4(3H)-yl)-2-(hydroxymethyl)pyrrolidine-1-carboxylate

To a solution of (2R,4S)-1-tert-butyl 2-methyl 4-(5-bromo-7-chloro-2H-benzo[b][1,4]oxazin-4(3H)-yl)pyrrolidine-1,2-dicarboxylate (750 mg, 1.58 mmol) in THF (2 mL) was added lithium borohydride (140 mg, 6.46 mmol) at −78° C. After stirred at 25° C. for 12 h, the reaction mixture was diluted with ammonium chloride (20 mL) and extracted with ethyl acetate (10 mL×4). The combined organic layers dried over anhydrous sodium sulfate, filtered and the filtrate was concentrated under reduced pressure. The residue was purified by preparative TLC to afford the title compound. LCMS R_{T}=0.598, m/z=393.0 [M+2+H−56]⁺.

### Step 5: (2R,4S)-tert-butyl 4-(5-bromo-7-chloro-2H-benzo[b][1,4]oxazin-4(3H)-yl)-2-(((tetrahydro-2H-pyran-2-yl)oxy)methyl)pyrrolidine-1-carboxylate

To a solution of (2R,4S)-tert-butyl 4-(5-bromo-7-chloro-2H-benzo[b][1,4]oxazin-4(3H)-yl)-2-(hydroxymethyl)pyrrolidine-1-carboxylate (450 mg, 1.01 mmol) in acetone (5 mL) was added pyridine 4-methylbenzenesulfonate (75 mg, 0.30 mmol) and 3,4-dihydro-2H-pyran (169 mg, 2.01 mmol) at 25° C. After stirred at 45° C. for 3 h, the reaction mixture was concentrated under reduced pressure. The residue was purified by preparative TLC to afford the title compound. LCMS R_{T}=0.657, m/z=475.2 [M+H−56]⁺.

### Step 6: (2R,4S)-tert-butyl 4-(5-(2-(((tert-butyldimethylsilyl)oxy)methyl)thieno[3,2-b]pyridin-7-yl)-7-chloro-2H-benzo[b][1,4]oxazin-4(3H)-yl)-2-(((tetrahydro-2H-pyran-2-yl)oxy)methyl)pyrrolidine-1-carboxylate

To a solution of (2R,4S)-tert-butyl 4-(5-bromo-7-chloro-2H-benzo[b][1,4]oxazin-4(3H)-yl)-2-(((tetrahydro-2H-pyran-2-yl)oxy)methyl)pyrrolidine-1-carboxylate (330 mg, 0.62 mmol) in dioxane (5 mL)/water (0.5 mL) was added [2-[[tert-butyl(dimethyl)silyl]oxymethyl]thieno[3,2-b]pyridin-7-yl]boronic acid (200 mg, 0.62 mmol), palladium dichloride[1,1′-bis (diphenylphosphine) ferrocene] (45 mg, 0.06 mmol) and cesium carbonate (404 mg, 1.24 mmol) at 25° C. After stirred at 100° C. for 2 h, the reaction mixture was concentrated under reduced pressure. The residue was purified by column chromatography to afford the title compound. LCMS R_{T}=0.702, m/z=730.3 [M+H]⁺.

### Step 7: (2R,4S)-tert-butyl 4-(7-chloro-5-(2-(hydroxymethyl)thieno[3,2-b]pyridin-7-yl)-2H-benzo[b][1,4]oxazin-4(3H)-yl)-2-(((tetrahydro-2H-pyran-2-yl)oxy)methyl)pyrrolidine-1-carboxylate

To a solution of (2R,4S)-tert-butyl 4-(5-(2-(((tert-butyldimethylsilyl)oxy)methyl)thieno[3,2-b]pyridin-7-yl)-7-chloro-2H-benzo[b][1,4]oxazin-4(3H)-yl)-2-(((tetrahydro-2H-pyran-2-yl)oxy)methyl)pyrrolidine-1-carboxylate (200 mg, 0.28 mmol) in THF (2 mL) was added tetrabutylammonium fluoride (1 M, 0.3 mL) at 25° C. After stirred at 25° C. for 0.5 h, the reaction mixture was concentrated under reduced pressure. The residue was purified by column chromatography to afford the title compound. LCMS R_{T}=1.298, m/z=616.3 [M+H]⁺.

### Step 8

Example 214 was prepared from (2R,4S)-tert-butyl 4-(7-chloro-5-(2-(hydroxymethyl)thieno[3,2-b]pyridin-7-yl)-2H-benzo[b][1,4]oxazin-4(3H)-yl)-2-(((tetrahydro-2H-pyran-2-yl)oxy)methyl)pyrrolidine-1-carboxylate and 3-azabicyclo[3.1.0]hexane-2,4-dione, following the procedure described in the synthesis of Example 108. The final compound was purified by preparative HPLC. ¹H NMR (400 MHz, CD₃OD) δ=8.67-8.72 (m, 1H), 8.49 (s, 1H), 7.41-7.46 (m, 2H), 6.99 (d, J=2.40 Hz, 1H), 6.92 (d, J=2.40 Hz, 1H), 4.80 (s, 2H), 4.11-4.27 (m, 2H), 3.60-3.72 (m, 1H), 3.49 (d, J=2.0 Hz, 1H), 3.36-3.42 (m, 1H), 3.36-3.76 (m, 1H), 2.99-3.13 (m, 1H), 2.67-2.80 (m, 1H), 2.66-3.17 (m, 1H), 2.59 (dd, J=8.00, 3.6 Hz, 2H), 1.74-1.94 (m, 1H), 1.62 (td, J=8.00, 4.64 Hz, 2H), 1.41 (q, J=3.6 Hz, 2H). LCMS R_{T}=1.120, m/z=525.3 [M+H]⁺.

### Example 215: 5-(2-((2,4-dioxo-3-azabicyclo[3.1.0]hexan-3-yl)methyl)thieno[3,2-b]pyridin-7-yl)-4-((3S,5R)-5-(hydroxymethyl)pyrrolidin-3-yl)-3,4-dihydro-2H-benzo[b][1,4]oxazine-7-carbonitrile, formic acid salt

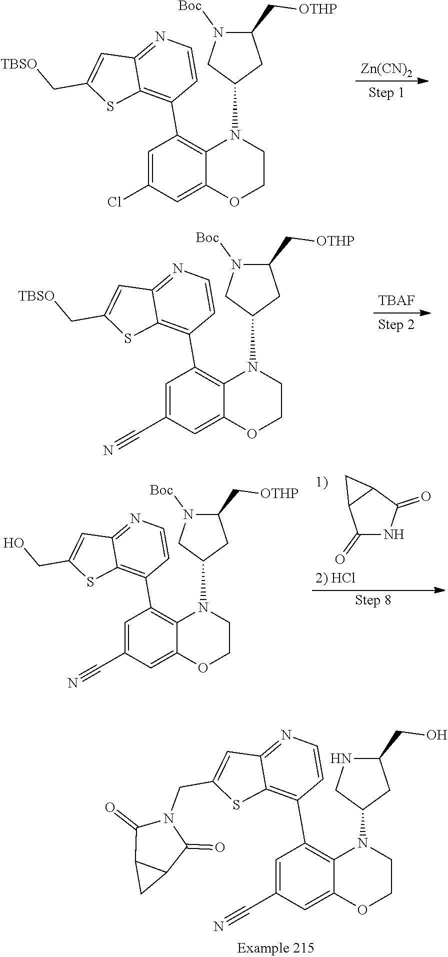

### Step 1: (2R,4S)-tert-butyl 4-(5-(2-(((tert-butyldimethylsilyl)oxy)methyl)thieno[3,2-b]pyridin-7-yl)-7-cyano-2H-benzo[b][1,4]oxazin-4(3H)-yl)-2-(((tetrahydro-2H-pyran-2-yl)oxy)methyl)pyrrolidine-1-carboxylate

To a solution of (2R,4S)-tert-butyl 4-(5-(2-(((tert-butyldimethylsilyl)oxy)methyl)thieno[3,2-b]pyridin-7-yl)-7-chloro-2H-benzo[b][1,4]oxazin-4(3H)-yl)-2-(((tetrahydro-2H-pyran-2-yl)oxy)methyl)pyrrolidine-1-carboxylate (160 mg, 0.22 mmol) in 1-methylpyrrolidin-2-one (3 mL) was added palladium; tritert-butylphosphane (33 mg, 0.06 mmol) and cyanide zinc (205 mg, 1.75 mmol) at 25° C. with microwave. After stirred at 165° C. for 1.5 h, the reaction mixture was diluted with water (10 mL) and extracted with ethyl acetate (5 mL×4). The combined organic layers dried over anhydrous sodium sulfate, filtered and the filtrate was concentrated under reduced pressure. The residue was purified by preparative TLC to afford the title compound. LCMS R_{T}=1.150, m/z=721.4 [M+H]⁺.

### Step 2: (2R,4S)-tert-butyl 4-(7-cyano-5-(2-(hydroxymethyl)thieno[3,2-b]pyridin-7-yl)-2H-benzo[b][1,4]oxazin-4(3H)-yl)-2-(((tetrahydro-2H-pyran-2-yl)oxy)methyl)pyrrolidine-1-carboxylate

To a solution of (2R,4S)-tert-butyl 4-(5-(2-(((tert-butyldimethylsilyl)oxy)methyl)thieno[3,2-b]pyridin-7-yl)-7-cyano-2H-benzo[b][1,4]oxazin-4(3H)-yl)-2-(((tetrahydro-2H-pyran-2-yl)oxy)methyl)pyrrolidine-1-carboxylate (50 mg, 0.07 mmol) in THF (1 mL) was added tetrabutylammonium fluoride (1 M, 0.07 mL) at 25° C. After stirred at 25° C. for 0.5 h, the reaction mixture was diluted with water (5 mL) and extracted with ethyl acetate (5 mL×4). The combined organic layers dried over anhydrous sodium sulfate, filtered and the filtrate was concentrated under reduced pressure. The residue was purified by preparative TLC to afford the title compound. LCMS R_{T}=1.130, m/z=607.3 [M+H]⁺.

### Step 3

Example 215 was prepared from (2R,4S)-tert-butyl 4-(7-cyano-5-(2-(hydroxymethyl)thieno[3,2-b]pyridin-7-yl)-2H-benzo[b][1,4]oxazin-4(3H)-yl)-2-(((tetrahydro-2H-pyran-2-yl)oxy)methyl)pyrrolidine-1- and 3-azabicyclo[3.1.0]hexane-2,4-dione, following the procedure described in the synthesis of Example 108. The final compound was purified by preparative HPLC. ¹H NMR (400 MHz, CD₃OD) δ=8.68-8.76 (m, 1H), 8.48 (s, 1H), 7.43-7.50 (m, 2H), 7.21-7.29 (m, 2H), 4.80 (s, 2H), 4.06-4.41 (m, 2H), 3.69-3.96 (m, 1H), 3.31-3.66 (m, 5H), 2.82-3.24 (m, 2H), 2.59 (dd, J=8.12, 3.52 Hz, 2H), 1.72-2.07 (m, 1H), 1.52-1.70 (m, 2H), 1.41 (q, J=3.64 Hz, 1H). LCMS R_{T}=0.840, m/z=516.4 [M+H]⁺.

### Example 216: 2-((7-(7-chloro-4-((3S,5R)-5-(hydroxymethyl)pyrrolidin-3-yl)-3,4-dihydro-2H-benzo[b][1,4]oxazin-5-yl)thieno[3,2-b]pyridin-2-yl)methyl)-5-methylpyridazin-3(2H)-one, formic acid salt

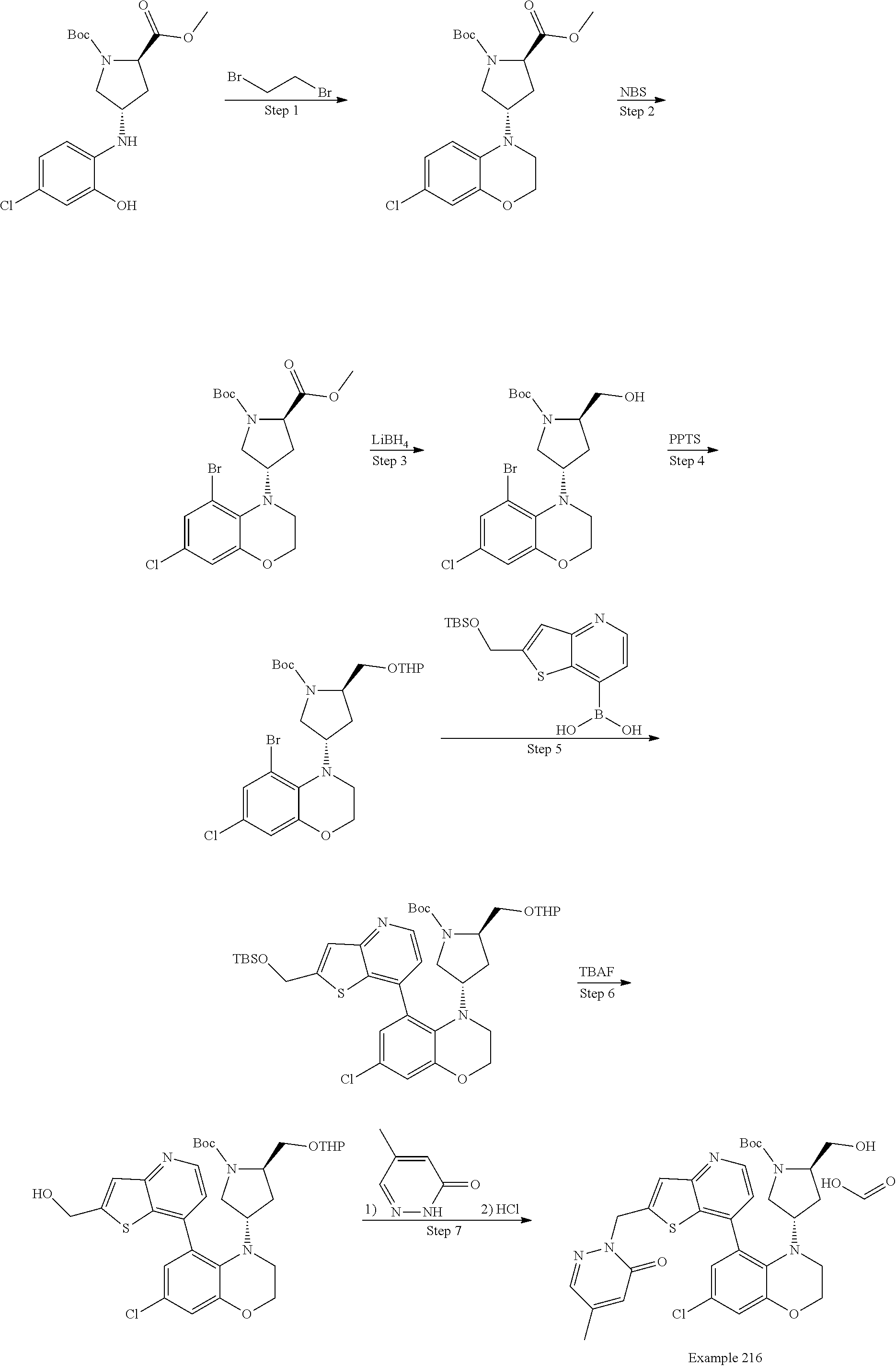

### Step 1: (2R,4S)-1-tert-butyl 2-methyl 4-(7-chloro-2H-benzo[b][1,4]oxazin-4(3H)-yl)pyrrolidine-1,2-dicarboxylate

To a solution of (2R,4S)-1-tert-butyl 2-methyl 4-((4-chloro-2-hydroxyphenyl)amino)pyrrolidine-1,2-dicarboxylate (7.1 g, 19.15 mmol) in (methylsulfinyl)methane (35.5 mL) was added 1,2-dibromoethane (14.39 g, 76.59 mmol) and potassium carbonate (6.62 g, 47.87 mmol) in (methylsulfinyl)methane (35 mL) at 80° C. with peristaltic pump. After stirred at 80° C. for 12 h, the reaction mixture was diluted with ammonium chloride (100 mL) and extracted with ethyl acetate (30 mL×4). The combined organic layers dried over anhydrous sodium sulfate, filtered and the filtrate was concentrated under reduced pressure. The residue was purified by column chromatography to afford the title compound. LCMS R_{T}=1.033, m/z=397.1 [M+H]⁺.

### Step 2: (2R,4S)-1-tert-butyl 2-methyl 4-(5-bromo-7-chloro-2H-benzo[b][1,4]oxazin-4(3H)-yl)pyrrolidine-1,2-dicarboxylate

To a solution of (2R,4S)-1-tert-butyl 2-methyl 4-(7-chloro-2H-benzo[b][1,4]oxazin-4(3H)-yl)pyrrolidine-1,2-dicarboxylate (3.2 g, 8.06 mmol) in methylene chloride (2 mL) was added 1-bromopyrrolidine-2,5-dione (1.65 g, 9.27 mmol) at 0° C. After stirred at 25° C. for 1 h, the reaction mixture was diluted with water (30 mL) and extracted with methylene chloride (10 mL×4). The combined organic layers dried over anhydrous sodium sulfate, filtered and the filtrate was concentrated under reduced pressure. The residue was purified by column chromatography to afford the title compound. LCMS R_{T}=1.100, m/z=476.8 [M+1+H].

### Step 3: (2R,4S)-tert-butyl 4-(5-bromo-7-chloro-2H-benzo[b][1,4]oxazin-4(3H)-yl)-2-(hydroxymethyl)pyrrolidine-1-carboxylate

To a solution of (2R,4S)-1-tert-butyl 2-methyl 4-(5-bromo-7-chloro-2H-benzo[b][1,4]oxazin-4(3H)-yl)pyrrolidine-1,2-dicarboxylate (3.2 g, 6.73 mmol) in THF (20 mL) was added lithium borohydride (360 mg, 16.55 mmol,) at 0° C. After stirred at 80° C. for 12 h, the reaction mixture was diluted with ammonium chloride (100 mL) and extracted with ethyl acetate (30 mL×4). The combined organic layers dried over anhydrous sodium sulfate, filtered and the filtrate was concentrated under reduced pressure. The residue was purified by column chromatography to afford the title compound. LCMS R_{T}=1.040, m/z=449.0 [M+2+H]⁺.

### Step 4: (2R,4S)-tert-butyl 4-(5-bromo-7-chloro-2H-benzo[b][1,4]oxazin-4(3H)-yl)-2-(((tetrahydro-2H-pyran-2-yl)oxy)methyl)pyrrolidine-1-carboxylate

To a solution of (2R,4S)-tert-butyl 4-(5-bromo-7-chloro-2H-benzo[b][1,4]oxazin-4(3H)-yl)-2-(hydroxymethyl)pyrrolidine-1-carboxylate (1.9 g, 4.24 mmol) in acetone (5 mL) was added pyridine 4-methylbenzenesulfonate (106 mg, 0.42 mmol) and 3,4-dihydro-2H-pyran (178 mg, 2.12 mmol) at 25° C. After stirred at 45° C. for 3 h, the reaction mixture was concentrated under reduced pressure. The residue was purified by column chromatography to afford the title compound. LCMS R_{T}=2.082, m/z=533.2 [M+2+H]⁺.

### Step 5: (2R,4S)-tert-butyl 4-(5-(2-(((tert-butyldimethylsilyl)oxy)methyl)thieno[3,2-b]pyridin-7-yl)-7-chloro-2H-benzo[b][1,4]oxazin-4(3H)-yl)-2-(((tetrahydro-2H-pyran-2-yl)oxy)methyl)pyrrolidine-1-carboxylate

To a solution of (2R,4S)-tert-butyl 4-(5-bromo-7-chloro-2H-benzo[b][1,4]oxazin-4(3H)-yl)-2-(((tetrahydro-2H-pyran-2-yl)oxy)methyl)pyrrolidine-1-carboxylate (1.9 g, 3.57 mmol) in dioxane (20 mL)/water (2 mL) was added [2-[[tert-butyl(dimethyl)silyl]oxymethyl]thieno[3,2-b]pyridin-7-yl]boronic acid (923 mg, 2.86 mmol), palladium dichloride[1,1′-bis (diphenylphosphine) ferrocene] (261 mg, 0.36 mmol) and cesium carbonate (2.33 g, 7.14 mmol) at 25° C. After stirred at 100° C. for 3 h, the reaction mixture was concentrated under reduced pressure. The residue was purified by column chromatography to afford the title compound. LCMS R_{T}=0.677, m/z=730.5 [M+H]⁺.

### Step 6: (2R,4S)-tert-butyl 4-(7-chloro-5-(2-(hydroxymethyl)thieno[3,2-b]pyridin-7-yl)-2H-benzo[b][1,4]oxazin-4(3H)-yl)-2-(((tetrahydro-2H-pyran-2-yl)oxy)methyl)pyrrolidine-1-carboxylate

To a solution of (2R,4S)-tert-butyl 4-(5-(2-(((tert-butyldimethylsilyl)oxy)methyl)thieno[3,2-b]pyridin-7-yl)-7-chloro-2H-benzo[b][1,4]oxazin-4(3H)-yl)-2-(((tetrahydro-2H-pyran-2-yl)oxy)methyl)pyrrolidine-1-carboxylate (840 mg, 1.15 mmol) in THF (2 mL) was added tetrabutylammonium fluoride (1 M, 1.2 mL) at 25° C. After stirred at 25° C. for 0.5 h, the reaction mixture was diluted with water (20 mL) and extracted with ethyl acetate (15 mL×4). The combined organic layers dried over anhydrous sodium sulfate, filtered and the filtrate was concentrated under reduced pressure. The residue was purified by column chromatography to afford the title compound. LCMS R_{T}=0.552, m/z=616.2 [M+H]⁺.

### Step 7

Example 216 was prepared from (2R,4S)-tert-butyl 4-(7-chloro-5-(2-(hydroxymethyl)thieno[3,2-b]pyridin-7-yl)-2H-benzo[b][1,4]oxazin-4(3H)-yl)-2-(((tetrahydro-2H-pyran-2-yl)oxy)methyl)pyrrolidine-1-carboxylate and 4-methyl-1H-pyridazin-6-one, following the procedure described in the synthesis of Example 108. The final compound was purified by preparative HPLC. ¹H NMR (400 MHz, CD₃OD) δ=8.67-8.73 (m, 1H), 8.50 (s, 1H), 7.87 (d, J=2.00 Hz, 1H), 7.54 (s, 1H), 7.39-7.45 (m, 1H), 6.97 (d, J=2.40 Hz, 1H), 6.89 (d, J=2.40 Hz, 1H), 6.80 (d, J=0.8 Hz, 1H), 5.59 (s, 2H), 4.05-4.29 (m, 2H), 3.65 (d, J=2.00 Hz, 1H), 3.32-3.53 (m, 3H), 2.97-3.15 (m, 1H), 2.59-2.82 (m, 1H), 2.48-3.17 (m, 2H), 2.25 (d, J=1.20 Hz, 3H) 2.04 (s, 2H). LCMS R_{T}=1.400, m/z=524.3 [M+H]⁺.

### Example 217: 5-(2-((4-(tert-butyl)-6-oxopyridazin-1(6H)-yl)methyl)thieno[3,2-b]pyridin-7-yl)-4-((3S,5R)-5-(hydroxymethyl)pyrrolidin-3-yl)-3,4-dihydro-2H-benzo[b][1,4]oxazine-7-carbonitrile, formic acid salt

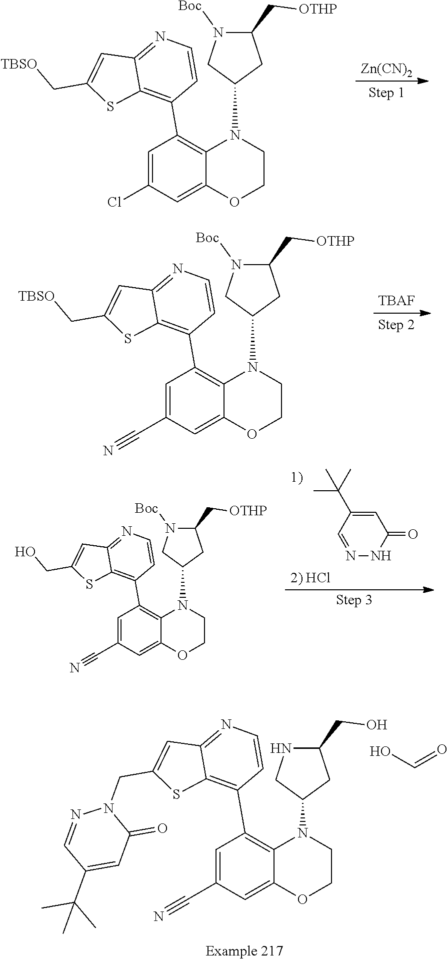

### Step 1: (2R,4S)-tert-butyl 4-(5-(2-(((tert-butyldimethylsilyl)oxy)methyl)thieno[3,2-b]pyridin-7-yl)-7-cyano-2H-benzo[b][1,4]oxazin-4(3H)-yl)-2-(((tetrahydro-2H-pyran-2-yl)oxy)methyl)pyrrolidine-1-carboxylate

To a solution of (2R,4S)-tert-butyl 4-(5-(2-(((tert-butyldimethylsilyl)oxy)methyl)thieno[3,2-b]pyridin-7-yl)-7-chloro-2H-benzo[b][1,4]oxazin-4(3H)-yl)-2-(((tetrahydro-2H-pyran-2-yl)oxy)methyl)pyrrolidine-1-carboxylate (800 mg, 1.10 mmol) in 1-methylpyrrolidin-2-one (8 mL) was added palladium; tritert-butylphosphane (167 mg, 0.33 mmol) and cyanide zinc (353 mg, 3.01 mmol) at 25° C. with microwave. After stirred at 165° C. for 1.5 h, the reaction mixture was diluted with water (10 mL) and extracted with Ethyl acetate (10 mL×4). The combined organic layers dried over anhydrous sodium sulfate, filtered and the filtrate was concentrated under reduced pressure. The residue was purified by column chromatography to afford the title compound. LCMS R_{T}=1.160, m/z=721.3 [M+H]⁺.

### Step 2: (2R,4S)-tert-butyl 4-(7-cyano-5-(2-(hydroxymethyl)thieno[3,2-b]pyridin-7-yl)-2H-benzo[b][1,4]oxazin-4(3H)-yl)-2-(((tetrahydro-2H-pyran-2-yl)oxy)methyl)pyrrolidine-1-carboxylate

To a solution of (2R,4S)-tert-butyl 4-(5-(2-(((tert-butyldimethylsilyl)oxy)methyl)thieno[3,2-b]pyridin-7-yl)-7-cyano-2H-benzo[b][1,4]oxazin-4(3H)-yl)-2-(((tetrahydro-2H-pyran-2-yl)oxy)methyl)pyrrolidine-1-carboxylate (700 mg, 0.97 mmol) in THF (5 mL) was added tetrabutylammonium fluoride (1 M, 1 mL) at 25° C. After stirred at 25° C. for 0.5 h, the reaction mixture was diluted with water (20 mL) and extracted with ethyl acetate (15 mL×4). The combined organic layers dried over anhydrous sodium sulfate, filtered and the filtrate was concentrated under reduced pressure. The residue was purified by column chromatography to afford the title compound. LCMS R_{T}=0.515, m/z=607.3 [M+H]⁺.

### Step 3

Example 217 was prepared from (2R,4S)-tert-butyl 4-(7-cyano-5-(2-(hydroxymethyl)thieno[3,2-b]pyridin-7-yl)-2H-benzo[b][1,4]oxazin-4(3H)-yl)-2-(((tetrahydro-2H-pyran-2-yl)oxy)methyl)pyrrolidine-1-carboxylate and 5-tert-butyl-4H-pyridazin-3-one, following the procedure described in the synthesis of Example 108. The final compound was purified by preparative HPLC. ¹H NMR (400 MHz, CD₃OD) δ=8.90-8.98 (m, 1H), 8.17 (d, J=2.4 Hz, 1H), 7.91 (d, J=5.6 Hz, 1H), 7.80 (s, 1H), 7.33-7.41 (m, 2H), 6.86 (d, J=2.4 Hz, 1H), 5.71 (s, 2H), 4.08-4.40 (m, 2H), 3.32-3.93 (m, 6H), 2.71-3.17 (m, 2H), 2.00-2.20 (m, 1H), 1.57-1.88 (m, 1H), 1.29-1.32 (m, 9H). LCMS R_{T}=1.467, m/z=557.5 [M+H]⁺.

### Example 218: 5-(2-((2,4-dioxo-3-azabicyclo[3.1.0]hexan-3-yl)methyl)thieno[3,2-b]pyridin-7-yl)-4-((3S,5R)-5-(hydroxymethyl)-5-methylpyrrolidin-3-yl)-3,4-dihydro-2H-benzo[b][1,4]oxazine-7-carbonitrile, formic acid salt

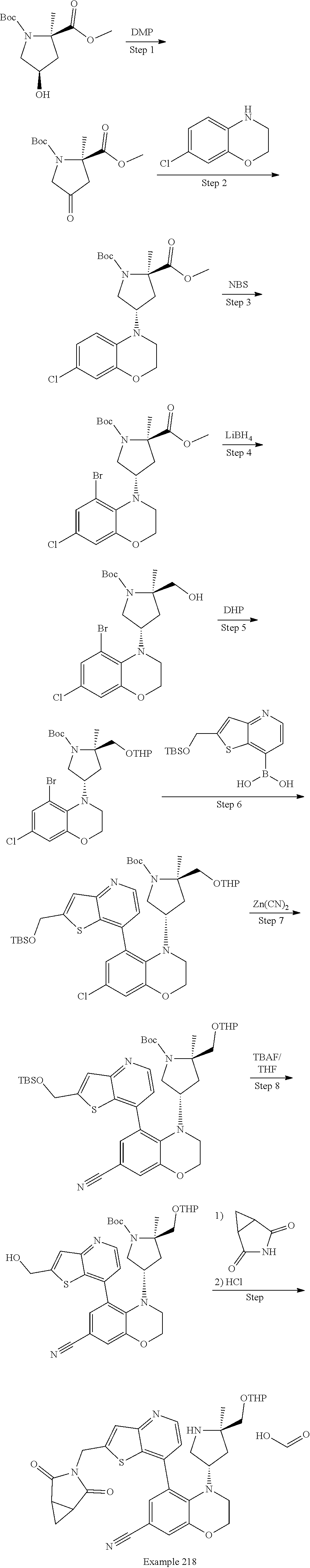

### Step 1: (R)-1-tert-butyl 2-methyl 2-methyl-4-oxopyrrolidine-1,2-dicarboxylate

To a solution of (2R,4R)-1-tert-butyl 2-methyl 4-hydroxy-2-methylpyrrolidine-1,2-dicarboxylate (6.5 g, 25.07 mmol) in dichloromethane (100 mL) was added Dess-Martin periodinane (15.95 g, 37.60 mmol). After stirred at 15° C. for 4 h, the mixture was quenched by addition of water (100 mL) and filtered. The filtrate was extracted with dichloromethane (100 mL). The organic layer was washed with brine (100 mL), dried over anhydrous sodium sulfate and concentrated under reduced pressure. The residue was purified by column chromatography to afford the title compound.

### Step 2: (2R,4S)-1-tert-butyl 2-methyl 4-(7-chloro-2H-benzo[b][1,4]oxazin-4(3H)-yl)-2-methylpyrrolidine-1,2-dicarboxylate

To a solution of (R)-1-tert-butyl 2-methyl 2-methyl-4-oxopyrrolidine-1,2-dicarboxylate (2.8 g, 10.88 mmol) and 7-chloro-3,4-dihydro-2H-benzo[b][1,4]oxazine (1.94 g, 11.43 mmol) in acetic acid (40 mL) was added sodium triacetoxyborohydride (4.61 g, 21.77 mmol). After stirred at 25° C. for 12 h, the mixture was diluted with ethyl acetate (100 mL) and washed with hydrochloric acid (3 M, 30 mL×5). The organic layer was washed with brine (100 mL), dried over anhydrous sodium sulfate and concentrated under reduced pressure. The residue was purified by column chromatography to afford the title compound. LCMS R_{T}=0.615 min, m/z=411.1 [M+H]⁺.

### Step 3: (2R,4S)-1-tert-butyl 2-methyl 4-(5-bromo-7-chloro-2H-benzo[b][1,4]oxazin-4(3H)-yl)-2-methylpyrrolidine-1,2-dicarboxylate

To a solution of (2R,4S)-1-tert-butyl 2-methyl 4-(7-chloro-2H-benzo[b][1,4]oxazin-4(3H)-yl)-2-methylpyrrolidine-1,2-dicarboxylate (400 mg, 0.97 mmol) in DMF (5 mL) was added N-bromosuccinimide (182 mg, 1.02 mmol). After stirred at 15° C. for 0.5 h, the mixture was diluted ethyl acetate (20 mL). The organic layer was washed with brine (30 mL×2), dried over anhydrous sodium sulfate and concentrated under reduced pressure. The residue was purified by column chromatography to afford the title compound. LCMS R_{T}=1.139 min, m/z=491.0 [M+2+H]⁺.

### Step 4: (2R,4S)-tert-butyl 4-(5-bromo-7-chloro-2H-benzo[b][1,4]oxazin-4(3H)-yl)-2-(hydroxymethyl)-2-methylpyrrolidine-1-carboxylate

To a solution of (2R,4S)-1-tert-butyl 2-methyl 4-(5-bromo-7-chloro-2H-benzo[b][1,4]oxazin-4(3H)-yl)-2-methylpyrrolidine-1,2-dicarboxylate (260 mg, 0.53 mmol) in THF (2.5 mL) was added lithium borohydride (80 mg, 3.67 mmol) at −65° C. After stirred at 25° C. for 12 h, the mixture was quenched by addition of saturated ammonium chloride (30 mL) and extracted with ethyl acetate (20 mL×3). The combined organic layers were washed with brine (30 mL), dried over anhydrous sodium sulfate and concentrated under reduced pressure. The residue was purified by column chromatography to afford the title compound. LCMS R_{T}=1.063 min, m/z=463.0 [M+2+H]⁺.

### Step 5: (2R,4S)-tert-butyl 4-(5-bromo-7-chloro-2H-benzo[b][1,4]oxazin-4(3H)-yl)-2-methyl-2-(((tetrahydro-2H-pyran-2-yl)oxy)methyl)pyrrolidine-1-carboxylate

To a solution of (2R,4S)-tert-butyl 4-(5-bromo-7-chloro-2H-benzo[b][1,4]oxazin-4(3H)-yl)-2-(hydroxymethyl)-2-methylpyrrolidine-1-carboxylate (140 mg, 0.30 mmol) in acetone (2 mL) was added (3,4-dihydro-2H-pyran-2-yl)methanol (51 mg, 0.61 mmol) and pyridinium p-toluenesulfonate (30 mg, 0.12 mmol). After stirred at 45° C. for 4 h, the mixture was quenched by addition of saturated sodium bicarbonate (20 mL) and extracted with ethyl acetate (15 mL×2). The combined organic layers were washed with brine (20 mL), dried over anhydrous sodium sulfate and concentrated under reduced pressure. The residue was purified by preparative TLC to afford the title compound. LCMS R_{T}=2.120 min, m/z=547.1 [M+2+H]⁺.

### Step 6: (2R,4S)-tert-butyl 4-(5-(2-(((tert-butyldimethylsilyl)oxy)methyl)thieno[3,2-b]pyridin-7-yl)-7-chloro-2H-benzo[b][1,4]oxazin-4(3H)-yl)-2-methyl-2-(((tetrahydro-2H-pyran-2-yl)oxy)methyl)pyrrolidine-1-carboxylate

To a solution of (2R,4S)-tert-butyl 4-(5-bromo-7-chloro-2H-benzo[b][1,4]oxazin-4(3H)-yl)-2-methyl-2-(((tetrahydro-2H-pyran-2-yl)oxy)methyl)pyrrolidine-1-carboxylate (150 mg, 0.27 mmol) in dioxane (2.5 mL) and water (0.2 mL) was added potassium carbonate (76 mg, 0.55 mmol), [2-[[tert-butyl(dimethyl)silyl]oxymethyl]thieno[3,2-b]pyridin-7-yl]boronic acid (107 mg, 0.33 mmol) and dichloro[1,1′-bis(diphenylphosphino)ferrocene]palladium(II) (30 mg, 0.04 mmol). After stirred at 85° C. for 3 h, the mixture was filtered and the filtrate was concentrated under reduced pressure. The residue was purified by column chromatography to afford the title compound. LCMS R_{T}=1.815 min, m/z=744.4 [M+H]⁺.

### Step 7: (2R,4S)-tert-butyl 4-(5-(2-(((tert-butyldimethylsilyl)oxy)methyl)thieno[3,2-b]pyridin-7-yl)-7-cyano-2H-benzo[b][1,4]oxazin-4(3H)-yl)-2-methyl-2-(((tetrahydro-2H-pyran-2-yl)oxy)methyl)pyrrolidine-1-carboxylate

To a solution of (2R,4S)-tert-butyl 4-(5-(2-(((tert-butyldimethylsilyl)oxy)methyl)thieno[3,2-b]pyridin-7-yl)-7-chloro-2H-benzo[b][1,4]oxazin-4(3H)-yl)-2-methyl-2-(((tetrahydro-2H-pyran-2-yl)oxy)methyl)pyrrolidine-1-carboxylate (150 mg, 0.20 mmol) in 1-methylpyrrolidin-2-one (2 mL) was added zinc cyanide (200 mg, 1.70 mmol) and bis(tri-tert-butylphosphine) palladium(0) (15 mg, 0.03 mmol). After stirred at 160° C. for 2 h under microwave, the mixture was diluted with water (10 mL) and extracted with ethyl acetate (10 mL×2). The combined organic layers were washed with brine (10 mL), dried over anhydrous sodium sulfate, filtered and the filtrate was concentrated under reduced pressure. The residue was purified by preparative TLC to afford the title compound. LCMS R_{T}=1.495 min, m/z=735.5 [M+H]⁺.

### Step 8: (2R,4S)-tert-butyl 4-(7-cyano-5-(2-(hydroxymethyl)thieno[3,2-b]pyridin-7-yl)-2H-benzo[b][1,4]oxazin-4(3H)-yl)-2-methyl-2-(((tetrahydro-2H-pyran-2-yl)oxy)methyl)pyrrolidine-1-carboxylate

To a solution of (2R,4S)-tert-butyl 4-(5-(2-(((tert-butyldimethylsilyl)oxy)methyl)thieno[3,2-b]pyridin-7-yl)-7-cyano-2H-benzo[b][1,4]oxazin-4(3H)-yl)-2-methyl-2-(((tetrahydro-2H-pyran-2-yl)oxy)methyl)pyrrolidine-1-carboxylate (90 mg, 0.12 mmol) in THF (1.5 mL) was added tetrabutylammonium fluoride (0.1 mL, 1.0 M in THF). After stirred for 0.5 h at 20° C., the residue was concentrated under reduced pressure. The residue was purified by column chromatography to afford the title compound. LCMS R_{T}=0.913 min, m/z=621.3 [M+H]⁺.

### Step 9

Example 218 was prepared from (2R,4S)-tert-butyl 4-(7-cyano-5-(2-(hydroxymethyl)thieno[3,2-b]pyridin-7-yl)-2H-benzo[b][1,4]oxazin-4(3H)-yl)-2-methyl-2-(((tetrahydro-2H-pyran-2-yl)oxy)methyl)pyrrolidine-1-carboxylate and 3-azabicyclo[3.1.0]hexane-2,4-dione, following the procedure described in the synthesis of Example 108. The final product was purified by preparative HPLC. ¹H NMR (400 MHz, CD₃DO) 6=8.72 (d, J=4.8 Hz, 1H), 8.52 (s, 1H), 7.51-7.42 (m, 2H), 7.30-7.21 (m, 2H), 4.80 (s, 1H), 4.79-4.76 (m, 1H), 4.46-4.05 (m, 2H), 3.98-3.62 (m, 1H), 3.48-3.31 (m, 3H), 3.11-2.71 (m, 3H), 2.58 (dd, J=3.6, 8.0 Hz, 2H), 1.77-1.30 (m, 4H), 1.18 (s, 3H). LCMS R_{T}=1.290 min, m/z=530.4 [M+H]⁺.

### Example 219: 5-(2-((4-(dimethylamino)-3-ethyl-2,6-dioxo-2,3-dihydropyrimidin-1(6H)-yl)methyl)thieno[3,2-b]pyridin-7-yl)-4-((3R,5S)-5-(hydroxymethyl)-5-methylpyrrolidin-3-yl)-3,4-dihydro-2H-benzo[b][1,4]oxazine-7-carbonitrile

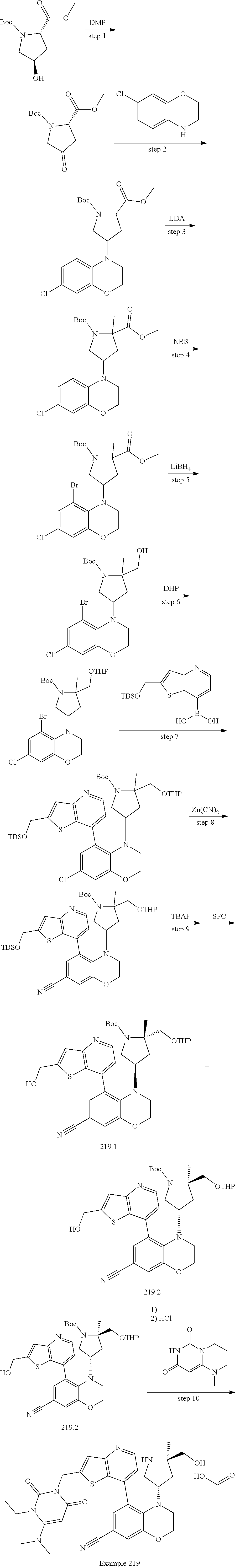

### Step 1: (S)-1-tert-butyl 2-methyl 4-oxopyrrolidine-1,2-dicarboxylate

To a solution of (2S,4R)-1-tert-butyl 2-methyl 4-hydroxypyrrolidine-1,2-dicarboxylate (50 g, 204 mmol) in dichloromethane (500 mL) was added dess martin periodide (104 g, 254 mmol) at 0° C. After stirred at 20° C. for 16 h, the reaction mixture was quenched by addition of sodium bicarbonate (100 mL) and extracted with dichloromethane (300 mL). The combined organic layers were washed with brine (300 mL), dried over anhydrous sodium sulfate, filtered and the filtrate was concentrated under reduced pressure. The residue was purified by column chromatography to afford the title compound.

### Step 2: 1-tert-butyl 2-methyl 4-(7-chloro-2H-benzo[b][1,4]oxazin-4(3H)-yl)pyrrolidine-1,2-dicarboxylate

To a solution of (S)-1-tert-butyl 2-methyl 4-oxopyrrolidine-1,2-dicarboxylate (58 g, 240 mmol) and 7-chloro-3,4-dihydro-2H-1,4-benzoxazine (40 g, 235.84 mmol) in acetonitrile (300 mL) was added trifluoroacetic acid (134.5 g, 1.18 mmol) and sodium borohydride acetate (100 g, 471.68 mmol). After stirred at 20° C. for 1.5 h, the reaction mixture was quenched by addition of sodium bicarbonate solution (200 mL) and extracted with ethyl acetate (300 mL). The combined organic layers were washed with brine (300 mL), dried over anhydrous sodium sulfate, filtered and the filtrate was concentrated under reduced pressure. The residue was purified by column chromatography to afford the title compound. LCMS R_{T}=1.726 min, m/z=397.2 [M+H]⁺.

### Step 3: 1-tert-butyl 2-methyl 4-(7-chloro-2H-benzo[b][1,4]oxazin-4(3H)-yl)-2-methylpyrrolidine-1,2-dicarboxylate

To a solution of 1-tert-butyl 2-methyl 4-(7-chloro-2H-benzo[b][1,4]oxazin-4(3H)-yl)pyrrolidine-1,2-dicarboxylate (50 g, 125.99 mmol) in THF (400 mL) was added (diisopropylamino)lithium (2 M in THF, 151 mL) at −65° C. After stirred at −65° C. for 1.5 h. Then iodomethane (39 g, 277.17 mmol) was added. After stirred at 20° C. for 12 h, the reaction mixture was quenched by addition of ammonium chloride (200 mL) and extracted with ethyl acetate (400 mL). The combined organic layers were washed with brine (400 mL), dried over anhydrous sodium sulfate, filtered and the filtrate was concentrated under reduced pressure. The residue was purified by column chromatography to afford the title compound. LCMS R_{T}=1.075 min, m/z=411.1 [M+H]⁺.

### Step 4: 1-tert-butyl 2-methyl 4-(5-bromo-7-chloro-2H-benzo[b][1,4]oxazin-4(3H)-yl)-2-methylpyrrolidine-1,2-dicarboxylate

To a solution of 1-tert-butyl 2-methyl 4-(7-chloro-2H-benzo[b][1,4]oxazin-4(3H)-yl)-2-methylpyrrolidine-1,2-dicarboxylate (38 g, 92.48 mmol) in dichloromethane (300 mL) was added N-bromosuccinimide (33 g, 184.96 mmol). After stirred at 20° C. for 1 h, the reaction mixture was quenched by addition of water (100 mL) and extracted with dichloromethane (300 mL). The combined organic layers were washed with brine (300 mL), dried over anhydrous sodium sulfate, filtered and the filtrate was concentrated under reduced pressure. The residue was purified by column chromatography to afford the title compound. LCMS R_{T}=1.988 min, m/z=491.1 [M+2+H]⁺.

### Step 5: tert-butyl 4-(5-bromo-7-chlorochroman-4-yl)-2-(hydroxymethyl)-2-methylpyrrolidine-1-carboxylate

To a solution of 1-tert-butyl 2-methyl 4-(5-bromo-7-chloro-2H-benzo[b][1,4]oxazin-4(3H)-yl)-2-methylpyrrolidine-1,2-dicarboxylate (5 g, 10.21 mmol) in THF (350 mL) was added lithium borohydride (5.3 g, 241.74 mmol). After stirred at 20° C. for 16 h, the reaction mixture was quenched by addition of ammonium chloride (50 mL) and extracted with ethyl acetate (80 mL). The combined organic layers were washed with brine (80 mL), dried over anhydrous sodium sulfate, filtered and the filtrate was concentrated under reduced pressure. The residue was purified by column chromatography to afford the title compound. LCMS R_{T}=1.922 min, m/z=463.1 [M+H]⁺.

### Step 6: tert-butyl 4-(5-bromo-7-chloro-2H-benzo[b][1,4]oxazin-4(3H)-yl)-2-methyl-2-(((tetrahydro-2H-pyran-2-yl)oxy)methyl)pyrrolidine-1-carboxylate

To a solution of tert-butyl 4-(5-bromo-7-chlorochroman-4-yl)-2-(hydroxymethyl)-2-methylpyrrolidine-1-carboxylate (2.9 g, 6.28 mmol) in acetone (30 mL) was added 3,4-dihydro-2H-pyran (2.2 g, 25.10 mmol), 4-methylbenzenesulfonic acid:pyridine (640 mg, 2.51 mmol). After stirred at 50° C. for 3 h, the reaction mixture was filtered and the filtrate was concentrated under reduced pressure. The residue was purified by column chromatography to afford the title compound. LCMS R_{T}=1.050 min, m/z=547.1 [M+H]⁺.

### Step 7: tert-butyl 4-(5-(2-(((tert-butyldimethylsilyl)oxy)methyl)thieno[3,2-b]pyridin-7-yl)-7-chloro-2H-benzo[b][1,4]oxazin-4(3H)-yl)-2-methyl-2-(((tetrahydro-2H-pyran-2-yl)oxy)methyl)pyrrolidine-1-carboxylate

To a solution of tert-butyl 4-(5-bromo-7-chloro-2H-benzo[b][1,4]oxazin-4(3H)-yl)-2-methyl-2-(((tetrahydro-2H-pyran-2-yl)oxy)methyl)pyrrolidine-1-carboxylate (3.1 g, 5.7 mmol) in dioxane (30 mL) and water (3 mL) was added [2-[[tert-butyl(dimethyl)silyl]oxymethyl]thieno[3,2-b]pyridin-7-yl]boronic acid (2.8 g, 8.52 mmol), cyclopenta-2,4-dien-1-yl(diphenyl)phosphane; dichloropalladium; iron(II) (830 mg, 0.01 mmol) and potassium carbonate (2 g, 0.01 mmol). After stirred at 100° C. for 3 h, the residue was purified by column chromatography to afford the title compound. LCMS R_{T}=1.568 min, m/z=465.2 [M+H]⁺.

### Step 8: tert-butyl 4-(5-(2-(((tert-butyldimethylsilyl)oxy)methyl)thieno[3,2-b]pyridin-7-yl)-7-cyano-2H-benzo[b][1,4]oxazin-4(3H)-yl)-2-methyl-2-(((tetrahydro-2H-pyran-2-yl)oxy)methyl)pyrrolidine-1-carboxylate

To a solution of tert-butyl 4-(5-(2-(((tert-butyldimethylsilyl)oxy)methyl)thieno[3,2-b]pyridin-7-yl)-7-chloro-2H-benzo[b][1,4]oxazin-4(3H)-yl)-2-methyl-2-(((tetrahydro-2H-pyran-2-yl)oxy)methyl)pyrrolidine-1-carboxylate (4 g, 5 mmol) in 1-methylpyrrolidin-2-one (40 mL) was added zinc cyanide (2.5 g, 0.02 mmol) and palladium; tritert-butylphosphane (550 mg, 1.07 mmol). After stirred at 165° C. for 1.5 h, the reaction mixture was quenched by addition of water (50 mL) and extracted with ethyl acetate (80 mL). The combined organic layers were washed with brine (80 mL), dried over anhydrous sodium sulfate, filtered and the filtrate was concentrated under reduced pressure. The residue was purified by column chromatography to afford the title compound. LCMS R_{T}=0.990 min, m/z=735.5 [M+H]⁺.

### Step 9: (2R,4S)-tert-butyl 4-(7-cyano-5-(2-(hydroxymethyl)thieno[3,2-b]pyridin-7-yl)-2H-benzo[b][1,4]oxazin-4(3H)-yl)-2-methyl-2-(((tetrahydro-2H-pyran-2-yl)oxy)methyl)pyrrolidine-1-carboxylate

To a solution of tert-butyl 4-(5-(2-(((tert-butyldimethylsilyl)oxy)methyl)thieno[3,2-b]pyridin-7-yl)-7-cyano-2H-benzo[b][1,4]oxazin-4(3H)-yl)-2-methyl-2-(((tetrahydro-2H-pyran-2-yl)oxy)methyl)pyrrolidine-1-carboxylate (3.4 g, 4.63 mmol) in THF (30 mL) was added tetrabutylammonium fluoride (1 M in THF, 6 mL). After stirred at 20° C. for 1 h, the reaction mixture was quenched by addition of water (30 mL) and extracted with ethyl acetate (50 mL). The combined organic layers were washed with brine (50 mL), dried over anhydrous sodium sulfate, filtered and the filtrate was concentrated under reduced pressure. The residue was purified by column chromatography. The racemate was separated by SFC to give first eluting fraction (219.1, Rt=2.956; LCMS R_{T}=0.783 min, m/z=621.3 [M+H]+) and second eluting fraction (219.2, Rt=3.075; LCMS R_{T}=0.787 min, m/z=621.2 [M+H]+).

### Step 10

Example 219 was prepared from (2R,4S)-tert-butyl 4-(7-cyano-5-(2-(hydroxymethyl)thieno[3,2-b]pyridin-7-yl)-2H-benzo[b][1,4]oxazin-4(3H)-yl)-2-methyl-2-(((tetrahydro-2H-pyran-2-yl)oxy)methyl)pyrrolidine-1-carboxylate and 6-(dimethylamino)-1-ethyl-pyrimidine-2,4-dione, following the procedure described in the synthesis of Example 108. The product was purified by preparative HPLC. ¹H NMR (400 MHz, CD30D) 6=8.71 (d, J=4.8 Hz, 1H), 8.47 (s, 1H), 7.53 (s, 1H), 7.44 (d, J=4.8 Hz, 1H), 7.26 (dd, J=11.6 Hz, 2H), 5.34 (br d, J=2.0 Hz, 2H), 5.25 (s, 1H), 4.44-3.68 (m, 6H), 3.39 (s, 2H), 3.26-2.86 (m, 3H), 2.81-2.73 (m, 6H), 1.99-1.62 (m, 2H), LCMS R_{T}=1.289 min, m/z=602.5 [M+H]⁺.

### Example 220: 5-(2-((6,6-dimethyl-2,4-dioxo-3-azabicyclo[3.1.0]hexan-3-yl)methyl)thieno[3,2-b]pyridin-7-yl)-4-((3S,5R)-5-(hydroxymethyl)-5-methylpyrrolidin-3-yl)-3,4-dihydro-2H-benzo[b][1,4]oxazine-7-carbonitrile, formic acid salt

Example 220 was prepared from 219.2 and 6,6-dimethyl-3-azabicyclo[3.1.0]hexane-2,4-dione, following the procedure described in the synthesis of Example 108. The product was purified by preparative HPLC. ¹H NMR (400 MHz, CD30D) 6=8.73 (d, J=4.9 Hz, 1H), 8.50 (s, 1H), 7.57-7.42 (m, 2H), 7.26 (dd, J=1.9, 17.6 Hz, 2H), 4.83 (s, 2H), 4.65-3.53 (m, 4H), 3.52-3.34 (m, 2H), 3.20-2.55 (m, 4H), 2.50 (s, 2H), 1.71 (s, 2H), 1.24 (s, 3H), 1.17 (s, 3H), 1.11 (s, 3H). LCMS R_{T}=1.305 min, m/z=558.4 [M+H]⁺.

### Example 221: 5-[2-[(4-tert-butyl-6-oxo-pyridazin-1-yl)methyl]thieno[3,2-b]pyridin-7-yl]-4-[(3S,5R)-5-(hydroxymethyl)-5-methyl-pyrrolidin-3-yl]-2,3-dihydro-1,4-benzoxazine-7-carbonitrile, formic acid salt

Example 221 was prepared from 219.2 and 5-tert-butyl-4H-pyridazin-3-one, following the procedure described in the synthesis of Example 108. The product was purified by preparative HPLC. ¹H NMR (400 MHz, CD₃OD) δ=8.75-8.71 (m, 1H), 8.51-8.47 (m, 1H), 8.17-8.12 (m, 1H), 7.59-7.55 (m, 1H), 7.48-7.43 (m, 1H), 7.29-7.22 (m, 2H), 6.85-6.81 (m, 1H), 5.70-5.51 (m, 2H), 4.66-3.63 (m, 4H), 3.46-3.36 (m, 1H), 3.21-2.55 (m, 4H), 2.07-1.39 (m, 2H), 1.32-1.30 (m, 9H), 1.21 (s, 3H). LCMS R_{T}=1.493 min, m/z=571.5 [M+H]⁺.

### Example 222: 4-[(3S,5R)-5-(hydroxymethyl)-5-methyl-pyrrolidin-3-yl]-5-[2-[[6-oxo-4-[1-(trifluoromethyl)cyclopropyl]pyridazin-1-yl]methyl]thieno[3,2-b]pyridin-7-yl]-2,3-dihydro-1,4-benzoxazine-7-carbonitrile, formic acid salt

Example 222 was prepared from 219.2 and 4-[1-(trifluoromethyl)cyclopropyl]-1H-pyridazin-6-one, following the procedure described in the synthesis of Example 108. The product was purified by preparative HPLC. ¹H NMR (400 MHz, CD₃OD) δ=8.73 (d, J=4.8 Hz, 1H), 8.04 (d, J=1.6 Hz, 1H), 7.58 (s, 1H), 7.46 (d, J=4.8 Hz, 1H), 7.29-7.21 (m, 2H), 7.10-7.02 (m, 1H), 5.62 (d, J=3.6 Hz, 2H), 4.65-3.44 (m, 4H), 3.18-2.55 (m, 4H), 1.87-1.40 (m, 4H), 1.31-1.09 (m, 6H). LCMS R_{T}=1.500 min, m/z=623.4 [M+H]⁺.

### Example 223: 7-(7-cyano-4-((3S,5R)-5-(hydroxymethyl)-5-methylpyrrolidin-3-yl)-3,4-dihydro-2H-benzo[b][1,4]oxazin-5-yl)-2-((6-oxo-4-(1-(trifluoromethyl)cyclopropyl)pyridazin-1(6H)-yl)methyl)thieno[3,2-b]pyridine 4-oxide

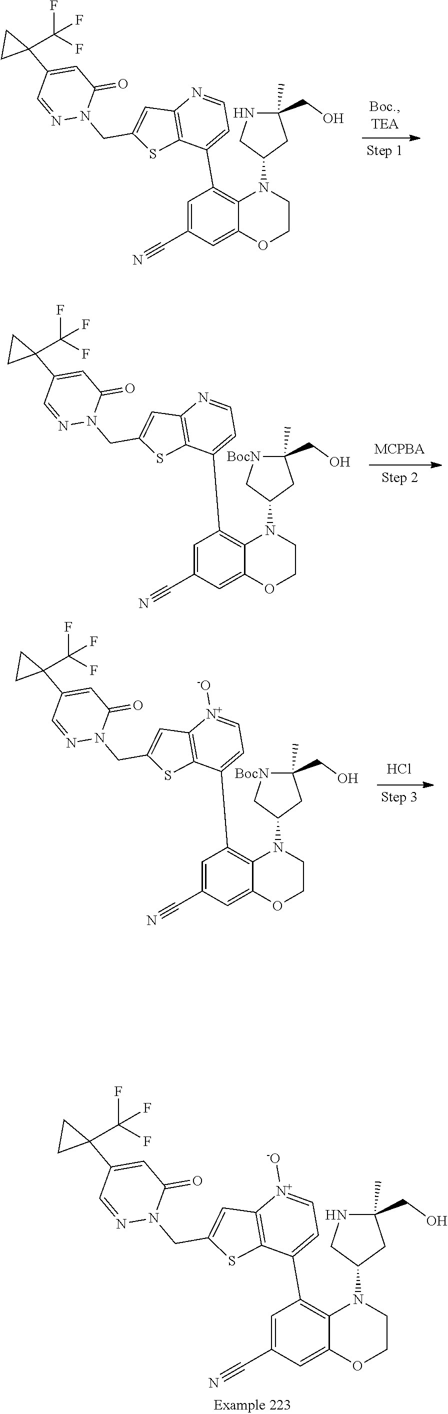

### Step 1: (2R,4S)-tert-butyl 4-(7-cyano-5-(2-((6-oxo-4-(1-(trifluoromethyl)cyclopropyl)pyridazin-1(6H)-yl)methyl)thieno[3,2-b]pyridin-7-yl)-2H-benzo[b][1,4]oxazin-4(3H)-yl)-2-(hydroxymethyl)-2-methylpyrrolidine-1-carboxylate

To a solution of (2R,4S)-tert-butyl 4-(7-cyano-5-(2-((6-oxo-4-(1-(trifluoromethyl)cyclopropyl)pyridazin-1(6H)-yl)methyl)thieno[3,2-b]pyridin-7-yl)-2H-benzo[b][1,4]oxazin-4(3H)-yl)-2-(hydroxymethyl)-2-methylpyrrolidine-1-carboxylate (10 mg, 0.02 mmol) in acetonitrile (2 mL) was added tert-butoxycarbonyl tert-butyl carbonate (4 mg, 0.02 mmol) and triethylamine (2 mg, 0.02 mmol) at 25° C. After stirred at 25° C. for 1 h, the mixture was quenched with water (10 mL), the residue was extracted by ethyl acetate (10 mL×4) and water (1 mL×4), dried over anhydrous sodium sulfate, filtered and the filtrate was concentrated under reduced pressure. The residue was purified by preparative TLC to afford the title compound. LCMS R_{T}=0.539 min, m/z=723.3 [M+H]⁺.

### Step 2: 7-(4-((3S,5R)-1-(tert-butoxycarbonyl)-5-(hydroxymethyl)-5-methylpyrrolidin-3-yl)-7-cyano-3,4-dihydro-2H-benzo[b][1,4]oxazin-5-yl)-2-((6-oxo-4-(1-(trifluoromethyl)cyclopropyl)pyridazin-1(6H)-yl)methyl)thieno[3,2-b]pyridine 4-oxide

To a solution of (2R,4S)-tert-butyl 4-(7-cyano-5-(2-((6-oxo-4-(1-(trifluoromethyl)cyclopropyl)pyridazin-1(6H)-yl)methyl)thieno[3,2-b]pyridin-7-yl)-2H-benzo[b][1,4]oxazin-4(3H)-yl)-2-(hydroxymethyl)-2-methylpyrrolidine-1-carboxylate (4 mg, 0.006 mmol) in dichloromethane (1 mL) was added metachloroperbenzoic acid (2 mg, 0.01 mol) at 0° C. After stirred for 4 h at 0° C., the reaction mixture was concentrated under reduced pressure. The residue was purified by preparative HPLC to afford the title compound. LCMS R_{T}=2.828 min, m/z=739.5 [M+H]⁺.

### Step 3

The solution of 7-(4-((3S,5R)-1-(tert-butoxycarbonyl)-5-(hydroxymethyl)-5-methylpyrrolidin-3-yl)-7-cyano-3,4-dihydro-2H-benzo[b][1,4]oxazin-5-yl)-2-((6-oxo-4-(1-(trifluoromethyl)cyclopropyl)pyridazin-1(6H)-yl)methyl)thieno[3,2-b]pyridine 4-oxide (3 mg, 0.004 mmol) dissolved in dioxane (1 mL) and hydrochloric acid (0.5 mL, 4 M in dioxane) was added. After stirred at 25° C. for 1 h, the reaction mixture was concentrated under reduced pressure. The residue was purified by preparative HPLC to afford Example 223. ¹H NMR (400 MHz, CD₃OD) δ=8.56-8.47 (m, 1H), 8.15-8.04 (m, 1H), 7.91-7.79 (m, 1H), 7.61-7.48 (m, 1H), 7.40-7.25 (m, 2H), 7.14-7.07 (m, 1H), 5.65 (s, 2H), 4.57-3.41 (m, 5H), 3.20-2.37 (m, 4H), 1.67-1.41 (m, 3H), 1.34-0.98 (m, 6H). LCMS R_{T}=1.976 min, m/z=639.4 [M+H]⁺

### Example 224: 1-((7-(4-((3S,5R)-5-(hydroxymethyl)-5-methylpyrrolidin-3-yl)-7-(trifluoromethyl)-3,4-dihydro-2H-benzo[b][1,4]oxazin-5-yl)thieno[3,2-b]pyridin-2-yl)methyl)pyrrolidine-2,5-dione, hydrochloride salt

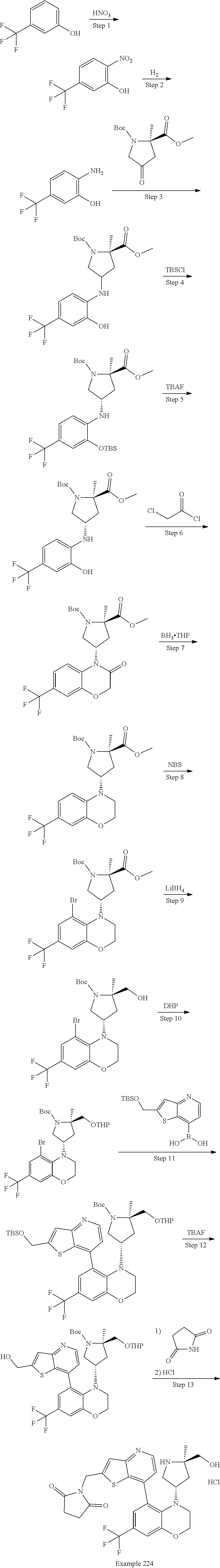

### Step 1: 2-nitro-5-(trifluoromethyl)phenol

To a solution of 3-(trifluoromethyl)phenol (20 g, 123.37 mmol) in acetic acid (150 mL) was added nitric acid (14.35 g, 148.05 mmol) in acetic acid (90 mL). After stirred at 40° C. for 1 h, the mixture was concentrated under reduced pressure. The residue was diluted with water (200 mL) and extracted with ethyl acetate (100 mL×3). The combined organic layers were washed with brine (250 mL), dried over anhydrous sodium sulfate and concentrated under reduced pressure. The residue was purified by column chromatography to afford the title compound. ¹H NMR (400 MHz, CDCl₃) δ=10.65-10.58 (m, 1H), 8.33-8.24 (m, 1H), 7.52-7.45 (m, 1H), 7.31-7.26 (m, 1H).

### Step 2: 2-amino-5-(trifluoromethyl)phenol

To a solution of 2-nitro-5-(trifluoromethyl)phenol (6 g, 28.97 mmol) in ethyl alcohol (60 mL) was added 10% palladium on carbon (1.3 g, 28.97 mmol). After stirred at 80° C. for 5 h under hydrogen atmosphere (15 psi), the mixture was filtered and the filtrate was concentrated under reduced pressure. The residue was purified by column chromatography to afford the title compound. LCMS R_{T}=0.730 min, m/z=178.1 [M+H]⁺.

### Step 3: (2R)-1-tert-butyl 2-methyl 4-((2-hydroxy-4-(trifluoromethyl)phenyl)amino)-2-methylpyrrolidine-1,2-dicarboxylate

To a solution of 2-amino-5-(trifluoromethyl)phenol (5 g, 28.23 mmol) in 1,2-dichloroethane (100 mL) was added sodium triacetoxyborohydride (11.97 g, 56.46 mmol), (R)-1-tert-butyl 2-methyl 2-methyl-4-oxopyrrolidine-1,2-dicarboxylate (7.99 g, 31.05 mmol) and acetic acid (1.70 g, 28.23 mmol). After stirred at 25° C. for 2 h, the mixture was concentrated under reduced pressure. The residue was purified by column chromatography to afford the title compound. LCMS R_{T}=0.857 min, m/z=419.1 [M+H]⁺.

### Step 4: (2R,4S)-1-tert-butyl 2-methyl 4-((2-((tert-butyldimethylsilyl)oxy)-4-(trifluoromethyl)phenyl)amino)-2-methylpyrrolidine-1,2-dicarboxylate

To a solution of (2R)-1-tert-butyl 2-methyl 4-((2-hydroxy-4-(trifluoromethyl)phenyl)amino)-2-methylpyrrolidine-1,2-dicarboxylate (10 g, 23.90 mmol) in dichloromethane (120 mL) was added imidazole (3.25 g, 47.80 mmol) and tert-butyldimethylsilyl chloride (5.40 g, 35.85 mmol). After stirred at 25° C. for 3 h, the mixture was diluted with water (100 mL) and extracted with dichloromethane (80 mL×3). The combined organic layers were washed with brine (150 mL), dried over anhydrous sodium sulfate and concentrated under reduced pressure. The residue was purified by column chromatography to afford the title compound. LCMS R_{T}=0.967 min, m/z=533.2 [M+H]⁺.

### Step 5: (2R,4S)-1-tert-butyl 2-methyl 4-((2-hydroxy-4-(trifluoromethyl)phenyl)amino)-2-methylpyrrolidine-1,2-dicarboxylate

To a solution of (2R,4S)-1-tert-butyl 2-methyl 4-((2-((tert-butyldimethylsilyl)oxy)-4-(trifluoromethyl)phenyl)amino)-2-methylpyrrolidine-1,2-dicarboxylate (2.7 g, 5.07 mmol) in THF (20 mL) was added tetrabutylammonium fluoride (5.1 mL, 1.0 M in THF). After stirred at 25° C. for 12 h, the mixture was diluted with water (20 mL) and extracted with ethyl acetate (150 mL×3). The combined organic layers were washed with brine (25 mL), dried over anhydrous sodium sulfate and concentrated under reduced pressure. The residue was purified by column chromatography to afford the title compound. LCMS R_{T}=0.568 min, m/z=419.1 [M+H]⁺.

### Step 6: (2R,4S)-1-tert-butyl 2-methyl 2-methyl-4-(3-oxo-7-(trifluoromethyl)-2H-benzo[b][1,4]oxazin-4(3H)-yl)pyrrolidine-1,2-dicarboxylate

To a solution of (2R,4S)-1-tert-butyl 2-methyl 4-((2-hydroxy-4-(trifluoromethyl)phenyl)amino)-2-methylpyrrolidine-1,2-dicarboxylate (1.3 g, 3.11 mmol) in acetone (20 mL) was added potassium carbonate (1.29 g, 9.32 mmol) and 2-chloroacetyl chloride (526 mg, 4.66 mmol). After stirred at 50° C. for 2 h, the mixture was concentrated under reduced pressure. The residue was diluted with water (30 mL) and extracted with ethyl acetate (20 mL×3). The combined organic layers were washed with brine (30 mL), dried over anhydrous sodium sulfate and concentrated under reduced pressure. The residue was purified by column chromatography to afford the title compound. LCMS R_{T}=0.607 min, m/z=359.0 [M+H−Boc]⁺.

### Step 7: (2R,4S)-1-tert-butyl 2-methyl 2-methyl-4-(7-(trifluoromethyl)-2H-benzo[b][1,4]oxazin-4(3H)-yl)pyrrolidine-1,2-dicarboxylate

To a solution of (2R,4S)-1-tert-butyl 2-methyl 2-methyl-4-(3-oxo-7-(trifluoromethyl)-2H-benzo[b][1,4]oxazin-4(3H)-yl)pyrrolidine-1,2-dicarboxylate (1.4 g, 3.05 mmol) in THF (20 mL) was added borane (9.16 mL, 1 M in THF). After stirred at 25° C. for 2 h, the mixture was quenched by addition of methanol (20 mL) and concentrated under reduced pressure. The residue was purified by column chromatography to afford the title compound. LCMS R_{T}=1.080 min, m/z=445.2 [M+H]⁺.

### Step 8: (2R,4S)-1-tert-butyl 2-methyl 4-(5-bromo-7-(trifluoromethyl)-2H-benzo[b][1,4]oxazin-4(3H)-yl)-2-methylpyrrolidine-1,2-dicarboxylate

To a solution of (2R,4S)-1-tert-butyl 2-methyl 2-methyl-4-(7-(trifluoromethyl)-2H-benzo[b][1,4]oxazin-4(3H)-yl)pyrrolidine-1,2-dicarboxylate (1.34 g, 3.02 mmol) in dichloromethane (20 mL) was added N-bromosuccinimide (590 mg, 3.32 mmol) at 0° C. After stirred at 0° C. for 1 h, the mixture was diluted with water (20 mL) and extracted with dichloromethane (15 mL×2). The combined organic layers were washed with brine (25 mL), dried over anhydrous sodium sulfate and concentrated under reduced pressure. The residue was purified by column chromatography to afford the title compound. LCMS R_{T}=0.643 min, m/z=424.8 [M+H−Boc]⁺.

### Step 9: (2R,4S)-tert-butyl 4-(5-bromo-7-(trifluoromethyl)-2H-benzo[b][1,4]oxazin-4(3H)-yl)-2-(hydroxymethyl)-2-methylpyrrolidine-1-carboxylate

To a solution of (2R,4S)-1-tert-butyl 2-methyl 4-(5-bromo-7-(trifluoromethyl)-2H-benzo[b][1,4]oxazin-4(3H)-yl)-2-methylpyrrolidine-1,2-dicarboxylate (1.3 g, 2.48 mmol) in THF (20 mL) was added lithium borohydride (284 mg, 13.05 mmol). After stirred at −20° C. for 3 h, the mixture was diluted with saturated ammonium chloride (20 mL) and extracted with ethyl acetate (15 mL×3). The combined organic layers were washed with brine (25 mL), dried over anhydrous sodium sulfate and concentrated under reduced pressure. The residue was purified by column chromatography to afford the title compound. LCMS R_{T}=1.063 min, m/z=497.0 [M+H]⁺.

### Step 10: (2R,4S)-tert-butyl 4-(5-bromo-7-(trifluoromethyl)-2H-benzo[b][1,4]oxazin-4(3H)-yl)-2-methyl-2-(((tetrahydro-2H-pyran-2-yl)oxy)methyl)pyrrolidine-1-carboxylate

To a solution of (2R,4S)-tert-butyl 4-(5-bromo-7-(trifluoromethyl)-2H-benzo[b][1,4]oxazin-4(3H)-yl)-2-(hydroxymethyl)-2-methylpyrrolidine-1-carboxylate (1 g, 2.02 mmol) in acetone (20 mL) was added (3,4-dihydro-2H-pyran-2-yl)methanol (509 mg, 6.06 mmol) and pyridinium p-toluenesulfonate (152 mg, 0.61 mmol). After stirred at 50° C. for 3 h, the mixture was concentrated under reduced pressure. The residue was purified by column chromatography to afford the title compound. LCMS R_{T}=0.675 min, m/z=579.2 [M+H]⁺.

### Step 11: (2R,4S)-tert-butyl 4-(5-(2-(((tert-butyldimethylsilyl)oxy)methyl)thieno[3,2-b]pyridin-7-yl)-7-(trifluoromethyl)-2H-benzo[b][1,4]oxazin-4(3H)-yl)-2-methyl-2-(((tetrahydro-2H-pyran-2-yl)oxy)methyl)pyrrolidine-1-carboxylate

To a solution of (2R,4S)-tert-butyl 4-(5-bromo-7-(trifluoromethyl)-2H-benzo[b][1,4]oxazin-4(3H)-yl)-2-methyl-2-(((tetrahydro-2H-pyran-2-yl)oxy)methyl)pyrrolidine-1-carboxylate (1.15 g, 1.99 mmol) in dioxane (30 mL) and water (3 mL) was added potassium carbonate (550 mg, 3.98 mmol), (2-(((tert-butyldimethylsilyl)oxy)methyl)thieno[3,2-b]pyridin-7-yl)boronic acid (966 mg, 2.99 mmol) and dichloro[1,1′-bis(diphenylphosphino)ferrocene]palladium(II) (291 mg, 0.40 mmol). After stirred at 95° C. for 3 h, the mixture was filtered and the filtrate was concentrated under reduced pressure. The residue was purified by column chromatography to afford the title compound. LCMS R_{T}=2.363 min, m/z=778.4 [M+H]⁺.

### Step 12: (2R,4S)-tert-butyl 4-(5-(2-(hydroxymethyl)thieno[3,2-b]pyridin-7-yl)-7-(trifluoromethyl)-2H-benzo[b][1,4]oxazin-4(3H)-yl)-2-methyl-2-(((tetrahydro-2H-pyran-2-yl)oxy)methyl)pyrrolidine-1-carboxylate

To a solution of (2R,4S)-tert-butyl 4-(5-(2-(((tert-butyldimethylsilyl)oxy)methyl)thieno[3,2-b]pyridin-7-yl)-7-(trifluoromethyl)-2H-benzo[b][1,4]oxazin-4(3H)-yl)-2-methyl-2-(((tetrahydro-2H-pyran-2-yl)oxy)methyl)pyrrolidine-1-carboxylate (300 mg, 0.39 mmol) in THF (8 mL) was added tetrabutylammonium fluoride (0.4 mL, 1.0 M in THF). After stirred at 25° C. for 12 h, the mixture was diluted with water (20 mL) and extracted with ethyl acetate (15 mL×3). The combined organic layers were washed with brine (25 mL), dried over anhydrous sodium sulfate and concentrated under reduced pressure. The residue was purified by column chromatography to afford the title compound. LCMS R_{T}=0.564 min, m/z=664.4 [M+H]⁺.

### Step 13

Example 224 was prepared from (2R,4S)-tert-butyl 4-(5-(2-(hydroxymethyl)thieno[3,2-b]pyridin-7-yl)-7-(trifluoromethyl)-2H-benzo[b][1,4]oxazin-4(3H)-yl)-2-methyl-2-(((tetrahydro-2H-pyran-2-yl)oxy)methyl)pyrrolidine-1-carboxylate and succinimide, following the procedure described in the synthesis of Example 108. The product was purified by preparative HPLC. ¹H NMR (400 MHz, CD₃OD) δ=8.98-8.92 (m, 1H), 8.03-8.01 (m, 1H), 7.77 (s, 1H), 7.33 (d, J=9.2 Hz, 2H), 5.10-5.04 (m, 2H), 4.44-4.10 (m, 2H), 3.93-3.67 (m, 1H), 3.47 (d, J=8.0 Hz, 2H), 3.29-2.76 (m, 8H), 2.25-1.51 (m, 2H), 1.29 (s, 3H). LCMS R_{T}=1.533 min, m/z=561.1 [M+H]⁺.

### Example 225: 5-(2-((4-(tert-butyl)-6-oxopyridazin-1(6H)-yl)methyl)thieno[3,2-b]pyridin-7-yl)-4-((3S,5R)-5-(hydroxymethyl)-5-methylpyrrolidin-3-yl)-3-oxo-3,4-dihydro-2H-benzo[b][1,4]oxazine-7-carbonitrile, formic acid salt

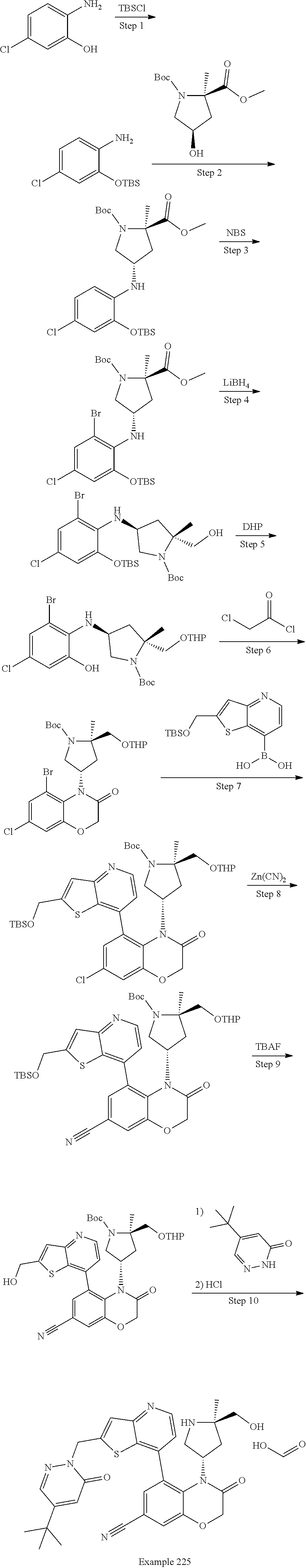

### Step 1: 2-((tert-butyldimethylsilyl)oxy)-4-chloroaniline

To a solution of 2-amino-5-chlorophenol (5 g, 34.83 mmol) in dichloromethane (90 mL) was added imidazole (4.74 g, 69.65 mmol) and tert-butylchlorodimethylsilane (7.87 g, 52.24 mmol) at 0° C. After stirred at 0° C. for 1 h, the mixture was washed with hydrochloric acid (0.5 M, 50 mL), saturated aqueous sodium bicarbonate (50 mL) and brine (50 mL). The organic layer was dried over anhydrous sodium sulfate, filtered and the filtrate was concentrated under reduced pressure. The residue was purified by column chromatography to afford the title compound. ¹H NMR (400 MHz, CD₃OD) δ=6.76-6.66 (m, 3H), 1.05-0.99 (m, 9H), 0.28-0.22 (m, 6H).

### Step 2: (2R,4S)-1-tert-butyl 2-methyl 4-((2-((tert-butyldimethylsilyl)oxy)-4-chlorophenyl)amino)-2-methylpyrrolidine-1,2-dicarboxylate

To a solution of 2-((tert-butyldimethylsilyl)oxy)-4-chloroaniline (30 mL) was added N,N-diisopropylethylamine (2.24 g, 17.35 mmol) and trifluoromethanesulfonic anhydride (3.59 g, 12.73 mmol) at 0° C. After stirred at 0° C. for 0.5 h, then 2-((tert-butyldimethylsilyl)oxy)-4-chloroaniline (4.47 g, 17.35 mmol) was added at 0° C. After stirred at 40° C. for 2.5 h, the mixture was quenched by addition of saturated aqueous sodium bicarbonate (50 mL) and extracted with dichloromethane (40 mL). The organic layer was washed with brine (50 mL), dried over anhydrous sodium sulfate, filtered and the filtrate was concentrated under reduced pressure. The residue was purified by column chromatography to afford the title compound. LCMS R_{T}=2.137 min, m/z=499.3 [M+H]⁺.

### Step 3: (2R,4S)-1-tert-butyl 2-methyl 4-((2-bromo-6-((tert-butyldimethylsilyl)oxy)-4-chlorophenyl)amino)-2-methylpyrrolidine-1,2-dicarboxylate

To a solution of (2R,4S)-1-tert-butyl 2-methyl 4-((2-((tert-butyldimethylsilyl)oxy)-4-chlorophenyl)amino)-2-methylpyrrolidine-1,2-dicarboxylate (2.3 g, 4.61 mmol) in acetonitrile (30 mL) was added 1-bromopyrrolidine-2,5-dione (861 mg, 4.84 mmol) at 20° C. After stirred at 20° C. for 1 h, the mixture was quenched by addition of saturated aqueous sodium bicarbonate (20 mL) and water (30 mL). The mixture was extracted with ethyl acetate (30 mL×2). The combined organic layers were washed with brine (50 mL), dried over anhydrous sodium sulfate, filtered and the filtrate was concentrated under reduced pressure. The residue was purified by column chromatography to afford the title compound. LCMS R_{T}=2.317 min, m/z=579.2 [M+2+H]⁺.

### Step 4: (2R,4S)-tert-butyl 4-((2-bromo-6-((tert-butyldimethylsilyl)oxy)-4-chlorophenyl)amino)-2-(hydroxymethyl)-2-methylpyrrolidine-1-carboxylate

To a solution of (2R,4S)-1-tert-butyl 2-methyl 4-((2-bromo-6-((tert-butyldimethylsilyl)oxy)-4-chlorophenyl)amino)-2-methylpyrrolidine-1,2-dicarboxylate (1.9 g, 3.29 mmol) in THF (40 mL) was added lithium borohydride (330 mg, 15.15 mmol) at 0° C. After stirred at 15° C. for 4 h, the mixture was quenched by addition of hydrochloric acid (0.5 M, 50 mL) and extracted with ethyl acetate (40 mL×2). The combined organic layers were washed with brine (50 mL), dried over anhydrous sodium sulfate, filtered and the filtrate was concentrated under reduced pressure. The residue was purified by column chromatography to afford the title compound. ¹H NMR (400 MHz, CDCl₃) δ=7.14 (d, J=1.6 Hz, 1H), 6.74 (d, J=2.0 Hz, 1H), 4.59-4.25 (m, 2H), 3.78-3.56 (m, 3H), 3.27 (dd, J=5.2, 11.2 Hz, 1H), 2.10 (dd, J=6.4, 12.4 Hz, 1H), 1.89-1.68 (m, 2H), 1.46 (s, 9H), 1.30-1.25 (m, 2H), 1.05-0.99 (m, 10H), 0.26 (d, J=2.0 Hz, 6H).

### Step 5: (2R,4S)-tert-butyl 4-((2-bromo-4-chloro-6-hydroxyphenyl)amino)-2-methyl-2-(((tetrahydro-2H-pyran-2-yl)oxy)methyl)pyrrolidine-1-carboxylate

To a solution of (2R,4S)-tert-butyl 4-((2-bromo-6-((tert-butyldimethylsilyl)oxy)-4-chlorophenyl)amino)-2-(hydroxymethyl)-2-methylpyrrolidine-1-carboxylate (1.2 g, 2.18 mmol) in acetone (20 mL) were added (3,4-dihydro-2H-pyran-2-yl)methanol (551 mg, 6.55 mmol) and pyridinium p-toluenesulfonate (219 mg, 0.87 mmol). After stirred at 45° C. for 8 h, the mixture was quenched by addition of saturated sodium bicarbonate (40 mL) and extracted with ethyl acetate (30 mL×2). The combined organic layers were washed with brine (50 mL), dried over anhydrous sodium sulfate and concentrated under reduced pressure. The residue was purified by column chromatography to afford the title compound. LCMS R_{T}=1.653 min, m/z=521.3 [M+2+H]⁺.

### Step 6: (2R,4S)-tert-butyl 4-(5-bromo-7-chloro-3-oxo-2H-benzo[b][1,4]oxazin-4(3H)-yl)-2-methyl-2-(((tetrahydro-2H-pyran-2-yl)oxy)methyl)pyrrolidine-1-carboxylate

To a solution of (2R,4S)-tert-butyl 4-((2-bromo-4-chloro-6-hydroxyphenyl)amino)-2-methyl-2-(((tetrahydro-2H-pyran-2-yl)oxy)methyl)pyrrolidine-1-carboxylate (650 mg, 1.25 mmol) in acetonitrile (15 mL) were added potassium carbonate (518 mg, 3.75 mmol) and 2-chloroacetyl chloride (212 mg, 1.88 mmol). After stirred at 50° C. for 1 h, the mixture was concentrated under reduced pressure. The residue was diluted with water (50 mL) and extracted with ethyl acetate (40 mL×3). The combined organic layers were washed with brine (50 mL), dried over anhydrous sodium sulfate and concentrated under reduced pressure. The residue was purified by column chromatography to afford the title compound. LCMS R_{T}=2.283 min, m/z=583.2 [M+2+Na]⁺.

### Step 7: (2R,4S)-tert-butyl 4-(5-(2-(((tert-butyldimethylsilyl)oxy)methyl)thieno[3,2-b]pyridin-7-yl)-7-chloro-3-oxo-2H-benzo[b][1,4]oxazin-4(3H)-yl)-2-methyl-2-(((tetrahydro-2H-pyran-2-yl)oxy)methyl)pyrrolidine-1-carboxylate

To a solution of (2R,4S)-tert-butyl 4-(5-bromo-7-chloro-3-oxo-2H-benzo[b][1,4]oxazin-4(3H)-yl)-2-methyl-2-(((tetrahydro-2H-pyran-2-yl)oxy)methyl)pyrrolidine-1-carboxylate (480 mg, 0.86 mmol) in dioxane (15 mL) and water (1 mL) were added potassium carbonate (296 mg, 2.14 mmol), [2-[[tert-butyl(dimethyl)silyl]oxymethyl]thieno[3,2-b]pyridin-7-yl]boronic acid (305 mg, 0.94 mmol) and dichloro[1,1′-bis(diphenylphosphino)ferrocene]palladium(II) (125 mg, 0.17 mmol). After stirred at 95° C. for 1.5 h, the mixture was filtered and the filtrate was concentrated under reduced pressure. The residue was purified by column chromatography to afford the title compound. LCMS R_{T}=1.985 min, m/z=758.3 [M+H]⁺.

### Step 8: (2R,4S)-tert-butyl 4-(5-(2-(((tert-butyldimethylsilyl)oxy)methyl)thieno[3,2-b]pyridin-7-yl)-7-cyano-3-oxo-2H-benzo[b][1,4]oxazin-4(3H)-yl)-2-methyl-2-(((tetrahydro-2H-pyran-2-yl)oxy)methyl)pyrrolidine-1-carboxylate

To a solution of (2R,4S)-tert-butyl 4-(5-(2-(((tert-butyldimethylsilyl)oxy)methyl)thieno[3,2-b]pyridin-7-yl)-7-chloro-3-oxo-2H-benzo[b][1,4]oxazin-4(3H)-yl)-2-methyl-2-(((tetrahydro-2H-pyran-2-yl)oxy)methyl)pyrrolidine-1-carboxylate (400 mg, 0.53 mmol) in 1-methylpyrrolidin-2-one (10 mL) were added zinc cyanide (1.1 g, 9.37 mmol) and bis(tri-tert-butylphosphine) palladium(0) (54 mg, 0.11 mmol). After stirred at 160° C. for 1.5 h under microwave, the mixture was diluted with ethyl acetate (70 mL), washed with water (50 mL) and brine (60 mL×2). The organic layer was dried over anhydrous sodium sulfate, filtered and the filtrate was concentrated under reduced pressure. The residue was purified by column chromatography to afford the title compound. LCMS R_{T}=2.323 min, m/z=749.3 [M+H]⁺.

### Step 9: (2R,4S)-tert-butyl 4-(7-cyano-5-(2-(hydroxymethyl)thieno[3,2-b]pyridin-7-yl)-3-oxo-2H-benzo[b][1,4]oxazin-4(3H)-yl)-2-methyl-2-(((tetrahydro-2H-pyran-2-yl)oxy)methyl)pyrrolidine-1-carboxylate

To a solution of (2R,4S)-tert-butyl 4-(5-(2-(((tert-butyldimethylsilyl)oxy)methyl)thieno[3,2-b]pyridin-7-yl)-7-cyano-3-oxo-2H-benzo[b][1,4]oxazin-4(3H)-yl)-2-methyl-2-(((tetrahydro-2H-pyran-2-yl)oxy)methyl)pyrrolidine-1-carboxylate (240 mg, 0.32 mmol) in THF (2 mL) was added tetrabutylammonium fluoride (0.3 mL, 1.0 M in THF). After stirred at 20° C. for 1 h, the mixture was concentrated under reduced pressure. The residue was purified by column chromatography to afford the title compound. LCMS R_{T}=1.743 min, m/z=635.3 [M+H]⁺.

### Step 10

Example 225 was prepared from (2R,4S)-tert-butyl 4-(7-cyano-5-(2-(hydroxymethyl)thieno[3,2-b]pyridin-7-yl)-3-oxo-2H-benzo[b][1,4]oxazin-4(3H)-yl)-2-methyl-2-(((tetrahydro-2H-pyran-2-yl)oxy)methyl)pyrrolidine-1-carboxylate (40 mg, 0.06 mmol) and 5-(tert-butyl)pyridazin-3(2H)-one, following the procedure described in the synthesis of Example 108. The product was purified by preparative HPLC. ¹H NMR (400 MHz, CD3DO) 6=8.81 (t, J=5.2 Hz, 1H), 8.52 (s, 1H), 8.12 (s, 1H), 7.69-7.50 (m, 4H), 6.82 (d, J=2.4 Hz, 1H), 5.69-5.53 (m, 2H), 4.79-4.74 (m, 1H), 4.70-4.62 (m, 1H), 4.10-3.93 (m, 1H), 3.27-3.05 (m, 2H), 2.97-2.89 (m, 1H), 2.55-1.96 (m, 1H), 1.92-1.79 (m, 1H), 1.59-0.62 (m, 13H). LCMS R_{T}=0.462 min, m/z=585.3 [M+H]⁺.

### Example 226: 4-[(3S,5R)-5-(hydroxymethyl)-5-methyl-pyrrolidin-3-yl]-3-oxo-5-[2-[[6-oxo-4-[1-(trifluoromethyl)cyclopropyl]pyridazin-1-yl]methyl]thieno[3,2-b]pyridin-7-yl]-1,4-benzoxazine-7-carbonitrile, formic acid salt

Example 226 was prepared from tert-butyl (2R,4S)-4-[7-cyano-5-[2-(hydroxymethyl)thieno[3,2-b]pyridin-7-yl]-3-oxo-1,4-benzoxazin-4-yl]-2-methyl-2-(tetrahydropyran-2-yloxymethyl)pyrrolidine-1-carboxylate and 4-[1-(trifluoromethyl)cyclopropyl]-1H-pyridazin-6-one, following the procedure described in the synthesis of Example 108. The product was purified by preparative HPLC. ¹H NMR (400 MHz, DMSO-d₆) δ=8.81 (dd, J=4.4, 14.0 Hz, 1H), 8.20 (s, 1H), 7.98 (s, 1H), 7.83 (d, J=5.6 Hz, 1H), 7.75-7.52 (m, 3H), 7.05 (s, 1H), 5.54 (s, 2H), 4.86-4.72 (m, 1H), 4.55 (t, J=13.6 Hz, 1H), 2.71-2.60 (m, 2H), 2.36-2.29 (m, 1H), 2.25-2.06 (m, 1H), 1.53 (d, J=8.4 Hz, 1H), 1.43-1.11 (m, 5H), 0.98-0.84 (m, 3H), 0.48-0.36 (m, 1H). LCMS R_{T}=0.469 min, m/z=637.2 [M+H]⁺.

### Example 227a, 227b, 227c and 227d: 1-((7-(7-chloro-4-((3R,5S)-5-(hydroxymethyl)THF-3-yl)-3,4-dihydro-2H-benzo[b][1,4]oxazin-5-yl)thieno[3,2-b]pyridin-2-yl)methyl)pyrrolidine-2,5-dione 1-((7-(7-chloro-4-((3R,5R)-5-(hydroxymethyl)THF-3-yl)-3,4-dihydro-2H-benzo[b][1,4]oxazin-5-yl)thieno[3,2-b]pyridin-2-yl)methyl)pyrrolidine-2,5-dione 1-[[7-[7-chloro-4-[(3S,5S)-5-(hydroxymethyl)THF-3-yl]-2,3-dihydro-1,4-benzoxazin-5-yl]thieno[3,2-b]pyridin-2-yl]methyl]pyrrolidine-2,5-dione 1-((7-(7-chloro-4-((3S,5R)-5-(hydroxymethyl)THF-3-yl)-3,4-dihydro-2H-benzo[b][1,4]oxazin-5-yl)thieno[3,2-b]pyridin-2-yl)methyl)pyrrolidine-2,5-dione

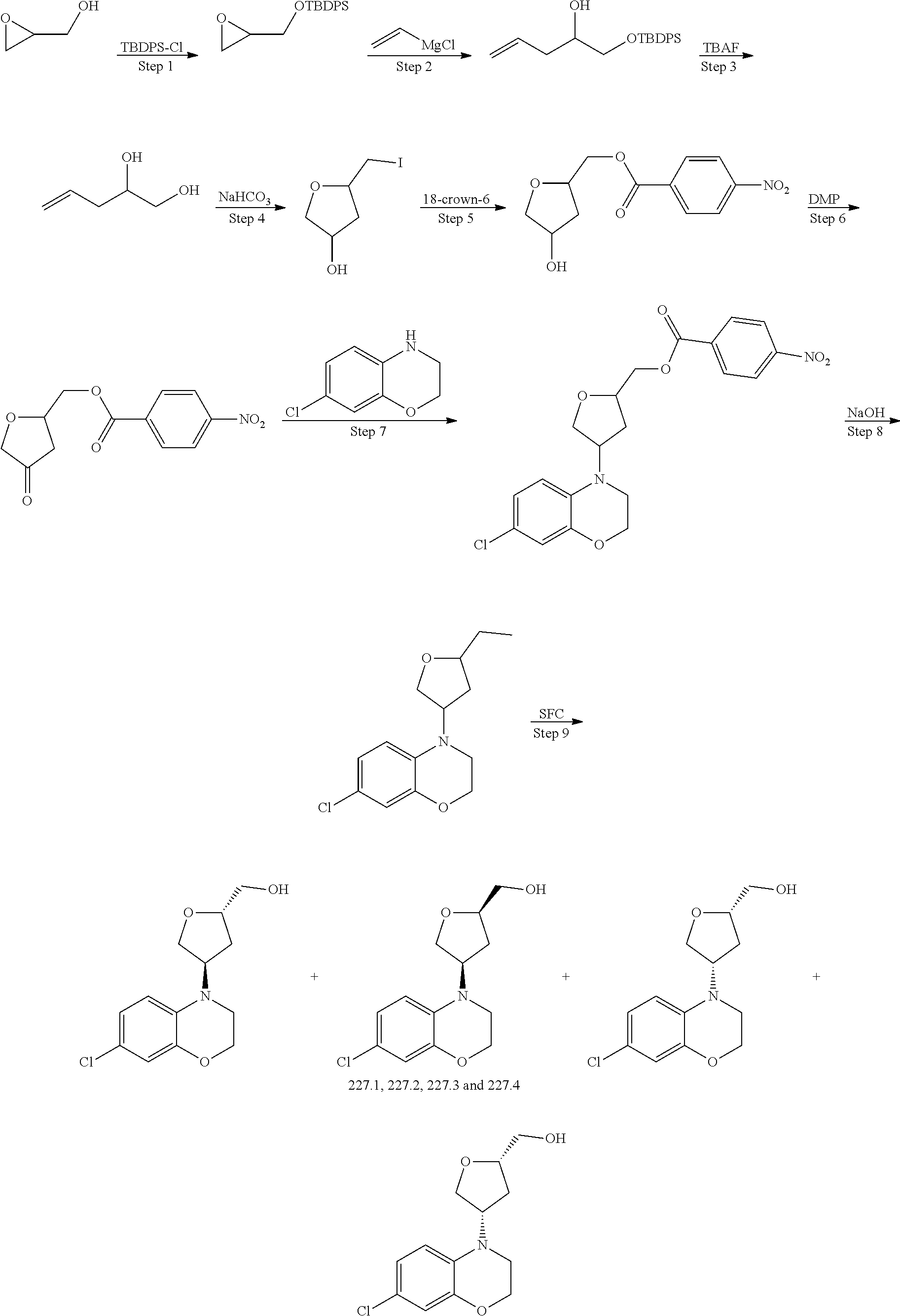

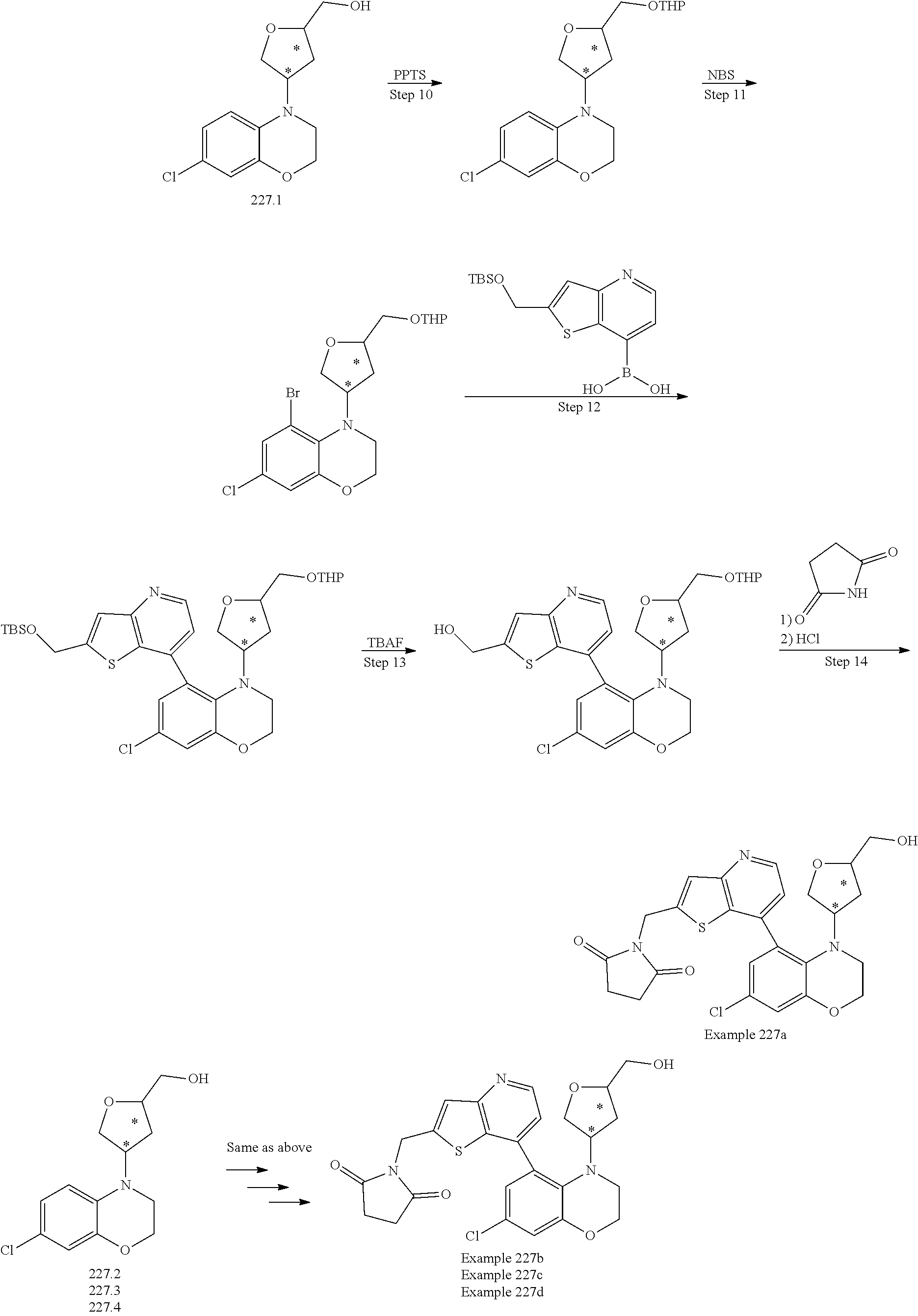

### Step 1: tert-butyl(oxiran-2-ylmethoxy)diphenylsilane

To a solution of [(2R)-oxiran-2-yl]methanol (12 g, 161.99 mmol) in dichloromethane (300 mL) was added tert-butyl-chloro-diphenyl-silane (57.9 g, 210.59 mmol) and imidazole (27.6 g, 404.98 mmol) at 20° C. After stirred at 20° C. for 6 h, the reaction mixture was diluted with water (300 mL) and extracted with ethyl acetate (100 mL×4). The combined organic layers were washed with brine (100 mL), dried over anhydrous sodium sulfate, filtered and the filtrate was concentrated under reduced pressure. The residue was purified by column chromatography to afford the title compound. ¹H NMR (400 MHz, CD₃OD) δ=7.73-7.67 (m, 4H), 7.47-7.37 (m, 6H), 3.92-3.81 (m, 1H), 3.79-3.66 (m, 1H), 3.19-3.09 (m, 1H), 2.81-2.70 (m, 1H), 2.67-2.58 (m, 1H), 1.07 (s, 9H).

### Step 2: 1-[tert-butyl(diphenyl)silyl]oxypent-4-en-2-ol

To a solution of copper(I) cyanide (19.4 g, 216.02 mmol) in THF (200 mL) was added bromo(vinyl)magnesium (432 mL, 1 M in THF) at −78° C., the mixture was stirred at −20° C. for 0.5 h, then was added tert-butyl-[[(2S)-oxiran-2-yl]methoxy]-diphenyl-silane (45 g, 144.01 mmol) in THF (200 mL) at −78° C. After stirred at 20° C. for 7.5 h, the reaction mixture was quenched by addition of saturated aqueous ammonium chloride (400 mL) and extracted with ethyl acetate (400 mL×3). The combined organic layers were washed with brine (200 mL), dried over anhydrous sodium sulfate, filtered and the filtrate was concentrated under reduced pressure. The residue was purified by column chromatography to afford the title compound. ¹H NMR (400 MHz, CD₃OD) δ=7.71-7.64 (m, 4H), 7.46-7.36 (m, 6H), 5.87-5.74 (m, 1H), 5.13-5.03 (m, 2H), 3.85-3.75 (m, 1H), 3.72-3.65 (m, 1H), 3.60-3.52 (m, 1H), 2.50-2.42 (m, 1H), 2.30-2.21 (m, 2H), 1.10-1.07 (m, 9H).

### Step 3: pent-4-ene-1,2-diol

To a solution of (2S)-1-[tert-butyl(diphenyl)silyl]oxypent-4-en-2-ol (40 g, 117.46 mmol) in THF (60 mL) was added tetrabutylammonium fluoride (129 mL, 1 M in THF) at 20° C. After stirred at 20° C. for 1 h, the reaction mixture was concentrated under reduced pressure to remove solvent. The residue was purified by column chromatography to afford the title compound. ¹H NMR (400 MHz, CD₃OD) δ=5.92-5.73 (m, 1H), 5.23-5.04 (m, 2H), 3.81-3.73 (m, 1H), 3.66 (dd, J=11.2 Hz, 3.2 Hz, 11.2 Hz, 1H), 3.47 (dd, J=11.2 Hz, 7.6 Hz, 1H), 2.74 (s, 2H), 2.32-2.16 (m, 2H).

### Step 4: 5-(iodomethyl)tetrahydrofuran-3-ol

To a solution of (2S)-pent-4-ene-1,2-diol (6.5 g, 63.64 mmol) in acetonitrile (100 mL) was added sodium hydrogencarbonate (16 g, 190.93 mmol) at 20° C., the mixture was stirred at 20° C. for 10 min; then iodine (48.5 g, 190.93 mmol) was added at 0° C. After stirred at 25° C. for 110 min, the reaction mixture was quenched by addition of saturated aqueous sodium thiosulfate (400 mL) and extracted with ethyl acetate (100 mL×3). The combined organic layers were washed with brine (50 mL), dried over anhydrous sodium sulfate, filtered and the filtrate was concentrated under reduced pressure to give a residue. The residue was purified by column chromatography to afford the title compound. ¹H NMR (400 MHz, CD₃OD) δ=4.61-4.49 (m, 1H), 4.29-4.15 (m, 1H), 4.15-4.01 (m, 1H), 3.88-3.78 (m, 1H), 3.33-3.26 (m, 2H), 2.23-2.11 (m, 1H), 1.85-1.76 (m, 1H).

### Step 5: (4-hydroxytetrahydrofuran-2-yl)methyl 4-nitrobenzoate

To a solution of (3S)-5-(iodomethyl)tetrahydrofuran-3-ol (9.8 g, 42.98 mmol) in dimethyl sulfoxide (50 mL) was added potassium; 4-nitrobenzoate (6.6 g, 32.23 mmol) and 18-crown-6 (8.5 g, 32.23 mmol) at 20° C. After stirred at 90° C. for 24 h, the reaction mixture was diluted with water (200 mL) and extracted with dichloromethane (100 mL×3). The combined organic layers were washed with brine (100 mL), dried over anhydrous sodium sulfate, filtered and the filtrate was concentrated under reduced pressure to give a residue. The residue was purified by column chromatography to afford the title compound. ¹H NMR (400 MHz, CD₃OD) δ=8.36-8.20 (m, 4H), 4.65-4.47 (m, 3H), 4.40-4.30 (m, 1H), 4.08-4.03 (m, 1H), 3.88-3.81 (m, 1H), 2.13 (dd, J=13.2 Hz, 6.4 Hz, 1H), 1.96-1.85 (m, 1H).

### Step 6: (4-oxotetrahydrofuran-2-yl)methyl 4-nitrobenzoate

To a solution of ((4S)-4-hydroxytetrahydrofuran-2-yl)methyl 4-nitrobenzoate (7.4 g, 27.7 mmol) in 1,2-dichloroethane (100 mL) was added dess-martin periodinane (8.8 g, 20.77 mmol) at 0° C. After stirred at 20° C. for 12 h, the mixture was quenched by addition of saturated aqueous sodium thiosulfate (60 mL) and extracted with ethyl acetate (50 mL×3). The combined organic layers were washed with brine (40 mL), dried over anhydrous sodium sulfate, filtered and the filtrate was concentrated under reduced pressure. The residue was purified by column chromatography to afford the title compound. ¹H NMR (400 MHz, CDCl₃) δ=8.36-8.20 (m, 4H), 4.65-4.47 (m, 3H), 4.40-4.30 (m, 1H), 4.08-4.03 (m, 1H), 2.21 (s, 1H), 2.13 (dd, J=13.2 Hz, 6.4 Hz, 1H).

### Step 7: (4-(7-chloro-2H-benzo[b][1,4]oxazin-4(3H)-yl)tetrahydrofuran-2-yl)methyl 4-nitrobenzoate

To a solution of (4-oxotetrahydrofuran-2-yl)methyl 4-nitrobenzoate (5.6 g, 21.11 mmol) in acetonitrile (20 mL) was added 7-chloro-3,4-dihydro-2H-1,4-benzoxazine (3.9 g, 23.23 mmol), sodium triacetoxyborohydride (9 g, 42.23 mmol) and trifluoroacetic acid (2.4 g, 21.11 mmol) at 20° C. After stirred at 20° C. for 12 h, the mixture was diluted with water (100 mL) and extracted with ethyl acetate (50 mL×3). The combined organic layers were washed with brine (50 mL), dried over anhydrous sodium sulfate, filtered and the filtrate was concentrated under reduced pressure. The residue was purified by column chromatography to afford the title compound. LCMS R_{T}=1.750 min, m/z=419.1 [M+H]⁺.

### Step 8: (4-(7-chloro-2H-benzo[b][1,4]oxazin-4(3H)-yl)tetrahydrofuran-2-yl)methanol

To a solution of (4-(7-chloro-2H-benzo[b][1,4]oxazin-4(3H)-yl tetrahydrofuran-2-yl)methyl 4-nitrobenzoate (5.1 g, 12.18 mmol) in methanol (10 mL) and water (1 mL) was added sodium hydroxide (974 mg, 24.35 mmol) at 20° C. After stirred at 20° C. for 1 h, The reaction mixture was diluted with water (20 mL) and extracted with ethyl acetate (10 mL×3). The combined organic layers were washed with brine (10 mL), dried over anhydrous sodium sulfate, filtered and the filtrate was concentrated under reduced pressure to give a residue. The residue was purified by column chromatography to afford the title compound. LCMS R_{T}=1.308 min, m/z=270.2 [M+H]⁺.

### Step 9

The racemate was separated by SFC to give first eluting fraction (227.1, Rt=1.695 min; LCMS R_{T}=1.308 min, m/z=270.2 [M+H]+), second eluting fraction (227.2, Rₜ=2.162 min; LCMS R_{T}=0.492 min, m/z=270.0 [M+H]), third eluting fraction (227.3, Rt=2.057 min; LCMS R_{T}=0.491 min, m/z=270.1 [M+H]+), and fourth eluting fraction (227.4, Rt=2.275 min; LCMS R_{T}=0.496 min, m/z=270.1 [M+H]+). (*absolute configuration was not determined.) Step 10: 7-chloro-4-(5-(((tetrahydro-2H-pyran-2-yl)oxy)methyl)tetrahydrofuran-3-yl)-3,4-dihydro-2H-benzo[b][1,4]oxazine

To a solution of 227.1 (210 mg, 0.78 mmol) in acetone (0.5 mL) was added pyridine 4-methylbenzenesulfonate (78 mg, 0.31 mmol) and 3,4-dihydro-2H-pyran (294 mg, 3.50 mmol) at 25° C. After stirred at 45° C. for 0.5 h, the reaction mixture was concentrated under reduced pressure. The residue was purified by column chromatography to afford the title compound. LCMS R_{T}=1.013, m/z=354.7 [M+H]⁺.

### Step 11: 5-bromo-7-chloro-4-(5-(((tetrahydro-2H-pyran-2-yl)oxy)methyl)tetrahydrofuran-3-yl)-3,4-dihydro-2H-benzo[b][1,4]oxazine

To a solution of 7-chloro-4-[5-(tetrahydropyran-2-yloxymethyl)tetrahydrofuran-3-yl]-2,3-dihydro-1,4-benzoxazine (280 mg, 0.79 mmol) in DMF (1 mL) was added 1-bromopyrrolidine-2,5-dione (7 mg, 0.04 mmol) at 0° C. After stirred at 25° C. for 1 h, the reaction mixture was diluted with water (30 mL) and extracted with ethyl acetate (10 mL×4). The combined organic layers dried over anhydrous sodium sulfate, filtered and the filtrate was concentrated under reduced pressure. The residue was purified by column chromatography to afford the title compound. LCMS R_{T}=0.642, m/z=434.0 [M+H]⁺.

### Step 12: 5-(2-(((tert-butyldimethylsilyl)oxy)methyl)thieno[3,2-b]pyridin-7-yl)-7-chloro-4-(5-(((tetrahydro-2H-pyran-2-yl)oxy)methyl)tetrahydrofuran-3-yl)-3,4-dihydro-2H-benzo[b][1,4]oxazine

To a solution of 5-bromo-7-chloro-4-(5-(((tetrahydro-2H-pyran-2-yl)oxy)methyl)tetrahydrofuran-3-yl)-3,4-dihydro-2H-benzo[b][1,4]oxazine (220 mg, 0.51 mmol) in dioxane (4 mL) and water (0.4 mL) was added (2-(((tert-butyldimethylsilyl)oxy)methyl)thieno[3,2-b]pyridin-7-yl)boronic acid (131 mg, 0.41 mmol), palladium dichloride[1,1′-bis (diphenylphosphine) ferrocene] (37 mg, 0.05 mmol) and cesium carbonate (331 mg, 1.02 mmol) at 25° C. After stirred at 100° C. for 3 h, the reaction mixture was concentrated under reduced pressure. The residue was purified by column chromatography to afford the title compound. LCMS R_{T}=1.123, m/z=631.3 [M+H]⁺.

### Step 13: (7-(7-chloro-4-(5-(((tetrahydro-2H-pyran-2-yl)oxy)methyl)tetrahydrofuran-3-yl)-3,4-dihydro-2H-benzo[b][1,4]oxazin-5-yl)thieno[3,2-b]pyridin-2-yl)methanol

To a solution of 5-(2-(((tert-butyldimethylsilyl)oxy)methyl)thieno[3,2-b]pyridin-7-yl)-7-chloro-4-(5-(((tetrahydro-2H-pyran-2-yl)oxy)methyl)tetrahydrofuran-3-yl)-3,4-dihydro-2H-benzo[b][1,4]oxazine (150 mg, 0.24 mmol) in THF (1 mL) was added tetrabutylammonium fluoride (1 M in THF, 0.2 mL) at 25° C. After stirred at 25° C. for 0.5 h, the reaction mixture was diluted with water (20 mL) and extracted with ethyl acetate (15 mL×4). The combined organic layers dried over anhydrous sodium sulfate, filtered and the filtrate was concentrated under reduced pressure. The residue was purified by column chromatography to afford the title compound. LCMS R_{T}=0.863, m/z=517.2 [M+H]⁺.

### Step 14

Example 227a was prepared from (7-(7-chloro-4-(5-(((tetrahydro-2H-pyran-2-yl)oxy)methyl)tetrahydrofuran-3-yl)-3,4-dihydro-2H-benzo[b][1,4]oxazin-5-yl)thieno[3,2-b]pyridin-2-yl)methanol and succinimide, following the procedure described in the synthesis of Example 108. The product was purified by preparative HPLC. ¹H NMR (400 MHz, CD₃OD) δ=8.65-8.72 (m, 1H), 7.41-7.52 (m, 2H), 6.92-6.97 (m, 1H), 6.88 (d, J=2.4 Hz, 1H), 4.95 (s, 2H), 4.09-4.27 (m, 2H), 3.47-3.54 (m, 2H), 3.31-3.45 (m, 7H), 2.75 (s, 4H), 1.41-1.56 (m, 1H). LCMS R_{T}=1.510, m/z=514.3[M+H]⁺.

Example 227b was prepared from 227.2, following the same procedures in the synthesis of Example 227a. The final product was purified by preparative HPLC. ¹H NMR (400 MHz, CD₃OD) δ=8.69 (d, J=4.8 Hz, 1H), 7.49 (s, 1H), 7.45 (d, J=4.8 Hz, 1H), 6.95 (d, J=2.4 Hz, 1H), 6.88 (d, J=2.4 Hz, 1H), 4.95 (s, 2H), 4.19 (d, J=12.8 Hz, 2H), 3.56-3.47 (m, 2H), 3.45-3.31 (m, 1H), 3.45-3.31 (m, 7H), 2.75 (s, 4H). LCMS R_{T}=1.508 min, m/z=514.3 [M+H]⁺.

Example 227c was prepared from 227.3, following the same procedures in the synthesis of Example 227a. The final product was purified by preparative HPLC. ¹H NMR (400 MHz, CD₃OD) δ=8.72-8.64 (m, 1H), 7.53-7.46 (m, 1H), 7.44-7.40 (m, 1H), 6.95 (d, J=2.4 Hz, 1H), 6.87 (d, J=2.4 Hz, 1H), 4.97-4.94 (m, 2H), 4.24-4.15 (m, 2H), 3.93-3.83 (m, 1H), 3.48 (dd, J=8.4 Hz, 6.4 Hz, 1H), 3.37-3.32 (m, 3H), 3.30-3.25 (m, 2H), 3.19-3.11 (m, 1H), 3.11-3.04 (m, 1H), 2.78-2.72 (m, 4H), 1.82-1.62 (m, 1H). LCMS R_{T}=1.503 min, m/z=514.3 [M+H]⁺.

Example 227d was prepared from 227.4, following the same procedures in the synthesis of Example 227a. The final product was purified by preparative HPLC. ¹H NMR (400 MHz, CD₃OD) δ=8.72-8.64 (m, 1H), 7.53-7.46 (m, 1H), 7.44-7.40 (m, 1H), 6.95 (d, J=2.4 Hz, 1H), 6.87 (d, J=2.4 Hz, 1H), 4.97-4.94 (m, 2H), 4.24-4.15 (m, 2H), 3.93-3.83 (m, 1H), 3.48 (dd, J=6.4, 8.4 Hz, 1H), 3.37-3.32 (m, 3H), 3.30-3.25 (m, 2H), 3.19-3.11 (m, 1H), 3.11-3.04 (m, 1H), 2.78-2.72 (m, 4H), 1.82-1.62 (m, 1H). LCMS R_{T}=1.497 min, m/z=514.3 [M+H]⁺

### Example 228: 1-((7-(6-chloro-2-oxo-1-(1H-pyrazol-4-yl)-1,2,3,4-tetrahydroquinolin-8-yl)thieno[3,2-b]pyridin-2-yl)methyl)pyrrolidine-2,5-dione

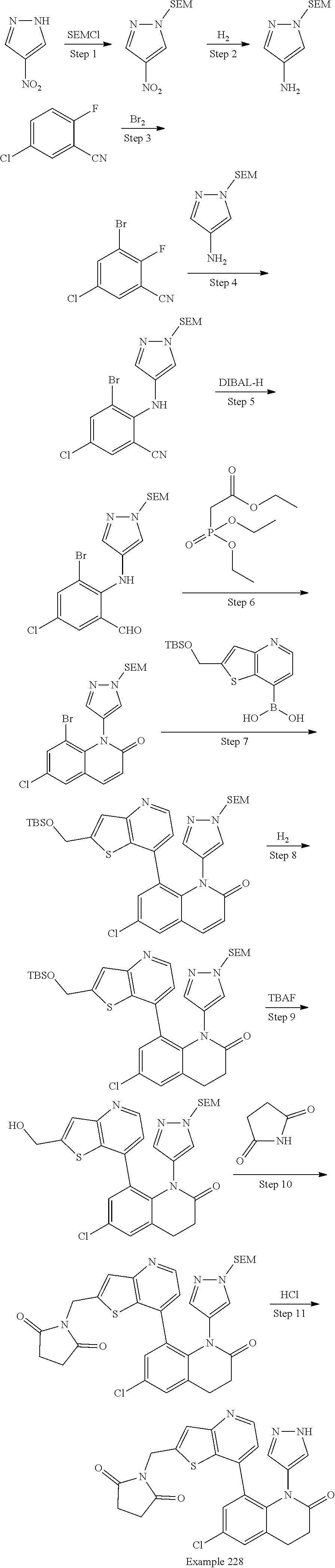

### Step 1: 4-nitro-1-((2-(trimethylsilyl)ethoxy)methyl)-1H-pyrazole

To a solution of 4-nitro-1H-pyrazole (15 g, 132.66 mmol) in THF (150 mL) was added sodium hydride (8 g, 198.98 mmol, 60% purity) at 0° C. After stirred at 20° C. for 30 min, (2-(chloromethoxy)ethyl)trimethylsilane (28 mL, 159.19 mmol) was added. After stirred at 20° C. for 16 h under nitrogen atmosphere, the reaction mixture was quenched by addition of water (30 mL) and extracted with ethyl acetate (50 mL×2). The combined organic layers were washed with brine (50 mL), dried over anhydrous sodium sulfate, filtered and the filtrate was concentrated under reduced pressure. The residue was purified by column chromatography to afford the title compound. ¹H NMR (400 MHz, CDCl₃) δ=8.40-8.25 (m, 1H), 8.18-8.03 (m, 1H), 5.49-5.42 (m, 2H), 3.69-3.57 (m, 2H), 0.99-0.90 (m, 2H), 0.02-0.06 (m, 9H).

### Step 2: 1-((2-(trimethylsilyl)ethoxy)methyl)-1H-pyrazol-4-amine

To a solution of 4-nitro-1-((2-(trimethylsilyl)ethoxy)methyl)-1H-pyrazole (55.8 g, 229.15 mmol) in ethyl acetate (100 mL) was added 10% palladium on carbon (12 g, 22.92 mmol). After stirred at 20° C. for 8 h under hydrogen atmosphere, the reaction mixture was filtered and the filtrate was concentrated under reduced pressure to afford the title compound. It was used directly and without further purification in the next step. ¹H NMR (400 MHz, CDCl₃) δ=7.25-7.12 (m, 2H), 5.36-5.37 (m, 2H), 3.56-3.46 (m, 2H), 2.96-2.29 (m, 2H), 0.93-0.84 (m, 2H), 0.00-0.08 (m, 9H).

### Step 3: 3-bromo-5-chloro-2-fluorobenzonitrile

To a solution of lithium magnesium 2,2,6,6-tetramethylpiperidin-1-ide dichloride (116 mL, 1M in THF) was added 5-chloro-2-fluorobenzonitrile (15 g, 96.43 mmol) in THF (150 mL) at −65° C. After stirred at 0° C. for 1 h under nitrogen atmosphere, bromine (15 mL, 289.29 mmol) was added. After stirred at −65° C. for 1 h under nitrogen atmosphere, the reaction mixture was quenched by addition of saturated ammonium chloride (50 mL) and extracted with ethyl acetate (50 mL×2). The combined organic layers were washed with brine (50 mL), dried over anhydrous sodium sulfate, filtered and the filtrate was concentrated under reduced pressure. The residue was purified by column chromatography to afford the title compound. ¹H NMR (400 MHz, CDCl₃) δ=7.87-7.78 (m, 1H), 7.62-7.52 (m, 1H).

### Step 4: 3-bromo-5-chloro-2-((1-((2-(trimethylsilyl)ethoxy)methyl)-1H-pyrazol-4-yl)amino)benzonitrile

To a solution of 3-bromo-5-chloro-2-fluorobenzonitrile (15 g, 63.98 mmol) in (methylsulfinyl)methane (150 mL) was added N-ethyl-N-isopropylpropan-2-amine (22.3 mL, 127.96 mmol) and 1-((2-(trimethylsilyl)ethoxy)methyl)-1H-pyrazol-4-amine (15 g, 70.38 mmol). After stirred at 110° C. for 16 h under nitrogen atmosphere, the reaction mixture was diluted with water (50 mL) and extracted with ethyl acetate (50 mL×2). The combined organic layers were washed with brine (20 mL), dried over anhydrous sodium sulfate, filtered and the filtrate was concentrated under reduced pressure. The residue was purified by column chromatography to afford the title compound. ¹H NMR (400 MHz, CDCl₃) δ=7.68-7.64 (m, 1H), 7.60 (d, J=2.4 Hz, 1H), 7.51-7.47 (m, 1H), 7.40-7.35 (m, 1H), 6.03-5.98 (m, 1H), 5.46-5.40 (m, 2H), 3.65-3.58 (m, 2H), 0.97-0.91 (m, 2H), 0.04-0.03 (m, 8H).

### Step 5: 3-bromo-5-chloro-2-((1-((2-(trimethylsilyl)ethoxy)methyl)-1H-pyrazol-4-yl)amino)benzaldehyde

To a solution of 3-bromo-5-chloro-2-((1-((2-(trimethylsilyl)ethoxy)methyl)-1H-pyrazol-4-yl)amino)benzonitrile (12.8 g, 29.92 mmol) in dichloromethane (100 mL) was added diisobutyl aluminum hydride (74.8 mL, 1M in THF) at −65° C. After stirred at −65° C. for 1 h under nitrogen atmosphere, the reaction mixture was quenched by addition of saturated sodium hydroxide (10 mL) and extracted with dichloromethane (20 mL×2). The combined organic layers were washed with brine (20 mL), dried over anhydrous sodium sulfate, filtered and the filtrate was concentrated under reduced pressure. The residue was purified by column chromatography to afford the title compound. ¹H NMR (400 MHz, CDCl₃) δ=9.91-9.86 (m, 1H), 8.71-8.61 (m, 1H), 7.72-7.63 (m, 1H), 7.61-7.54 (m, 1H), 7.40-7.32 (m, 2H), 5.41-5.34 (m, 2H), 3.58-3.50 (m, 2H), 0.95-0.90 (m, 2H), 0.03-0.03 (m, 9H).

### Step 6: 8-bromo-6-chloro-1-(1-((2-(trimethylsilyl)ethoxy)methyl)-1H-pyrazol-4-yl)quinolin-2(1H)-one

To a solution of 3-bromo-5-chloro-2-((1-((2-(trimethylsilyl)ethoxy)methyl)-1H-pyrazol-4-yl)amino)benzaldehyde (3.6 g, 8.36 mmol) in ethyl alcohol (15 mL) was added ethyl 2-(diethoxyphosphoryl)acetate (3.75 g, 16.71 mmol) and potassium carbonate (3.46 g, 25.07 mmol). After stirred at 100° C. for 8 h under nitrogen atmosphere, the reaction mixture was diluted with water (50 mL) and extracted with ethyl acetate (50 mL×2). The combined organic layers were washed with brine (20 mL), dried over anhydrous sodium sulfate, filtered and the filtrate was concentrated under reduced pressure. The residue was purified by column chromatography to afford the title compound. ¹H NMR (400 MHz, CDCl₃) δ=7.75-7.67 (m, 2H), 7.63-7.58 (m, 1H), 7.54-7.47 (m, 2H), 6.82-6.76 (m, 1H), 5.52-5.45 (m, 2H), 3.71-3.61 (m, 2H), 1.02-0.90 (m, 2H), 0.04-0.03 (m, 9H). LCMS R_{T}=1.071 min, m/z=456.0 [M+H]⁺.

### Step 7: 8-(2-(((tert-butyldimethylsilyl)oxy)methyl)thieno[3,2-b]pyridin-7-yl)-6-chloro-1-(1-((2-(trimethylsilyl)ethoxy)methyl)-1H-pyrazol-4-yl)quinolin-2(1H)-one

To a solution of 8-bromo-6-chloro-1-(1-((2-(trimethylsilyl)ethoxy)methyl)-1H-pyrazol-4-yl)quinolin-2(1H)-one (500 mg, 1.10 mmol) in dioxane (5 mL) and water (0.5 mL) was added (2-(((tert-butyldimethylsilyl)oxy)methyl)thieno[3,2-b]pyridin-7-yl)boronic acid (533 mg, 1.65 mmol), cesium carbonate (1.07 g, 3.30 mmol) and 1,1-bis(diphenylphosphorus) ferrocene palladium chloride⋅dichloromethane (90 mg, 0.11 mmol). After stirred at 90° C. for 16 h under nitrogen atmosphere, the reaction mixture was filtered and the filtrate was diluted with water (20 mL) and extracted with ethyl acetate (20 mL×2). The combined organic layers were washed with brine (20 mL), dried over anhydrous sodium sulfate, filtered and the filtrate was concentrated under reduced pressure. The residue was purified by column chromatography to afford the title compound. LCMS R_{T}=2.656 min, m/z=653.2 [M+H]⁺.

### Step 8: 8-(2-(((tert-butyldimethylsilyl)oxy)methyl)thieno[3,2-b]pyridin-7-yl)-6-chloro-1-(1-((2-(trimethylsilyl)ethoxy)methyl)-1H-pyrazol-4-yl)-3,4-dihydroquinolin-2(1H)-one

To a solution of 8-(2-(((tert-butyldimethylsilyl)oxy)methyl)thieno[3,2-b]pyridin-7-yl)-6-chloro-1-(1-((2-(trimethylsilyl)ethoxy)methyl)-1H-pyrazol-4-yl)quinolin-2(1H)-one (500 mg, 0.77 mmol) in acetic acid (5 mL) was added platinum dioxide (17 mg, 0.08 mmol). After stirred at 40° C. for 24 h under hydrogen atmosphere, the reaction mixture was quenched by addition of saturated sodium bicarbonate (10 mL) and extracted with ethyl acetate (20 mL×2). The combined organic layers were washed with brine (20 mL), dried over anhydrous sodium sulfate, filtered and the filtrate was concentrated under reduced pressure. The residue was purified by preparative HPLC to afford the title compound. LCMS R_{T}=2.693 min, m/z=655.2 [M+H]⁺.

### Step 9: 6-chloro-8-(2-(hydroxymethyl)thieno[3,2-b]pyridin-7-yl)-1-(1-((2-(trimethylsilyl)ethoxy)methyl)-1H-pyrazol-4-yl)-3,4-dihydroquinolin-2(1H)-one

To a solution of 8-(2-(((tert-butyldimethylsilyl)oxy)methyl)thieno[3,2-b]pyridin-7-yl)-6-chloro-1-(1-((2-(trimethylsilyl)ethoxy)methyl)-1H-pyrazol-4-yl)-3,4-dihydroquinolin-2(1H)-one (115 mg, 0.18 mmol) in THF (1 mL) was added tetrabutylammonium fluoride (0.18 mL, 1M in THF). After stirred at 20° C. for 0.5 h, the reaction mixture was diluted with water (20 mL) and extracted with ethyl acetate (20 mL×2). The combined organic layers were washed with brine (20 mL), dried over anhydrous sodium sulfate, filtered and the filtrate was concentrated under reduced pressure. The residue was purified by preparative TLC to afford the title compound. LCMS R_{T}=1.633 min, m/z=541.2 [M+H]⁺.

### Step 10: 1-((7-(6-chloro-2-oxo-1-(1-((2-(trimethylsilyl)ethoxy)methyl)-1H-pyrazol-4-yl)-1,2,3,4-tetrahydroquinolin-8-yl)thieno[3,2-b]pyridin-2-yl)methyl)pyrrolidine-2,5-dione

To a solution of 6-chloro-8-(2-(hydroxymethyl)thieno[3,2-b]pyridin-7-yl)-1-(1-((2-(trimethylsilyl)ethoxy)methyl)-1H-pyrazol-4-yl)-3,4-dihydroquinolin-2(1H)-one in toluene (1 mL) was added succinimide (12 mg, 0.12 mmol), tributylphosphane (97 mg, 0.48 mmol) and (E)-1,1′-(diazene-1,2-diyl)bis(2-methylpropan-1-one) (41 mg, 0.24 mmol). After stirred at 90° C. for 1 h under nitrogen atmosphere, the reaction mixture was filtered and the filtrate was concentrated under reduced pressure. The residue was purified by preparative TLC to afford the title compound. LCMS R_{T}=2.087 min, m/z=622.4 [M+H]⁺.

### Step 11

To a solution of 1-((7-(6-chloro-2-oxo-1-(1-((2-(trimethylsilyl)ethoxy)methyl)-1H-pyrazol-4-yl)-1,2,3,4-tetrahydroquinolin-8-yl)thieno[3,2-b]pyridin-2-yl)methyl)pyrrolidine-2,5-dione (20 mg, 0.03 mmol) was dissolved in hydrochloric acid (1 mL, 4 M in dioxane). After stirred at 20 C for 2 h, the reaction mixture was filtered and the filtrate was concentrated under reduced pressure. The residue was purified by preparative HPLC to afford Example 228. ¹H NMR (400 MHz, CD₃OD) δ=12.45-11.84 (m, 1H), 8.45-8.34 (m, 1H), 7.66-7.52 (m, 1H), 7.46-7.34 (m, 1H), 7.30-7.20 (m, 1H), 7.01-6.90 (m, 1H), 6.86-6.43 (m, 2H), 4.93-4.74 (m, 2H), 3.11-2.97 (m, 2H), 2.81-2.63 (m, 6H). LCMS R_{T}=1.093 min, m/z=492.3 [M+H]⁺.

### Example 229a and 229b: (R)-1-((7-(6′-chloro-3′,4′-dihydro-2′H-spiro[morpholine-2,1′-naphthalen]-8′-yl)thieno[3,2-b]pyridin-2-yl)methyl)pyrrolidine-2,5-dione, formic acid salt and (S)-1-((7-(6′-chloro-3′,4′-dihydro-2′H-spiro[morpholine-2,1′-naphthalen]-8′-yl)thieno[3,2-b]pyridin-2-yl)methyl)pyrrolidine-2,5-dione, formic acid salt

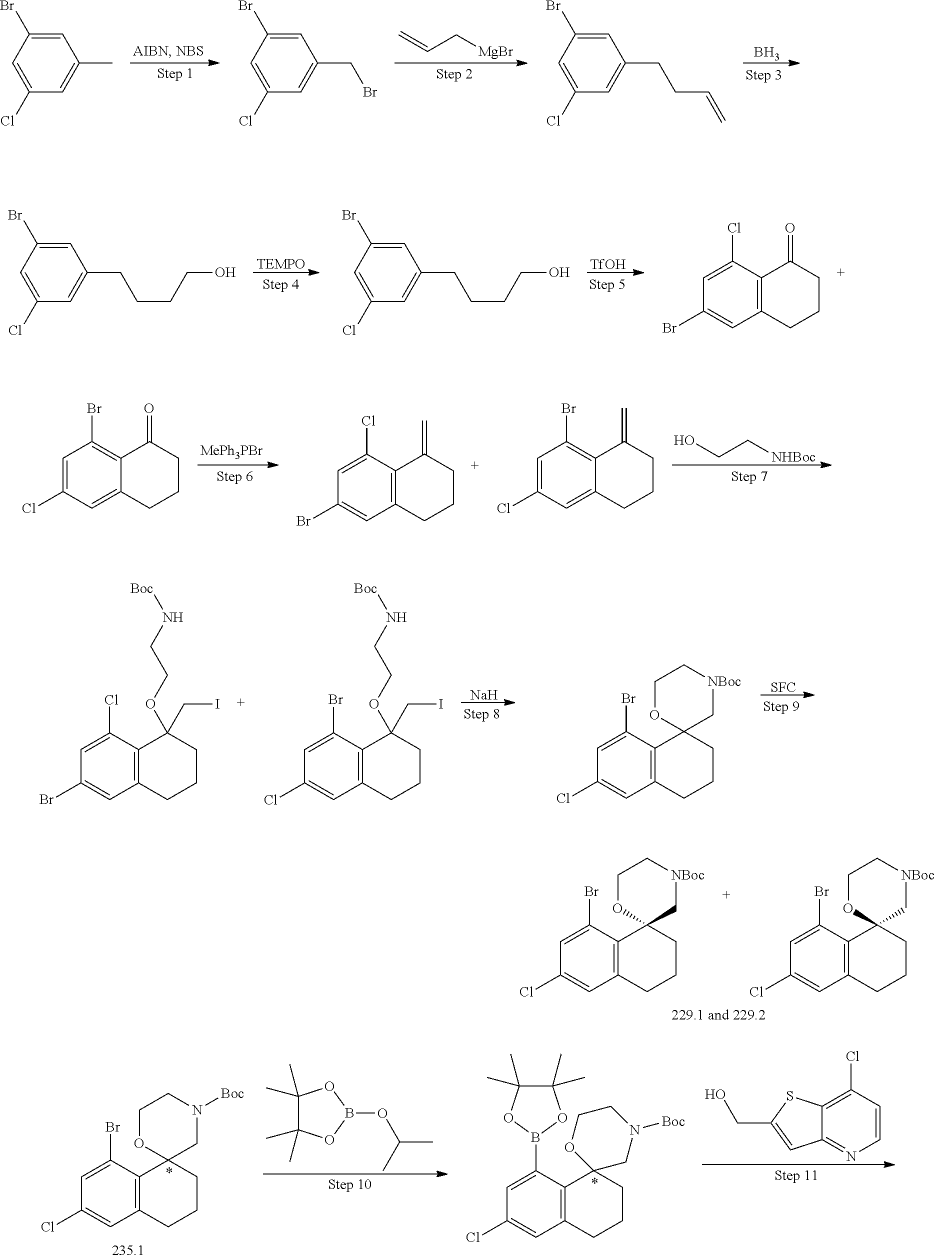

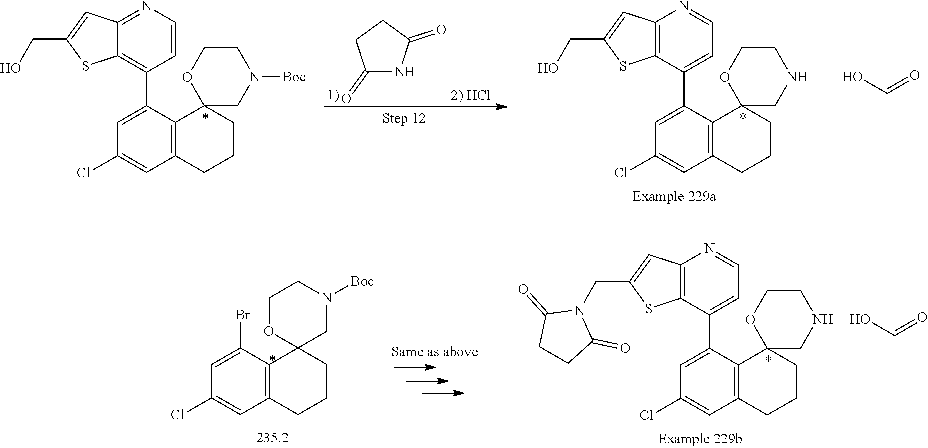

### Step 1: 1-bromo-3-(bromomethyl)-5-chlorobenzene

To the solution of 1-bromo-3-chloro-5-methylbenzene (30 g, 146 mmol) in dichloromethane (20 mL) was added N-bromosuccinimide (28.6 g, 160.6 mmol) and azodiisobutyronitrile (2.4 g, 14.6 mmol) at 20° C. After stirred at 20° C. for 12 h, the reaction mixture was filtered and the filtrate was concentrated under reduced pressure. The residue was purified by column chromatography to afford the title compound. ¹H NMR (400 MHz, CDCl₃) δ=7.36 (d, J=1.6, 7.6 Hz, 2H), 7.26-7.23 (m, 1H), 4.29 (s, 2H).

### Step 2: 1-bromo-3-(but-3-en-1-yl)-5-chlorobenzene

To the solution of 1-bromo-3-(bromomethyl)-5-chlorobenzene (30 g, 105.5 mmol) in THF (50 mL) was dropwise added allylmagnesium bromide (63.3 mL, 1 M in THF) at −78° C. for 1 h. After stirred at 0° C. for 1 h under nitrogen atmosphere, the reaction mixture was quenched by addition of saturated ammonium chloride solution (50 mL) and was extracted with ethyl acetate (50 mL×3). The combined organic layers were washed with brine (100 mL), dried over anhydrous sodium sulfate and concentrated under reduced pressure. The residue was purified by column chromatography to afford the title compound. ¹H NMR (400 MHz, CDCl₃) δ=7.37-7.33 (m, 1H), 7.24-7.22 (m, 1H), 7.13-7.10 (in, 1H), 593-5.69 (m, 1H), 5.07 (q, J=1.6 Hz, 2H), 2.73-2.60 (m, 2H), 2.40-2.33 (in, 2H).

### Step 3: 4-(3-bromo-5-chlorophenyl)butan-1-ol

To the solution of 1-bromo-3-(but-3-en-1-yl)-5-chlorobenzene (24.2 g, 98.6 mmol) in THF (80 mL) was added borane (3.05 mL, 10 M in dimethylsulfide) at 0° C. After stirred at 20° C. for 1 h under nitrogen atmosphere, the reaction mixture was added sodium hydroxide solution (4.07 mL, 3 M in water) and hydrogen peroxide (11.74 mL, 30% purity) at 20° C. After stirred at 20° C. for 1 h under nitrogen atmosphere, the reaction mixture was quenched by addition of saturated sodium thiosulfate solution (100 mL) and was extracted with ethyl acetate (100 mL×3). The combined organic layers were washed with brine (200 mL), dried over anhydrous sodium sulfate and concentrated under reduced pressure. The residue was purified by column chromatography to afford the title compound. ¹H NMR (400 MHz, CDCl₃) δ=7.40-7.30 (m, 1H), 7.25-7.18 (m, 1H), 7.14-7.07 (m, 1H), 3.73-3.62 (m, 2H), 2.66-2.55 (m, 2H), 1.73-1.54 (m, 4H).

### Step 4: 4-(3-bromo-5-chlorophenyl)butanoic acid

To a solution of 4-(3-bromo-5-chlorophenyl)butan-1-ol (11.1 g, 42.2 mmol) in acetonitrile (15 mL) and water (9 mL) was added 2,2,6,6-tetramethylpiperidine (664 mg, 4.2 mmol), potassium bicarbonate (16.9 g, 168.92 mmol) and sodium hypochlorite solution (16.34 mL, 10% purity) at 20° C. After stirred at 20° C. for 1 h, the reaction mixture was adjusted to pH 10 by sodium hydroxide solution and extracted with ethyl acetate (100 mL×3). The combined organic layers were washed with brine (200 mL), then the combined water layers was adjusted to pH 2 by hydrochloric acid and extracted with ethyl acetate (100 mL×3). The combined organic layers were washed with brine (200 mL), dried over anhydrous sodium sulfate and concentrated under reduced pressure to afford the title compound. It was used directly and without further purification in the next step. ¹H NMR (400 MHz, CDCl₃) δ=10.19-9.70 (m, 1H), 7.38-7.35 (m, 1H), 7.26-7.22 (m, 1H), 7.14-7.10 (m, 1H), 2.68-2.59 (m, 2H), 2.44-2.37 (m, 2H), 2.01-1.90 (m, 2H).

### Step 5: 6-bromo-8-chloro-3,4-dihydronaphthalen-1(2H)-one and 8-bromo-6-chloro-3,4-dihydronaphthalen-1(2H)-one

To the mixture of 4-(3-bromo-5-chlorophenyl)butanoic acid (18.8 g, 67.74 mmol) was dissolved in trifluoromethanesulfonic acid (100 mL). After stirred at 0° C. for 1 h and stirred at 20° C. for 2 h, the reaction mixture was quenched by addition of saturated sodium bicarbonate solution (100 mL) and was extracted with ethyl acetate (100 mL×3). The combined organic layers were washed with brine (200 mL), dried over anhydrous sodium sulfate and concentrated under reduced pressure. The residue was purified by column chromatography to afford the title compound. ¹H NMR (400 MHz, CDCl₃) δ=7.62-7.55 (m, 1H), 7.54-7.47 (m, 1H), 7.39-7.31 (m, 1H), 7.26-7.20 (m, 1H), 2.99-2.92 (m, 4H), 2.74-2.65 (m, 4H), 2.15-2.06 (m, 4H).

### Step 6: 6-bromo-8-chloro-1-methylene-1,2,3,4-tetrahydronaphthalene and 8-bromo-6-chloro-1-methylene-1,2,3,4-tetrahydronaphthalene

To a solution of methyltriphenylphosphonium bromide (5.2 g, 14.45 mmol) in THF (3 mL) was added potassium tert-butoxide (14.45 mL, 1 M in THF) at 0° C. After stirred at 0° C. for 10 min under nitrogen atmosphere, the reaction mixture was added the solution of 6-bromo-8-chloro-3,4-dihydronaphthalen-1(2H)-one (1.3 g, 4.82 mmol) and 8-bromo-6-chloro-3,4-dihydronaphthalen-1(2H)-one (1.3 g, 4.82 mmol) in THF (3 mL) at 0° C. After stirred at 20° C. for 110 min under nitrogen atmosphere, the reaction mixture was extracted with ethyl acetate (100 mL×3). The combined organic layers were washed with brine (200 mL), dried over anhydrous sodium sulfate and concentrated under reduced pressure. The residue was purified by column chromatography to afford the title compound. ¹H NMR (400 MHz, CDCl₃) δ=7.52-7.47 (m, 1H), 7.44-7.38 (m, 1H), 7.21-7.16 (m, 1H), 7.10-7.04 (m, 1H), 5.75-5.61 (m, 2H), 5.44-5.35 (m, 2H), 2.78-2.65 (m, 4H), 2.54-2.44 (m, 4H), 1.92-1.80 (m, 4H).

### Step 7: tert-butyl (2-((6-bromo-8-chloro-1-(iodomethyl)-1,2,3,4-tetrahydronaphthalen-1-yl)oxy)ethyl)carbamate compound with tert-butyl (2-((8-bromo-6-chloro-1-(iodomethyl)-1,2,3,4-tetrahydronaphthalen-1-yl)oxy)ethyl)carbamate

To a solution of 6-bromo-8-chloro-1-methylene-1,2,3,4-tetrahydronaphthalene (0.9 g, 3.61 mmol) and 8-bromo-6-chloro-1-methylene-1,2,3,4-tetrahydronaphthalene (0.9 g, 3.61 mmol) in acetonitrile (30 mL) was added tert-butyl (2-hydroxyethyl)carbamate (1.3 g, 7.94 mmol) and 1-iodopyrrolidine-2,5-dione (1.8 g, 7.94 mmol). After stirred at 20° C. for 2 h, the reaction mixture was filtered and the filtrate was concentrated under reduced pressure. The residue was purified by column chromatography to afford the title compound as a mixture of both cis and trans isomers. LCMS R_{T}=3.005 min, m/z=568.1 [M+Na]⁺.

### Step 8: tert-butyl 6′-bromo-8′-chloro-3′,4′-dihydro-2′H-spiro[morpholine-2,1′-naphthalene]-4-carboxylate compound and tert-butyl 8′-bromo-6′-chloro-3′,4′-dihydro-2′H-spiro[morpholine-2,1′-naphthalene]-4-carboxylate

To a solution of tert-butyl (2-((6-bromo-8-chloro-1-(iodomethyl)-1,2,3,4-tetrahydronaphthalen-1-yl)oxy)ethyl)carbamate compound (1.4 g, 2.53 mmol) and tert-butyl (2-((8-bromo-6-chloro-1-(iodomethyl)-1,2,3,4-tetrahydronaphthalen-1-yl)oxy)ethyl)carbamate (1.4 g, 2.53 mmol) in N,N-dimethylacetamide (30 mL) was added sodium hydrogen (202 mg, 5.07 mmol, 60% purity). After stirred at 20° C. for 1 h under nitrogen atmosphere, the reaction mixture was quenched by addition of water (10 mL) and extracted with ethyl acetate (10 mL×3). The combined organic layers were washed with brine (20 mL), dried over anhydrous sodium sulfate and concentrated under reduced pressure. The residue was purified by column chromatography to afford the title compound as a mixture of both cis and trans isomers. LCMS R_{T}=2.867 min, m/z=362.1 [M+H−56]⁺.

### Step 9: (R)-tert-butyl 8′-bromo-6′-chloro-3′,4′-dihydro-2′H-spiro[morpholine-2,1′-naphthalene]-4-carboxylate and (S)-tert-butyl 8′-bromo-6′-chloro-3′,4′-dihydro-2′H-spiro[morpholine-2,1′-naphthalene]-4-carboxylate

The above mixture was separated by SFC to give first eluding fraction (229.1, Rt=0.793 min, LCMS R_{T}=1.174 min, m/z=361.9 [M+H−56]+) and second eluding fraction (229.2, Rt=0.793 min, LCMS R_{T}=1.174 min, m/z=361.9 [M+H−56]+).

### Step 10: tert-butyl 6′-chloro-8′-(4,4,5,5-tetramethyl-1,3,2-dioxaborolan-2-yl)-3′,4′-dihydro-2′H-spiro[morpholine-2,1′-naphthalene]-4-carboxylate

To a solution of isopropyl magnesium lithium chloride complex (1.88 mL, 1.3 M in THF) was added 229.1 (60 mg, 0.14 mmol) in THF (0.25 mL) at 0° C. under nitrogen atmosphere. After stirred at 0° C. for 0.5 h under nitrogen atmosphere, 2-isopropoxy-4,4,5,5-tetramethyl-1,3,2-dioxaborolane (80 mg, 0.43 mmol) was added at 0° C. under nitrogen atmosphere. After stirred at 20° C. for 0.5 h atmosphere, the reaction mixture was quenched by addition of saturated ammonium chloride and extracted with ethyl acetate (5 mL×3). The combined organic layers were washed with brine (10 mL), dried over anhydrous sodium sulfate and concentrated under reduced pressure to afford the title compound. It was used directly and without further purification in the next step. LCMS R_{T}=2.079 min, m/z=464.0 [M+H]⁺.

### Step 11: tert-butyl 6′-chloro-8′-(2-(hydroxymethyl)thieno[3,2-b]pyridin-7-yl)-3′,4′-dihydro-2′H-spiro[morpholine-2,1′-naphthalene]-4-carboxylate

To a solution of tert-butyl 6′-chloro-8′-(4,4,5,5-tetramethyl-1,3,2-dioxaborolan-2-yl)-3′,4′-dihydro-2′H-spiro[morpholine-2,1′-naphthalene]-4-carboxylate (65 mg 0.14 mmol) in dioxane (0.9 mL) and water (0.1 mL) was added (7-chlorothieno[3,2-b]pyridin-2-yl)methanol (42 mg, 0.21 mmol), cesium carbonate (91 mg, 0.28 mmol) and 1,1-bis(diphenylphosphorus) ferrocene palladium chloride (10 mg, 0.014 mmol). After stirred at 100° C. for 3 h under nitrogen atmosphere, the reaction mixture was filtered and the filtrate was concentrated under reduced pressure. The residue was purified by preparative TLC to afford the title compound. LCMS R_{T}=1.747 min, m/z=501.3 [M+H]⁺.

### Step 12

Example 229a was prepared from tert-butyl 6′-chloro-8′-(2-(hydroxymethyl)thieno[3,2-b]pyridin-7-yl)-3′,4′-dihydro-2′H-spiro[morpholine-2,1′-naphthalene]-4-carboxylate (25 mg, 0.05 mmol) and succinimide, following the procedure described in the synthesis of Example 108. The product was purified by preparative HPLC. ¹H NMR (400 MHz, CD₃OD) δ=8.65-8.57 (m, 1H), 7.54-7.47 (m, 1H), 7.46-7.39 (m, 1H), 7.34-7.26 (m, 1H), 7.05-7.00 (m, 1H), 6.95-6.90 (m, 1H), 4.96 (s, 2H), 3.82-3.70 (m, 1H), 3.62-3.52 (m, 1H), 3.42-3.34 (m, 1H), 3.28-3.07 (m, 1H), 3.00-2.85 (m, 2H), 2.80-2.37 (m, 7H), 2.02-1.51 (m, 3H). LCMS R_{T}=1.002 min, m/z=482.1 [M+H]⁺.

Example 229b was prepared from 229.2, following the procedures in the synthesis of Example 229a. The final product was purified by preparative HPLC. ¹H NMR (400 MHz, CD₃OD) δ=8.65-8.58 (m, 1H), 7.53-7.48 (m, 1H), 7.46-7.40 (m, 1H), 7.35-7.27 (m, 1H), 7.06-7.00 (m, 1H), 6.96-6.90 (m, 1H), 4.95-4.90 (m, 2H), 3.84-3.73 (m, 1H), 3.64-3.54 (m, 1H), 3.47-3.37 (m, 1H), 3.28-3.09 (m, 1H), 3.05-2.86 (m, 2H), 2.83-2.41 (m, 7H), 2.07-1.50 (m, 3H). LCMS R_{T}=1.002 min, m/z=482.2 [M+H]⁺.

### Example 230a and 230b: 1-((7-((S)-4-((R)-3-aminopyrrolidin-1-yl)-7-chlorochroman-5-yl)thieno[3,2-b]pyridin-2-yl)methyl)pyrrolidine-2,5-dione, hydrochloride salt and 1-((7-((R)-4-((R)-3-aminopyrrolidin-1-yl)-7-chlorochroman-5-yl)thieno[3,2-b]pyridin-2-yl)methyl)pyrrolidine-2,5-dione, hydrochloride salt

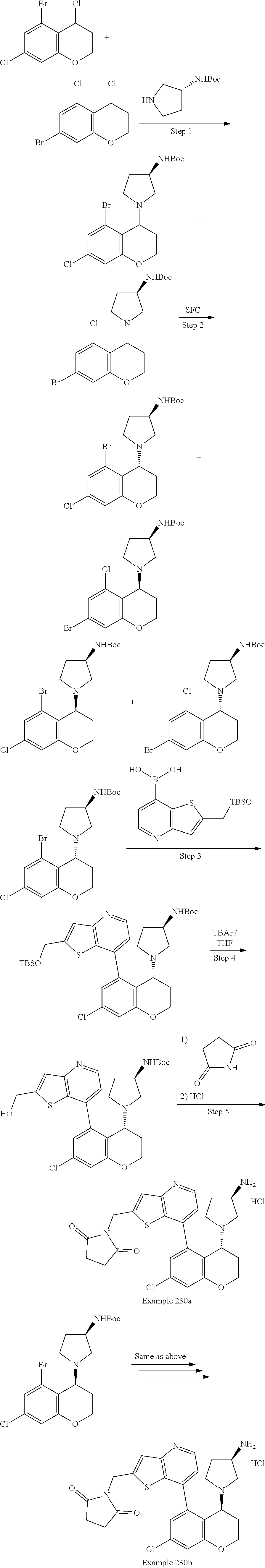

### Step 1: tert-butyl ((3R)-1-(5-bromo-7-chlorochroman-4-yl)pyrrolidin-3-yl)carbamate and tert-butyl ((3R)-1-(7-bromo-5-chlorochroman-4-yl)pyrrolidin-3-yl)carbamate

To a solution of 5-bromo-4,7-dichloro-chromane (2 g, 7.09 mmol) and 7-bromo-4,5-dichloro-chromane (2 g, 7.09 mmol) in acetonitrile (15 mL) was added tert-butyl N-[(3R)-pyrrolidin-3-yl]carbamate (2.64 g, 14.19 mmol), potassium carbonate (1.96 g, 14.19 mmol) and potassium iodide (1.18 g, 7.09 mmol) at 25° C. After stirred at 80° C. for 2 h, the reaction mixture was concentrated under reduced pressure. The residue was purified by preparative HPLC to afford the title compound. LCMS R_{T}=0.810 min, m/z=432.9 [M+2+H]⁺.

### Step 2: tert-butyl ((R)-1-((R)-5-bromo-7-chlorochroman-4-yl)pyrrolidin-3-yl)carbamate, tert-butyl ((R)-1-((S)-7-bromo-5-chlorochroman-4-yl)pyrrolidin-3-yl)carbamate, tert-butyl ((R)-1-((S)-5-bromo-7-chlorochroman-4-yl)pyrrolidin-3-yl)carbamate and tert-butyl ((R)-1-((R)-7-bromo-5-chlorochroman-4-yl)pyrrolidin-3-yl)carbamate

The above mixture was separated by SFC to give first eluting fraction tert-butyl ((R)-1-((R)-5-bromo-7-chlorochroman-4-yl)pyrrolidin-3-yl)carbamate, Rₜ=2.577 min; LCMS R_{T}=0.833 min, m/z=433.0 [M+3+H]+), second eluting fraction tert-butyl ((R)-1-((S)-7-bromo-5-chlorochroman-4-yl)pyrrolidin-3-yl)carbamate, Rₜ=2.877 min; LCMS R_{T}=0.833 min, m/z=433.0 [M+3+H]+), third eluting fraction tert-butyl ((R)-1-((S)-5-bromo-7-chlorochroman-4-yl)pyrrolidin-3-yl)carbamate, Rt=3.227 min; LCMS R_{T}=0.833 min, m/z=433.0 [M+3+H]+) and forth eluting fraction tert-butyl ((R)-1-((R)-7-bromo-5-chlorochroman-4-yl)pyrrolidin-3-yl)carbamate, Rt=3.713 min; LCMS R_{T}=0.833 min, m/z=433.0 [M+2+H]+).

### Step 3: tert-butyl ((R)-1-((S)-5-(2-(((tert-butyldimethylsilyl)oxy)methyl)thieno[3,2-b]pyridin-7-yl)-7-chlorochroman-4-yl)pyrrolidin-3-yl)carbamate

To a solution of tert-butyl N-[(3R)-1-[(4S)-5-bromo-7-chloro-chroman-4-yl]pyrrolidin-3-yl]carbamate (60 mg, 0.14 mmol) in dioxane (1.4 mL) and water (0.2 mL) was added [2-[[tert-butyl(dimethyl)silyl]oxymethyl]thieno[3,2-b]pyridin-7-yl]boronic acid (67 mg, 0.21 mmol), cesium carbonate (135 mg, 0.42 mmol) and palladium dichloride[1,1′-bis (diphenylphosphine) ferrocene] (20 mg, 0.028 mmol). After stirred at 102° C. for 5 h, the reaction mixture was concentrated under reduced pressure. The residue was purified by preparative TLC to afford the title compound. LCMS R_{T}=0.674 min, m/z=630.3 [M+H]⁺.

### Step 4: tert-butyl ((R)-1-((S)-7-chloro-5-(2-(hydroxymethyl)thieno[3,2-b]pyridin-7-yl)chroman-4-yl)pyrrolidin-3-yl)carbamate

Tetrabutylammonium fluoride (0.095 mL, 1 M in THF) in THF (1 mL) was added in a mixture of tert-butyl N-[(1S,3R)-1-[5-[2-[[tert-butyl(dimethyl)silyl]oxymethyl]thieno[3,2-b]pyridin-7-yl]-7-chloro-chroman-4-yl]pyrrolidin-3-yl]carbamate (40 mg, 0.06 mmol) at 25° C. After stirred at 25° C. for 0.5 h, the reaction mixture was concentrated under reduced pressure. The residue was purified by preparative TLC to afford the title compound. LCMS R_{T}=1.010 min, m/z=516.2 [M+H]⁺.

### Step 5

To a solution of diisopropylazodicarboxylate (31 mg, 0.16 mmol) in THF (0.5 mL) was added in a mixture of tert-butyl N-[(1S,3R)-1-[7-chloro-5-[2-(hydroxymethyl)thieno[3,2-b]pyridin-7-yl]chroman-4-yl]pyrrolidin-3-yl]carbamate (27 mg, 0.052 mmol), succinimide (15 mg, 0.16 mmol) and triphenylphosphine (41 mg, 0.16 mmol) at 25° C. After stirred at 45° C. for 1.5 h, the reaction mixture was concentrated under reduced pressure. The residue added Hydrogen chloride (1 mL, 4 M in dioxane) at 25° C. After stirred at 25° C. for 0.1 h, the reaction mixture was concentrated under reduced pressure. The residue was purified by preparative HPLC to afford Example 230a. ¹H NMR (400 MHz, CD₃OD) δ=9.04 (d, J=6.0 Hz, 1H), 8.15 (d, J=6.0 Hz, 1H), 7.88-7.81 (m, 1H), 7.34-7.27 (m, 1H), 7.13 (d, J=2.0 Hz, 1H), 5.15-4.99 (m, 2H), 4.75-4.58 (m, 3H), 4.01 (s, 1H), 3.82-3.52 (m, 3H), 3.46 (d, J=19.8 Hz, 1H), 2.76 (s, 4H), 2.69-2.55 (m, 2H), 2.55-2.43 (m, 1H), 2.22-2.01 (m, 1H). LCMS R_{T}=1.078 min, m/z=497.3 [M+H]⁺.

Example 230b was prepared from tert-butyl ((R)-1-((S)-8-bromo-6-chloro-1,2,3,4-tetrahydronaphthalen-1-yl)pyrrolidin-3-yl)carbamate, following the procedures in the synthesis of Example 230a. The residue was purified by preparative HPLC. ¹H NMR (400 MHz, CD₃OD) δ=9.07-8.89 (m, 1H), 8.05 (d, J=5.2 Hz, 1H), 7.80 (s, 1H), 7.28 (d, J=2.0 Hz, 1H), 7.26-7.08 (m, 1H), 5.13-4.98 (m, 2H), 4.66 (s, 2H), 4.58 (s, 1H), 4.00 (s, 1H), 3.87-3.58 (m, 1H), 3.44 (s, 1H), 3.29-3.21 (m, 1H), 2.93 (s, 1H), 2.83-2.73 (m, 4H), 2.57 (s, 2H), 2.51-2.36 (m, 1H), 2.12 (s, 1H)). LCMS R_{T}=1.068 min, m/z=497.2 [M+H]⁺.

### Example A. Biochemical Assay of USP7 Inhibitor Potency (IC₅₀)

Fluorescence intensity assay of USP7 activity in the presence USP7 inhibitors. Recombinant USP7 protein (catalogue number E-519) and the fluorescence substrate ubiquitin-rhodamine 110 (Rh110) (catalogue number U-555) were purchased from Boston Biochem (840 Memorial Drive, Cambridge, MA 02139). The reaction was carried out in buffer 20 mM HEPES, pH 7.3, 150 mM NaCl, 1 mM TCEP, 125 μg/mL BSA at room temperature in a black 384-well Microplate (Greiner, catalog number 781076).

Test compounds were dissolved in DMSO to make 50 mM stock solution. Echo or POD810 liquid handler was used to dilute the compounds in 10 doses of 2-fold dilution series. In wells of a black 384-well Microplate, 15 μl of 100 pM of USP7 enzyme was added to each well with pre-diluted compounds and incubated at room temperature for 30 minutes, followed by addition of 10 μl of ubiquitin-rhodamine 110 (Rh110) substrate to reach 160 nM final concentration. The reaction mixtures were incubated for additional 90 minutes at room temperature. The fluorescence signal of the digested substrate was measured on an EnVision microplate reader (PerkinElmer,), using excitation at 485 nM and emission at 535 nM. The IC₅₀ values were calculated using XLfit software.

The results are shown in Table 2.

TABLE 2

| | Less than | 10 nM to | 101 nM to | Greater than |
|---|---|---|---|---|
| | 10 nM | 100 nM | 1000 nM | 1000 nM |
| USP7 | 15, 23, 24, 28, 30, | 1, 2, 3, 4, 5, 6, 10, | 7, 8, 11, 12, 21, | 9, 19, 20, 26, 37, |
| biochemical | 42a, 47a, 47b, | 13, 14, 16, 17, 18, | 25, 31, 44b, 44, | 38, 40, 41, 43a, |
| assay IC₅₀ | 51a, 52b, 55, 56, | 22, 27, 29, 32, 33, | 49, 71, 97a, 98, | 111b, 112, 167b, |
| | 57, 59, 60, 61, 62, | 34, 35, 36, 39, | 110, 112b, 112c, | 167, 193a, 227a, |
| | 63, 64, 65, 66, 67, | 42b, 43b, 44a, | 113a, 116, 117a, | 227c, 229b, 230b |
| | 68, 69, 70, 72, 73, | 44c, 45, 46, 48, | 120b, 133b, 143b, | |
| | 74, 75, 76, 77, 79, | 50, 51b, 53, 54, | 156, 160, 161b, | |
| | 81, 82, 83, 84, 87, | 58, 78, 80, 85, 86, | 162b, 164b, 165b, | |
| | 88, 89, 90, 91, 92, | 94, 95b, 96b, 99, | 171a, 173b, 175a, | |
| | 93, 95a, 96a, 97b, | 102, 107b, 108b, | 176a, 178a, 201, | |
| | 100, 101, 103, | 111a, 112a, 113b, | 204, 227b, 227, | |
| | 104, 105, 106, | 113c, 117b, 117c, | 228 | |
| | 107a, 108a, 109, | 118a, 137, 138, | | |
| | 113, 114, 115, | 139a, 143a, 144, | | |
| | 117, 118b, 119, | 147, 152, 155, | | |
| | 120a, 121, 122, | 157, 165a, 167c, | | |
| | 123, 124, 125, | 172, 173a, 179a, | | |
| | 126, 127, 128, | 179b, 180, 181b, | | |
| | 129, 130, 131, | 182, 193b, 194b, | | |
| | 132, 133a, 134, | 196, 199, 200, | | |
| | 135, 136, 139b, | 202, 212b, 213a, | | |
| | 140, 141, 142, | 223, 229a, 230a | | |
| | 145, 146, 148, | | | |
| | 149, 150, 151, | | | |
| | 153, 154, 158, | | | |
| | 159, 161a, 162a, | | | |
| | 163, 164a, 165c, | | | |
| | 165, 166, 167a, | | | |
| | 168, 169, 170, | | | |
| | 171b, 174, 175b, | | | |
| | 176b, 177a, 177b, | | | |
| | 178b, 181a, 183, | | | |
| | 184, 185, 186, | | | |
| | 187, 188, 189, | | | |
| | 190, 191, 192a, | | | |
| | 192b, 194a, 195a, | | | |
| | 195b, 197, 198, | | | |
| | 203, 205, 206, | | | |
| | 207, 208, 209, | | | |
| | 210, 211, 212a, | | | |
| | 213b, 214, 215, | | | |
| | 216, 217, 218, | | | |
| | 219, 220, 221, | | | |
| | 222, 224, 225, | | | |
| | 226 | | | |

### Example B. MM.1S Cell Viability Assay (EC₅₀)

Human peripheral blood B lymphoblast cell line MM.1S was purchased from American Type Culture Collection (ATCC, catalog number ATCC CRL-2974™, 10801 University Boulevard Manassas, VA 20110 USA). The cells were maintained in RIPM 1640 (Gibco, catalog number 22400089) with 10% fetal bovine serum (Gibco, catalog number 10099-141C) and 100 of Penicillin and Streptomycin (Hyclone, catalogue number SV30010) in a humidified cell culture incubator at 37° C. with 5% CO₂.

Results of the MM.1S viability assay (IC₅₀) are presented in Table 3.

TABLE 3

| | Less than | 100 nM to | 251 nM to | Greater than |
|---|---|---|---|---|
| | 100 nM | 250 nM | 1000 nM | 1000 nM |
| MM.1S cell | 55, 65, 72, 83, 84, | 51a, 61, 62, 63, | 1, 2, 15, 23, 24, | 3, 4, 5, 10, 11, 13, |
| viability assay | 87, 88, 89, 92, | 64, 66, 67, 75, 79, | 36, 39, 42a, 44c, | 14, 16, 17, 18, 22, |
| IC₅₀ | 95a, 103, 117, | 81, 82, 91, 96a, | 46, 47a, 47b, 56, | 27, 28, 29, 30, 32, |
| | 120a, 121, 123, | 97b, 100, 101, | 57, 59, 60, 68, 69, | 34, 35, 42b, 43b, |
| | 125, 126, 127, | 104, 106, 107a, | 70, 74, 76, 77, 86, | 45, 51b, 52b, 53, |
| | 128, 129, 132, | 109, 114, 115, | 90, 93, 95b, 105, | 54, 58, 73, 80, |
| | 133a, 136, 139b, | 118b, 119, 122, | 107b, 108a, 113, | 96b, 108b, 110, |
| | 140, 141, 145, | 130, 131, 134, | 117c, 124, 142, | 111a, 111b, 112a, |
| | 146, 148, 149, | 135, 151, 158, | 147, 155, 159, | 112b, 112c, 112, |
| | 150, 153, 154, | 166, 171b, 176b, | 161a, 165c, 173a, | 113b, 116, 117b, |
| | 162a, 163, 164a, | 185, 197, 213b, | 174, 175b, 177a, | 144, 160, 165, |
| | 168, 169, 177b, | 217, 224 | 178b, 184, 187, | 167a, 170, 178a, |
| | 181a, 183, 186, | | 192b, 194b, 195b, | 179a, 179b, 180, |
| | 188, 189, 190, | | 196, 206, 208, | 182, 193a, 193b, |
| | 191, 192a, 194a, | | 214, 220, 223, | 199, 200, 201, |
| | 195a, 198, 207, | | 225, 226 | 202, 203, 204, |
| | 209, 210, 211, | | | 205, 212a, 212b, |
| | 216, 219, 221, | | | 213a, 215, 218, |
| | 222 | | | 227a, 227b, 227c, |
| | | | | 227, 228, 229a, |
| | | | | 229b, 230a |

### Example C. Induction of TP53 Expression by USP7 inhibitors

Total 1.0×10⁶ MM.1S cells were seeded into each well of a 6-well plate (Corning, catalogue number 3506) 16-24 hours prior to compound treatment. The cells were treated with compounds diluted in DMSO in 8 doses by 4-fold steps with top concentration at 20 DM for 4 hours under normal cell culture condition, before the cell lysates were prepared using MSD Tris lysis buffer (catalogue number R60TX). Compound MI-773 (synthesized by WuXi chemists) was used as reference for quality control. The total protein concentrations in the cell lysates were determined using Thermo Scientific™ Pierce™ BCA Protein Assay Kit (Thermo Scientific™, catalogue number 23227). Total p53 Whole Cell Lysate Kit (catalogue number K150DBD) from Meso Scale Discovery (1601 Research Boulevard, Rockville, Maryland 20850-3173) was used to measure the relative amount of TP53 protein in each cell lysate following the manufacturer's instructions. The EC₅₀ values were calculated using GraphPad Prism (2365 Northside Dr. Suite 560, San Diego, CA 92108).

Results of the MM.1S p53 induction assay (EC₅₀) are presented in Table 4.

TABLE 4

| | Less than | 100 nM to | 251 nM to | Greater than |
|---|---|---|---|---|
| | 100 nM | 250 nM | 1000 nM | 1000 nM |
| MM.1S p53 MSD | 47a, 51a, 55, 57, | 15, 24, 32, 36, 46, | 2, 4, 10, 16, 22, | 1, 3, 14, 27, 28, |
| assay EC₅₀ | 59, 72, 75, 76, 79, | 60, 61, 62, 63, 67, | 23, 30, 43b, 47b, | 56, 170 |
| | 82, 83, 84, 87, 88, | 73, 74, 89, 90, | 52b, 58, 66, 70, | |
| | 91, 92, 95a, 97b, | 96a, 100, 106, | 77, 81, 93, 108a, | |
| | 101, 103, 104, | 109, 165c, 187, | 113, 114, 203 | |
| | 115, 117, 118b, | 196, 214, 215 | | |
| | 119, 120a, 121, | | | |
| | 122, 123, 124, | | | |
| | 126, 127, 128, | | | |
| | 129, 131, 132, | | | |
| | 133a, 134, 135, | | | |
| | 136, 139b, 140, | | | |
| | 141, 145, 146, | | | |
| | 148, 149, 150, | | | |
| | 151, 153, 154, | | | |
| | 158, 159, 161a, | | | |
| | 162a, 163, 164a, | | | |
| | 166, 168, 169, | | | |
| | 171b, 174, 176b, | | | |
| | 177a, 177b, 178b, | | | |
| | 181a, 183, 184, | | | |
| | 185, 186, 188, | | | |
| | 189, 190, 191, | | | |
| | 192a, 194a, 195a, | | | |
| | 197, 198, 206, | | | |
| | 207, 209, 210, | | | |
| | 211, 212a, 213b, | | | |
| | 216, 217, 218, | | | |
| | 219, 221, 222, | | | |
| | 224, 226 | | | |

The examples and embodiments described herein are for illustrative purposes only and in some embodiments, various modifications or changes are to be included within the purview of disclosure and scope of the appended claims.

What is claimed is:

1. A compound of Formula (I), or a pharmaceutically acceptable salt, solvate, N-oxide, or stereoisomer thereof: 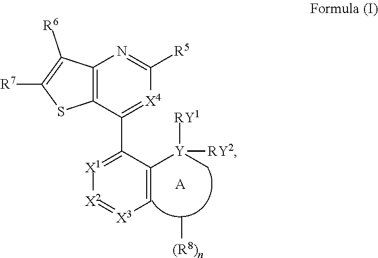
wherein:
Ring A is 5-, 6-, or 7-membered cycloalkyl or heterocycloalkyl;
Y is N; RY¹ is absent; and RY² is -L-R^{A};
or Y is C; RY¹ is hydrogen, deuterium, halogen, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; and RY² is -L-R^{A};
or Y is C and RY¹ and RY² are taken together with Y to form Ring B optionally substituted with one or more R^{B};
L is a bond or C₁-C₆alkylene optionally substituted with one or more deuterium, halogen, —CN, —OR^{b}, —NR^{c}R^{d}, —C(═O)R^{a}, —C(═O)OR^{b}, or —C(═O)NR^{c}R^{d};
R^{A} is —NR^{c}R^{d}, cycloalkyl, heterocycloalkyl, aryl, or heteroaryl; wherein the cycloalkyl, heterocycloalkyl, aryl, and heteroaryl is optionally substituted with one or more R^{Aa};
each R^{Aa} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, alkenyl, alkynyl, cycloalkyl, heterocycloalkyl, aryl, and heteroaryl is optionally substituted with one or more R^{Aaa};
or two R^{Aa} are taken together to form an aryl, heteroaryl, cycloalkyl, or heterocycloalkyl; wherein the aryl, heteroaryl, cycloalkyl, and heterocycloalkyl is optionally substituted with one or more R^{Aaa};
or two R^{Aa} on the same carbon are taken together to form an oxo;
Ring B is cycloalkyl or heterocycloalkyl;
each R^{B} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{B} on the same carbon are taken together to form an oxo;
X¹ is N or CR¹;
R¹ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{1a};
X² is N or CR²;
R² is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
X³ is N or CR³;
R³ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{3a};
X⁴ is N or CR⁴;
R⁴ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{4a};
R⁵ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{5a};
R⁶ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{6a};
R⁷ is hydrogen, deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, alkenyl, alkynyl, cycloalkyl, heterocycloalkyl, aryl, and heteroaryl is optionally substituted with one or more R^{7a};
each R^{7a} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, alkenyl, alkynyl, cycloalkyl, heterocycloalkyl, aryl, and heteroaryl is optionally substituted with one or more R^{7aa};
or two R^{7a} are taken together to form an aryl, heteroaryl, cycloalkyl, or heterocycloalkyl; wherein the aryl, heteroaryl, cycloalkyl, and heterocycloalkyl is optionally substituted with one or more R^{7aa};
or two R^{7a} on the same carbon are taken together to form an oxo;
each R⁸ is independently hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{5a};
or two R⁸ are taken together to form a cycloalkyl or a heterocycloalkyl; each optionally substituted with one or more R^{5a};
or two R⁸ on the same carbon are taken together to form an oxo;
n is 1-4;
each R^{1a}, R^{2a}, R^{3a}, R^{4a}, R^{5a}, R^{6a}, and R^{8a} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{1a}, or two R^{2a}, or two R^{3a}, or two R^{4a}, or two R^{5a}, or two R^{6a}, or two R^{8a} are taken together to form an a cycloalkyl or a heterocycloalkyl;
or two R^{1a}, or two R^{2a}, or two R^{3a}, or two R^{4a}, or two R^{5a}, or two R^{6a}, or two R^{8a} on the same carbon are taken together to form an oxo;
each R^{7aa} and R^{Aaa} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{7aa} or two R^{Aa} on the same carbon are taken together to form an oxo;
each R^{a} is independently C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
each R^{b} is independently hydrogen, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; and
each R^{c} and R^{d} is independently hydrogen, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl.

2. The compound of claim 1, or a pharmaceutically acceptable salt, solvate, N-oxide, or stereoisomer thereof wherein:
Y is N; RY¹ is absent; and RY² is -L-R^{A}.

3. The compound of claim 1, or a pharmaceutically acceptable salt, solvate, N-oxide, or stereoisomer thereof wherein:
Y is C; RY¹ is hydrogen, deuterium, halogen, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl; and RY² is -L-R^{A}.

4. The compound of claim 1, or a pharmaceutically acceptable salt, solvate, N-oxide, or stereoisomer thereof wherein:
Y is C; RY¹ is hydrogen; and RY² is -L-R^{A}.

5. The compound of any one of claims 1-4, or a pharmaceutically acceptable salt, solvate, N-oxide, or stereoisomer thereof wherein:
L is a bond.

6. The compound of any one of claims 1-4, or a pharmaceutically acceptable salt, solvate, N-oxide, or stereoisomer thereof wherein:
L is C₁-C₆alkylene optionally substituted with one or more deuterium, halogen, —CN, —OR^{b}, —NR^{c}R^{d}, —C(═O)R^{a}, —C(═O)OR, or —C(═O)NR^{c}R^{d}.

7. The compound of any one of claims 1-4, or a pharmaceutically acceptable salt, solvate, N-oxide, or stereoisomer thereof wherein:
L is CH₂.

8. The compound of claim 1, or a pharmaceutically acceptable salt, solvate, N-oxide, or stereoisomer thereof wherein:
Y is C and RY¹ and RY² are taken together with Y to form Ring B optionally substituted with one or more R^{B}.

9. The compound of claim 1 or 8, or a pharmaceutically acceptable salt, solvate, N-oxide, or stereoisomer thereof wherein:
Ring B is cycloalkyl.

10. The compound of claim 1 or 8, or a pharmaceutically acceptable salt, solvate, N-oxide, or stereoisomer thereof wherein:
Ring B is heterocycloalkyl.

11. The compound of any one of claims 1 or 8-10, or a pharmaceutically acceptable salt, solvate, N-oxide, or stereoisomer thereof wherein:
each R^{B} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, or C₁-C₆aminoalkyl.

12. The compound of any one of claims 1-11, or a pharmaceutically acceptable salt, solvate, N-oxide, or stereoisomer thereof wherein:
Ring A is 6-membered heterocycloalkyl.

13. The compound of any one of claims 1-12, or a pharmaceutically acceptable salt, solvate, N-oxide, or stereoisomer thereof wherein:
Ring A is piperidine, piperazine, morpholine, or tetrahydropyran.

14. The compound of any one of claims 1, 2, 12, or 13, or a pharmaceutically acceptable salt, solvate, N-oxide, or stereoisomer thereof wherein the compound of Formula (I) is of Formula (Ia):

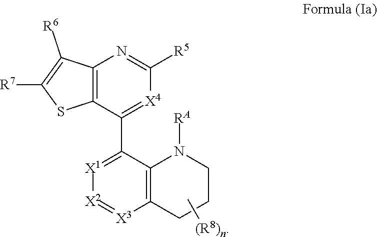

15. The compound of any one of claims 1-14, or a pharmaceutically acceptable salt, solvate, N-oxide, or stereoisomer thereof wherein:
X¹ is N.

16. The compound of any one of claims 1-14, or a pharmaceutically acceptable salt, solvate, N-oxide, or stereoisomer thereof wherein:
X¹ is CR¹.

17. The compound of any one of claims 1-16, or a pharmaceutically acceptable salt, solvate, N-oxide, or stereoisomer thereof wherein:
X² is N.

18. The compound of any one of claims 1-16, or a pharmaceutically acceptable salt, solvate, N-oxide, or stereoisomer thereof wherein:
X² is CR².

19. The compound of any one of claims 1-18, or a pharmaceutically acceptable salt, solvate, N-oxide, or stereoisomer thereof wherein:
X³ is N.

20. The compound of any one of claims 1-18, or a pharmaceutically acceptable salt, solvate, N-oxide, or stereoisomer thereof wherein:
X³ is CR³.

21. The compound of any one of claims 1-20, or a pharmaceutically acceptable salt, solvate, N-oxide, or stereoisomer thereof wherein:
X⁴ is N.

22. The compound of any one of claims 1-20, or a pharmaceutically acceptable salt, solvate, N-oxide, or stereoisomer thereof wherein:
X⁴ is CR⁴.

23. The compound of any preceeding claims, or a pharmaceutically acceptable salt, solvate, N-oxide, or stereoisomer thereof wherein the compound of Formula (I) or (Ia) is of Formula (Ib):

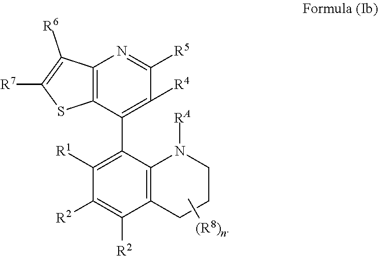

24. The compound of any preceeding claims, or a pharmaceutically acceptable salt, solvate, N-oxide, or stereoisomer thereof wherein:
R¹ is hydrogen, halogen, —CN, C₁-C₆alkyl, or C₁-C₆haloalkyl.

25. The compound of any preceeding claims, or a pharmaceutically acceptable salt, solvate, N-oxide, or stereoisomer thereof wherein:
R¹ is hydrogen.

26. The compound of any preceeding claims, or a pharmaceutically acceptable salt, solvate, N-oxide, or stereoisomer thereof wherein:
R² is hydrogen, halogen, —CN, C₁-C₆alkyl, or C₁-C₆haloalkyl.

27. The compound of any preceeding claims, or a pharmaceutically acceptable salt, solvate, N-oxide, or stereoisomer thereof wherein:
R² is halogen.

28. The compound of any preceeding claims, or a pharmaceutically acceptable salt, solvate, N-oxide, or stereoisomer thereof wherein:
R³ is hydrogen, halogen, —CN, C₁-C₆alkyl, or C₁-C₆haloalkyl.

29. The compound of any preceeding claims, or a pharmaceutically acceptable salt, solvate, N-oxide, or stereoisomer thereof wherein:
R³ is hydrogen.

30. The compound of any preceeding claims, or a pharmaceutically acceptable salt, solvate, N-oxide, or stereoisomer thereof wherein:
R⁴ is hydrogen, halogen, —CN, C₁-C₆alkyl, or C₁-C₆haloalkyl.

31. The compound of any preceeding claims, or a pharmaceutically acceptable salt, solvate, N-oxide, or stereoisomer thereof wherein:
R⁴ is hydrogen.

32. The compound of any one of claims 1-31, or a pharmaceutically acceptable salt, solvate, N-oxide, or stereoisomer thereof wherein:
R⁵ is hydrogen, halogen, —CN, C₁-C₆alkyl, or C₁-C₆haloalkyl.

33. The compound of any one of claims 1-32, or a pharmaceutically acceptable salt, solvate, N-oxide, or stereoisomer thereof wherein:
R⁵ is hydrogen.

34. The compound of any one of claims 1-33, or a pharmaceutically acceptable salt, solvate, N-oxide, or stereoisomer thereof wherein:
R⁶ is hydrogen, halogen, —CN, C₁-C₆alkyl, or C₁-C₆haloalkyl.

35. The compound of any one of claims 1-34, or a pharmaceutically acceptable salt, solvate, N-oxide, or stereoisomer thereof wherein:
R⁶ is hydrogen.

36. The compound of any one of claims 1-35, or a pharmaceutically acceptable salt, solvate, N-oxide, or stereoisomer thereof wherein:
R⁷ is —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, cycloalkyl, heterocycloalkyl, aryl, and heteroaryl is optionally substituted with one or more R^{a}.

37. The compound of any one of claims 1-36, or a pharmaceutically acceptable salt, solvate, N-oxide, or stereoisomer thereof wherein:
R⁷ is cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, cycloalkyl, heterocycloalkyl, aryl, and heteroaryl is optionally substituted with one or more R^{7a}.

38. The compound of any one of claims 1-37, or a pharmaceutically acceptable salt, solvate, N-oxide, or stereoisomer thereof wherein:
R⁷ is (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, cycloalkyl, heterocycloalkyl, aryl, and heteroaryl is optionally substituted with one or more R^{7a}.

39. The compound of any one of claims 1-38, or a pharmaceutically acceptable salt, solvate, N-oxide, or stereoisomer thereof wherein:
R⁷ is (C₁-C₆alkyl)heterocycloalkyl or (C₁-C₆alkyl)heteroaryl; wherein the alkyl, heterocycloalkyl, and heteroaryl is optionally substituted with one or more R^{7a}.

40. The compound of any one of claims 1-39, or a pharmaceutically acceptable salt, solvate, N-oxide, or stereoisomer thereof wherein:
R⁷ is (C₁-C₆alkyl)heterocycloalkyl optionally substituted with one or more R^{7a}.

41. The compound of any one of claims 1-40, or a pharmaceutically acceptable salt, solvate, N-oxide, or stereoisomer thereof wherein:
each R^{7a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, cycloalkyl, heterocycloalkyl, aryl, and heteroaryl is optionally substituted with one or more R^{7aa}; or two R^{7a} are taken together to form an aryl, heteroaryl, cycloalkyl, or heterocycloalkyl; wherein the aryl, heteroaryl, cycloalkyl, and heterocycloalkyl is optionally substituted with one or more R^{7aa}; or two R^{7a} on the same carbon are taken together to form an oxo.

42. The compound of any one of claims 1-41, or a pharmaceutically acceptable salt, solvate, N-oxide, or stereoisomer thereof wherein:
each R^{7a} is independently halogen, —OR^{b}, —NR^{c}R^{d}, C₁-C₆alkyl, C₁-C₆haloalkyl, C₁-C₆heteroalkyl, C₁-C₆hydroxyalkyl, cycloalkyl, heterocycloalkyl, aryl, heteroaryl, (C₁-C₆alkyl)cycloalkyl, (C₁-C₆alkyl)heterocycloalkyl, (C₁-C₆alkyl)aryl, or (C₁-C₆alkyl)heteroaryl; wherein the alkyl, cycloalkyl, heterocycloalkyl, aryl, and heteroaryl is optionally substituted with one or more R^{7aa}; or two R^{7a} are taken together to form an aryl or cycloalkyl; wherein the aryl and cycloalkyl is optionally substituted with one or more R^{7aa}; or two R^{7a} on the same carbon are taken together to form an oxo.

43. The compound of any one of claims 1-42, or a pharmaceutically acceptable salt, solvate, N-oxide, or stereoisomer thereof wherein:
each R^{7a} is independently halogen, —OR^{b}, —NR^{c}R^{d}, C₁-C₆alkyl, C₁-C₆haloalkyl, C₁-C₆heteroalkyl, C₁-C₆hydroxyalkyl, cycloalkyl, heterocycloalkyl, heteroaryl, (C₁-C₆alkyl)cycloalkyl, or (C₁-C₆alkyl)heteroaryl; wherein the alkyl, cycloalkyl, heterocycloalkyl, and heteroaryl is optionally substituted with one or more R^{7aa}; or two R^{7a} on the same carbon are taken together to form an oxo.

44. The compound of any one of claims 1-43, or a pharmaceutically acceptable salt, solvate, N-oxide, or stereoisomer thereof wherein:
two R^{7a} on the same carbon are taken together to form an oxo.

45. The compound of any one of claims 1-44, or a pharmaceutically acceptable salt, solvate, N-oxide, or stereoisomer thereof wherein:
each R^{7aa} is independently halogen, —CN, —OR^{b}, —NR^{c}R^{d}, —C(═O)R^{a}, —C(═O)OR^{b}, —C(═O)NR^{c}R^{d}, C₁-C₆alkyl, or C₁-C₆haloalkyl.

46. The compound of any one of claims 1-45, or a pharmaceutically acceptable salt, solvate, N-oxide, or stereoisomer thereof wherein:
each R^{7aa} is independently halogen, —CN, —OR^{b}, C₁-C₆alkyl, or C₁-C₆haloalkyl.

47. The compound of any one of claims 1-46, or a pharmaceutically acceptable salt, solvate, N-oxide, or stereoisomer thereof wherein:
each R⁸ is independently hydrogen, deuterium, halogen, C₁-C₆alkyl, or C₁-C₆haloalkyl; or two R are taken together to form a cycloalkyl or a heterocycloalkyl; or two R⁸ on the same carbon are taken together to form an oxo.

48. The compound of any one of claims 1-47, or a pharmaceutically acceptable salt, solvate, N-oxide, or stereoisomer thereof wherein:
two R⁸ are taken together to form a cycloalkyl.

49. The compound of any one of claims 1-48, or a pharmaceutically acceptable salt, solvate, N-oxide, or stereoisomer thereof wherein:
n is 1-3.

50. The compound of any one of claims 1-49, or a pharmaceutically acceptable salt, solvate, N-oxide, or stereoisomer thereof wherein:
n is 1 or 2.

51. The compound of any one of claims 1-50 or a pharmaceutically acceptable salt, solvate, N-oxide, or stereoisomer thereof wherein:
R^{A} is NR^{c}R^{d}.

52. The compound of any one of claims 1-50, or a pharmaceutically acceptable salt, solvate, N-oxide, or stereoisomer thereof wherein:
R^{A} is heterocycloalkyl optionally substituted with one or more R^{Aa}.

53. The compound of any one of claims 1-50 or 52, or a pharmaceutically acceptable salt, solvate, N-oxide, or stereoisomer thereof wherein:
each R^{Aa} is independently halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆heteroalkyl, C₁-C₆hydroxyalkyl, C₁-C₆aminoalkyl, cycloalkyl, or heterocycloalkyl; wherein the alkyl, cycloalkyl, and heterocycloalkyl is optionally substituted with one or more R^{Aaa}.

54. The compound of any one of claims 1-50 or 52 or 53, or a pharmaceutically acceptable salt, solvate, N-oxide, or stereoisomer thereof wherein:
each R^{Aa} is independently halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, cycloalkyl, or heterocycloalkyl; wherein the alkyl, cycloalkyl, and heterocycloalkyl is optionally substituted with one or more R^{Aaa}.

55. The compound of any one of claims 1-50 or 52-54, or a pharmaceutically acceptable salt, solvate, N-oxide, or stereoisomer thereof wherein:
each R^{Aaa} is independently halogen, —CN, —OR^{b}, C₁-C₆alkyl, or C₁-C₆haloalkyl.

56. The compound of claim 1, or a pharmaceutically acceptable salt, solvate, N-oxide, or stereoisomer thereof selected from a compound in table 1.

57. A pharmaceutical composition comprising a compound of any one of claims 1-56, or a pharmaceutically acceptable salt, solvate, N-oxide, or stereoisomer thereof, and a pharmaceutically acceptable excipient.

58. The pharmaceutical composition of claim 57, for use in treating cancer modulated by ubiquitin specific protease 7 (USP7).

59. The pharmaceutical composition of claim 58, for use in treating solid tumor or blood cancer.

60. The pharmaceutical composition of claim 59, wherein the solid tumor or blood cancer is ovarian cancer, breast cancer, lung cancer, pancreatic cancer, kidney cancer, melanoma, liver cancer, colon cancer, sarcoma, brain cancer, prostate cancer, leukemia, lymphoma, or multiple myeloma.

61. A method of treating cancer comprising administering to a subject in need thereof a therapeutically effective amount of a compound of any one of claims 1-56, or a pharmaceutically acceptable salt, solvate, N-oxide, or stereoisomer thereof.

62. The method of claim 61, wherein the cancer is a solid tumor.

63. The method of claim 62, wherein the solid tumor is ovarian cancer, breast cancer, lung cancer, pancreatic cancer, kidney cancer, melanoma, liver cancer, colon cancer, sarcoma, brain cancer, or prostate cancer.

64. The method of claim 61, wherein the cancer is a blood cancer.

65. The method of claim 64, wherein the blood cancer is leukemia, lymphoma, or multiple myeloma.

66. The method of claim 65, wherein the leukemia is acute myeloid leukemia (AML).

67. The method of claim 64, wherein the blood cancer is a myeloproliferative neoplasm (MPN).

68. The method of claim 67, wherein the MPN is chronic myeloid leukemia (CML), chronic neutrophilic leukemia (CNL), polycythemia vera (PV), primary myelofibrosis (PMF), essential thrombocythemia (ET), or chronic eosinophilic leukemia.

69. The method of any one of claims 61-68, further comprising administering to the subject in need thereof an additional therapeutic agent.

70. The method of claim 69, wherein the additional therapeutic agent is a histone deacetylase (HDAC) inhibitor, a proteasome inhibitor, a BET inhibitor, a B-cell lymphoma 2 (Bcl-2) inhibitor, or any combination thereof.

71. The method of claim 69, wherein the additional therapeutic agent is an immunotherapy agent.

72. The method of claim 71, wherein the immunotherapy agent is an immune checkpoint inhibitor.

73. The method of claim 72, wherein the immune checkpoint inhibitor is a PD-1 inhibitor, a PD-L1 inhibitor, a PD-L2 inhibitor, a CTLA-4 inhibitor, a OX40 agonist, or a 4-1BB agonist.
